(19) United States
(12) Patent Application Publication
(10) Pub. No.: US 2024/0150365 A1
(43) Pub. Date: May 9, 2024

(54) PYRIDINYLSULFONAMIDE COMPOUNDS AND THEIR USE IN THERAPY

(71) Applicant: Hotspot Therapeutics, Inc., Boston, MA (US)

(72) Inventors: Yingzhi BI, Plainsboro, NJ (US); Kenneth G. CARSON, Princeton, NJ (US); Geraldine Cirillo HARRIMAN, Charlestown, RI (US); Sundarapandian THANGAPANDIAN, Foxboro, MA (US); Christian Josef KUPER, Hertford (GB); Sebastien Louis DEGORCE, Saffron Walden (GB); Arwel LEWIS, Royston (GB); Maria Angel PALOMERO-VAZQUEZ, Whittlesford (GB); Robert Stuart Laurie CHAPMAN, Saffron Walden (GB)

(21) Appl. No.: 18/237,989

(22) Filed: Aug. 25, 2023

Related U.S. Application Data
(60) Provisional application No. 63/401,491, filed on Aug. 26, 2022.

(51) Int. Cl. C07D 487/06; C07D 401/14; C07D 403/14; C07D 413/14; C07D 417/14; C07D 471/04; C07D 491/052; C07D 498/04; C07D 519/00

(52) U.S. Cl. CPC C07D 487/06; C07D 401/14; C07D 403/14; C07D 413/14; C07D 417/14; C07D 471/04; C07D 491/052; C07D 498/04; C07D 519/00

(57) ABSTRACT

The invention provides pyridinylsulfonamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder.

### CROSS REFERENCE TO RELATED APPLICATIONS

This application claims the benefit of and priority to U.S. Provisional Patent Application Ser. No. 63/401,491, filed Aug. 26, 2022; the contents of which are hereby incorporated by reference in their entirety.

### FIELD OF THE INVENTION

The invention provides pyridinylsulfonamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. Current treatment options for these cancers are not effective for all patients and/or can have substantial adverse side effects. Moreover, new therapies that achieve an anti-cancer effect through a different mechanism present an opportunity to treat cancers more effectively and/or to treat cancers that have become resistant to currently available medicines.

Inflammatory disorders impact a substantial number of patients and often involve situations where the patient's biological response to a stimulus results in the immune system attacking the body's own cells or tissues. This can lead to abnormal inflammation and result in chronic pain, redness, swelling, stiffness, and/or damage to normal tissues. Current treatment options for these inflammatory disorders are not effective for all patients and/or can have substantial adverse side effects.

Human mucosa-associated lymphoid tissue protein 1 (MALT1) is a key regulator of immune responses and is an immune modulatory target for the treatment of autoimmune and inflammatory diseases. In addition, research indicates that MALT1 inhibition impairs immune suppressive function of regulatory T cells in a tumor microenvironment, implicating MALT1 inhibitors for boosting anti-tumor immunity in the treatment of solid cancers. See, for example, Isabel Hamp et al. in Expert Opinion on Therapeutic Patents (2021) vol. 12, pages 1079-1096.

The present invention addresses the foregoing needs and provides other related advantages.

### SUMMARY

The invention provides pyridinylsulfonamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder. In particular, one aspect of the invention provides a collection of pyridinylsulfonamide compounds, such as a compound represented by Formula I:

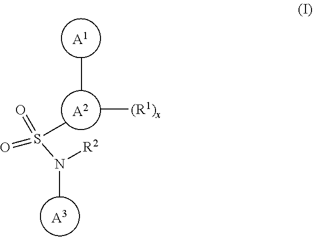

or a pharmaceutically acceptable salt thereof, where the variables are as defined in the detailed description. Further description of additional collections of pyridinylsulfonamide compounds are described in the detailed description. The compounds may be part of a pharmaceutical composition comprising a pharmaceutically acceptable carrier.

Another aspect of the invention provides a collection of pyridinylsulfonamide compounds, such as a compound represented by Formula II:

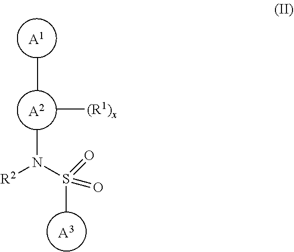

or a pharmaceutically acceptable salt thereof, where the variables are as defined in the detailed description. Further description of additional collections of pyridinylsulfonamide compounds are described in the detailed description. The compounds may be part of a pharmaceutical composition comprising a pharmaceutically acceptable carrier.

Another aspect of the invention provides a method of treating a disease or condition mediated by MALT1 in a subject. The method comprises administering a therapeutically effective amount of a compound described herein, such as a compound of Formula I, I-1, I-2, I-3, I-4, or II, or other compounds in section I, to a subject in need thereof to treat the disease or condition, as further described in the detailed description.

Another aspect of the invention provides a method of inhibiting the activity of MALT1. The method comprises contacting a MALT1 with an effective amount of a compound described herein, such as a compound of Formula I, I-1, I-2, I-3, I-4, II, or other compounds in section I, to inhibit the activity of said MALT1, as further described in the detailed description.

### DETAILED DESCRIPTION

The invention provides pyridinylsulfonamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder. The practice of the present invention employs, unless otherwise indicated, conventional techniques of organic chemistry, pharmacology, molecular biology (including recombinant techniques), cell biology, biochemistry, and immunology. Such techniques are explained in the literature, such as in Comprehensive Organic Synthesis (B. M. Trost & I. Fleming, eds., 1991-1992); Handbook of Experimental Immunology (D. M. Weir & C. C. Blackwell, eds.); Current Protocols in Molecular Biology (F. M. Ausubel et al., eds., 1987, and periodic updates); and Current Protocols in Immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In certain embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In certain embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In certain embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

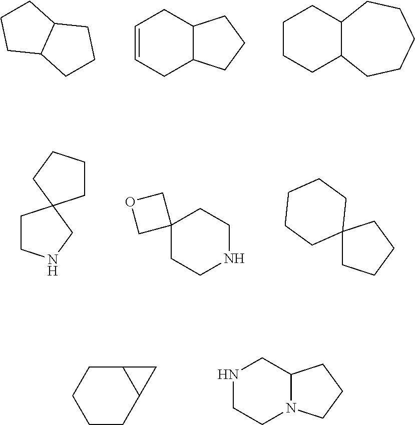

Exemplary bridged bicyclics include:

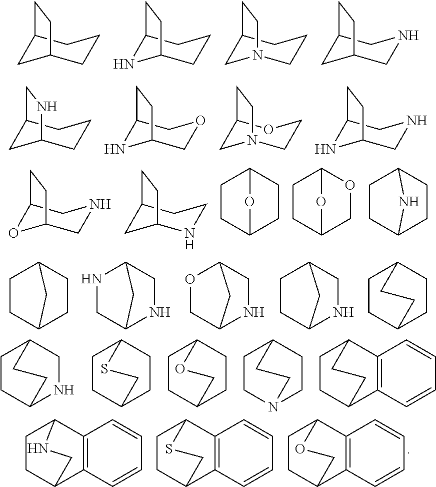

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including any oxidized form of nitrogen, sulfur, phosphorus, or silicon; the quaternized form of any basic nitrogen; or a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁-6) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” or “halo” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but is not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

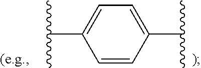 [SMILES: *c1ccc(*)cc1]

“phenylene” is a trivalent phenyl group when it has three groups attached to it

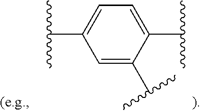 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it. The term “pyridinylene” refers to a multivalent pyridine radical having the appropriate number of open valences to account for groups attached to it. For example, “pyridinylene” is a bivalent pyridine radical when it has two groups attached to it

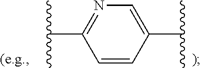 [SMILES: *c1ccc(*)nc1]

“pyridinylene” is a trivalent pyridine radical when it has three groups attached to it

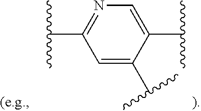 [SMILES: *c1cc(*)c(*)cn1]

The term “pyridazinylene” refers to a multivalent pyridazine radical having the appropriate number of open valences to account for groups attached to it. For example, “pyridazinylene” is a bivalent pyridazine radical when it has two groups attached to it

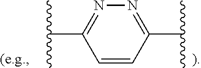 [SMILES: *c1ccc(*)nn1]

The term “pyrimidinylene” refers to a multivalent pyrimidine radical having the appropriate number of open valences to account for groups attached to it. For example, “pyrimidinylene” is a bivalent pyrimidine radical when it has two groups attached to it

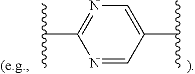 [SMILES: *c1cnc(*)nc1]

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by an oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}2; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R, —S(O)₂NR₂, —C(S)NR^{†}₂, —C(NH)NR^{†}₂, or —N(RT)S(O)₂R; wherein each R is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences 1977, 66(1), 1-19; P. Gould, International J. of Pharmaceutics 1986, 33, 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

Unless specified otherwise, the term “about” refers to within ±10% of the stated value. The invention encompasses embodiments where the value is within ±9%, ±8%, ±7%, ±6%, ±5%, ±4%, ±3%, ±2%, or ±1% of the stated value.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The terms “alkenyl” and “alkynyl” are art-recognized and refer to unsaturated aliphatic groups analogous in length and possible substitution to the alkyls described above, but that contain at least one double or triple bond, respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like. The term “hydroxyalkoxyl” refers to an alkoxyl group that is substituted with at least one hydroxyl. Exemplary hydroxyalkoxyl groups include —OCH₂CH₂OH, —OCH₂C(H)(OH)CH₂CH₂OH, and the like. The term “alkoxylene” refers to a bivalent alkoxyl group.

The term “oxo” is art-recognized and refers to a “═O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and, most preferably, include humans.

As used herein, the term “compound” refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate-buffered saline solution, water, emulsions (e.g., such as oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. PYRIDINYLSULFONAMIDE COMPOUNDS

The invention provides pyridinylsulfonamide compounds. The compounds may be used in the pharmaceutical compositions and therapeutic methods described herein. Exemplary compounds are described in the following sections, along with exemplary procedures for making the compounds.

### Part A:

One aspect of the invention provides a compound represented by Formula I:

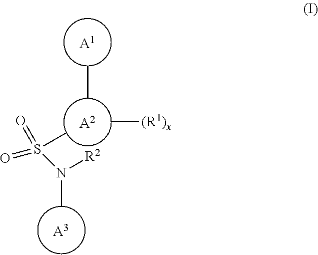

or a pharmaceutically acceptable salt thereof, wherein:
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, C₂₋₄ aminoalkyl, —(C₁₋₄ alkylene)-(C₁₋₆ alkoxyl), or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl substituted with 0 or 1 occurrences of C₁₋₆ alkoxyl, C₃₋₇ halocycloalkyl, C₃₋₇ hydroxycycloalkyl, —O—C₃₋₇ cycloalkyl, nitro, —C(O)R⁷, —N(R⁸)(R⁹), oxo, C₁₋₄ hydroxyalkyl, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, C₁₋₄ alkyl, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, 3, or 4 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered saturated heterocyclyl containing 1, 2 or 3 heteroatoms independently selected from oxygen, nitrogen, and sulfur, or —N(R²)-(5-6 membered saturated carbocylic ring substituted with n occurrences of R⁶), wherein the heteroaryl and heterocyclyl are substituted with n occurrences of R₆;
A² is a pyridinylene, pyridazinylene, pyrimidinylene, or phenylene;
A³ is

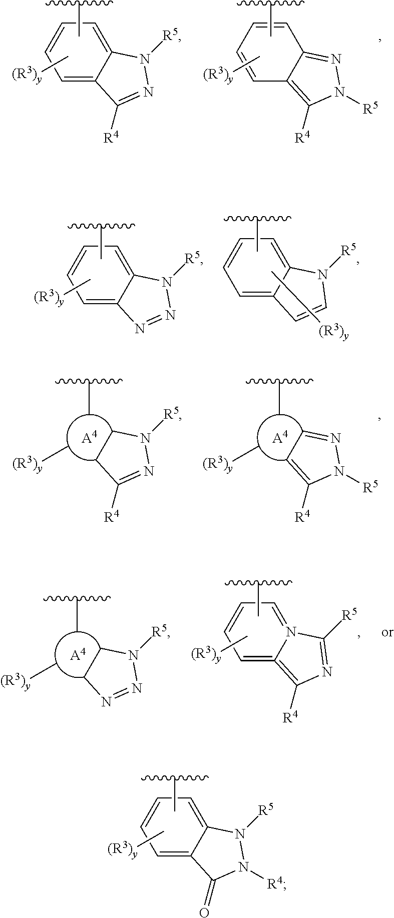

A⁴ is a 6-membered aromatic ring containing 1 nitrogen atom;
y is 0, 1, or 2;
m and x are independently 0, 1, or 2; and
n is 0, 1, 2, or 3;
provided that if A² is phenylene, then y is 1 or 2 and at least one occurrence of R³ is C₁₋₆ alkoxyl, C₁₋₆ deuteroalkoxyl, or C₃₋₇ cycloalkyl.

The definitions of variables in Formula I above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I.

As defined generally above, R¹ represents independently for each occurrence halo, C₁₋₄ alkyl, C₁₋₄ haloalkyl, or cyano. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is C₁₋₄ haloalkyl. In certain embodiments, R¹ is C₁ haloalkyl. In certain embodiments, R¹ is C₂haloalkyl. In certain embodiments, R¹ is C₃ haloalkyl. In certain embodiments, R¹ is C₄ haloalkyl. In certain embodiments, R¹ is cyano. In certain embodiments, R¹ is selected from the groups depicted in the compounds in Table 1 below.

As defined generally above, R² is hydrogen, C₁₋₄ alkyl, C₂₋₄ hydroxyalkyl, or —(C₁-6 alkylene)-N(R⁸)(R⁹). In certain embodiments, R² is hydrogen. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is C₂₋₄ hydroxyalkyl. In certain embodiments, R² is C₂ hydroxyalkyl. In certain embodiments, R² is C₃ hydroxyalkyl. In certain embodiments, R² is C₄ hydroxyalkyl. In certain embodiments, R² is —(C₁₋₆ alkylene)-N(R⁸)(R⁹). In certain embodiments, R² is selected from the groups depicted in the compounds in Table 1 below.

As defined generally above, R⁵ is hydrogen, C₁₋₄ alkyl, C₂₋₄ aminoalkyl, —(C₁₋₄ alkylene)-(C₁₋₆ alkoxyl), or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₂₋₄ aminoalkyl. In certain embodiments, R⁵ is C₂ aminoalkyl. In certain embodiments, R⁵ is C₃ aminoalkyl. In certain embodiments, R⁵ is C₄ aminoalkyl. In certain embodiments, R⁵ is —(C₁₋₄ alkylene)-(C₁₋₆ alkoxyl). In certain embodiments, R⁵ is —CH₂CH₂OCH₃. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 1 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur. In certain embodiments, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰. In certain embodiments, R³ is C₁₋₆ alkyl. In certain embodiments, R³ is ethyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is halo. In certain embodiments, R³ is hydroxyl. In certain embodiments, R³ is C₁₋₆ haloalkyl. In certain embodiments, R³ is C₁₋₆ deuteroalkyl. In certain embodiments, R³ is C₁₋₆ deuteroalkoxyl, In certain embodiments, R³ is —O—C₃₋₇ cycloalkyl. In certain embodiments, R³ is cyano. In certain embodiments, R³ is —CO₂R¹⁰. In certain embodiments, R³ is —C(O)N(R⁸)(R⁹). In certain embodiments, R³ is —N(R⁸)C(O)R¹⁰. In certain embodiments, R³ is —S(O₂)R¹⁰. In certain embodiments, two occurrences of R³ are taken together with the intervening atoms to form a 5-7 membered ring containing 0, 1, or 2 heteroatoms independently selected from nitrogen, oxygen, and sulfur. In certain embodiments, R³ is selected from the groups depicted in the compounds in Table 1 below.

As defined generally above, R⁴ is hydrogen, halo, or C₁₋₄ alkyl. In certain embodiments, R⁴ is hydrogen. In certain embodiments, R⁴ is halo. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 1 below.

As defined generally above, R⁶ represents independently for each occurrence halo, hydroxyl, C₁₋₆ alkyl, C₁₋₆ haloalkyl, cyano, C₁₋₆ alkoxyl, C₃₋₇ cycloalkyl substituted with 0 or 1 occurrences of C₁₋₆ alkoxyl, C₃₋₇ halocycloalkyl, C₃₋₇ hydroxycycloalkyl, —O—C₃₋₇ cycloalkyl, nitro, —C(O)R⁷, —N(R⁸)(R⁹), oxo, C₁₋₄ hydroxyalkyl, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo. In certain embodiments, R⁶ is C₁₋₆ haloalkyl. In certain embodiments, R⁶ is —CF₃. In certain embodiments, R⁶ is C₃₋₇ cycloalkyl substituted with 0 or 1 occurrences of C₁₋₆ alkoxyl. In certain embodiments, R⁶ is C₃₋₇ cycloalkyl. In certain embodiments, R⁶ is cyclopropyl. In certain embodiments, R⁶ is C₃₋₇ cycloalkyl substituted with 1 occurrence of C₁₋₆ alkoxyl. In certain embodiments, R⁶ is C₃₋₇ cycloalkyl substituted with 1 occurrence of —OCH₃. In certain embodiments, R⁶ is C₃₋₇ halocycloalkyl. In certain embodiments, R⁶ is C₃₋₇ hydroxycycloalkyl. In certain embodiments, R⁶ is halo. In certain embodiments, R⁶ represents independently for each occurrence C₁₋₆ haloalkyl, —C(O)R⁷, or —C(O)N(R⁸)(R⁹). In certain embodiments, R⁶ is hydroxyl. In certain embodiments, R⁶ is C₁₋₆ alkyl. In certain embodiments, R⁶ is cyano. In certain embodiments, R⁶ is C₁₋₆ alkoxyl. In certain embodiments, R⁶ is —O—C₃₋₇ cycloalkyl. In certain embodiments, R⁶ is —C(O)R⁷. In certain embodiments, R⁶ is —N(R⁸)(R⁹). In certain embodiments, R⁶ is —N(CH₃)₂. In certain embodiments, R⁶ is oxo. In certain embodiments, R⁶ is C₁₋₄ hydroxyalkyl. In certain embodiments, R⁶ is C₁ hydroxyalkyl. In certain embodiments, R⁶ is C₂ hydroxyalkyl. In certain embodiments, R⁶ is C₃ hydroxyalkyl. In certain embodiments, R⁶ is C₄ hydroxyalkyl. In certain embodiments, R⁶ is —C(O)N(R⁸)(R⁹). In certain embodiments, R⁶ is —N(R⁸)C(O)R¹⁰. In certain embodiments, R⁶ is —S(O₂)R¹⁰. In certain embodiments, R⁶ is —S(O₂)N(R⁸)(R⁹). In certain embodiments, R⁶ is —N(R⁸)S(O₂)R¹⁰. In certain embodiments, R⁶ is a 3-7 membered saturated heterocyclyl containing 1 or 2 heteroatoms independently selected from oxygen and nitrogen, wherein the heterocyclyl is substituted with 0 or 1 halo. In certain embodiments, R⁶ is a 3-7 membered saturated heterocyclyl containing 1 heteroatom selected from oxygen and nitrogen, wherein the heterocyclyl is substituted with 0 or 1 halo. In certain embodiments, R⁶ is a 3-7 membered saturated heterocyclyl containing 2 heteroatoms independently selected from oxygen and nitrogen, wherein the heterocyclyl is substituted with 0 or 1 halo. In certaine embodiments, R⁶ is nitro. In certain embodiments, R⁶ is selected from the groups depicted in the compounds in Table 1 below.

As defined generally above, R⁷ is —OH, C₁₋₄ alkyl, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is —OH. In certain embodiments, R⁷ is C₁₋₄ alkyl. In certain embodiments, R⁷ is C₁ alkyl. In certain embodiments, R⁷ is C₂ alkyl. In certain embodiments, R⁷ is C₃ alkyl. In certain embodiments, R⁷ is C₄ alkyl. In certain embodiments, R⁷ is —O—(C₁₋₆ alkyl). In certain embodiments, R⁷ is —O—C₃₋₇ cycloalkyl. In certain embodiments, R⁷ is a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 4-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 5-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 6-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is selected from the groups depicted in the compounds in Table 1 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 1 below. In certain embodiments, R⁹ is selected from the groups depicted in the compounds in Table 1 below.

As defined generally above, R¹⁰ represents independently for each occurrence C₁₋₆ alkyl or —(C₀₋₅ alkylene)-C₃₋₇ cycloalkyl. In certain embodiments, R¹⁰ is C₁₋₆ alkyl. In certain embodiments, R¹⁰ is —(C₀₋₅ alkylene)-C₃₋₇ cycloalkyl. In certain embodiments, R¹⁰ is selected from the groups depicted in the compounds in Table 1 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ is halo. In certain embodiments, R¹¹ is hydroxyl. In certain embodiments, R¹¹ is C₁₋₆ alkyl. In certain embodiments, R¹¹ is C₁₋₆ haloalkyl. In certain embodiments, R¹¹ is C₁₋₆ alkoxyl. In certain embodiments, R¹¹ is C₃₋₇ cycloalkyl. In certain embodiments, R¹¹ is selected from the groups depicted in the compounds in Table 1 below.

As defined generally above, A¹ is a 5-6 membered heteroaryl containing 1, 2, 3, or 4 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered saturated heterocyclyl containing 1, 2 or 3 heteroatoms independently selected from oxygen, nitrogen, and sulfur, or —N(R²)-(5-6 membered saturated carbocylic ring substituted with n occurrences of R⁶), wherein the heteroaryl and heterocyclyl are substituted with n occurrences of R⁶. In certain embodiments, A¹ is pyrazolyl, imidazolyl, oxazolyl, isoxazolyl, isothiazolyl, pyrrolyl, or furanyl, each of which is substituted with n occurrences of R⁶. In certain embodiments, A¹ is pyrazolyl substituted with n occurrences of R⁶. In certain embodiments, A¹ is

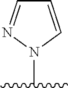 [SMILES: *n1cccn1]

substituted with n occurrences of R⁶. In certain embodiments, A¹ is

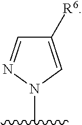

In certain embodiments, A¹ is a 5-membered heteroaryl containing 1 heteratom independently selected from oxygen, nitrogen, and sulfur, wherein the heteroaryl is substituted with n occurrences of R⁶. In certain embodiments, A¹ is a 5-membered heteroaryl containing 2 heteroatoms independently selected from oxygen, nitrogen, and sulfur, wherein the heteroaryl is substituted with n occurrences of R⁶. In certain embodiments, A¹ is a 5-membered heteroaryl containing 3 heteroatoms independently selected from oxygen, nitrogen, and sulfur, wherein the heteroaryl is substituted with n occurrences of R⁶. In certain embodiments, A¹ is a 5-membered heteroaryl containing 4 heteroatoms independently selected from oxygen, nitrogen, and sulfur, wherein the heteroaryl is substituted with n occurrences of R⁶. In certain embodiments, A¹ is a 6-membered heteroaryl containing 1 heteratom independently selected from oxygen, nitrogen, and sulfur, wherein the heteroaryl is substituted with n occurrences of R⁶. In certain embodiments, A¹ is a 6-membered heteroaryl containing 2 heteroatoms independently selected from oxygen, nitrogen, and sulfur, wherein the heteroaryl is substituted with n occurrences of R⁶. In certain embodiments, A¹ is a 6-membered heteroaryl containing 3 heteroatoms independently selected from oxygen, nitrogen, and sulfur, wherein the heteroaryl is substituted with n occurrences of R⁶. In certain embodiments, A¹ is a 6-membered heteroaryl containing 4 heteroatoms independently selected from oxygen, nitrogen, and sulfur, wherein the heteroaryl is substituted with n occurrences of R⁶.

In certain embodiments, A¹ is a 9-10 membered bicyclic heteroaryl containing 1, 2, or 3 heteroatoms independently selected from oxygen, nitrogen, and sulfur, wherein the heteroaryl is substituted with n occurrences of R⁶. In certain embodiments, A¹ is a 9-10 membered bicyclic heteroaryl containing 1 heteroatom independently selected from oxygen, nitrogen, and sulfur, wherein the heteroaryl is substituted with n occurrences of R⁶. In certain embodiments, A¹ is a 9-10 membered bicyclic heteroaryl containing 2 heteroatoms independently selected from oxygen, nitrogen, and sulfur, wherein the heteroaryl is substituted with n occurrences of R⁶. In certain embodiments, A¹ is a 9-10 membered bicyclic heteroaryl containing 3 heteroatoms independently selected from oxygen, nitrogen, and sulfur, wherein the heteroaryl is substituted with n occurrences of R⁶. In certain embodiments, A¹ is a 3-10 membered saturated heterocyclyl containing 1, 2 or 3 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 1 heteroatom selected from oxygen, nitrogen, and sulfur, wherein the heterocyclyl is substituted with n occurrences of R⁶. In certain embodiments, A¹ is a 3-10 membered saturated heterocyclyl containing 2 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 3 heteroatoms independently selected from oxygen, nitrogen, and sulfur, wherein the heterocyclyl is substituted with n occurrences of R⁶. In certain embodiments, A¹ is —N(R²)-(5-6 membered saturated carbocylic ring substituted with n occurrences of R⁶). In certain embodiments, A¹ is —N(CH₃)₂-(6 membered saturated carbocylic ring substituted with n occurrences of R⁶).

In certain embodiments, if A¹ is a 3-10 membered saturated heterocyclyl containing 1, 2 or 3 heteroatoms independently selected from oxygen, nitrogen, and sulfur, wherein the heterocyclyl is substituted with n occurrences of R⁶, then R⁶ is not hydroxyl.

In certain embodiments, A¹ is selected from the groups depicted in the compounds in Table 1 below.

As defined generally above, A² is a pyridinylene, pyridazinylene, pyrimidinylene, or phenylene. In certain embodiments, A² is a pyridinylene. In certain embodiments, A² is pyridazinylene or pyrimidinylene. In certain embodiments, A² is pyridazinylene. In certain embodiments, A² is pyrimidinylene. In certain embodiments, A² is phenylene. In certain embodiments, A² is selected from the groups depicted in the compounds in Table 1 below.

As defined generally above, A³ is

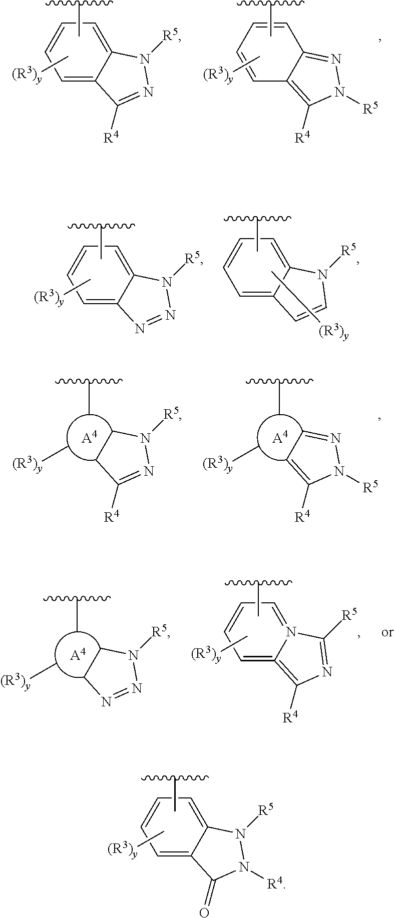

In certain embodiments, A³ is

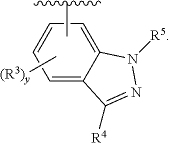

In certain embodiments, A³ is

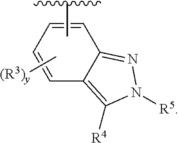

In certain embodiments, A³ is

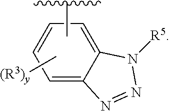

In certain embodiments, A³ is

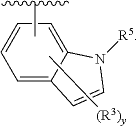

In certain embodiments, A³ is

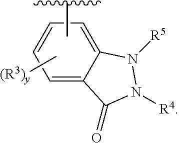

In certain embodiments, A³ is

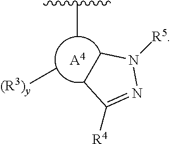

In certain embodiments, A³ is

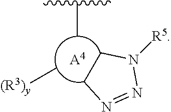

In certain embodiments, A³ is

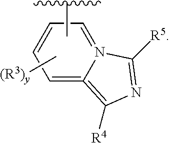

In certain embodiments, A³ is

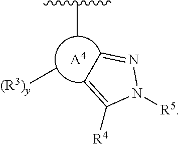

In certain embodiments, A³ is selected from the groups depicted in the compounds in Table 1 below.

As defined generally above, A⁴ is a 6-membered aromatic ring containing 1 nitrogen atom. In certain embodiments, A⁴ is selected from the groups depicted in the compounds in Table 1 below.

As defined generally above, y is 0, 1, or 2. In certain embodiments, y is 0. In certain embodiments, y is 1. In certain embodiments, y is 2. In certain embodiments, y is selected from the corresponding value in the groups depicted in the compounds in Table 1 below.

As defined generally above, m and x are independently 0, 1, or 2. In certain embodiments, x is 0. In certain embodiments, m is 0. In certain embodiments, m is 1. In certain embodiments, m is 2. In certain embodiments, x is 1. In certain embodiments, x is 2. In certain embodiments, m is selected from the corresponding value in the groups depicted in the compounds in Table 1 below. In certain embodiments, x is selected from the corresponding value in the groups depicted in the compounds in Table 1 below.

As defined generally above, n is 0, 1, 2, or 3. In certain embodiments, n is 0. In certain embodiments, n is 1. In certain embodiments, n is 2. In certain embodiments, n is 3. In certain embodiments, n is selected from the corresponding value in the groups depicted in the compounds in Table 1 below.

The description above describes multiple embodiments relating to compounds of Formula I. The patent application specifically contemplates all combinations of the embodiments.

In certain embodiments, the compound of Formula I is further defined by Formula Ia:

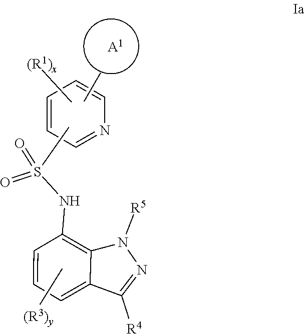

or a pharmaceutically acceptable salt thereof. In certain embodiments, the definition of variables R¹, R³, R⁴, R⁵, A¹, x, and y is one of the embodiments described above in connection with Formula I.

The description above describes multiple embodiments relating to compounds of Formula Ia. The patent application specifically contemplates all combinations of the embodiments.

In certain embodiments, the compound of Formula I is further defined by Formula Ib:

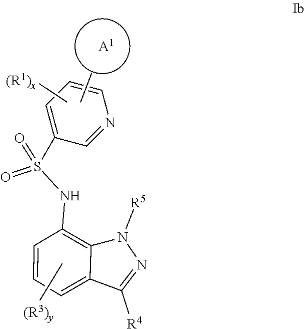

or a pharmaceutically acceptable salt thereof. In certain embodiments, the definition of variables R¹, R³, R⁴, R⁵, A¹, x, and y is one of the embodiments described above in connection with Formula I.

The description above describes multiple embodiments relating to compounds of Formula Ib. The patent application specifically contemplates all combinations of the embodiments.

In certain embodiments, the compound of Formula I is further defined by Formula Ic:

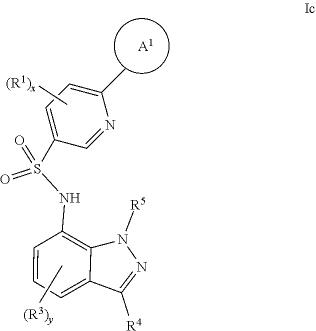

or a pharmaceutically acceptable salt thereof. In certain embodiments, the definition of variables R¹, R³, R⁴, R⁵, A¹, x, and y is one of the embodiments described above in connection with Formula I.

The description above describes multiple embodiments relating to compounds of Formula Ic. The patent application specifically contemplates all combinations of the embodiments.

In certain embodiments, the compound of Formula I is further defined by Formula Id:

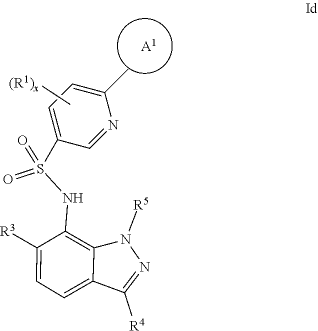

or a pharmaceutically acceptable salt thereof. In certain embodiments, the definition of variables R¹, R³, R⁴, R⁵, A¹, and x is one of the embodiments described above in connection with Formula I.

The description above describes multiple embodiments relating to compounds of Formula Id. The patent application specifically contemplates all combinations of the embodiments.

In certain embodiments, the compound is represented by Formula Ie:

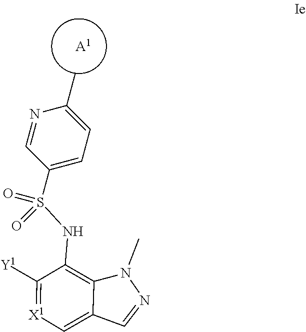

or a pharmaceutically acceptable salt thereof, wherein X¹ is nitrogen or —C(H)—; and Y¹ is hydrogen or —OCH₃.

In certain embodiments, the definition of variable A¹ is one of the embodiments described above in connection with Formula I. In certain of said embodiments, A¹ is selected from

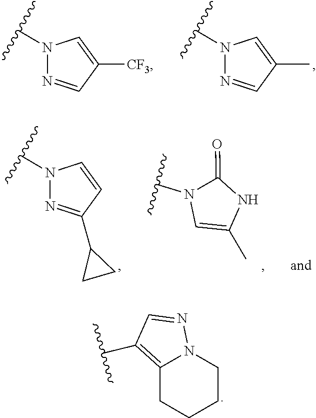

In certain of said embodiments, A¹ is

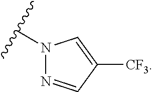 [SMILES: *n1cc(C(F)(F)F)cn1]

In certain of said embodiments, A¹ is N

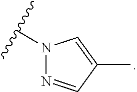 [SMILES: *n1cc(C)cn1]

In certain of said embodiments, A¹ is

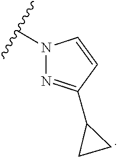 [SMILES: *n1ccc(C2CC2)n1]

In certain of said embodiments, A¹ is

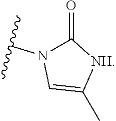 [SMILES: *n1cc(C)[nH]c1=O]

In certain of said embodiments, A¹ is

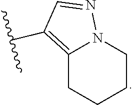 [SMILES: *c1cnn2c1CCCC2]

In certain embodiments, the compound is represented by Formula If:

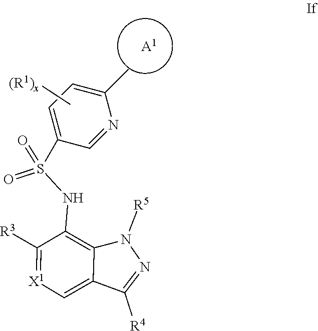

or a pharmaceutically acceptable salt thereof, wherein X¹ is nitrogen or —C(H)—, and R³ is hydrogen or —OCH₃. In certain embodiments, X¹ is nitrogen. In certain embodiments, X¹ is —C(H)—. In certain embodiments, R³ is hydrogen. In certain embodiments, R³ is —OCH₃.

In certain embodiments, the definition of variable A¹ is one of the embodiments described above in connection with Formula I. In certain of said embodiments, A¹ is selected from

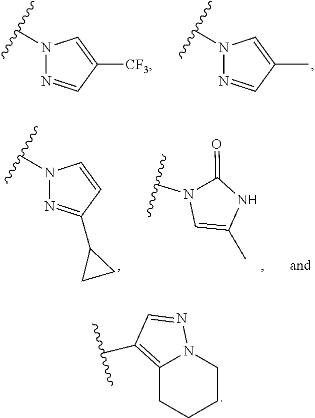

In certain of said embodiments, A¹ is

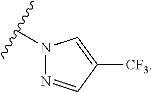 [SMILES: *n1cc(C(F)(F)F)cn1]

In certain of said embodiments, A¹ is N

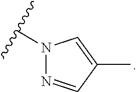 [SMILES: *n1cc(C)cn1]

In certain of said embodiments A¹ is

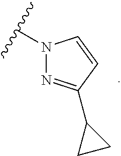 [SMILES: *n1ccc(C2CC2)n1]

In certain of said embodiments, A¹ is

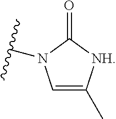 [SMILES: *n1cc(C)[nH]c1=O]

In certain of said embodiments, A¹ is

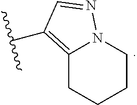 [SMILES: *c1cnn2c1CCCC2]

The description above describes multiple embodiments relating to compounds of Formula Ie. The patent application specifically contemplates all combinations of the embodiments.

### Part B:

Another of the invention provides a compound represented by Formula I-1:

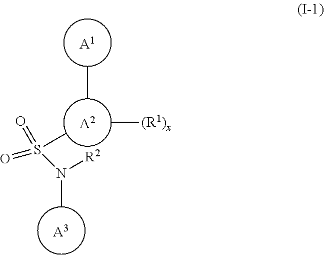

or a pharmaceutically acceptable salt thereof, wherein:
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, C₃₋₇ halocycloalkyl, C₃₋₇ hydroxycycloalkyl, —O—C₃₋₇ cycloalkyl, nitro, —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or —(C₀₋₅ alkylene)-C₃₋₇ cycloalkyl; R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2 or 3 heteroatoms independently selected from oxygen, nitrogen, and sulfur, wherein the heteroaryl and heterocyclyl are substituted with n occurrences of R⁶;
A² is a pyridinylene, pyridazinylene, pyrimidinylene, or phenylene;
A³ is

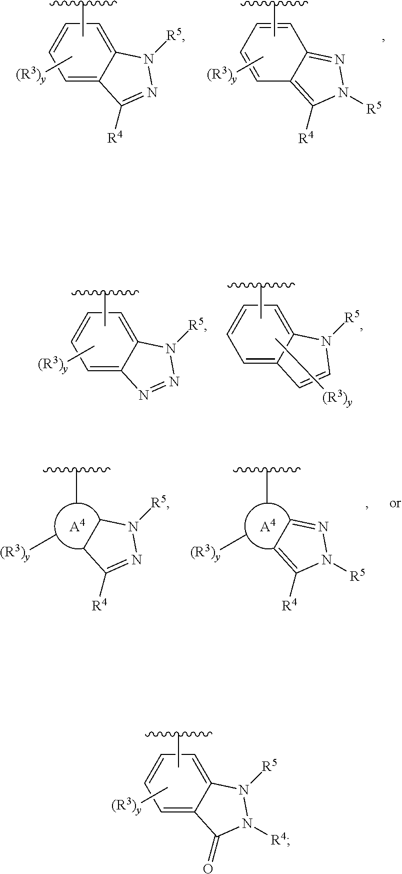

A⁴ is a 6-membered aromatic ring containing 1 nitrogen atom;
y is 0, 1, or 2; and
m, n, and x are independently 0, 1, or 2;
provided that if A² is phenylene, then y is 1 or 2 and at least one occurrence of R³ is C₁₋₆ alkoxyl, C₁₋₆ deuteroalkoxyl, or C₃₋₇ cycloalkyl.

The definitions of variables in Formula I-1 above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I-1.

As defined generally above, R¹ represents independently for each occurrence halo, C₁₋₄ alkyl, C₁₋₄ haloalkyl, or cyano. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is C₁₋₄ haloalkyl. In certain embodiments, R¹ is C₁ haloalkyl. In certain embodiments, R¹ is C₂ haloalkyl. In certain embodiments, R¹ is C₃ haloalkyl. In certain embodiments, R¹ is C₄ haloalkyl. In certain embodiments, R¹ is cyano. In certain embodiments, R¹ is selected from the groups depicted in the compounds in Table 1 below.

As defined generally above, R² is hydrogen, C₁₋₄ alkyl, C₂₋₄ hydroxyalkyl, or —(C₁₋₆ alkylene)-N(R⁸)(R⁹). In certain embodiments, R² is hydrogen. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is C₂₋₄ hydroxyalkyl. In certain embodiments, R² is C₂ hydroxyalkyl. In certain embodiments, R² is C₃ hydroxyalkyl. In certain embodiments, R² is C₄ hydroxyalkyl. In certain embodiments, R² is —(C₁₋₆ alkylene)-N(R⁸)(R⁹). In certain embodiments, R² is selected from the groups depicted in the compounds in Table 1 below.

As defined generally above, R⁵ is hydrogen, C₁₋₄ alkyl, or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 1 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur. In certain embodiments, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰. In certain embodiments, R³ is C₁₋₆ alkyl. In certain embodiments, R³ is ethyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is halo. In certain embodiments, R³ is hydroxyl. In certain embodiments, R³ is C₁₋₆ haloalkyl. In certain embodiments, R³ is C₁₋₆ deuteroalkyl. In certain embodiments, R³ is C₁₋₆ deuteroalkoxyl, In certain embodiments, R³ is —O—C₃₋₇ cycloalkyl. In certain embodiments, R³ is cyano. In certain embodiments, R³ is —CO₂R¹⁰. In certain embodiments, R³ is —C(O)N(R⁸)(R⁹). In certain embodiments, R³ is —N(R⁸)C(O)R¹⁰. In certain embodiments, R³ is —S(O₂)R¹⁰. In certain embodiments, two occurrences of R³ are taken together with the intervening atoms to form a 5-7 membered ring containing 0, 1, or 2 heteroatoms independently selected from nitrogen, oxygen, and sulfur. In certain embodiments, R³ is selected from the groups depicted in the compounds in Table 1 below.

As defined generally above, R⁴ is hydrogen, halo, or C₁₋₄ alkyl. In certain embodiments, R⁴ is hydrogen. In certain embodiments, R⁴ is halo. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 1 below.

As defined generally above, R⁶ represents independently for each occurrence halo, hydroxyl, C₁₋₆ alkyl, C₁₋₆ haloalkyl, cyano, C₁₋₆ alkoxyl, C₃₋₇ cycloalkyl, C₃₋₇halocycloalkyl, C₃₋₇ hydroxycycloalkyl, —O—C₃₋₇ cycloalkyl, nitro, —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo. In certain embodiments, R⁶ is C₁₋₆ haloalkyl. In certain embodiments, R⁶ is —CF₃. In certain embodiments, C₃₋₇ cycloalkyl. In certain embodiments, R⁶ is cyclopropyl. In certain embodiments, R⁶ is C₃₋₇ halocycloalkyl. In certain embodiments, R⁶ is C₃₋₇ hydroxycycloalkyl. In certain embodiments, R⁶ is halo. In certain embodiments, R⁶ represents independently for each occurrence C₁₋₆ haloalkyl, —C(O)R⁷, or —C(O)N(R⁸)(R⁹). In certain embodiments, R⁶ is hydroxyl. In certain embodiments, R⁶ is C₁₋₆ alkyl. In certain embodiments, R⁶ is cyano. In certain embodiments, R⁶ is C₁₋₆ alkoxyl. In certain embodiments, R⁶ is —O—C₃₋₇ cycloalkyl. In certain embodiments, R⁶ is —C(O)R⁷. In certain embodiments, R⁶ is —C(O)N(R⁸)(R⁹). In certain embodiments, R⁶ is —N(R⁸)C(O)R¹⁰. In certain embodiments, R⁶ is —S(O₂)R¹⁰. In certain embodiments, R⁶ is —S(O₂)N(R⁸)(R⁹). In certain embodiments, R⁶ is —N(R⁸)S(O₂)R¹⁰. In certain embodiments, R⁶ is a 3-7 membered saturated heterocyclyl containing 1 or 2 heteroatoms independently selected from oxygen and nitrogen, wherein the heterocyclyl is substituted with 0 or 1 halo. In certain embodiments, R⁶ is a 3-7 membered saturated heterocyclyl containing 1 heteroatom selected from oxygen and nitrogen, wherein the heterocyclyl is substituted with 0 or 1 halo. In certain embodiments, R⁶ is a 3-7 membered saturated heterocyclyl containing 2 heteroatoms independently selected from oxygen and nitrogen, wherein the heterocyclyl is substituted with 0 or 1 halo. In certaine embodiments, R⁶ is nitro. In certain embodiments, R⁶ is selected from the groups depicted in the compounds in Table 1 below.

As defined generally above, R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is —OH. In certain embodiments, R⁷ is —OH. In certain embodiments, R⁷ is —O—(C₁₋₆ alkyl). In certain embodiments, R⁷ is —O—C₃₋₇ cycloalkyl. In certain embodiments, R⁷ is a 4-6 membered saturated heterocyclyl containing 1 or 2 heteroatoms independently selected from oxygen, nitrogen, and sulfur, wherein the heterocyclyl is substituted with m occurrences of R¹¹—O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 4-membered saturated heterocyclyl containing 1 or 2 heteroatoms independently selected from oxygen, nitrogen, and sulfur, wherein the heterocyclyl is substituted with m occurrences of R¹¹—O—(C₁₋₆ alkyl), —O—C₃₋₇ cycloalkyl, or a 4-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 5-membered saturated heterocyclyl containing 1 or 2 heteroatoms independently selected from oxygen, nitrogen, and sulfur, wherein the heterocyclyl is substituted with m occurrences of R¹¹—O—(C₁₋₆ alkyl), —O—C₃₋₇ cycloalkyl, or a 5-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 6-membered saturated heterocyclyl containing 1 or 2 heteroatoms independently selected from oxygen, nitrogen, and sulfur, wherein the heterocyclyl is substituted with m occurrences of R¹¹—O—(C₁₋₆ alkyl), —O—C₃₋₇ cycloalkyl, or a 6-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is selected from the groups depicted in the compounds in Table 1 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 1 below. In certain embodiments, R⁹ is selected from the groups depicted in the compounds in Table 1 below.

As defined generally above, R¹⁰ represents independently for each occurrence C₁₋₆ alkyl or —(C₀₋₅ alkylene)-C₃₋₇ cycloalkyl. In certain embodiments, R¹⁰ is C₁₋₆ alkyl. In certain embodiments, R¹⁰ is —(C₀₋₅ alkylene)-C₃₋₇ cycloalkyl. In certain embodiments, R¹⁰ is selected from the groups depicted in the compounds in Table 1 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ is halo. In certain embodiments, R¹¹ is hydroxyl. In certain embodiments, R¹¹ is C₁₋₆ alkyl. In certain embodiments, R¹¹ is C₁₋₆ haloalkyl. In certain embodiments, R¹¹ is C₁₋₆ alkoxyl. In certain embodiments, R¹¹ is C₃₋₇ cycloalkyl. In certain embodiments, R¹¹ is selected from the groups depicted in the compounds in Table 1 below.

As defined generally above, A¹ is a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2 or 3 heteroatoms independently selected from oxygen, nitrogen, and sulfur, wherein the heteroaryl and heterocyclyl are substituted with n occurrences of R⁶. In certain embodiments, A¹ is pyrazolyl, imidazolyl, oxazolyl, isoxazolyl, isothiazolyl, pyrrolyl, or furanyl, each of which is substituted with n occurrences of R⁶. In certain embodiments, A¹ is pyrazolyl substituted with n occurrences of R⁶. In certain embodiments, A¹ is

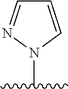 [SMILES: *n1cccn1]

substituted with n occurrences of R⁶. In certain embodiments, A¹ is

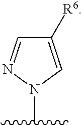

In certain embodiments, A¹ is a 5-membered heteroaryl containing 1 heteratom independently selected from oxygen, nitrogen, and sulfur, wherein the heteroaryl is substituted with n occurrences of R⁶. In certain embodiments, A¹ is a 5-membered heteroaryl containing 2 heteroatoms independently selected from oxygen, nitrogen, and sulfur, wherein the heteroaryl is substituted with n occurrences of R⁶. In certain embodiments, A¹ is a 5-membered heteroaryl containing 3 heteroatoms independently selected from oxygen, nitrogen, and sulfur, wherein the heteroaryl is substituted with n occurrences of R⁶.

In certain embodiments, A¹ is a 3-10 membered saturated heterocyclyl containing 1, 2 or 3 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 1 heteroatom selected from oxygen, nitrogen, and sulfur, wherein the heterocyclyl is substituted with n occurrences of R⁶. In certain embodiments, A¹ is a 3-10 membered saturated heterocyclyl containing 2 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 3 heteroatoms independently selected from oxygen, nitrogen, and sulfur, wherein the heterocyclyl is substituted with n occurrences of R⁶.

In certain embodiments, if A¹ is a 3-10 membered saturated heterocyclyl containing 3 heteroatoms independently selected from oxygen, nitrogen, and sulfur, wherein the heterocyclyl is substituted with n occurrences of R⁶.

In certain embodiments, if A¹ is a 3-10 membered saturated heterocyclyl containing 1, 2 or 3 heteroatoms independently selected from oxygen, nitrogen, and sulfur, wherein the heterocyclyl is substituted with n occurrences of R⁶, then R⁶ is not hydroxyl.

In certain embodiments, A¹ is selected from the groups depicted in the compounds in Table 1 below.

As defined generally above, A² is a pyridinylene, pyridazinylene, pyrimidinylene, or phenylene. In certain embodiments, A² is a pyridinylene. In certain embodiments, A² is pyridazinylene or pyrimidinylene. In certain embodiments, A² is pyridazinylene. In certain embodiments, A² is pyrimidinylene. In certain embodiments, A² is phenylene. In certain embodiments, A² is selected from the groups depicted in the compounds in Table 1 below.

As defined generally above, A³ is

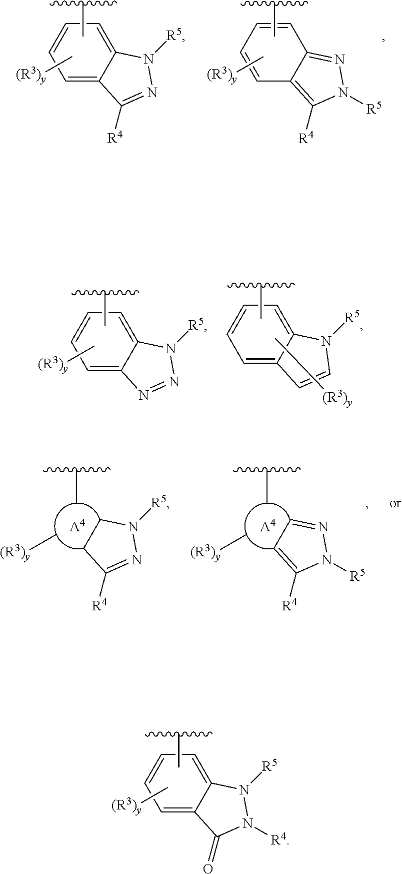

In certain embodiments, A³ is

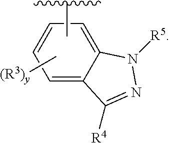

In certain embodiments, A³ is

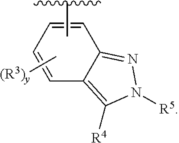

In certain embodiments, A³ is

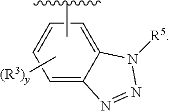

In certain embodiments, A³ is

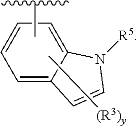

In certain embodiments, A³ is

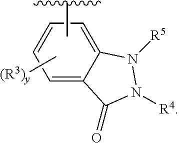

In certain embodiments, A³ is

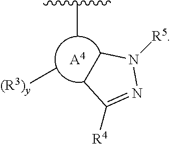

In certain embodiments, A³ is

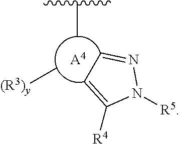

In certain embodiments, A³ is selected from the groups depicted in the compounds in Table 1 below.

As defined generally above, A⁴ is a 6-membered aromatic ring containing 1 nitrogen atom. In certain embodiments, A⁴ is selected from the groups depicted in the compounds in Table 1 below.

As defined generally above, y is 0, 1, or 2. In certain embodiments, y is 0. In certain embodiments, y is 1. In certain embodiments, y is 2. In certain embodiments, y is selected from the corresponding value in the groups depicted in the compounds in Table 1 below.

As defined generally above, m, n, and x are independently 0, 1, or 2. In certain embodiments, x is 0. In certain embodiments, n is 1. In certain embodiments, n is 2. In certain embodiments, m is 0. In certain embodiments, m is 1. In certain embodiments, m is 2. In certain embodiments, n is 0. In certain embodiments, x is 1. In certain embodiments, x is 2. In certain embodiments, m is selected from the corresponding value in the groups depicted in the compounds in Table 1 below. In certain embodiments, n is selected from the corresponding value in the groups depicted in the compounds in Table 1 below. In certain embodiments, x is selected from the corresponding value in the groups depicted in the compounds in Table 1 below.

The description above describes multiple embodiments relating to compounds of Formula I-1. The patent application specifically contemplates all combinations of the embodiments.

In certain embodiments, the compound of Formula I-1 is further defined by Formula Ia-1:

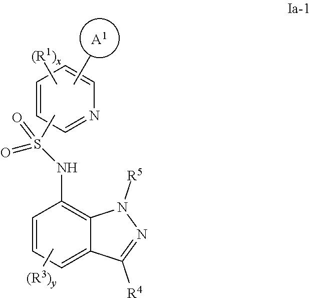

or a pharmaceutically acceptable salt thereof. In certain embodiments, the definition of variables R¹, R³, R⁴, R⁵, A¹, x, and y is one of the embodiments described above in connection with Formula I-1.

The description above describes multiple embodiments relating to compounds of Formula Ia-1. The patent application specifically contemplates all combinations of the embodiments.

In certain embodiments, the compound of Formula I-1 is further defined by Formula Ib-1:

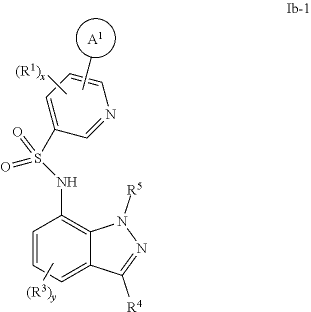

or a pharmaceutically acceptable salt thereof. In certain embodiments, the definition of variables R¹, R³, R⁴, R⁵, A¹, x, and y is one of the embodiments described above in connection with Formula I-1.

The description above describes multiple embodiments relating to compounds of Formula Ib-1. The patent application specifically contemplates all combinations of the embodiments.

In certain embodiments, the compound of Formula I-1 is further defined by Formula Ic-1:

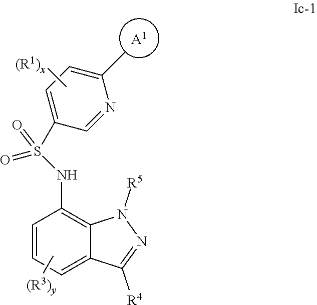

or a pharmaceutically acceptable salt thereof. In certain embodiments, the definition of variables R¹, R³, R⁴, R⁵, A¹, x, and y is one of the embodiments described above in connection with Formula I-1.

The description above describes multiple embodiments relating to compounds of Formula Ic-1. The patent application specifically contemplates all combinations of the embodiments.

In certain embodiments, the compound of Formula I-1 is further defined by Formula Id-1:

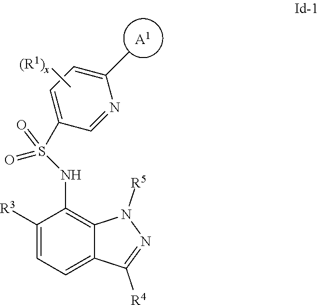

or a pharmaceutically acceptable salt thereof. In certain embodiments, the definition of variables R¹, R³, R⁴, R⁵, A¹, and x is one of the embodiments described above in connection with Formula I-1.

The description above describes multiple embodiments relating to compounds of Formula Id-1. The patent application specifically contemplates all combinations of the embodiments.

### Part C:

Another aspect of the invention provides a compound represented by Formula I-A:

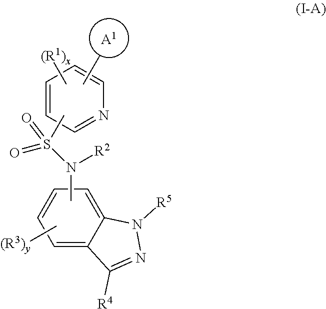

or a pharmaceutically acceptable salt thereof, wherein:
R¹ represents independently for each occurrence halo or C₁₋₄ alkyl;
R² and R⁵ are independently hydrogen or C₁₋₄ alkyl;
R³ represents independently for each occurrence halo, hydroxyl, C₁₋₆ alkyl, C₁₋₆ haloalkyl, C₁₋₆ alkoxyl, C₃₋₇ cycloalkyl, or —O—C₃₋₇ cycloalkyl;
R⁴ is hydrogen, halo, or C₁₋₄ alkyl;
R⁶ represents independently for each occurrence halo, hydroxyl, C₁₋₆ alkyl, C₁₋₆ haloalkyl, C₁₋₆ alkoxyl, C₃₋₇ cycloalkyl, —O—C₃₋₇ cycloalkyl, —C(O)R⁷, —C(O)N(R⁸)(R⁹), or —N(R⁸)C(O)R¹⁰;
R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ is C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, or 3 heteroatoms independently selected from oxygen, nitrogen, and sulfur, wherein the heteroaryl is substituted with n occurrences of R⁶;
y is 1 or 2; and
m, n, and x are independently 0, 1, or 2.

The definitions of variables in Formula I-A above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I-A.

As defined generally above, R¹ represents independently for each occurrence halo or C₁₋₄ alkyl. In certain embodiments, R¹ is halo. In certain embodiments, R¹ is F. In certain embodiments, R¹ is C₁. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is selected from the groups depicted in the compounds in Table 1 below.

As defined generally above, R² and R⁵ are independently hydrogen or C₁₋₄ alkyl. In certain embodiments, R² is hydrogen. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R² is selected from the groups depicted in the compounds in Table 1 below. In certain embodiments, R⁵ is selected from the groups depicted in the compounds in Table 1 below.

As defined generally above, R³ represents independently for each occurrence halo, hydroxyl, C₁₋₆ alkyl, C₁₋₆ haloalkyl, C₁₋₆ alkoxyl, C₃₋₇ cycloalkyl, or —O—C₃₋₇ cycloalkyl. In certain embodiments, R³ represents independently for each occurrence halo, hydroxyl, C₁₋₆ alkyl, C₁₋₆ haloalkyl. In certain embodiments, R³ is C₁₋₆ alkyl. In certain embodiments, R³ is ethyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is halo. In certain embodiments, R³ is hydroxyl. In certain embodiments, R³ is C₁₋₆ haloalkyl. In certain embodiments, R³ is —O—C₃₋₇ cycloalkyl. In certain embodiments, R³ is selected from the groups depicted in the compounds in Table 1 below.

As defined generally above, R⁴ is hydrogen, halo, or C₁₋₄ alkyl. In certain embodiments, R⁴ is hydrogen. In certain embodiments, R⁴ is halo. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 1 below.

As defined generally above, R⁶ represents independently for each occurrence halo, hydroxyl, C₁₋₆ alkyl, C₁₋₆ haloalkyl, C₁₋₆ alkoxyl, C₃₋₇ cycloalkyl, —O—C₃₋₇ cycloalkyl, —C(O)R⁷, —C(O)N(R⁸)(R⁹), or —N(R⁸)C(O)R¹⁰. In certain embodiments, R⁶ is C₁₋₆ haloalkyl. In certain embodiments, R⁶ is —CF₃. In certain embodiments, R⁶ is C₃₋₇ cycloalkyl. In certain embodiments, R⁶ is cyclopropyl. In certain embodiments, R⁶ is halo. In certain embodiments, R⁶ represents independently for each occurrence C₁₋₆ haloalkyl, —C(O)R⁷, or —C(O)N(R⁸)(R⁹). In certain embodiments, R⁶ is hydroxyl. In certain embodiments, R⁶ is C₁₋₆ alkyl. In certain embodiments, R⁶ is C₁₋₆ alkoxyl. In certain embodiments, R⁶ is —O—C₃₋₇ cycloalkyl. In certain embodiments, R⁶ is —C(O)R⁷. In certain embodiments, R⁶ is —C(O)N(R⁸)(R⁹). In certain embodiments, R⁶ is —N(R⁸)C(O)R¹⁰. In certain embodiments, R⁶ is selected from the groups depicted in the compounds in Table 1 below.

As defined generally above, R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is —OH. In certain embodiments, R⁷ is —O—(C₁₋₆ alkyl). In certain embodiments, R⁷ is —O—C₃₋₇ cycloalkyl. In certain embodiments, R⁷ is a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 4-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 5-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 6-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is selected from the groups depicted in the compounds in Table 1 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 1 below. In certain embodiments, R⁹ is selected from the groups depicted in the compounds in Table 1 below.

As defined generally above, R¹⁰ is C₁₋₆ alkyl or —(C₀₋₅ alkylene)-C₃₋₇ cycloalkyl. In certain embodiments, R¹⁰ is C₁₋₆ alkyl. In certain embodiments, R¹⁰ is C₁ alkyl. In certain embodiments, R¹⁰ is C₂ alkyl. In certain embodiments, R¹⁰ is C₃ alkyl. In certain embodiments, R¹⁰ is C₄ alkyl. In certain embodiments, R¹⁰ is C₅ alkyl. In certain embodiments, R¹⁰ is C₆ alkyl. In certain embodiments, R¹⁰ is —(C₀₋₅ alkylene)-C₃₋₇ cycloalkyl. In certain embodiments, R¹⁰ is selected from the groups depicted in the compounds in Table 1 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ is halo. In certain embodiments, R¹¹ is hydroxyl. In certain embodiments, R¹¹ is C₁₋₆ alkyl. In certain embodiments, R¹¹ is C₁₋₆ haloalkyl. In certain embodiments, R¹¹ is C₁₋₆ alkoxyl. In certain embodiments, R¹¹ is C₃₋₇ cycloalkyl. In certain embodiments, R¹¹ is selected from the groups depicted in the compounds in Table 1 below.

As defined generally above, A¹ is a 5-6 membered heteroaryl containing 1, 2, or 3 heteroatoms independently selected from oxygen, nitrogen, and sulfur, wherein the heteroaryl is substituted with n occurrences of R⁶. In certain embodiments, A¹ is pyrazolyl, imidazolyl, oxazolyl, isoxazolyl, isothiazolyl, pyrrolyl, or furanyl, each of which is substituted with n occurrences of R⁶. In certain embodiments, A¹ is pyrazolyl substituted with n occurrences of R⁶. In certain embodiments, A¹ is

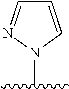 [SMILES: *n1cccn1]

substituted with n occurrences of R⁶. In certain embodiments, A¹ is

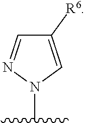

In certain embodiments, A¹ is a 5-membered heteroaryl containing 1 heteratom independently selected from oxygen, nitrogen, and sulfur, wherein the heteroaryl is substituted with n occurrences of R⁶. In certain embodiments, A¹ is a 5-membered heteroaryl containing 2 heteroatoms independently selected from oxygen, nitrogen, and sulfur, wherein the heteroaryl is substituted with n occurrences of R⁶. In certain embodiments, A¹ is a 5-membered heteroaryl containing 3 heteroatoms independently selected from oxygen, nitrogen, and sulfur, wherein the heteroaryl is substituted with n occurrences of R⁶. In certain embodiments, A¹ is selected from the groups depicted in the compounds in Table 1 below.

As defined generally above, y is 1 or 2. In certain embodiments, y is 1. In certain embodiments, y is 2. In certain embodiments, y is selected from the corresponding value in the groups depicted in the compounds in Table 1 below.

As defined generally above, m, n, and x are independently 0, 1, or 2. In certain embodiments, x is 0. In certain embodiments, n is 1. In certain embodiments, n is 2. In certain embodiments, m is 0. In certain embodiments, m is 1. In certain embodiments, m is 2. In certain embodiments, n is 0. In certain embodiments, x is 1. In certain embodiments, x is 2. In certain embodiments, m is selected from the corresponding value in the groups depicted in the compounds in Table 1 below. In certain embodiments, n is selected from the corresponding value in the groups depicted in the compounds in Table 1 below. In certain embodiments, x is selected from the corresponding value in the groups depicted in the compounds in Table 1 below.

The description above describes multiple embodiments relating to compounds of Formula I-A. The patent application specifically contemplates all combinations of the embodiments.

### Part D:

Another aspect of the invention provides a compound represented by Formula I-B:

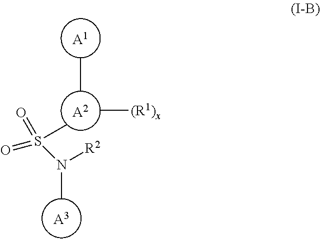

or a pharmaceutically acceptable salt thereof, wherein:
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, C₂₋₄ aminoalkyl, —(C₁₋₄ alkylene)-(C₁₋₆ alkoxyl), or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl substituted with 0 or 1 occurrences of C₁₋₆ alkoxyl, C₃₋₇ halocycloalkyl, C₃₋₇ hydroxycycloalkyl, —O—C₃₋₇ cycloalkyl, nitro, —C(O)R⁷, —N(R⁸)(R⁹), oxo, C₁₋₄ hydroxyalkyl, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, C₁₋₄ alkyl, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, 3, or 4 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered saturated heterocyclyl containing 1, 2 or 3 heteroatoms independently selected from oxygen, nitrogen, and sulfur, or —N(R²)-(5-6 membered saturated carbocylic ring substituted with n occurrences of R⁶), wherein the heteroaryl and heterocyclyl are substituted with n occurrences of R⁶;
A² is a pyridinylene, pyridazinylene, pyrimidinylene, or phenylene;
A³ is

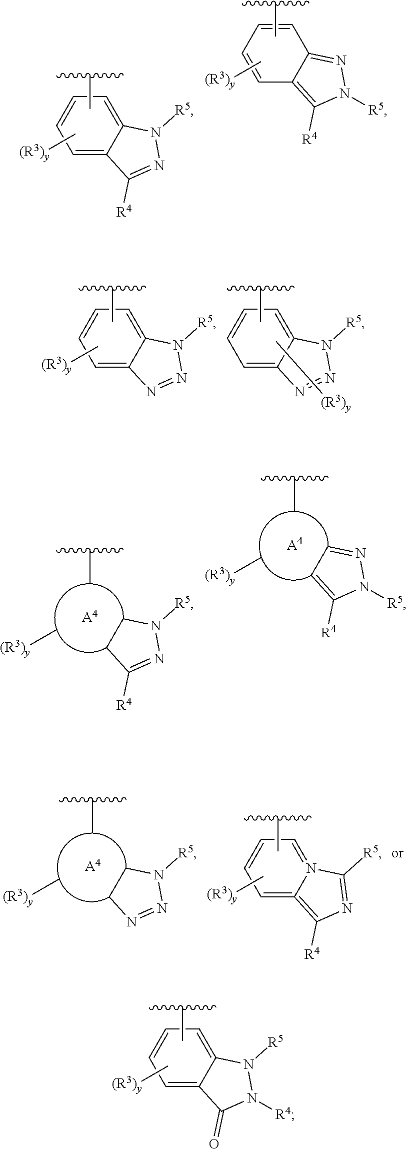

A⁴ is a 6-membered aromatic ring containing 1 nitrogen atom;
y is 0, 1, or 2;
m and x are independently 0, 1, or 2; and
n is 0, 1, 2, or 3;
provided that if A² is phenylene, then y is 1 or 2 and at least one occurrence of R³ is C₁₋₆ alkoxyl, C₁₋₆ deuteroalkoxyl, or C₃₋₇ cycloalkyl.

The definitions of variables in Formula I-B above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I-B.

As defined generally above, R¹ represents independently for each occurrence halo, C₁₋₄ alkyl, C₁₋₄ haloalkyl, or cyano. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is C₁₋₄ haloalkyl. In certain embodiments, R¹ is C₁ haloalkyl. In certain embodiments, R¹ is C₂ haloalkyl. In certain embodiments, R¹ is C₃ haloalkyl. In certain embodiments, R¹ is C₄ haloalkyl. In certain embodiments, R¹ is cyano. In certain embodiments, R¹ is selected from the groups depicted in the compounds in Table 1 below.

As defined generally above, R² is hydrogen, C₁₋₄ alkyl, C₂₋₄ hydroxyalkyl, or —(C₁₋₆ alkylene)-N(R⁸)(R⁹). In certain embodiments, R² is hydrogen. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is C₂₋₄ hydroxyalkyl. In certain embodiments, R² is C₂ hydroxyalkyl. In certain embodiments, R² is C₃ hydroxyalkyl. In certain embodiments, R² is C₄ hydroxyalkyl. In certain embodiments, R² is —(C₁₋₆ alkylene)-N(R⁸)(R⁹). In certain embodiments, R² is selected from the groups depicted in the compounds in Table 1 below.

As defined generally above, R⁵ is hydrogen, C₁₋₄ alkyl, C₂₋₄ aminoalkyl, —(C₁₋₄ alkylene)-(C₁₋₆ alkoxyl), or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₂₋₄ aminoalkyl. In certain embodiments, R⁵ is C₂ aminoalkyl. In certain embodiments, R⁵ is C₃ aminoalkyl. In certain embodiments, R⁵ is C₄ aminoalkyl. In certain embodiments, R⁵ is —(C₁₋₄ alkylene)-(C₁₋₆ alkoxyl). In certain embodiments, R⁵ is —CH₂CH₂OCH₃. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 1 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur. In certain embodiments, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰. In certain embodiments, R³ is C₁₋₆ alkyl. In certain embodiments, R³ is ethyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is halo. In certain embodiments, R³ is hydroxyl. In certain embodiments, R³ is C₁₋₆ haloalkyl. In certain embodiments, R³ is C₁₋₆ deuteroalkyl. In certain embodiments, R³ is C₁₋₆ deuteroalkoxyl, In certain embodiments, R³ is —O—C₃₋₇ cycloalkyl. In certain embodiments, R³ is cyano. In certain embodiments, R³ is —CO₂R¹⁰. In certain embodiments, R³ is —C(O)N(R⁸)(R⁹). In certain embodiments, R³ is —N(R⁸)C(O)R¹⁰. In certain embodiments, R³ is —S(O₂)R¹⁰. In certain embodiments, two occurrences of R³ are taken together with the intervening atoms to form a 5-7 membered ring containing 0, 1, or 2 heteroatoms independently selected from nitrogen, oxygen, and sulfur. In certain embodiments, R³ is selected from the groups depicted in the compounds in Table 1 below.

As defined generally above, R⁴ is hydrogen, halo, or C₁₋₄ alkyl. In certain embodiments, R⁴ is hydrogen. In certain embodiments, R⁴ is halo. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 1 below.

As defined generally above, R⁶ represents independently for each occurrence halo, hydroxyl, C₁₋₆ alkyl, C₁₋₆ haloalkyl, cyano, C₁₋₆ alkoxyl, C₃₋₇ cycloalkyl substituted with 0 or 1 occurrences of C₁₋₆ alkoxyl, C₃₋₇ halocycloalkyl, C₃₋₇ hydroxycycloalkyl, —O—C₃₋₇ cycloalkyl, nitro, —C(O)R⁷, —N(R⁸)(R⁹), oxo, C₁₋₄ hydroxyalkyl, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo. In certain embodiments, R⁶ is C₁₋₆ haloalkyl. In certain embodiments, R⁶ is —CF₃. In certain embodiments, R⁶ is C₃₋₇ cycloalkyl substituted with 0 or 1 occurrences of C₁₋₆ alkoxyl. In certain embodiments, R⁶ is C₃₋₇ cycloalkyl. In certain embodiments, R⁶ is cyclopropyl. In certain embodiments, R⁶ is C₃₋₇ cycloalkyl substituted with 1 occurrence of C₁₋₆ alkoxyl. In certain embodiments, R⁶ is C₃₋₇ cycloalkyl substituted with 1 occurrence of —OCH₃. In certain embodiments, R⁶ is C₃₋₇ halocycloalkyl. In certain embodiments, R⁶ is C₃₋₇ hydroxycycloalkyl. In certain embodiments, R⁶ is halo. In certain embodiments, R⁶ represents independently for each occurrence C₁₋₆ haloalkyl, —C(O)R⁷, or —C(O)N(R⁸)(R⁹). In certain embodiments, R⁶ is hydroxyl. In certain embodiments, R⁶ is C₁₋₆ alkyl. In certain embodiments, R⁶ is cyano. In certain embodiments, R⁶ is C₁₋₆ alkoxyl. In certain embodiments, R⁶ is —O—C₃₋₇ cycloalkyl. In certain embodiments, R⁶ is —C(O)R⁷. In certain embodiments, R⁶ is —N(R⁸)(R⁹). In certain embodiments, R⁶ is —N(CH₃)₂. In certain embodiments, R⁶ is oxo. In certain embodiments, R⁶ is C₁₋₄ hydroxyalkyl. In certain embodiments, R⁶ is C₁ hydroxyalkyl. In certain embodiments, R⁶ is C₂ hydroxyalkyl. In certain embodiments, R⁶ is C₃ hydroxyalkyl. In certain embodiments, R⁶ is C₄ hydroxyalkyl. In certain embodiments, R⁶ is —C(O)N(R⁸)(R⁹). In certain embodiments, R⁶ is —N(R⁸)C(O)R¹⁰. In certain embodiments, R⁶ is —S(O₂)R¹⁰. In certain embodiments, R⁶ is —S(O₂)N(R⁸)(R⁹). In certain embodiments, R⁶ is —N(R⁸)S(O₂)R¹⁰. In certain embodiments, R⁶ is a 3-7 membered saturated heterocyclyl containing 1 or 2 heteroatoms independently selected from oxygen and nitrogen, wherein the heterocyclyl is substituted with 0 or 1 halo. In certain embodiments, R⁶ is a 3-7 membered saturated heterocyclyl containing 1 heteroatom selected from oxygen and nitrogen, wherein the heterocyclyl is substituted with 0 or 1 halo. In certain embodiments, R⁶ is a 3-7 membered saturated heterocyclyl containing 2 heteroatoms independently selected from oxygen and nitrogen, wherein the heterocyclyl is substituted with 0 or 1 halo. In certaine embodiments, R⁶ is nitro. In certain embodiments, R⁶ is selected from the groups depicted in the compounds in Table 1 below.

As defined generally above, R⁷ is —OH, C₁₋₄ alkyl, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is —OH. In certain embodiments, R⁷ is C₁₋₄ alkyl. In certain embodiments, R⁷ is C₁ alkyl. In certain embodiments, R⁷ is C₂ alkyl. In certain embodiments, R⁷ is C₃ alkyl. In certain embodiments, R⁷ is C₄ alkyl. In certain embodiments, R⁷ is —O—(C₁₋₆ alkyl). In certain embodiments, R⁷ is —O—C₃₋₇ cycloalkyl. In certain embodiments, R⁷ is a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 4-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 5-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 6-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is selected from the groups depicted in the compounds in Table 1 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 1 below. In certain embodiments, R⁹ is selected from the groups depicted in the compounds in Table 1 below.

As defined generally above, R¹⁰ represents independently for each occurrence C₁₋₆ alkyl or —(C₀₋₅ alkylene)-C₃₋₇ cycloalkyl. In certain embodiments, R¹⁰ is C₁₋₆ alkyl. In certain embodiments, R¹⁰ is —(C₀₋₅ alkylene)-C₃₋₇ cycloalkyl. In certain embodiments, R¹⁰ is selected from the groups depicted in the compounds in Table 1 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ is halo. In certain embodiments, R¹¹ is hydroxyl. In certain embodiments, R¹¹ is C₁₋₆ alkyl. In certain embodiments, R¹¹ is C₁₋₆ haloalkyl. In certain embodiments, R¹¹ is C₁₋₆ alkoxyl. In certain embodiments, R¹¹ is C₃₋₇ cycloalkyl. In certain embodiments, R¹¹ is selected from the groups depicted in the compounds in Table 1 below.

As defined generally above, A¹ is a 5-6 membered heteroaryl containing 1, 2, 3, or 4 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered saturated heterocyclyl containing 1, 2 or 3 heteroatoms independently selected from oxygen, nitrogen, and sulfur, or —N(R²)-(5-6 membered saturated carbocylic ring substituted with n occurrences of R⁶), wherein the heteroaryl and heterocyclyl are substituted with n occurrences of R⁶. In certain embodiments, A¹ is pyrazolyl, imidazolyl, oxazolyl, isoxazolyl, isothiazolyl, pyrrolyl, or furanyl, each of which is substituted with n occurrences of R⁶. In certain embodiments, A¹ is pyrazolyl substituted with n occurrences of R⁶. In certain embodiments, A¹ is

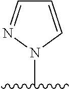 [SMILES: *n1cccn1]

substituted with n occurrences of R⁶. In certain embodiments, A¹ is

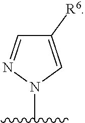

In certain embodiments, A¹ is a 5-membered heteroaryl containing 1 heteratom independently selected from oxygen, nitrogen, and sulfur, wherein the heteroaryl is substituted with n occurrences of R⁶. In certain embodiments, A¹ is a 5-membered heteroaryl containing 2 heteroatoms independently selected from oxygen, nitrogen, and sulfur, wherein the heteroaryl is substituted with n occurrences of R⁶. In certain embodiments, A¹ is a 5-membered heteroaryl containing 3 heteroatoms independently selected from oxygen, nitrogen, and sulfur, wherein the heteroaryl is substituted with n occurrences of R⁶. In certain embodiments, A¹ is a 5-membered heteroaryl containing 4 heteroatoms independently selected from oxygen, nitrogen, and sulfur, wherein the heteroaryl is substituted with n occurrences of R⁶. In certain embodiments, A¹ is a 6-membered heteroaryl containing 1 heteratom independently selected from oxygen, nitrogen, and sulfur, wherein the heteroaryl is substituted with n occurrences of R⁶. In certain embodiments, A¹ is a 6-membered heteroaryl containing 2 heteroatoms independently selected from oxygen, nitrogen, and sulfur, wherein the heteroaryl is substituted with n occurrences of R⁶. In certain embodiments, A¹ is a 6-membered heteroaryl containing 3 heteroatoms independently selected from oxygen, nitrogen, and sulfur, wherein the heteroaryl is substituted with n occurrences of R⁶. In certain embodiments, A¹ is a 6-membered heteroaryl containing 4 heteroatoms independently selected from oxygen, nitrogen, and sulfur, wherein the heteroaryl is substituted with n occurrences of R⁶.

In certain embodiments, A¹ is a 9-10 membered bicyclic heteroaryl containing 1, 2, or 3 heteroatoms independently selected from oxygen, nitrogen, and sulfur, wherein the heteroaryl is substituted with n occurrences of R⁶. In certain embodiments, A¹ is a 9-10 membered bicyclic heteroaryl containing 1 heteroatom independently selected from oxygen, nitrogen, and sulfur, wherein the heteroaryl is substituted with n occurrences of R⁶. In certain embodiments, A¹ is a 9-10 membered bicyclic heteroaryl containing 2 heteroatoms independently selected from oxygen, nitrogen, and sulfur, wherein the heteroaryl is substituted with n occurrences of R⁶. In certain embodiments, A¹ is a 9-10 membered bicyclic heteroaryl containing 3 heteroatoms independently selected from oxygen, nitrogen, and sulfur, wherein the heteroaryl is substituted with n occurrences of R⁶. In certain embodiments, A¹ is a 3-10 membered saturated heterocyclyl containing 1, 2 or 3 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 1 heteroatom selected from oxygen, nitrogen, and sulfur, wherein the heterocyclyl is substituted with n occurrences of R⁶. In certain embodiments, A¹ is a 3-10 membered saturated heterocyclyl containing 2 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 3 heteroatoms independently selected from oxygen, nitrogen, and sulfur, wherein the heterocyclyl is substituted with n occurrences of R⁶. In certain embodiments, A¹ is —N(R²)-(5-6 membered saturated carbocylic ring substituted with n occurrences of R⁶). In certain embodiments, A¹ is —N(CH₃)₂-(6 membered saturated carbocylic ring substituted with n occurrences of R⁶).

In certain embodiments, if A¹ is a 3-10 membered saturated heterocyclyl containing 1, 2 or 3 heteroatoms independently selected from oxygen, nitrogen, and sulfur, wherein the heterocyclyl is substituted with n occurrences of R⁶, then R⁶ is not hydroxyl.

In certain embodiments, A¹ is selected from the groups depicted in the compounds in Table 1 below.

As defined generally above, A² is a pyridinylene, pyridazinylene, pyrimidinylene, or phenylene. In certain embodiments, A² is a pyridinylene. In certain embodiments, A² is pyridazinylene or pyrimidinylene. In certain embodiments, A² is pyridazinylene. In certain embodiments, A² is pyrimidinylene. In certain embodiments, A² is phenylene. In certain embodiments, A² is selected from the groups depicted in the compounds in Table 1 below.

As defined generally above, A³ is

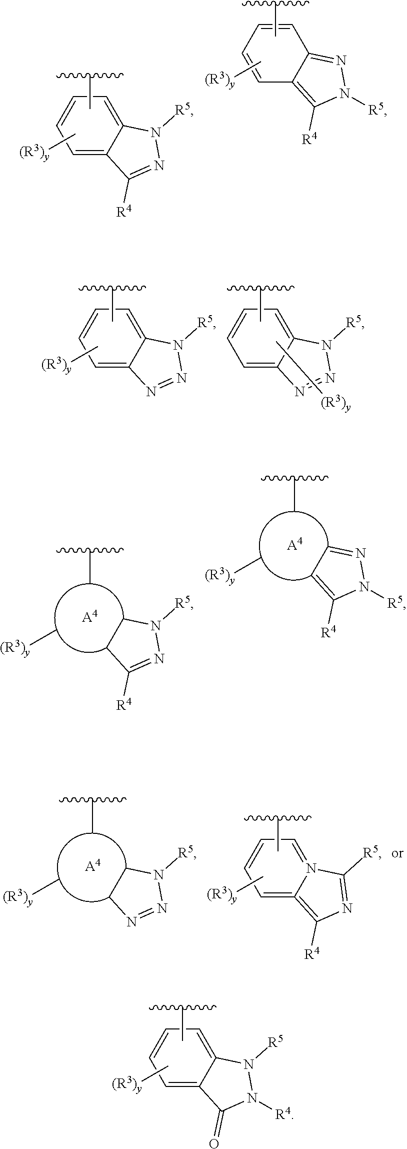

In certain embodiments, A³ is

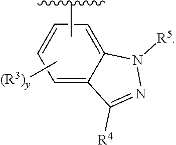

In certain embodiments, A³ is

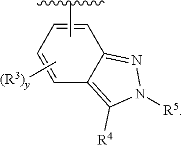

In certain embodiments, A³ is

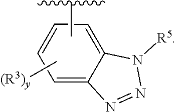

In certain embodiments, A³ is

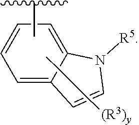

In certain embodiments, A³ is

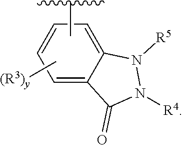

In certain embodiments, A³ is

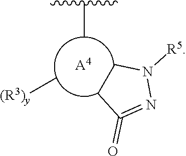

In certain embodiments A³ is

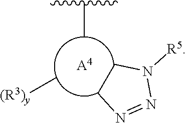

In certain embodiments, A³ is

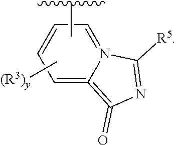

In certain embodiments, A³ is

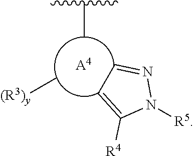

In certain embodiments, A³ is selected from the groups depicted in the compounds in Table 1 below.

As defined generally above, A⁴ is a 6-membered aromatic ring containing 1 nitrogen atom. In certain embodiments, A⁴ is selected from the groups depicted in the compounds in Table 1 below.

As defined generally above, y is 0, 1, or 2. In certain embodiments, y is 0. In certain embodiments, y is 1. In certain embodiments, y is 2. In certain embodiments, y is selected from the corresponding value in the groups depicted in the compounds in Table 1 below.

As defined generally above, m and x are independently 0, 1, or 2. In certain embodiments, x is 0. In certain embodiments, m is 0. In certain embodiments, m is 1. In certain embodiments, m is 2. In certain embodiments, x is 1. In certain embodiments, x is 2. In certain embodiments, m is selected from the corresponding value in the groups depicted in the compounds in Table 1 below. In certain embodiments, x is selected from the corresponding value in the groups depicted in the compounds in Table 1 below.

As defined generally above, n is 0, 1, 2, or 3. In certain embodiments, n is 0. In certain embodiments, n is 1. In certain embodiments, n is 2. In certain embodiments, n is 3. In certain embodiments, n is selected from the corresponding value in the groups depicted in the compounds in Table 1 below.

The description above describes multiple embodiments relating to compounds of Formula I-B. The patent application specifically contemplates all combinations of the embodiments.

### Part E:

Another aspect of the invention provides a compound represented by Formula I-C:

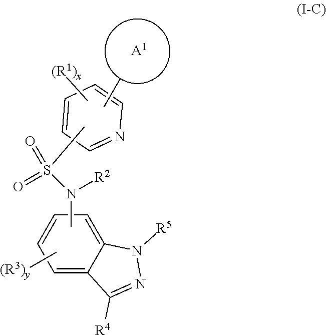

or a pharmaceutically acceptable salt thereof, wherein:
R¹ represents independently for each occurrence halo or C₁₋₄ alkyl;
R² is hydrogen or C₁₋₄ alkyl;
R⁵ is hydrogen, C₁₋₄ alkyl, C₂₋₄ aminoalkyl, —(C₁₋₄ alkylene)-(C₁₋₆ alkoxyl), or C₁₋₄ deuteroalkyl;
R³ represents independently for each occurrence halo, hydroxyl, C₁₋₆ alkyl, C₁₋₆ haloalkyl, C₁₋₆ alkoxyl, C₃₋₇ cycloalkyl, or —O—C₃₋₇ cycloalkyl;
R⁴ is hydrogen, halo, or C₁₋₄ alkyl;
R⁶ represents independently for each occurrence halo, hydroxyl, C₁₋₆ alkyl, C₁₋₆ haloalkyl, C₁₋₆ alkoxyl, C₃₋₇ cycloalkyl substituted with 0 or 1 occurrences of C₁₋₆ alkoxyl, —O—C₃₋₇ cycloalkyl, —C(O)R⁷, —N(R⁸)(R⁹), oxo, C₁₋₄ hydroxyalkyl, —C(O)N(R⁸)(R⁹), or —N(R⁸)C(O)R¹⁰;
R⁷ is —OH, C₁₋₄ alkyl, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ is C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, 3, or 4 heteroatoms independently selected from oxygen, nitrogen, and sulfur, wherein the heteroaryl is substituted with n occurrences of R⁶;
y is 1 or 2;
m and x are independently 0, 1, or 2; and
n is 0, 1, 2, or 3.

The definitions of variables in Formula I-C above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I-C.

As defined generally above, R¹ represents independently for each occurrence halo or C₁₋₄ alkyl. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is selected from the groups depicted in the compounds in Table 1 below.

As defined generally above, R² is hydrogen or C₁₋₄ alkyl. In certain embodiments, R² is hydrogen. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is selected from the groups depicted in the compounds in Table 1 below.

As defined generally above, R⁵ is hydrogen, C₁₋₄ alkyl, C₂₋₄ aminoalkyl, —(C₁₋₄ alkylene)-(C₁₋₆ alkoxyl), or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₂₋₄ aminoalkyl. In certain embodiments, R⁵ is C₂ aminoalkyl. In certain embodiments, R⁵ is C₃ aminoalkyl. In certain embodiments, R⁵ is C₄ aminoalkyl. In certain embodiments, R⁵ is —(C₁₋₄ alkylene)-(C₁₋₆ alkoxyl). In certain embodiments, R⁵ is —CH₂CH₂OCH₃. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 1 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, or —O—C₃₋₇ cycloalkyl. In certain embodiments, R³ is C₁₋₆ alkyl. In certain embodiments, R³ is ethyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is halo. In certain embodiments, R³ is hydroxyl. In certain embodiments, R³ is C₁₋₆ haloalkyl. In certain embodiments, R³ is C₁₋₆ deuteroalkyl. In certain embodiments, R³ is C₁₋₆ deuteroalkoxyl, In certain embodiments, R³ is —O—C₃₋₇ cycloalkyl. In certain embodiments, R³ is selected from the groups depicted in the compounds in Table 1 below.

As defined generally above, R⁴ is hydrogen, halo, or C₁₋₄ alkyl. In certain embodiments, R⁴ is hydrogen. In certain embodiments, R⁴ is halo. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 1 below.

As defined generally above, R⁶ represents independently for each occurrence halo, hydroxyl, C₁₋₆ alkyl, C₁₋₆ haloalkyl, C₁₋₆ alkoxyl, C₃₋₇ cycloalkyl substituted with 0 or 1 occurrences of C₁₋₆ alkoxyl, —O—C₃₋₇ cycloalkyl, —C(O)R⁷, —N(R⁸)(R⁹), oxo, C₁₋₄ hydroxyalkyl, —C(O)N(R⁸)(R⁹), or —N(R⁸)C(O)R¹⁰. In certain embodiments, R⁶ is C₁₋₆ haloalkyl. In certain embodiments, R⁶ is —CF₃. In certain embodiments, R⁶ is C₃₋₇ cycloalkyl substituted with 0 or 1 occurrences of C₁₋₆ alkoxyl. In certain embodiments, R⁶ is C₃₋₇ cycloalkyl. In certain embodiments, R⁶ is cyclopropyl. In certain embodiments, R⁶ is C₃₋₇ cycloalkyl substituted with 1 occurrence of C₁₋₆ alkoxyl. In certain embodiments, R⁶ is C₃₋₇ cycloalkyl substituted with 1 occurrence of —OCH₃. In certain embodiments, R⁶ is halo. In certain embodiments, R⁶ represents independently for each occurrence C₁₋₆ haloalkyl, —C(O)R⁷, or —C(O)N(R⁸)(R⁹). In certain embodiments, R⁶ is hydroxyl. In certain embodiments, R⁶ is C₁₋₆ alkyl. In certain embodiments, R⁶ is C₁₋₆ alkoxyl. In certain embodiments, R⁶ is —O—C₃₋₇ cycloalkyl. In certain embodiments, R⁶ is —C(O)R⁷. In certain embodiments, R⁶ is —N(R⁸)(R⁹). In certain embodiments, R⁶ is —N(CH₃)₂. In certain embodiments, R⁶ is oxo. In certain embodiments, R⁶ is C₁₋₄ hydroxyalkyl. In certain embodiments, R⁶ is C₁ hydroxyalkyl. In certain embodiments, R⁶ is C₂ hydroxyalkyl. In certain embodiments, R⁶ is C₃ hydroxyalkyl. In certain embodiments, R⁶ is C₄ hydroxyalkyl. In certain embodiments, R⁶ is —C(O)N(R⁸)(R⁹). In certain embodiments, R⁶ is —N(R⁸)C(O)R¹⁰. In certain embodiments, R⁶ is selected from the groups depicted in the compounds in Table 1 below.

As defined generally above, R⁷ is —OH, C₁₋₄ alkyl, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is —OH. In certain embodiments, R⁷ is C₁₋₄ alkyl. In certain embodiments, R⁷ is C₁ alkyl. In certain embodiments, R⁷ is C₂ alkyl. In certain embodiments, R⁷ is C₃ alkyl. In certain embodiments, R⁷ is C₄ alkyl. In certain embodiments, R⁷ is —O—(C₁₋₆ alkyl). In certain embodiments, R⁷ is —O—C₃₋₇ cycloalkyl. In certain embodiments, R⁷ is a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 4-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 5-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 6-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is selected from the groups depicted in the compounds in Table 1 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 1 below. In certain embodiments, R⁹ is selected from the groups depicted in the compounds in Table 1 below.

As defined generally above, R¹⁰ represents independently for each occurrence C₁₋₆ alkyl or —(C₀₋₅ alkylene)-C₃₋₇ cycloalkyl. In certain embodiments, R¹⁰ is C₁₋₆ alkyl. In certain embodiments, R¹⁰ is —(C₀₋₅ alkylene)-C₃₋₇ cycloalkyl. In certain embodiments, R¹⁰ is selected from the groups depicted in the compounds in Table 1 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ is halo. In certain embodiments, R¹¹ is hydroxyl. In certain embodiments, R¹¹ is C₁₋₆ alkyl. In certain embodiments, R¹¹ is C₁₋₆ haloalkyl. In certain embodiments, R¹¹ is C₁₋₆ alkoxyl. In certain embodiments, R¹¹ is C₃₋₇ cycloalkyl. In certain embodiments, R¹¹ is selected from the groups depicted in the compounds in Table 1 below.

As defined generally above, A¹ is a 5-6 membered heteroaryl containing 1, 2, 3, or 4 heteroatoms independently selected from oxygen, nitrogen, and sulfur, wherein the heteroaryl is substituted with n occurrences of R⁶. In certain embodiments, A¹ is pyrazolyl, imidazolyl, oxazolyl, isoxazolyl, isothiazolyl, pyrrolyl, or furanyl, each of which is substituted with n occurrences of R⁶. In certain embodiments, A¹ is pyrazolyl substituted with n occurrences of R⁶. In certain embodiments, A¹ is

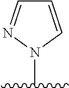 [SMILES: *n1cccn1]

substituted with n occurrences of R⁶. In certain embodiments, A¹ is

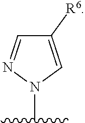

In certain embodiments, A¹ is a 5-membered heteroaryl containing 1 heteratom independently selected from oxygen, nitrogen, and sulfur, wherein the heteroaryl is substituted with n occurrences of R⁶. In certain embodiments, A¹ is a 5-membered heteroaryl containing 2 heteroatoms independently selected from oxygen, nitrogen, and sulfur, wherein the heteroaryl is substituted with n occurrences of R⁶. In certain embodiments, A¹ is a 5-membered heteroaryl containing 3 heteroatoms independently selected from oxygen, nitrogen, and sulfur, wherein the heteroaryl is substituted with n occurrences of R⁶. In certain embodiments, A¹ is a 5-membered heteroaryl containing 4 heteroatoms independently selected from oxygen, nitrogen, and sulfur, wherein the heteroaryl is substituted with n occurrences of R⁶.

In certain embodiments, A¹ is a 6-membered heteroaryl containing 1 heteratom independently selected from oxygen, nitrogen, and sulfur, wherein the heteroaryl is substituted with n occurrences of R⁶. In certain embodiments, A¹ is a 6-membered heteroaryl containing 2 heteroatoms independently selected from oxygen, nitrogen, and sulfur, wherein the heteroaryl is substituted with n occurrences of R⁶. In certain embodiments, A¹ is a 6-membered heteroaryl containing 3 heteroatoms independently selected from oxygen, nitrogen, and sulfur, wherein the heteroaryl is substituted with n occurrences of R⁶. In certain embodiments, A¹ is a 6-membered heteroaryl containing 4 heteroatoms independently selected from oxygen, nitrogen, and sulfur, wherein the heteroaryl is substituted with n occurrences of R⁶. In certain embodiments, A¹ is selected from the groups depicted in the compounds in Table 1 below.

As defined generally above, y is 1 or 2. In certain embodiments, y is 1. In certain embodiments, y is 2. In certain embodiments, y is selected from the corresponding value in the groups depicted in the compounds in Table 1 below.

As defined generally above, m and x are independently 0, 1, or 2. In certain embodiments, x is 0. In certain embodiments, m is 0. In certain embodiments, m is 1. In certain embodiments, m is 2. In certain embodiments, x is 1. In certain embodiments, x is 2. In certain embodiments, m is selected from the corresponding value in the groups depicted in the compounds in Table 1 below. In certain embodiments, x is selected from the corresponding value in the groups depicted in the compounds in Table 1 below.

As defined generally above, n is 0, 1, 2, or 3. In certain embodiments, n is 0. In certain embodiments, n is 1. In certain embodiments, n is 2. In certain embodiments, n is 3. In certain embodiments, n is selected from the corresponding value in the groups depicted in the compounds in Table 1 below.

The description above describes multiple embodiments relating to compounds of Formula I-C. The patent application specifically contemplates all combinations of the embodiments.

### Part F:

Another aspect of the invention provides a compound in Table 1 below, or a pharmaceutically acceptable salt thereof. In certain embodiments, the compound is a compound in Table 1 below. In certain embodiments, the compound is any one of compounds I-1 to I-105 in Table 1 below, or a pharmaceutically acceptable salt thereof. In certain embodiments, the compound is any one of compounds I-1 to I-105 in Table 1 below.

TABLE 1

| Compound No. | Structure |
|---|---|
| I-1 | 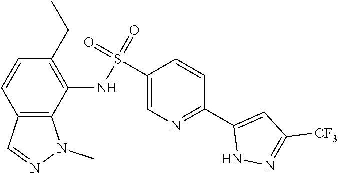 [SMILES: CCc1ccc2cnn(C)c2c1NS(=O)(=O)c1ccc(-c2cc(C(F)(F)F)n[nH]2)nc1] |
| I-2 | 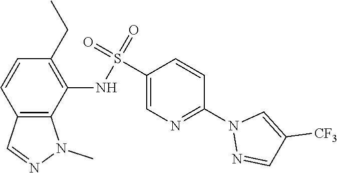 [SMILES: CCc1ccc2cnn(C)c2c1NS(=O)(=O)c1ccc(-n2cc(C(F)(F)F)cn2)nc1] |
| I-3 | 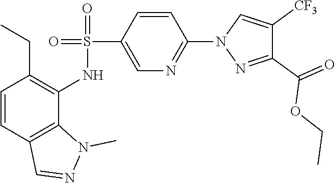 [SMILES: CCOC(=O)c1nn(-c2ccc(S(=O)(=O)Nc3c(CC)ccc4cnn(C)c34)cn2)cc1C(F)(F)F] |
| I-4 | 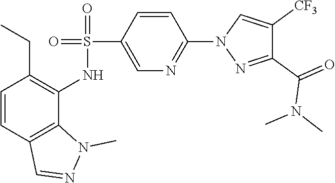 [SMILES: CCc1ccc2cnn(C)c2c1NS(=O)(=O)c1ccc(-n2cc(C(F)(F)F)c(C(=O)N(C)C)n2)nc1] |
| I-5 | 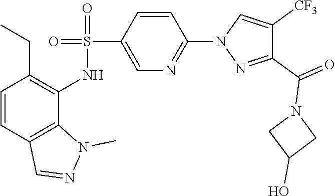 [SMILES: CCc1ccc2cnn(C)c2c1NS(=O)(=O)c1ccc(-n2cc(C(F)(F)F)c(C(=O)N3CC(O)C3)n2)nc1] |
| I-6 | 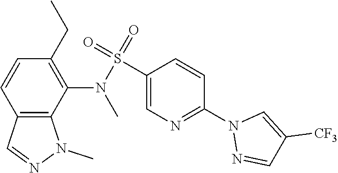 [SMILES: CCc1ccc2cnn(C)c2c1N(C)S(=O)(=O)c1ccc(-n2cc(C(F)(F)F)cn2)nc1] |
| I-7 | 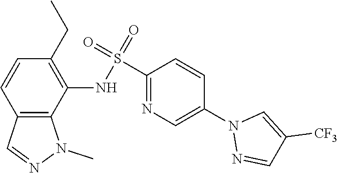 [SMILES: CCc1ccc2cnn(C)c2c1NS(=O)(=O)c1ccc(-n2cc(C(F)(F)F)cn2)cn1] |
| I-8 | 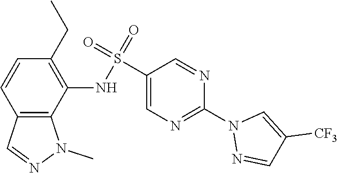 [SMILES: CCc1ccc2cnn(C)c2c1NS(=O)(=O)c1cnc(-n2cc(C(F)(F)F)cn2)nc1] |
| I-9 | 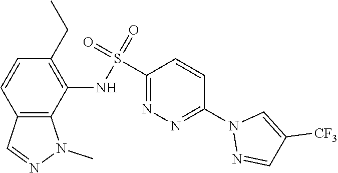 [SMILES: CCc1ccc2cnn(C)c2c1NS(=O)(=O)c1ccc(-n2cc(C(F)(F)F)cn2)nn1] |
| I-10 | 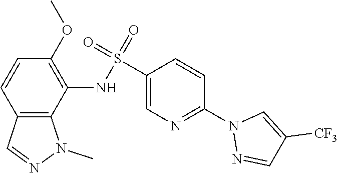 [SMILES: COc1ccc2cnn(C)c2c1NS(=O)(=O)c1ccc(-n2cc(C(F)(F)F)cn2)nc1] |
| I-11 | 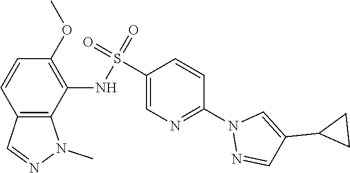 [SMILES: COc1ccc2cnn(C)c2c1NS(=O)(=O)c1ccc(-n2cc(C3CC3)cn2)nc1] |
| I-12 | 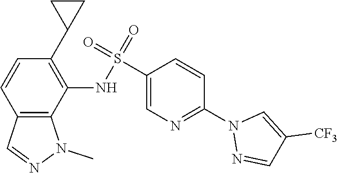 [SMILES: Cn1ncc2ccc(C3CC3)c(NS(=O)(=O)c3ccc(-n4cc(C(F)(F)F)cn4)nc3)c21] |
| I-13 | 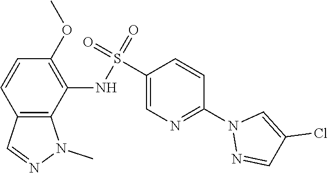 [SMILES: COc1ccc2cnn(C)c2c1NS(=O)(=O)c1ccc(-n2cc(Cl)cn2)nc1] |
| I-14 | 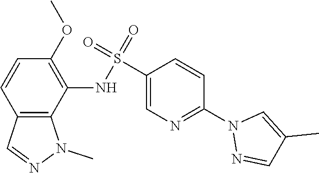 [SMILES: COc1ccc2cnn(C)c2c1NS(=O)(=O)c1ccc(-n2cc(C)cn2)nc1] |
| I-15 | 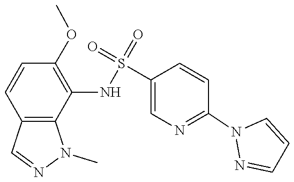 [SMILES: COc1ccc2cnn(C)c2c1NS(=O)(=O)c1ccc(-n2cccn2)nc1] |
| I-16 | 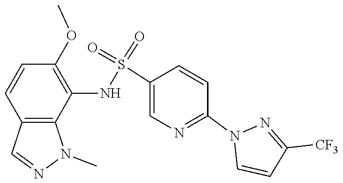 [SMILES: COc1ccc2cnn(C)c2c1NS(=O)(=O)c1ccc(-n2ccc(C(F)(F)F)n2)nc1] |
| I-17 | 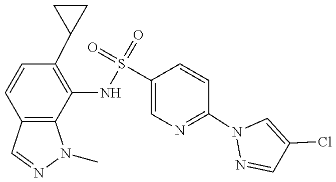 [SMILES: Cn1ncc2ccc(C3CC3)c(NS(=O)(=O)c3ccc(-n4cc(Cl)cn4)nc3)c21] |
| I-18 | 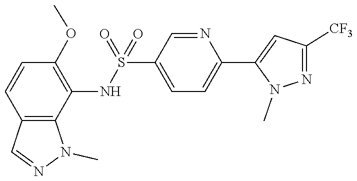 [SMILES: COc1ccc2cnn(C)c2c1NS(=O)(=O)c1ccc(-c2cc(C(F)(F)F)nn2C)nc1] |
| I-19 | 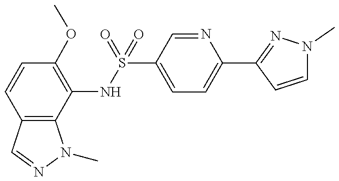 [SMILES: COc1ccc2cnn(C)c2c1NS(=O)(=O)c1ccc(-c2ccn(C)n2)nc1] |
| I-20 | 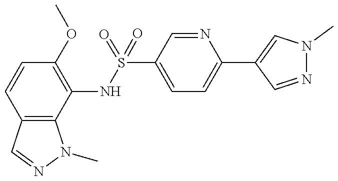 [SMILES: COc1ccc2cnn(C)c2c1NS(=O)(=O)c1ccc(-c2cnn(C)c2)nc1] |
| I-21 | 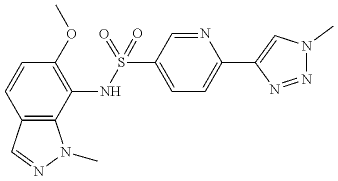 [SMILES: COc1ccc2cnn(C)c2c1NS(=O)(=O)c1ccc(-c2cn(C)nn2)nc1] |
| I-22 | 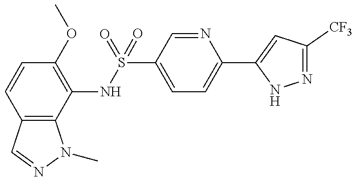 [SMILES: COc1ccc2cnn(C)c2c1NS(=O)(=O)c1ccc(-c2cc(C(F)(F)F)n[nH]2)nc1] |
| I-23 | 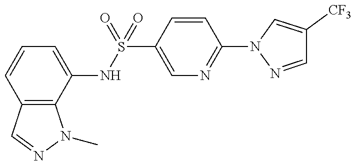 [SMILES: Cn1ncc2cccc(NS(=O)(=O)c3ccc(-n4cc(C(F)(F)F)cn4)nc3)c21] |
| I-24 | 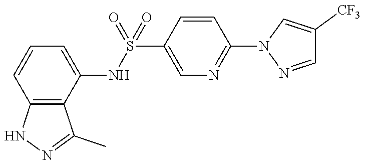 [SMILES: Cc1n[nH]c2cccc(NS(=O)(=O)c3ccc(-n4cc(C(F)(F)F)cn4)nc3)c12] |
| I-25 | 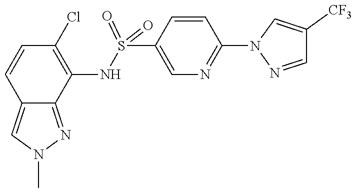 [SMILES: Cn1cc2ccc(Cl)c(NS(=O)(=O)c3ccc(-n4cc(C(F)(F)F)cn4)nc3)c2n1] |
| I-26 | 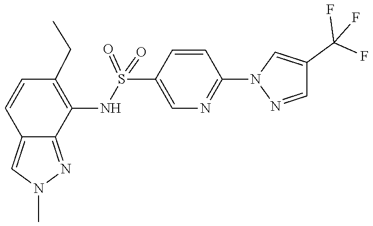 [SMILES: CCc1ccc2cn(C)nc2c1NS(=O)(=O)c1ccc(-n2cc(C(F)(F)F)cn2)nc1] |
| I-27 | 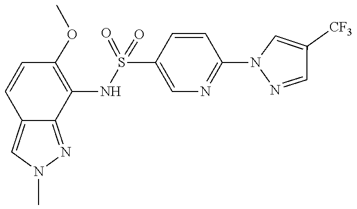 [SMILES: COc1ccc2cn(C)nc2c1NS(=O)(=O)c1ccc(-n2cc(C(F)(F)F)cn2)nc1] |
| I-28 | 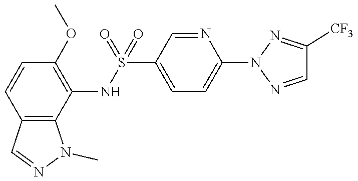 [SMILES: COc1ccc2cnn(C)c2c1NS(=O)(=O)c1ccc(-n2ncc(C(F)(F)F)n2)nc1] |
| I-29 | 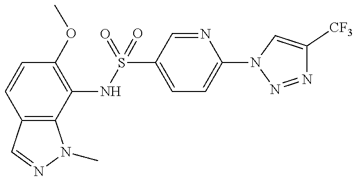 [SMILES: COc1ccc2cnn(C)c2c1NS(=O)(=O)c1ccc(-n2cc(C(F)(F)F)nn2)nc1] |
| I-30 | 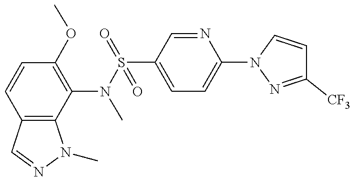 [SMILES: COc1ccc2cnn(C)c2c1N(C)S(=O)(=O)c1ccc(-n2ccc(C(F)(F)F)n2)nc1] |
| I-31 | 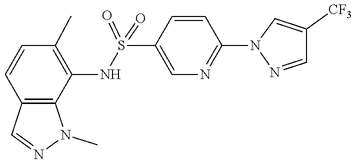 [SMILES: Cc1ccc2cnn(C)c2c1NS(=O)(=O)c1ccc(-n2cc(C(F)(F)F)cn2)nc1] |
| I-32 | 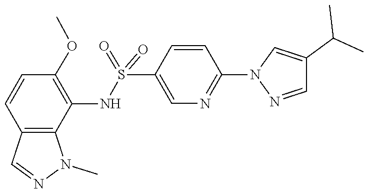 [SMILES: COc1ccc2cnn(C)c2c1NS(=O)(=O)c1ccc(-n2cc(C(C)C)cn2)nc1] |
| I-33 | 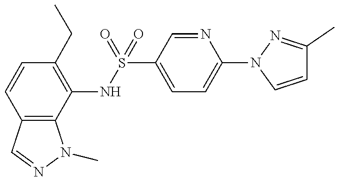 [SMILES: CCc1ccc2cnn(C)c2c1NS(=O)(=O)c1ccc(-n2ccc(C)n2)nc1] |
| I-34 | 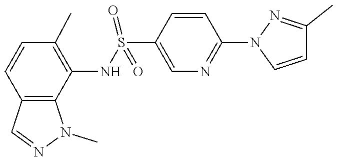 [SMILES: Cc1ccn(-c2ccc(S(=O)(=O)Nc3c(C)ccc4cnn(C)c34)cn2)n1] |
| I-35 | 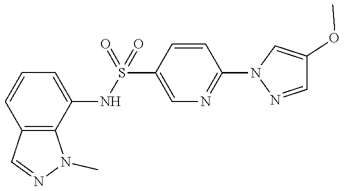 [SMILES: COc1cnn(-c2ccc(S(=O)(=O)Nc3cccc4cnn(C)c34)cn2)c1] |
| I-36 | 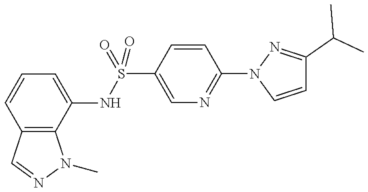 [SMILES: CC(C)c1ccn(-c2ccc(S(=O)(=O)Nc3cccc4cnn(C)c34)cn2)n1] |
| I-37 | 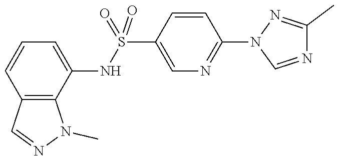 [SMILES: Cc1ncn(-c2ccc(S(=O)(=O)Nc3cccc4cnn(C)c34)cn2)n1] |
| I-38 | 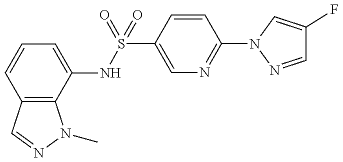 [SMILES: Cn1ncc2cccc(NS(=O)(=O)c3ccc(-n4cc(F)cn4)nc3)c21] |
| I-39 | 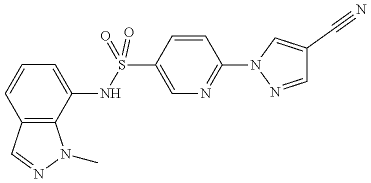 [SMILES: Cn1ncc2cccc(NS(=O)(=O)c3ccc(-n4cc(C#N)cn4)nc3)c21] |
| I-40 | 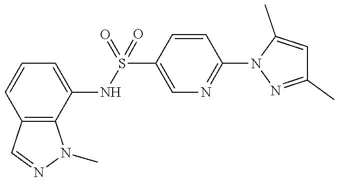 [SMILES: Cc1cc(C)n(-c2ccc(S(=O)(=O)Nc3cccc4cnn(C)c34)cn2)n1] |
| I-41 | 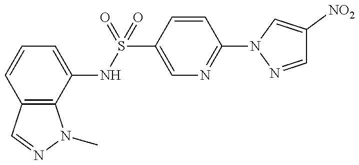 [SMILES: Cn1ncc2cccc(NS(=O)(=O)c3ccc(-n4cc([N+](=O)[O-])cn4)nc3)c21] |
| I-42 | 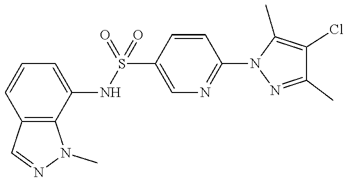 [SMILES: Cc1nn(-c2ccc(S(=O)(=O)Nc3cccc4cnn(C)c34)cn2)c(C)c1Cl] |
| I-43 | 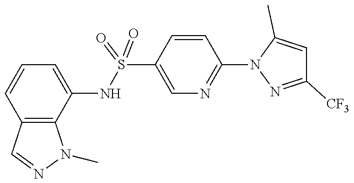 [SMILES: Cc1cc(C(F)(F)F)nn1-c1ccc(S(=O)(=O)Nc2cccc3cnn(C)c23)cn1] |
| I-44 | 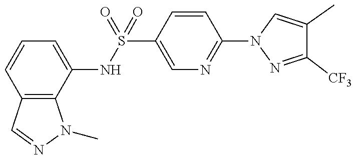 [SMILES: Cc1cn(-c2ccc(S(=O)(=O)Nc3cccc4cnn(C)c34)cn2)nc1C(F)(F)F] |
| I-45 | 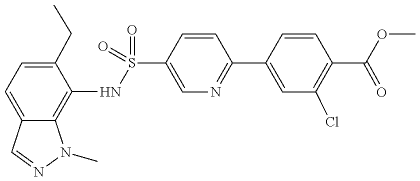 [SMILES: CCc1ccc2cnn(C)c2c1NS(=O)(=O)c1ccc(-c2ccc(C(=O)OC)c(Cl)c2)nc1] |
| I-46 | 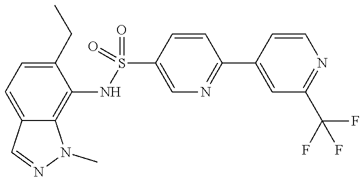 [SMILES: CCc1ccc2cnn(C)c2c1NS(=O)(=O)c1ccc(-c2ccnc(C(F)(F)F)c2)nc1] |
| I-47 | 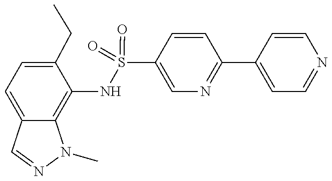 [SMILES: CCc1ccc2cnn(C)c2c1NS(=O)(=O)c1ccc(-c2ccncc2)nc1] |
| I-48 | 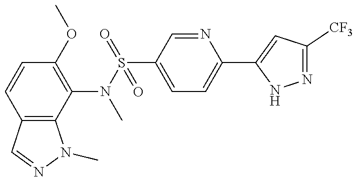 [SMILES: COc1ccc2cnn(C)c2c1N(C)S(=O)(=O)c1ccc(-c2cc(C(F)(F)F)n[nH]2)nc1] |
| I-49 | 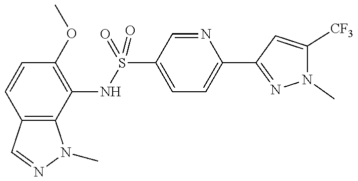 [SMILES: COc1ccc2cnn(C)c2c1NS(=O)(=O)c1ccc(-c2cc(C(F)(F)F)n(C)n2)nc1] |
| I-50 | 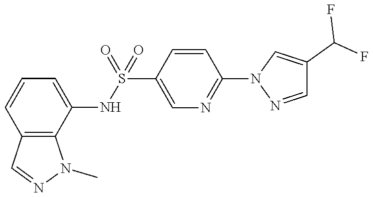 [SMILES: Cn1ncc2cccc(NS(=O)(=O)c3ccc(-n4cc(C(F)F)cn4)nc3)c21] |
| I-51 | 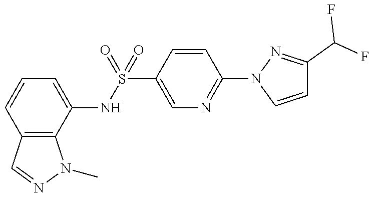 [SMILES: Cn1ncc2cccc(NS(=O)(=O)c3ccc(-n4ccc(C(F)F)n4)nc3)c21] |
| I-52 | 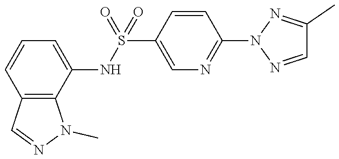 [SMILES: Cc1cnn(-c2ccc(S(=O)(=O)Nc3cccc4cnn(C)c34)cn2)n1] |
| I-53 | 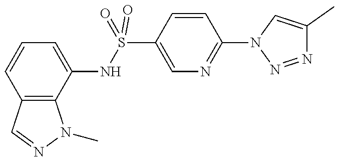 [SMILES: Cc1cn(-c2ccc(S(=O)(=O)Nc3cccc4cnn(C)c34)cn2)nn1] |
| I-54 | 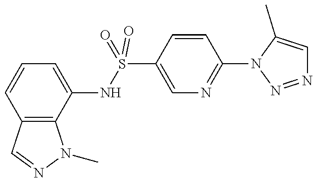 [SMILES: Cc1cnnn1-c1ccc(S(=O)(=O)Nc2cccc3cnn(C)c23)cn1] |
| I-55 | 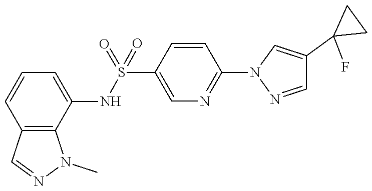 [SMILES: Cn1ncc2cccc(NS(=O)(=O)c3ccc(-n4cc(C5(F)CC5)cn4)nc3)c21] |
| I-56 | 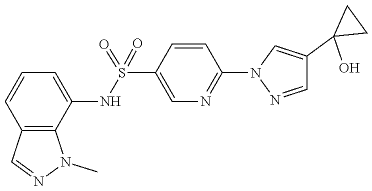 [SMILES: Cn1ncc2cccc(NS(=O)(=O)c3ccc(-n4cc(C5(O)CC5)cn4)nc3)c21] |
| I-57 | 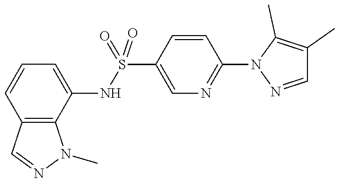 [SMILES: Cc1cnn(-c2ccc(S(=O)(=O)Nc3cccc4cnn(C)c34)cn2)c1C] |
| I-58 | 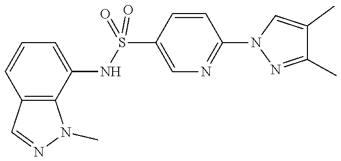 [SMILES: Cc1cn(-c2ccc(S(=O)(=O)Nc3cccc4cnn(C)c34)cn2)nc1C] |
| I-59 | 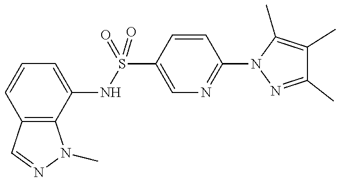 [SMILES: Cc1nn(-c2ccc(S(=O)(=O)Nc3cccc4cnn(C)c34)cn2)c(C)c1C] |
| I-60 | 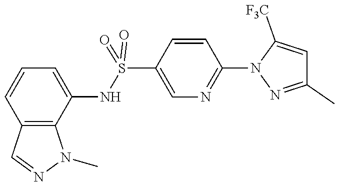 [SMILES: Cc1cc(C(F)(F)F)n(-c2ccc(S(=O)(=O)Nc3cccc4cnn(C)c34)cn2)n1] |
| I-61 | 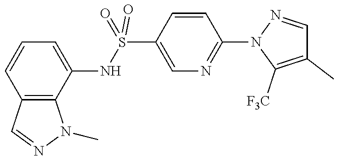 [SMILES: Cc1cnn(-c2ccc(S(=O)(=O)Nc3cccc4cnn(C)c34)cn2)c1C(F)(F)F] |
| I-62 | 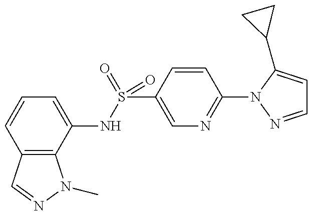 [SMILES: Cn1ncc2cccc(NS(=O)(=O)c3ccc(-n4nccc4C4CC4)nc3)c21] |
| I-63 | 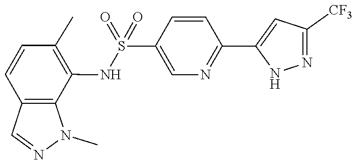 [SMILES: Cc1ccc2cnn(C)c2c1NS(=O)(=O)c1ccc(-c2cc(C(F)(F)F)n[nH]2)nc1] |
| I-64 | 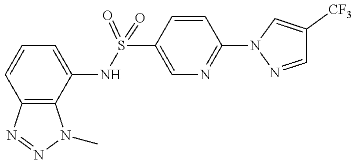 [SMILES: Cn1nnc2cccc(NS(=O)(=O)c3ccc(-n4cc(C(F)(F)F)cn4)nc3)c21] |
| I-65 | 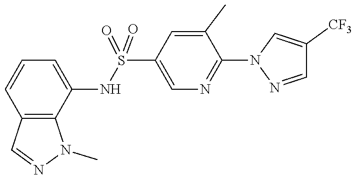 [SMILES: Cc1cc(S(=O)(=O)Nc2cccc3cnn(C)c23)cnc1-n1cc(C(F)(F)F)cn1] |
| I-66 | 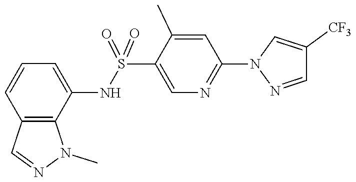 [SMILES: Cc1cc(-n2cc(C(F)(F)F)cn2)ncc1S(=O)(=O)Nc1cccc2cnn(C)c12] |
| I-67 | 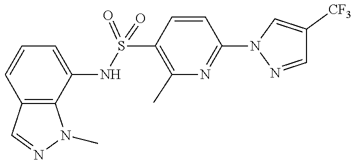 [SMILES: Cc1nc(-n2cc(C(F)(F)F)cn2)ccc1S(=O)(=O)Nc1cccc2cnn(C)c12] |
| I-68 | 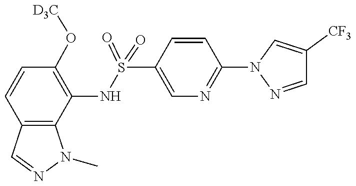 [SMILES: [2H]C([2H])([2H])Oc1ccc2cnn(C)c2c1NS(=O)(=O)c1ccc(-n2cc(C(F)(F)F)cn2)nc1] |
| I-69 | 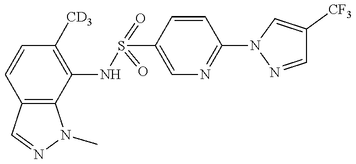 [SMILES: [2H]C([2H])([2H])c1ccc2cnn(C)c2c1NS(=O)(=O)c1ccc(-n2cc(C(F)(F)F)cn2)nc1] |
| I-70 | 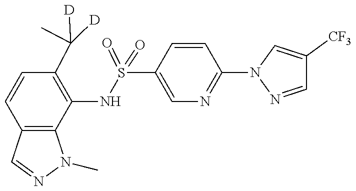 [SMILES: [2H]C([2H])(C)c1ccc2cnn(C)c2c1NS(=O)(=O)c1ccc(-n2cc(C(F)(F)F)cn2)nc1] |
| I-71 | 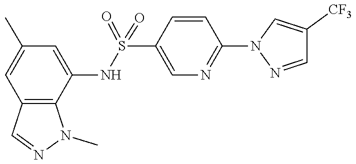 [SMILES: Cc1cc(NS(=O)(=O)c2ccc(-n3cc(C(F)(F)F)cn3)nc2)c2c(cnn2C)c1] |
| I-72 | 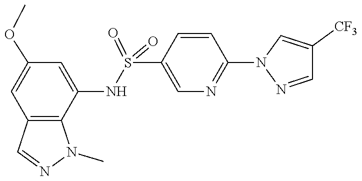 [SMILES: COc1cc(NS(=O)(=O)c2ccc(-n3cc(C(F)(F)F)cn3)nc2)c2c(cnn2C)c1] |
| I-73 | 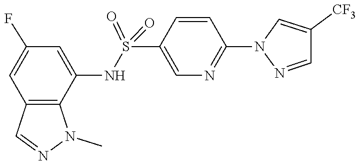 [SMILES: Cn1ncc2cc(F)cc(NS(=O)(=O)c3ccc(-n4cc(C(F)(F)F)cn4)nc3)c21] |
| I-74 | 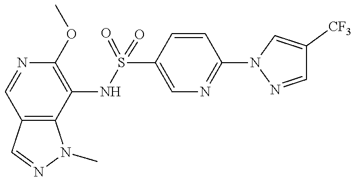 [SMILES: COc1ncc2cnn(C)c2c1NS(=O)(=O)c1ccc(-n2cc(C(F)(F)F)cn2)nc1] |
| I-75 | 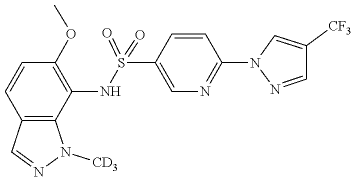 [SMILES: [2H]C([2H])([2H])n1ncc2ccc(OC)c(NS(=O)(=O)c3ccc(-n4cc(C(F)(F)F)cn4)nc3)c21] |
| I-76 | 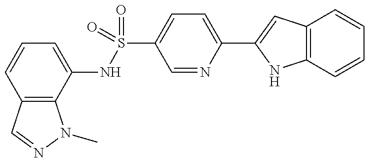 [SMILES: Cn1ncc2cccc(NS(=O)(=O)c3ccc(-c4cc5ccccc5[nH]4)nc3)c21] |
| I-77 | 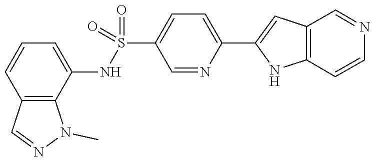 [SMILES: Cn1ncc2cccc(NS(=O)(=O)c3ccc(-c4cc5cnccc5[nH]4)nc3)c21] |
| I-78 | 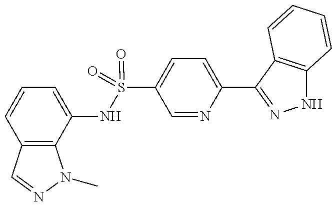 [SMILES: Cn1ncc2cccc(NS(=O)(=O)c3ccc(-c4n[nH]c5ccccc45)nc3)c21] |
| I-79 | 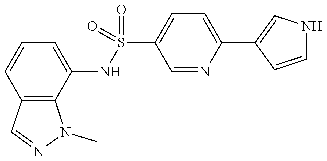 [SMILES: Cn1ncc2cccc(NS(=O)(=O)c3ccc(-c4cc[nH]c4)nc3)c21] |
| I-80 | 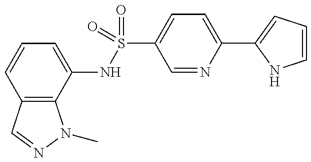 [SMILES: Cn1ncc2cccc(NS(=O)(=O)c3ccc(-c4ccc[nH]4)nc3)c21] |
| I-81 | 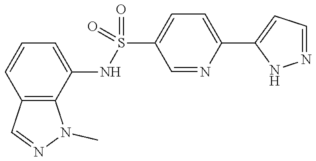 [SMILES: Cn1ncc2cccc(NS(=O)(=O)c3ccc(-c4ccn[nH]4)nc3)c21] |
| I-82 | 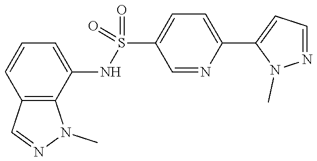 [SMILES: Cn1nccc1-c1ccc(S(=O)(=O)Nc2cccc3cnn(C)c23)cn1] |
| I-83 | 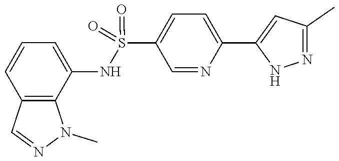 [SMILES: Cc1cc(-c2ccc(S(=O)(=O)Nc3cccc4cnn(C)c34)cn2)[nH]n1] |
| I-84 | 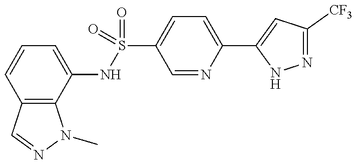 [SMILES: Cn1ncc2cccc(NS(=O)(=O)c3ccc(-c4cc(C(F)(F)F)n[nH]4)nc3)c21] |
| I-85 | 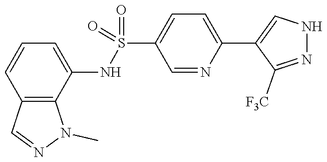 [SMILES: Cn1ncc2cccc(NS(=O)(=O)c3ccc(-c4c[nH]nc4C(F)(F)F)nc3)c21] |
| I-86 | 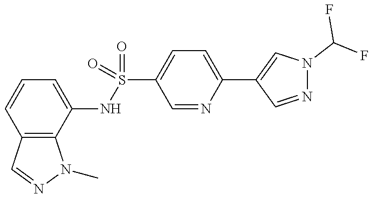 [SMILES: Cn1ncc2cccc(NS(=O)(=O)c3ccc(-c4cnn(C(F)F)c4)nc3)c21] |
| I-87 | 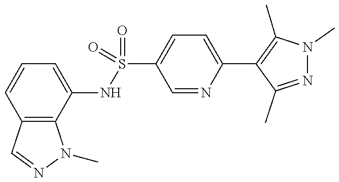 [SMILES: Cc1nn(C)c(C)c1-c1ccc(S(=O)(=O)Nc2cccc3cnn(C)c23)cn1] |
| I-88 | 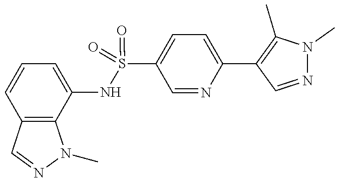 [SMILES: Cc1c(-c2ccc(S(=O)(=O)Nc3cccc4cnn(C)c34)cn2)cnn1C] |
| I-89 | 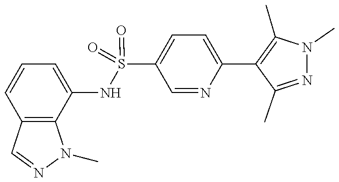 [SMILES: Cc1nn(C)c(C)c1-c1ccc(S(=O)(=O)Nc2cccc3cnn(C)c23)cn1] |
| I-90 | 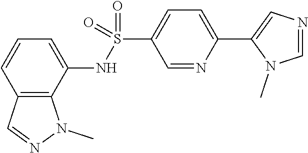 [SMILES: Cn1cncc1-c1ccc(S(=O)(=O)Nc2cccc3cnn(C)c23)cn1] |
| I-91 | 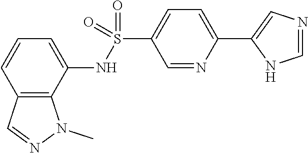 [SMILES: Cn1ncc2cccc(NS(=O)(=O)c3ccc(-c4cnc[nH]4)nc3)c21] |
| I-92 | 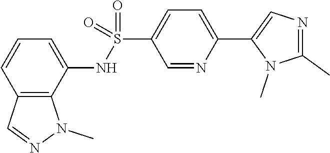 [SMILES: Cc1ncc(-c2ccc(S(=O)(=O)Nc3cccc4cnn(C)c34)cn2)n1C] |
| I-93 | 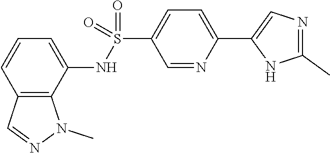 [SMILES: Cc1ncc(-c2ccc(S(=O)(=O)Nc3cccc4cnn(C)c34)cn2)[nH]1] |
| I-94 | 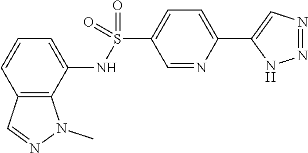 [SMILES: Cn1ncc2cccc(NS(=O)(=O)c3ccc(-c4cnn[nH]4)nc3)c21] |
| I-95 | 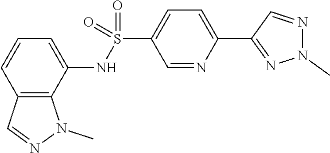 [SMILES: Cn1ncc(-c2ccc(S(=O)(=O)Nc3cccc4cnn(C)c34)cn2)n1] |
| I-96 | 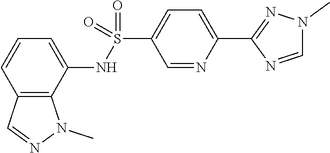 [SMILES: Cn1cnc(-c2ccc(S(=O)(=O)Nc3cccc4cnn(C)c34)cn2)n1] |
| I-97 | 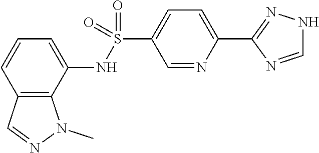 [SMILES: Cn1ncc2cccc(NS(=O)(=O)c3ccc(-c4nc[nH]n4)nc3)c21] |
| I-98 | 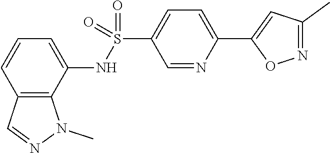 [SMILES: Cc1cc(-c2ccc(S(=O)(=O)Nc3cccc4cnn(C)c34)cn2)on1] |
| I-99 | 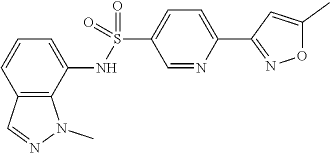 [SMILES: Cc1cc(-c2ccc(S(=O)(=O)Nc3cccc4cnn(C)c34)cn2)no1] |
| I-100 | 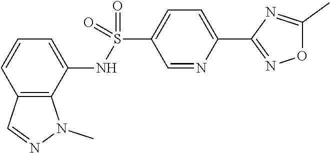 [SMILES: Cc1nc(-c2ccc(S(=O)(=O)Nc3cccc4cnn(C)c34)cn2)no1] |
| I-101 | 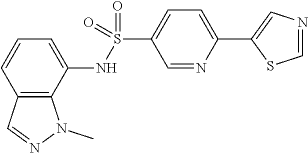 [SMILES: Cn1ncc2cccc(NS(=O)(=O)c3ccc(-c4cncs4)nc3)c21] |
| I-102 | 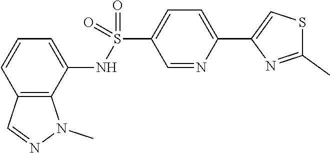 [SMILES: Cc1nc(-c2ccc(S(=O)(=O)Nc3cccc4cnn(C)c34)cn2)cs1] |
| I-103 | 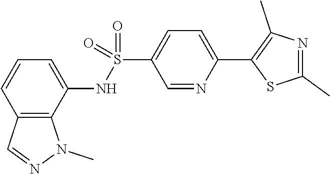 [SMILES: Cc1nc(C)c(-c2ccc(S(=O)(=O)Nc3cccc4cnn(C)c34)cn2)s1] |
| I-104 | 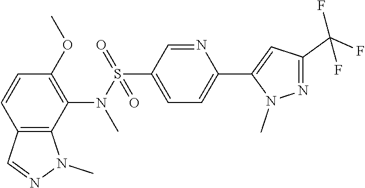 [SMILES: COc1ccc2cnn(C)c2c1N(C)S(=O)(=O)c1ccc(-c2cc(C(F)(F)F)nn2C)nc1] |
| I-105 | 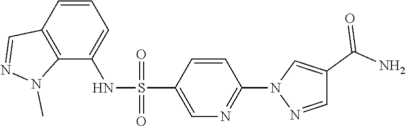 [SMILES: Cn1ncc2cccc(NS(=O)(=O)c3ccc(-n4cc(C(N)=O)cn4)nc3)c21] |
| I-106 | 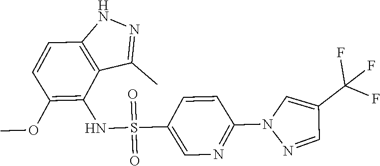 [SMILES: COc1ccc2[nH]nc(C)c2c1NS(=O)(=O)c1ccc(-n2cc(C(F)(F)F)cn2)nc1] |
| I-107 | 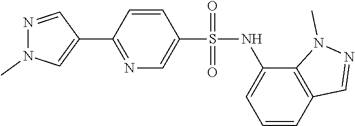 [SMILES: Cn1cc(-c2ccc(S(=O)(=O)Nc3cccc4cnn(C)c34)cn2)cn1] |
| I-108 | 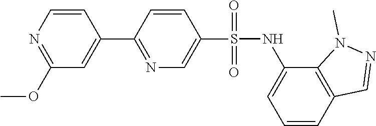 [SMILES: COc1cc(-c2ccc(S(=O)(=O)Nc3cccc4cnn(C)c34)cn2)ccn1] |
| I-109 | 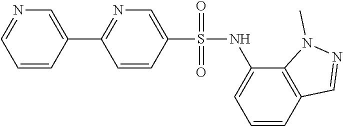 [SMILES: Cn1ncc2cccc(NS(=O)(=O)c3ccc(-c4cccnc4)nc3)c21] |
| I-110 | 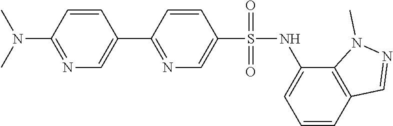 [SMILES: CN(C)c1ccc(-c2ccc(S(=O)(=O)Nc3cccc4cnn(C)c34)cn2)cn1] |
| I-111 | 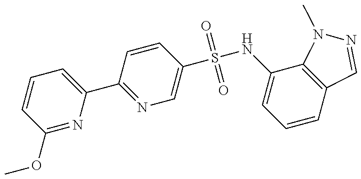 [SMILES: COc1cccc(-c2ccc(S(=O)(=O)Nc3cccc4cnn(C)c34)cn2)n1] |
| I-112 | 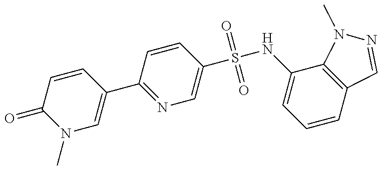 [SMILES: Cn1cc(-c2ccc(S(=O)(=O)Nc3cccc4cnn(C)c34)cn2)ccc1=O] |
| I-113 | 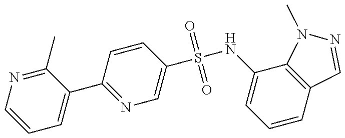 [SMILES: Cc1ncccc1-c1ccc(S(=O)(=O)Nc2cccc3cnn(C)c23)cn1] |
| I-114 | 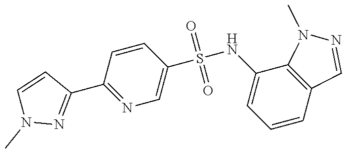 [SMILES: Cn1ccc(-c2ccc(S(=O)(=O)Nc3cccc4cnn(C)c34)cn2)n1] |
| I-115 | 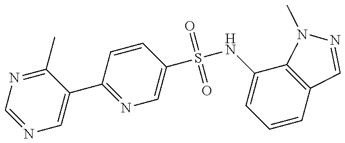 [SMILES: Cc1ncncc1-c1ccc(S(=O)(=O)Nc2cccc3cnn(C)c23)cn1] |
| I-116 | 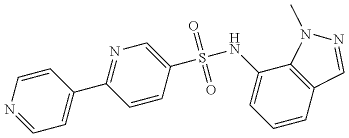 [SMILES: Cn1ncc2cccc(NS(=O)(=O)c3ccc(-c4ccncc4)nc3)c21] |
| I-117 | 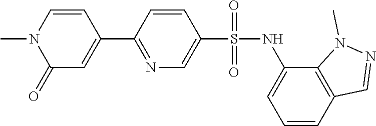 [SMILES: Cn1ccc(-c2ccc(S(=O)(=O)Nc3cccc4cnn(C)c34)cn2)cc1=O] |
| I-118 | 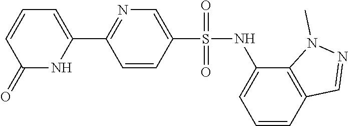 [SMILES: Cn1ncc2cccc(NS(=O)(=O)c3ccc(-c4cccc(=O)[nH]4)nc3)c21] |
| I-119 | 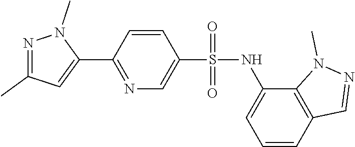 [SMILES: Cc1cc(-c2ccc(S(=O)(=O)Nc3cccc4cnn(C)c34)cn2)n(C)n1] |
| I-120 | 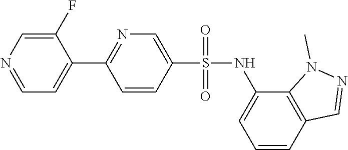 [SMILES: Cn1ncc2cccc(NS(=O)(=O)c3ccc(-c4ccncc4F)nc3)c21] |
| I-121 | 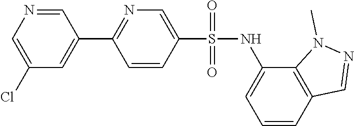 [SMILES: Cn1ncc2cccc(NS(=O)(=O)c3ccc(-c4cncc(Cl)c4)nc3)c21] |
| I-122 | 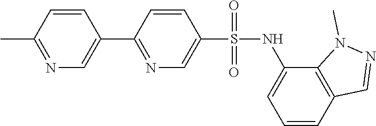 [SMILES: Cc1ccc(-c2ccc(S(=O)(=O)Nc3cccc4cnn(C)c34)cn2)cn1] |
| I-123 | 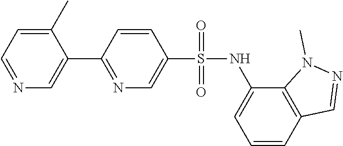 [SMILES: Cc1ccncc1-c1ccc(S(=O)(=O)Nc2cccc3cnn(C)c23)cn1] |
| I-124 | 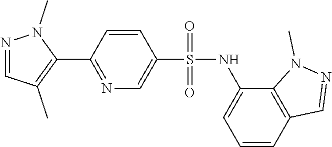 [SMILES: Cc1cnn(C)c1-c1ccc(S(=O)(=O)Nc2cccc3cnn(C)c23)cn1] |
| I-125 | 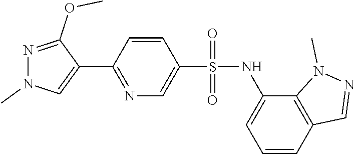 [SMILES: COc1nn(C)cc1-c1ccc(S(=O)(=O)Nc2cccc3cnn(C)c23)cn1] |
| I-126 | 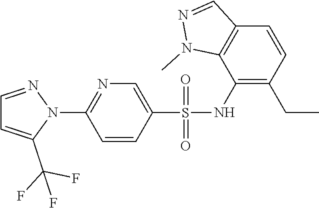 [SMILES: CCc1ccc2cnn(C)c2c1NS(=O)(=O)c1ccc(-n2nccc2C(F)(F)F)nc1] |
| I-127 | 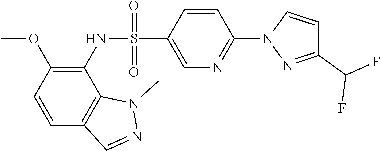 [SMILES: COc1ccc2cnn(C)c2c1NS(=O)(=O)c1ccc(-n2ccc(C(F)F)n2)nc1] |
| I-128 | 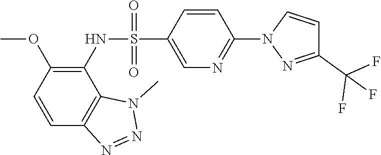 [SMILES: COc1ccc2nnn(C)c2c1NS(=O)(=O)c1ccc(-n2ccc(C(F)(F)F)n2)nc1] |
| I-129 | 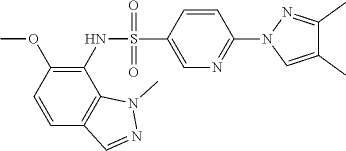 |
| I-130 | 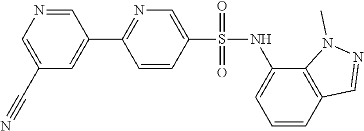 [SMILES: Cn1ncc2cccc(NS(=O)(=O)c3ccc(-c4cncc(C#N)c4)nc3)c21] |
| I-131 | 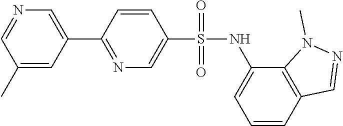 [SMILES: Cc1cncc(-c2ccc(S(=O)(=O)Nc3cccc4cnn(C)c34)cn2)c1] |
| I-132 | 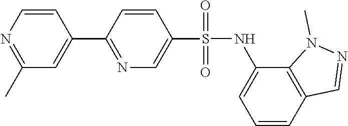 [SMILES: Cc1cc(-c2ccc(S(=O)(=O)Nc3cccc4cnn(C)c34)cn2)ccn1] |
| I-133 | 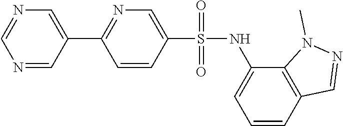 [SMILES: Cn1ncc2cccc(NS(=O)(=O)c3ccc(-c4cncnc4)nc3)c21] |
| I-134 | 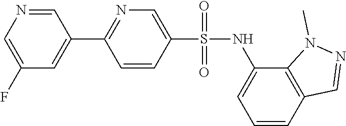 [SMILES: Cn1ncc2cccc(NS(=O)(=O)c3ccc(-c4cncc(F)c4)nc3)c21] |
| I-135 | 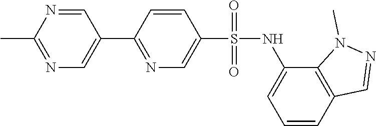 [SMILES: Cc1ncc(-c2ccc(S(=O)(=O)Nc3cccc4cnn(C)c34)cn2)cn1] |
| I-136 | 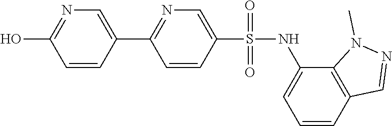 [SMILES: Cn1ncc2cccc(NS(=O)(=O)c3ccc(-c4ccc(O)nc4)nc3)c21] |
| I-137 | 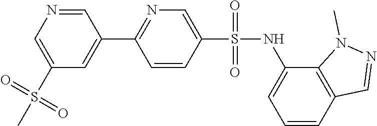 [SMILES: Cn1ncc2cccc(NS(=O)(=O)c3ccc(-c4cncc(S(C)(=O)=O)c4)nc3)c21] |
| I-138 | 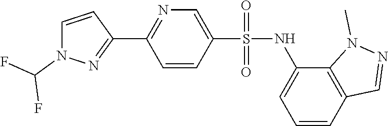 [SMILES: Cn1ncc2cccc(NS(=O)(=O)c3ccc(-c4ccn(C(F)F)n4)nc3)c21] |
| I-139 | 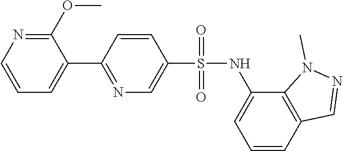 [SMILES: COc1ncccc1-c1ccc(S(=O)(=O)Nc2cccc3cnn(C)c23)cn1] |
| I-140 | 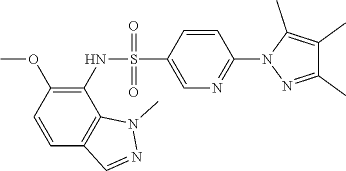 |
| I-141 | 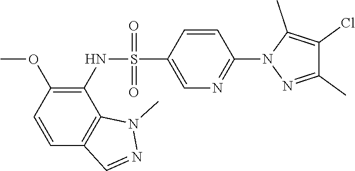 [SMILES: COc1ccc2cnn(C)c2c1NS(=O)(=O)c1ccc(-n2nc(C)c(Cl)c2C)nc1] |
| I-142 | 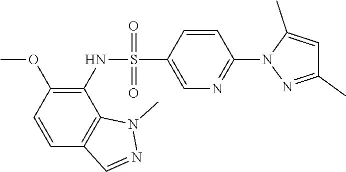 |
| I-143 | 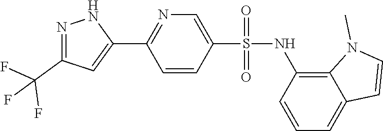 [SMILES: Cn1ccc2cccc(NS(=O)(=O)c3ccc(-c4cc(C(F)(F)F)n[nH]4)nc3)c21] |
| I-144 | 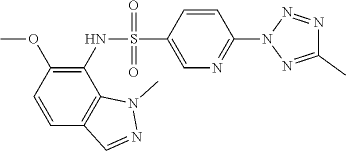 |
| I-145 | 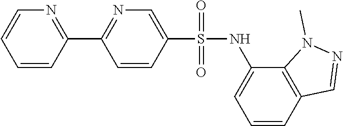 [SMILES: Cn1ncc2cccc(NS(=O)(=O)c3ccc(-c4ccccn4)nc3)c21] |
| I-146 | 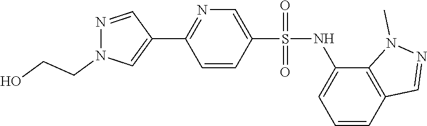 [SMILES: Cn1ncc2cccc(NS(=O)(=O)c3ccc(-c4cnn(CCO)c4)nc3)c21] |
| I-147 | 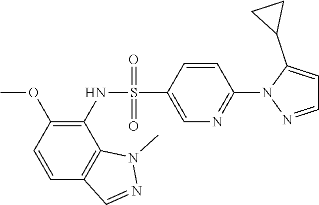 [SMILES: COc1ccc2cnn(C)c2c1NS(=O)(=O)c1ccc(-n2nccc2C2CC2)nc1] |
| I-148 | 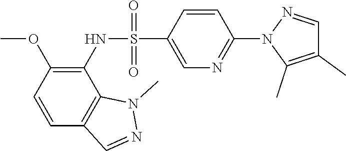 |
| I-149 | 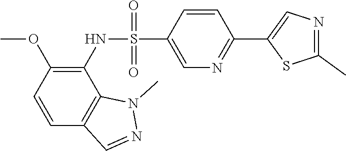 |
| I-150 | 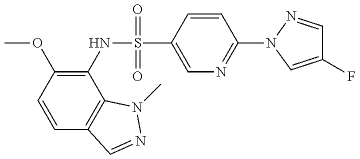 [SMILES: COc1ccc2cnn(C)c2c1NS(=O)(=O)c1ccc(-n2cc(F)cn2)nc1] |
| I-151 | 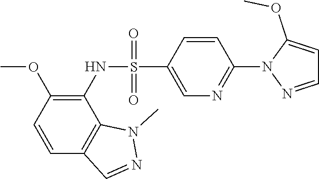 [SMILES: COc1ccc2cnn(C)c2c1NS(=O)(=O)c1ccc(-n2nccc2OC)nc1] |
| I-152 | 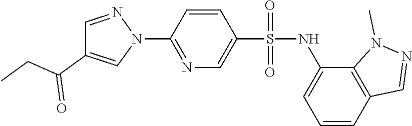 [SMILES: CCC(=O)c1cnn(-c2ccc(S(=O)(=O)Nc3cccc4cnn(C)c34)cn2)c1] |
| I-153 | 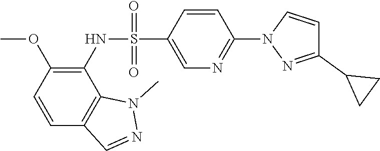 [SMILES: COc1ccc2cnn(C)c2c1NS(=O)(=O)c1ccc(-n2ccc(C3CC3)n2)nc1] |
| I-154 | 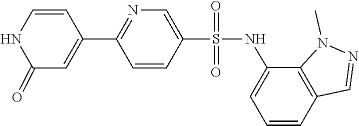 [SMILES: Cn1ncc2cccc(NS(=O)(=O)c3ccc(-c4cc[nH]c(=O)c4)nc3)c21] |
| I-155 | 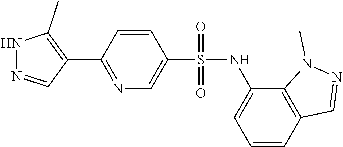 [SMILES: Cc1[nH]ncc1-c1ccc(S(=O)(=O)Nc2cccc3cnn(C)c23)cn1] |
| I-156 | 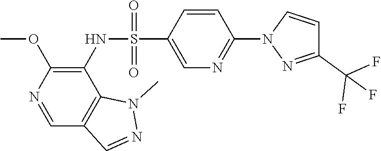 [SMILES: COc1ncc2cnn(C)c2c1NS(=O)(=O)c1ccc(-n2ccc(C(F)(F)F)n2)nc1] |
| I-157 | 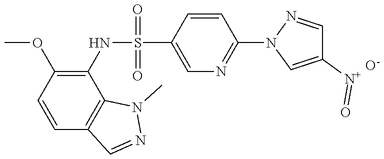 |
| I-158 | 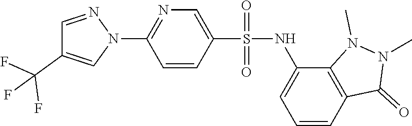 [SMILES: Cn1c(=O)c2cccc(NS(=O)(=O)c3ccc(-n4cc(C(F)(F)F)cn4)nc3)c2n1C] |
| I-159 | 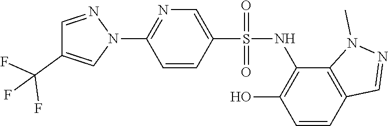 [SMILES: Cn1ncc2ccc(O)c(NS(=O)(=O)c3ccc(-n4cc(C(F)(F)F)cn4)nc3)c21] |
| I-160 | 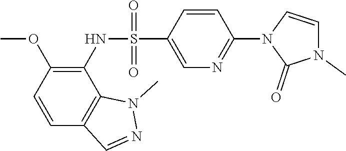 |
| I-161 | 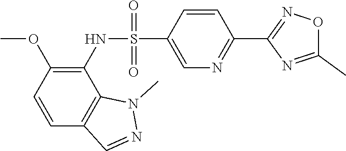 |
| I-162 | 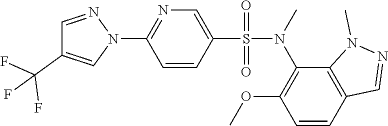 [SMILES: COc1ccc2cnn(C)c2c1N(C)S(=O)(=O)c1ccc(-n2cc(C(F)(F)F)cn2)nc1] |
| I-163 | 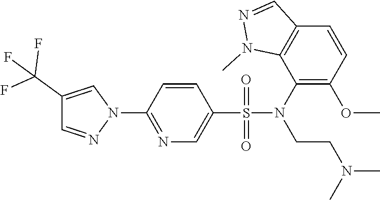 [SMILES: COc1ccc2cnn(C)c2c1N(CCN(C)C)S(=O)(=O)c1ccc(-n2cc(C(F)(F)F)cn2)nc1] |
| I-164 | 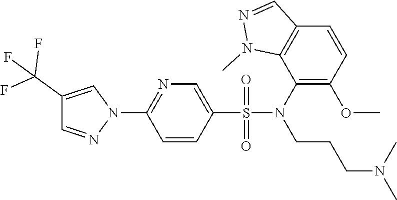 [SMILES: COc1ccc2cnn(C)c2c1N(CCCN(C)C)S(=O)(=O)c1ccc(-n2cc(C(F)(F)F)cn2)nc1] |
| I-165 | 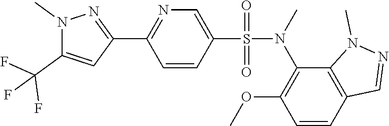 [SMILES: COc1ccc2cnn(C)c2c1N(C)S(=O)(=O)c1ccc(-c2cc(C(F)(F)F)n(C)n2)nc1] |
| I-166 | 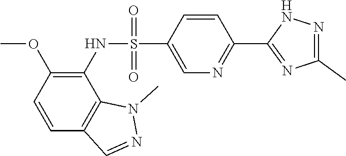 |
| I-167 | 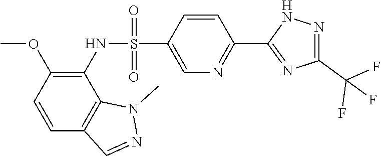 [SMILES: COc1ccc2cnn(C)c2c1NS(=O)(=O)c1ccc(-c2nc(C(F)(F)F)n[nH]2)nc1] |
| I-168 | 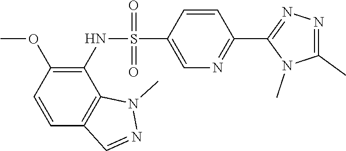 |
| I-169 | 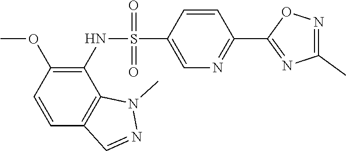 |
| I-170 | 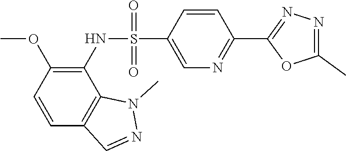 |
| I-171 | 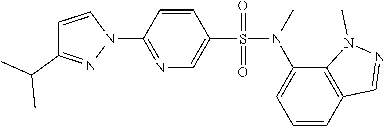 [SMILES: CC(C)c1ccn(-c2ccc(S(=O)(=O)N(C)c3cccc4cnn(C)c34)cn2)n1] |
| I-172 | 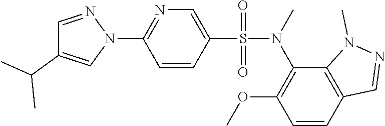 [SMILES: COc1ccc2cnn(C)c2c1N(C)S(=O)(=O)c1ccc(-n2cc(C(C)C)cn2)nc1] |
| I-173 | 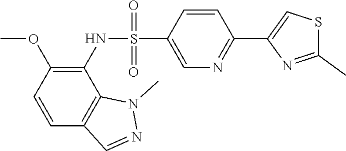 |
| I-174 | 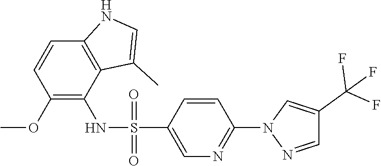 [SMILES: COc1ccc2[nH]cc(C)c2c1NS(=O)(=O)c1ccc(-n2cc(C(F)(F)F)cn2)nc1] |
| I-175 | 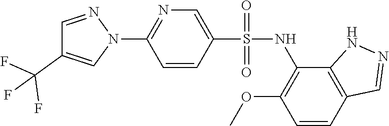 [SMILES: COc1ccc2cn[nH]c2c1NS(=O)(=O)c1ccc(-n2cc(C(F)(F)F)cn2)nc1] |
| I-176 | 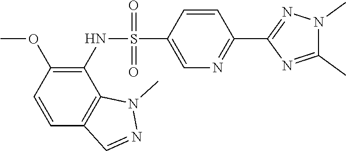 |
| I-177 | 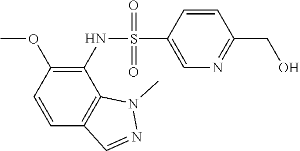 [SMILES: COc1ccc2cnn(C)c2c1NS(=O)(=O)c1ccc(CO)nc1] |
| I-178 | 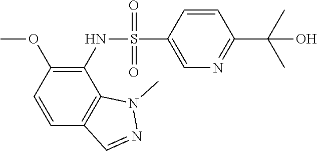 [SMILES: COc1ccc2cnn(C)c2c1NS(=O)(=O)c1ccc(C(C)(C)O)nc1] |
| I-180 | 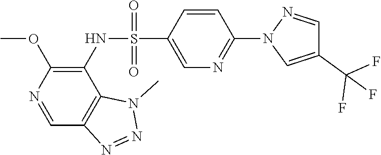 [SMILES: COc1ncc2nnn(C)c2c1NS(=O)(=O)c1ccc(-n2cc(C(F)(F)F)cn2)nc1] |
| I-181 | 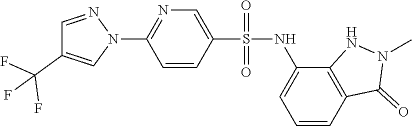 [SMILES: Cn1[nH]c2c(NS(=O)(=O)c3ccc(-n4cc(C(F)(F)F)cn4)nc3)cccc2c1=O] |
| I-182 | 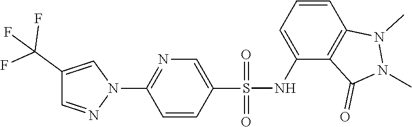 [SMILES: Cn1c(=O)c2c(NS(=O)(=O)c3ccc(-n4cc(C(F)(F)F)cn4)nc3)cccc2n1C] |
| I-183 | 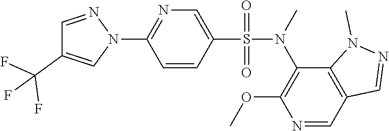 [SMILES: COc1ncc2cnn(C)c2c1N(C)S(=O)(=O)c1ccc(-n2cc(C(F)(F)F)cn2)nc1] |
| I-184 | 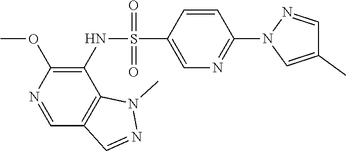 |
| I-185 | 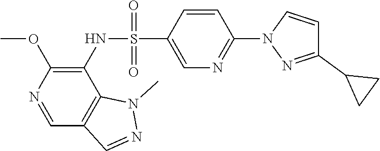 [SMILES: COc1ncc2cnn(C)c2c1NS(=O)(=O)c1ccc(-n2ccc(C3CC3)n2)nc1] |
| I-186 | 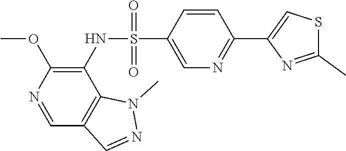 |
| I-187 | 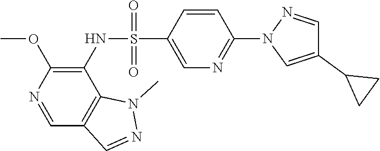 [SMILES: COc1ncc2cnn(C)c2c1NS(=O)(=O)c1ccc(-n2cc(C3CC3)cn2)nc1] |
| I-188 | 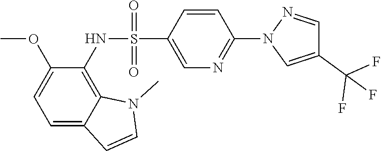 [SMILES: COc1ccc2ccn(C)c2c1NS(=O)(=O)c1ccc(-n2cc(C(F)(F)F)cn2)nc1] |
| I-189 | 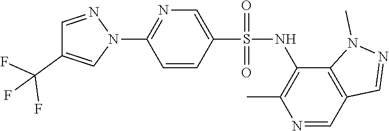 [SMILES: Cc1ncc2cnn(C)c2c1NS(=O)(=O)c1ccc(-n2cc(C(F)(F)F)cn2)nc1] |
| I-190 | 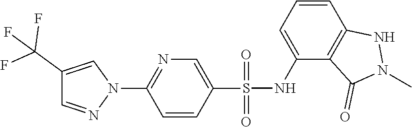 [SMILES: Cn1[nH]c2cccc(NS(=O)(=O)c3ccc(-n4cc(C(F)(F)F)cn4)nc3)c2c1=O] |
| I-191 | 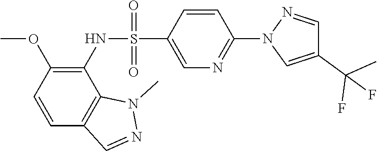 [SMILES: COc1ccc2cnn(C)c2c1NS(=O)(=O)c1ccc(-n2cc(C(C)(F)F)cn2)nc1] |
| I-192 | 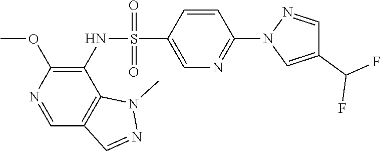 [SMILES: COc1ncc2cnn(C)c2c1NS(=O)(=O)c1ccc(-n2cc(C(F)F)cn2)nc1] |
| I-193 | 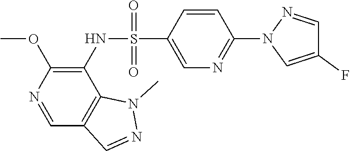 [SMILES: COc1ncc2cnn(C)c2c1NS(=O)(=O)c1ccc(-n2cc(F)cn2)nc1] |
| I-194 | 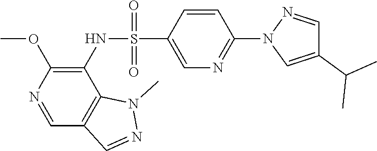 [SMILES: COc1ncc2cnn(C)c2c1NS(=O)(=O)c1ccc(-n2cc(C(C)C)cn2)nc1] |
| I-195 | 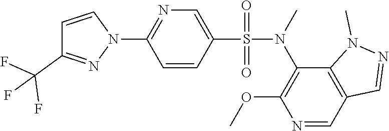 [SMILES: COc1ncc2cnn(C)c2c1N(C)S(=O)(=O)c1ccc(-n2ccc(C(F)(F)F)n2)nc1] |
| I-196 | 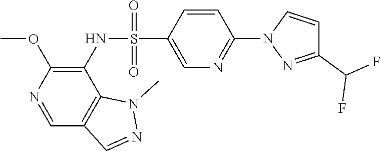 [SMILES: COc1ncc2cnn(C)c2c1NS(=O)(=O)c1ccc(-n2ccc(C(F)F)n2)nc1] |
| I-197 | 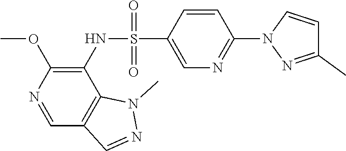 |
| I-198 | 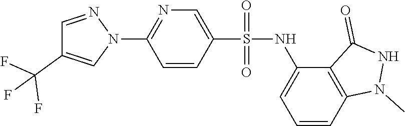 [SMILES: Cn1[nH]c(=O)c2c(NS(=O)(=O)c3ccc(-n4cc(C(F)(F)F)cn4)nc3)cccc21] |
| I-199 | 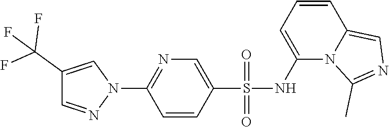 [SMILES: Cc1ncc2cccc(NS(=O)(=O)c3ccc(-n4cc(C(F)(F)F)cn4)nc3)n12] |
| I-200 | 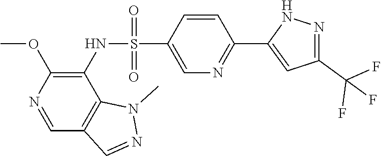 [SMILES: COc1ncc2cnn(C)c2c1NS(=O)(=O)c1ccc(-c2cc(C(F)(F)F)n[nH]2)nc1] |
| I-201 | 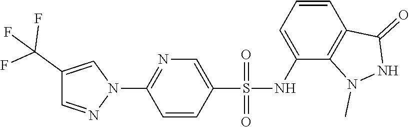 [SMILES: Cn1[nH]c(=O)c2cccc(NS(=O)(=O)c3ccc(-n4cc(C(F)(F)F)cn4)nc3)c21] |
| I-202 | 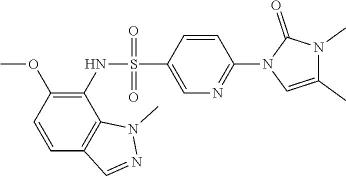 |
| I-204 | 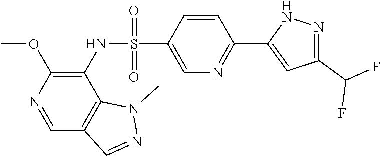 [SMILES: COc1ncc2cnn(C)c2c1NS(=O)(=O)c1ccc(-c2cc(C(F)F)n[nH]2)nc1] |
| I-205 | 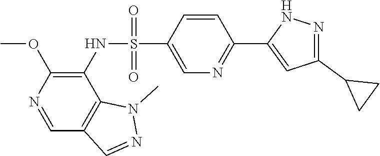 [SMILES: COc1ncc2cnn(C)c2c1NS(=O)(=O)c1ccc(-c2cc(C3CC3)n[nH]2)nc1] |
| I-206 | 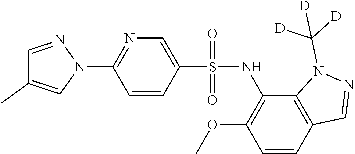 [SMILES: [2H]C([2H])([2H])n1ncc2ccc(OC)c(NS(=O)(=O)c3ccc(-n4cc(C)cn4)nc3)c21] |
| I-207 | 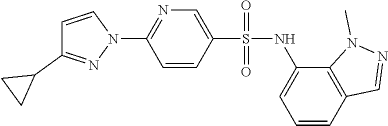 [SMILES: Cn1ncc2cccc(NS(=O)(=O)c3ccc(-n4ccc(C5CC5)n4)nc3)c21] |
| I-208 | 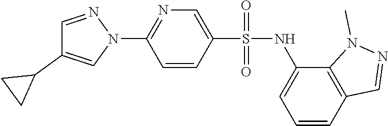 [SMILES: Cn1ncc2cccc(NS(=O)(=O)c3ccc(-n4cc(C5CC5)cn4)nc3)c21] |
| I-209 | 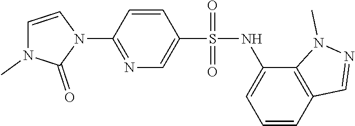 [SMILES: Cn1ccn(-c2ccc(S(=O)(=O)Nc3cccc4cnn(C)c34)cn2)c1=O] |
| I-210 | 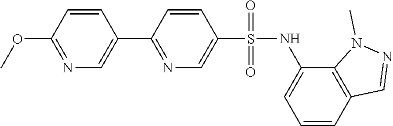 [SMILES: COc1ccc(-c2ccc(S(=O)(=O)Nc3cccc4cnn(C)c34)cn2)cn1] |
| I-211 | 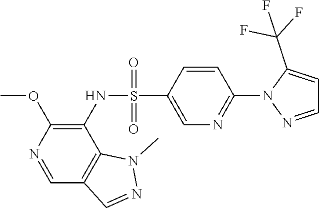 [SMILES: COc1ncc2cnn(C)c2c1NS(=O)(=O)c1ccc(-n2nccc2C(F)(F)F)nc1] |
| I-212 | 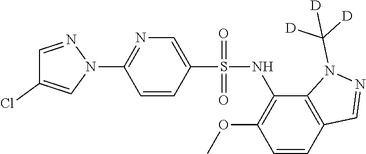 [SMILES: [2H]C([2H])([2H])n1ncc2ccc(OC)c(NS(=O)(=O)c3ccc(-n4cc(Cl)cn4)nc3)c21] |
| I-213 | 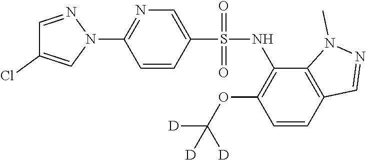 [SMILES: [2H]C([2H])([2H])Oc1ccc2cnn(C)c2c1NS(=O)(=O)c1ccc(-n2cc(Cl)cn2)nc1] |
| I-214 | 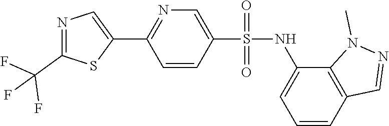 [SMILES: Cn1ncc2cccc(NS(=O)(=O)c3ccc(-c4cnc(C(F)(F)F)s4)nc3)c21] |
| I-215 | 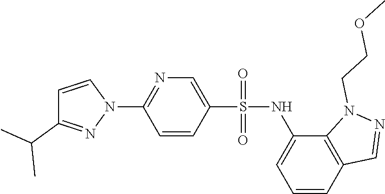 [SMILES: COCCn1ncc2cccc(NS(=O)(=O)c3ccc(-n4ccc(C(C)C)n4)nc3)c21] |
| I-216 | 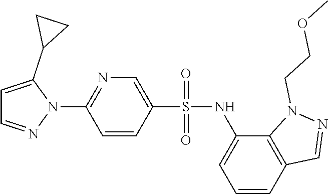 [SMILES: COCCn1ncc2cccc(NS(=O)(=O)c3ccc(-n4nccc4C4CC4)nc3)c21] |
| I-217 | 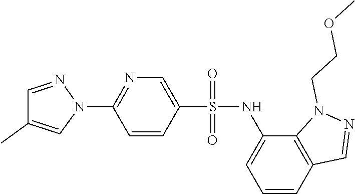 |
| I-218 | 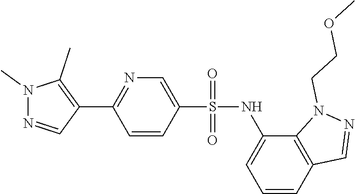 |
| I-219 | 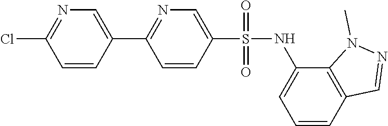 [SMILES: Cn1ncc2cccc(NS(=O)(=O)c3ccc(-c4ccc(Cl)nc4)nc3)c21] |
| I-220 | 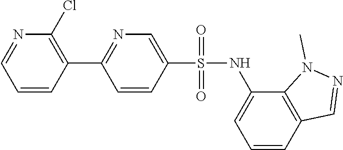 [SMILES: Cn1ncc2cccc(NS(=O)(=O)c3ccc(-c4cccnc4Cl)nc3)c21] |
| I-221 | 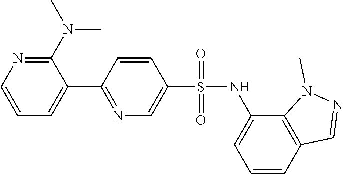 [SMILES: CN(C)c1ncccc1-c1ccc(S(=O)(=O)Nc2cccc3cnn(C)c23)cn1] |
| I-222 | 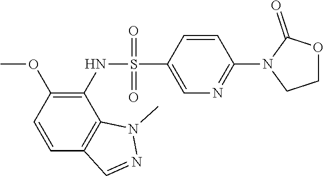 [SMILES: COc1ccc2cnn(C)c2c1NS(=O)(=O)c1ccc(N2CCOC2=O)nc1] |
| I-223 | 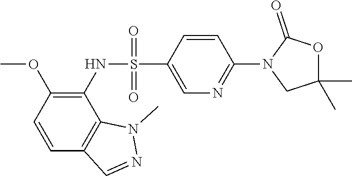 |
| I-224 | 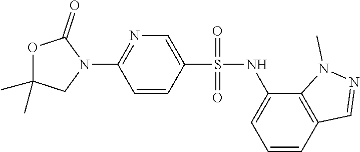 [SMILES: Cn1ncc2cccc(NS(=O)(=O)c3ccc(N4CC(C)(C)OC4=O)nc3)c21] |
| I-225 | 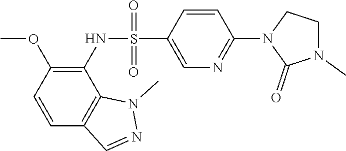 |
| I-226 | 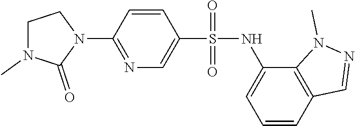 [SMILES: CN1CCN(c2ccc(S(=O)(=O)Nc3cccc4cnn(C)c34)cn2)C1=O] |
| I-227 | 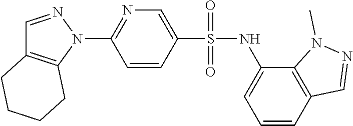 [SMILES: Cn1ncc2cccc(NS(=O)(=O)c3ccc(-n4ncc5c4CCCC5)nc3)c21] |
| I-228 | 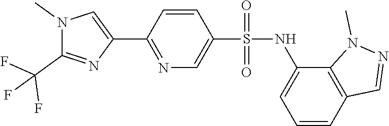 [SMILES: Cn1cc(-c2ccc(S(=O)(=O)Nc3cccc4cnn(C)c34)cn2)nc1C(F)(F)F] |
| I-229 | 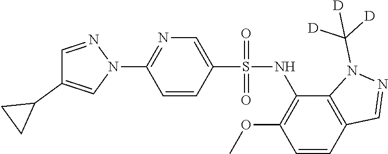 [SMILES: [2H]C([2H])([2H])n1ncc2ccc(OC)c(NS(=O)(=O)c3ccc(-n4cc(C5CC5)cn4)nc3)c21] |
| I-230 | 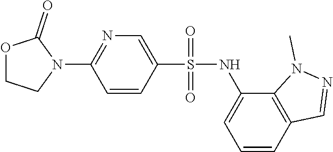 [SMILES: Cn1ncc2cccc(NS(=O)(=O)c3ccc(N4CCOC4=O)nc3)c21] |
| I-231 | 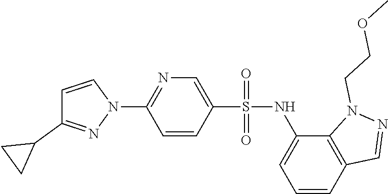 [SMILES: COCCn1ncc2cccc(NS(=O)(=O)c3ccc(-n4ccc(C5CC5)n4)nc3)c21] |
| I-232 | 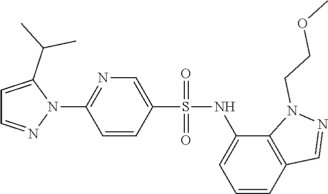 [SMILES: COCCn1ncc2cccc(NS(=O)(=O)c3ccc(-n4nccc4C(C)C)nc3)c21] |
| I-233 | 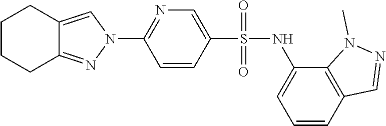 [SMILES: Cn1ncc2cccc(NS(=O)(=O)c3ccc(-n4cc5c(n4)CCCC5)nc3)c21] |
| I-234 | 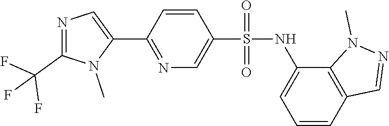 [SMILES: Cn1c(-c2ccc(S(=O)(=O)Nc3cccc4cnn(C)c34)cn2)cnc1C(F)(F)F] |
| I-235 | 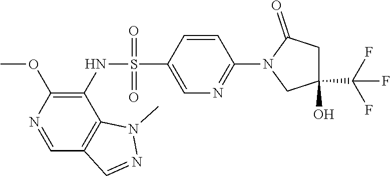 [SMILES: COc1ncc2cnn(C)c2c1NS(=O)(=O)c1ccc(N2C[C@@](O)(C(F)(F)F)CC2=O)nc1] |
| I-236 | 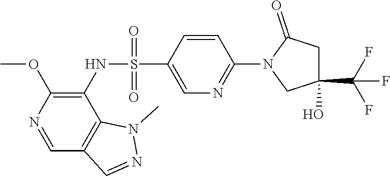 [SMILES: COc1ncc2cnn(C)c2c1NS(=O)(=O)c1ccc(N2C[C@](O)(C(F)(F)F)CC2=O)nc1] |
| I-237 | 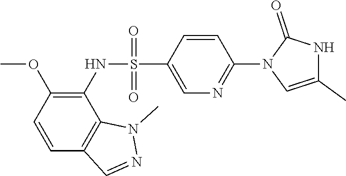 |
| I-238 | 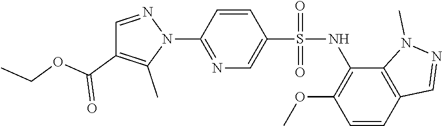 [SMILES: CCOC(=O)c1cnn(-c2ccc(S(=O)(=O)Nc3c(OC)ccc4cnn(C)c34)cn2)c1C] |
| I-239 | 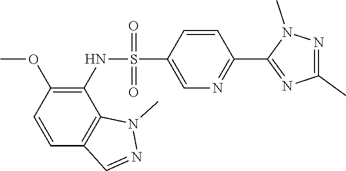 |
| I-240 | 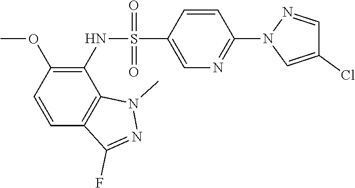 [SMILES: COc1ccc2c(F)nn(C)c2c1NS(=O)(=O)c1ccc(-n2cc(Cl)cn2)nc1] |
| I-241 | 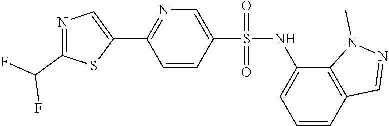 [SMILES: Cn1ncc2cccc(NS(=O)(=O)c3ccc(-c4cnc(C(F)F)s4)nc3)c21] |
| I-242 | 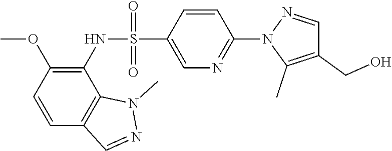 [SMILES: COc1ccc2cnn(C)c2c1NS(=O)(=O)c1ccc(-n2ncc(CO)c2C)nc1] |
| I-243 | 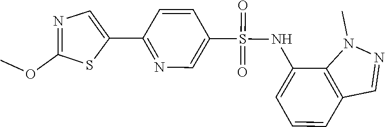 [SMILES: COc1ncc(-c2ccc(S(=O)(=O)Nc3cccc4cnn(C)c34)cn2)s1] |
| I-244 | 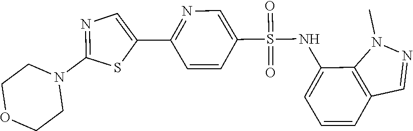 [SMILES: Cn1ncc2cccc(NS(=O)(=O)c3ccc(-c4cnc(N5CCOCC5)s4)nc3)c21] |
| I-245 | 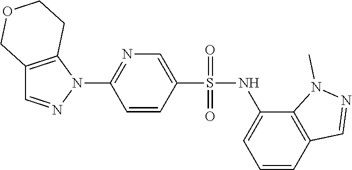 [SMILES: Cn1ncc2cccc(NS(=O)(=O)c3ccc(-n4ncc5c4CCOC5)nc3)c21] |
| I-246 | 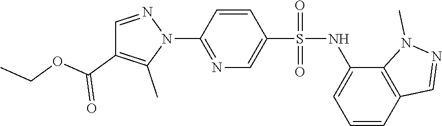 [SMILES: CCOC(=O)c1cnn(-c2ccc(S(=O)(=O)Nc3cccc4cnn(C)c34)cn2)c1C] |
| I-247 | 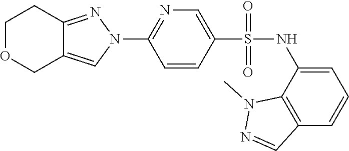 [SMILES: Cn1ncc2cccc(NS(=O)(=O)c3ccc(-n4cc5c(n4)CCOC5)nc3)c21] |
| I-248 | 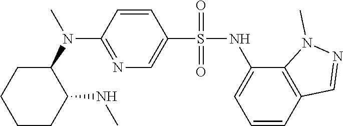 [SMILES: CN[C@@H]1CCCC[C@H]1N(C)c1ccc(S(=O)(=O)Nc2cccc3cnn(C)c23)cn1] |
| I-249 | 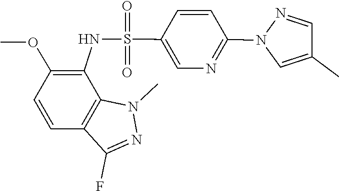 |
| I-250 | 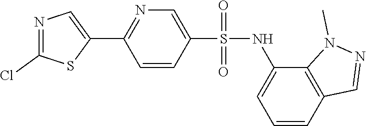 [SMILES: Cn1ncc2cccc(NS(=O)(=O)c3ccc(-c4cnc(Cl)s4)nc3)c21] |
| I-251 | 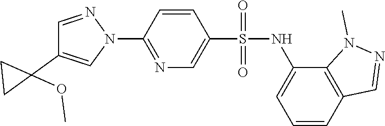 [SMILES: COC1(c2cnn(-c3ccc(S(=O)(=O)Nc4cccc5cnn(C)c45)cn3)c2)CC1] |
| I-252 | 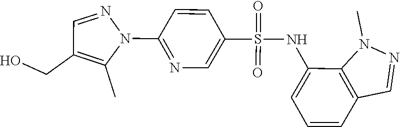 [SMILES: Cc1c(CO)cnn1-c1ccc(S(=O)(=O)Nc2cccc3cnn(C)c23)cn1] |
| I-253 | 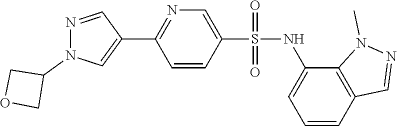 [SMILES: Cn1ncc2cccc(NS(=O)(=O)c3ccc(-c4cnn(C5COC5)c4)nc3)c21] |
| I-254 | 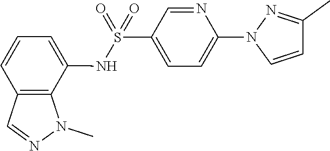 [SMILES: Cc1ccn(-c2ccc(S(=O)(=O)Nc3cccc4cnn(C)c34)cn2)n1] |
| I-255 | 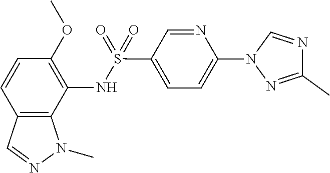 [SMILES: COc1ccc2cnn(C)c2c1NS(=O)(=O)c1ccc(-n2cnc(C)n2)nc1] |
| I-256 | 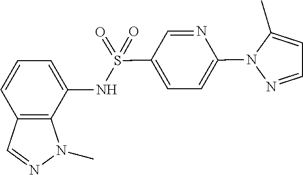 [SMILES: Cc1ccnn1-c1ccc(S(=O)(=O)Nc2cccc3cnn(C)c23)cn1] |
| I-257 | 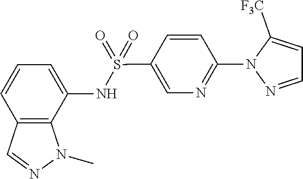 [SMILES: Cn1ncc2cccc(NS(=O)(=O)c3ccc(-n4nccc4C(F)(F)F)nc3)c21] |
| I-258 | 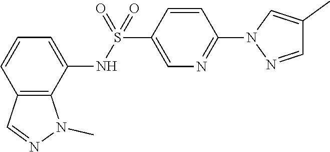 [SMILES: Cc1cnn(-c2ccc(S(=O)(=O)Nc3cccc4cnn(C)c34)cn2)c1] |
| I-259 | 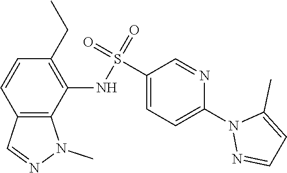 [SMILES: CCc1ccc2cnn(C)c2c1NS(=O)(=O)c1ccc(-n2nccc2C)nc1] |
| I-260 | 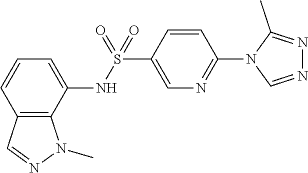 [SMILES: Cc1nncn1-c1ccc(S(=O)(=O)Nc2cccc3cnn(C)c23)cn1] |
| I-261 | 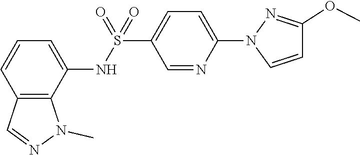 [SMILES: COc1ccn(-c2ccc(S(=O)(=O)Nc3cccc4cnn(C)c34)cn2)n1] |
| I-262 | 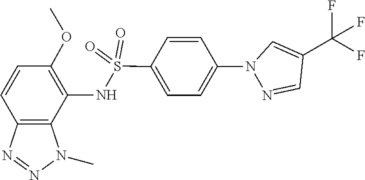 [SMILES: COc1ccc2nnn(C)c2c1NS(=O)(=O)c1ccc(-n2cc(C(F)(F)F)cn2)cc1] |
| I-263 | 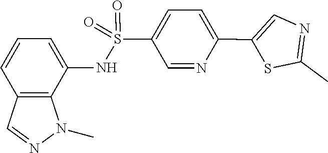 [SMILES: Cc1ncc(-c2ccc(S(=O)(=O)Nc3cccc4cnn(C)c34)cn2)s1] |
| I-264 | 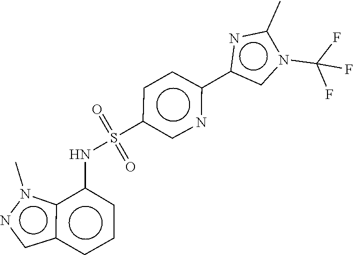 |
| I-265 | 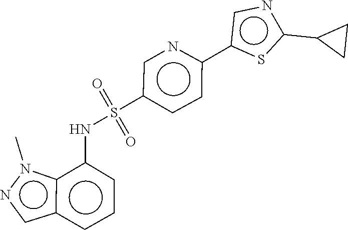 |
| I-266 | 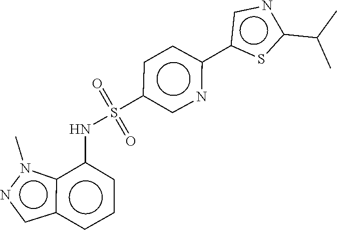 |
| I-267 | 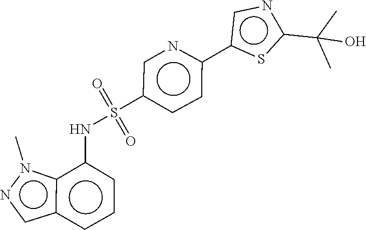 |
| I-268 | 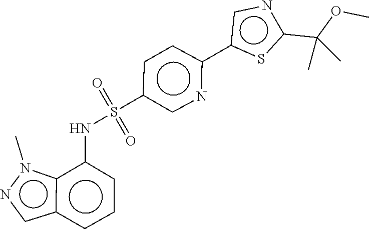 |
| I-269 | 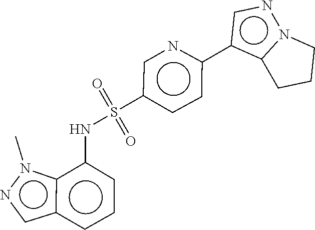 |
| I-270 | 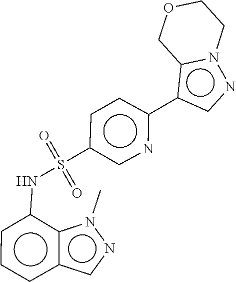 |
| I-271 | 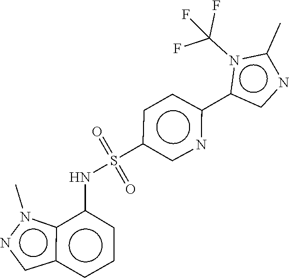 |
| I-272 | 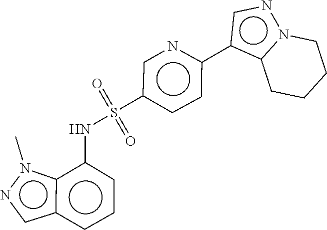 |
| I-273 | 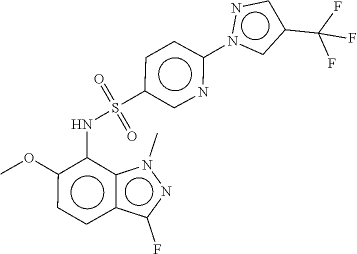 |
| I-274 | 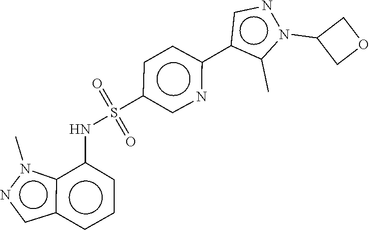 |
| I-275 | 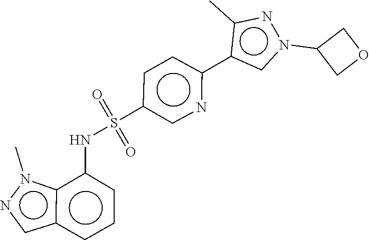 |
| I-276 | 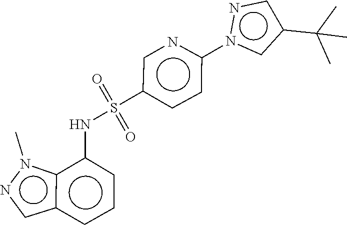 |
| I-277 | 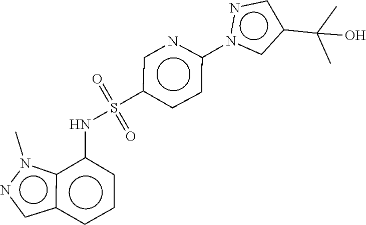 |
| I-278 | 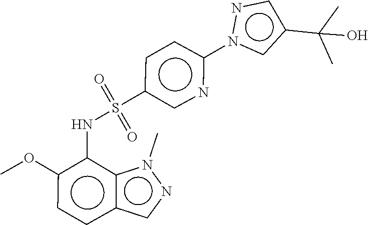 |
| I-279 | 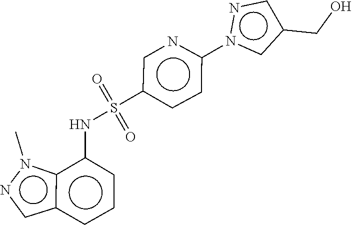 |
| I-280 | 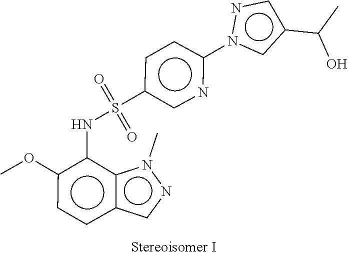 |
| I-281 | 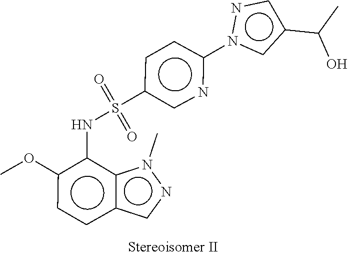 |
| I-282 | 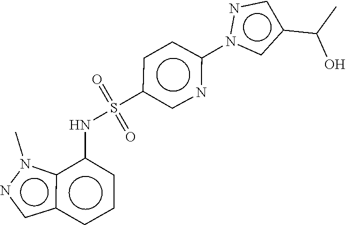 |
| I-283 | 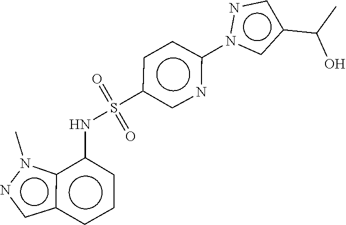 |
| I-284 | 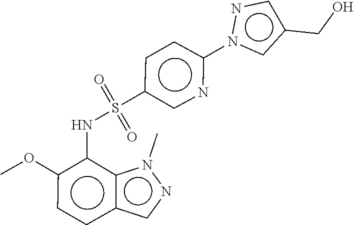 |
| I-285 | 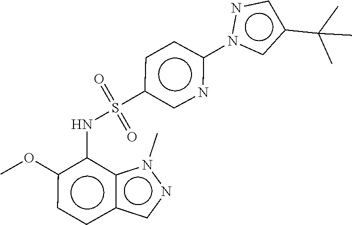 |
| I-286 | 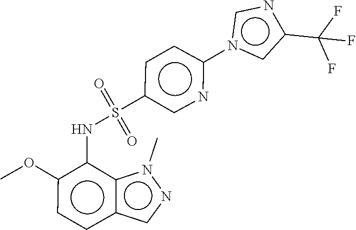 |
| I-287 | 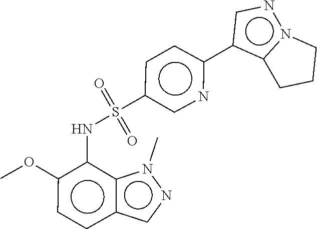 |
| I-288 | 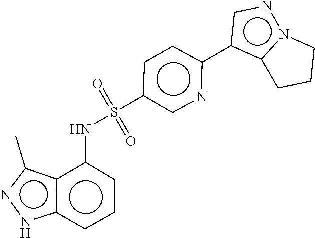 |
| I-289 | 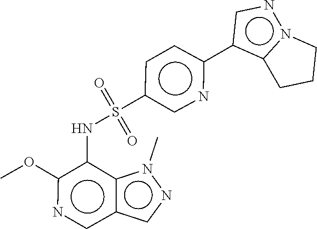 |
| I-290 | 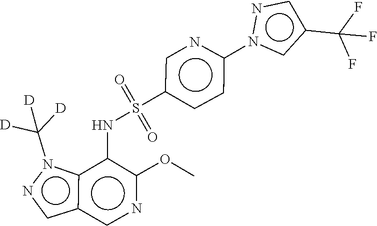 |
| I-291 | 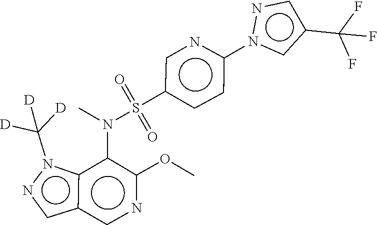 |
| I-292 | 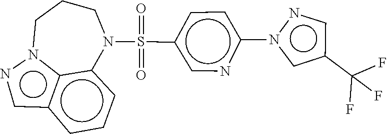 |
| I-293 | 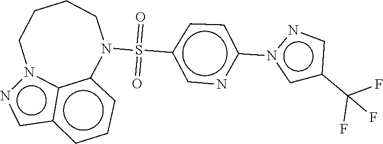 |
| I-294 | 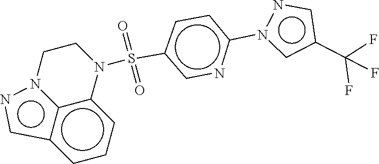 |
| I-295 | 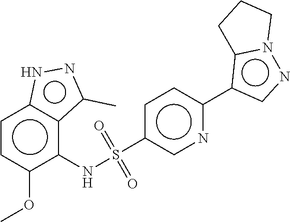 |
| I-296 | 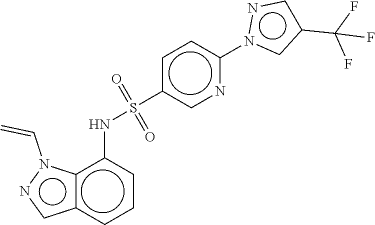 |
| I-297 | 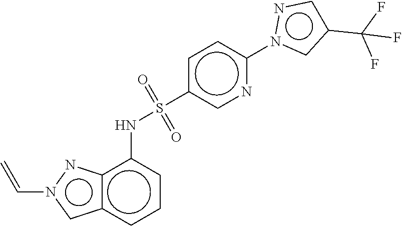 |
| I-298 | 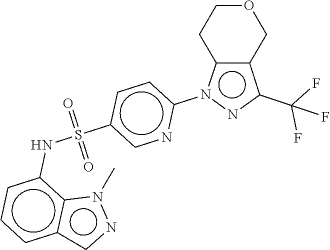 |
| I-299 | 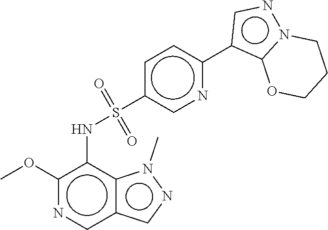 |
| I-300 | 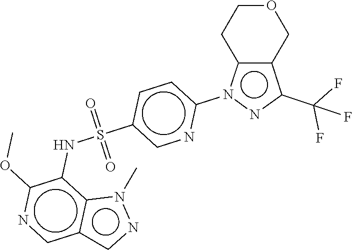 |
| I-301 | 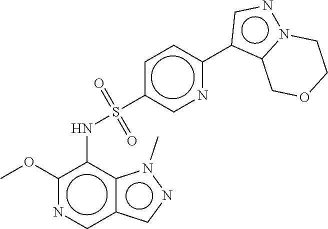 |
| I-302 | 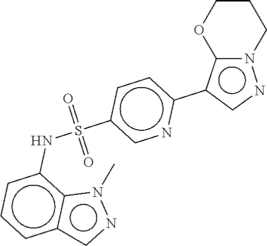 |
| I-303 | 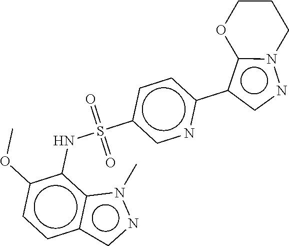 |
| I-304 | 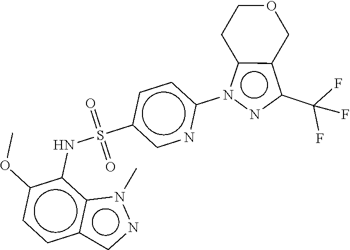 |
| I-305 | 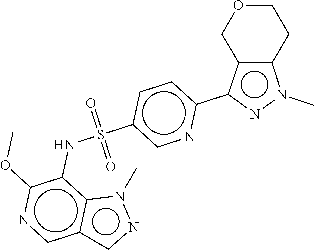 |
| I-306 | 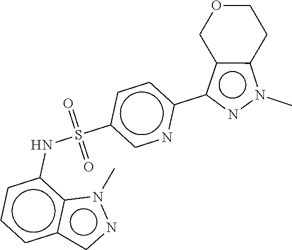 |
| I-307 | 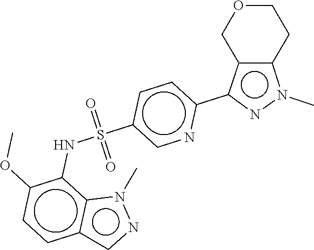 |
| I-308 | 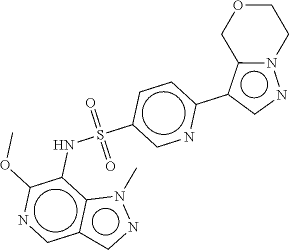 |
| I-309 | 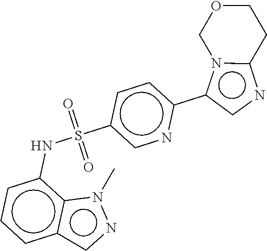 |
| I-310 | 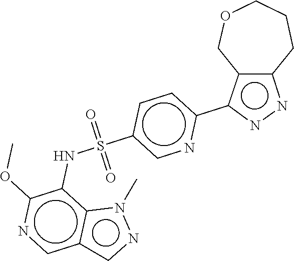 |
| I-311 | 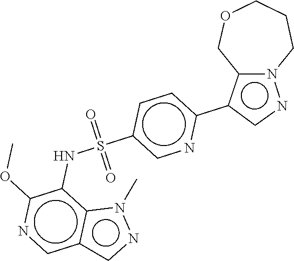 |
| I-312 | 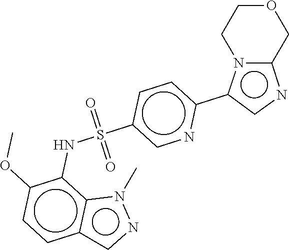 |
| I-313 | 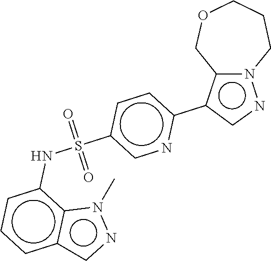 |
| I-314 | 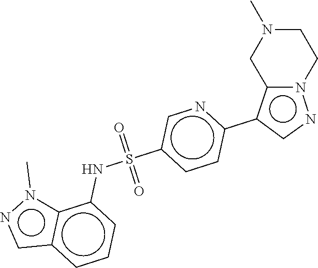 |
| I-315 | 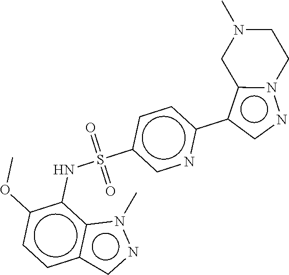 |
| I-316 | 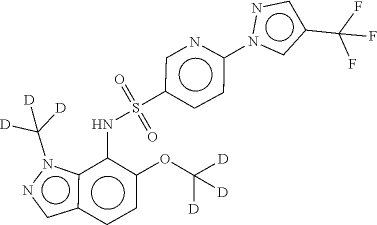 |

### Part G:

Another aspect of the invention provides a compound represented by Formula II:

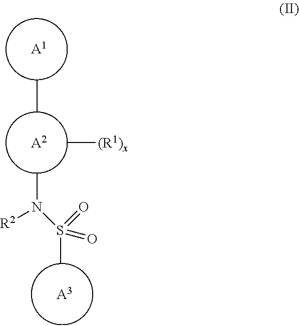

or a pharmaceutically acceptable salt thereof, wherein:
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ are independently hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, nitro, C₃₋₇ halocycloalkyl, C₃₋₇ hydroxycycloalkyl, —O—C₃₋₇ cycloalkyl, —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶;
A² is a pyridinylene, pyridazinylene, or pyrimidinylene;
A³ is

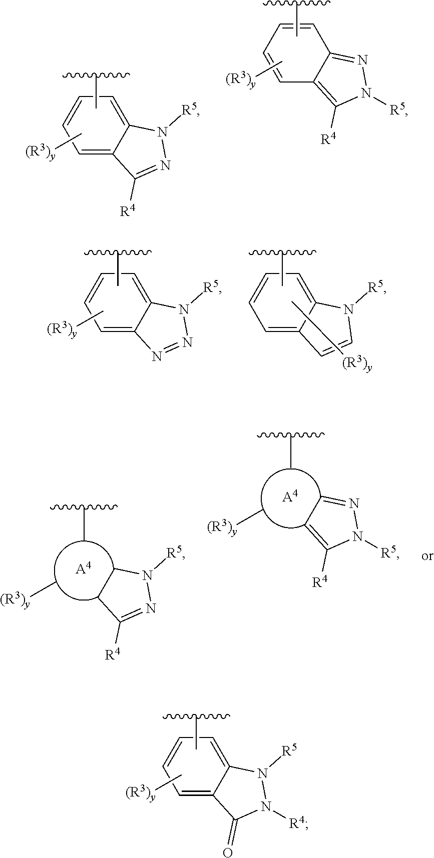

A⁴ is a 6-membered aromatic ring containing 1 nitrogen atom;
y is 1 or 2; and
m, n, and x are independently 0, 1, or 2;
provided at least one occurrence of R³ is C₁₋₆ alkoxyl, C₁₋₆ deuteroalkoxyl, or C₃₋₇ cycloalkyl.

The definitions of variables in Formula II above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula II.

As defined generally above, R¹ represents independently for each occurrence halo, C₁₋₄ alkyl, C₁₋₄ haloalkyl, or cyano. In certain embodiments, R¹ is halo. In certain embodiments, R¹ is F. In certain embodiments, R¹ is Cl. In certain embodiments, R¹ is Br. In certain embodiments, R¹ is I. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is C₁₋₄ haloalkyl. In certain embodiments, R¹ is C₁ haloalkyl. In certain embodiments, R¹ is C₂ haloalkyl. In certain embodiments, R¹ is C₃ haloalkyl. In certain embodiments, R¹ is C₄ haloalkyl. In certain embodiments, R¹ is cyano. In certain embodiments, R¹ is selected from the groups depicted in the compounds in Table 2 below.

As defined generally above, R² is hydrogen, C₁₋₄ alkyl, C₂₋₄ hydroxyalkyl, or —(C₁₋₆ alkylene)-N(R⁸)(R⁹). In certain embodiments, R² is hydrogen. In certain embodiments, R² is C₁₋₄ alkyl. In certain embodiments, R² is C₁ alkyl. In certain embodiments, R² is C₂ alkyl. In certain embodiments, R² is C₃ alkyl. In certain embodiments, R² is C₄ alkyl. In certain embodiments, R² is C₂₋₄ hydroxyalkyl. In certain embodiments, R² is C₂ hydroxyalkyl. In certain embodiments, R² is C₃ hydroxyalkyl. In certain embodiments, R² is C₄ hydroxyalkyl. In certain embodiments, R² is —(C₁₋₆ alkylene)-N(R⁸)(R⁹). In certain embodiments, R² is selected from the groups depicted in the compounds in Table 2 below.

As defined generally above, R⁵ is hydrogen, C₁₋₄ alkyl, or C₁₋₄ deuteroalkyl. In certain embodiments, R⁵ is hydrogen. In certain embodiments, R⁵ is C₁₋₄ alkyl. In certain embodiments, R⁵ is C₁ alkyl. In certain embodiments, R⁵ is C₂ alkyl. In certain embodiments, R⁵ is C₃ alkyl. In certain embodiments, R⁵ is C₄ alkyl. In certain embodiments, R⁵ is C₁₋₄ deuteroalkyl. In certain embodiments, R⁵ is C₁ deuteroalkyl. In certain embodiments, R⁵ is C₂ deuteroalkyl. In certain embodiments, R⁵ is C₃ deuteroalkyl. In certain embodiments, R⁵ is C₄ deuteroalkyl. In certain embodiments, R⁵ is selected from the groups depicted in the compounds in Table 2 below.

As defined generally above, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur. In certain embodiments, R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰. In certain embodiments, R³ is C₁₋₆ alkyl. In certain embodiments, R³ is ethyl. In certain embodiments, R³ is C₃₋₇ cycloalkyl. In certain embodiments, R³ is cyclopropyl. In certain embodiments, R³ is C₁₋₆ alkoxyl. In certain embodiments, R³ is methoxy. In certain embodiments, R³ is halo. In certain embodiments, R³ is hydroxyl. In certain embodiments, R³ is C₁₋₆ haloalkyl. In certain embodiments, R³ is C₁₋₆ deuteroalkyl. In certain embodiments, R³ is C₁₋₆ deuteroalkoxyl, In certain embodiments, R³ is —O—C₃₋₇ cycloalkyl. In certain embodiments, R³ is cyano. In certain embodiments, R³ is —CO₂R¹⁰. In certain embodiments, R³ is —C(O)N(R⁸)(R⁹). In certain embodiments, R³ is —N(R⁸)C(O)R¹⁰. In certain embodiments, R³ is —S(O₂)R¹⁰. In certain embodiments, two occurrences of R³ are taken together with the intervening atoms to form a 5-7 membered ring containing 0, 1, or 2 heteroatoms independently selected from nitrogen, oxygen, and sulfur. In certain embodiments, R³ is selected from the groups depicted in the compounds in Table 2 below.

As defined generally above, R⁴ is hydrogen, halo, or C₁₋₄ alkyl. In certain embodiments, R⁴ is hydrogen. In certain embodiments, R⁴ is halo. In certain embodiments, R⁴ is C₁₋₄ alkyl. In certain embodiments, R⁴ is C₁ alkyl. In certain embodiments, R⁴ is C₂ alkyl. In certain embodiments, R⁴ is C₃ alkyl. In certain embodiments, R⁴ is C₄ alkyl. In certain embodiments, R⁴ is selected from the groups depicted in the compounds in Table 2 below.

As defined generally above, R⁶ represents independently for each occurrence halo, hydroxyl, C₁₋₆ alkyl, C₁₋₆ haloalkyl, cyano, C₁₋₆ alkoxyl, C₃₋₇ cycloalkyl, C₃₋₇halocycloalkyl, C₃₋₇ hydroxycycloalkyl, —O—C₃₋₇ cycloalkyl, nitro, —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo. In certain embodiments, R⁶ is C₁₋₆ haloalkyl. In certain embodiments, R⁶ is —CF₃. In certain embodiments, R⁶ is C₃₋₇ cycloalkyl. In certain embodiments, R⁶ is cyclopropyl. In certain embodiments, R⁶ is C₃₋₇ halocycloalkyl. In certain embodiments, R⁶ is C₃₋₇ hydroxycycloalkyl. In certain embodiments, R⁶ is halo. In certain embodiments, R⁶ represents independently for each occurrence C₁₋₆ haloalkyl, —C(O)R⁷, or —C(O)N(R⁸)(R⁹). In certain embodiments, R⁶ is hydroxyl. In certain embodiments, R⁶ is C₁₋₆ alkyl. In certain embodiments, R⁶ is cyano. In certain embodiments, R⁶ is C₁₋₆ alkoxyl. In certain embodiments, R⁶ is —O—C₃₋₇ cycloalkyl. In certain embodiments, R⁶ is —C(O)R⁷. In certain embodiments, R⁶ is —C(O)N(R⁸)(R⁹). In certain embodiments, R⁶ is —N(R⁸)C(O)R¹⁰. In certain embodiments, R⁶ is —S(O₂)R¹⁰. In certain embodiments, R⁶ is —S(O₂)N(R⁸)(R⁹). In certain embodiments, R⁶ is —N(R⁸)S(O₂)R¹⁰. In certain embodiments, R⁶ is a 3-7 membered saturated heterocyclyl containing 1 or 2 heteroatoms independently selected from oxygen and nitrogen, wherein the heterocyclyl is substituted with 0 or 1 halo. In certain embodiments, R⁶ is a 3-7 membered saturated heterocyclyl containing 1 heteroatom selected from oxygen and nitrogen, wherein the heterocyclyl is substituted with 0 or 1 halo. In certain embodiments, R⁶ is a 3-7 membered saturated heterocyclyl containing 2 heteroatoms independently selected from oxygen and nitrogen, wherein the heterocyclyl is substituted with 0 or 1 halo. In certain embodiments, R⁶ is nitro. In certain embodiments, R⁶ is selected from the groups depicted in the compounds in Table 2 below.

As defined generally above, R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is —OH. In certain embodiments, R⁷ is —OH. In certain embodiments, R⁷ is —O—(C₁₋₆ alkyl). In certain embodiments, R⁷ is —O—C₃₋₇ cycloalkyl. In certain embodiments, R⁷ is a 4-6 membered saturated heterocyclyl containing 1 or 2 heteroatoms independently selected from oxygen, nitrogen, and sulfur, wherein the heterocyclyl is substituted with m occurrences of R¹¹—O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 4-membered saturated heterocyclyl containing 1 or 2 heteroatoms independently selected from oxygen, nitrogen, and sulfur, wherein the heterocyclyl is substituted with m occurrences of R¹¹—O—(C₁₋₆ alkyl), —O—C₃₋₇ cycloalkyl, or a 4-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 5-membered saturated heterocyclyl containing 1 or 2 heteroatoms independently selected from oxygen, nitrogen, and sulfur, wherein the heterocyclyl is substituted with m occurrences of R¹¹—O—(C₁₋₆ alkyl), —O—C₃₋₇ cycloalkyl, or a 5-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is a 6-membered saturated heterocyclyl containing 1 or 2 heteroatoms independently selected from oxygen, nitrogen, and sulfur, wherein the heterocyclyl is substituted with m occurrences of R¹¹—O—(C₁₋₆ alkyl), —O—C₃₋₇ cycloalkyl, or a 6-membered saturated heterocyclyl containing 1 or 2 heteroatoms independently selected from oxygen, nitrogen, and sulfur, wherein the heterocyclyl is substituted with m occurrences of R¹¹. In certain embodiments, R⁷ is selected from the groups depicted in the compounds in Table 2 below.

As defined generally above, R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom. In certain embodiments, R⁸ is hydrogen. In certain embodiments, R⁸ is C₁₋₆ alkyl. In certain embodiments, R⁸ is C₃₋₇ cycloalkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁸ and R⁹ are taken together with the nitrogen atom to which they are attached to form a 3-7 membered heterocyclic ring containing 1 nitrogen atom. In certain embodiments, R⁸ is selected from the groups depicted in the compounds in Table 2 below. In certain embodiments, R⁹ is selected from the groups depicted in the compounds in Table 2 below.

As defined generally above, R¹⁰ represents independently for each occurrence C₁₋₆ alkyl or —(C₀₋₅ alkylene)-C₃₋₇ cycloalkyl. In certain embodiments, R¹⁰ is C₁₋₆ alkyl. In certain embodiments, R¹⁰ is —(C₀₋₅ alkylene)-C₃₋₇ cycloalkyl. In certain embodiments, R¹⁰ is selected from the groups depicted in the compounds in Table 2 below.

As defined generally above, R¹¹ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R¹¹ is halo. In certain embodiments, R¹¹ is hydroxyl. In certain embodiments, R¹¹ is C₁₋₆ alkyl. In certain embodiments, R¹¹ is C₁₋₆ haloalkyl. In certain embodiments, R¹¹ is C₁₋₆ alkoxyl. In certain embodiments, R¹¹ is C₃₋₇ cycloalkyl. In certain embodiments, R¹¹ is selected from the groups depicted in the compounds in Table 2 below.

As defined generally above, A¹ is a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2 or 3 heteroatoms independently selected from oxygen, nitrogen, and sulfur, wherein the heteroaryl and heterocyclyl are substituted with n occurrences of R⁶. In certain embodiments, A¹ is a 5 membered heteroaryl containing 1, 2, or 3 heteroatoms independently selected from oxygen, nitrogen, and sulfur, wherein the heteroaryl is substituted with n occurrences of R⁶. In certain embodiments, A¹ is pyrazolyl, imidazolyl, oxazolyl, isoxazolyl, isothiazolyl, pyrrolyl, or furanyl, each of which is substituted with n occurrences of R⁶. In certain embodiments, A¹ is pyrazolyl substituted with n occurrences of R⁶. In certain embodiments, A¹ is

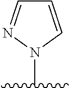 [SMILES: *n1cccn1]

substituted with n occurrences of R⁶. In certain embodiments, A¹ is

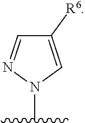

In certain embodiments, A¹ is a 5-membered heteroaryl containing 1 heteratom independently selected from oxygen, nitrogen, and sulfur, wherein the heteroaryl is substituted with n occurrences of R⁶. In certain embodiments, A¹ is a 5-membered heteroaryl containing 2 heteroatoms independently selected from oxygen, nitrogen, and sulfur, wherein the heteroaryl is substituted with n occurrences of R⁶. In certain embodiments, A¹ is a 5-membered heteroaryl containing 3 heteroatoms independently selected from oxygen, nitrogen, and sulfur, wherein the heteroaryl is substituted with n occurrences of R⁶.

In certain embodiments, A¹ is a 3-10 membered saturated heterocyclyl containing 1, 2 or 3 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 1 heteroatom selected from oxygen, nitrogen, and sulfur, wherein the heterocyclyl is substituted with n occurrences of R⁶. In certain embodiments, A¹ is a 3-10 membered saturated heterocyclyl containing 2 heteroatoms independently selected from oxygen, nitrogen, and sulfur, wherein the heterocyclyl is substituted with n occurrences of R⁶. In certain embodiments, A¹ is a 3-10 membered saturated heterocyclyl containing 3 heteroatoms independently selected from oxygen, nitrogen, and sulfur, wherein the heterocyclyl is substituted with n occurrences of R⁶.

In certain embodiments, A¹ is a 9-10 membered bicyclic heteroaryl containing 1, 2, or 3 heteroatoms independently selected from oxygen, nitrogen, and sulfur, wherein the heteroaryl is substituted with n occurrences of R⁶.

In certain embodiments, A¹ is selected from the groups depicted in the compounds in Table 2 below.

As defined generally above, A² is a pyridinylene, pyridazinylene, or pyrimidinylene. In certain embodiments, A² is a pyridinylene. In certain embodiments, A² is pyridazinylene or pyrimidinylene. In certain embodiments, A² is pyridazinylene. In certain embodiments, A² is pyrimidinylene. In certain embodiments, A² is selected from the groups depicted in the compounds in Table 2 below.

As defined generally above, A³ is

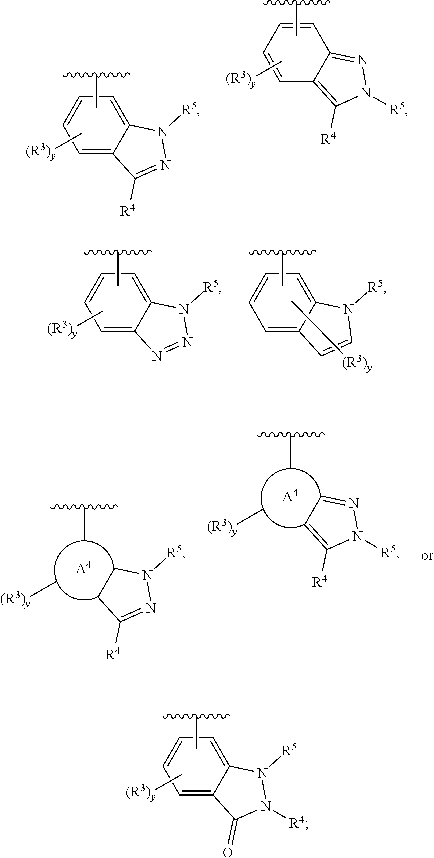

In certain embodiments, A³ is

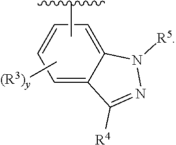

In certain embodiments, A³ is

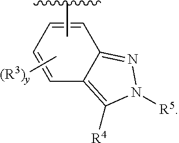

In certain embodiments, A³ is

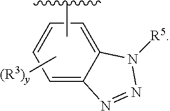

In certain embodiments, A³ is

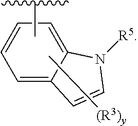

In certain embodiments, A³ is

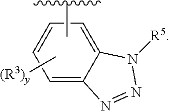

In certain embodiments, A³ is

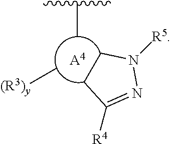

In certain embodiments, A³ is

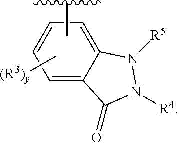

In certain embodiments, A³ is selected from the groups depicted in the compounds in Table 2 below.

As defined generally above, A⁴ is a 6-membered aromatic ring containing 1 nitrogen atom.

As defined generally above, y is 1 or 2. In certain embodiments, y is 1. In certain embodiments, y is 2. In certain embodiments, y is selected from the corresponding value in the groups depicted in the compounds in Table 2 below.

As defined generally above, m, n, and x are independently 0, 1, or 2. In certain embodiments, x is 0. In certain embodiments, n is 1. In certain embodiments, n is 2. In certain embodiments, m is 0. In certain embodiments, m is 1. In certain embodiments, m is 2. In certain embodiments, n is 0. In certain embodiments, x is 1. In certain embodiments, x is 2. In certain embodiments, m is selected from the corresponding value in the groups depicted in the compounds in Table 2 below. In certain embodiments, n is selected from the corresponding value in the groups depicted in the compounds in Table 2 below. In certain embodiments, x is selected from the corresponding value in the groups depicted in the compounds in Table 2 below. PART H:

Another aspect of the invention provides a compound in Table 2 below, or a pharmaceutically acceptable salt thereof. In certain embodiments, the compound is a compound in Table 2. In certain embodiments, the compound is any one of compounds II-1 to II-17 in Table 2 below, or a pharmaceutically acceptable salt thereof. In certain embodiments, the compound is any one of compounds II-1 to II-17 in Table 2 below. In certain embodiments, the compound is compound II-18 in Table 2 below, or a pharmaceutically acceptable salt thereof. In certain embodiments, the compound is compound II-18 in Table 2 below.

TABLE 2

| Compound | |
|---|---|
| No. | Structure |
| II-1 | 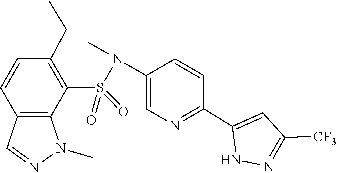 [SMILES: CCc1ccc2cnn(C)c2c1S(=O)(=O)N(C)c1ccc(-c2cc(C(F)(F)F)n[nH]2)nc1] |
| II-2 | 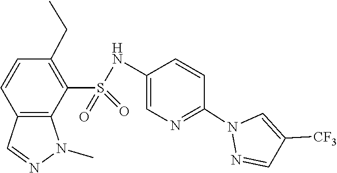 [SMILES: CCc1ccc2cnn(C)c2c1S(=O)(=O)Nc1ccc(-n2cc(C(F)(F)F)cn2)nc1] |
| II-3 | 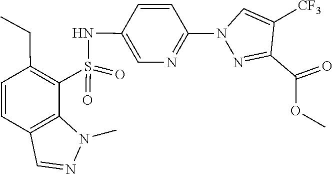 [SMILES: CCc1ccc2cnn(C)c2c1S(=O)(=O)Nc1ccc(-n2cc(C(F)(F)F)c(C(=O)OC)n2)nc1] |
| II-4 | 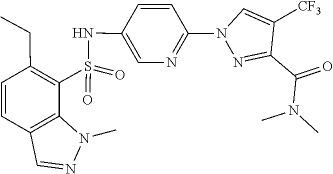 [SMILES: CCc1ccc2cnn(C)c2c1S(=O)(=O)Nc1ccc(-n2cc(C(F)(F)F)c(C(=O)N(C)C)n2)nc1] |
| II-5 | 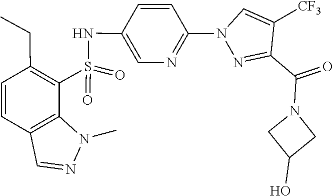 [SMILES: CCc1ccc2cnn(C)c2c1S(=O)(=O)Nc1ccc(-n2cc(C(F)(F)F)c(C(=O)N3CC(O)C3)n2)nc1] |
| II-6 | 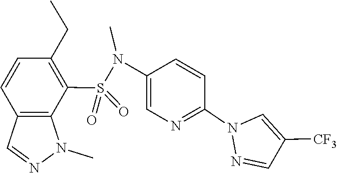 [SMILES: CCc1ccc2cnn(C)c2c1S(=O)(=O)N(C)c1ccc(-n2cc(C(F)(F)F)cn2)nc1] |
| II-7 | 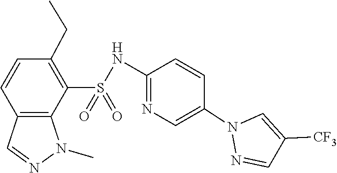 [SMILES: CCc1ccc2cnn(C)c2c1S(=O)(=O)Nc1ccc(-n2cc(C(F)(F)F)cn2)cn1] |
| II-8 | 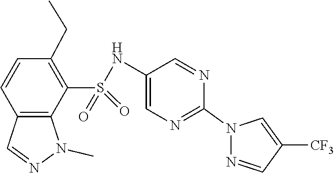 [SMILES: CCc1ccc2cnn(C)c2c1S(=O)(=O)Nc1cnc(-n2cc(C(F)(F)F)cn2)nc1] |
| II-9 | 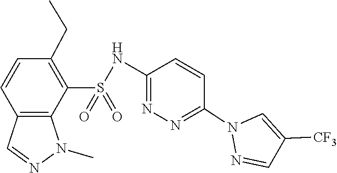 [SMILES: CCc1ccc2cnn(C)c2c1S(=O)(=O)Nc1ccc(-n2cc(C(F)(F)F)cn2)nn1] |
| II-10 | 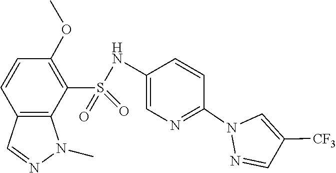 [SMILES: COc1ccc2cnn(C)c2c1S(=O)(=O)Nc1ccc(-n2cc(C(F)(F)F)cn2)nc1] |
| II-11 | 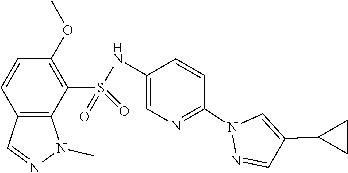 [SMILES: COc1ccc2cnn(C)c2c1S(=O)(=O)Nc1ccc(-n2cc(C3CC3)cn2)nc1] |
| II-12 | 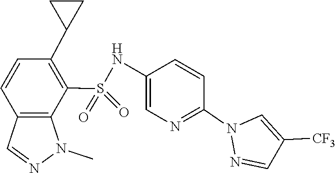 [SMILES: Cn1ncc2ccc(C3CC3)c(S(=O)(=O)Nc3ccc(-n4cc(C(F)(F)F)cn4)nc3)c21] |
| II-13 | 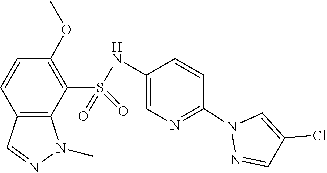 [SMILES: COc1ccc2cnn(C)c2c1S(=O)(=O)Nc1ccc(-n2cc(Cl)cn2)nc1] |
| II-14 | 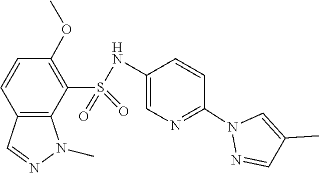 [SMILES: COc1ccc2cnn(C)c2c1S(=O)(=O)Nc1ccc(-n2cc(C)cn2)nc1] |
| II-15 | 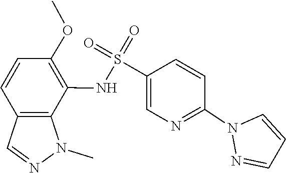 [SMILES: COc1ccc2cnn(C)c2c1NS(=O)(=O)c1ccc(-n2cccn2)nc1] |
| II-16 | 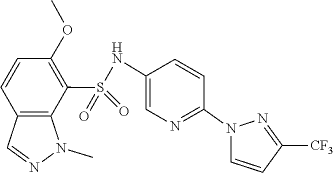 [SMILES: COc1ccc2cnn(C)c2c1S(=O)(=O)Nc1ccc(-n2ccc(C(F)(F)F)n2)nc1] |
| II-17 | 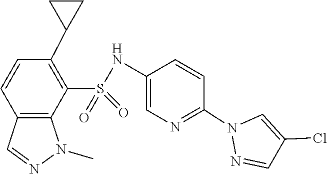 [SMILES: Cn1ncc2ccc(C3CC3)c(S(=O)(=O)Nc3ccc(-n4cc(Cl)cn4)nc3)c21] |
| II-18 | 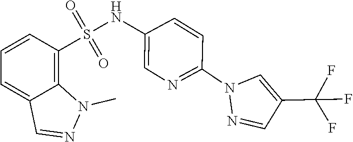 [SMILES: Cn1ncc2cccc(S(=O)(=O)Nc3ccc(-n4cc(C(F)(F)F)cn4)nc3)c21] |

Methods for preparing compounds described herein are illustrated in the following synthetic scheme. The scheme is provided for the purpose of illustrating the invention, and is not intended to limit the scope or spirit of the invention. Starting materials shown in the scheme can be obtained from commercial sources or can be prepared based on procedures described in the literature.

In the scheme, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in Protecting Groups in Organic Synthesis, 3^{rd} Edition, T. W. Greene and P. G. M. Wuts, John Wiley & Sons, 1999 and Greene's Protective Groups in Organic Synthesis, 5th Ed., (Peter G. M. Wuts, John Wiley & Sons: 2014), the entire contents of both of which are hereby incorporated by reference.

The synthetic route illustrated in Scheme 1 is a general method for preparing pyridinylsulfonamides E. Reaction of pyridinyl sulfonylchloride A with amine B provides sulfonamide C. Reaction of sulfonamide C with pyrazole D provides pyridinylsulfonamide E.

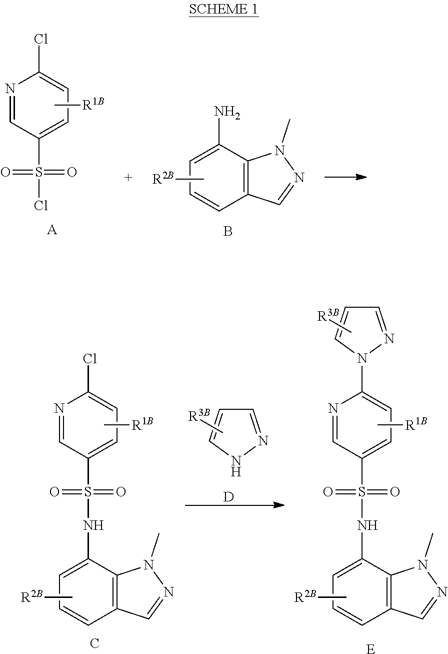

The modular synthetic route illustrated in Scheme 1 can be adjusted to provide additional pyridinylsulfonamide compounds by conducting functional group transformations on the intermediate and final compounds. Such functional group transformations are well known in the art, as described in, for example, Comprehensive Organic Synthesis (B. M. Trost & I. Fleming, eds., 1991-1992); Organic Synthesis, 3^{rd} Ed. (Michael B. Smith, Wavefunction, Inc., Irvine: 2010); Modern Methods of Organic Synthesis, 4^{th} Ed. (William Carruthers and lain Coldham, Cambridge University Press, Cambridge: 2004); March's Advanced Organic Chemistry: Reactions, Mechanisms, and Structure, 8^{th} Ed., (Michael B. Smith, John Wiley & Sons, New York: 2020); and Comprehensive Organic Transformations: A Guide to Functional Group Preparations, 3rd Ed. (Richard C. Larock, ed., John Wiley & Sons, New York: 2018).

### II. THERAPEUTIC APPLICATIONS OF PYRIDINYLSULFONAMIDE COMPOUNDS

Compounds described herein are useful for treating a disease or condition mediated by MALT1. Exemplary diseases or conditions mediated by MALT1 include proliferative disorders (e.g., cancer, neoplasia), inflammatory disorders (e.g., chronic inflammatory disorder, acute inflammatory disorder, auto-inflammatory disorder), autoimmune disorders, fibrotic disorders, metabolic disorders, cardiovascular disorders, cerebrovascular disorders, and myeloid cell-driven hyper-inflammatory responses in COVID-19 infections.

Accordingly, one aspect of the invention provides a method of treating a disease or condition mediated by MALT1 in a subject. The method comprises administering to a subject in need thereof a therapeutically effective amount of a compound described herein, such as a compound of Formula I, I-A, I-B, I-C, II, or other compounds in section I, to treat the disease or condition. In certain embodiments, the compound is a compound of Formula Ia, Ib, Ic, Id or defined by one of the embodiments described above. Further description of exemplary diseases or conditions mediated by MALT1 is provided herein below.

Another aspect of the invention provides a method of inhibiting the activity of MALT1. The method comprises contacting a MALT1 with an effective amount of a compound described herein, such as a compound of Formula I, I-A, I-B, I-C, II, or other compounds in section I, to inhibit the activity of said MALT1. In certain embodiments, the compound is a compound of Formula Ia, Ib, Ic, Id or defined by one of the embodiments described above.

Another aspect of the invention provides for the use of a compound described herein (such as a compound of Formula I, I-A, I-B, I-C, II, or other compounds in Section I) in the manufacture of a medicament. In certain embodiments, the medicament is for treating a disease or condition described herein, such as an inflammatory disorder or an allergic disorder. In certain embodiments, the compound is a compound of Formula Ia, Ib, Ic, Id or defined by one of the embodiments described above.

Another aspect of the invention provides for the use of a compound described herein (such as a compound of Formula I, I-A, I-B, I-C, II, or other compounds in Section I) for treating a disease or condition, such as a disease or condition described herein. In certain embodiments, the compound is a compound of Formula Ia, Ib, Ic, Id or defined by one of the embodiments described above.

In certain embodiments, the subject is a human. In certain embodiments, the subject is an adult human. In certain embodiments, the subject is a pediatric human. In certain embodiments, the subject is a geriatric human.

### Exemplary Diseases or Conditions

Exemplary diseases or conditions mediated by MALT1 include proliferative disorders (e.g., cancer, neoplasia), inflammatory disorders (e.g., chronic inflammatory disorder, acute inflammatory disorder, auto-inflammatory disorder), autoimmune disorders, fibrotic disorders, metabolic disorders, cardiovascular disorders, cerebrovascular disorders, and myeloid cell-driven hyper-inflammatory responses in COVID-19 infections.

In certain embodiments, the disease or condition mediated by MALT1 is a proliferative disorder. In certain embodiments, the disease or condition mediated by MALT1 is inflammatory disorder. In certain embodiments, the disease or condition mediated by MALT1 is an autoimmune disorder. In certain embodiments, the disease or condition mediated by MALT1 is a fibrotic disorder. In certain embodiments, the disease or condition mediated by MALT1 is a metabolic disorder. In certain embodiments, the disease or condition mediated by MALT1 is a cardiovascular disorder. In certain embodiments, the disease or condition mediated by MALT1 is a cerebrovascular disorder. In certain embodiments, the disease or condition mediated by MALT1 is a myeloid cell-driven hyper-inflammatory response in a COVID-19 infection.

In certain embodiments, the disease or condition mediated by MALT1 is cancer.

In certain embodiments, the cancer is selected from is non-small cell lung cancer (NSCLC), small cell lung cancer, colorectal cancer, rectal cancer, and pancreatic cancer. In certain embodiments, the cancer is selected from non-small cell lung cancer (NSCLC), pancreatic cancer, and colorectal cancer. In certain embodiments, the cancer is selected from non-small cell lung cancer (NSCLC) and pancreatic cancer.

In certain embodiments, the cancer is a solid tumor. In certain embodiments, the cancer is a melanoma, carcinoma, or blastoma. In certain embodiments, the cancer is a melanoma. In certain embodiments, the cancer is a carcinoma. In certain embodiments, the cancer is an adenocarcinoma. In certain embodiments, the cancer is a blastoma.

In certain embodiments, the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia. In certain embodiments, the cancer is lung cancer. In certain embodiments, the cancer is pancreatic cancer. In certain embodiments, the cancer is colorectal cancer. In certain embodiments, the cancer is breast cancer. In certain embodiments, the cancer is cervical cancer. In certain embodiments, the cancer is prostate cancer. In certain embodiments, the cancer is gastric cancer. In certain embodiments, the cancer is skin cancer. In certain embodiments, the cancer is liver cancer. In certain embodiments, the cancer is bile duct cancer. In certain embodiments, the cancer is nervous system cancer.

In certain embodiments, the cancer is breast adenocarcinoma, lung adenocarcinoma, pancreatic adenocarcinoma, cervical adenocarcinoma, colorectal adenocarcinoma, prostate adenocarcinoma, gastric adenocarcinoma, melanoma, lung squamous cell carcinoma, hepatocellular carcinoma, cholangiocarcinoma, glioblastoma, or neuroblastoma. In certain embodiments, the cancer is breast adenocarcinoma. In certain embodiments, the cancer is lung adenocarcinoma. In certain embodiments, the cancer is pancreatic adenocarcinoma. In certain embodiments, the cancer is cervical adenocarcinoma. In certain embodiments, the cancer is prostate adenocarcinoma. In certain embodiments, the cancer is gastric adenocarcinoma.

In certain embodiments, the cancer is melanoma.

In certain embodiments, the cancer is lung squamous cell carcinoma, hepatocellular carcinoma, or cholangiocarcinoma. In certain embodiments, the cancer is lung squamous cell carcinoma. In certain embodiments, the cancer is hepatocellular carcinoma. In certain embodiments, the cancer is cholangiocarcinoma.

In certain embodiments, the cancer is glioblastoma or neuroblastoma. In certain embodiments, the cancer is glioblastoma. In certain embodiments, the cancer is neuroblastoma.

In certain embodiments, the cancer is lung cancer, pancreatic cancer, or colorectal cancer. In certain embodiments, the cancer is non-small cell lung cancer, pancreatic cancer, or colorectal cancer. In certain embodiments, the cancer is lung cancer. In certain embodiments, the cancer is non-small cell lung cancer.

In certain embodiments, the cancer is a lymphoma or a leukemia. In certain embodiments, the cancer is a B-cell lymphoma or chronic myelocytic leukemia.

In certain embodiments, the cancer is a leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, or a solid tumor such as a sarcoma or carcinoma (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In certain embodiments, the cancer is MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

In certain embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In certain embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In certain embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor.

In certain embodiments, the cancer is mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, fallopian tube cancer, papillary serous cystadenocarcinoma, uterine papillary serous carcinoma (UPSC), prostate cancer, testicular cancer, gallbladder cancer, hepatocholangiocarcinoma, soft tissue and bone synovial sarcoma, rhabdomyosarcoma, osteosarcoma, chondrosarcoma, Ewing sarcoma, anaplastic thyroid cancer, adrenocortical adenoma, pancreatic cancer, pancreatic ductal carcinoma, pancreatic adenocarcinoma, gastrointestinal/stomach (GIST) cancer, lymphoma, squamous cell carcinoma of the head and neck (SCCHN), salivary gland cancer, glioma, or brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; and medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In certain embodiments, the cancer is hepatoblastoma. In certain embodiments, the cancer is colon cancer. In certain embodiments, the cancer is rectal cancer. In certain embodiments, the cancer is ovarian cancer, or ovarian carcinoma. In certain embodiments, the cancer is ovarian epithelial cancer. In certain embodiments, the cancer is fallopian tube cancer. In certain embodiments, the cancer is papillary serous cystadenocarcinoma. In certain embodiments, the cancer is uterine papillary serous carcinoma (UPSC). In certain embodiments, the cancer is hepatocholangiocarcinoma. In certain embodiments, the cancer is soft tissue and bone synovial sarcoma. In certain embodiments, the cancer is rhabdomyosarcoma. In certain embodiments, the cancer is osteosarcoma. In certain embodiments, the cancer is anaplastic thyroid cancer. In certain embodiments, the cancer is adrenocortical carcinoma. In certain embodiments, the cancer is pancreatic cancer, or pancreatic ductal carcinoma. In certain embodiments, the cancer is pancreatic adenocarcinoma. In certain embodiments, the cancer is glioma. In certain embodiments, the cancer is malignant peripheral nerve sheath tumors (MPNST). In certain embodiments, the cancer is neurofibromatosis-1 associated MPNST. In certain embodiments, the cancer is Waldenstrom's macroglobulinemia. In certain embodiments, the cancer is medulloblastoma.

In certain embodiments, the cancer is a lymphoma. In certain embodiments, the cancer is a leukemia. In certain embodiments, the cancer is Hodgkin's lymphoma. In certain embodiments, the cancer is non-Hodgkin's lymphoma. In certain embodiments, the cancer is Burkitt's lymphoma. In certain embodiments, the cancer is diffuse large B-cell lymphoma (DLBCL). In certain embodiments, the cancer is MALT lymphoma. In certain embodiments, the cancer is germinal center B-cell-like diffuse large B-cell lymphoma (GCB-DLBCL) or primary mediastinal B-cell lymphoma (PMBL). In certain embodiments, the cancer is activated B-cell-like diffuse large B-cell lymphoma (ABC-DLBCL). In certain embodiments, the cancer is a hematological cancer.

In certain embodiments, the proliferative disease is a cancer associated with or dependent on a MALT1 fusion protein (e.g., API2-MALT1). In certain embodiments, the proliferative disease is a cancer associated with dependence on B-cell lymphoma 10 (Bcl10). In certain embodiments, the proliferative disease is a cancer associated with dependence on caspase recruitment domain-containing protein (CARD1). In certain embodiments, the proliferative disease is a cancer associated with dependence on NF-κB. In certain embodidments, the cancer is a hematological malignancy.

Additional exemplary cancers include but are not limited to acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast, triple negative breast cancer (TNBC)); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; vulvar cancer (e.g., Paget's disease of the vulva); Burkitt lymphoma; primary intraocular lymphoma; classic Hodgkin lymphora; biphenotypic acute leukemia; T cell lymphoma; nasal-type T cell lymphoma; enteropathy-type T-cell lymphoma; subcutaneous panniculitis-like T-cel lymphoma; blastic NK-cell lymphoma; T-cell prolymphocytic leukemia, and NK-cell leukemia.

In certain embodiments, the cancer is a hematological malignancy. Exemplary hematological malignancies include but are not limited to leukemia, such as acute lymphoblastic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)), acute non-lymphocytic leukemia (ANLL), acute promyelocytic leukemia (APL), and acute myelomonocytic leukemia (AMMoL); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt's lymphoma, Waldenstrom's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; multiple myeloma (MM); heavy chain disease (e.g., alpha chain disease, gamma chain disease, mu chain disease), polycythemia vera, Wilm's tumor, and Ewing's sarcoma.

In certain embodiments, said disease or condition mediated by MALT1 is a multiple myeloma. In certain embodiments, said disease or condition mediated by MALT1 is a leukemia (e.g., acute lymphocytic leukemia, acute and chronic myelogenous leukemia, chronic lymphocytic leukemia, acute lymphoblastic leukemia, chronic myelomonocytic leukemia, or promyelocytic leukemia).

In certain embodiments, said disease or condition mediated by MALT1 is a lymphoma (e.g., B-cell lymphoma, T-cell lymphoma, mantle cell lymphoma, diffuse large B cell lymphoma, hairy cell lymphoma, Burkitt's lymphoma, mast cell tumors, Hodgkin's disease or non-Hodgkin's disease). In certain embodiments, said disease or condition mediated by MALT1 is myelodysplastic syndrome. In certain embodiments, said disease or condition mediated by MALT1 is fibrosarcoma. In certain embodiments, said disease or condition mediated by MALT1 is rhabdomyosarcoma. In certain embodiments, said disease or condition mediated by MALT1 is astrocytoma. In certain embodiments, said disease or condition mediated by MALT1 is neuroblastoma. In certain embodiments, said disease or condition mediated by MALT1 is glioma and schwannomas. In certain embodiments, said disease or condition mediated by MALT1 is melanoma. In certain embodiments, said disease or condition mediated by MALT1 is seminoma. In certain embodiments, said disease or condition mediated by MALT1 is teratocarcinoma. In certain embodiments, said disease or condition mediated by MALT1 is osteosarcoma. In certain embodiments, said disease or condition mediated by MALT1 is xenoderma pigmentosum. In certain embodiments, said disease or condition mediated by MALT1 is keratoctanthoma. In certain embodiments, said disease or condition mediated by MALT1 is thyroid follicular cancer. In certain embodiments, said disease or condition mediated by MALT1 is Kaposi's sarcoma. In certain embodiments, said disease or condition mediated by MALT1 is melanoma. In certain embodiments, said disease or condition mediated by MALT1 is teratoma. In certain embodiments, said disease or condition mediated by MALT1 is rhabdomyosarcoma. In certain embodiments, said disease or condition mediated by MALT1 is a metastatic and bone disorder. In certain embodiments, said disease or condition mediated by MALT1 is cancer of the bone. In certain embodiments, said disease or condition mediated by MALT1 is cancer of the mouth/pharynx. In certain embodiments, said disease or condition mediated by MALT1 is cancer of the esophagus. In certain embodiments, said disease or condition mediated by MALT1 is cancer of the larynx. In certain embodiments, said disease or condition mediated by MALT1 is cancer of the stomach. In certain embodiments, said disease or condition mediated by MALT1 is cancer of the intestine. In certain embodiments, said disease or condition mediated by MALT1 is cancer of the colon. In certain embodiments, said disease or condition mediated by MALT1 is cancer of the rectum. In certain embodiments, said disease or condition mediated by MALT1 is cancer of the lung (e.g., non-small cell lung cancer or small cell lung cancer). In certain embodiments, said disease or condition mediated by MALT1 is cancer of the liver. In certain embodiments, said disease or condition mediated by MALT1 is cancer of the pancreas. In certain embodiments, said disease or condition mediated by MALT1 is cancer of the nerve. In certain embodiments, said disease or condition mediated by MALT1 is cancer of the brain (e.g., glioma or glioblastoma multiforme). In certain embodiments, said disease or condition mediated by MALT1 is cancer of the head and neck. In certain embodiments, said disease or condition mediated by MALT1 is cancer of the throat. In certain embodiments, said disease or condition mediated by MALT1 is cancer of the ovary. In certain embodiments, said disease or condition mediated by MALT1 is cancer of the uterus. In certain embodiments, said disease or condition mediated by MALT1 is cancer of the prostate. In certain embodiments, said disease or condition mediated by MALT1 is cancer of the testis. In certain embodiments, said disease or condition mediated by MALT1 is cancer of the bladder. In certain embodiments, said disease or condition mediated by MALT1 is cancer of the kidney. In certain embodiments, said disease or condition mediated by MALT1 is cancer of the breast. In certain embodiments, said disease or condition mediated by MALT1 is cancer of the gall bladder. In certain embodiments, said disease or condition mediated by MALT1 is cancer of the cervix. In certain embodiments, said disease or condition mediated by MALT1 is cancer of the thyroid. In certain embodiments, said disease or condition mediated by MALT1 is cancer of the prostate. In certain embodiments, said disease or condition mediated by MALT1 is cancer of the skin (e.g., skin squamous cell carcinoma). In certain embodiments, said disease or condition mediated by MALT1 is a solid tumor. In certain embodiments, said disease or condition mediated by MALT1 is gastric cancer. In certain embodiments, said disease or condition mediated by MALT1 is hepatocellular carcinoma. In certain embodiments, said disease or condition mediated by MALT1 is a peripheral nerve sheath tumor. In certain embodiments, said disease or condition mediated by MALT1 is pulmonary arterial hypertension.

In certain embodiments, the disease is a cancer associated with a viral infection. In certain embodiments, the disease is a cancer resulting from infection with an oncovirus. In certain embodiments, the oncovirus is hepatitis A, hepatitis B, hepatitis C, human T-lymphotropic virus (HTLV), human papillomavirus (HPV), Kaposi's sarcoma-associated herpesvirus (HHV-8), Merkel cell polyomavirus, or Epstein-Barr virus (EBV). In certain embodiments, the disease is human T-lymphotropic virus. In certain embodiments, the disease is Kaposi's sarcoma-associated herpesvirus. In certain embodiments, the disease is Epstein-Barr virus. Leukemias and lymphomas which may be associated with an oncoviral include: for HTLV, adult T-cell leukemia; for HHV-8, Castleman's disease and primary effusion lymphoma; and for EBV, Burkitt's lymphoma, Hogdkin's lymphoma, and post-transplant lymphoproliferative disease.

In certain embodiments, said disease or condition mediated by MALT1 is an inflammatory disorder or allergic disorder. In certain embodiments, said disease or condition mediated by MALT1 is an inflammatory disorder, such as autoimmune disorders, chronic inflammatory disorders, acute inflammatory disorders, auto-inflammatory disorders, fibrotic disorders, metabolic disorders, neoplasias, cardiovascular or cerebrovascular disorders, and myeloid cell-driven hyper-inflammatory response in COVID-19 infections. In certain embodiments, said disease or condition mediated by MALT1 is an allergic disorder, such as asthma and allergic rhinitis.

In certain embodiments, said disease or condition mediated by MALT1 is a disease or disorder of tissues and systemic disease [e.g., systemic lupus erythematosus (SLE); immune thrombocytopenic purpura (ITP); autoimmune hemolytic anemia (AHA); autoimmune neutropenia (AIN); Evans syndrome; proliferative and hyperproliferative diseases, such as cancer, atherosclerosis, rheumatoid arthritis, psoriasis, idiopathic pulmonary fibrosis, scleroderma, cirrhosis of the liver; and Acquired Immunodeficiency Syndrome (AIDS)]. In certain embodiments, said disease or condition mediated by MALT1 is an immunologically-mediated disease, such as allograft rejection (e.g., rejection of transplanted organs or tissues). In certain embodiments, said disease or condition mediated by MALT1 is a tissue injury (e.g., associated with organ transplant or revascularization procedures). In certain embodiments, said disease or condition mediated by MALT1 is a disease or disorder of the respiratory tract (e.g., asthma). In certain embodiments, said disease or condition mediated by MALT1 is allergic rhinitis. In certain embodiments, said disease or condition mediated by MALT1 is a disease or disorder of the bone and joints (e.g., arthritis, rheumatoid arthritis). In certain embodiments, said disease or condition mediated by MALT1 is a disease or disorder of the skin. In certain embodiments, said disease or condition mediated by MALT1 is a disease or disorder of the gastrointestinal tract.

In certain embodiments, said disease or condition mediated by MALT1 is a reversible obstructive airways disease, such as asthma (e.g., bronchial asthma, allergic asthma, intrinsic asthma, extrinsic asthma, and dust asthma). In certain embodiments, said disease or condition mediated by MALT1 is chronic or inveterate asthma (e.g., late asthma airways hyper-responsiveness). In certain embodiments, said disease or condition mediated by MALT1 is bronchitis. In certain embodiments, said disease or condition mediated by MALT1 is a condition characterized by an inflammation of the nasal mucus membrane. In certain embodiments, said disease or condition mediated by MALT1 is acute rhinitis. In certain embodiments, said disease or condition mediated by MALT1 is allergic rhinitis. In certain embodiments, said disease or condition mediated by MALT1 is atrophic rhinitis. In certain embodiments, said disease or condition mediated by MALT1 is chronic rhinitis (e.g., rhinitis caseosa, hypertrophic rhinitis, rhinitis purulenta, rhinitis sicca, and rhinitis medicamentosa). In certain embodiments, said disease or condition mediated by MALT1 is membranous rhinitis (e.g., croupous rhinitis, fibrinous rhinitis, pseudomembranous rhinitis, and scrofoulous rhinitis). In certain embodiments, said disease or condition mediated by MALT1 is seasonal rhinitis [e.g., rhinitis nervosa (hay fever), vasomotor rhinitis, sarcoidosis, farmer's lung, and related diseases, such as fibroid lung and idiopathic interstitial pneumonia].

In certain embodiments, said disease or condition mediated by MALT1 includes pannus formation. In certain embodiments, said disease or condition mediated by MALT1 does not include pannus formation. In certain embodiments, said disease or condition mediated by MALT1 is rheumatoid arthritis. In certain embodiments, said disease or condition mediated by MALT1 is seronegative spondyloarthropathis (e.g., ankylosing spondylitis, psoriatic arthritis, and Reiter's disease). In certain embodiments, said disease or condition mediated by MALT1 is Behcet's disease. In certain embodiments, said disease or condition mediated by MALT1 is Sjogren's syndrome. In certain embodiments, said disease or condition mediated by MALT1 is systemic sclerosis.

In certain embodiments, said disease or condition mediated by MALT1 is psoriasis. In certain embodiments, said disease or condition mediated by MALT1 is systemic sclerosis. In certain embodiments, said disease or condition mediated by MALT1 is atopical dermatitis. In certain embodiments, said disease or condition mediated by MALT1 is contact dermatitis. In certain embodiments, said disease or condition mediated by MALT1 is eczematous dermatitis. In certain embodiments, said disease or condition mediated by MALT1 is seborrhoetic dermatitis. In certain embodiments, said disease or condition mediated by MALT1 is Lichen planus. In certain embodiments, said disease or condition mediated by MALT1 is Pemphigus. In certain embodiments, said disease or condition mediated by MALT1 is bullous Pemphigus. In certain embodiments, said disease or condition mediated by MALT1 is epidermolysis bullosa. In certain embodiments, said disease or condition mediated by MALT1 is urticaria. In certain embodiments, said disease or condition mediated by MALT1 is angiodermas. In certain embodiments, said disease or condition mediated by MALT1 is vasculitides. In certain embodiments, said disease or condition mediated by MALT1 is erythemas. In certain embodiments, said disease or condition mediated by MALT1 is cutaneous eosinophilias. In certain embodiments, said disease or condition mediated by MALT1 is uveitis. In certain embodiments, said disease or condition mediated by MALT1 is Alopecia. In certain embodiments, said disease or condition mediated by MALT1 is areata. In certain embodiments, said disease or condition mediated by MALT1 is vernal conjunctivitis.

In certain embodiments, said disease or condition mediated by MALT1 is Coeliac disease. In certain embodiments, said disease or condition mediated by MALT1 is proctitis. In certain embodiments, said disease or condition mediated by MALT1 is eosinophilic gastroenteritis. In certain embodiments, said disease or condition mediated by MALT1 is mastocytosis. In certain embodiments, said disease or condition mediated by MALT1 is pancreatitis. In certain embodiments, said disease or condition mediated by MALT1 is Crohn's disease. In certain embodiments, said disease or condition mediated by MALT1 is ulcerative colitis. In certain embodiments, said disease or condition mediated by MALT1 is a food-related allergy having effects remote from the gut (e.g., migraine, rhinitis, and eczema).

In certain embodiments, said disease or condition mediated by MALT1 is multiple sclerosis. In certain embodiments, said disease or condition mediated by MALT1 is artherosclerosis. In certain embodiments, said disease or condition mediated by MALT1 is acquired immunodeficiency syndrome (AIDS). In certain embodiments, said disease or condition mediated by MALT1 is lupus. In certain embodiments, said disease or condition mediated by MALT1 is lupus erythematosus. In certain embodiments, said disease or condition mediated by MALT1 is systemic lupus erythematosus. In certain embodiments, said disease or condition mediated by MALT1 is Hashimnoto's thyroiditis. In certain embodiments, said disease or condition mediated by MALT1 is myasthenia gravis. In certain embodiments, said disease or condition mediated by MALT1 is type I diabetes. In certain embodiments, said disease or condition mediated by MALT1 is nephrotic syndrome. In certain embodiments, said disease or condition mediated by MALT1 is eosinophilia fasciitis. In certain embodiments, said disease or condition mediated by MALT1 is hyper IgE syndrome. In certain embodiments, said disease or condition mediated by MALT1 is lepromatous leprosy. In certain embodiments, said disease or condition mediated by MALT1 is sezary syndrome. In certain embodiments, said disease or condition mediated by MALT1 is idiopathic thrombocytopenia purpura. In certain embodiments, said disease or condition mediated by MALT1 is restenosis following angioplasty. In certain embodiments, said disease or condition mediated by MALT1 is a tumor (e.g., leukemia, lymphomas). In certain embodiments, said disease or condition mediated by MALT1 is artherosclerosis.

In certain embodiments, said disease or condition mediated by MALT1 is acute chronic allograft rejection (e.g., following transplantation of kidney, heart, liver, lung, bone marrow, skin, or cornea). In certain embodiments, said disease or condition mediated by MALT1 is chronic allograft rejection (e.g., following transplantation of kidney, heart, liver, lung, bone marrow, skin, or cornea). In certain embodiments, said disease or condition mediated by MALT1 is chronic graft-versus-host disease.

In certain embodiments, said disease or condition mediated by MALT1 is an acute inflammatory disorder. In certain embodiments, said disease or condition mediated by MALT1 is an auto-inflammatory disorder. In certain embodiments, said disease or condition mediated by MALT1 is a fibrotic disorder. In certain embodiments, said disease or condition mediated by MALT1 is a metabolic disorder. In certain embodiments, said disease or condition mediated by MALT1 is a neoplasia. In certain embodiments, said disease or condition mediated by MALT1 is a cardiovascular or cerebrovascular disorder. In certain embodiments, said disease or condition mediated by MALT1 is a myeloid cell-driven hyper-inflammatory response in COVID-19 infections.

In certain embodiments, said disease or condition mediated by MALT1 is an autoimmune disorder. In certain embodiments, said disease or condition mediated by MALT1 is a chronic inflammatory disorder. In certain embodiments, said disease or condition mediated by MALT1 is an acute inflammatory disorder. In certain embodiments, said disease or condition mediated by MALT1 is an auto-inflammatory disorder. In certain embodiments, said disease or condition mediated by MALT1 is a combination of one, two, or all three of a chronic inflammatory disorder, an acute inflammatory disorder, and an auto-inflammatory disorder.

In certain embodiments, said disease or condition mediated by MALT1 is an inflammatory bowel disease (e.g., ulcerative colitis or Crohn's disease). In certain embodiments, said disease or condition mediated by MALT1 is multiple sclerosis. In certain embodiments, said disease or condition mediated by MALT1 is psoriasis. In certain embodiments, said disease or condition mediated by MALT1 is arthritis. In certain embodiments, said disease or condition mediated by MALT1 is rheumatoid arthritis. In certain embodiments, said disease or condition mediated by MALT1 is osteoarthritis. In certain embodiments, said disease or condition mediated by MALT1 is juvenile arthritis. In certain embodiments, said disease or condition mediated by MALT1 is psoriatic arthritis. In certain embodiments, said disease or condition mediated by MALT1 is reactive arthritis. In certain embodiments, said disease or condition mediated by MALT1 is ankylosing spondylitis. In certain embodiments, said disease or condition mediated by MALT1 is cryopyrin-associated periodic syndromes. In certain embodiments, said disease or condition mediated by MALT1 is Muckle-Wells syndrome. In certain embodiments, said disease or condition mediated by MALT1 is familial cold auto-inflammatory syndrome. In certain embodiments, said disease or condition mediated by MALT1 is neonatal-onset multisystem inflammatory disease. In certain embodiments, said disease or condition mediated by MALT1 is TNF receptor-associated periodic syndrome. In certain embodiments, said disease or condition mediated by MALT1 is acute and chronic pancreatitis. In certain embodiments, said disease or condition mediated by MALT1 is atherosclerosis. In certain embodiments, said disease or condition mediated by MALT1 is gout. In certain embodiments, said disease or condition mediated by MALT1 is a fibrotic disorder (e.g., hepatic fibrosis or idiopathic pulmonary fibrosis). In certain embodiments, said disease or condition mediated by MALT1 is nephropathy. In certain embodiments, said disease or condition mediated by MALT1 is sarcoidosis. In certain embodiments, said disease or condition mediated by MALT1 is scleroderma. In certain embodiments, said disease or condition mediated by MALT1 is anaphylaxis. In certain embodiments, said disease or condition mediated by MALT1 is diabetes (e.g., diabetes mellitus type 1 or diabetes mellitus type 2). In certain embodiments, said disease or condition mediated by MALT1 is diabetic retinopathy. In certain embodiments, said disease or condition mediated by MALT1 is Still's disease. In certain embodiments, said disease or condition mediated by MALT1 is vasculitis. In certain embodiments, said disease or condition mediated by MALT1 is sarcoidosis. In certain embodiments, said disease or condition mediated by MALT1 is pulmonary inflammation. In certain embodiments, said disease or condition mediated by MALT1 is respiratory failure. In certain embodiments, said disease or condition mediated by MALT1 is acute respiratory distress syndrome. In certain embodiments, said disease or condition mediated by MALT1 is chronic eosinophilic pneumonia. In certain embodiments, said disease or condition mediated by MALT1 is wet and dry age-related macular degeneration. In certain embodiments, said disease or condition mediated by MALT1 is autoimmune hemolytic syndromes. In certain embodiments, said disease or condition mediated by MALT1 is autoimmune and inflammatory hepatitis. In certain embodiments, said disease or condition mediated by MALT1 is autoimmune neuropathy. In certain embodiments, said disease or condition mediated by MALT1 is autoimmune ovarian failure. In certain embodiments, said disease or condition mediated by MALT1 is autoimmune orchitis. In certain embodiments, said disease or condition mediated by MALT1 is autoimmune thrombocytopenia. In certain embodiments, said disease or condition mediated by MALT1 is silicone implant-associated autoimmune disease. In certain embodiments, said disease or condition mediated by MALT1 is Sjogren's syndrome. In certain embodiments, said disease or condition mediated by MALT1 is familial Mediterranean fever. In certain embodiments, said disease or condition mediated by MALT1 is systemic lupus erythematosus. In certain embodiments, said disease or condition mediated by MALT1 is vasculitis syndromes (e.g., t. emporal, Takayasu's and giant cell arteritis, Behcet's disease or Wegener's granulomatosis). In certain embodiments, said disease or condition mediated by MALT1 is vitiligo. In certain embodiments, said disease or condition mediated by MALT1 is secondary hematologic manifestation of autoimmune diseases (e.g., anemias). In certain embodiments, said disease or condition mediated by MALT1 is drug-induced autoimmunity. In certain embodiments, said disease or condition mediated by MALT1 is Hashimoto's thyroiditis. In certain embodiments, said disease or condition mediated by MALT1 is hypophysitis. In certain embodiments, said disease or condition mediated by MALT1 is idiopathic thrombocytic pupura. In certain embodiments, said disease or condition mediated by MALT1 is metal-induced autoimmunity. In certain embodiments, said disease or condition mediated by MALT1 is myasthenia gravis. In certain embodiments, said disease or condition mediated by MALT1 is pemphigus. In certain embodiments, said disease or condition mediated by MALT1 is autoimmune deafness (e.g., Meniere's disease). In certain embodiments, said disease or condition mediated by MALT1 is Goodpasture's syndrome. In certain embodiments, said disease or condition mediated by MALT1 is Graves' disease. In certain embodiments, said disease or condition mediated by MALT1 is an HW-related autoimmune syndromes. In certain embodiments, said disease or condition mediated by MALT1 is Gullain-Barre disease. In certain embodiments, said disease or condition mediated by MALT1 is Addison's disease. In certain embodiments, said disease or condition mediated by MALT1 is anti-phospholipid syndrome. In certain embodiments, said disease or condition mediated by MALT1 is asthma. In certain embodiments, said disease or condition mediated by MALT1 is atopic dermatitis. In certain embodiments, said disease or condition mediated by MALT1 is Celiac disease. In certain embodiments, said disease or condition mediated by MALT1 is Cushing's syndrome. In certain embodiments, said disease or condition mediated by MALT1 is dermatomyositis. In certain embodiments, said disease or condition mediated by MALT1 is idiopathic adrenal atrophy. In certain embodiments, said disease or condition mediated by MALT1 is idiopathic thrombocytopenia. In certain embodiments, said disease or condition mediated by MALT1 is Kawasaki syndrome. In certain embodiments, said disease or condition mediated by MALT1 is Lambert-Eaton Syndrome. In certain embodiments, said disease or condition mediated by MALT1 is pernicious anemia. In certain embodiments, said disease or condition mediated by MALT1 is pollinosis. In certain embodiments, said disease or condition mediated by MALT1 is polyarteritis nodosa. In certain embodiments, said disease or condition mediated by MALT1 is primary biliary cirrhosis. In certain embodiments, said disease or condition mediated by MALT1 is primary sclerosing cholangitis. In certain embodiments, said disease or condition mediated by MALT1 is Raynaud's disease. In certain embodiments, said disease or condition mediated by MALT1 is Raynaud's phenomenon. In certain embodiments, said disease or condition mediated by MALT1 is Reiter's Syndrome. In certain embodiments, said disease or condition mediated by MALT1 is relapsing polychondritis. In certain embodiments, said disease or condition mediated by MALT1 is Schmidt's syndrome. In certain embodiments, said disease or condition mediated by MALT1 is thyrotoxidosis. In certain embodiments, said disease or condition mediated by MALT1 is sepsis. In certain embodiments, said disease or condition mediated by MALT1 is septic shock. In certain embodiments, said disease or condition mediated by MALT1 is endotoxic shock. In certain embodiments, said disease or condition mediated by MALT1 is exotoxin-induced toxic shock. In certain embodiments, said disease or condition mediated by MALT1 is gram negative sepsis. In certain embodiments, said disease or condition mediated by MALT1 is toxic shock syndrome. In certain embodiments, said disease or condition mediated by MALT1 is glomerulonephritis. In certain embodiments, said disease or condition mediated by MALT1 is peritonitis. In certain embodiments, said disease or condition mediated by MALT1 is interstitial cystitis. In certain embodiments, said disease or condition mediated by MALT1 is hyperoxia-induced inflammations. In certain embodiments, said disease or condition mediated by MALT1 is chronic obstructive pulmonary disease (COPD). In certain embodiments, said disease or condition mediated by MALT1 is emphysema. In certain embodiments, said disease or condition mediated by MALT1 is nasal inflammation. In certain embodiments, said disease or condition mediated by MALT1 is vasculitis. In certain embodiments, said disease or condition mediated by MALT1 is graft vs. host reaction (e.g., graft vs. host disease). In certain embodiments, said disease or condition mediated by MALT1 is allograft rejections (e.g., acute allograft rejection or chronic allograft rejection). In certain embodiments, said disease or condition mediated by MALT1 is early transplantation rejection (e.g., acute allograft rejection). In certain embodiments, said disease or condition mediated by MALT1 is reperfusion injury. In certain embodiments, said disease or condition mediated by MALT1 is pain (e.g., acute pain, chronic pain, neuropathic pain, or fibromyalgia). In certain embodiments, said disease or condition mediated by MALT1 is a chronic infection. In certain embodiments, said disease or condition mediated by MALT1 is meningitis. In certain embodiments, said disease or condition mediated by MALT1 is encephalitis. In certain embodiments, said disease or condition mediated by MALT1 is myocarditis. In certain embodiments, said disease or condition mediated by MALT1 is gingivitis. In certain embodiments, said disease or condition mediated by MALT1 is post-surgical trauma. In certain embodiments, said disease or condition mediated by MALT1 is tissue injury. In certain embodiments, said disease or condition mediated by MALT1 is traumatic brain injury. In certain embodiments, said disease or condition mediated by MALT1 is enterocolitis. In certain embodiments, said disease or condition mediated by MALT1 is sinusitis. In certain embodiments, said disease or condition mediated by MALT1 is uveitis. In certain embodiments, said disease or condition mediated by MALT1 is ocular inflammation. In certain embodiments, said disease or condition mediated by MALT1 is optic neuritis. In certain embodiments, said disease or condition mediated by MALT1 is gastric ulcers. In certain embodiments, said disease or condition mediated by MALT1 is esophagitis. In certain embodiments, said disease or condition mediated by MALT1 is peritonitis. In certain embodiments, said disease or condition mediated by MALT1 is periodontitis. In certain embodiments, said disease or condition mediated by MALT1 is dermatomyositis. In certain embodiments, said disease or condition mediated by MALT1 is gastritis. In certain embodiments, said disease or condition mediated by MALT1 is myositis. In certain embodiments, said disease or condition mediated by MALT1 is polymyalgia. In certain embodiments, said disease or condition mediated by MALT1 is pneumonia. In certain embodiments, said disease or condition mediated by MALT1 is bronchitis. In certain embodiments, the disease or condition mediated by MALT1 is endometriosis. In certain embodiments, the disease or condition mediated by MALT1 is necrotizing vasculitis. In certain embodiments, the disease or condition mediated by MALT1 is lymphadenitis. In certain embodiments, the disease or condition mediated by MALT1 is periarteritis nodosa. In certain embodiments, the disease or condition mediated by MALT1 is anti-phospholipid antibody syndrome. In certain embodiments, the disease or condition mediated by MALT1 is pemphigus vulgaris. In certain embodiments, the disease or condition mediated by MALT1 is Lyme disease. In certain embodiments, the disease or condition mediated by MALT1 is cardiomyopathy. In certain embodiments, the disease or condition mediated by MALT1 isrheumatic fever. In certain embodiments, the disease or condition mediated by MALT1 is a blistering disorder. In certain embodiments, the disease or condition mediated by MALT1 is an antibody-mediated vasculitis syndrome. In certain embodiments, the disease or condition mediated by MALT1 is an immune-complex vasculitide. In certain embodiments, the disease or condition mediated by MALT1 i, oedema. In certain embodiments, the disease or condition mediated by MALT1 is embolism. In certain embodiments, the disease or condition mediated by MALT1 is fibrosis. In certain embodiments, the disease or condition mediated by MALT1 is silicosis. In certain embodiments, the disease or condition mediated by MALT1 is BENTA disease. In certain embodiments, the disease or condition mediated by MALT1 is berylliosis.

In certain embodiments, said disease or condition mediated by MALT1 is systemic sclerosis/scleroderma. In certain embodiments, said disease or condition mediated by MALT1 is lupus nephritis. In certain embodiments, said disease or condition mediated by MALT1 is connective tissue disease. In certain embodiments, said disease or condition mediated by MALT1 is wound healing. In certain embodiments, said disease or condition mediated by MALT1 is surgical scarring. In certain embodiments, said disease or condition mediated by MALT1 is spinal cord injury. In certain embodiments, said disease or condition mediated by MALT1 is CNS scarring. In certain embodiments, said disease or condition mediated by MALT1 is acute lung injury. In certain embodiments, said disease or condition mediated by MALT1 is pulmonary fibrosis (e.g., idiopathic pulmonary fibrosis or cystic fibrosis). In certain embodiments, said disease or condition mediated by MALT1 is chronic obstructive pulmonary disease. In certain embodiments, said disease or condition mediated by MALT1 is adult respiratory distress syndrome. In certain embodiments, said disease or condition mediated by MALT1 is acute lung injury. In certain embodiments, said disease or condition mediated by MALT1 is drug-induced lung injury. In certain embodiments, said disease or condition mediated by MALT1 is glomerulonephritis. In certain embodiments, said disease or condition mediated by MALT1 is chronic kidney disease (e.g., diabetic nephropathy). In certain embodiments, said disease or condition mediated by MALT1 is hypertension-induced nephropathy. In certain embodiments, said disease or condition mediated by MALT1 is alimentary track or gastrointestinal fibrosis. In certain embodiments, said disease or condition mediated by MALT1 is renal fibrosis. In certain embodiments, said disease or condition mediated by MALT1 is hepatic or biliary fibrosis. In certain embodiments, said disease or condition mediated by MALT1 is liver fibrosis (e.g., nonalcoholic steatohepatitis, hepatitis C, or hepatocellular carcinoma). In certain embodiments, said disease or condition mediated by MALT1 is cirrhosis (e.g., primary biliary cirrhosis or cirrhosis due to fatty liver disease, such as alcoholic and nonalcoholic steatosis). In certain embodiments, said disease or condition mediated by MALT1 is radiation-induced fibrosis (e.g., head and neck, gastrointestinal or pulmonary). In certain embodiments, said disease or condition mediated by MALT1 is primary sclerosing cholangitis. In certain embodiments, said disease or condition mediated by MALT1 is restenosis. In certain embodiments, said disease or condition mediated by MALT1 is cardiac fibrosis (e.g., endomyocardial fibrosis or atrial fibrosis). In certain embodiments, said disease or condition mediated by MALT1 is opthalmic scarring. In certain embodiments, said disease or condition mediated by MALT1 is fibrosclerosis. In certain embodiments, said disease or condition mediated by MALT1 is a fibrotic cancer. In certain embodiments, said disease or condition mediated by MALT1 is fibroids. In certain embodiments, said disease or condition mediated by MALT1 is fibroma. In certain embodiments, said disease or condition mediated by MALT1 is a fibroadenoma. In certain embodiments, said disease or condition mediated by MALT1 is a fibrosarcoma. In certain embodiments, said disease or condition mediated by MALT1 is transplant arteriopathy. In certain embodiments, said disease or condition mediated by MALT1 is keloid. In certain embodiments, said disease or condition mediated by MALT1 is mediastinal fibrosis. In certain embodiments, said disease or condition mediated by MALT1 is myelofibrosis. In certain embodiments, said disease or condition mediated by MALT1 is retroperitoneal fibrosis. In certain embodiments, said disease or condition mediated by MALT1 is progressive massive fibrosis. In certain embodiments, said disease or condition mediated by MALT1 is nephrogenic systemic fibrosis.

In certain embodiments, said disease or condition mediated by MALT1 is obesity. In certain embodiments, said disease or condition mediated by MALT1 is steroid-resistance. In certain embodiments, said disease or condition mediated by MALT1 is glucose intolerance. In certain embodiments, said disease or condition mediated by MALT1 is metabolic syndrome.

In certain embodiments, said disease or condition mediated by MALT1 is atherosclerosis. In certain embodiments, said disease or condition mediated by MALT1 is restenosis of an atherosclerotic coronary artery. In certain embodiments, said disease or condition mediated by MALT1 is acute coronary syndrome. In certain embodiments, said disease or condition mediated by MALT1 is myocardial infarction. In certain embodiments, said disease or condition mediated by MALT1 is cardiac-allograft vasculopathy. In certain embodiments, said disease or condition mediated by MALT1 is stroke. In certain embodiments, said disease or condition mediated by MALT1 is a central nervous system disorder with an inflammatory or apoptotic component. In certain embodiments, said disease or condition mediated by MALT1 is Alzheimer's disease. In certain embodiments, said disease or condition mediated by MALT1 is Parkinson's disease. In certain embodiments, said disease or condition mediated by MALT1 is Huntington's disease. In certain embodiments, said disease or condition mediated by MALT1 is amyotrophic lateral sclerosis. In certain embodiments, said disease or condition mediated by MALT1 is spinal cord injury. In certain embodiments, said disease or condition mediated by MALT1 is neuronal ischemia. In certain embodiments, said disease or condition mediated by MALT1 is peripheral neuropathy.

In certain embodiments, said disease or condition mediated by MALT1 is a disease or disorder associated with a coronavirus (e.g., SARS-CoV-2). In certain embodiments, said coronavirus is SARS-CoV-2. In certain embodiments, the disease or disorder associated with SARS-CoV-2 is COVID-19.

In certain embodiments, the disease or condition mediated by MALT1 is a rheumatic disease. In certain embodiments, the disease or condition mediated by MALT1 is an inflammatory arthropathy. In certain embodiments, the disease or condition mediated by MALT1 is rheumatoid arthritis, juvenile arthritis, Still's disease, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, ankylosing spondylitis, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile Idiopathic Arthritis (JIA), juvenile psoriatic arthritis, juvenile rheumatoid arthritis, systemic onset juvenile rheumatoid arthritis, giant cell arteritis, secondary osteoarthritis from an inflammatory disease.

In certain embodiments, the disease or condition mediated by MALT1 is a connective tissue disease. In certain embodiments, the disease or condition mediated by MALT1 is lupus, systemic lupus erythematosus, juvenile systemic lupus erythematosus, nephritis, Sjögren's syndrome, scleroderma (systemic sclerosis), Raynaud's phenomenonjuvenile scleroderma, polymyositis, dermatomyositis, polymyositis-dermatomyositis, polymyalgia rheumatica, a mixed connective tissue disease, sarcoidosis, fibromyalgia, vasculitis microscopic polyangiitis, vasculitis, eosinophilic granulomatosis with polyangiitis (formerly known as Churg-Strauss Syndrome), granulomatosis with polyangiitis (formerly known as Wegener's granulomatosis), polyarteritis nodosa, Henoch-Schonlein purpura, idiopathic thrombocytopenic thrombotic purpura, juvenile vasculitis, polyarteritis nodossa (also known as panarteritis nodosa, periarteritis nodosa Kussmaul disease, Kussmaul-Maier disease or PAN), serum sickness, myasthenia gravis, Takayasu's arteritis, Behget's syndrome, Kawasaki's disease (mucocutaneous lymph node syndrome), Buerger's disease (thromboangiitis obliterans), Vogt-Koyanagi-Harada syndrome, Addison's disease, Hashimoto's thyroiditis, primary biliary sclerosis, autoimmune hepatitis, chronic aggressive hepatitis, nonalcoholic hepatic steatosis, sclerosing cholangitis, membranous glomerulopathy, polymyositis, myositis, atherosclerosis, autoimmune hemolytic anemia, autoimmune orchitis, Goodpasture's disease,

In certain embodiments, the disease or condition mediated by MALT1 is a neurodegenerative disease or neuroinflammatory disease. In certain embodiments, the disease or condition mediated by MALT1 is multiple sclerosis, amyotropic lateral sclerosis, Guillain-Barre disease, autoimmune encephalomyelitis, Alzheimer's disease, major depressive disorder, traumatic brain injury, epilepsy, Parkinson's disease, or bipolar disorder.

In certain embodiments, the disease or condition mediated by MALT1 is an inflammatory bowel disease. In certain embodiments, the disease or condition mediated by MALT1 is Crohn's disease, ulcerative colitis, Celiac Sprue, Celiac disease, proctitis, eosinophilic gastroenteritis, autoimmune atrophic gastritis of pernicious anemia, or mastocytosis.

In certain embodiments, the disease or condition mediated by MALT1 is a skin autoimmune disorder. In certain embodiments, the disease or condition mediated by MALT1 is psoriasis. In certain embodiments, the disease or condition mediated by MALT1 is eczema. In certain embodiments, the disease or condition mediated by MALT1 is plaque psoriasis, Guttate psoriasis, psoriatic epidermal hyperplasia, inverse psoriasis, pustular psoriasis, erythrodermic psoriasis, atopic dermatitis, eczema dermatitis, dermatitis, rosacea, pruritus, alopecia areata, vitiligo, epidermal hyperplasia, juvenile dermatomyositis, dermatomyositis, or hidradenitis suppurativa.

In certain embodiments, the disease or condition mediated by MALT1 is an organ or cell transplant rejection. In certain embodiments, the disease or condition mediated by MALT1 is graft-versus-host disease. In certain embodiments, the disease or condition mediated by MALT1 is chronic graft-versus-host disease, acute graft-versus-host disease, or organ or cell transplant rejection such as bone marrow, cartilage, cornea, heart, intervertebral disc, islet, kidney, limb, liver, lung, muscle, myoblast, nerve, pancreas, skin, small intestine, or trachea, or xeno transplantation.

In certain embodiments, the disease or condition mediated by MALT1 is an autoimmune disease of the eye. In certain embodiments, the disease or condition mediated by MALT1 is Graves' disease, noninfectious uveitis, dry eye syndrome, sympathetic ophthalmia, Cogan's syndrome, keratoconjunctivitis, vernal conjunctivitis, uveitis (e.g., uveitis associated with Behcet's disease and lens-induced uveitis), keratitis, herpetic keratitis, conical keratitis, corneal epithelial dystrophy, keratoleukoma, ocular premphigus, Mooren's ulcer, scleritis, keratoconjunctivitis sicca (dry eye), phlyctenule, iridocyclitis, sarcoidosis, endocrine ophthalmopathy, sympathetic ophthalmitis, allergic conjunctivitis, or ocular neovascularization

In certain embodiments, the disease or condition mediated by MALT1 is an ocular manifestation of an autoimmune disease.

In certain embodiments, the disease or condition mediated by MALT1 is a respiratory disease. In certain embodiments, the disease or condition mediated by MALT1 is asthma, chronic obstructive pulmonary disease, or acute respiratory disease.

In certain embodiments, the disease or condition mediated by MALT1 is diabetes. In certain embodiments, the disease or condition mediated by MALT1 is Type I diabetes mellitus, Type II diabetes mellitus, or juvenile onset diabetes.

### Additional Methods

Another aspect of the invention provides methods of inhibiting cell proliferation in a subject by administering to the subject a compound described herein (e.g., a compound of Formula I, I-A, I-B, I-C, II, or other compounds in section I), or inhibiting cell proliferation in a biological sample by contacting the biological sample with a compound described herein (e.g., a compound of Formula I, I-A, I-B, I-C, II, or other compounds in section I). In certain embodiments, the compound is a compound of Formula Ia, Ib, Ic, Id or defined by one of the embodiments described above. In certain embodiments, cell proliferation is inhibited for T-cells. In certain embodiments, cell proliferation is inhibited for B-cells. In certain embodiments, cell proliferation is inhibited for T-cells and B-cells.

Another aspect of the invention provides methods of inducing apoptosis of a cell in a subject by administering to the subject a compound described herein (e.g., a compound of Formula I, I-A, I-B, I-C, II, or other compounds in section I), or inducing apoptosis of a cell in a biological sample by contacting the biological sample with a compound described herein (e.g., a compound of Formula I, I-A, I-B, I-C, II, or other compounds in section I). In certain embodiments, the compound is a compound of Formula Ia, Ib, Ic, Id or defined by one of the embodiments described above. In certain embodiments, cell is a tumor cell. In certain embodiments, the cell is a lymphocyte. In certain embodiments, the cell is a T-cell. In certain embodiments, the cell is a B-cell.

Another aspect of the invention provides methods of inhibiting adhesion of a cell in a subject by administering to the subject a compound described herein (e.g., a compound of Formula I, I-A, I-B, I-C, II, or other compounds in section I), or inhibiting adhesion of a cell in a biological sample by contacting the biological sample with a compound described herein (e.g., a compound of Formula I, I-A, I-B, I-C, II, or other compounds in section I). In certain embodiments, the compound is a compound of Formula Ia, Ib, Ic, Id or defined by one of the embodiments described above. In certain embodiments, the cell is a tumor cell. In certain embodiments, the cell is a lymphocyte. In certain embodiments, the cell is a T-cell. In certain embodiments, the cell is a B-cell.

Another aspect of the invention provides methods of inhibiting activation of T-cells or B-cells in a subject by administering to the subject a compound described herein (e.g., a compound of Formula I, I-A, I-B, I-C, II, or other compounds in section I), or inhibiting activation of T-cells or B-cells in a biological sample by contacting the biological sample with a compound described herein (e.g., a compound of Formula I, I-A, I-B, I-C, II, or other compounds in section I). In certain embodiments, the compound is a compound of Formula Ia, Ib, Ic, Id or defined by one of the embodiments described above.

Another aspect of the invention provides methods of inhibiting the activity of mucosa-associated lymphoid tissue lymphoma translation protein 1 (MALT1) or a MALT1 fusion protein in a subject by administering to the subject a compound described herein (e.g., a compound of Formula I, I-A, I-B, I-C, II, or other compounds in section I), or inhibiting the activity of mucosa-associated lymphoid tissue lymphoma translation protein 1 (MALT1) or a MALT1 fusion protein in a biological sample by contacting the biological sample with a compound described herein (e.g., a compound of Formula I, I-A, I-B, I-C, II, or other compounds in section I). In certain embodiments, the compound is a compound of Formula Ia, Ib, Ic, Id or defined by one of the embodiments described above. In certain embodiments, the method inhibits the protease activity of MALT1. In certain embodiments, the method inhibits the protease activity of a MALT1 fusion protein (e.g., API2-MALT1). In certain embodiments, the method inhibits the protease activity of MALT1 or a MALT1 fusion protein for cleavage of a peptide substrate. In certain embodiments, the peptide substrate is A20, Bcl10, RelB, CYLD, NIK, regnase-1, roquin-1, roquin-2, LIMAla, or MALT1. The inhibitor may selectively inhibit the protease activity of MALT1 or a MALT1 fusion protein for cleavage of a first peptide substrate over protease activity for cleavage of a second peptide substrate. In certain embodiments, the first and/or second substrate is A20, Bcl10, RelB, CYLD, NIK, regnase-1, roquin-1, roquin-2, LIMA1α, or MALT1. In certain embodiments, the selectivity is between about 1.25 fold and about 5 fold. In certain embodiments, the selectivity is between about 5 fold and about 10 fold. In certain embodiments, the selectivity is between about 10 fold and about 25 fold. In certain embodiments, the selectivity is between about 25 fold and about 50 fold. In certain embodiments, the selectivity is between about 50 fold and about 100 fold. In certain embodiments, the selectivity is between about 100 fold and about 250 fold. In certain embodiments. In certain embodiments, the selectivity is between about 250 fold and about 500 fold. In certain embodiments, the selectivity is between about 500 fold and about 1000 fold. In certain embodiments, or at least about 1000 fold.

### III. COMBINATION THERAPY

Another aspect of the invention provides for combination therapy. Pyridinylsulfonamide compounds described herein (e.g., a compound of Formula I, I-A, I-B, I-C, II, or other compounds in Section I) or their pharmaceutically acceptable salts may be used in combination with additional therapeutic agents to treat diseases or conditions, such as an inflammatory disorder.

Accordingly, In certain embodiments, the present invention provides a method of treating a disclosed disease or condition comprising administering to a patient in need thereof an effective amount of a compound disclosed herein and co-administering simultaneously or sequentially an effective amount of one or more additional therapeutic agents, such as those described herein. In certain embodiments, the method includes co-administering one additional therapeutic agent. In certain embodiments, the method includes co-administering two additional therapeutic agents.

One or more other therapeutic agents may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another.

In certain embodiments, the compounds of the disclosure can be administered with one or more of a second therapeutic agent, sequentially or concurrently, either by the same route or by different routes of administration. When administered sequentially, the time between administrations is selected to benefit, among others, the therapeutic efficacy and/or safety of the combination treatment. In certain embodiments, the compound of the disclosure can be administered first followed by a second therapeutic agent, or alternatively, the second therapeutic agent administered first followed by the compound of the disclosure. In certain embodiments, the compound of the disclosure can be administered for the same duration as the second therapeutic agent, or alternatively, for a longer or shorter duration as the second therapeutic compound.

When administered concurrently, the compounds of the disclosure can be administered separately at the same time as the second therapeutic agent, by the same or different routes, or administered in a single composition by the same route. In certain embodiments, the compound of the disclosure is prepared as a first pharmaceutical composition, and the second therapeutic agent prepared as a second pharmaceutical composition, where the first pharmaceutical composition and the second pharmaceutical composition are administered simultaneously, sequentially, or separately. In certain embodiments, the amount and frequency of administration of the second therapeutic agent can used standard dosages and standard administration frequencies used for the particular therapeutic agent. See, e.g., Physicians' DeskReference, 70th Ed., PDR Network, 2015; incorporated herein by reference.

In certain embodiments, the additional therapeutic agent is a leukotriene inhibitor, non-steroidal anti-inflammatory drug (NSAID), steroid, tyrosine kinase inhibitor, receptor kinase inhibitor, modulator of nuclear receptor family of transcription factor, HSP90 inhibitor, adenosine receptor (A₂A) agonist, disease modifying antirheumatic drugs (DMARDS), phosphodiesterase (PDE) inhibitor, neutrophil elastase inhibitor, modulator of Axl kinase, an anti-cancer agent, anti-allergic agent, anti-nausea agent (or anti-emetic), pain reliever, cytoprotective agent, or a combination thereof. In certain embodiments, the additional therapeutic agent is an anti-cancer agent, an analgesic, an anti-inflammatory agent, or a combination thereof.

In certain embodiments, the second therapeutic agent is a bispecific antibody, such as a bispecific antibody that binds to a tumor-specific antigen. Exemplary bispecific antibodies include but are not limited to Blincyto (blinatumomab), Kimmtrak (tebentafusp), Tecvayli (teclistamab), Lunsumio (mosunetuzumab), Epkinly (epcoritamab), and Columvi (glofitamab).

In certain embodiments, the second therapeutic agent is a chimeric antigen receptor (CAR) T-cell therapy. Exemplary CAR T-cell therapies include but are not limited to ABECMA® (idecabtagene vicleucel), BREYANZI® (lisocabtagene maraleucel), CARVYKTI™ (ciltacabtagene autoleucel), KYMRIAH™ (tisagenlecleucel), TECARTUS™ (brexucabtagene autoleucel), and YESCARTA™ (axicabtagene ciloleucel).

In certain embodiments, the second therapeutic agent is a leukotriene inhibitor. Examples of leukotriene inhibitors considered for use in combination therapies of the invention include but are not limited to montelukast, zafirlukast, pranlukast, zileuton, or combinations thereof.

In certain embodiments, the second therapeutic agent is a an NSAID. Examples of NSAIDs considered for use in combination therapies of the invention include but are not limited to acetylsalicylic acid, diflunisal, salsalate, ibuprofen, dexibuprofen, naioxen, fenoprofen, ketoprofen, dexketoprofen, flurbiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, phenylbutazone, mefenamic acid, meclofenamic acid, flufenamic acid, tolfenamic acid, celecoxib, or combinations thereof.

In certain embodiments, the second therapeutic agent is a steroid. Examples of steroids considered for use in combination therapies of the invention include but are not limited to prednisone, prednisolone, methylprednisone, triacmcinolone, betamethasone, dexamethasone, and prodrugs thereof.

In certain embodiments, the second therapeutic agent is a tyrosine kinase inhibitor. Examples of tyrosine kinase inhibitors considered for use in combination therapies of the invention include but are not limited to inhibitors of the following kinases, including, among others: JAK, Syk, JNK/SAPK, MAPK, PI-3K, and/or Ripk2. In certain embodiments, the tyrosine kinase inhibitor is ruxolitinib, tofacitinib, oclactinib, filgotinib, ganotinib, lestaurtinib, momelotinib, pacritinib, upadacitinib, peficitinib, fedratinib, bentamapimod, D-JNKI-1 (XG-102, AM-111), ponatinib, WEHI-345, OD36, GSK583, idelalisib, copanlisib, taselisib, duvelisib, alpelisib, umbralisib, dactolisib, CUDC-907, entospletinib, fostamatinib, or combinations thereof. In certain embodiments, the second therapeutic agent is a Bruton Tyrosine Kinase (BTK) inhibitor. Examples of BTK inhibitors considered for use in combination therapies of the invention include but are not limited to ibrutinib, acalabrutinib, pirtobrutinib, and zanubrutinib.

In certain embodiments, the second therapeutic agent is a receptor kinase inhibitor, including among others, an inhibitor of EGFR or HER2. Examples of receptor kinase inhibitors considered for use in combination therapies of the invention include but are not limited to gefitinib, erlotinib, neratinib, lapatinib, cetuximab, panitumumab, vandetanib, necitumumab, osimertinib, trastuzumab, neratinib, lapatinib, pertuzumab, or combinations thereof.

In certain embodiments, the second therapeutic agent is a modulator of nuclear receptor family of transcription factors, including, among others, an inhibitor of PPAR, RXR, FXR, or LXR. In certain embodiments, the inhibitor is pioglitazone, bexarotene, obeticholic acid, ursodeoxycholic acid, fexaramine, hypocholamide, or combinations thereof.

In certain embodiments, the second therapeutic agent is an HSP90 inhibitor. Examples of HSP90 inhibitors considered for use in combination therapies of the invention include but are not limited to ganetespib, 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010, or combinations thereof.

In certain embodiments, the second therapeutic agent is an adenosine receptor 2A (A₂A) agonist. Examples of adenosine receptor agonists considered for use in combination therapies of the invention include but are not limited to those disclosed in U.S. Pat. No. 9,067,963, which is incorporated herein by reference. In certain embodiments, the adenosine receptor agonist is LNC-3050, LNC-3015, LNC-3047, LNC-3052, or combinations thereof.

In certain embodiments, the second therapeutic agent is selected from disease modifying antirheumatic drugs (DMARDS). Examples of DMARDS considered for use in combination therapies of the invention include but are not limited to tocilizumab, certolizumab, etanercept, adalimumab, anakinra, abatacept, infliximab, rituximab, golimumab, uteskinumab, or combinations thereof.

In certain embodiments, the second therapeutic agent is a phosphodiesterase (PDE) inhibitor. Examples of phosphodiesterase inhibitor considered for use in combination therapies of the invention include but are not limited to apremilast, crisaborole, piclimilast, drotaverine, ibudulast, roflumilast, sildenafil, tadalafil, vardenafil, or combinations thereof.

In certain embodiments, the second therapeutic agent is a neutrophil elastase inhibitor. Examples of neutrophil elastase inhibitors considered for use in combination therapies of the invention include but are not limited to sivelestat.

In certain embodiments, the second therapeutic agent is a modulator of Axl kinase. Examples of modulators of Axl kinase considered for use in combination therapies of the invention include but are not limited to bemcentinib (BGB324 or R428), TP-0903, LY2801653, amuvatinib (MP-470), bosutinib (SKI-606), MGCD 265, ASP2215, cabozantinib (XL184), foretinib (GSK1363089/XL880), and SGI-7079. In certain embodiments, the modulator of Axl kinase is a monoclonal antibody targeting AXL (e.g., YW327.6S2) or an AXL decoy receptor (e.g., GL2I.T), or glesatinib, merestinib, or a dual Flt3-Axl inhibitor such as gilteritinib.

In certain embodiments, the additional therapeutic agent is an anti-cancer agent or chemo-therapeutic agent. Examples of anti-cancer agents considered for use in combination therapies of the invention include but are not limited erlotinib, bortezomib, fulvestrant, sunitib, imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, camptothecin, topotecan, bryostatin, adezelesin, anthracyclin, carzelesin, bizelesin, dolastatin, auristatins, duocarmycin, eleutherobin, taxols such as paclitaxel or docetaxel, cyclophosphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine, and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, edrecolomab, epirubicin, bleomycin, plicamycin, mitomycin, as well as other anticancer antibodies (cetuximab, bevacizumab, ibritumomab, abagovomab, adecatumumab, afutuzumab, alacizumab, alemtuzumab, anatumomab, apolizumab, bavituximab, belimumab, bivatuzumab mertansine, blinatumomab, brentuximab vedotin, cantuzumab mertansine, catumazomab, cetuximab, citatuzumab bogatox, cixutumumab, clivatuzumab tetraxetan, conatumumab, dacetuzumab, daclizumab, detumomab, ecromeximab, edrecolomab, elotuzumab, epratuzumab, ertumaxomab, etaracizumab, farletuzumab, figitumumab, fresolimumab, galiximab, gembatumumab vedotin, gemtuzumab, ibritumomab tiuxetan, inotuzumab ozogamicin, intetumumab, ipilimumab, iratumumab, labetuzumab, lexatumumab, lintuzumab, lucatumumab, lumilisimab, mapatumumab, matuzumab, milatuzumab, mitumomab, nacolomab tafenatox, naptumomab estafenatox, necitumumab, nimotuzumab, ofatumumab, olaratumab, oportuzumab monatox, oregovomab, panitumumab, pemtumomab, pertuzumab, pintumomab, pritumumab, ramucirumab, rilotumumab, robatumumab, rituximab, sibrotuzumab, tacatuzumab tetraxetan, taplitumomab paptox, tenatumomab, ticilimumab, tigatuzumab, tositumomab or ¹³¹I-tositumomab, trastuzumab, tremelimumab, tuocotuzumab celmoleukin, veltuzumab, visilizumab, volocixumab, votumumab, zalutumumab, zanolimumab, IGN-101, MDX-010, ABX-EGR, EMD72000, ior-t1, MDX-220, MRA, H-11 scFv, huJ591, TriGem, TriAb, R3, MT-201, G-250, ACA-125, Onyvax-105, CD:-960, Cea-Vac, BrevaRex AR54, IMC-1C11, GlioMab-H, ING-1, anti-LCG MAbs, MT-103, KSB-303, Therex, KW2871, anti-HMI.24, Anti-PTHrP, 2C₄ antibody, SGN-30, TRAIL-RI MAb, Prostate Cancer antibody, H22xKi-r, ABX-Mai, Imuteran, Monopharm-C), and antibody-drug conjugates comprising any of the above agents (especially auristatins MMAE and MMAF, maytansinoids like DM-1, calicheamycins, or various cytotoxins).

In certain embodiments, the additional therapeutic agent is selected from anastrozole (ARIMIDEX®), bicalutamide (CASODEX®), bleomycin sulfate (BLENOXANE®), busulfan (MYLERAN®), busulfan injection (BUSULFEX®), capecitabine (XELODA®), N4-pentoxycarbonyl-5-deoxy-5-fluorocytidine, carboplatin (PARAPLATIN®), carmustine (BiCNU®), chlorambucil (LEUKERAN®), cisplatin (PLATINOL®), cladribine (LEUSTATIN®), cyclophosphamide (CYTOXAN® or NEOSAR®), cytarabine, cytosine arabinoside (CYTOSAR-U®), cytarabine liposome injection (DEPOCYT®), dacarbazine (DTIC-Dome®), dactinomycin (actinomycin D, COSMEGAN®), daunorubicin hydrochloride (CERUBIDINE®), daunorubicin citrate liposome injection (DAUNOXOME®), dexamethasone, docetaxel (TAXOTERE®), doxorubicin hydrochloride (ADRIAMYCIN®, RUBEX®), etoposide (VEPESID®), fludarabine phosphate (FLUDARA®), 5-fluorouracil (ADRUCIL®, EFUDEX®), flutamide (EULEXIN®), tezacitibine, gemcitabine (difluorodeoxycitidine), hydroxyurea (HYDREA®), idarubicin (IDAMYCIN®), ifosfamide (IFEX®), irinotecan (CAMPTOSAR®), L-asparaginase (ELSPAR®), leucovorin calcium, melphalan (ALKERAN®), 6-mercaptopurine (PURINETHOL®), methotrexate (FOLEX®), mitoxantrone (NOVANTRONE®), gemtuzumab ozogamicin (MYLOTARG™), paclitaxel (TAXOL®), nab-paclitaxel (ABRAXANE®), phoenix (Yttrium90/MX-DTPA), pentostatin, polifeprosan 20 with carmustine implant (GLIADEL®), tamoxifen citrate (NOLVADEX®), teniposide (VUMON®), 6-thioguanine, thiotepa, tirapazamine (TIRAZONE®), topotecan hydrochloride for injection (HYCAMPTIN®), vinblastine (VELBAN®), vincristine (ONCOVIN®), and vinorelbine (NAVELBINE®).

In certain embodiments, the additional therapeutic agent is capable of inhibiting BRAF, MEK, CDK4/6, SHP-2, HDAC, EGFR, MET, mTOR, PI3K or AKT, or a combination thereof. In a particular embodiment, the compounds of the present invention are combined with another therapeutic agent selected from vemurafinib, debrafinib, LGX818, trametinib, MEK162, LEE011, PD-0332991, panobinostat, verinostat, romidepsin, cetuximab, gefitinib, erlotinib, lapatinib, panitumumab, vandetanib, INC280, everolimus, simolimus, BMK120, BYL719 or CLR457, or a combination thereof.

In certain embodiments, the additional therapeutic agent is selected based on the disease or condition that is being treated. For example, in the treatment of melanoma, the additional therapeutic agent is selected from aldesleukin (e.g., PROLEUKIN®), dabrafenib (e.g., TAFINLAR®), dacarbazine, recombinant interferon alfa-2b (e.g., INTRON® A), ipilimumab, trametinib (e.g., MEKINIST®), peginterferon alfa-2b (e.g., PEGINTRON®, SYLATRON™), vemurafenib (e.g., ZELBORAF®)), and ipilimumab (e.g., YERVOY®).

For the treatment of ovarian cancer, the additional therapeutic agent is selected from doxorubicin hydrochloride (Adriamycin®), carboplatin (PARAPLATIN®), cyclophosphamide (CYTOXAN®, NEOSAR®), cisplatin (PLATINOL®, PLATINOL-AQ®), doxorubicin hydrochloride liposome (DOXIL®, DOX-SL®, EVACET®, LIPODOX®), gemcitabine hydrochloride (GEMZAR®), topotecan hydrochloride (HYCAMTIN®), and paclitaxel (TAXOL®).

For the treatment of thyroid cancer, the additional therapeutic agent is selected from doxorubicin hydrochloride (Adriamycin®), cabozantinib-S-malate (COMETRIQ®), and vandetanib (CAPRELSA®).

For the treatment of colon cancer, the additional therapeutic agent is selected from fluorouracil (e.g., ADRUCIL®, EFUDEX®, FLUOROPLEX®), bevacizumab (AVASTIN®), irinotecan hydrochloride (CAMPTOSTAR®), capecitabine (XELODA®), cetuximab (ERBITUX®), oxaliplatin (ELOXATIN®), leucovorin calcium (WELLCOVORIN®), regorafenib (STIVARGA®), panitumumab (VECTIBIX®), and ziv-aflibercept (ZALTRAP®).

For the treatment of lung cancer, the additional therapeutic agent is selected from methotrexate, methotrexate LPF (e.g., FOLEX®, FOLEX PFS®, Abitrexate®, MEXATE®, MEXATE-AQ®), paclitaxel (TAXOL®), paclitaxel albumin-stabilized nanoparticle formulation (ABRAXANE®), afatinib dimaleate (GILOTRIF®), pemetrexed disodium (ALIMTA®), bevacizumab (AVASTIN®), carboplatin (PARAPLATIN®), cisplatin (PLATINOL®, PLATINOL-AQ®), crizotinib (XALKORI®), erlotinib hydrochloride (TARCEVA®), gefitinib (TRESSA®), and gemcitabine hydrochloride (GEMZAR®).

For the treatment of pancreatic cancer, the other therapeutic agent may be selected from fluorouracil (ADRUCIL®), EFUDEX®, FLUOROPLEX®), erlotinib hydrochloride (TARCEVA®), gemcitabine hydrochloride (GEMZAR®), and mitomycin or mitomycin C (MITOZYTREX™, MUTAMYCIN®).

For the treatment of cervical cancer, the additional therapeutic agent is selected from bleomycin (BLENOXANE®), cisplatin (PLATINOL®, PLATINOL-AQ®) and topotecan hydrochloride (HYCAMTIN®).

For the treatment of head and neck cancer, the additional therapeutic agent is selected from methotrexate, methotrexate LPF (e.g., FOLEX®, FOLEX PFS®, Abitrexate®, MEXATE®, MEXATE-AQ®), fluorouracil (ADRUCIL®, EFUDEX®, FLUOROPLEX®), bleomycin (BLENOXANE®), cetuximab (ERBITUX®), cisplatin (PLATINOL®, PLATINOL-AQ®) and docetaxel (TAXOTERE®).

For the treatment of leukemia, including chronic myelomonocytic leukemia (CMML), the additional therapeutic agent is selected from bosutinib (BOSULIF®), cyclophosphamide (CYTOXAN®, NEOSAR®), cytarabine (CYTOSAR-U®, TARABINE PFS®), dasatinib (SPRYCEL®), imatinib mesylate (GLEEVEC®), ponatinib (ICLUSIG®), nilotinib (TASIGNA®) and omacetaxine mepesuccinate (SYNRIBO®).

In some instances, patients may experience allergic reactions to the compounds of the present invention and/or other anti-cancer agent(s) during or after administration. Therefore, anti-allergic agents may be administered to minimize the risk of an allergic reaction. Suitable anti-allergic agents include corticosteroids, such as dexamethasone (e.g., DECADRON®), beclomethasone (e.g., BECLOVENT®), hydrocortisone (also known as cortisone, hydrocortisone sodium succinate, hydrocortisone sodium phosphate; e.g., ALA-CORT®, hydrocortisone phosphate, Solu-CORTEF®, HYDROCORT Acetate® and LANACORT®), prednisolone (e.g., DELTA-Cortel®, ORAPRED®, PEDIAPRED® and PRELONE®), prednisone (e.g., DELTASONE®, LIQUID RED®, METICORTEN® and ORASONE®), methylprednisolone (also known as 6-methylprednisolone, methylprednisolone acetate, methylprednisolone sodium succinate; e.g., DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

In other instances, patients may experience nausea during and after administration of the compound of the present invention and/or other anti-cancer agent(s). Therefore, anti-emetics may be administered in preventing nausea (upper stomach) and vomiting. Suitable anti-emetics include aprepitant (EMEND®), ondansetron (ZOFRAN®), granisetron HCl (KYTRIL®), lorazepam (ATIVAN®. dexamethasone (DECADRON®), prochlorperazine (COMPAZINE®), casopitant (REZONIC® and Zunrisa®), and combinations thereof.

In yet other instances, medication to alleviate the pain experienced during the treatment period is prescribed to make the patient more comfortable. Common over-the-counter analgesics, such TYLENOL®, are often used. Opioid analgesic drugs such as hydrocodone/paracetamol or hydrocodone/acetaminophen (e.g., VICODIN®), morphine (e.g., ASTRAMORPH® or AVINZA®), oxycodone (e.g., OXYCONTIN® or PERCOCET®), oxymorphone hydrochloride (OPANA®), and fentanyl (e.g., DURAGESIC®) are also useful for moderate or severe pain.

Furthermore, cytoprotective agents (such as neuroprotectants, free-radical scavengers, cardioprotectors, anthracycline extravasation neutralizers, nutrients and the like) may be used as an adjunct therapy to protect normal cells from treatment toxicity and to limit organ toxicities. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

In yet another aspect, a compound of the present invention may be used in combination with known therapeutic processes, for example, with the administration of hormones or in radiation therapy. In certain instances, a compound of the present invention may be used as a radiosensitizer, especially for the treatment of tumors which exhibit poor sensitivity to radiotherapy.

The doses and dosage regimen of the active ingredients used in the combination therapy may be determined by an attending clinician. In certain embodiments, the compound described herein (e.g., a compound of Formula I, I-A, I-B, I-C, II, or other compounds in Section I) and the additional therapeutic agent(s) are administered in doses commonly employed when such agents are used as monotherapy for treating the disease or condition. In other embodiments, the compound described herein (e.g., a compound of Formula I, I-A, I-B, I-C, II, or other compounds in Section I) and the additional therapeutic agent(s) are administered in doses lower than the doses commonly employed when such agents are used as monotherapy for treating the disease or condition. In certain embodiments, the compound described herein (e.g., a compound of Formula I, I-A, I-B, I-C, II, or other compounds in Section I) and the additional therapeutic agent(s) are present in the same composition, which is suitable for oral administration.

In certain embodiments, the compound described herein (e.g., a compound of Formula I, I-A, I-B, I-C, II, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

### IV. PHARMACEUTICAL COMPOSITIONS AND DOSING CONSIDERATIONS

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (e.g., a compound of Formula I, I-A, I-B, I-C, II, or other compounds in section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (e.g., capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a imidazopyrimidine compound or related compound described herein in a therapeutically effective amount for the treatment of a disease or condition described herein.

### IV. MEDICAL KITS

Another aspect of the invention provides a medical kit comprising, for example, (i) a compound described herein, and (ii) instructions for use according to a method described herein.

### V. ENUMERATED EMBODIMENTS

The following exemplary embodiments are provided:

Embodiment 1 provides a compound of formula I-1:

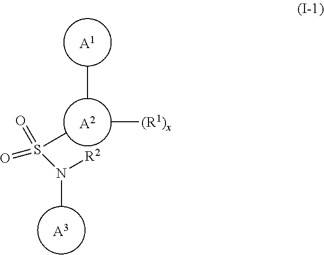

or a pharmaceutically acceptable salt thereof, wherein:
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, C₃₋₇ halocycloalkyl, C₃₋₇ hydroxycycloalkyl, —O—C₃₋₇ cycloalkyl, nitro, —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶;
A² is a pyridinylene, pyridazinylene, pyrimidinylene, or phenylene;
A³ is

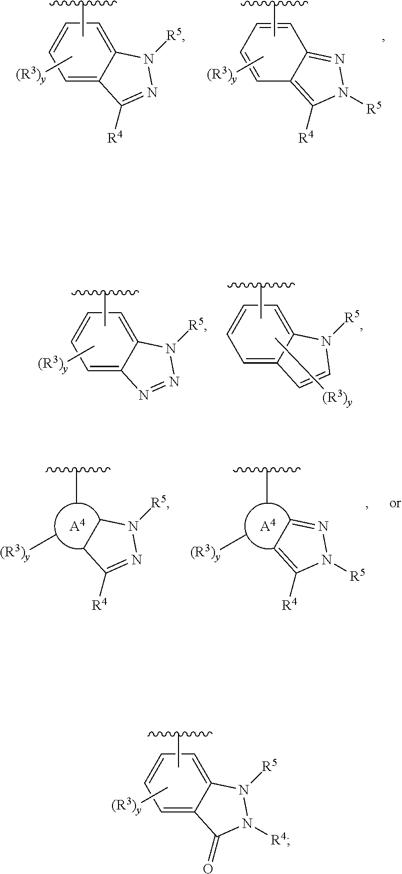

A⁴ is a 6-membered aromatic ring containing 1 nitrogen atom;
y is 0, 1, or 2; and
m, n, and x are independently 0, 1, or 2;
provided that if A² is phenylene, then y is 1 or 2 and at least one occurrence of R³ is C₁₋₆ alkoxyl, C₁₋₆ deuteroalkoxyl, or C₃₋₇ cycloalkyl.

Embodiment 2 provides the compound of embodiment 1, wherein the compound is a compound of Formula I.

Embodiment 3 provides the compound of embodiment 1 or 2, wherein R² is hydrogen.

Embodiment 4 provides the compound of embodiment 1 or 2, wherein R² is C₁₋₄ alkyl.

Embodiment 5 provides the compound of any one of embodiments 1-3, wherein A² is a pyridinylene.

Embodiment 6 provides the compound of any one of embodiments 1-3, wherein A² is pyridazinylene or pyrimidinylene.

Embodiment 7 provides the compound of embodiment 1, wherein the compound is a compound of Formula Ia-1 or a pharmaceutically acceptable salt thereof:

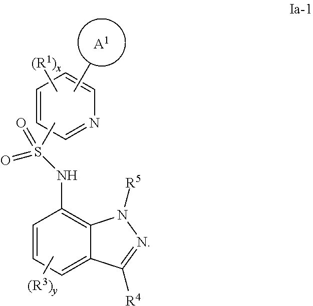

Embodiment 8 provides the compound of embodiment 1, wherein the compound is a compound of Formula Ib-1 or a pharmaceutically acceptable salt thereof:

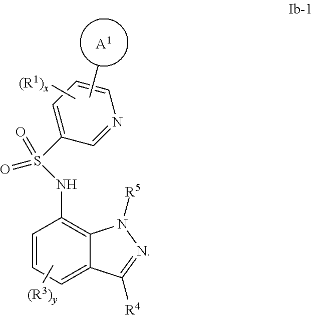

Embodiment 9 provides the compound of embodiment 1, wherein the compound is a compound of Formula Ic-1 or a pharmaceutically acceptable salt thereof:

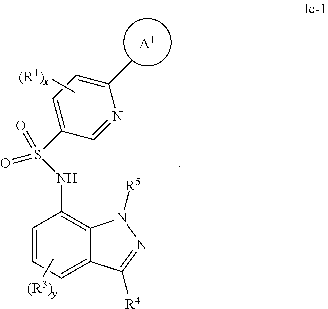

Embodiment 10 provides the compound of embodiment 1, wherein the compound is a compound of Formula Id-1 or a pharmaceutically acceptable salt thereof:

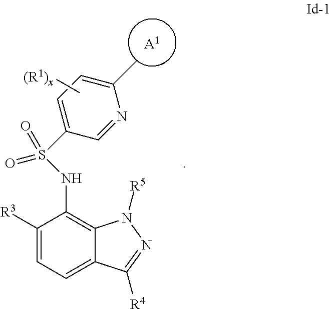

Embodiment 11 provides the compound of any one of embodiments 1-8, wherein y is 1.

Embodiment 12 provides the compound of any one of embodiments 1-10, wherein x is 0.

Embodiment 13 provides the compound of any one of embodiments 1-11, wherein A¹ is a 5-membered heteroaryl containing 1, 2, or 3 heteroatoms independently selected from oxygen, nitrogen, and sulfur, wherein the heteroaryl is substituted with n occurrences of R⁶.

Embodiment 14 provides the compound of any one of embodiments 1-11, wherein A¹ is pyrazolyl, imidazolyl, oxazolyl, isoxazolyl, isothiazolyl, pyrrolyl, or furanyl, each of which is substituted with n occurrences of R⁶.

Embodiment 15 provides the compound of any one of embodiments 1-11, wherein A¹ is pyrazolyl substituted with n occurrences of R⁶.

Embodiment 16 provides the compound of any one of embodiments 1-11, wherein A¹ is

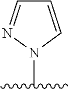 [SMILES: *n1cccn1]

substituted with n occurrences of R⁶.

Embodiment 17 provides the compound of any one of embodiments 1-15, wherein n is 1.

Embodiment 18 provides the compound of any one of embodiments 1-15, wherein n is 2.

Embodiment 19 provides the compound of any one of embodiments 1-11, wherein A¹ is

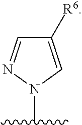

Embodiment 20 provides the compound of any one of embodiments 1-18, wherein R⁶ is C₁₋₆ haloalkyl.

Embodiment 21 provides the compound of any one of embodiments 1-18, wherein R⁶ is —CF₃.

Embodiment 22 provides the compound of any one of embodiments 1-18, wherein R⁶ is C₃₋₇ cycloalkyl.

Embodiment 23 provides the compound of any one of embodiments 1-18, wherein R⁶ is cyclopropyl.

Embodiment 24 provides the compound of any one of embodiments 1-18, wherein R⁶ is halo.

Embodiment 25 provides the compound of any one of embodiments 1-18, wherein R⁶ represents independently for each occurrence C₁₋₆ haloalkyl, —C(O)R⁷, or —C(O)N(R⁸)(R⁹).

Embodiment 26 provides the compound of any one of embodiments 1-24, wherein R⁵ is C₁₋₄ alkyl.

Embodiment 27 provides the compound of any one of embodiments 1-24, wherein R⁵ is methyl.

Embodiment 28 provides the compound of any one of embodiments 1-26, wherein R⁴ is hydrogen.

Embodiment 29 provides the compound of any one of embodiments 1-27, wherein R³ is C₁₋₆ alkyl.

Embodiment 30 provides the compound of any one of embodiments 1-27, wherein R³ is ethyl.

Embodiment 31 provides the compound of any one of embodiments 1-27, wherein R³ is C₃₋₇ cycloalkyl.

Embodiment 32 provides the compound of any one of embodiments 1-27, wherein R³ is cyclopropyl.

Embodiment 33 provides the compound of any one of embodiments 1-27, wherein R³ is C₁₋₆ alkoxyl.

Embodiment 34 provides the compound of any one of embodiments 1-27, wherein R³ is methoxy.

Embodiment 35 provides a compound represented by Formula II-1:

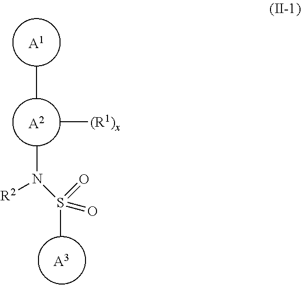

or a pharmaceutically acceptable salt thereof, wherein:
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, C₃₋₇ halocycloalkyl, C₃₋₇ hydroxycycloalkyl, nitro, —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶;
A² is a pyridinylene, pyridazinylene, or pyrimidinylene;
A³ is

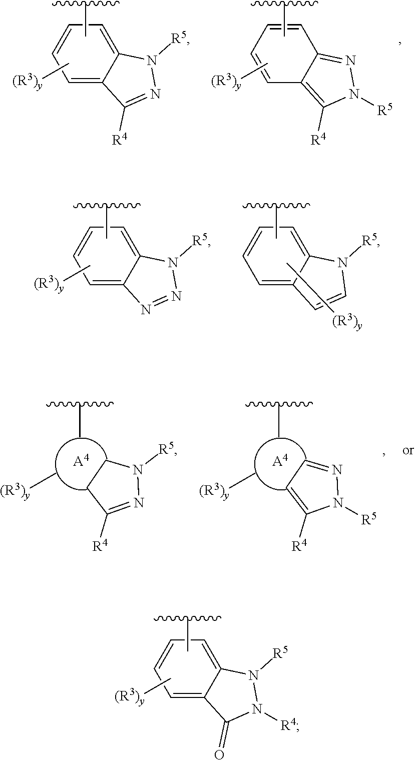

A⁴ is a 6-membered aromatic ring containing 1 nitrogen atom;
y is 1 or 2; and
m, n, and x are independently 0, 1, or 2;
provided at least one occurrence of R³ is C₁₋₆ alkoxyl, C₁₋₆ deuteroalkoxyl, or C₃₋₇ cycloalkyl.

Embodiment 36 provides the compound of embodiment 35, wherein the compound is a compound of Formula II-1.

Embodiment 37 provides the compound of embodiment 35 or 36, wherein R² is hydrogen.

Embodiment 38 provides the compound of embodiment 35 or 36, wherein R² is C₁₋₄ alkyl.

Embodiment 39 provides the compound of any one of embodiments 35-38, wherein A² is a pyridinylene.

Embodiment 40 provides the compound of any one of embodiments 35-39, wherein A¹ is a 5-membered heteroaryl containing 1, 2, or 3 heteroatoms independently selected from oxygen, nitrogen, and sulfur, wherein the heteroaryl is substituted with n occurrences of R⁶.

Embodiment 41 provides the compound of any one of embodiments 35-39, wherein A¹ is pyrazolyl, imidazolyl, oxazolyl, isoxazolyl, isothiazolyl, pyrrolyl, or furanyl, each of which is substituted with n occurrences of R⁶.

Embodiment 42 provides the compound of any one of embodiments 35-39, wherein A¹ is pyrazolyl substituted with n occurrences of R⁶.

Embodiment 43 provides the compound of any one of embodiments 35-39, wherein A¹ is

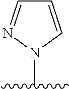 [SMILES: *n1cccn1]

substituted with n occurrences of R⁶.

Embodiment 44 provides the compound of any one of embodiments 35-43, wherein A³ is

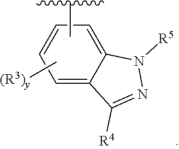

Embodiment 45 provides a compound in Table 1 or 2, or a pharmaceutically acceptable salt thereof.

Embodiment 46 provides a pharmaceutical composition comprising a compound of any one of embodiments 1-45 and a pharmaceutically acceptable carrier.

Embodiment 47 provides a method for treating a disease or condition mediated by MALT1, comprising administering to a subject in need thereof a therapeutically effective amount of a compound of any one of embodiments 1-45 to treat the disease or condition.

Embodiment 48 provides the method of embodiment 47, wherein said disease or condition mediated by MALT1 is a proliferative disorder.

Embodiment 49 provides the method of embodiment 47, wherein said disease or condition mediated by MALT1 is an inflammatory disorder.

Embodiment 50 provides the method of embodiment 47, wherein said disease or condition mediated by MALT1 is an autoimmune disorder.

Embodiment 51 provides the method of embodiment 47, wherein said disease or condition mediated by MALT1 is selected from cancer, neoplasia, chronic inflammatory disorder, acute inflammatory disorder, auto-inflammatory disorder, autoimmune disorder, fibrotic disorder, metabolic disorder, cardiovascular disorder, cerebrovascular disorder, myeloid cell-driven hyper-inflammatory response in COVID-19 infection, and a combination thereof.

Embodiment 52 provides the method of embodiment 47, wherein said disease or condition mediated by MALT1 is cancer.

Embodiment 53 provides the method of embodiment 52, wherein the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia.

Embodiment 54 provides the method of embodiment 47, wherein said disease or condition mediated by MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

Embodiment 55 provides the method of embodiment 47, wherein said disease or condition mediated by MALT1 is multiple sclerosis, ankylosing spondylitis, arthritis, osteoarthritis, juvenile arthritis, reactive arthritis, rheumatoid arthritis, psoriatic arthritis, acquired immunodeficiency syndrome (AIDS), Coeliac disease, psoriasis, chronic graft-versus-host disease, acute graft-versus-host disease, Crohn's disease, inflammatory bowel disease, multiple sclerosis, systemic lupus erythematosus, Celiac Sprue, idiopathic thrombocytopenic thrombotic purpura, myasthenia gravis, Sjogren's syndrome, scleroderma, ulcerative colitis, asthma, uveitis, rosacea, dermatitis, alopecia areata, vitiligo, arthritis, Type 1 diabetes, lupus erythematosus, systemic lupus erythematosus, lashimoto's thyroiditis, myasthenia gravis, nephrotic syndrome, eosinophilia fasciitis, hyper IgE syndrome, lepromatous leprosy, sezary syndrome, idiopathic thrombocytopenia purpura, restenosis following angioplasty, a tumor, or artherosclerosis.

Embodiment 56 provides the method of embodiment 47, wherein said disease or condition mediated by MALT1 is allergic rhinitis, nasal inflammation, asthma, chronic obstructive pulmonary disease (COPD), bronchitis, emphysema, chronic eosinophilic pneumonia, adult respiratory distress syndrome, sinusitis, allergic conjunctivitis, idiopathic pulmonary fibrosis, atopic dermatitis, asthma, allergic rhinitis, arthritis, inflammatory bowel disease, Crohn's disease, ulcerative colitis, multiple sclerosis, endometriosis, eczema, psoriasis, rosacea, or lupus erythematosus.

Embodiment 57 provides the method of any one of embodiments 47-56, wherein the subject is a human.

Embodiment 58 provides a method of inhibiting the activity of MALT1, comprising contacting a MALT1 with an effective amount of a compound of any one of embodiments 1-45 to Inhibit the activity of said MALT1.

### EXAMPLES

The invention now being generally described, will be more readily understood by reference to the following examples, which are included merely for purposes of illustrating certain aspects and embodiments of the present invention, and are not intended to limit the invention.

The following general procedures were used in certain instances. Examples below may refer to one of the following general procedures. NMR chemical shift data are presented in ppm values.

General Procedure A: For Coupling a Sulfonyl Chloride and an Amine

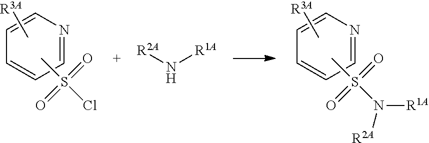

To a solution of amine compound (1.0 eq.) in pyridine is added the sulfonyl chloride compound (1.2-2.0 eq.) and 4-(dimethylamino)pyridine (0.1 eq.) at rt. The resulting reaction mixture is heated at 50° C. for 20 h. Then, the reaction mixture solvent is evaporated under reduced pressure and the resulting residue is partitioned between ethyl acetate (EtOAc) and water. The organic layer of the mixture is isolated and dried over magnesium sulfate, filtered and concentrated in vacuum. The resulting residue is purified by preparative HPLC.

General Procedure B: For Coupling a 2-Chloroheteroaryl Sulfonamide and A Pyrazole Compound

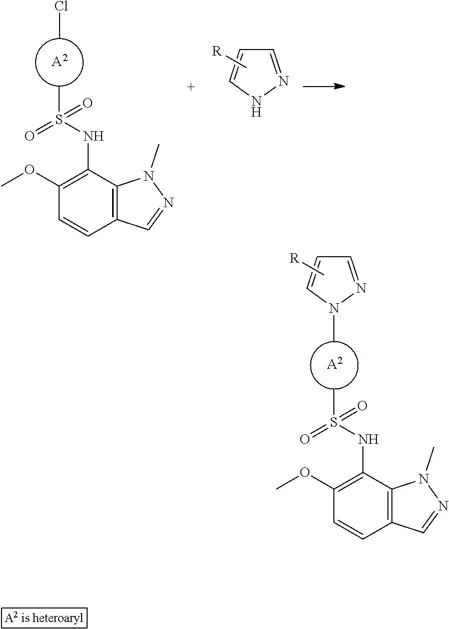

A mixture of chloro-heteroaryl sulfonamide (1 eq.), pyrazole compound (2 eq.), N1, N2-dimethylcyclohexane-1,2-diamine (2 eq.), CuI (1 eq.) and Cs₂CO₃ (3 eq.) in DMSO is stirred for 16 h at 150° C. Then, the reaction solvent is partitioned between EtOAc and water. The organic layer of the mixture is isolated and dried over magnesium sulfate, filtered, and concentrated in vacuum. The resulting residue is purified by preparative HPLC.

General Procedure c: For Coupling of a Pyrazol Carboxylic Acids and an Amine

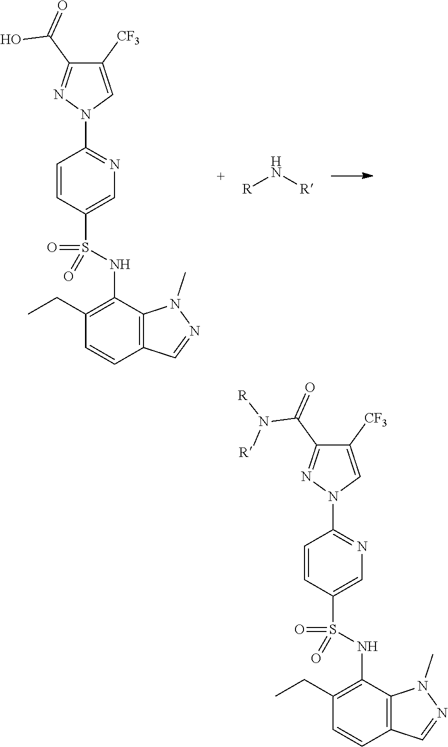

DIPEA (5.0 eq.) was added to a stirred mixture of carboxylic acid (1.0 eq.), amine (2.0 eq.) and HATU (1.5 eq.) in DMF and stirred at ambient temperature for 16 h. The mixture was diluted with EtOAc and washed with aqueous NaHCO₃ and brine, dried over magnesium sulfate and concentrated in vacuo. The residue was submitted for purification by preparative HPLC.

General Procedure D: For Coupling of a 2-Chloropyridine Sulfonamide and A Boric Acid Ester

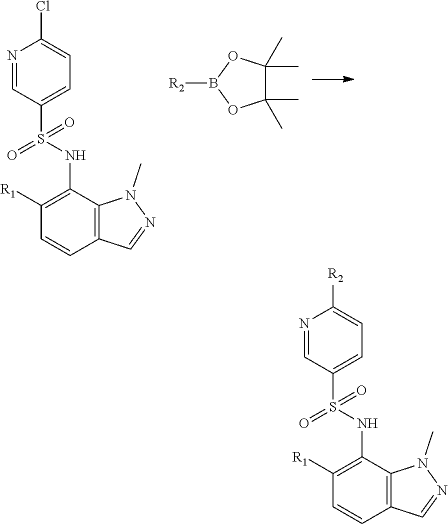

A mixture of 2-chloropyridine sulfonamide (1 eq.), boric acid ester (1.5 eq.), Na₂CO₃ (2 eq.) and Pd(PPh₃)₂Cl₂ (0.1 eq.) in H₂O and dioxane was stirred for 16 h at 110° C. under a nitrogen atmosphere. The mixture was partitioned between ethyl acetate and water. The organic phase was dried over magnesium sulfate, filtered and concentrated in vacuo. The residue was submitted for purification by preparative HPLC.

General Procedure E: For Coupling of 2-Chloropyridines and Boronic Esters or Acids

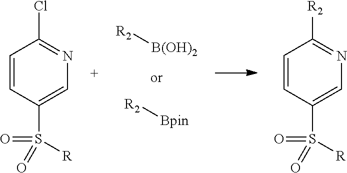

Palladium(II) acetate (0.06 eq.) was added to a mixture of 2-chloropyridine derivative, triphenylphosphine (0.24 eq.), aqueous potassium carbonate (2 M, 2.80 eq.) and boronic ester or acid (1.1 eq.) in ethylene glycol dimethyl ether and heated to 150° C. for 15 min under microwave irridiation. The mixture was cooled to room temperature, diluted with EtOAc, washed with aqueous NaHCO₃ solution and water. The organic phase was dried over magnesium sulfate and concentrated under reduced pressure. The residue was submitted to preparative HPLC for purification.

### Example 1—Synthesis of 6-Cyclopropyl-1-Methyl-7-Nitroindazole

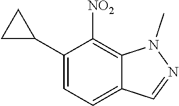 [SMILES: Cn1ncc2ccc(C3CC3)c([N+](=O)[O-])c21]

To a stirred mixture of 6-chloro-1-methyl-7-nitroindazole (5.0 g, 24 mmol, 1.0 eq.) and cyclopropylboronic acid (6.1 g, 71 mmol, 3.0 eq.) in 1,4-dioxane (30 mL) and H₂O (4 mL) was added K₂CO₃ (6.5 g, 47 mmol, 2.0 eq.) and Pd(dppf)Cl₂ (0.86 g, 1.2 mmol, 0.05 eq.) in portions at 100° C. under nitrogen. The reaction mixture was allowed to cool down to rt, and then extracted with EtOAc (4×50 mL). The combined organic layers were dried over anhydrous Na₂SO₄, and evaporated under reduced pressure. The residue was purified by silica gel column chromatography, eluted with PE/EA (1:1) to afford the title compound (3.5 g, 41%) as a brown solid. (ES, m/z): [M+H]⁺ 218; ¹H NMR (400 MHz, CDCl₃) δ 0.70-0.80 (m, 2H), 1.00-1.2 (m, 2H), 2.10-2.25 (m, 1H), 4.00 (s, 3H), 6.82 (d, J=8.4 Hz, 1H), 7.74 (d, J=8.4 Hz, 1H), 8.01 (s, 1H).

### Example 2—Synthesis of 6-Cyclopropyl-1-Methyl-Indazol-7-Amine

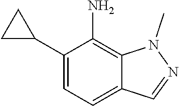

A mixture of 6-cyclopropyl-1-methyl-7-nitroindazole (3.5 g, 9 mmol, 1.0 eq.) and Pd/C (2 g, 19 mmol, 2.0 eq.) in EtOAc was stirred for 3 h at room temperature under hydrogen. The resulting reaction mixture was filtered, and the solid was washed with EtOAc (2×200 mL). The filtrate was concentrated under reduced pressure, and the resulting residue was purified by reversed-phase flash chromatography with the following conditions: column: C18 silica gel; mobile phase: MeCN in water (0.1% TFA), 20% to 50% gradient in 20 min; detector: UV 254 nm. The resulting mixture was concentrated under reduced pressure to afford the title compound (2.7 g, 45%) as a brown solid. (ES, m/z): [M+H]⁺188; ¹H NMR (400 MHz, CDCl₃) δ 0.40-0.60 (m, 2H), 0.85-1.00 (m, 2H), 1.75-1.90 (m, 1H), 4.29 (s, 3H), 5.00 (s, 2H), 6.71 (d, J=8.4 Hz, 1H), 6.91 (d, J=8.4 Hz, 1H), 7.76 (s, 1H).

### Example 3—Synthesis of 7-Nitro-1H-Indazol-6-yl Trifluoromethanesulfonate

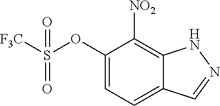 [SMILES: O=[N+]([O-])c1c(OS(=O)(=O)C(F)(F)F)ccc2cn[nH]c12]

To a stirred solution of 7-nitro-1H-indazol-6-ol (5 g, 28 mmol, 1.0 eq.) and TEA (7.8 mL, 56 mmol, 2.0 eq.) in CH₂Cl₂ (DCM) (30 mL) was added trifluoromethanesulfonyl chloride (4.5 mL, 42 mmol, 1.5 eq.) dropwise at 0° C. under nitrogen. The resulting reaction mixture was stirred for 2 h at rt, and then diluted with water (30 mL). The resulting mixture was extracted with DCM (3×30 mL). The combined organic layers were washed with brine (2×20 mL), dried over anhydrous Na₂SO₄, and concentrated under reduced pressure. The residue was purified by reverse flash chromatography with the following conditions: column: C18 silica gel; mobile phase: MeCN in water (0.1% FA), 0% to 100% gradient in 30 min to afford the title compound (1.7 g, 19%) as a yellow solid. (ES, m/z): [M+H]⁺312.

### Example 4—Synthesis of 6-[2-(Tert-Butyldimethylsilyl)Ethynyl]-7-Nitro-1H-Indazole

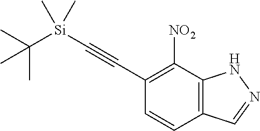 [SMILES: CC(C)(C)[Si](C)(C)C#Cc1ccc2cn[nH]c2c1[N+](=O)[O-]]

To a stirred solution of 7-nitro-1H-indazol-6-yl trifluoromethanesulfonate (1.5 g, 4.8 mmol, 1.0 eq.), Et₃N (3.4 mL, 24 mmol, 5.0 eq.) and CuI (184 mg, 1.0 mmol, 0.2 eq.) in MeCN (10 mL) were added Pd(PPh₃)₂Cl₂ (338 mg, 0.5 mmol, 0.1 eq.) and tert-butyl(ethynyl)dimethylsilane (4.5 mL, 24 mmol, 5.0 eq.). The resulting reaction mixture was stirred 16 h at room temperature under nitrogen, and then diluted with water (20 mL) and extracted with EtOAc (3×10 mL). The combined organic layers were washed with brine (2×30 mL), dried over anhydrous Na₂SO₄, and concentrated under reduced pressure. The residue was purified by reverse flash chromatography with the following conditions: column: C18 silica gel; mobile phase: MeCN in water (0.1% FA), 0% to 100% gradient in 30 min; detector: UV 254 nm to afford the title compound (700 mg, 43%) as a yellow solid. (ES, m/z): [M+H]⁺302.

### Example 5—Synthesis of 6-[2-(Tert-Butyldimethylsilyl)Ethynyl]-1-Methyl-7-Nitroindazole

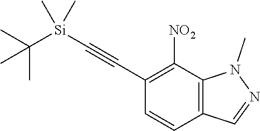 [SMILES: Cn1ncc2ccc(C#C[Si](C)(C)C(C)(C)C)c([N+](=O)[O-])c21]

To a stirred solution of 6-[2-(tert-butyldimethylsilyl)ethynyl]-7-nitro-1H-indazole (700 mg, 2.3 mmol, 1.0 eq.) and Cs₂CO₃ (1.5 g, 4.6 mmol, 2.0 eq.) in DMF (10 mL) was added CH₃I (0.29 mL, 4.6 mmol, 2.0 eq.). The resulting reaction mixture was stirred for 2 h at room temperature under nitrogen. The resulting mixture was diluted with water (50 mL). The resulting mixture was extracted with EtOAc (3×20 mL). The combined organic layers were washed with brine (2×30 mL), dried over anhydrous Na₂SO₄, and concentrated under reduced pressure. The residue was purified by reverse flash chromatography with the following conditions: column: C18 silica gel; mobile phase: MeCN in water (0.1% FA), 0% to 100% gradient in 30 min; detector: UV 254 nm to afford the title compound (280 mg, 34%) as a yellow solid. (ES, m/z): [M+H]⁺ 316; ¹H NMR (400 MHz, DMSO-d₆) δ 0.20 (s, 6H), 0.98 (s, 9H), 3.93 (s, 3H), 7.38 (d, J=8.4 Hz, 1H), 8.09 (d, J=8.4 Hz, 1H), 8.37 (s, 1H).

### Example 6—Synthesis of 6-[2-(Tert-Butyldimethylsilyl)Ethynyl]-1-Methylindazol-7-Amine

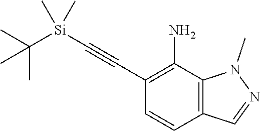 [SMILES: Cn1ncc2ccc(C#C[Si](C)(C)C(C)(C)C)c(N)c21]

To a stirred solution of 6-[2-(tert-butyldimethylsilyl)ethynyl]-1-methyl-7-nitroindazole (330 mg, 1.0 mmol, 1.0 eq.) and NH₄Cl (560 mg, 10.5 mmol, 10 eq.) in H₂O (5 mL) and EtOH (5 mL) was added Fe (584 mg, 10.5 mmol, 10 eq.). The resulting reaction mixture was stirred for 6 h at room temperature under nitrogen, and then was filtered. The solid was washed with EtOAc (3×10 mL), and the filtrate was partitioned and extracted with EtOAc (10 mL). The combined organic layers were dried over anhydrous Na₂SO₄, and concentrated under vacuum to afford the title intermediate (260 mg, 69.65%) as a light yellow oil. (ES, m/z): [M+H]⁺ 286.

### Example 7—Synthesis of 6-Methoxy-1-Methyl-7-Nitroindazole

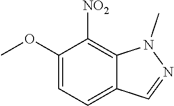

A solution of 6-chloro-1-methyl-7-nitroindazole (300 mg, 1.4 mmol, 1.0 eq.) and MeONa (766 mg, 14 mmol, 10 eq.) in MeOH (10 mL) was stirred for 3 h at 60° C. The resulting reaction mixture was allowed to cool to rt, and diluted with water (50 mL). The resulting mixture was extracted with EtOAc (50 mL). The organic layer was washed with brine, dried over anhydrous Na₂SO₄, and concentrated under reduced pressure to afford the title compound (250 mg, 77%) as a yellow solid. (ES, m/z): [M+H]⁺ 208.

### Example 8—Synthesis of 6-Methoxy-1-Methylindazol-7-Amine

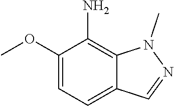

A solution of 6-methoxy-1-methyl-7-nitroindazole (230 mg, 1.1 mmol, 1.0 eq.) and Pd/C (130 mg, 1.2 mmol, 1.1 eq.) in MeOH (5 mL) was stirred for 2 h at room temperature under hydrogen. The resulting reaction mixture was then filtered and the solid material was washed with MeOH (3×10 mL). The filtrate was concentrated under reduced pressure to afford the title compound (195 mg, 89%) as an off-white oil. (ES, m/z): [M+H]⁺ 178.

### Example 9—Synthesis of 6-Ethoxy-1-Methyl-7-Nitroindazole

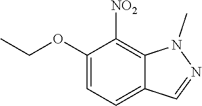 [SMILES: CCOc1ccc2cnn(C)c2c1[N+](=O)[O-]]

6-chloro-1-methyl-7-nitroindazole (400 mg, 1.9 mmol, 1.0 eq.) was treated with sodium ethanolate (1.29 g, 19 mmol, 10 eq.) in EtOH (5 mL) in a similar procedure as that reported for 6-methoxy-1-methyl-7-nitroindazole to afford the title compound (465 mg, 63%) as a white solid. (ES, m/z): [M+H]⁺ 222.

### Example 10—Synthesis of 6-Ethoxy-1-Methylindazol-7-Amine

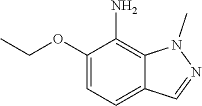

The title compound was obtained through a similar Fe/NH₄Cl reduction as reported for 6-methoxy-1-methylindazol-7-amine using 6-ethoxy-1-methyl-7-nitroindazole instead. A white solid was obtained (165 mg, 41%). (ES, m/z): [M+H]⁺ 192.

### Example 11—Synthesis of 6-Isopropoxy-1-Methyl-7-Nitro-1H-Indazole

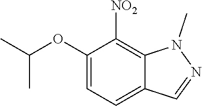 [SMILES: CC(C)Oc1ccc2cnn(C)c2c1[N+](=O)[O-]]

6-chloro-1-methyl-7-nitroindazole (400 mg, 1.9 mmol, 1.0 eq.) was treated with sodium propan-2-olate (1.6 g, 19 mmol, 10 eq.) in isopropyl alcohol (20 mL) in a similar procedure as that reported for 6-methoxy-1-methyl-7-nitroindazole to afford the title compound (410 mg, 92%) as a white solid. (ES, m/z): [M+H]⁺ 236.

### Example 12—Synthesis of 6-Isopropoxy-1-Methyl-1H-Indazol-7-Amine

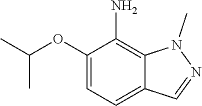

A mixture of 6-isopropoxy-1-methyl-7-nitroindazole (200 mg, 0.85 mmol, 1.0 eq.) and Pd/C (181 mg, 1.7 mmol, 2.0 eq.) in MeOH (10 mL) was stirred for 2 h at room temperature under hydrogen. The reaction was quenched by the addition of MeOH (10 mL) at rt. The resulting mixture was filtered and the solid was washed with MeOH (3×10 mL). The filtrate was concentrated under reduced pressure to afford the title compound (142 mg, 81%) as a brown solid. (ES, m/z): [M+H]⁺ 206.

### Example 13—Synthesis of 2-(7-Amino-1-Methyl-1H-Indazol-6-yl)Acetonitrile

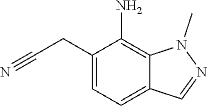

The title compound was obtained through a similar Fe/NH₄Cl reduction as reported for 6-methoxy-1-methylindazol-7-amine using 2-(1-methyl-7-nitroindazol-6-yl)acetonitrile instead. A red-brown solid was obtained (120 mg, 59%). (ES, m/z): [M+H]⁺ 187.

### Example 14—Synthesis of N,N,1-Trimethyl-7-Nitroindazol-6-Amine

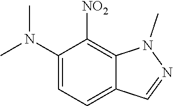 [SMILES: CN(C)c1ccc2cnn(C)c2c1[N+](=O)[O-]]

To a stirred solution of 6-chloro-1-methyl-7-nitroindazole (1 g, 4.7 mmol, 1.0 eq.) in dimethylamine (2 M in DMF, 10 mL) was added K₂CO₃ (1.3 g, 9.5 mmol, 2.0 eq.). The resulting reaction mixture was stirred for 2 h at 150° C. under nitrogen, cooled to rt, and then diluted with water (50 mL). The resulting mixture was extracted with EtOAc (3×20 mL), the combined organic layers were washed with brine (2×30 mL), dried over anhydrous Na₂SO₄, and concentrated under reduced pressure. The residue was purified by reversed phase flash chromatography with the following conditions: column: C18 silica gel; mobile phase: MeCN in Water (0.1% TFA), 20% to 40% gradient in 10 min; detector: UV 254 nm to afford the title compound (500 mg, 48%) as a yellow oil. (ES, m/z): [M+H]⁺ 221; ¹H NMR (400 MHz, DMSO-d₆) δ 2.87 (s, 6H), 3.85 (s, 3H). 7.13 (d, J=8.8 Hz, 1H), 7.86 (d, J=8.8 Hz, 1H), 8.10 (s, 1H).

### Example 15—Synthesis of N6,N6,1-Trimethylindazole-6,7-Diamine

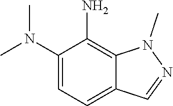

To a stirred solution of N,N,1-trimethyl-7-nitroindazol-6-amine (450 mg, 2.0 mmol, 1.0 eq.) in EtOAc (10 mL) was added Pd/C (43 mg, 0.41 mmol, 0.2 eq.) at room temperature under hydrogen. The resulting reaction mixture was filtered; the solid washed with EtOAc (3×100 mL), and the filtrate was concentrated under reduced pressure. The resulting residue was purified by reversed phase flash chromatography with the following conditions: column: C18 silica gel; mobile phase: MeCN in Water (0.1% TFA), 10% to 30% gradient in 10 min; detector: UV 254 nm resulting in the title compound (300 mg, 77%) as a purple oil. (ES, m/z): [M+H]⁺ 191; ¹H NMR (400 MHz, DMSO-d₆) δ 2.60 (s, 6H), 4.26 (s, 3H). 4.90 (s, 2H), 6.96 (d, J=8.4 Hz, 1H), 7.01 (d, J=8.4 Hz, 1H), 7.78 (s, 1H).

### Example 16—Synthesis of 1-Methyl-6-Propyl-1H-Indazol-7-Amine

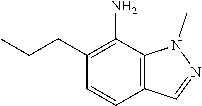

A mixture of 6-cyclopropyl-1-methyl-7-nitroindazole (4 g, 18 mmol, 1 eq.) and Pd/C (2.0 g, 18 mmol, 1 eq.) in EtOAc was stirred for 3 h at room temperature under hydrogen. The resulting reaction mixture was filtered, the solid was washed with EtOAc (2×100 mL), and the filtrate was concentrated under reduced pressure. The residue was purified by reversed-phase flash chromatography with the following conditions: column: C18 silica gel; mobile phase: MeCN in water (0.1% FA), 20% to 50% gradient in 20 min; detector: UV 254 nm. The resulting mixture was concentrated under reduced pressure. The title compound was obtained (150 mg, 4%). (ES, m/z): [M+H]⁺ 190; ¹H NMR (400 MHz, DMSO-d₆) δ 0.94 (t, J=7.6 Hz, 3H), 1.50-1.60 (m, 2H), 2.53-2.63 (m, 2H), 4.29 (s, 3H), 6.75 (d, J=8.0 Hz, 1H), 6.92 (d, J=8.0 Hz, 1H), 7.75 (s, 1H).

### Example 17—Synthesis of 5-Bromo-3-Iodo-4-Nitro-1H-Indazole

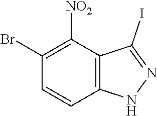 [SMILES: O=[N+]([O-])c1c(Br)ccc2[nH]nc(I)c12]

A mixture of 5-bromo-4-nitro-1H-indazole (4.0 g, 17 mmol, 1 equiv) and NIS (7.4 g, 33 mmol, 2 equiv) in DMF (50 mL) was stirred for 2 h at room temperature under nitrogen. The resulting reaction mixture was diluted with water (200 mL) and extracted with EtOAc (3×50 mL). The combined organic layers were washed with brine (200 mL), dried over anhydrous Na₂SO₄, and concentrated under reduced pressure. The residue was purified by silica gel column chromatography, eluted with PE:EA=30:1 to afford the title compound (2.5 g, 33%) as a yellow solid. (ES, m/z): [M+H]⁺ 368/370.

### Example 18—Synthesis of 5-Bromo-3-Methyl-4-Nitro-1H-Indazole

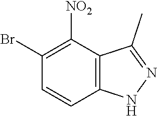

To a stirred solution of 5-bromo-3-iodo-4-nitro-1H-indazole (500 mg, 1.4 mmol, 1 equiv) and Pd(dppf)Cl₂ (99 mg, 0.14 mmol, 0.1 equiv) in dioxane (10 mL) was added dimethylzinc (1.2 mL, 1.2 mmol, 0.9 equiv) dropwise at room temperature under nitrogen, and the resulting reaction mixture was stirred for 5 h at 60° C. The mixture was acidified with 2 M HCl, and then diluted with water (20 mL) and extracted with EtOAc (3×10 mL). The combined organic layers were washed with brine (1×50 mL), dried over anhydrous Na₂SO₄, and concentrated under reduced pressure. The residue was purified by reverse flash chromatography with the following conditions: column: C18 silica gel; mobile phase: MeCN in water (0.1% TFA), 0% to 100% gradient in 30 min; detector: UV 254 nm to afford the title compound (50 mg, 14%) as a yellow solid. (ES, m/z): [M+H]⁺ 256/258.

### Example 19—Synthesis of 3-Methyl-4-Nitro-5-Vinyl-1H-Indazole

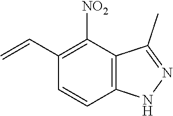

A solution of 5-bromo-3-methyl-4-nitro-1H-indazole (1.9 g, 7.4 mmol, 1 equiv) in the mixture of 1,4-dioxane (16 mL) and H₂O (4 mL), was treated with potassium vinyltrifluoroborate (1.1 g, 8.2 mmol, 1.1 equiv), Pd(dppf)Cl₂ (0.5 g, 0.7 mmol, 0.1 equiv) and TEA (2.6 mL, 19 mmol, 2.5 equiv). The resulting mixture was stirred for additional 2 h at 100° C. The resulting mixture was then diluted with water (50 mL) and extracted with EtOAc (3×30 mL). The combined organic layers were washed with water (2×50 mL), dried over anhydrous Na₂SO₄, and concentrated under reduced pressure. The residue was purified by reverse flash chromatography with the following conditions: column: C18 silica gel; mobile phase: MeCN in water (0.1% TFA), 0% to 100% gradient in 30 min; detector: UV 254 nm to afford the title compound (630 mg, 42%) as a yellow solid. (ES, m/z): [M+H]⁺ 204.

### Example 20—Synthesis of 5-Ethyl-3-Methyl-1H-Indazol-4-Amine

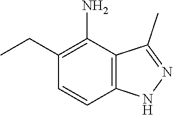

To a stirred solution 5-ethenyl-3-methyl-4-nitro-1H-indazole (300 mg, 1.5 mmol, 1 equiv) in MeOH (10 mL) was added Pd/C (30 mg, 0.3 mmol, 0.2 equiv) at room temperature under nitrogen. The resulting reaction mixture was stirred for 2 h at room temperature under hydrogen, and then filtered. The solids were washed with MeOH (3×10 mL), and the filtrate was concentrated under reduced pressure. The residue was purified by reverse flash chromatography with the following conditions: column: C18 silica gel; mobile phase: MeCN in water (0.1% TFA), 0% to 100% gradient in 40 min; detector: UV 254 nm to afford the title compound (200 mg, 77%) as a yellow oil. (ES, m/z): [M+H]⁺ 176.

### Example 21—Synthesis of 1,3-Dimethyl-4-Nitro-5-Vinyl-1H-Indazole

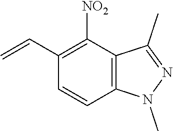 [SMILES: C=Cc1ccc2c(c(C)nn2C)c1[N+](=O)[O-]]

To a stirred mixture of 5-ethenyl-3-methyl-4-nitro-1H-indazole (310 mg, 1.5 mmol, 1 equiv) and Cs₂CO₃ (994 mg, 3 mmol, 2 equiv) in DMF (3 mL) was added CH₃I (0.19 mL, 3 mmol, 2 equiv). The resulting reaction mixture was stirred for additional 2 h at room temperature and then was diluted with water (10 mL) and extracted with EtOAc (3×5 mL). The combined organic layers were washed with water (2×10 mL), dried over anhydrous Na₂SO₄, and concentrated under reduced pressure. The residue was purified by reverse flash chromatography with the following conditions: column: C18 silica gel; mobile phase: MeCN in water (0.1% TFA), 0% to 100% gradient in 30 min; detector: UV 254 nm to afford the title compound (170 mg, 51%) as a yellow oil. (ES, m/z): [M+H]⁺ 218.

### Example 22—Synthesis of 5-Ethyl-1,3-Dimethyl-1H-Indazol-4-Amine

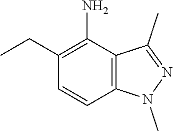

A mixture of 5-ethenyl-1,3-dimethyl-4-nitroindazole (200 mg, 0.9 mmol, 1 equiv) and Pd/C (98 mg, 0.9 mmol, 1 equiv) in MeOH (5 mL) was stirred for 2 h at room temperature under hydrogen. The resulting reaction mixture was filtered and the solids were washed with MeOH (3×10 mL). The filtrate was concentrated under reduced pressure, and the residue was purified by reverse flash chromatography with the following conditions: column: C18 silica gel; mobile phase: MeCN in water (0.1% TFA), 0% to 100% gradient in 30 min; detector: UV 254 nm to afford the title compound (180 mg, 88%) as a yellow oil. (ES, m/z): [M+H]⁺ 218.

### Example 23—Synthesis of 6-Chloro-N-(6-Ethyl-1-Methyl-1H-Indazol-7-yl)Pyridazine-3-Sulfonamide

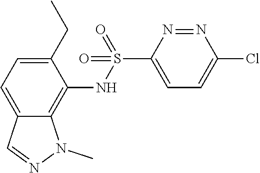 [SMILES: CCc1ccc2cnn(C)c2c1NS(=O)(=O)c1ccc(Cl)nn1]

To a stirred solution of 6-chloropyridazine-3-sulfonyl chloride (100 mg, 0.47 mmol, 1 eq.) in pyridine (2 mL) was added 6-ethyl-1-methylindazol-7-amine (165 mg, 0.94 mmol, 2 eq.). The resulting mixture was stirred at room temperature for 1 h. The resulting reaction mixture was diluted with H₂O (50 mL) and extracted with DCM (3×40 mL). The combined organic layers were washed with brine (3×40 mL), dried over anhydrous Na₂SO₄, and concentrated under reduced pressure. The crude product was purified by reverse phase flash with the following conditions: column: C18 silica gel; mobile phase: MeCN in water, 10% to 80% gradient in 10 min; detector: UV 254 nm to afford the title compound (50 mg, 30%) as a brown oil. (ES, m/z): [M+H]⁺ 352.

### Example 24—Synthesis of 6-Chloro-N-(6-Methoxy-1-Methyl-1H-Indazol-7-yl)Pyridine-3-Sulfonamide

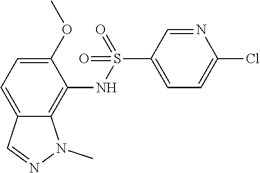 [SMILES: COc1ccc2cnn(C)c2c1NS(=O)(=O)c1ccc(Cl)nc1]

A mixture of 6-methoxy-1-methylindazol-7-amine (300 mg, 1.7 mmol, 1 eq.) and 6-chloropyridine-3-sulfonyl chloride (897 mg, 4.2 mmol, 2.5 eq.) in pyridine (4 mL) was stirred for 2 h at 80° C. The resulting reaction mixture was extracted with DCM (3×100 mL), and the combined organic layers were washed with brine (3×100 mL), dried over anhydrous Na₂SO₄, and concentrated under reduced pressure. The crude product was purified by reverse phase flash with the following conditions: column: C18 silica gel; mobile phase: MeCN in water, 10% to 80% gradient in 10 min; detector: UV 254 nm to afford the title compound (182 mg, 30%) as a brown solid. (ES, m/z): [M+H]⁺ 352.

### Example 25—Synthesis of 2-Chloro-N-(6-Ethyl-1-Methyl-1H-Indazol-7-yl)Pyrimidine-5-Sulfonamide

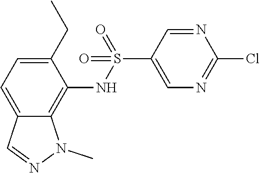 [SMILES: CCc1ccc2cnn(C)c2c1NS(=O)(=O)c1cnc(Cl)nc1]

To a stirred mixture of 2-chloropyrimidine-5-sulfonyl chloride (500 mg, 2.3 mmol, 1 eq.) and ethyl-1-methylindazol-7-amine (0.41 g, 2.3 mmol, 1 eq.) in THE (30 mL) was added Cs₂CO₃ (1.5 g, 4.7 mmol, 2 eq.) in portions at room temperature under nitrogen. The resulting mixture was stirred for 16 h at rt, and then washed with water (20 mL). The aqueous layer was extracted with EtOAc (3×20 mL), dried over anhydrous Na₂SO₄, and concentrated under reduced pressure. The residue was purified by reverse flash chromatography with the following conditions: column: C18 silica gel; mobile phase: MeCN in water, 0% to 100% gradient in 30 min; detector: UV 254 nm to afford the title compound (70 mg, 8.5%) as a purple solid. (ES, m/z): [M+H]⁺ 352.

### Example 26—Synthesis of 5-Bromo-N-(6-Ethyl-1-Methyl-1H-Indazol-7-yl)Pyridine-2-Sulfonamide

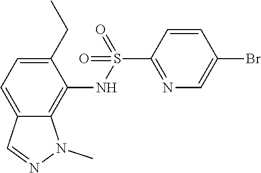 [SMILES: CCc1ccc2cnn(C)c2c1NS(=O)(=O)c1ccc(Br)cn1]

A solution of 5-bromopyridine-2-sulfonyl chloride (281 mg, 1.1 mmol, 1 eq.) and 6-ethyl-1-methylindazol-7-amine (192 mg, 1.1 mmol, 1 eq.) in pyridine (3 mL) was stirred for 16 h at rt. The resulting reaction mixture was washed with water (20 mL), and the aqueous layer was extracted with EtOAc (3×20 mL), dried over anhydrous Na₂SO₄, and concentrated under reduced pressure. The residue was purified by reverse flash chromatography with the following conditions: column: C18 silica gel; mobile phase: MeCN in water, 0% to 100% gradient in 30 min; detector: UV 254 nm to afford the title compound (320 mg, 74%) as a yellow solid. (ES, m/z): [M+H]⁺ 352.

### Example 27—Synthesis of 6-(4-(Trifluoromethyl)-1H-Pyrazol-1-yl)Pyridine-3-Sulfonyl Chloride

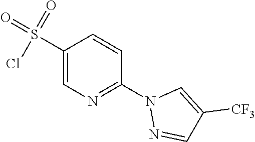 [SMILES: O=S(=O)(Cl)c1ccc(-n2cc(C(F)(F)F)cn2)nc1]

A solution of 4-(pyrazol-1-yl)-2-(trifluoromethyl)pyridine (13 g, 61 mmol, 1.0 eq.) and chlorosulfonic acid (100 mL) was stirred for 16 h at 100° C. The reaction was then quenched with HCl/ice, and the resulting mixture was extracted with DCM (2×120 mL). The combined organic layers were washed with brine (2×40 mL), dried over anhydrous Na₂SO₄, and concentrated under reduced pressure to afford the title sulfonyl chloride (19 g, 100%) as a brown yellow solid. (ES, m/z): [M+H]⁺ 312; ¹H NMR (DMSO-d₆, 400 MHz) δ 7.92 (dd, J=5.6, 2.1 Hz, 1H), 8.15 (d, J=2.1 Hz, 1H), 8.78 (s, 1H), 8.28 (s, 1H), 8.93 (d, J=5.6 Hz, 1H).

### Example 28—Synthesis of 5-Bromo-N-(6-Ethyl-1-Methyl-1H-Indazol-7-yl)Pyridine-2-Sulfonamiden-(6-Ethyl-1-Methyl-1H-Indazol-7-yl)-5-(4-(Trifluoromethyl)-1H-Pyrazol-1-yl)Pyridine-2-Sulfonamide (I-7)

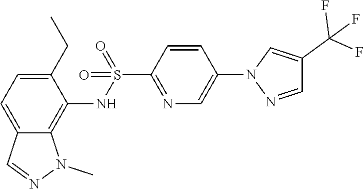 [SMILES: CCc1ccc2cnn(C)c2c1NS(=O)(=O)c1ccc(-n2cc(C(F)(F)F)cn2)cn1]

The title compound was obtained using general procedure B as a white solid (117 mg, 65%). (ES, m/z): [M+1]=451; ¹H NMR (400 MHz, DMSO-d₆) δ 0.78 (t, J=7.6 Hz, 3H), 2.14 (s, 2H), 4.25 (s, 3H), 6.94 (d, J=8.4 Hz, 1H), 7.62 (d, J=8.4 Hz, 1H), 7.95 (d, J=8.4 Hz, 1H), 8.00 (s, 1H), 8.41 (s, 1H), 8.53 (dd, J=8.4 Hz, 6.0 Hz, 1H), 9.39 (d, J=2.4 Hz, 1H), 9.48 (s, 1H), 10.42 (s, 1H).

### Example 29—Synthesis of N-(6-Ethyl-1-Methyl-1H-Indazol-7-yl)-2-(4-(Trifluoromethyl)-1H-Pyrazol-1-yl)Pyrimidine-5-Sulfonamide (I-8)

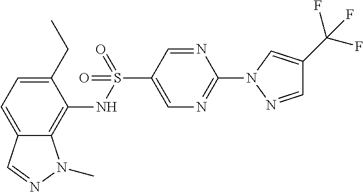 [SMILES: CCc1ccc2cnn(C)c2c1NS(=O)(=O)c1cnc(-n2cc(C(F)(F)F)cn2)nc1]

To a stirred mixture of 2-chloro-N-(6-ethyl-1-methylindazol-7-yl)pyrimidine-5-sulfonamide (160 mg, 0.5 mmol, 1 eq.) and 4-(trifluoromethyl)-1H-pyrazole (93 mg, 0.7 mmol, 1.5 eq.) in THE (10 mL) was added Cs₂CO₃ (445 mg, 1.4 mmol, 3 eq.) at room temperature under a nitrogen atmosphere. The resulting reaction mixture was stirred for 2 h at 60° C. under a nitrogen atmosphere. The reaction was monitored by LCMS. The reaction mixture was diluted with water (20 mL). The aqueous layer was extracted with EtOAc (3×20 mL) and the organiclayer was concentrated under vacuum. The residue was purified by reverse flash chromatography with the following conditions: column: C18 silica gel; mobile phase: MeCN in water, 0% to 100% gradient in 10 min; detector: UV 254 nm. The resulting mixture was concentrated under vacuum to afford the title compound (117 mg, 5%) as an off-white solid. (ES, m/z): [M+H]⁺ 452; ¹H NMR (400 MHz, CDCl₃) δ 1.21 (t, J=7.6 Hz, 3H), 2.70 (q, J=7.6 Hz, 2H), 3.99 (s, 3H), 7.14 (d, J=8.3 Hz, 1H), 7.36 (s, 1H), 7.71 (d, J=8.3 Hz, 1H), 7.96 (d, J=6.6 Hz, 2H), 8.42 (s, 1H), 8.80 (d, J=3.1 Hz, 1H), 9.00 (d, J=3.1 Hz, 1H).

### Example 30—Synthesis of N-(6-Ethyl-1-Methyl-1H-Indazol-7-yl)-6-(4-(Trifluoromethyl)-1H-Pyrazol-1-yl)Pyridazine-3-Sulfonamide (I-9)

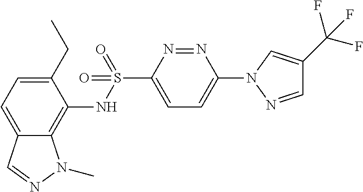 [SMILES: CCc1ccc2cnn(C)c2c1NS(=O)(=O)c1ccc(-n2cc(C(F)(F)F)cn2)nn1]

The title compound was obtained using general procedure B as a brown solid (12 mg, 23%). (ES, m/z): [M+H]⁺ 452; ¹H NMR (400 MHz, DMSO-d₆) δ 0.78 (t, J=7.5, 7.5 Hz, 3H), 1.23 (s, 1H), 2.17 (q, J=7.5, 7.6, 7.6 Hz, 2H), 4.23 (s, 3H), 6.87 (d, J=8.3 Hz, 1H), 7.91 (s, 1H), 8.15 (d, J=9.1 Hz, 1H), 8.38 (d, J=9.1 Hz, 1H), 8.50 (s, 1H), 9.59 (s, 1H), 10.89 (s, 1H).

### Example 31—Synthesis of N-(6-Methoxy-1-Methylindazol-7-yl)-6-[4-(Trifluoromethyl) Pyrazol-1-yl]pyridine-3-Sulfonamide (I-10)

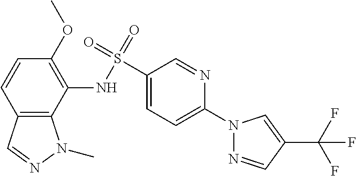 [SMILES: COc1ccc2cnn(C)c2c1NS(=O)(=O)c1ccc(-n2cc(C(F)(F)F)cn2)nc1]

The title compound was obtained using general procedure A as a white solid (43 mg, 11%). (ES, m/z): [M+H]⁺ 453; ¹H NMR (400 MHz, DMSO-d₆) δ 3.19 (s, 3H), 4.28 (s, 3H), 6.83 (d, J=8.9 Hz, 1H), 7.69 (d, J=8.8 Hz, 1H), 8.00 (s, 1H), 8.18 (dd, J=8.6, 0.8 Hz, 1H), 8.29 (dd, J=8.6, 2.4 Hz, 1H), 8.41 (s, 1H), 8.68 (dd, J=2.4, 0.7 Hz, 1H), 9.33 (t, J=1.1 Hz, 1H), 10.12 (s, 1H).

### Example 32—Synthesis of 6-(4-Cyclopropyl-1H-Pyrazol-1-yl)-N-(6-Methoxy-1-Methyl-1H-Indazol-7-yl)Pyridine-3-Sulfonamide (I-11)

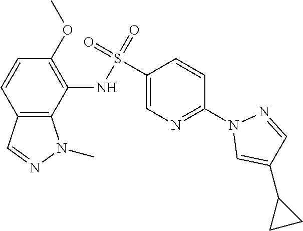 [SMILES: COc1ccc2cnn(C)c2c1NS(=O)(=O)c1ccc(-n2cc(C3CC3)cn2)nc1]

The title compound was obtained using general procedure B as a white solid (20 mg, 11%). ES, m/z): [M+H]⁺ 425; ¹H NMR (400 MHz, DMSO-d₆) δ 0.52-0.74 (m, 2H), 0.76-0.99 (m, 2H), 1.82 (tt, J=5.1, 5.1, 8.4, 8.4 Hz, 1H), 3.17 (s, 3H), 4.27 (s, 3H), 6.83 (d, J=8.9 Hz, 1H), 7.68 (d, J=8.8 Hz, 1H), 7.75 (d, J=0.8 Hz, 1H), 7.93-8.07 (m, 2H), 8.15 (dd, J=2.4, 8.7 Hz, 1H), 8.45 (s, 1H), 8.56 (d, J=2.3 Hz, 1H), 10.01 (s, 1H).

### Example 33—Synthesis of N-(6-Cyclopropyl-1-Methyl-1H-Indazol-7-yl)-1-(5-(Trifluoromethyl)Pyridin-2-yl)-1H-Pyrazole-4-Sulfonamide (I-12)

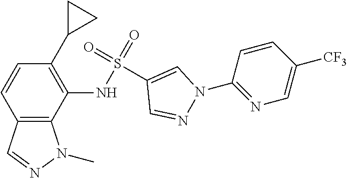 [SMILES: Cn1ncc2ccc(C3CC3)c(NS(=O)(=O)c3cnn(-c4ccc(C(F)(F)F)cn4)c3)c21]

The title compound was obtained using general procedure A as a white solid (36 mg, 15%). (ES, m/z): [M+H]⁺ 463; ¹H NMR (400 MHz, DMSO-d₆) δ 0.30-0.60 (m, 4H), 1.55-1.70 (m, 1H), 4.31 (s, 3H), 6.35 (d, J=8.4 Hz, 1H), 7.59 (d, J=8.4 Hz, 1H), 7.99 (s, 1H), 8.17 (d, J=8.8 Hz, 1H), 8.26 (dd, J=8.8 Hz, 2.4 Hz, 1H), 8.41 (s, 1H), 8.61 (d, J=2.4 Hz, 1H), 9.31 (s, 1H), 10.51 (s, 1H).

### Example 34—Synthesis of 6-(4-Chloro-1H-Pyrazol-1-yl)-N-(6-Methoxy-1-Methyl-1H-Indazol-7-yl)Pyridine-3-Sulfonamide (I-13)

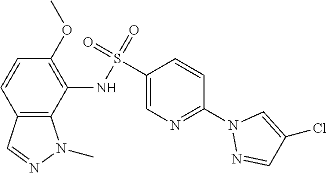 [SMILES: COc1ccc2cnn(C)c2c1NS(=O)(=O)c1ccc(-n2cc(Cl)cn2)nc1]

The title compound was obtained using general procedure B as an off-white solid (49.0 mg, 45%). (ES, m/z): [M+H]⁺ 419; ¹H NMR (400 MHz, DMSO-d₆) δ 3.18 (s, 3H), 4.28 (s, 3H), 6.83 (d, J=8.9 Hz, 1H), 7.69 (d, J=8.8 Hz, 1H), 8.00 (s, 1H), 8.02-8.15 (m, 2H), 8.24 (dd, J=2.4, 8.7 Hz, 1H), 8.57-8.79 (m, 1H), 8.88-9.04 (m, 1H), 10.10 (s, 1H).

### Example 35—Synthesis of N-(6-Methoxy-1-Methyl-1H-Indazol-7-yl)-6-(4-Methyl-1H-Pyrazol-1-yl)Pyridine-3-Sulfonamide (I-14)

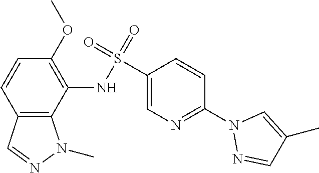 [SMILES: COc1ccc2cnn(C)c2c1NS(=O)(=O)c1ccc(-n2cc(C)cn2)nc1]

The title compound was obtained using general orocedure B as an off-white solid (21.6 mg, 24%). (ES, m/z): [M+H]⁺ 399; ¹H NMR (400 MHz, CDCl₃) δ 2.17 (s, 3H), 3.27 (s, 3H), 4.43 (s, 3H), 6.48 (s, 1H), 6.59 (d, J=8.8 Hz, 1H), 7.61 (d, J=8.6 Hz, 2H), 7.88 (dd, J=2.3, 8.7 Hz, 1H), 7.89-7.96 (m, 2H), 8.30-8.35 (m, 1H), 8.52-8.58 (m, 1H).

### Example 36—Synthesis of N-(6-Methoxy-1-Methyl-1H-Indazol-7-yl)-6-(1H-Pyrazol-1-yl) Pyridine-3-Sulfonamide (I-15)

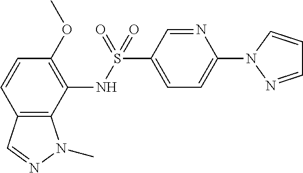 [SMILES: COc1ccc2cnn(C)c2c1NS(=O)(=O)c1ccc(-n2cccn2)nc1]

The title compound was obtained using general procedure B as an off-white solid (24.9 mg, 28%). (ES, m/z): [M+H]⁺ 385; H NMR (400 MHz, DMSO-d₆) δ 3.18 (s, 3H), 4.28 (s, 3H), 6.66 (dd, J=1.6, 2.7 Hz, 1H), 6.83 (d, J=8.9 Hz, 1H), 7.68 (d, J=8.8 Hz, 1H), 8.00 (s, 1H), 8.11 (d, J=8.7 Hz, 1H), 8.20 (d, J=2.4 Hz, 1H), 8.22 (d, J=2.4 Hz, 1H), 8.62 (d, J=2.3 Hz, 1H), 8.72 (d, J=2.6 Hz, 1H), 10.05 (s, 1H).

### Example 37—Synthesis of N-(6-Methoxy-1-Methylindazol-7-yl)-6-(4-Methylpyrazol-1-yl)Pyridine-3-Sulfonamide (I-16)

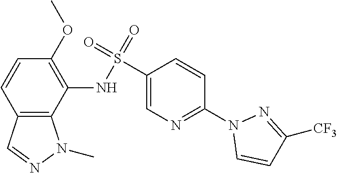 [SMILES: COc1ccc2cnn(C)c2c1NS(=O)(=O)c1ccc(-n2ccc(C(F)(F)F)n2)nc1]

The title compound was obtained using general procedure B as a white solid (0.447 g, 31%). (ES, m/z): [M+H]⁺ 453; ¹H NMR (400 MHz, CDCl₃) δ 3.28 (s, 3H), 4.43 (s, 3H), 6.48 (s, 1H), 6.61 (d, J=8.8 Hz, 1H), 6.76 (d, J=2.8 Hz, 1H), 7.64 (d, J=8.8 Hz, 1H), 8.02-7.94 (m, 2H), 8.09-8.02 (m, 1H), 8.68-8.60 (m, 2H).

### Example 38—Synthesis of Additional Compounds

Compounds in Table 3 below were prepared based on procedures described herein.

TABLE 3

| Compound | | |
|---|---|---|
| No. | Structure | LC-MS and ¹H NMR Data |
| I-1 | 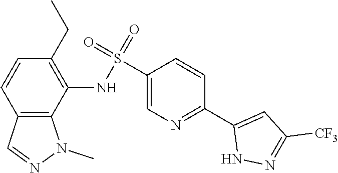 [SMILES: CCc1ccc2cnn(C)c2c1NS(=O)(=O)c1ccc(-c2cc(C(F)(F)F)n[nH]2)nc1] | N/A |
| I-2 | 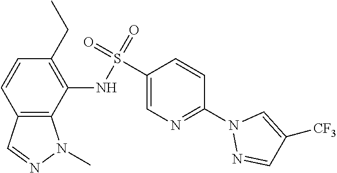 [SMILES: CCc1ccc2cnn(C)c2c1NS(=O)(=O)c1ccc(-n2cc(C(F)(F)F)cn2)nc1] | N/A |
| I-3 | 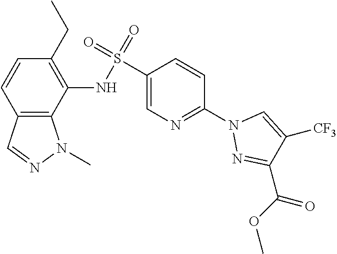 [SMILES: CCc1ccc2cnn(C)c2c1NS(=O)(=O)c1ccc(-n2cc(C(F)(F)F)c(C(=O)OC)n2)nc1] | N/A |
| I-4 | 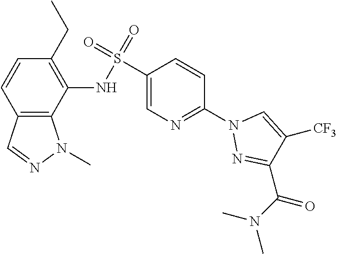 [SMILES: CCc1ccc2cnn(C)c2c1NS(=O)(=O)c1ccc(-n2cc(C(F)(F)F)c(C(=O)N(C)C)n2)nc1] | N/A |
| I-5 | 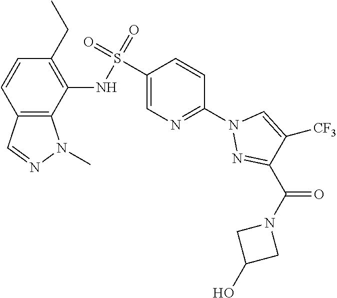 [SMILES: CCc1ccc2cnn(C)c2c1NS(=O)(=O)c1ccc(-n2cc(C(F)(F)F)c(C(=O)N3CC(O)C3)n2)nc1] | N/A |
| I-6 | 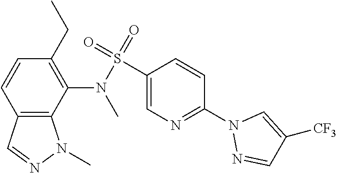 [SMILES: CCc1ccc2cnn(C)c2c1N(C)S(=O)(=O)c1ccc(-n2cc(C(F)(F)F)cn2)nc1] | N/A |
| I-17 | 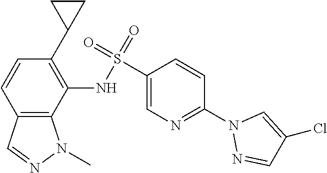 [SMILES: Cn1ncc2ccc(C3CC3)c(NS(=O)(=O)c3ccc(-n4cc(Cl)cn4)nc3)c21] | N/A |
| 1-54 | 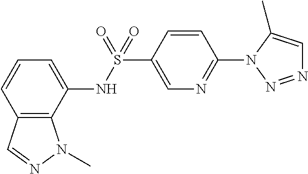 [SMILES: Cc1cnnn1-c1ccc(S(=O)(=O)Nc2cccc3cnn(C)c23)cn1] | N/A |
| I-55 | 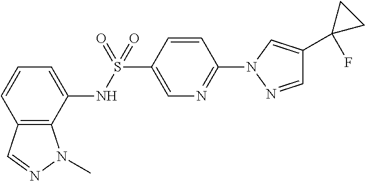 [SMILES: Cn1ncc2cccc(NS(=O)(=O)c3ccc(-n4cc(C5(F)CC5)cn4)nc3)c21] | N/A |
| I-60 | 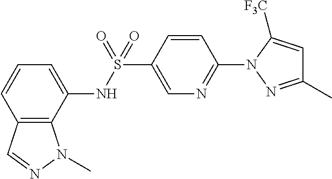 [SMILES: Cc1cc(C(F)(F)F)n(-c2ccc(S(=O)(=O)Nc3cccc4cnn(C)c34)cn2)n1] | N/A |
| I-61 | 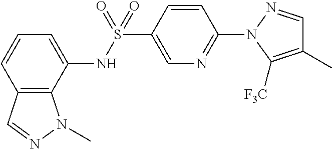 [SMILES: Cc1cnn(-c2ccc(S(=O)(=O)Nc3cccc4cnn(C)c34)cn2)c1C(F)(F)F] | N/A |
| I-78 | 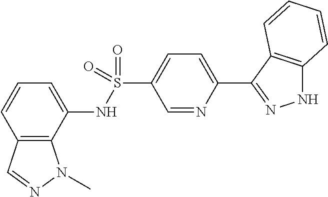 [SMILES: Cn1ncc2cccc(NS(=O)(=O)c3ccc(-c4n[nH]c5ccccc45)nc3)c21] | N/A |
| I-91 | 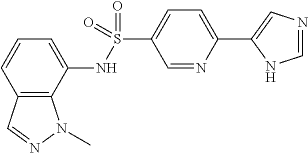 [SMILES: Cn1ncc2cccc(NS(=O)(=O)c3ccc(-c4cnc[nH]4)nc3)c21] | N/A |
| I-92 | 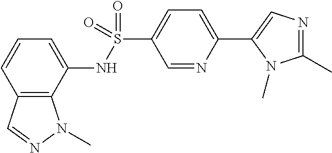 [SMILES: Cc1ncc(-c2ccc(S(=O)(=O)Nc3cccc4cnn(C)c34)cn2)n1C] | N/A |
| I-93 | 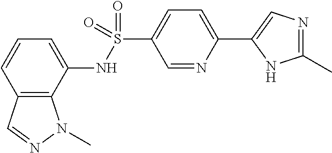 [SMILES: Cc1ncc(-c2ccc(S(=O)(=O)Nc3cccc4cnn(C)c34)cn2)[nH]1] | N/A |
| I-94 | 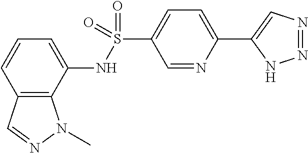 [SMILES: Cn1ncc2cccc(NS(=O)(=O)c3ccc(-c4cnn[nH]4)nc3)c21] | N/A |
| I-95 | 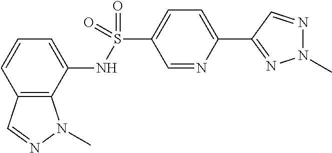 [SMILES: Cn1ncc(-c2ccc(S(=O)(=O)Nc3cccc4cnn(C)c34)cn2)n1] | N/A |
| I-96 | 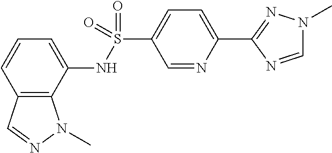 [SMILES: Cn1cnc(-c2ccc(S(=O)(=O)Nc3cccc4cnn(C)c34)cn2)n1] | N/A |
| I-97 | 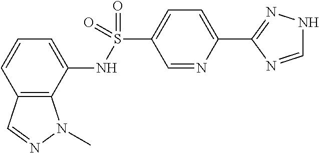 [SMILES: Cn1ncc2cccc(NS(=O)(=O)c3ccc(-c4nc[nH]n4)nc3)c21] | N/A |
| I-99 | 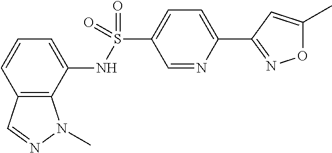 [SMILES: Cc1cc(-c2ccc(S(=O)(=O)Nc3cccc4cnn(C)c34)cn2)no1] | N/A |
| I-100 | 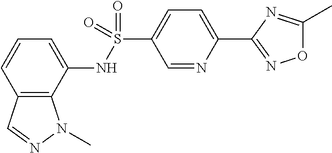 [SMILES: Cc1nc(-c2ccc(S(=O)(=O)Nc3cccc4cnn(C)c34)cn2)no1] | N/A |
| I-102 | 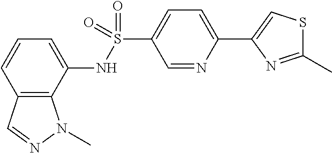 [SMILES: Cc1nc(-c2ccc(S(=O)(=O)Nc3cccc4cnn(C)c34)cn2)cs1] | N/A |
| I-274 | 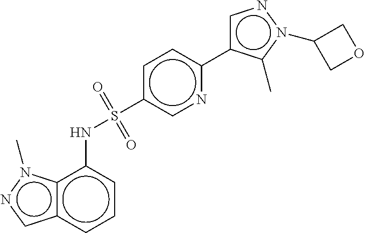 | LC-MS (ES, m/z): [M + H]⁺ = 425. ¹H NMR (400 MHz, methanol-d₄): δ 2.62 (s, 3H), 4.36 (s, 3H), 5.01-5.09 (m, 2H), 5.14 (t, J = 6.4 Hz, 2H), 5.72 (t, J = 6.8 Hz, 1H), 6.54 (dd, J = 1.2, 7.2 Hz, 1H), 6.92 (dd, J = 5.2, 8.8 Hz, 1H), 7.69 (d, J = 8.0 Hz, 1H), 7.77 (dd, J = 0.8, 8.4 Hz, 1H), 7.96 (dd, J = 2.4, 8.4 Hz, 1H), 8.03 (s, 1H), 8.13 (s, 1H), 8.71 (s, 1H). |
| I-275 | 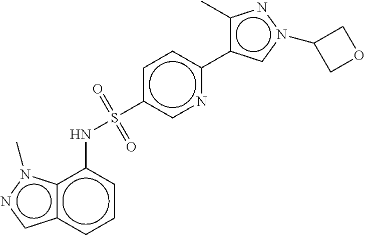 | LC-MS (ES, m/z): [M + H]⁺ = 425. ¹H NMR (400 MHz, methanol-d₄): δ 2.59 (s, 3H), 4.36 (s, 3H), 5.06 (d, J = 6.8 Hz, 4H), 5.55 (p, J = 6.8 Hz, 1H), 6.54 (dd, J = 1.2, 7.2 Hz, 1H), 6.90 (t, J = 7.6 Hz, 1H), 7.69-7.72 (m, 2H), 7.94 (dd, J = 2.4, 8.5 Hz, 1H), 8.03 (s, 1H), 8.35 (s, 1H), 8.72 (s, 1H). |
| I-277 | 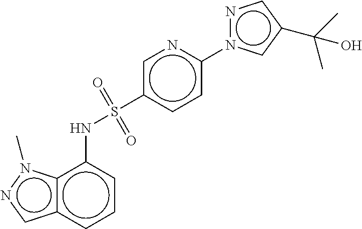 | LC-MS (ES, m/z): [M + H]⁺ = 413.20. ¹H NMR (400 MHz, DMSO-d₆): δ 1.47 (s, 6H), 4.29 (s, 3H), 5.08 (s, 1H), 6.51 (d, J = 7.2 Hz, 1H), 6.94 (t, J = 7.6 Hz, 1H), 7.70 (d, J = 8.0 Hz, 1H), 7.89 (s, 1H), 8.07(s, 1H), 8.09 (d, J = 3.2 Hz, 1H), 8.19 (dd, J = 2.0, 8.4 Hz, 1H), 8.49 (s, 1H), 8.57 (d, J = 2.0 Hz, 1H), 10.26 (s, 1H). |
| I-279 | 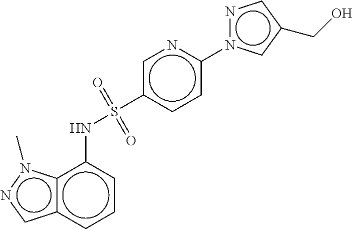 | LC-MS (ES, m/z): [M + H]⁺ = 385. ¹H NMR (400 MHz, DMSO-d₆): δ 4.28 (s, 3H), 4.47 (d, J = 5.4 Hz, 2H), 5.09 (t, J = 5.4 Hz, 1H), 6.52 (d, J = 7.4 Hz, 1H), 6.94 (t, J = 7.8 Hz, 1H), 7.70 (d, J = 8.0 Hz, 1H), 7.87 (s, 1H), 8.09 (d, J = 7.8 Hz, 2H), 8.20 (dd, J = 8.6, 2.4 Hz, 1H), 8.53-8.62 (m, 2H), 10.27 (s, 1H). |
| I-282 | 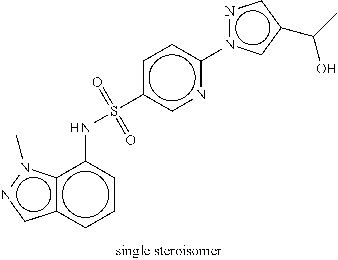 | LC-MS (ES, m/z): [M + H]⁺ = 399. ¹H NMR (400 MHz, DMSO-d₆): δ 1.36 (d, J = 6.6 Hz, 3H), 4.40 (s, 3H), 4.74 (d, J = 6.6 Hz, 1H), 5.06 (s, 1H), 6.64 (t, J = 7.8 Hz, 1H), 6.79 (dd, J = 7.6, 2.4 Hz, 2H), 7.70 (d, J = 15.8 Hz, 2H), 7.83 (d, J = 8.6 Hz, 1H), 8.17 (dd, J = 8.6, 2.4 Hz, 1H), 8.40 (s, 1H), 8.67 (d, J = 2.2 Hz, 1H). |
| I-283 | 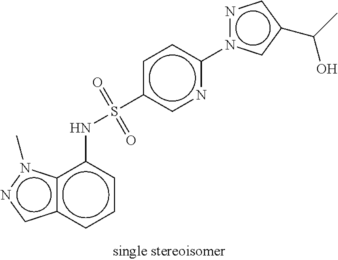 | LC-MS (ES, m/z): [M + H]⁺ = 399. ¹H NMR (400 MHz, DMSO-d₆): δ 1.37 (d, J = 6.6 Hz, 3H), 4.40 (s, 3H), 4.74 (d, J = 6.6 Hz, 1H), 5.06 (s, 1H), 6.60-6.68 (m, 1H), 6.76-6.83 (m, 2H), 7.70 (d, J = 15.8 Hz, 2H), 7.83 (d, J = 8.6 Hz, 1H), 8.17 (dd, J = 8.6, 2.4 Hz, 1H), 8.40 (s, 1H), 8.67 (d, J = 2.2 Hz, 1H). |
| I-287 | 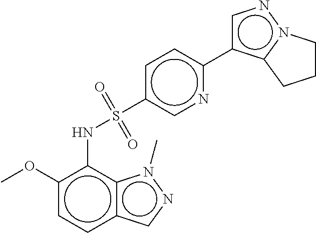 | LC-MS (ES, m/z): [M + 1]⁺ = 425. ¹H NMR (400 MHz, DMSO-d₆): δ 2.63 (p, J = 7.6 Hz, 2H), 3.14 (s, 3H), 3.15 (t, J = 7.6 Hz, 2H), 4.14 (t, J = 7.2 Hz, 2H), 4.27 (s, 3H), 6.81 (d, J = 8.8 Hz, 1H), 7.65 (d, J = 8.8 Hz, 1H), 7.70 (dd, J = 8.4, 0.8 Hz, 1H), 7.90 (dd, J = 8.4, 2.4 Hz, 1H), 7.98 (s, 1H), 8.12 (s, 1H), 8.64 (dd, J = 2.4, 0.8 Hz, 1H), 9.84 (s, 1H). |
| I-288 | 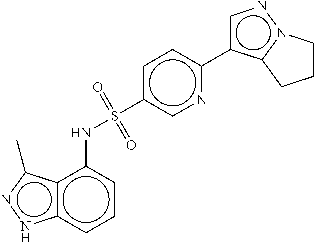 | LC-MS (ES, m/z): [M + 1]⁺ = 395. ¹H NMR (400 MHz, DMSO-d₆): δ 2.54 (s, 3H), 2.62 (p, J = 7.2 Hz, 2H), 3.12 (t, J = 7.2 Hz, 2H), 4.12 (t, J = 7.2 Hz, 2H), 6.41 (d, J = 7.2 Hz, 1H), 7.15 (t, J = 7.6 Hz, 1H), 7.34 (d, J = 8.0 Hz, 1H), 7.70 (d, J = 8.4 Hz, 1H), 7.93 (dd, J = 8.4, 2.4 Hz, 1H), 8.11 (s, 1H), 8.66 (d, J = 2.4 Hz, 1H), 10.03 (s, 1H), 12.74 (s, 1H). |
| I-289 | 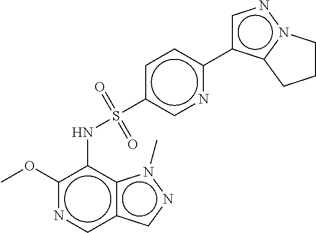 | LC-MS (ES, m/z): [M + 1]⁺ = 426. ¹H NMR (400 MHz, DMSO-d₆): δ 2.59-2.67 (m, 2H). 3.15 (t, J = 7.2 Hz, 2H), 3.25 (s, 3H), 4.14 (t, J = 7.2 Hz, 2H), 4.24 (s, 3H), 7.71 (d, J = 8.4 Hz, 1H), 7.90 (dd, J = 8.4, 2.4 Hz, 1H), 8.12 (s, 1H), 8.22 (s, 1H), 8.62 (s, 1H), 8.64 (d, J = 1.6 Hz, 1H), 9.92 (s, 1H) |
| I-295 | 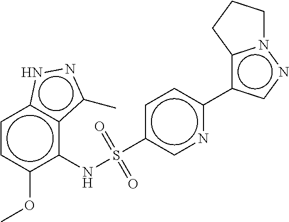 | LC-MS (ES, m/z): [M + 1]⁺ = 425. ¹H NMR (400 MHz, DMSO-d₆): δ 2.61-2.63 (m, 2H), 2.64 (s, 3H), 3.12 (s, 3H), 3.14-3.16 (m, 2H), 4.13 (t, J = 7.2 Hz, 2H), 7.01 (d, J = 9.2 Hz, 1H), 7.35 (d, J = 8.8 Hz, 1H), 7.69 (d, J = 8.4 Hz, 1H), 7.90 (dd, J = 8.4, 2.4 Hz, 1H), 8.11 (s, 1H), 8.63 (d, J = 2.4 Hz, 1H), 9.74 (s, 1H), 12.55 (s, 1H). |
| I-297 | 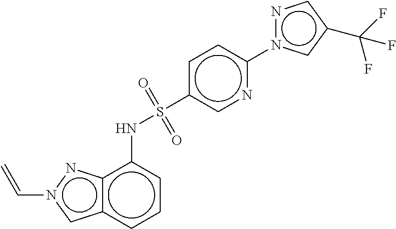 | LC-MS (ES, m/z): [M + 1]⁺ = 435. ¹H NMR (400 MHz, DMSO-d₆): δ 4.87 (d, J = 8.8 Hz, 1H), 5.62 (d, J = 15.4 Hz, 1H), 6.64 (d, J = 7.4 Hz, 1H), 7.04 (t, J = 7.8 Hz, 1H), 7.78 (d, J = 8.0 Hz, 1H), 8.12 (d, J = 8.8 Hz, 1H), 8.23-8.12 (m, 1H), 8.28 (dd, J = 8.8, 2.4 Hz, 1H), 8.42 (s, 1H), 8.37 (s, 1H), 8.65 (d, J = 2.4 Hz, 1H), 9.32 (s, 1H), 10.53 (s, 1H). |
| I-298 | 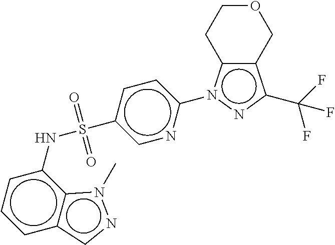 | LC-MS (ES, m/z): [M + H]⁺ = 479. ¹H NMR (400 MHz, DMSO-d₆): δ 3.28 (d, J = 11.2 Hz, 2H), 3.89 (t, J = 5.6 Hz, 2H), 4.30 (s, 3H), 4.73 (s, 2H), 6.54 (d, J = 7.4 Hz, 1H), 6.91 (t, J = 7.8 Hz, 1H), 7.64 (d, J = 8.0 Hz, 1H), 8.07 (s, 1H), 8.13 (d, J = 8.8 Hz, 1H), 8.27 (m, J = 8.8, 2.4 Hz, 1H), 8.66 (d, J = 2.4 Hz, 1H), 10.38 (s, 1H). |
| I-299 | 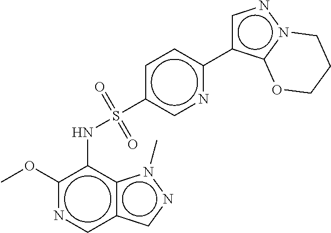 | LC-MS (ES, m/z): [M + H]⁺ = 442. ¹H NMR: (400 MHz, DMSO-d₆): δ 2.15-2.25 (m, 2H), 3.17 (s, 3H), 4.09 (t, J = 6.4 Hz, 2H), 4.16 (s, 3H), 4.45 (t, J = 5.2 Hz, 2H), 7.60 (d, J = 8.4 Hz, 1H), 7.82 (dd, J = 8.6, 2.4 Hz, 1H), 7.87 (s, 1H), 8.15 (s, 1H), 8.52 (d, J = 2.4 Hz, 1H), 8.56 (s, 1H), 9.88 (s, 1H). |
| I-301 | 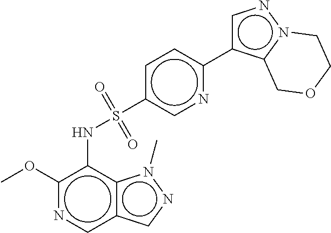 | LC-MS (ES, m/z): [M +1 ]⁺ = 442. ¹H NMR (400 MHz, DMSO-d₆): δ 3.24 (s, 3H), 4.10 (t, J = 5.2 Hz, 2H), 4.20 (t, J = 5.2 Hz, 2H), 4.24 (s, 3H), 5.14 (s, 2H), 7.85 (d, J = 8.4 Hz, 1H), 7.92 (dd, J = 8.4, 2.4 Hz, 1H), 8.22 (s, 2H), 8.64 (s, 1H), 8.66 (d, J = 2.0 Hz, 1H), 10.01 (s, 1H). |
| I-305 | 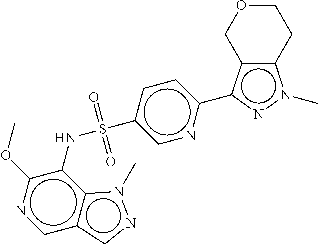 | LC-MS (ES, m/z): [M + H]⁺ = 456. ¹H NMR (400 MHz, DMSO-d₆): δ 2.76 (t, J = 5.6 Hz, 2H), 3.21 (s, 3H), 3.82 (s, 3H), 3.87 (t, J = 8.0 Hz, 2H), 4.24 (s, 3H), 4.86 (s, 2H), 7.99 (dd, J = 8.4, 4.0 Hz, 1H), 8.05 (d, J = 8.0 Hz, 1H), 8.21 (s, 1H), 8.61 (s, 1H), 8.73-8.68 (m, 1H), 10.08 (s, 1H). |
| I-307 | 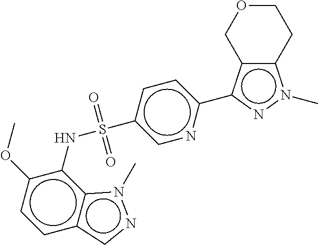 | LC-MS (ES, m/z): [M + 1]⁺ = 455. ¹H NMR (400 MHz, DMSO-d₆): δ 2.76 (t, J = 5.6 Hz, 2H), 3.10 (s, 3H), 3.82 (s, 3H), 3.87 (t, J = 5.6 Hz, 2H), 4.27 (s, 3H), 4.87 (s, 2H), 6.81 (d, J = 8.8 Hz, 1H), 7.66 (d, J = 8.8 Hz, 1H), 8.02-7.95 (m, 2H), 8.06 (d, J = 8.4 Hz, 1H), 8.70 (d, J = 2.2 Hz, 1H), 9.93 (s, 1H). |
| I-308 | 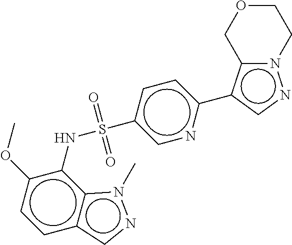 | LC-MS (ES, m/z): [M + H]⁺ = 441. ¹H NMR (400 MHz, chloroform-d): 83.21 (s, 3H), 4.15 (dd, J = 5.8, 4.4 Hz, 2H), 4.27 (t, J = 5.2 Hz, 2H), 4.42 (s, 3H), 5.19 (s, 2H), 6.42 (s, 1H), 6.57 (d, J = 8.8 Hz, 1H), 7.43 (d, J = 8.4 Hz, 1H), 7.60 (d, J = 8.8 Hz, 1H), 7.72 (dd, J = 8.4, 2.4 Hz, 1H), 7.93 (s, 2H), 8.70 (d, J = 2.3 Hz, 1H). |
| I-311 | 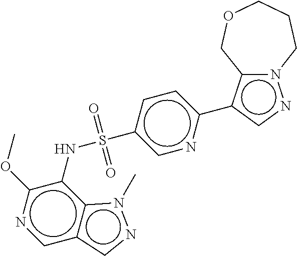 | LC-MS (ES, m/z): [M + 1]⁺ = 456. ¹H NMR (400 MHz, DMSO-d₆): δ 1.90 (s, 2H), 3.24 (s, 3H), 3.99 (t, J = 5.1 Hz, 2H), 4.24 (s, 3H), 4.46-4.53 (m, 2H), 5.21 (s, 2H), 7.86 (d, J = 8.5 Hz, 1H), 7.94 (dd, J = 2.4, 8.5 Hz, 1H), 8.00 (s, 1H), 8.23 (s, 1H), 8.64 (s, 1H), 8.69 (d, J = 2.3 Hz, 1H), 10.06 (s, 1H). |
| I-313 | 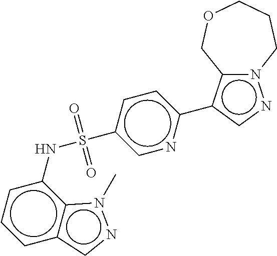 | LC-MS (ES, m/z): [M + 1]⁺ 424. ¹H NMR (400 MHz, DMSO-d₆): δ 1.89 (t, J = 4.4 Hz, 2H), 3.99 (t, J = 4.8 Hz, 2H), 4.29 (s, 3H), 4.49 (t, J = 4.8 Hz, 2H), 5.21 (s, 2H), 6.90 (t, J = 8.0 Hz, 1H), 7.60 (d, J = 6.0 Hz, 1H), 7.88 (d, J = 8.4 Hz, 1H), 7.96-8.01 (m, 1H), 8.05 (s, 1H), 8.14 (s, 1H), 8.70 (d, J = 2.0 Hz, 1H), 10.26 (s, 1H). |
| I-315 | 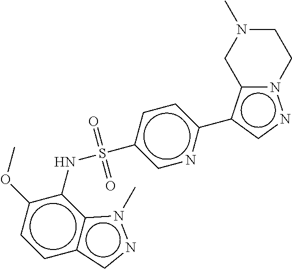 | LC-MS (ES, m/z): [M + H]⁺ = 454. ¹H NMR (400 MHz, DMSO-d₆): δ 1.99 (m, J = 12.4, 4.0 Hz, 2H), 2.35 (m, 2H), 2.54-2.64 (m, 2H), 3.53 (d, J = 12.0 Hz, 3H), 3.80 (s, 3H), 3.98 (s, 3H), 4.30 (s, 3H), 7.05 (d, J = 8.8 Hz, 1H), 7.27 (dd, J = 12.6, 1.0 Hz, 2H), 7.71 (d, J = 8.8 Hz, 1H), 7.91 (s, 1H). |

### Example 39—Synthesis of N-(6-Methoxy-1-Methylindazol-7-yl)-6-[2-Methyl-5-(Trifluoromethyl)Pyrazol-3-yl]pyridine-3-Sulfonamide (I-18)

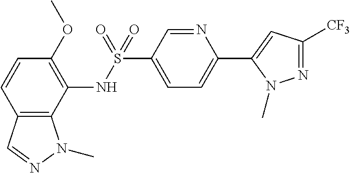 [SMILES: COc1ccc2cnn(C)c2c1NS(=O)(=O)c1ccc(-c2cc(C(F)(F)F)nn2C)nc1]

The title compound was obtained using general procedure D to provide the title compound as a light pink solid (32.5 mg, 24%). (ES, m/z): [M+1]=467; ¹H NMR (400 MHz, DMSO-d₆) δ 3.15 (s, 3H), 4.26 (3, 3H), 4.28 (s, 3H), 6.82 (d, J=8.9 Hz, 1H), 7.53 (s, 1H), 7.68 (d, J=8.9 Hz, 1H), 8.00 (s, 1H), 8.14 (d, J=2.5 Hz, 2H), 8.87 (d, J=1.8 Hz, 1H),10.13 (s, 1H).

### Example 40—Synthesis of N-(6-Methoxy-1-Methylindazol-7-yl)-6-(1-Methylpyrazol-3-yl)Pyridine-3-Sulfonamide (I-19)

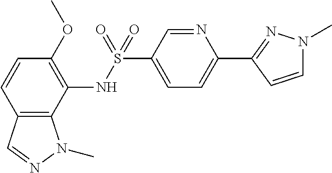 [SMILES: COc1ccc2cnn(C)c2c1NS(=O)(=O)c1ccc(-c2ccn(C)n2)nc1]

The title compound was obtained using general procedure D as a light pink solid (42.7 g, 37%). (ES, m/z): [M+H]⁺ 399; 1H NMR (400 MHz, CDCl₃) δ 3.25 (s, 3H), 3.98 (s, 3H), 4.42 (s, 3H), 6.58 (d, J=8.4 Hz, 2H), 7.44 (dd, J=8.4, 0.8 Hz, 1H), 7.60 (d, J=8.8 Hz, 1H), 7.76 (dd, J=8.4, 2.4 Hz, 1H), 7.93 (s, 1H), 7.99 (d, J=0.8 Hz, 1H), 8.13 (s, 1H), 8.75 (dd, J=2.3, 0.8 Hz, 1H).

### Example 41—Synthesis of N-(6-Methoxy-1-Methylindazol-7-yl)-6-(1-Methylpyrazol-4-yl)Pyridine-3-Sulfonamide (I-20)

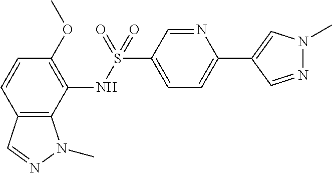 [SMILES: COc1ccc2cnn(C)c2c1NS(=O)(=O)c1ccc(-c2cnn(C)c2)nc1]

The title compound was obtained using general procedure D as a light pink solid (42.8 mg, 37%). (ES, m/z): [M+H]⁺ 399; ¹H NMR (400 MHz, CDCl₃) δ 3.24 (s, 3H), 3.98 (s, 3H), 4.42 (s, 3H), 6.51 (s, 1H), 6.58 (d, J=8.8 Hz, 1H), 7.43 (d, J=8.4 Hz, 1H), 7.60 (d, J=8.8 Hz, 1H), 7.74 (dd, J=8.4, 2.4 Hz, 1H), 7.93 (s, 1H), 7.99 (s, 1H), 8.07 (s, 1H), 8.73 (d, J=2.3 Hz, 1H).

### Example 42—Synthesis of N-(6-Methoxy-1-Methyl-1H-Indazol-7-yl)-6-(1-Methyl-1H-1,2,3-Triazol-4-yl)Pyridine-3-Sulfonamide (I-21)

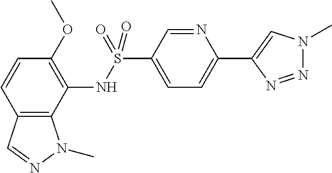 [SMILES: COc1ccc2cnn(C)c2c1NS(=O)(=O)c1ccc(-c2cn(C)nn2)nc1]

The title compound was obtained using general procedure D as a white solid (22.0 mg, 7%). (ES, m/z): [M+H]⁺400; ¹H NMR (400 MHz, DMSO-d₆) δ3.11 (s, 3H), 4.14 (s, 3H), 4.27 (s, 3H), 6.81 (d, J=8.8 Hz, 1H), 7.67 (d, J=8.8 Hz, 1H), 7.99 (s, 1H), 8.11-8.09 (m, 1H), 8.21 (d, J=8.4 Hz, 1H), 8.74 (t, J=2.0 Hz, 2H), 9.94 (s, 1H).

### Example 43—Synthesis of N-(6-Methoxy-1-Methylindazol-7-yl)-6-[5-(Trifluoromethyl)-2H-Pyrazol-3-yl]pyridine-3-Sulfonamide (I-22)

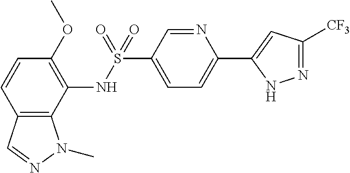 [SMILES: COc1ccc2cnn(C)c2c1NS(=O)(=O)c1ccc(-c2cc(C(F)(F)F)n[nH]2)nc1]

The title compound was obtained using general procedure D as an off white solid (19.2 mg, 15%). (ES, m/z): [M+1]=453, ¹H NMR (400 MHz, DMSO-d₆) δ 3.12 (s, 3H) 4.27 (s, 3H), 6.83 (d, J=8.8 Hz, 1H), 7.60 (dd, J=12 Hz, 4.8 Hz, 1H), 7.67 (d, J=8.8 Hz, 1H), 7.99 (s, 1H), 8.14 (dd, J=8.8 Hz, 2.4 Hz, 2H), 8.79 (s, 1H), 10.07 (s, 1H), 14.63 (s, 1H).

### Example 44—Synthesis of N-(1-Methyl-1H-Indazol-7-yl)-6-(4-(Trifluoromethyl)-1H-Pyrazol-1-yl)Pyridine-3-Sulfonamide (I-23)

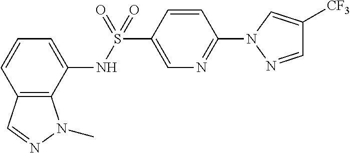 [SMILES: Cn1ncc2cccc(NS(=O)(=O)c3ccc(-n4cc(C(F)(F)F)cn4)nc3)c21]

The title compound was obtained using general procedure B as a white solid (51.8 mg, 18%). (ES, m/z): [M+H]⁺ 423;¹H NMR (400 MHz, DMSO-d₆) δ 4.29 (s, 3H), 6.52 (d, J=7.3 Hz, 1H), 6.93 (t, J=7.7, 7.7 Hz, 1H), 7.69 (d, J=8.1 Hz, 1H), 8.10 (s, 1H), 8.20 (d, J=8.7 Hz, 1H), 8.30 (dd, J=2.4, 8.7 Hz, 1H), 8.43 (s, 1H), 8.67 (d, J=2.3 Hz, 1H), 9.34 (s, 1H), 10.38 (s, 1H).

### Example 45—Synthesis of N-(3-Methyl-1H-Indazol-4-yl)-6-(4-(Trifluoromethyl)-1H-Pyrazol-1-yl)Pyridine-3-Sulfonamide (I-24)

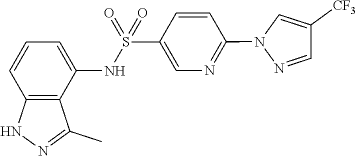 [SMILES: Cc1n[nH]c2cccc(NS(=O)(=O)c3ccc(-n4cc(C(F)(F)F)cn4)nc3)c12]

The title compound was obtained using general procedure A as an off-white solid (11.7 mg, 22%). (ES, m/z): [M+H]⁺ 423; ¹H NMR (400 MHz, DMSO-d₆) δ 2.56 (s, 3H), 6.38 (d, J=7.3 Hz, 1H), 7.14 (t, J=7.8 Hz, 1H), 7.35 (d, J=8.3 Hz, 1H), 8.14-8.20 (m, 1H), 8.29 (dd, J=2.4, 8.7 Hz, 1H), 8.41 (s, 1H), 8.67 (dd, J=0.8, 2.4 Hz, 1H), 9.31 (t, J=1.0 Hz, 1H), 10.27 (s, 1H), 12.77 (s, 1H).

### Example 46—Synthesis of N-(6-Chloro-2-Methyl-2H-Indazol-7-yl)-6-(4-(Trifluoromethyl)-1H-Pyrazol-1-yl)Pyridine-3-Sulfonamide (I-25)

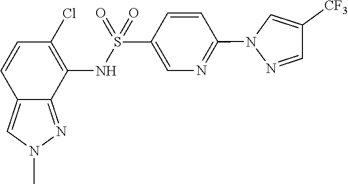 [SMILES: Cn1cc2ccc(Cl)c(NS(=O)(=O)c3ccc(-n4cc(C(F)(F)F)cn4)nc3)c2n1]

The title compound was obtained using general procedure A as a white solid (35.9 mg, 12%). (ES, m/z): [M+H]⁺ 457; ¹H NMR (400 MHz, DMSO-d₆) δ 3.85 (s, 3H), 7.09 (d, J=8.8 Hz, 1H), 7.65 (d, J=8.8 Hz, 1H), 8.13 (d, J=8.6 Hz, 1H), 8.35 (m, 2H), 8.40 (s, 1H), 8.75 (m, 1H), 9.32 (t, J=1.1 Hz, 1H), 10.44 (s, 1H).

### Example 47—Synthesis of N-(6-Ethyl-2-Methylindazol-7-yl)-6-[4-(Trifluoromethyl)Pyrazol-1-yl]pyridine-3-Sulfonamide (I-26)

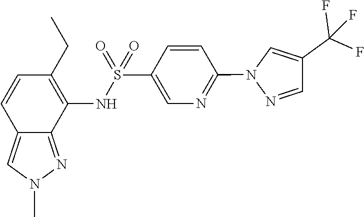 [SMILES: CCc1ccc2cn(C)nc2c1NS(=O)(=O)c1ccc(-n2cc(C(F)(F)F)cn2)nc1]

The title compound was obtained using general procedure A as a white solid (38.2 mg, 5%). (ES, m/z): [M+H]⁺ 451; ¹H NMR (400 MHz, DMSO-d₆) δ 1.20 (t, J=7.5 Hz, 3H), 2.85 (q, J=7.6 Hz, 2H), 3.68 (s, 3H), 6.99 (d, J=8.6 Hz, 1H), 7.55 (d, J=8.5 Hz, 1H), 8.05 (d, J=8.6 Hz, 1H), 8.13 (s, 1H), 8.26 (dd, J=8.6, 2.4 Hz, 1H), 8.40 (s, 1H), 8.67 (d, J=2.3 Hz, 1H), 9.31 (t, J=1.1 Hz, 1H), 10.00 (s, 1H).

### Example 48—Synthesis of N-(6-Methoxy-2-Methylindazol-7-yl)-6-[4-(Trifluoromethyl)Pyrazol-1-yl]pyridine-3-Sulfonamide (I-27)

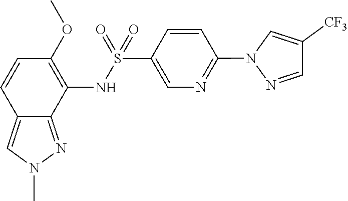 [SMILES: COc1ccc2cn(C)nc2c1NS(=O)(=O)c1ccc(-n2cc(C(F)(F)F)cn2)nc1]

The title compound was obtained using general procedure D as a white solid (35.2 mg, 12%). (ES, m/z): [M+H]⁺ 453; ¹H NMR (400 MHz, DMSO-d₆) δ 3.54 (s, 3H), 3.97 (s, 3H), 6.94 (d, J=9.1 Hz, 1H), 7.62 (d, J=9.0 Hz, 1H), 8.14 (d, J=8.5 Hz, 1H), 8.25 (s, 1H), 8.33-8.42 (m, 2H), 8.77 (d, J=2.3 Hz, 1H), 9.32 (t, J=1.0 Hz, 1H),9.79 (s, 1H).

### Example 49—Synthesis of N-(6-Methoxy-1-Methyl-1H-Indazol-7-yl)-6-(4-(Trifluoromethyl)-2H-1,2,3-Triazol-2-yl)Pyridine-3-Sulfonamide (I-28)

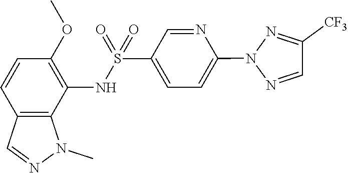 [SMILES: COc1ccc2cnn(C)c2c1NS(=O)(=O)c1ccc(-n2ncc(C(F)(F)F)n2)nc1]

The title compound was obtained using general procedure B as a white solid (3.4 mg, 5%). (ES, m/z): [M+H]+454; 1H NMR (400 MHz, DMSO-d₆) δ 3.17 (s, 3H), 4.29 (s, 3H), 6.82 (d, J=8.9 Hz, 1H), 7.65 (d, J=8.7 Hz, 1H), 7.99 (s, 1H), 8.24-8.48 (m, 2H), 8.81 (dd, J=0.9, 2.2 Hz, 1H), 8.93 (s, 1H), 10.23 (s, 1H).

### Example 50—Synthesis of N-(6-Methoxy-1-Methyl-1H-Indazol-7-yl)-6-(4-(Trifluoromethyl)-1H-1,2,3-Triazol-1-yl)Pyridine-3-Sulfonamide (I-29)

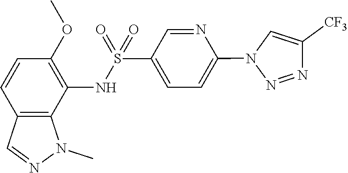 [SMILES: COc1ccc2cnn(C)c2c1NS(=O)(=O)c1ccc(-n2cc(C(F)(F)F)nn2)nc1]

The title compound was obtained using general procedure B as a white solid (3.3 mg, 5.11%). (ES, m/z): [M+H]+ 421; 1H NMR (400 MHz, DMSO-d₆) δ 3.18 (s, 3H), 4.29 (s, 3H), 6.82 (d, J=8.9 Hz, 1H), 7.66 (d, J=8.8 Hz, 1H), 7.99 (s, 1H), 8.39 (d, J=1.6 Hz, 2H), 8.79 (t, J=1.6, 1.6 Hz, 1H), 9.80 (d, J=1.1 Hz, 1H), 10.25 (s, 1H).

### Example 51—Synthesis of N-(6-Methoxy-1-Methylindazol-7-yl)-N-Methyl-6-[3-(Trifluoromethyl) Pyrazol-1-yl] Pyridine-3-Sulfonamide (I-30)

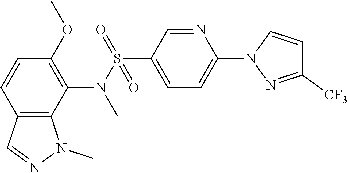 [SMILES: COc1ccc2cnn(C)c2c1N(C)S(=O)(=O)c1ccc(-n2ccc(C(F)(F)F)n2)nc1]

The title compound was obtained using general procedure D as a grey solid (5.2 mg, 6%). (ES, m/z): [M+1]=467; 1H NMR (400 MHz, DMSO-d₆): δ 3.26 (s, 3H), 3.33 (d, J=2.8 Hz, 3H), 4.23 (s, 3H), 6.91 (d, J=8.8 Hz, 1H), 7.17 (d, J=2.4 Hz, 1H), 7.76 (d, J=8.8 Hz, 1H), 8.02 (s, 1H), 8.19 (d, J=8.4 Hz, 1H), 8.32 (dd, J=8.8 Hz, 2.4 Hz, 1H), 8.79 (d, J=2.0 Hz, 1H), 8.95 (d, J=1.6 Hz, 1H).

### Example 52—Synthesis of N-(1,6-Dimethyl-1H-Indazol-7-yl)-6-(4-(Trifluoromethyl)-1H-Pyrazol-1-yl)Pyridine-3-Sulfonamide (I-31)

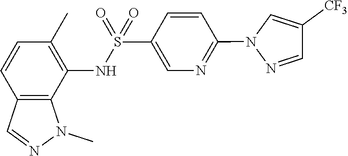 [SMILES: Cc1ccc2cnn(C)c2c1NS(=O)(=O)c1ccc(-n2cc(C(F)(F)F)cn2)nc1]

The title compound was obtained using general procedure B as a light-pink solid (16.5 mg, 9.%). (ES, m/z): [M+H]⁺ 437; ¹H NMR (400 MHz, DMSO-d₆) δ 1.69 (s, 3H), 4.26 (s, 3H), 6.88 (d, J=8.0 Hz, 1H), 7.58 (t, J=6.0 Hz, 1H), 8.01 (s, 1H), 8.18 (d, J=7.2 Hz, 1H), 8.26 (dd, J=8.8 Hz, 2.4 Hz, 1H), 8.42 (s, 1H), 8.65 (d, J=2.4 Hz, 1H), 9.33 (s, 1H), 10.37 (s, 1H).

### Example 53—Synthesis of 6-(4-Isopropyl-1H-Pyrazol-1-yl)-N-(6-Methoxy-1-Methyl-1H-Indazol-7-yl)Pyridine-3-Sulfonamide (I-32)

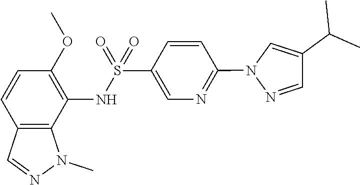 [SMILES: COc1ccc2cnn(C)c2c1NS(=O)(=O)c1ccc(-n2cc(C(C)C)cn2)nc1]

The title compound was obtained using general procedure B as an off-white solid (8 mg, 7%). (ES, m/z): [M+H]⁺ 427; ¹H NMR (400 MHz, DMSO-d₆) δ 1.24 (d, J=6.9 Hz, 6H), 2.90 (p, J=6.9 Hz, 1H), 3.19 (s, 3H), 4.27 (s, 3H), 6.83 (d, J=8.9 Hz, 1H), 7.67 (d, J=8.8 Hz, 1H), 7.85 (s, 1H), 7.99 (s, 1H), 8.05 (d, J=8.7 Hz, 1H), 8.17 (dd, J=2.4, 8.7 Hz, 1H), 8.46 (s, 1H), 8.57 (d, J=2.3 Hz, 1H), 10.00 (s, 1H).

### Example 54—Synthesis of N-(6-Ethyl-1-Methyl-1H-Indazol-7-yl)-6-(3-Methyl-1H-Pyrazol-1-yl)Pyridine-3-Sulfonamide (I-33)

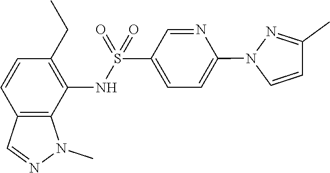 [SMILES: CCc1ccc2cnn(C)c2c1NS(=O)(=O)c1ccc(-n2ccc(C)n2)nc1]

The title compound was obtained using general procedure B as an off-white solid (22.5 mg, 10%). (ES, m/z): [M+H]⁺ 397; ¹H NMR (400 MHz, DMSO-d₆) δ 0.80 (t, J=7.6 Hz, 3H), 2.00-2.10 (m, 2H), 2.32 (s, 3H), 4.25 (s, 3H), 6.48 (d, J=2.4 Hz, 1H), 6.96 (d, J=8.4 Hz, 1H), 7.65 (d, J=8.4 Hz, 1H), 8.01 (s, 1H), 8.03 (d, J=2.4 Hz, 1H), 8.10-8.17 (m, 1H), 8.56 (dd, J=8.4, 2.4 Hz, 2H), 10.28 (s, 1H).

### Example 55—Synthesis of N-(1,6-dimethyl-1H-indazol-7-yl)-6-(3-methyl-1H-pyrazol-1-yl)pyridine-3-sulfonamide (I-34)

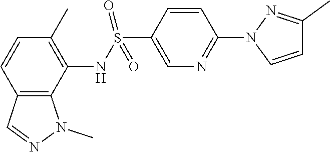 [SMILES: Cc1ccn(-c2ccc(S(=O)(=O)Nc3c(C)ccc4cnn(C)c34)cn2)n1]

The title compound was obtained using general procedure D as a light-pink solid (8.4 mg, 7%). (ES, m/z): [M+H]⁺383; ¹H NMR (400 MHz, DMSO-d₆) δ 1.70 (s, 3H), 2.31 (s, 3H), 4.25 (s, 3H), 6.46 (d, J=2.4 Hz, 1H), 6.84 (d, J=8.0 Hz, 1H), 7.48 (d, J=8.0 Hz, 1H), 7.95-8.10 (m, 3H), 8.56-8.52 (m, 2H), 10.37 (s, 1H).

### Example 56—Synthesis of 6-(4-Methoxy-1H-Pyrazol-1-yl)-N-(1-Methyl-1H-Indazol-7-yl)Pyridine-3-Sulfonamide (I-35)

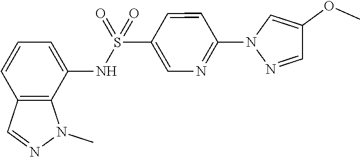 [SMILES: COc1cnn(-c2ccc(S(=O)(=O)Nc3cccc4cnn(C)c34)cn2)c1]

The title compound was obtained using general procedure D as a brown solid (33.5 mg, 14%). (ES, m/z): [M−H]⁺ 383; ¹H NMR (400 MHz, DMSO-d₆) δ 3.81 (s, 3H), 4.29 (s, 3H), 6.53 (d, J=7.3 Hz, 1H), 6.94 (t, J=7.7 Hz, 1H), 7.68 (d, J=8.1 Hz, 1H), 7.79 (d, J=0.9 Hz, 1H), 8.06 (m, 2H), 8.17 (dd, J=8.8, 2.4 Hz, 1H), 8.37 (d, J=0.9 Hz, 1H), 8.54 (m, 1H), 10.25 (s, 1H).

### Example 57—Synthesis of 6-(3-Isopropyl-1H-Pyrazol-1-yl)-N-(1-Methyl-1H-Indazol-7-yl)Pyridine-3-Sulfonamide (I-36)

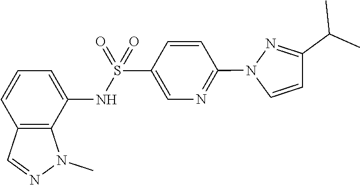 [SMILES: CC(C)c1ccn(-c2ccc(S(=O)(=O)Nc3cccc4cnn(C)c34)cn2)n1]

The title compound was obtained using general procedure D as a dark yellow solid (62.3 mg, 25%). (ES, m/z): [M+H]⁺ 397; ¹H NMR (400 MHz, DMSO-d₆) δ 1.28 (d, J=6.9 Hz, 6H), 3.03 (q, J=6.9 Hz, 1H), 4.29 (s, 3H), 6.54 (m, 2H), 6.94 (t, J=7.7 Hz, 1H), 7.69 (dd, J=8.1, 0.9 Hz, 1H), 8.05 (d, J=8.7 Hz, 1H), 8.10 (s, 1H), 8.18 (dd, J=8.8, 2.4 Hz, 1H), 8.58 (t, J=2.9 Hz, 2H), 10.26 (s, 1H).

### Example 58—Synthesis of 6-(3-Methyl-1H-1,2,4-Triazol-1-yl)-N-(1-Methyl-1H-Indazol-7-yl)Pyridine-3-Sulfonamide (I-37)

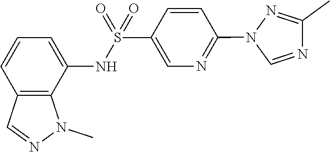 [SMILES: Cc1ncn(-c2ccc(S(=O)(=O)Nc3cccc4cnn(C)c34)cn2)n1]

The title compound was obtained using general procedure B as an off-white solid (7.6 mg, 3%). (ES, m/z): [M+H]⁺ 370; ¹H NMR (400 MHz, DMSO-d₆) δ 2.42 (s, 3H), 4.30 (s, 3H), 6.53 (d, J=7.3 Hz, 1H), 6.94 (t, J=7.7 Hz, 1H), 7.71 (d, J=8.0 Hz, 1H), 8.02 (d, J=8.7 Hz, 1H), 8.11 (s, 1H), 8.28 (dd, J=2.4, 8.6 Hz, 1H), 8.67 (d, J=2.4 Hz, 1H), 9.36 (s, 1H), 10.37 (s, 1H).

### Example 59—Synthesis of 6-(4-Fluoro-1H-Pyrazol-1-yl)-N-(1-Methyl-1H-Indazol-7-yl) Pyridine-3-Sulfonamide (I-38)

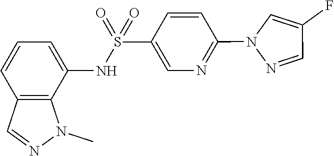 [SMILES: Cn1ncc2cccc(NS(=O)(=O)c3ccc(-n4cc(F)cn4)nc3)c21]

The title compound was obtained using general procedure B to provide the title compound as a white solid (48 mg, 21%). (ES, m/z): [M+1]⁺373; ¹H NMR (400 MHz, DMSO-d₆) δ 4.29 (s, 3H), 6.53 (dd, J=1.0, 7.3 Hz, 1H), 6.94 (t, J=7.7, 7.7 Hz, 1H), 7.70 (dd, J=1.0, 8.1 Hz, 1H), 7.95-8.48 (m, 4H), 8.48-9.01 (m, 2H), 10.32 (s, 1H).

### Example 60—Synthesis of 6-(4-Cyano-1H-Pyrazol-1-yl)-N-(1-Methyl-1H-Indazol-7-yl)Pyridine-3-Sulfonamide (I-39)

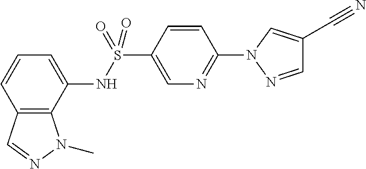 [SMILES: Cn1ncc2cccc(NS(=O)(=O)c3ccc(-n4cc(C#N)cn4)nc3)c21]

The title compound was obtained using general procedure C as a white solid (48.8 mg, 21%). (ES, m/z): [M+H]⁺ 380; ¹H NMR (400 MHz, DMSO-d₆) δ 4.30 (s, 3H), 6.53 (d, J=7.3 Hz, 1H), 6.93 (t, J=7.7, 7.7 Hz, 1H), 7.70 (d, J=8.0 Hz, 1H), 8.10 (s, 1H), 8.20 (d, J=8.7 Hz, 1H), 8.31 (dd, J=2.4, 8.7 Hz, 1H), 8.52 (s, 1H), 8.68 (d, J=2.3 Hz, 1H), 9.54 (s, 1H), 10.39 (s, 1H).

### Example 61—Synthesis of 6-(3,5-Dimethyl-1H-Pyrazol-1-yl)-N-(1-Methyl-1H-Indazol-7-yl)Pyridine-3-Sulfonamide (I-40)

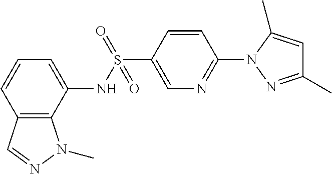 [SMILES: Cc1cc(C)n(-c2ccc(S(=O)(=O)Nc3cccc4cnn(C)c34)cn2)n1]

The title compound was obtained using general procedure B as a white solid (23.4 mg, 10%). (ES, m/z): [M+H]⁺ 383; ¹H NMR (400 MHz, DMSO-d₆) δ 2.23 (s, 3H), 2.63 (s, 3H), 4.28 (s, 3H), 6.22 (s, 1H), 6.53 (d, J=7.3 Hz, 1H), 6.95 (t, J=7.7, 7.7 Hz, 1H), 7.69 (d, J=8.0 Hz, 1H), 8.04 (d, J=8.8 Hz, 1H), 8.09 (s, 1H), 8.14 (dd, J=2.5, 8.8 Hz, 1H), 8.57 (d, J=2.4 Hz, 1H), 10.25 (s, 1H).

### Example 62—Synthesis of N-(1-Methylindazol-7-yl)-6-(4-Nitropyrazol-1-yl)Pyridine-3-Sulfonamide (I-41)

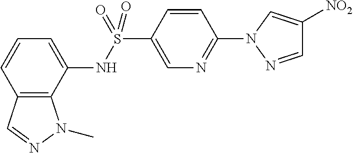 [SMILES: Cn1ncc2cccc(NS(=O)(=O)c3ccc(-n4cc([N+](=O)[O-])cn4)nc3)c21]

The title compound was obtained using general procedure B as a white solid (39 mg, 16%). (ES, m/z): [M+H]⁺ 400; ¹H NMR (400 MHz, DMSO-d₆) δ 4.29 (s, 3H), 6.54 (dd, J=7.3, 1.0 Hz, 1H), 6.93 (t, J=7.7 Hz, 1H), 7.69 (d, J=7.9 Hz, 1H), 8.10 (s, 1H), 8.24 (d, J=8.6 Hz, 1H), 8.33 (dd, J=8.7, 2.3 Hz, 1H), 8.73 (m, 2H), 9.57 (s, 1H), 10.42 (s, 1H).

### Example 63—Synthesis of 6-(4-Chloro-3,5-Dimethyl-1H-Pyrazol-1-yl)-N-(1-Methyl-1H-Indazol-7-yl) Pyridine-3-Sulfonamide (I-42)

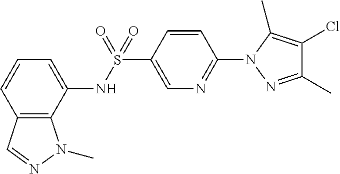 [SMILES: Cc1nn(-c2ccc(S(=O)(=O)Nc3cccc4cnn(C)c34)cn2)c(C)c1Cl]

The title compound was obtained using general procedure B as a white solid (14.6 mg, 11%). (ES, m/z): [M+1]⁺417; ¹H NMR (400 MHz, DMSO-d₆) δ 2.27 (s, 3H), 2.64 (s, 3H), 4.28 (s, 3H), 6.53 (dd, J=1.0, 7.3 Hz, 1H), 6.94 (t, J=7.7 Hz, 1H), 7.66-7.73 (m, 1H), 7.91-8.25 (m, 3H), 8.58-8.64 (m, 1H), 10.30 (s, 1H).

### Example 64—Synthesis of 6-[5-Methyl-3-(Trifluoromethyl)Pyrazol-1-Yl]-N-(1-Methylindazol-7-yl)Pyridine-3-Sulfonamide (I-43)

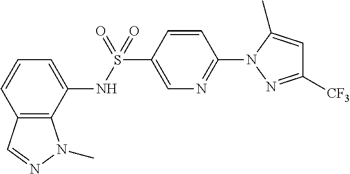 [SMILES: Cc1cc(C(F)(F)F)nn1-c1ccc(S(=O)(=O)Nc2cccc3cnn(C)c23)cn1]

The title compound was obtained using general procedure B as a white solid (41 mg, 15%). (ES, m/z): [M+H]⁺ 437; ¹H NMR (400 MHz, DMSO-d₆) δ 2.70 (m, 3H), 4.31 (s, 3H), 6.56 (dd, J=7.4, 1.0 Hz, 1H), 6.92 (m, 2H), 7.68 (dd, J=8.1, 0.9 Hz, 1H), 8.11 (m, 2H), 8.29 (dd, J=8.7, 2.4 Hz, 1H), 8.70 (d, J=2.3 Hz, 1H), 10.38 (s, 1H).

### Example 65—Synthesis of 6-[4-Methyl-3-(Trifluoromethyl)Pyrazol-1-Yl]-N-(1-Methylindazol-7-yl)Pyridine-3-Sulfonamide (I-44)

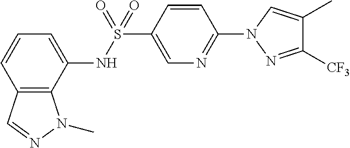 [SMILES: Cc1cn(-c2ccc(S(=O)(=O)Nc3cccc4cnn(C)c34)cn2)nc1C(F)(F)F]

The title compound was obtained using general procedure B as a white solid (35.6 mg, 26%). (ES, m/z): [M+H]⁺ 437; ¹H NMR (400 MHz, DMSO-d₆) δ 2.22 (s, 3H), 4.31 (s, 3H), 6.56 (d, J=7.3 Hz, 1H), 6.89 (t, J=7.7 Hz, 1H), 7.56 (d, J=8.0 Hz, 1H), 8.04 (s, 1H), 8.10 (d, J=8.7 Hz, 1H), 8.27 (dd, J=8.7, 2.3 Hz, 1H), 8.66 (d, J=2.2 Hz, 1H), 8.75 (s, 1H), 10.22 (s, 1H).

### Example 66—Synthesis of Methyl 2-Chloro-4-(5-(N-(6-Ethyl-1-Methyl-1H-Indazol-7-yl)Sulfamoyl)Pyridin-2-yl)Benzoate (I-45) and 2-Chloro-4-(5-(N-(6-Ethyl-1-Methyl-1H-Indazol-7-yl)Sulfamoyl) Pyridin-2-yl)Benzoic Acid

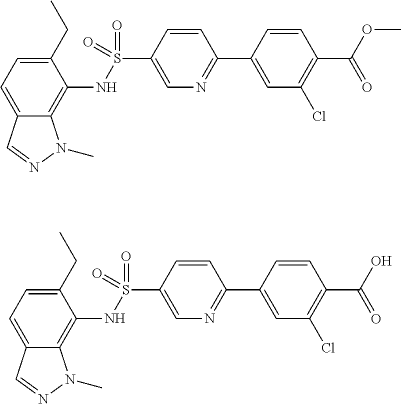 [SMILES: CCc1ccc2cnn(C)c2c1NS(=O)(=O)c1ccc(-c2ccc(C(=O)OC)c(Cl)c2)nc1 ; CCc1ccc2cnn(C)c2c1NS(=O)(=O)c1ccc(-c2ccc(C(=O)O)c(Cl)c2)nc1]

The title compound methyl 2-chloro-4-(5-(N-(6-ethyl-1-methyl-1H-indazol-7-yl)sulfamoyl) pyridin-2-yl)benzoate was obtained using general procedure E as an off white solid (16 mg, 7%). Further to this 2-chloro-4-(5-(N-(6-ethyl-1-methyl-1H-indazol-7-yl)sulfamoyl)pyridin-2-yl)benzoic acid was isolated by acidification of the aqueous layer using aqueous HCl (2 M) to pH 3 and extraction with EtOAc. The organic extract was dried over magnesium sulfate and the solvent removed under reduced pressure to afford 2-chloro-4-(5-(N-(6-ethyl-1-methyl-1H-indazol-7-yl)sulfamoyl)pyridin-2-yl)benzoic acid (120 mg, 0.25 mmol, 60%), which was used immediately without any further purification. (ES, m/z): [M+H]+ 485.2; ¹H NMR (400 MHz, DMSO-d₆) δ 0.75 (t, J=7.5 Hz, 3H), 2.03 (br s, 2H), 3.90 (s, 3H), 4.25 (s, 3H), 6.95 (d, J=8.3 Hz, 1H), 7.64 (d, J=8.3 Hz, 1H), 8.01-7.98 (m, 2H), 8.12 (dd, J=2.4, 8.4 Hz, 1H), 8.25 (dd, J=1.8, 8.3 Hz, 1H), 8.38-8.34 (m, 2H), 8.86 (d, J=2.1 Hz, 1H), 10.39 (br s, 1H). The aqueous phase pH was adjusted to 3 using 2 M aqueous HCl solution and extracted with EtOAc. The organic extract was dried (MgSO4) and the solvent removed under reduced pressure to afford 2-chloro-4-(5-(N-(6-ethyl-1-methyl-1H-indazol-7-yl)sulfamoyl)pyridin-2-yl)benzoic acid which was carried forward to the next step without further purification (120 mg, 0.25 mmol, 60%). (ES, m/z): [M+H]⁺ 471.2.

### Example 67—Synthesis of N-(6-Ethyl-1-Methyl-1H-Indazol-7-yl)-2′-(Trifluoromethyl)-[2,4′-Bipyridine]-5-Sulfonamide (I-46)

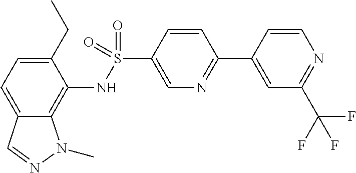 [SMILES: CCc1ccc2cnn(C)c2c1NS(=O)(=O)c1ccc(-c2ccnc(C(F)(F)F)c2)nc1]

The title compound was obtained using general procedure E. (ES, m/z): [M+H]+ 462.5; ¹H NMR (400 MHz, DMSO-d₆) δ 0.75 (t, J=7.5, 3H), 2.02 (s, 2H), 4.25 (s, 3H), 6.95 (d, J=8.3 Hz, 1H), 7.64 (d, J=8.3 Hz, 1H), 8.01 (s, 1H), 8.20 (dd, J=2.4, 8.4 Hz, 1H), 8.47 (dd, J=1.4, 5.0 Hz, 1H), 8.52 (d, J=8.4 Hz, 1H), 8.59 (s, 1H), 8.93 (d, J=2.0 Hz, 1H), 8.97 (d, J=5.1 Hz, 1H), 10.46 (s, 1H).

### Example 68—Synthesis of N-(6-Ethyl-1-Methyl-1H-Indazol-7-yl)-[2,4′-Bipyridine]-5-Sulfonamide (I-47)

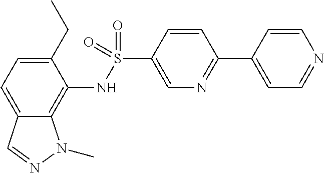 [SMILES: CCc1ccc2cnn(C)c2c1NS(=O)(=O)c1ccc(-c2ccncc2)nc1]

The title compound was obtained using general procedure E. (ES, m/z): [M+H]⁺ 394.2; ¹H NMR (400 MHz, DMSO-d₆) δ 0.75 (t, J=7.5 Hz, 3H), 2.03 (m, 2H), 4.25 (s, 3H), 6.95 (d, J=8.3 Hz, 1H), 7.63 (d, J=8.2 Hz, 1H), 8.00 (s, 1H), 8.17-8.10 (m, 3H), 8.35 (d, J=8.4 Hz, 1H), 8.78-8.76 (m, 2H), 8.88 (d, J=2.0 Hz, 1H), 10.42 (br s, 1H).

### Example 69—Synthesis of 6-[4-(Difluoromethyl)Pyrazol-1-Yl]-N-(1-Methylindazol-7-yl)Pyridine-3-Sulfonamide (I-50)

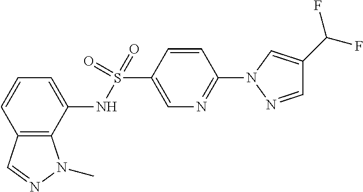 [SMILES: Cn1ncc2cccc(NS(=O)(=O)c3ccc(-n4cc(C(F)F)cn4)nc3)c21]

The title compound was obtained using general procedure B as a white solid. (35 mg, 14%). (ES, m/z): [M+H]⁺ 405; ¹H NMR (400 MHz, DMSO-d₆) δ 4.29 (s, 3H), 6.52 (dd, J=7.3, 1.0 Hz, 1H), 6.94 (t, J=7.7 Hz, 1H), 7.16 (t, J=55.5 Hz, 1H), 7.71 (dd, J=8.1, 1.0 Hz, 1H), 8.11 (s, 1H), 8.19 (m, 2H), 8.27 (dd, J=8.7, 2.4 Hz, 1H), 8.65 (d, J=2.3 Hz, 1H), 9.03 (d, J=2.0 Hz, 1H), 10.34 (s, 1H).

### Example 70—Synthesis of 6-[3-(Difluoromethyl)Pyrazol-1-Yl]-N-(1-Methylindazol-7-yl)Pyridine-3-Sulfonamide (I-51)=

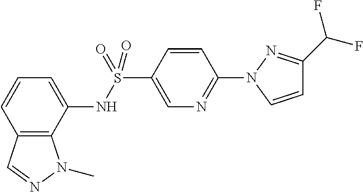 [SMILES: Cn1ncc2cccc(NS(=O)(=O)c3ccc(-n4ccc(C(F)F)n4)nc3)c21]

The title compound was obtained using general procedure B as a white solid (3 mg, 1%). (ES, m/z): [M+H]⁺ 405; ¹H NMR (400 MHz, methanol-d₄) δ 4.37 (s, 3H), 6.81 (m, 4H), 7.32 (dt, J=7.8, 1.1 Hz, 1H), 7.86 (s, 1H), 8.02 (d, J=8.6 Hz, 1H), 8.22 (dd, J=8.6, 2.3 Hz, 1H), 8.70 (m, 2H).

### Example 71—Synthesis of N-(1-Methyl-1H-Indazol-7-yl)-6-(4-Methyl-2H-1,2,3-Triazol-2-yl)Pyridine-3-Sulfonamide (I-52)

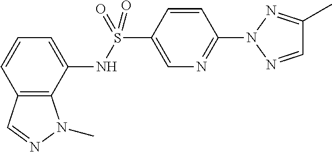 [SMILES: Cc1cnn(-c2ccc(S(=O)(=O)Nc3cccc4cnn(C)c34)cn2)n1]

The title compound was obtained using general procedure B as an off-white solid (18 mg, 15%). (ES, m/z): [M+H]⁺ 370; ¹H NMR (400 MHz, DMSO-d₆) δ 2.41 (s, 3H), 4.30 (s, 3H), 6.54 (d, J=7.3 Hz, 1H), 6.95 (t, J=7.7 Hz, 1H), 7.72 (d, J=8.0 Hz, 1H), 8.06-8.14 (m, 2H), 8.19 (d, J=8.7 Hz, 1H), 8.27 (dd, J=2.4, 8.7 Hz, 1H), 8.72 (d, J=2.3 Hz, 1H), 10.38 (s, 1H).

### Example 72—Synthesis of 6-(4-Methyl-1H-1,2,3-Triazol-1-yl)-N-(1-Methyl-1H-Indazol-7-yl)Pyridine-3-Sulfonamide (I-53)

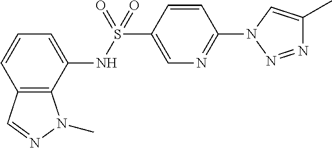 [SMILES: Cc1cn(-c2ccc(S(=O)(=O)Nc3cccc4cnn(C)c34)cn2)nn1]

The title compound was obtained using general procedure B as a white solid (6 mg, 5%). (ES, m/z): [M+H]⁺ 370; ¹H NMR (400 MHz, DMSO-d₆) δ 2.37 (s, 3H), 4.31 (s, 3H), 6.57 (d, J=7.3 Hz, 1H), 6.91 (t, J=7.7 Hz, 1H), 7.61 (d, J=8.1 Hz, 1H), 8.06 (s, 1H), 8.27-8.37 (m, 2H), 8.67-8.75 (m, 2H), 10.43 (s, 1H).

### Example 73—Synthesis of 6-(4,5-Dimethylpyrazol-1-yl)-N-(1-Methylindazol-7-yl)Pyridine-3-Sulfonamide (I-57)

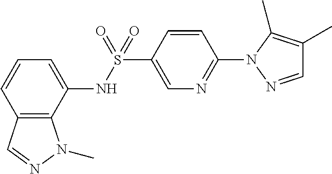 [SMILES: Cc1cnn(-c2ccc(S(=O)(=O)Nc3cccc4cnn(C)c34)cn2)c1C]

The title compound was obtained using general procedure B as an off-white solid (3 mg, 1%). (ES, m/z): [M+H]⁺ 383; ¹H NMR (400 MHz, DMSO-d₆) δ 2.02 (s, 3H), 2.57 (s, 3H), 4.29 (s, 3H), 6.56 (d, J=7.3 Hz, 1H), 6.92 (t, J=7.7 Hz, 1H), 7.69-7.60 (m, 2H), 8.20-8.02 (m, 3H), 8.62-8.57 (m, 1H), 10.26 (s, 1H).

### Example 74—Synthesis of 6-(3,4-Dimethylpyrazol-1-yl)-N-(1-Methylindazol-7-yl)Pyridine-3-Sulfonamide (I-58)

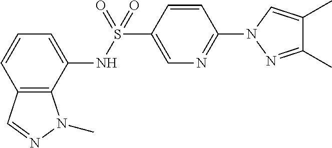 [SMILES: Cc1cn(-c2ccc(S(=O)(=O)Nc3cccc4cnn(C)c34)cn2)nc1C]

The title compound was obtained using general procedure B as an off-white solid (16 mg, 7%). (ES, m/z): [M+H]⁺ 383; ¹H NMR (400 MHz, DMSO-d₆) δ 2.04 (s, 3H), 2.23 (s, 3H), 4.29 (s, 3H), 6.53 (d, J=7.3 Hz, 1H), 6.93 (t, J=7.7 Hz, 1H), 7.68 (d, J=8.0 Hz, 1H), 7.98 (d, J=8.7 Hz, 1H), 8.09 (s, 1H), 8.14 (dd, J=8.8, 2.4 Hz, 1H), 8.37 (s, 1H), 8.54 (d, J=2.4 Hz, 1H), 10.23 (s, 1H).

### Example 75—Synthesis of N-(1-Methyl-1H-Indazol-7-yl)-6-(3,4,5-Trimethyl-1H-Pyrazol-1-yl)Pyridine-3-Sulfonamide (I-59)

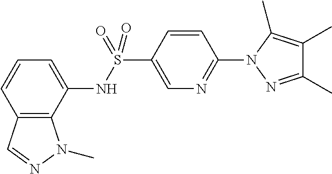 [SMILES: Cc1nn(-c2ccc(S(=O)(=O)Nc3cccc4cnn(C)c34)cn2)c(C)c1C]

The title compound was obtained using general procedure B as a pink solid (8 mg, 6%). (ES, m/z): [M+H]⁺ 397; ¹H NMR (400 MHz, DMSO-d₆) δ 1.94 (s, 3H), 2.19 (s, 3H), 2.55 (s, 3H), 4.29 (s, 3H), 6.54 (d, J=7.3 Hz, 1H), 6.92 (t, J=7.7, 7.7 Hz, 1H), 7.63 (d, J=8.0 Hz, 1H), 7.99 (d, J=8.8 Hz, 1H), 8.07 (s, 1H), 8.12 (dd, J=2.4, 8.8 Hz, 1H), 8.55 (d, J=2.5 Hz, 1H), 10.23 (s, 1H).

### Example 76—Synthesis of 6-(5-Cyclopropylpyrazol-1-yl)-N-(1-Methylindazol-7-yl)Pyridine-3-Sulfonamide (I-62)

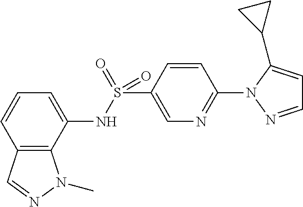 [SMILES: Cn1ncc2cccc(NS(=O)(=O)c3ccc(-n4nccc4C4CC4)nc3)c21]

The title compound was obtained using general procedure B as a white solid (40 mg, 16%). (ES, m/z): [M+H]⁺ 395; ¹H NMR (400 MHz, DMSO-d₆) δ 0.77-0.89 (m, 2H), 0.93-1.04 (m, 2H), 1.98-2.11 (m, 1H), 4.31 (s, 3H), 6.39 (d, J=2.7 Hz, 1H), 6.54 (d, J=7.1 Hz, 1H), 6.93 (t, J=7.7 Hz, 1H), 7.67 (d, J=8.0 Hz, 1H), 8.01 (d, J=8.7 Hz, 1H), 8.09 (s, 1H), 8.18 (dd, J=8.8, 2.4 Hz, 1H), 8.56 (dd, J=14.1, 2.6 Hz, 2H), 10.24 (s, 1H).

### Exampel 77—Synthesis of N-(1-Methyl-1H-Benzo[D][1,2,3]Triazol-7-yl)-6-(4-(Trifluoromethyl)-1H-Pyrazol-1-yl)Pyridine-3-Sulfonamide (I-64)

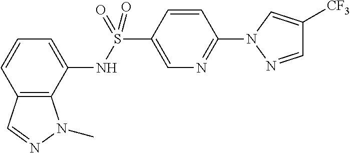 [SMILES: Cn1ncc2cccc(NS(=O)(=O)c3ccc(-n4cc(C(F)(F)F)cn4)nc3)c21]

The title compound was obtained using general procedure B as a white solid (16 mg, 10%). (ES, m/z): [M+H]⁺ 424; ¹H NMR (400 MHz, DMSO-d₆) δ 4.51 (s, 3H), 6.71 (d, J=7.2 Hz, 1H), 7.20 (t, J=8.0 Hz, 1H), 7.92 (d, J=8.0 Hz, 1H), 7.18 (d, J=8.8 Hz, 1H), 8.29 (dd, J=3.0, 8.8 Hz, 1H), 8.42 (s, 1H), 8.66 (s, 1H), 9.32 (s, 1H), 10.61 (s, 1H).

### Example 78—Synthesis of 5-Methyl-N-(1-Methyl-1H-Indazol-7-yl)-6-(4-(Trifluoromethyl)-1H-Pyrazol-1-yl)Pyridine-3-Sulfonamide (I-65)

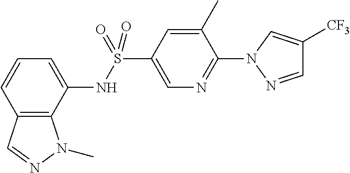 [SMILES: Cc1cc(S(=O)(=O)Nc2cccc3cnn(C)c23)cnc1-n1cc(C(F)(F)F)cn1]

The title compound was obtained using general procedure A as an off-white solid (20 mg, 15%). (ES, m/z): [M+H]⁺ 437; ¹H NMR (400 MHz, DMSO-d₆) 2.54 (s, 3H), 4.31 (s, 3H), 6.58 (d, J=7.3 Hz, 1H), 6.91 (t, J=7.6 Hz, 1H), 7.60 (d, J=7.9 Hz, 1H), 8.05 (s, 1H), 8.20 (s, 1H), 8.34 (s, 1H), 8.58 (s, 1H), 9.09 (s, 1H), 10.40 (s, 1H).

### Example 79—Synthesis of 2-Methyl-N-(1-Methylindazol-7-yl)-6-[4-(Trifluoromethyl)Pyrazol-1-Yl]Pyridine-3-Sulfonamide (I-66)

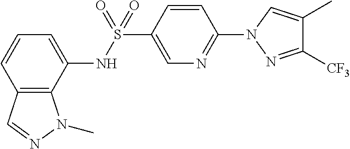 [SMILES: Cc1cn(-c2ccc(S(=O)(=O)Nc3cccc4cnn(C)c34)cn2)nc1C(F)(F)F]

The title compound was obtained using general procedure B as an off-white solid (32 mg, 29%). (ES, m/z): [M+H]⁺ 437; ¹H NMR (400 MHz, DMSO-d₆) δ 2.56 (s, 3H), 4.45 (s, 3H), 6.43-6.52 (m, 2H), 6.86 (t, J=8.0 Hz, 1H), 7.70 (d, J=8.0 Hz, 1H), 7.97 (s, 1H), 7.99-8.12 (m, 2H), 8.72 (s, 1H), 8.97 (m, 1H).

### Example 80—Synthesis of 4-Methyl-N-(1-Methylindazol-7-yl)-6-[4-(Trifluoromethyl)Pyrazol-1-Yl]Pyridine-3-Sulfonamide (I-67

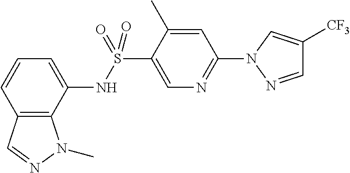 [SMILES: Cc1cc(-n2cc(C(F)(F)F)cn2)ncc1S(=O)(=O)Nc1cccc2cnn(C)c12]

The title compound was obtained using general procedure A as an off-white solid (39 mg, 29%). (ES, m/z): [M+H]⁺437; ¹H NMR (400 MHz, DMSO-d₆) δ 2.56 (d, J=2.4 Hz, 3H), 4.46 (s, 3H), 6.45 (d, J=7.6 Hz, 1H), 6.55 (s, 1H), 6.86 (t, J=8.0 Hz, 1H), 7.71 (d, J=8.0 Hz, 1H), 7.91 (d, J=8.8 Hz, 1H), 7.96 (s, 1H), 8.03 (s, 1H), 8.16 (d, J=8.8 Hz, 1H), 8.95 (s, 1H).

### Example 81—Synthesis of N-(6-(Methoxy-D3)-1-Methyl-1H-Indazol-7-yl)-6-(4-(Trifluoromethyl)-1H-Pyrazol-1-yl)Pyridine-3-Sulfonamide (I-68)

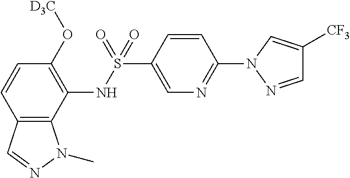 [SMILES: [2H]C([2H])([2H])Oc1ccc2cnn(C)c2c1NS(=O)(=O)c1ccc(-n2cc(C(F)(F)F)cn2)nc1]

The title compound was obtained using general procedure A as a white solid (27 mg, 12%). (ES, m/z): [M+H]⁺ 451; ¹H NMR (400 MHz, DMSO-d₆) δ 4.27 (s, 3H), 6.82 (d, J=8.8 Hz, 1H), 7.69 (d, J=8.8 Hz, 1H), 8.00 (s, 1H), 8.18 (d, J=8.6 Hz, 1H), 8.28 (dd, J=8.7, 2.4 Hz, 1H), 8.42 (s, 1H), 8.68 (d, J=2.3 Hz, 1H), 9.34 (d, J=1.4 Hz, 1H),10.14 (s, 1H).

### Example 82—Synthesis of N-(1-Methyl-6-(Methyl-Ds)-1H-Indazol-7-yl)-6-(4-(Trifluoromethyl)-1H-Pyrazol-1-yl)Pyridine-3-Sulfonamide (I-69)

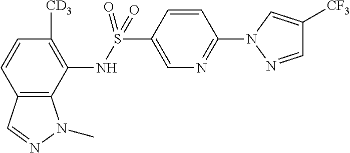 [SMILES: [2H]C([2H])([2H])c1ccc2cnn(C)c2c1NS(=O)(=O)c1ccc(-n2cc(C(F)(F)F)cn2)nc1]

The title compound was obtained using general procedure A as a white solid (41 mg, 14%). ES, m/z): [M+H]⁺ 451; ¹H NMR (400 MHz, DMSO-d₆) δ 4.26 (s, 3H), 6.88 (d, J=8.2 Hz, 1H), 7.60 (d, J=8.1 Hz, 1H), 8.01 (s, 1H), 8.19 (d, J=8.6 Hz, 1H), 8.27 (dd, J=2.4, 8.6 Hz, 1H), 8.42 (s, 1H), 8.66 (d, J=2.3 Hz, 1H), 9.33 (s, 1H), 10.38 (s, 1H).

### Example 83—Synthesis of N-(6-(Ethyl-1,1-D2)-1-Methyl-1H-Indazol-7-yl)-6-(4-(Trifluoromethyl)-1H-Pyrazol-1-yl)Pyridine-3-Sulfonamide (I-70)

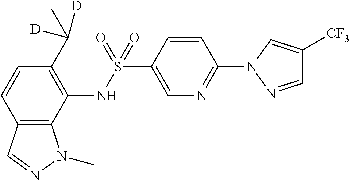 [SMILES: [2H]C([2H])(C)c1ccc2cnn(C)c2c1NS(=O)(=O)c1ccc(-n2cc(C(F)(F)F)cn2)nc1]

The title compound was obtained using general procedure A as a white solid (64 mg, 24%) as a light yellow solid. (ES, m/z): [M+H]⁺ 453; ¹H NMR (400 MHz, DMSO-d₆) δ 0.78 (s, 3H), 4.26 (s, 3H), 6.96 (d, J=8.0 Hz, 1H), 7.66 (t, J=8.4 Hz, 1H), 8.02 (s, 1H), 8.19 (d, J=8.4 Hz, 1H), 8.26 (d, J=2.4 Hz, 1H), 8.42 (s, 1H), 8.64 (d, J=2.0 Hz, 1H), 9.33 (s, 1H), 10.40 (s, 1H).

### Example 84—Synthesis of N-(1,5-Dimethyl-1H-Indazol-7-yl)-6-(4-(Trifluoromethyl)-1H-Pyrazol-1-yl) Pyridine-3-Sulfonamide (I-71)

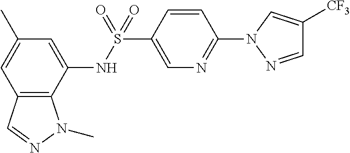 [SMILES: Cc1cc(NS(=O)(=O)c2ccc(-n3cc(C(F)(F)F)cn3)nc2)c2c(cnn2C)c1]

The title compound was obtained using general procedure A as a white solid (54 mg, 17%). (ES, m/z): [M+H]⁺ 437; ¹H NMR (400 MHz, DMSO-d₆) δ 2.17 (s, 3H), 4.24 (s, 3H), 6.36 (d, J=1.2 Hz, 1H), 7.48 (t, J=1.2 Hz, 1H), 7.99 (s, 1H), 8.21 (d, J=8.8 Hz, 1H), 8.29 (dd, J=8.8, 2.4 Hz, 1H), 8.43 (s, 1H), 8.68 (d, J=2.4 Hz, 1H), 9.34 (s, 1H), 10.36 (s, 1H).

### Example 85—Synthesis of N-(5-Methoxy-1-Methylindazol-7-yl)-6-[4-(Trifluoromethyl)Pyrazol-1-Yl]Pyridine-3-Sulfonamide (I-72)

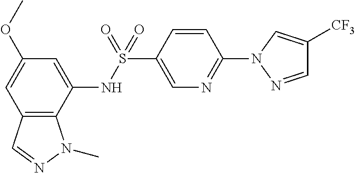 [SMILES: COc1cc(NS(=O)(=O)c2ccc(-n3cc(C(F)(F)F)cn3)nc2)c2c(cnn2C)c1]

The title compound was obtained using general procedure A as an off-white solid (11 mg, 3%). (ES, m/z): [M+H]⁺ 453; ¹H NMR (400 MHz, DMSO-d₆) δ 3.64 (s, 3H), 4.22 (s, 3H), 6.21 (d, J=2.4 Hz, 1H), 7.12 (s, 1H), 7.95 (s, 1H), 8.20 (d, J=8.8 Hz, 1H), 8.33 (dd, J=8.8, 2.4 Hz, 1H), 8.42 (s, 1H), 8.72 (d, J=1.6 Hz, 1H), 9.32 (s, 1H), 10.43 (s, 1H).

### Example 86—Synthesis of N-(5-Fluoro-1-Methylindazol-7-yl)-6-[4-(Trifluoromethyl)Pyrazol-1-Yl]Pyridine-3-Sulfonamide (I-73)

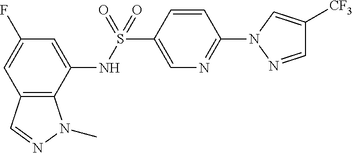 [SMILES: Cn1ncc2cc(F)cc(NS(=O)(=O)c3ccc(-n4cc(C(F)(F)F)cn4)nc3)c21]

The title compound was obtained using general procedure A as an off-white solid (11 mg, 3%). (ES, m/z): [M+H]⁺ 441; ¹H NMR (400 MHz, DMSO-d₆) δ 4.25 (s, 3H), 6.52 (dd, J=9.6, 2.4 Hz, 1H), 7.54 (s, 1H), 8.09 (s, 1H), 8.20 (d, J=8.8 Hz, 1H), 8.32 (dd, J=8.8, 2.4 Hz, 1H), 8.42 (s, 1H), 8.75 (d, J=2.0, 1H), 9.32 (s, 1H), 10.59 (s, 1H).

### Example 87—Synthesis of N-(6-Methoxy-1-(Methyl-D₃)-1H-Indazol-7-yl)-6-(4-(Trifluoromethyl)-1H-Pyrazol-1-yl)Pyridine-3-Sulfonamide (I-75)

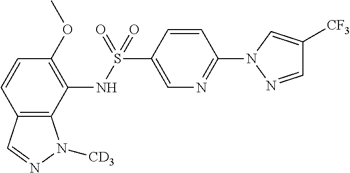 [SMILES: [2H]C([2H])([2H])n1ncc2ccc(OC)c(NS(=O)(=O)c3ccc(-n4cc(C(F)(F)F)cn4)nc3)c21]

The title compound was obtained using general procedure A as a white solid (56 mg, 17%). (ES, m/z): [M+H]⁺ 456; ¹H NMR (400 MHz, DMSO-d₆) δ 3.18 (s, 3H), 6.83 (d, J=8.9 Hz, 1H), 7.69 (d, J=8.8 Hz, 1H), 8.00 (s, 1H), 8.18 (d, J=8.6 Hz, 1H), 8.28 (dd, J=8.6, 2.4 Hz, 1H), 8.41 (s, 1H), 8.68 (d, J=2.4 Hz, 1H), 9.33 (d, J=1.4 Hz, 1H), 10.12 (s, 1H).

### Example 88—Synthesis of 6-(1H-Indol-2-yl)-N-(1-Methyl-1H-Indazol-7-yl)Pyridine-3-Sulfonamide (1-76)

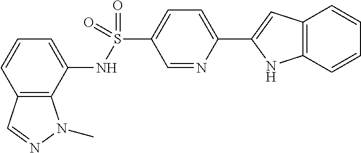 [SMILES: Cn1ncc2cccc(NS(=O)(=O)c3ccc(-c4cc5ccccc5[nH]4)nc3)c21]

The title compound was obtained using general procedure D as a white solid (17 mg, 7%). (ES, m/z): [M+H]⁺ 404.1, ¹H NMR (400 MHz, DMSO-d₆) δ 4.31 (s, 3H), 6.58 (d, J=7.3 Hz, 1H), 6.87 (d, J=3.6 Hz, 1H), 6.94 (t, J=7.6 Hz, 1H), 7.28-7.20 (m, 1H), 7.35-7.30 (m, 1H), 7.67 (d, J=7.5 Hz, 2H), 8.02 (d, J=8.8 Hz, 1H), 8.09 (s, 1H), 8.21-8.11 (m, 2H), 8.57 (d, J=8.3 Hz, 1H), 8.72 (d, J=2.4 Hz, 1H), 10.25 (s, 1H).

### Example 89—Synthesis of N-(1-Methyl-1H-Indazol-7-yl)-6-(1H-Pyrrolo[3,2-C]Pyridin-2-yl)Pyridine-3-Sulfonamide (I-77)

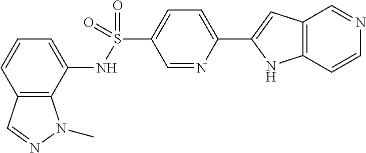 [SMILES: Cn1ncc2cccc(NS(=O)(=O)c3ccc(-c4cc5cnccc5[nH]4)nc3)c21]

The title compound was obtained using general procedure D as an off-white solid (11 mg, 8%). (ES, m/z): [M+H]⁺ 405, ¹H NMR (400 MHz, DMSO-d₆) δ 4.32 (s, 3H), 6.55-6.65 (m, 1H), 6.80-6.95 (m, 1H), 7.40-7.55 (m, 3H), 7.99 (s, 1H), 8.12 (dd, J=8.4, 2.4 Hz, 1H), 8.15-8.25 (m, 2H), 8.79 (s, 1H), 8.91 (s, 1H), 10.41 (s, 1H), 12.29 (s, 1H).

### Example 90—Synthesis of N-(1-Methyl-1H-Indazol-7-yl)-6-(1H-Pyrrol-3-yl)Pyridine-3-Sulfonamide (I-79)

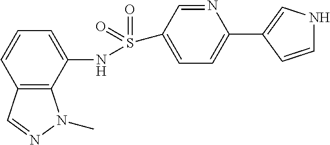 [SMILES: Cn1ncc2cccc(NS(=O)(=O)c3ccc(-c4cc[nH]c4)nc3)c21]

The title compound was obtained using general procedure D as a white solid (22 mg, 19%). (ES, m/z): [M+H]⁺ 354; ¹H NMR (400 MHz, DMSO-d₆) δ 4.28 (s, 3H), 6.53 (d, J=7.2 Hz, 1H), 6.71 (q, J=2.4 Hz, 1H), 6.88 (q, J=2.4 Hz, 1H), 6.93 (t, J=7.6 Hz, 1H), 7.63-7.57 (m, 1H), 7.65 (d, J=8.0 Hz, 1H), 7.78 (d, J=8.5 Hz, 1H), 7.90-7.82 (m, 1H), 8.08 (s, 1H), 8.59 (d, J=2.4 Hz, 1H), 10.12 (s, 1H), 11.32 (s, 1H).

### Example 91—Synthesis of N-(1-Methyl-1H-Indazol-7-yl)-6-(1H-Pyrrol-2-yl)Pyridine-3-Sulfonamide (I-80)

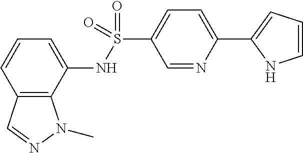 [SMILES: Cn1ncc2cccc(NS(=O)(=O)c3ccc(-c4ccc[nH]4)nc3)c21]

The title compound was obtained using general procedure D as a white solid (25 mg, 22%). (ES, m/z): [M+H]⁺ 354; ¹H NMR (400 MHz, DMSO-d₆) δ 4.29 (s, 3H), 6.20-6.23 (m, 1H), 6.65-6.38 (m, 1H), 6.77-7.14 (m, 3H), 7.67 (d, J=8.1 Hz, 1H), 7.83-7.89 (m, 1H), 7.89-7.96 (m, 1H), 8.09 (s, 1H), 8.39-8.76 (m, 1H), 10.13 (s, 1H), 11.82 (s, 1H).

### Example 92—Synthesis of N-(1-Methyl-1H-Indazol-7-yl)-6-(1H-Pyrazol-5-yl)Pyridine-3-Sulfonamide (I-81)

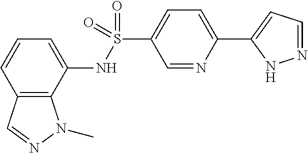 [SMILES: Cn1ncc2cccc(NS(=O)(=O)c3ccc(-c4ccn[nH]4)nc3)c21]

The title compound was obtained using general procedure D as an off-white solid (8 mg, 5%). (ES, m/z): [M+H]⁺ 355; ¹H NMR (400 MHz, DMSO-d₆) δ 4.28 (s, 3H), 6.53 (d, J=7.3 Hz, 1H), 6.96-6.87 (m, 2H), 7.64 (d, J=8.5 Hz, 1H), 7.89 (s, 1H), 8.07 (s, 2H), 8.16 (d, J=8.5 Hz, 1H), 8.74 (d, J=2.4 Hz, 1H), 10.27 (s, 1H), 13.32 (s, 1H).

### Example 93—Synthesis of N-(1-Methyl-1H-Indazol-7-yl)-6-(1-Methyl-1H-Pyrazol-5-yl)Pyridine-3-Sulfonamide (I-82)

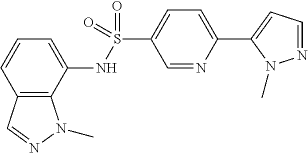 [SMILES: Cn1nccc1-c1ccc(S(=O)(=O)Nc2cccc3cnn(C)c23)cn1]

The title compound was obtained using general procedure D as an off-white solid (48 mg, 42%). (ES, m/z): [M+H]⁺ 369; ¹H NMR (400 MHz, DMSO-d₆) δ 4.19 (s, 3H), 4.29 (s, 3H), 6.52 (d, J=7.4 Hz, 1H), 6.95 (t, J=7.6 Hz, 1H), 7.03 (d, J=2.8 Hz, 1H), 7.57 (s, 1H), 7.75-7.68 (m, 1H), 8.12-8.03 (m, 1H), 8.11 (s, 2H), 8.83 (s, 1H), 10.35 (s, 1H).

### Example 94—Synthesis of N-(1-Methyl-1H-Indazol-7-yl)-6-(3-Methyl-1H-Pyrazol-5-yl)Pyridine-3-Sulfonamide (I-83)

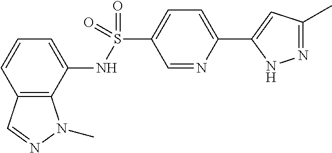 [SMILES: Cc1cc(-c2ccc(S(=O)(=O)Nc3cccc4cnn(C)c34)cn2)[nH]n1]

The title compound was obtained using general procedure D as a pale grey solid (7 mg, 6%). (ES, m/z): [M+H]⁺ 369; ¹H NMR (400 MHz, DMSO-d₆) δ 2.31 (s, 3H), 4.27 (s, 3H), 6.49 (d, J=7.2 Hz, 1H), 6.67 (s, 1H), 6.97-6.90 (m, 1H), 7.72-7.68 (m, 1H), 8.05-8.01 (m, 1H), 8.11 (d, J=3.8 Hz, 2H), 8.70 (d, J=2.8 Hz, 1H), 10.24 (s, 1H), 13.02 (s, 1H).

### Example 95—Synthesis of N-(1-Methyl-1H-Indazol-7-yl)-6-(3-(Trifluoromethyl)-1H-Pyrazol-4-yl)Pyridine-3-Sulfonamide (I-85)

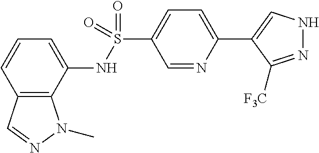 [SMILES: Cn1ncc2cccc(NS(=O)(=O)c3ccc(-c4c[nH]nc4C(F)(F)F)nc3)c21]

The title compound was obtained using general procedure D as an off-white solid (26 mg, 20%). (ES, m/z): [M+H]⁺ 423; ¹H NMR (400 MHz, DMSO-d₆) δ 4.28 (s, 3H) 6.50 (d, J=7.3 Hz, 1H), 6.92 (t, J=7.6 Hz, 1H), 7.66 (d, J=8.0 Hz, 1H), 7.87 (d, J=8.4 Hz, 1H), 8.11-8.04 (m, 2H), 8.69 (s, 1H), 8.77 (dd, J=2.4, 0.8 Hz, 1H), 10.30 (s, 1H), 14.01 (s, 1H).

### Example 96—Synthesis of 6-(1-(Difluoromethyl)-1H-Pyrazol-4-yl)-N-(1-Methyl-1H-Indazol-7-yl)Pyridine-3-Sulfonamide (I-86)

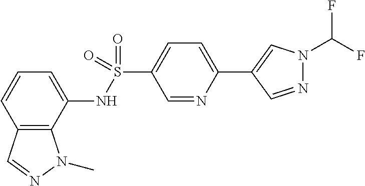 [SMILES: Cn1ncc2cccc(NS(=O)(=O)c3ccc(-c4cnn(C(F)F)c4)nc3)c21]

The title compound was obtained using general procedure D as an off-white solid (54 mg, 43%). (ES, m/z): [M+H]⁺ 405; ¹H NMR (400 MHz, DMSO-d₆) δ 4.28 (s, 3H), 6.50 (d, J=7.2 Hz, 1H), 6.92 (t, J=7.6 Hz, 1H), 7.66 (d, J=8.0 Hz, 1H), 7.91 (t, J=58.8 Hz, 1H), 8.11-8.01 (m, 3H), 8.48 (s, 1H), 8.74 (t, J=1.5 Hz, 1H), 9.01 (s, 1H), 10.27 (s, 1H).

### Example 97—Synthesis of 6-(1,3-Dimethyl-1H-Pyrazol-4-yl)-N-(1-Methyl-1H-Indazol-7-yl)Pyridine-3-Sulfonamide (I-87)

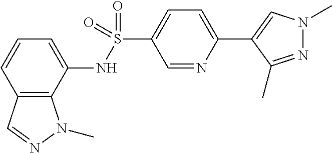 [SMILES: Cc1nn(C)cc1-c1ccc(S(=O)(=O)Nc2cccc3cnn(C)c23)cn1]

The title compound was obtained using general procedure D as an off-white solid (48 mg, 42%). (ES, m/z): [M+H]⁺ 383; ¹H NMR (400 MHz, DMSO-d₆) δ 2.46 (s, 3H), 3.82 (s, 3H), 4.27 (s, 3H), 6.50 (d, J=7.3 Hz, 1H), 6.94 (t, J=7.7 Hz, 1H), 7.70 (d, J=8.0 Hz, 1H), 7.76 (d, J=8.5 Hz, 1H), 7.95 (dd, J=8.5, 2.5 Hz, 1H), 8.10 (s, 1H), 8.37 (s, 1H), 8.68 (d, J=2.4 Hz, 1H), 10.19 (s, 1H).

### Example 98—Synthesis of 6-(1,5-Dimethyl-1H-Pyrazol-4-yl)-N-(1-Methyl-1H-Indazol-7-yl)Pyridine-3-Sulfonamide (I-88)

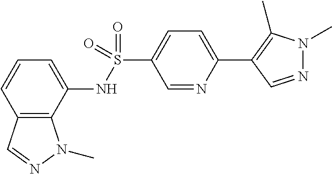 [SMILES: Cc1c(-c2ccc(S(=O)(=O)Nc3cccc4cnn(C)c34)cn2)cnn1C]

The title compound was obtained using general procedure D as a white solid (37 mg, 31%). (ES, m/z): [M+H]⁺ 383; ¹H NMR (400 MHz, DMSO-d₆) δ 2.63 (s, 3H), 3.80 (s, 3H), 4.28 (s, 3H), 6.51 (d, J=7.2 Hz, 1H), 6.94 (t, J=7.6 Hz, 1H), 7.70 (d, J=8.0 Hz, 1H), 7.85 (d, J=8.4 Hz, 1H), 7.96 (dd, J=8.4, 2.4 Hz, 1H), 8.05 (s, 1H), 8.10 (s, 1H), 8.69 (d, J=2.4 Hz, 1H), 10.21 (s, 1H).

### Example 99—Synthesis of N-(1-Methyl-1H-Indazol-7-yl)-6-(1,3,5-Trimethyl-1H-Pyrazol-4-yl)Pyridine-3-Sulfonamide (I-89

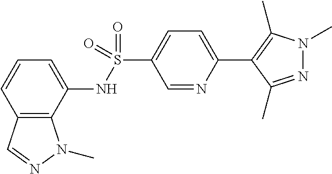 [SMILES: Cc1nn(C)c(C)c1-c1ccc(S(=O)(=O)Nc2cccc3cnn(C)c23)cn1]

The title compound was obtained using general procedure D as a white solid (51 mg, 42%). (ES, m/z): [M+H]⁺ 397; ¹H NMR (400 MHz, DMSO-d₆) δ 2.31 (s, 3H), 2.45 (s, 3H), 3.74 (s, 3H), 4.26 (s, 3H), 6.56-6.49 (m, 1H), 6.95 (t, J=7.7 Hz, 1H), 7.62 (d, J=8.6 Hz, 1H), 7.71 (d, J=8.1 Hz, 1H), 8.02-7.96 (m, 1H), 8.10 (s, 1H), 8.73 (d, J=2.4 Hz, 1H), 10.22 (s, 1H).

### Example 100—Synthesis of 6-(1-Methyl-1H-Imidazol-5-yl)-N-(1-Methyl-1H-Indazol-7-yl)Pyridine-3-Sulfonamide (I-90)

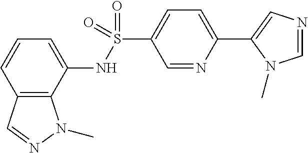 [SMILES: Cn1cncc1-c1ccc(S(=O)(=O)Nc2cccc3cnn(C)c23)cn1]

The title compound was obtained using general procedure D as an off-white solid (59 mg, 51%). (ES, m/z): [M+H]⁺ 369; ¹H NMR (400 MHz, DMSO-d₆) δ 3.99 (s, 3H), 4.29 (s, 3H), 6.53 (dd, J=7.2, 1.0 Hz, 1H), 6.96 (t, J=7.6 Hz, 1H), 7.74 (dd, J=8.0, 1.0 Hz, 1H), 7.82 (s, 1H), 7.97 (s, 1H), 7.99-8.09 (m, 2H), 8.14 (d, J=9.4 Hz, 2H), 8.73 (dd, J=2.4, 1.0 Hz, 1H).

### Example 101—Synthesis of N-(1-Methyl-1H-Indazol-7-yl)-6-(3-Methylisoxazol-5-yl)Pyridine-3-Sulfonamide (I-98)

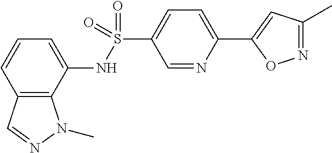 [SMILES: Cc1cc(-c2ccc(S(=O)(=O)Nc3cccc4cnn(C)c34)cn2)on1]

The title compound was obtained using general procedure D as a white solid (12 mg, 10%). (ES, m/z): [M+H]⁺ 370; ¹H NMR (400 MHz, DMSO-d₆) δ 2.35 (s, 3H), 4.28 (s, 3H), 6.51 (d, J=7.3 Hz, 1H), 6.92 (t, J=7.7 Hz, 1H), 7.17 (s, 1H), 7.66 (d, J=8.3 Hz, 1H), 8.08 (s, 1H), 8.14 (d, J=8.3 Hz, 1H), 8.21 (dd, J=8.3, 2.3 Hz, 1H), 8.87 (dd, J=2.3, 0.9 Hz, 1H), 10.42 (s, 1H).

### Example 102—Synthesis of N-(1-Methyl-1H-Indazol-7-yl)-6-(Thiazol-5-yl)Pyridine-3-Sulfonamide (I-101)

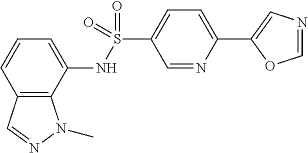 [SMILES: Cn1ncc2cccc(NS(=O)(=O)c3ccc(-c4cnco4)nc3)c21]

The title compound was obtained using general procedure D as a white solid (24 mg, 20%). (ES, m/z): [M+H]⁺ 372; ¹H NMR (400 MHz, DMSO-d₆) δ 4.28 (s, 3H), 6.51 (dd, J=7.3, 1.0 Hz, 1H), 6.94 (dd, J=8.1, 7.3 Hz, 1H), 7.70 (d, J=8.0 Hz, 1H), 8.07-8.14 (m, 2H), 8.27 (dd, J=8.4, 0.9 Hz, 1H), 8.73-8.82 (m, 2H), 9.28 (d, J=0.6 Hz, 1H), 10.34 (s, 1H).

### Example 103—Synthesis of 6-(2,4-Dimethylthiazol-5-yl)-N-(1-Methyl-1H-Indazol-7-yl)Pyridine-3-Sulfonamide (I-103)

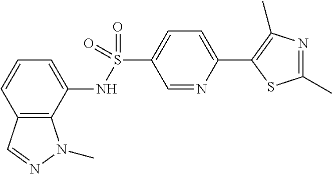 [SMILES: Cc1nc(C)c(-c2ccc(S(=O)(=O)Nc3cccc4cnn(C)c34)cn2)s1]

The title compound was obtained using general procedure D as an off-white solid. (50 mg, 40%). (ES, m/z): [M+H]⁺ 400; ¹H NMR (400 MHz, DMSO-d₆) δ 2.65 (s, 6H), 4.28 (s, 3H), 6.53 (dd, J=0.9, 7.3 Hz, 1H), 6.94 (t, J=7.7, 7.7 Hz, 1H), 7.59-7.78 (m, 1H), 7.82-7.97 (m, 1H), 8.09 (d, J=11.4 Hz, 2H), 8.60-8.90 (m, 1H), 10.33 (s, 1H).

### Example 104—Synthesis of 1-(5-(N-(1-Methyl-1H-Indazol-7-yl)Sulfamoyl)Pyridin-2-yl)-1H-Pyrazole-4-Carboxamide (I-105)

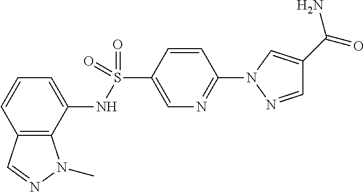 [SMILES: Cn1ncc2cccc(NS(=O)(=O)c3ccc(-n4cc(C(N)=O)cn4)nc3)c21]

The title compound was obtained using general procedure B as a white solid. (19 mg, 7%). (ES, m/z): [M+H]⁺ 398; ¹H NMR (400 MHz, DMSO-d₆) δ 4.30 (s, 3H), 6.54 (d, J=7.3 Hz, 1H), 6.93 (t, J=7.7 Hz, 1H), 7.31 (s, 1H), 7.66 (d, J=8.0 Hz, 1H), 7.88 (s, 1H), 8.08 (s, 1H), 8.15 (d, J=8.7 Hz, 1H), 8.22 (s, 1H), 8.27 (dd, J=2.3, 8.6 Hz, 1H), 8.65 (d, J=2.3 Hz, 1H), 9.22 (s, 1H), 10.32 (s, 1H).

### Example 105—Synthesis of 1-Methyl-N-(6-(4-(Trifluoromethyl)-1H-Pyrazol-1-yl)Pyridin-3-yl)-1H-Indazole-7-Sulfonamide (II-18)

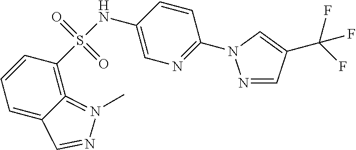

The title compound was obtained using general procedure A as an off-white solid (63 mg, 17%). (ES, m/z): [M+H]⁺ 423; ¹H NMR (400 MHz, CDCl₃) δ 4.37 (s, 3H), 7.11 (dd, J=8.4, 7.2 Hz, 1H), 7.64-7.96 (m, 6H), 7.99 (d, J=2.4 Hz, 1H), 8.09 (s, 1H), 8.54-8.72 (m, 1H).

### Example 106—Synthesis of N-(1-Methyl-1H-Indazol-7-yl)-6-(3-Methyl-1H-Pyrazol-1-yl)Pyridine-3-Sulfonamide (I-254)

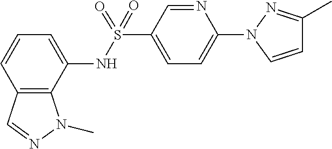 [SMILES: Cc1ccn(-c2ccc(S(=O)(=O)Nc3cccc4cnn(C)c34)cn2)n1]

The title compound was obtained using general procedure B as an off-white solid (29 mg, 16%). (ES, m/z): [M+H]⁺ 369; ¹H NMR (400 MHz, DMSO-d₆) δ 2.32 (s, 3H), 4.29 (s, 3H), 6.48 (d, J=2.6 Hz, 1H), 6.52 (d, J=7.2 Hz, 1H), 6.93 (t, J=7.6 Hz, 1H), 7.67 (d, J=8.0 Hz, 1H), 8.03 (d, J=8.4 Hz, 1H), 8.08 (s, 1H), 8.21-8.14 (m, 1H), 8.57 (t, J=2.0 Hz, 2H), 10.25 (s, 1H).

### Example 107—Synthesis of N-(6-Methoxy-1-Methyl-1H-Indazol-7-yl)-6-(3-Methyl-1H-1,2,4-Triazol-1-yl)Pyridine-3-Sulfonamide (I-255

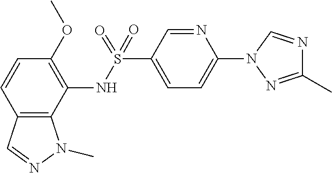 [SMILES: COc1ccc2cnn(C)c2c1NS(=O)(=O)c1ccc(-n2cnc(C)n2)nc1]

The title compound was obtained using general procedure B as an off-white solid (45 mg, 34%). (ES, m/z): [M+H]⁺ 400; ¹H NMR (400 MHz, DMSO-d₆) δ 2.42 (s, 3H), 3.17 (s, 3H), 4.28 (s, 3H), 6.80 (d, J=8.9 Hz, 1H), 7.60 (d, J=8.8 Hz, 1H), 7.97 (d, J=7.8 Hz, 2H), 8.24 (dd, J=2.3, 8.5 Hz, 1H), 8.66 (d, J=2.3 Hz, 1H), 9.34 (s, 1H).

### Example 108—Synthesis of N-(1-Methyl-1H-Indazol-7-yl)-6-(5-Methyl-1H-Pyrazol-1-yl)Pyridine-3-Sulfonamide (I-256)

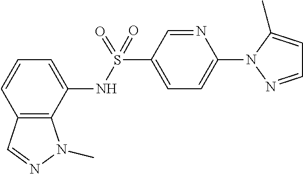 [SMILES: Cc1ccnn1-c1ccc(S(=O)(=O)Nc2cccc3cnn(C)c23)cn1]

The title compound was obtained using general procedure B as an off-white solid (2 mg, 1%). (ES, m/z): [M+H]⁺ 369; ¹H NMR (400 MHz, DMSO-d₆) δ 2.64 (s, 3H), 4.34 (s, 3H), 6.36 (s, 1H), 6.66 (d, J=7.6 Hz, 1H), 6.82 (t, J=7.6 Hz, 1H), 7.32 (s, 1H), 7.69 (d, J=1.6 Hz, 1H), 8.05-7.88 (m, 2H), 8.23-8.16 (m, 1H), 8.65 (d, J=2.4 Hz, 1H), 10.25 (s, 1H).

### Example 109—Synthesis of N-(1-Methyl-1H-Indazol-7-yl)-6-(5-(Trifluoromethyl)-1H-Pyrazol-1-yl)Pyridine-3-Sulfonamide (I-257)

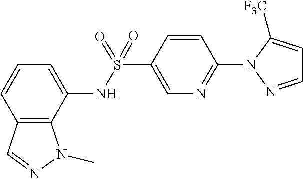 [SMILES: Cn1ncc2cccc(NS(=O)(=O)c3ccc(-n4nccc4C(F)(F)F)nc3)c21]

The title compound was obtained using general procedure B as an off-white solid (44 mg, 17%). (ES, m/z): [M+H]⁺ 423; ¹H NMR (400 MHz, DMSO-d₆) δ 4.29 (s, 3H), 6.52 (dd, J=1.0, 7.3 Hz, 1H), 6.93 (t, J=7.7 Hz, 1H), 7.17 (d, J=2.8 Hz, 1H), 7.71 (dd, J=1.0, 8.1 Hz, 1H), 8.11 (s, 1H), 8.17-8.25 (m, 1H), 8.30 (dd, J=2.4, 8.7 Hz, 1H), 8.59-8.73 (m, 1H), 8.94 (dd, J=1.1, 2.7 Hz, 1H), 10.39 (s, 1H).

### Example 110—Synthesis of N-(1-Methyl-1H-Indazol-7-yl)-6-(4-Methyl-1H-Pyrazol-1-yl)Pyridine-3-Sulfonamide (I-258)

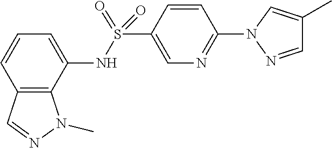 [SMILES: Cc1cnn(-c2ccc(S(=O)(=O)Nc3cccc4cnn(C)c34)cn2)c1]

The title compound was obtained using general procedure B as a white solid (50 mg, 22%). (ES, m/z): [M+H]⁺ 369; ¹H NMR (400 MHz, DMSO-d₆) δ 2.13 (s, 3H), 4.29 (s, 3H), 6.51 (dd, J=1.0, 7.3 Hz, 1H), 6.94 (dd, J=7.3, 8.1 Hz, 1H), 7.69-7.78 (m, 2H), 8.05-8.22 (m, 3H), 8.46-8.51 (m, 1H), 8.57 (dd, J=0.8, 2.4 Hz, 1H), 10.27 (s, 1H).

### Example 111—Synthesis of N-(6-Ethyl-1-Methyl-1H-Indazol-7-yl)-6-(5-Methyl-1H-Pyrazol-1-yl)Pyridine-3-Sulfonamide (I-259)

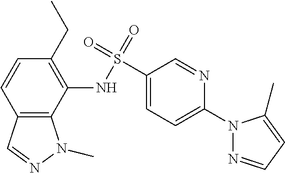 [SMILES: CCc1ccc2cnn(C)c2c1NS(=O)(=O)c1ccc(-n2nccc2C)nc1]

The title compound was obtained using general procedure B as an off-white solid (2 mg, 1%). (ES, m/z): [M+H]⁺ 397; ¹H NMR (400 MHz, DMSO-d₆) δ 0.80 (t, J=7.6 Hz, 3H), 2.05-2.07 (m, 2H), 2.65 (s, 3H), 4.25 (s, 3H), 6.40 (s, 1H), 6.96 (d, J=8.4 Hz, 1H), 7.61 (d, J=8.4 Hz, 1H), 7.74 (s, 1H), 7.99 (s, 1H), 8.07 (d, J=8.8 Hz, 1H), 8.11-8.25 (m, 1H), 8.35-8.75 (m, 1H), 10.35 (s, 1H).

### Example 112—Synthesis of 6′-Methyl-N-(1-Methyl-1H-Indazol-7-yl)-[2,3′-Bipyridine]-5-Sulfonamide (I-122)

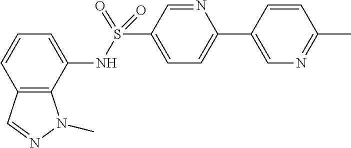 [SMILES: Cc1ccc(-c2ccc(S(=O)(=O)Nc3cccc4cnn(C)c34)cn2)cn1]

The title compound was obtained using general procedure D as a white solid (10 mg, 8%). (ES, m/z): [M+H]⁺ 380; ¹H NMR (400 MHz, DMSO-d₆) δ 2.55 (d, J=5.3 Hz, 3H), 4.30 (s, 3H), 6.56 (d, J=7.2 Hz, 1H), 6.91 (t, J=7.6 Hz, 1H), 7.43 (d, J=8.0 Hz, 1H), 7.61 (d, J=8.0 Hz, 1H), 8.06 (s, 1H), 8.17-8.09 (m, 1H), 8.25 (d, J=8.4 Hz, 1H), 8.47-8.36 (m, 1H), 8.87 (d, J=2.4 Hz, 1H), 9.21 (d, J=2.4 Hz, 1H), 10.34 (s, 1H).

EXAMPLE 113—SYNTHESIS OF N-(1-METHYL-1H-INDAZOL-7-YL)-[2,4′-BIPYRIDINE]-5-SULFONAMIDE (I-116)

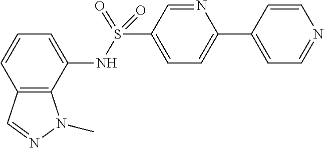 [SMILES: Cn1ncc2cccc(NS(=O)(=O)c3ccc(-c4ccncc4)nc3)c21]

The title compound was obtained using general procedure D as a light yellow solid (72 mg, 64%). (ES, m/z): [M+H]⁺ 366.1, ¹H NMR (400 MHz, DMSO-d₆) δ 4.30 (s, 3H), 6.55 (d, J=7.3 Hz, 1H), 6.89 (t, J=7.7 Hz, 1H), 7.59 (s, 1H), 8.05 (s, 1H), 8.15-8.09 (m, 2H), 8.19 (dd, J=8.4, 2.4 Hz, 1H), 8.34 (d, J=8.4 Hz, 1H), 8.79-8.73 (m, 2H), 8.93 (dd, J=2.4, 0.8 Hz, 1H), 10.40 (s, 1H).

### Exampel 114—Synthesis of N-(1-Methyl-1H-Indazol-7-yl)-6-(3-Methyl-4H-1,2,4-Triazol-4-yl)Pyridine-3-Sulfonamide (I-260)

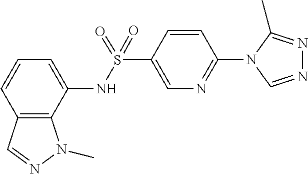 [SMILES: Cc1nncn1-c1ccc(S(=O)(=O)Nc2cccc3cnn(C)c23)cn1]

The title compound was obtained using general procedure B as an off-white solid (7 mg, 3%). (ES, m/z): [M+H]⁺ 370; ¹H NMR (400 MHz, DMSO-d₆) δ 2.82 (s, 3H), 4.30 (s, 3H), 6.54 (d, J=7.2 Hz, 1H), 6.95 (t, J=7.7 Hz, 1H), 7.71 (d, J=8.0 Hz, 1H), 8.06-8.14 (m, 2H), 8.18 (s, 1H), 8.29 (dd, J=2.5, 8.7 Hz, 1H), 8.72 (d, J=2.4 Hz, 1H), 10.40 (s, 1H).

### Example 115—Synthesis of 6-(3-Methoxy-1H-Pyrazol-1-yl)-N-(1-Methyl-1H-Indazol-7-yl) Pyridine-3-Sulfonamide (I-261)

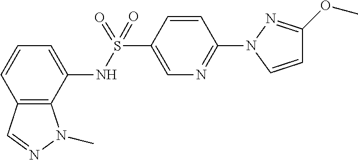 [SMILES: COc1ccn(-c2ccc(S(=O)(=O)Nc3cccc4cnn(C)c34)cn2)n1]

The title compound was obtained using general procedure B as an off-white solid (53 mg, 22%). (ES, m/z): [M+H]⁺ 385; ¹H NMR (400 MHz, DMSO-d₆) δ 3.95 (s, 3H), 4.29 (s, 3H), 6.21 (d, J=2.9 Hz, 1H), 6.53 (dd, J=7.4, 1.0 Hz, 1H), 6.94 (t, J=7.7 Hz, 1H), 7.69 (d, J=8.0 Hz, 1H), 7.90 (d, J=8.7 Hz, 1H), 8.09 (s, 1H), 8.16 (dd, J=8.7, 2.4 Hz, 1H), 8.54 (d, J=2.8 Hz, 2H), 10.23 (s, 1H).

### Example 116—Synthesis of N-(6-Methoxy-1-Methyl-1H-Benzo[D][1,2,3]Triazol-7-yl)-6-(4-(Trifluoromethyl)-1H-Pyrazol-1-yl)Pyridine-3-Sulfonamide (I-262)

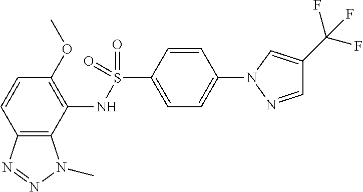 [SMILES: COc1ccc2nnn(C)c2c1NS(=O)(=O)c1ccc(-n2cc(C(F)(F)F)cn2)cc1]

The title compound was obtained using general procedure B as a white solid (33 mg, 17%). (ES, m/z): [M+H]⁺ 454; ¹H NMR (400 MHz, DMSO-d₆) δ 3.35 (s, 3H), 4.48 (s, 3H), 7.11 (d, J=9.2 Hz, 1H), 7.99(d J=9.2 Hz, 1H),8.16 (d, J=8.8 Hz, 1H), 8.28 (dd, J=8.4, 2.0 Hz, 1H), 8.42 (s, 1H), 8.68 (dd, J=4.8, 0.8 Hz, 1H), 9.34 (s, 1H), 10.40 (s, 1H).

### Example 117—Synthesis of N-(1-Methyl-1H-Indazol-7-yl)-6′-Oxo-1′,6′-Dihydro-[2,2′-Bipyridine]-5-Sulfonamide (I-118)

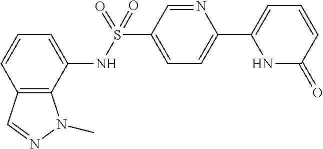 [SMILES: Cn1ncc2cccc(NS(=O)(=O)c3ccc(-c4cccc(=O)[nH]4)nc3)c21]

The title compound was obtained using general procedure D as a white solid (20 mg, 17%). (ES, m/z): [M+H]⁺ 382; ¹H NMR (400 MHz, DMSO-d₆) δ 4.29 (s, 3H), 6.49-6.75 (m, 2H), 6.89 (t, J=7.6 Hz, 1H), 7.60-7.70 (m, 3H), 8.04 (s, 1H), 8.17 (dd, J=8.4, 2.4 Hz, 1H), 8.34 (s, 1H), 8.86 (d, J=2.4 Hz, 1H), 10.38 (s, 1H), 11.16 (s, 1H).

### Example 118—Synthesis of 4′-Methyl-N-(1-Methyl-1H-Indazol-7-yl)-2,3′-Bipyridine-5-Sulfonamide (I-123)

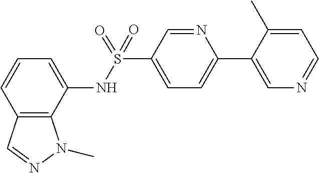 [SMILES: Cc1ccncc1-c1ccc(S(=O)(=O)Nc2cccc3cnn(C)c23)cn1]

The title compound was obtained using general procedure D as a white solid (55 mg, 47%) as a white solid. (ES, m/z): [M+H]⁺ 380; ¹H NMR (400 MHz, DMSO-d₆) δ 2.40 (s, 3H), 4.27 (s, 3H), 6.54 (dd, J=7.6, 1.0 Hz, 1H), 6.96 (t, J=7.6 Hz, 1H), 7.41 (d, J=5.2 Hz, 1H), 7.72 (d, J=7.6 Hz, 1H), 7.91 (d, J=8.4 Hz, 1H), 8.09-8.18 (m, 2H), 8.54 (d, J=5.0 Hz, 1H), 8.65 (s, 1H), 8.87 (d, J=2.4 Hz, 1H), 10.37 (s, 1H).

### Example 119—Synthesis of 6-[4-Methyl-3-(Trifluoromethyl) Pyrazol-1-Yl]-N-(1-Methylindazol-7-yl)Pyridine-3-Sulfonamide (I-114)

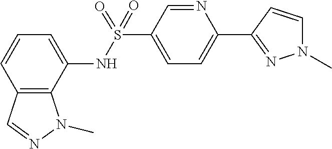 [SMILES: Cn1ccc(-c2ccc(S(=O)(=O)Nc3cccc4cnn(C)c34)cn2)n1]

The title compound was obtained using general procedure D as a white solid (40 mg, 34%). (ES, m/z): [M+H]⁺ 369; ¹H NMR (400 MHz, DMSO-d₆) δ 3.96 (s, 3H), 4.27 (s, 3H), 6.51 (d, J=6.8 Hz, 1H), 6.78-7.06 (m, 2H), 7.67 (d, J=8.0 Hz, 1H), 7.85 (d, J=2.4 Hz, 1H), 7.95-8.21 (m, 3H), 8.72 (d, J=1.6 Hz, 1H), 10.25 (s, 1H).

### Example 120—Synthesis of 3′-Fluoro-N-(1-Methylindazol-7-yl)-[2,4′-Bipyridine]-5-Sulfonamide (I-120)

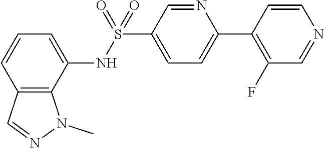 [SMILES: Cn1ncc2cccc(NS(=O)(=O)c3ccc(-c4ccncc4F)nc3)c21]

The title compound was obtained using general procedure D as a white solid (78 mg, 65%). (ES, m/z): [M+H]⁺ 384.1, ¹H NMR (400 MHz, DMSO-d₆) δ 4.29 (s, 3H), 6.55 (d, J=7.2 Hz, 1H), 6.93 (t, J=7.6 Hz, 1H), 7.65 (d, J=7.6 Hz, 1H), 8.00-8.11 (m, 2H), 8.17 (d, J=8.4 Hz, 1H), 8.25 (dd, J=8.4, 2.4 Hz, 1H), 8.63 (dd, J=4.8, 1.2 Hz, 1H), 8.79 (d, J=2.8 Hz, 1H), 8.97 (dd, J=2.4, 0.8 Hz, 1H), 10.46 (s, 1H).

### Example 121—Synthesis of 6-(3-Methoxy-1-Methyl-1H-Pyrazol-4-yl)-N-(1-Methyl-1H-Indazol-7-yl)Pyridine-3-Sulfonamide (I-125

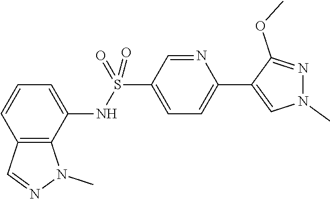 [SMILES: COc1nn(C)cc1-c1ccc(S(=O)(=O)Nc2cccc3cnn(C)c23)cn1]

The title compound was obtained using general procedure D as a white solid (45 mg, 36%) as an off-white solid. (ES, m/z): [M+H]⁺399; ¹H NMR (400 MHz, DMSO-d₆) δ 3.77 (s, 3H), 3.96 (s, 3H), 4.27 (s, 3H), 6.50 (d, J=7.6 Hz, 1H), 6.92 (t, J=7.6 Hz, 1H), 7.53-7.72 (m, 1H), 7.82 (d, J=8.4 Hz, 1H), 7.97 (dd, J=8.4, 2.4 Hz, 1H), 8.07 (s, 1H), 8.25 (s, 1H), 8.60 (d, J=2.0 Hz, 1H), 10.14 (s, 1H).

### Example 122—Synthesis of 2′-Methyl-N-(1-Methylindazol-7-yl)-[2,3′-Bipyridine]-5-Sulfonamide (I-113)

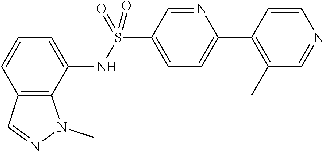 [SMILES: Cc1cnccc1-c1ccc(S(=O)(=O)Nc2cccc3cnn(C)c23)cn1]

The title compound was obtained using general procedure D as an off-white solid (52 mg, 40%). (ES, m/z): [M+H]⁺ 380; ¹H NMR (400 MHz, DMSO-d₆) δ 2.54 (s, 3H), 4.27 (s, 3H), 6.54 (d, J=7.6 Hz, 1H), 6.95 (t, J=7.6 Hz, 1H), 7.40 (dd, J=7.6, 4.8 Hz, 1H), 7.71 (d, J=8.0 Hz, 1H), 7.97-7.81 (m, 2H), 8.10 (s, 1H), 8.14 (dd, J=8.0, 2.4 Hz, 1H), 8.57 (dd, J=4.8, 1.6 Hz, 1H), 8.86 (d, J=1.6 Hz, 1H), 10.36 (s, 1H).

### Example 123—Synthesis of 6-(1,4-Dimethyl-1H-Pyrazol-5-yl)-N-(1-Methyl-1H-Indazol-7-yl)Pyridine-3-Sulfonamide (I-124)

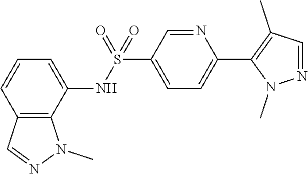 [SMILES: Cc1cnn(C)c1-c1ccc(S(=O)(=O)Nc2cccc3cnn(C)c23)cn1]

The title compound was obtained using general procedure D as a white solid (668 mg, 55%) as an off-white solid. (ES, m/z): [M+H]⁺ 383; ¹H NMR (400 MHz, DMSO-d₆) δ 2.15 (s, 3H), 3.95 (s, 3H), 4.26 (s, 3H), 6.53 (d, J=7.6 Hz, 1H), 6.95 (t, J=7.6 Hz, 1H), 7.42 (s, 1H), 7.71 (d, J=8.0 Hz, 1H), 7.82 (d, J=8.4 Hz, 1H), 7.99-8.22 (m, 2H), 8.86 (d, J=2.4 Hz, 1H), 10.36 (s, 1H).

### Example 124—Synthesis of 1′-Methyl-N-(1-Methyl-1H-Indazol-7-yl)-2′-Oxo-1′,2′-Dihydro-[2,4′-Bipyridine]-5-Sulfonamide (I-117)

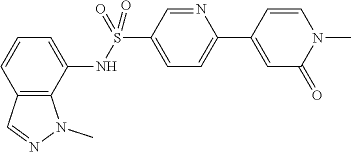 [SMILES: Cn1ccc(-c2ccc(S(=O)(=O)Nc3cccc4cnn(C)c34)cn2)cc1=O]

The title compound was obtained using general procedure D as a light yellow solid (22 mg, 18%). (ES, m/z): [M+H]⁺ 396; ¹H NMR (400 MHz, DMSO-d₆) δ 3.48 (s, 3H), 4.30 (s, 3H), 6.56 (d, J=7.3 Hz, 1H), 6.80-7.01 (m, 2H), 7.15 (s, 1H), 7.56 (d, J=8.1 Hz, 1H), 7.85 (d, J=7.1 Hz, 1H), 7.97-8.40 (m, 3H), 8.89 (s, 1H), 10.40 (s, 1H).

### Example 125—Synthesis of 1′-Methyl-N-(1-Methyl-1H-Indazol-7-yl)-6′-Oxo-1′,6′-Dihydro-[2,3′-Bipyridine]-5-Sulfonamide (I-115)

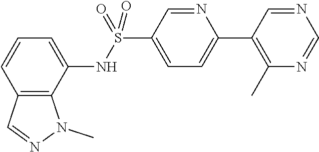 [SMILES: Cc1ncncc1-c1ccc(S(=O)(=O)Nc2cccc3cnn(C)c23)cn1]

The title compound was obtained using general procedure D as a white solid (23 mg, 24%) as a white solid. (ES, m/z): [M+H]⁺ 381; ¹H NMR (400 MHz, DMSO-d₆) δ 2.59 (s, 3H), 4.28 (s, 3H), 6.56 (d, J=7.2 Hz, 1H), 6.80-7.06 (m, 1H), 7.50-7.81 (m, 1H), 7.99 (d, J=8.0 Hz, 1H), 8.08 (s, 1H), 8.19 (dd, J=8.0, 2.4 Hz, 1H), 8.80-9.03 (m, 2H), 9.15 (s, 1H), 10.40 (s, 1H).

### Example 126—Synthesis of 5′-Chloro-N-(1-Methyl-1H-Indazol-7-yl)-[2,3′-Bipyridine]-5-Sulfonamide (I-121)

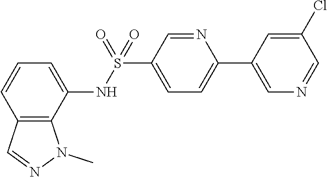 [SMILES: Cn1ncc2cccc(NS(=O)(=O)c3ccc(-c4cncc(Cl)c4)nc3)c21]

The title compound was obtained using general procedure D as a white solid (15 mg, 51%). (ES, m/z): [M+H]⁺ 400; ¹H NMR (400 MHz, DMSO-d₆) δ 4.29 (s, 3H), 6.53 (d, J=6.4 Hz, 1H), 6.92 (t, J=8.0 Hz, 1H), 7.67 (d, J=7.2 Hz, 1H), 8.09 (s, 1H), 8.17 (dd, J=8.0, 2.4 Hz, 1H), 8.39 (d, J=8.0 Hz, 1H), 8.65 (s, 1H), 8.79 (d, J=2.3 Hz, 1H), 8.91 (d, J=2.0 Hz, 1H), 9.13 (s, 1H), 10.39 (s, 1H).

### Example 127—Synthesis of N-(1-Methyl-1H-Indazol-7-yl)-6-(1-Methyl-1H-Pyrazol-4-yl)Pyridine-3-Sulfonamide (I-107)

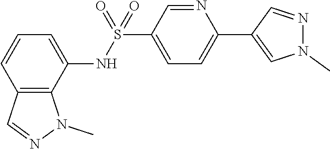 [SMILES: Cn1cc(-c2ccc(S(=O)(=O)Nc3cccc4cnn(C)c34)cn2)cn1]

The title compound was obtained using general procedure D as an off-white solid (70 mg, 31%). (ES, m/z): [M+H]⁺ 369; ¹H NMR (400 MHz, DMSO-d₆) δ 3.91 (s, 3H), 4.27 (s, 3H), 6.51 (d, J=7.3 Hz, 1H), 6.93 (t, J=7.7 Hz, 1H), 7.68 (d, J=8.0 Hz, 1H), 7.85 (d, J=8.4 Hz, 1H), 7.96 (dd, J=8.4, 2.4 Hz, 1H), 8.10 (m, 2H), 8.44 (s, 1H), 8.65 (d, J=2.3 Hz, 1H), 10.17 (s, 1H).

### Example 128—Synthesis of N-(1-Methyl-1H-Indazol-7-yl)-6-(2-Methylthiazol-5-yl)Pyridine-3-Sulfonamide (I-263)

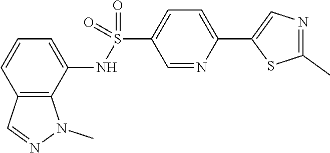 [SMILES: Cc1ncc(-c2ccc(S(=O)(=O)Nc3cccc4cnn(C)c34)cn2)s1]

The title compound was obtained using general procedure D as an off-white solid. (37 mg, 30%). (ES, m/z): [M+H]⁺ 386; ¹H NMR (400 MHz, DMSO-d₆) δ 2.72 (s, 3H), 4.29 (s, 3H), 6.52 (d, J=7.2 Hz, 1H), 6.94 (t, J=7.8 Hz, 1H), 7.70 (d, J=8.1 Hz, 1H), 7.99-8.14 (m, 2H), 8.19 (d, J=8.5 Hz, 1H), 8.52 (d, J=1.7 Hz, 1H), 8.72 (d, J=2.3 Hz, 1H), 10.31 (s, 1H).

### Example 129—Synthesis of N-(5-Methoxy-3-Methyl-1H-Indazol-4-yl)-6-(4-(Trifluoromethyl)-1H-Pyrazol-1-yl)Pyridine-3-Sulfonamide (I-106)

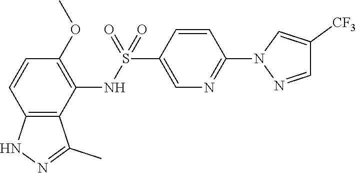 [SMILES: COc1ccc2[nH]nc(C)c2c1NS(=O)(=O)c1ccc(-n2cc(C(F)(F)F)cn2)nc1]

The title compound was obtained using general procedure A as a pink solid (38 mg, 45%). (ES, m/z): [M+H]⁺ 453; ¹H NMR (400 MHz, DMSO-d₆) δ 2.67 (s, 3H), 3.16 (s, 3H), 7.03 (d, J=9.1 Hz, 1H), 7.38 (d, J=8.9 Hz, 1H), 8.16 (d, J=8.6 Hz, 1H), 8.27 (dd, J=2.4, 8.7 Hz, 1H), 8.40 (s, 1H), 8.67 (d, J=2.3 Hz, 1H), 9.32 (t, J=1.1 Hz, 1H), 10.02 (s, 1H), 12.60 (s, 1H).

### Example 130—Synthesis of 2′-Methoxy-N-(1-Methyl-1H-Indazol-7-yl)-[2,4′-Bipyridine]-5-Sulfonamide (I-108)

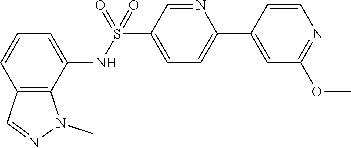 [SMILES: COc1cc(-c2ccc(S(=O)(=O)Nc3cccc4cnn(C)c34)cn2)ccn1]

The title compound was obtained using general procedure D as an off-white solid (698.5 mg, 28%). (ES, m/z): [M+H]⁺ 396; ¹H NMR (400 MHz, DMSO-d₆) δ 3.93 (s, 3H), 4.29 (s, 3H), 6.52 (dd, J=7.3, 1.0 Hz, 1H), 6.93 (t, J=7.7 Hz, 1H), 7.55 (t, J=1.1 Hz, 1H), 7.72 (m, 2H), 8.10 (s, 1H), 8.17 (dd, J=8.4, 2.4 Hz, 1H), 8.36 (m, 2H), 8.91 (d, J=2.3 Hz, 1H), 10.40 (s, 1H).

### Example 131—Synthesis of 1′-Methyl-N-(1-Methyl-1H-Indazol-7-yl)-6′-Oxo-1′,6′-Dihydro-[2,3′-Bipyridine]-5-Sulfonamide (I-112)

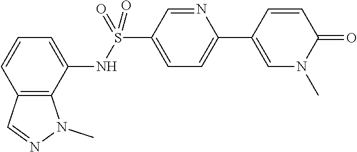 [SMILES: Cn1cc(-c2ccc(S(=O)(=O)Nc3cccc4cnn(C)c34)cn2)ccc1=O]

The title compound was obtained using general procedure B as a white solid (64 mg, 52%) as an off-white solid. (ES, m/z): [M+H]⁺ 396; ¹H NMR (400 MHz, DMSO-d₆) δ 3.56 (s, 3H), 4.28 (s, 3H), 6.39-6.66 (m, 2H), 6.80-7.04 (m, 1H), 7.64 (d, J=8.0 Hz, 1H), 8.02 (d, J=2.0 Hz, 2H), 8.07 (s, 1H), 8.22 (dd, J=9.6, 2.4 Hz, 1H), 8.72 (s, 2H), 10.24 (br s, 1H).

### Example 132—Synthesis of 6′-Methoxy-N-(1-Methyl-1H-Indazol-7-yl)-[2,2′-Bipyridine]-5-Sulfonamide (I-111)

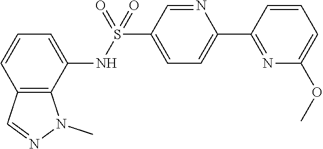 [SMILES: COc1cccc(-c2ccc(S(=O)(=O)Nc3cccc4cnn(C)c34)cn2)n1]

The title compound was obtained using general procedure D as a white solid (91 mg, 74%). (ES, m/z): [M+H]⁺ 396.1; ¹H NMR (400 MHz, DMSO-d₆) δ 4.01 (s, 3H), 4.29 (s, 3H), 6.51 (dd, J=7.3, 1.0 Hz, 1H), 6.93 (d, J=7.7 Hz, 1H), 7.00 (dd, J=8.3, 0.8 Hz, 1H), 7.70 (dd, J=8.1, 1.0 Hz, 1H), 7.92 (dd, J=8.2, 7.4 Hz, 1H), 8.15-8.03 (m, 2H), 8.20 (dd, J=8.4, 2.4 Hz, 1H), 8.628.51 (m, 1H), 8.91-8.77 (m, 1H), 10.36 (s, 1H).

### Example 133—Synthesis of 6-(2,5-Dimethylpyrazol-3-yl)-N-(1-Methyl-Indazol-7-yl)Pyridine-3-Sulfonamide (I-119)

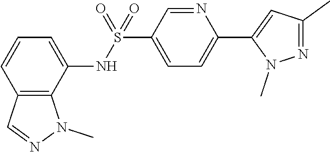 [SMILES: Cc1cc(-c2ccc(S(=O)(=O)Nc3cccc4cnn(C)c34)cn2)n(C)n1]

The title compound was obtained using general procedure D as a white solid (27 mg, 22%). (ES, m/z): [M+H]⁺ 383; ¹H NMR (400 MHz, DMSO-d₆) δ 2.21 (s, 3H), 4.10 (s, 3H), 4.28 (s, 3H), 6.50-6.60 (m, 2H), 6.77 (s, 1H), 6.94 (t, J=7.6 Hz, 1H), 7.69 (d, J=8.0 Hz, 1H), 7.98 (d, J=8.4 Hz, 1H), 8.08 (d, J=8.0 Hz, 1H), 8.81 (d, J=2.4 Hz, 1H), 10.33 (s, 1H).

### Example 134—Synthesis of N-(1-Methyl-1H-Indazol-7-yl)-[2,3′-Bipyridine]-5-Sulfonamide (I-109)

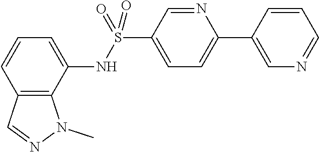 [SMILES: Cn1ncc2cccc(NS(=O)(=O)c3ccc(-c4cccnc4)nc3)c21]

The title compound was obtained using general procedure D as an off-white solid (12 mg, 11%). (ES, m/z): [M+H]⁺ 366; ¹H NMR (400 MHz, DMSO-d₆) δ 4.29 (s, 3H), 6.53 (d, J=7.2 Hz, 1H), 6.93 (t, J=7.7 Hz, 1H), 7.59 (dd, J=8.0, 4.8 Hz, 1H), 7.68 (d, J=8.1 Hz, 1H), 8.09 (s, 1H), 8.15 (dd, J=8.4, 2.4 Hz, 1H), 8.33 (d, J=8.4 Hz, 1H), 8.54 (dt, J=8.0, 2.0 Hz, 1H), 8.72 (dd, J=4.7, 1.6 Hz, 1H), 8.89 (d, J=2.3 Hz, 1H), 9.35 (d, J=2.3 Hz, 1H), 10.36 (s, 1H).

### Example 135—Synthesis of 6′-(Dimethylamino)-N-(1-Methyl-1H-Indazol-7-yl)-[2,3′-Bipyridine]-5-Sulfonamide (I-110)

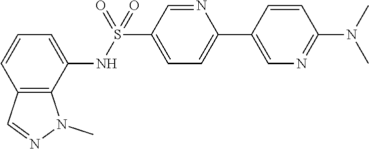 [SMILES: CN(C)c1ccc(-c2ccc(S(=O)(=O)Nc3cccc4cnn(C)c34)cn2)cn1]

The title compound was obtained using general procedure D as an off-white solid (6 mg, 5%). (ES, m/z): [M+H]⁺ 409; ¹H NMR (400 MHz, DMSO-d₆) δ 3.13 (s, 6H), 4.28 (s, 3H), 6.53 (dd, J=1.0, 7.4 Hz, 1H), 6.77 (d, J=9.0 Hz, 1H), 6.90-6.99 (m, 1H), 7.70 (dd, J=1.0, 8.1 Hz, 1H), 7.98 (dd, J=2.5, 8.5 Hz, 1H), 8.09 (d, J=6.6 Hz, 2H), 8.28 (dd, J=2.6, 9.1 Hz, 1H), 8.72 (d, J=2.4 Hz, 1H), 8.93 (d, J=2.5 Hz, 1H), 10.21 (s, 1H).

### Example 136—Synthesis of 6-(1-Ethoxyvinyl)-1-Methyl-7-Nitro-1H-Indazole

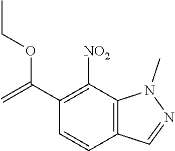 [SMILES: C=C(OCC)c1ccc2cnn(C)c2c1[N+](=O)[O-]]

To a stirred solution of 6-chloro-1-methyl-7-nitro-1H-indazole (500 mg, 2.4 mmol, 1.0 eq.) and tributyl(1-ethoxyvinyl)tin (0.96 mL, 2.8 mmol, 1.2 eq.) under nitrogen in 1,4-dioxane (12 mL) was added bis(triphenylphosphine)palladium(II) dichloride (166 mg, 0.2 mmol, 0.1 eq.) and stirred at 100° C. overnight. The reaction mixture was diluted with EtOAc (6 mL), washed with water (3×6 mL), dried over magnesium sulphate and concentrated under reduced pressure. The crude residue was purified by silica gel chromatography eluting with 0-50% EtOAc in cyclohexane to yield the title compound. ¹H NMR (400 MHz, DMSO-d₆) δ 1.24 (3H, dd, J=7.0, 7.0 Hz), 3.86 (2H, q, J=7.0 Hz), 3.92 (3H, s), 4.58 (1H, d, J=3.0 Hz), 4.66 (1H, d, J=3.0 Hz), 7.37 (1H, d, J=8.3 Hz), 8.06 (1H, d, J=8.4 Hz), 8.34 (1H, s).

### Example 137—Synthesis of 1-(7-Amino-1-Methyl-1H-Indazol-6-yl)Ethan-1-One

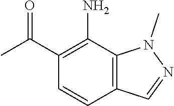

A stirred mixture of 1-(1-methyl-7-nitro-1H-indazol-6-yl)ethan-1-one (100 mg, 0.5 mmol, 1.0 eq.), ammonium chloride (122 mg, 2.3 mmol, 5.0 eq.) and iron powder (255 mg, 4.6 mmol, 10 eq.) in EtOH (6 mL) and water (2 mL) was stirred at 60° C. overnight. The reaction mixture was diluted with EtOAc (10 mL) and filtered through celite. The filter cake was washed with EtOAc (3×5 mL) and the solvent removed under reduced pressure to yield the title compound as an off-white solid (86 mg, 99%). LCMS: (ES, m/z): [M+H]⁺ 190.

### Example 138—Synthesis of 6-(Ethyl-1,1-D2)-1-Methyl-1H-Indazol-7-Amine

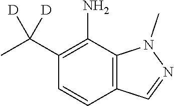

To a mixture of 1-(1-methyl-7-nitroindazol-6-yl)ethanone (1.0 g, 4.6 mmol, 1.0 eq.), AlCl₃ (3.65 g, 27 mmol, 6.0 eq.), in THF (50 mL) was added LiAlD₄ (1.15 g, 27 mmol, 6.0 eq.) at 0° C. under nitrogen. The resulting mixture was stirred for 16 h at 60° C. under nitrogen. The reaction was quenched by the addition of sat. NH₄Cl (20 mL) at 0° C., and the resulting mixture was extracted with EtOAc (3×30 mL). The combined organic layers were washed with brine (10 mL), dried over anhydrous Na₂SO₄, and concentrated under reduced pressure. The residue was purified by silica gel column chromatography, eluted with PE/EA (1:1) to afford the title compound (250 mg, 31%) as a yellow oil. LCMS: (ES, m/z): [M+H]⁺ 178.

### Example 139—Synthesis of N-(6-Methoxy-1-Methyl-1H-Pyrazolo[4,3-C]Pyridin-7-yl)-6-(3-(Trifluoromethyl)-1H-Pyrazol-5-yl)Pyridine-3-Sulfonamide (1-200)

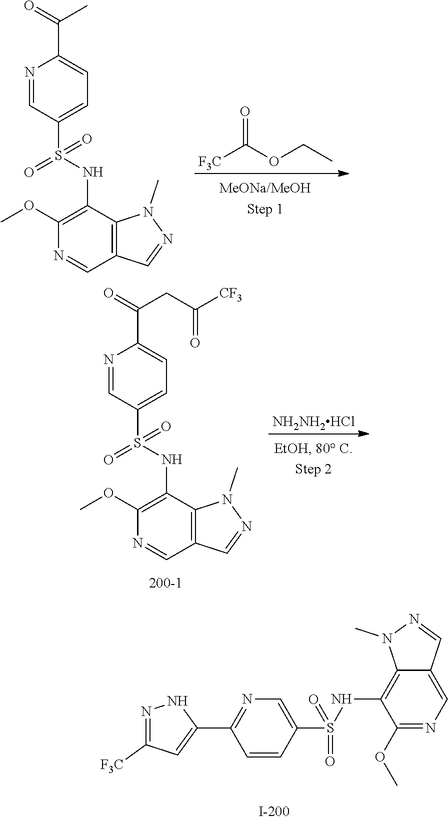

Step 1: Synthesis of 200-1. A solution of 6-acetyl-N-{6-methoxy-1-methylpyrazolo[4,3-c]pyridin-7-yl}pyridine-3-sulfonamide (293 mg, 0.811 mmol, 1 equiv.), MeONa (219.01 mg, 4.055 mmol, 5 equiv.) and trifluoroethyl acetate (230.39 mg, 1.622 mmol, 2 equiv.) in MeOH (5 mL) was stirred for 3 h at 80° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give N-{6-methoxy-1-methylpyrazolo[4,3-c]pyridin-7-yl}-6-(4,4,4-trifluoro-3-oxobutanoyl)pyridine-3-sulfonamide (222 mg, 53.88%) as a brown solid.

Step 2: Synthesis of N-(6-methoxy-1-methyl-1H-pyrazolo[4,3-c]pyridin-7-yl)-6-(3-(trifluoromethyl)-1H-pyrazol-5-yl)pyridine-3-sulfonamide (1-200). A solution of N-{6-methoxy-1-methylpyrazolo[4,3-c]pyridin-7-yl}-6-(4,4,4-trifluoro-3-oxobutanoyl)pyridine-3-sulfonamide (200 mg, 0.437 mmol, 1 equiv.) and NH₂NH₂. HCl (149.77 mg, 2.185 mmol, 5 equiv.) in EtOH (5 mL) was stirred overnight at 60° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (5% FA), 0% to 100% gradient in 20 min; detector: UV 254 nm, to give N-{6-methoxy-1-methylpyrazolo[4,3-c]pyridin-7-yl}-6-[5-(trifluoromethyl)-2H-pyrazol-3-yl]pyridine-3-sulfonamide (95.4 mg, 48.12%) as a white solid. LCMS: (ES, m/z): [M+H]⁺ 454; (400 MHz, DMSO-d₆) δ 3.33 (s, 3H), 4.24 (s, 3H), 7.57 (s, 1H), 8.16(d, J2.4 Hz, 2H), 8.24 (s, 1H). 8.66 (s, 1H), 8.81 (s, 1H), 10.22 (s, 1H), 14.64 (s, 1H)

### Example 140—Synthesis of N-(1-Methyl-3-Oxo-2,3-Dihydro-1H-Indazol-7-yl)-6-(4-(Trifluoromethyl)-1H-Pyrazol-1-yl)Pyridine-3-Sulfonamide (I-201)

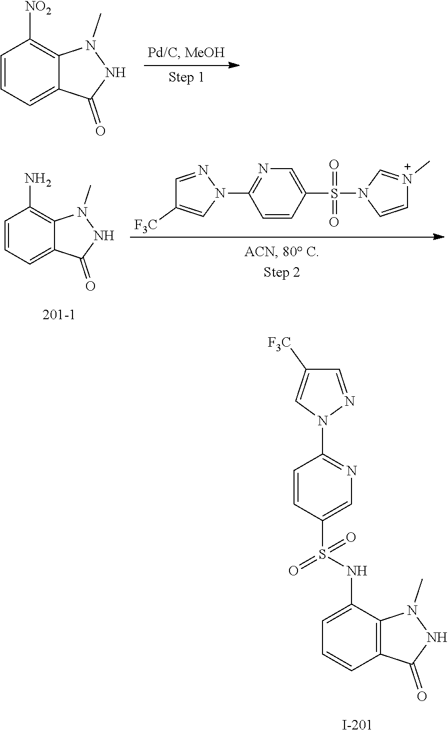 [SMILES: Cn1[nH]c(=O)c2cccc([N+](=O)[O-])c21 ; Cn1[nH]c(=O)c2cccc(N)c21 ; C[n+]1ccn(S(=O)(=O)c2ccc(-n3cc(C(F)(F)F)cn3)nc2)c1 ; Cn1[nH]c(=O)c2cccc(NS(=O)(=O)c3ccc(-n4cc(C(F)(F)F)cn4)nc3)c21]

Step 1: Synthesis of 201-1. A mixture of 1-methyl-7-nitro-2H-indazol-3-one (100 mg, 0.518 mmol, 1 equiv.) and Pd/C (50 mg, 0.470 mmol, 0.91 equiv.) in MeOH (7 mL) was stirred for 3 h at 80° C. The solid was filtrated. The filtration was concentrated under vacuum. This resulted in 201-1 (80 mg, 94.70%) as a purple semi-solid.

Step 2. Synthesis of N-(1-methyl-3-oxo-2,3-dihydro-1H-indazol-7-yl)-6-(4-(trifluoromethyl)-1H-pyrazol-1-yl)pyridine-3-sulfonamide (1-201). A solution of 201-1 (82.5 mg, 0.506 mmol, 1 equiv.) and 3-methyl-1-{6-[4-(trifluoromethyl)pyrazol-1-yl]pyridin-3-ylsulfonyl}imidazol-1-ium (271.74 mg, 0.759 mmol, 1.5 equiv.) in ACN (6 mL) was stirred overnight at 80° C. After the reaction was completed, the mixture was concentrated. The residue was purified by reversed-phase flash chromatography with the following conditions: column: C18 silica gel; mobile phase: ACN in water (10 mmol/L NH₄HCO₃), 0% to 100% gradient in 30 min; detector: UV 254 nm to give the crude product which was purified again by prep-HPLC with the following conditions: column: Xselect CSH C₁₈ OBD, 30*150 mm 5 μm; mobile phase A: water (0.1% FA), mobile phase B: ACN; flow rate 60 mL/min; gradient: 35% B to 51% B in 7 min; wavelength: 254 nm/220 nm; Rt1(min): 6.5) to afford N-(1-methyl-3-oxo-2,3-dihydro-1H-indazol-7-yl)-6-(4-(trifluoromethyl)-1H-pyrazol-1-yl)pyridine-3-sulfonamide (4.5 mg, 1.98%) as a purple solid. LCMS: (ES, m/z): [M+H]⁺ 439; (400 MHz, DMSO-d₆) δ 3.41 (s, 3H), 6.93-7.01 (m, 2H), 7.46 (d, J7.2 Hz, 1H), 8.15 (d, J8.8 Hz, 1H), 8.34-8.40 (m, 2H), 8.77 (d, J2.0 Hz, 1H), 9.28 (s, 1H), 10.07 (s, 1H), 10.31 (s, 1H).

### Example 141—Synthesis of 6-(3,4-Dimethyl-2-Oxo-2,3-Dihydro-1H-Imidazol-1-yl)-N-(6-Methoxy-1-Methyl-1H-Indazol-7-yl)Pyridine-3-Sulfonamide (I-202)

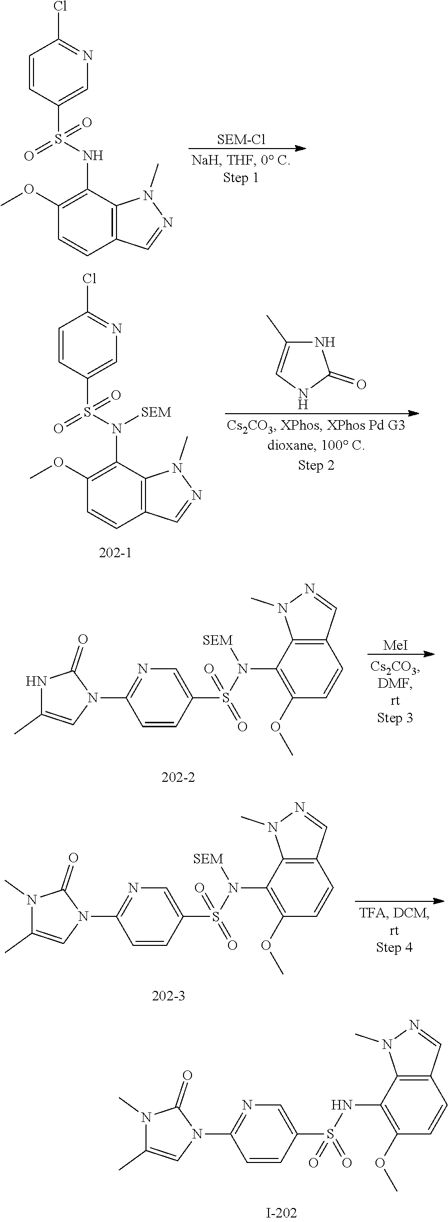

Step 1: Synthesis of 202-1. A solution of 6-chloro-N-(6-methoxy-1-methylindazol-7-yl)pyridine-3-sulfonamide (1 g, 2.835 mmol, 1 equiv.) in tetrahydrofuran (25 mL) was treated with NaH (0.14 g, 3.500 mmol, 1.23 equiv., 60%) for 10 min at 0° C. under a nitrogen atmosphere followed by the addition of [2-(chloromethoxy)ethyl]trimethylsilane (1.05 mL, 5.668 mmol, 2.00 equiv.) dropwise at 0° C. The resulting mixture was stirred for 14 h at room temperature under a nitrogen atmosphere. The resulting mixture was filtered and the filter cake was washed with EtOAc (3×25 mL). The filtrate was concentrated under reduced pressure. The residue was purified by reversed-phase flash chromatography with the following conditions: column: C18 silica gel; mobile phase: MeCN in water (10 mmol/L NH₄HCO₃), 90% to 95% gradient in 30 min; detector: UV 220 nm. This resulted in 202-1 (1 g, 73.03%) as a brown solid.

Step 2: Synthesis of 202-2. To a stirred mixture of 202-1 (140 mg, 0.290 mmol, 1 equiv.), Cs₂CO₃ (284 mg, 0.872 mmol, 3.01 equiv.), XPhos (27.6 mg, 0.058 mmol, 0.20 equiv.) and XPhos Pd G3 (7 mg, 0.008 mmol, 0.20 equiv.) in dioxane (2 mL) was added 4-methyl-1,3-dihydroimidazol-2-one (43 mg, 0.438 mmol, 1.51 equiv.) in portions at room temperature under a nitrogen atmosphere. The resulting mixture was stirred for 2 h at 100° C. under a nitrogen atmosphere. The resulting mixture was filtered and the filter cake was washed with MeCN (3×2 mL). The filtrate was concentrated under reduced pressure. The residue was purified by reversed-phase flash chromatography with the following conditions: column: C18 silica gel; mobile phase: MeCN in water (10 mmol/L NH₄HCO₃), 60% to 65% gradient in 10 min; detector: UV 210 nm. This resulted in 202-2 (45 mg, 28.51%) as a brown solid.

Step 3: Synthesis of 202-3. To a stirred solution of 202-2 (45 mg, 0.083 mmol, 1 equiv.) and Cs₂CO₃ (54 mg, 0.166 mmol, 2.01 equiv.) in DMF (1 mL) was added Mel (18 mg, 0.127 mmol, 1.54 equiv.) dropwise at room temperature under a nitrogen atmosphere. The resulting mixture was stirred for 2 h at room temperature under a nitrogen atmosphere. The resulting mixture was filtered and the filter cake was washed with MeCN (3×1 mL). The filtrate was concentrated under reduced pressure. The residue was purified by reversed-phase flash chromatography with the following conditions: column: C18 silica gel; mobile phase: MeCN in water (10 mmol/L NH₄HCO₃), 90% to 95% gradient in 30 min; detector: UV 220 nm. This resulted in 202-3 (38 mg, 82.31%) as a white solid.

Step 4: Synthesis of 6-(3,4-dimethyl-2-oxo-2,3-dihydro-1H-imidazol-1-yl)-N-(6-methoxy-1-methyl-1H-indazol-7-yl)pyridine-3-sulfonamide (I-202). To a stirred solution of 202-3 (38 mg, 0.068 mmol, 1 equiv.) in DCM (2.5 mL) was added TFA (2.5 mL, 33.658 mmol, 494.88 equiv.) dropwise at room temperature under a nitrogen atmosphere. The resulting mixture was stirred for 1 h at room temperature under a nitrogen atmosphere. The resulting mixture was concentrated under reduced pressure. The crude product (20 mg) was purified by prep-HPLC with the following conditions: column: YMC-Actus Triart C18 ExRS, 30*150 mm, 5 m; mobile phase A: water (10 mmol/L NH₄HCO₃+0.05% NH₃H₂O), mobile phase B: ACN; flow rate 60 mL/min; gradient: 22% B to 47% B in 7 min; wavelength: 220 nm; Rt1(min): 7.85; number of runs: 2) to afford 6-(3,4-dimethyl-2-oxo-2,3-dihydro-1H-imidazol-1-yl)-N-(6-methoxy-1-methyl-1H-indazol-7-yl)pyridine-3-sulfonamide as a white solid. LCMS: (ES, m/z): [M+H]⁺ 429; (400 MHz, DMSO-d₆): δ 2.11 (s, 3H), 3.15 (s, 3H), 3.17 (s, 3H), 4.26 (s, 3H), 6.81 (d, J9.2 Hz, 1H), 7.18 (d, J 1.2 Hz, 1H), 7.66 (d, J8.8 Hz, 1H), 7.98 (s, 1H), 8.10 (dd, J8.8, 2.4 Hz, 1H), 8.52 (s, 1H), 8.53 (d, J6.0 Hz, 1H), 9.85 (s, 1H).

### Example 142—Synthesis of 4-Amino-5-(4-Methoxybenzyl)-1-Methyl-2-(6-(4-(Trifluoromethyl)-1H-Pyrazol-1-yl)Pyridin-3-yl)Sulfonyl)-1,2-Dihydro-3H-Indazol-3-One (I-203)

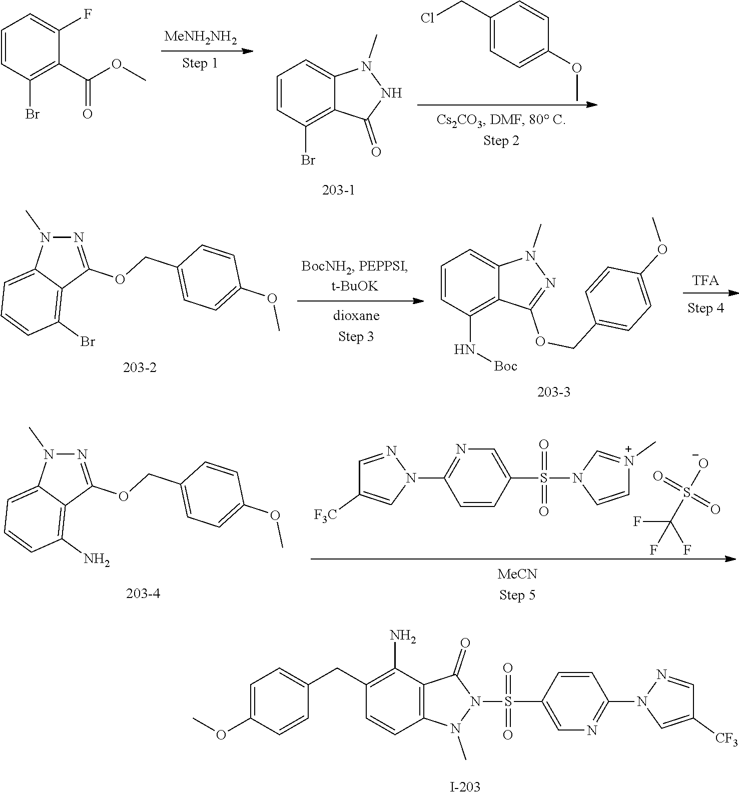

Step 1: Synthesis of 203-1. To a stirred solution of methyl 2-bromo-6-fluorobenzoate (3 g, 12.874 mmol, 1 equiv.) and methylhydrazine sulfuric acid salt (7.42 g, 51.496 mmol, 4 equiv.) in DMSO (60 mL) was added K₂CO₃ (10.68 g, 77.244 mmol, 6 equiv.) in portions at room temperature under a nitrogen atmosphere. The resulting mixture was stirred for 2 h at 100° C. under a nitrogen atmosphere. The precipitated solids were collected by filtration and washed with MeCN (3×100 mL). The filtrate was concentrated under reduced pressure. The resulting mixture was diluted with water (10 mL). The resulting mixture was extracted with EtOAc (3×200 mL). The combined organic layers were washed with water (3×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 40% to 50% gradient in 10 min; detector: UV 220 nm. The resulting mixture was concentrated under reduced pressure. This resulted in 4-bromo-1-methyl-2H-indazol-3-one (1.2 g, 41.05%) as a purple solid.

Step 2: Synthesis of 203-2. To a stirred solution of 4-bromo-1-methyl-2H-indazol-3-one (800 mg, 3.523 mmol, 1 equiv.) and Cs₂CO₃ (3443.86 mg, 10.569 mmol, 3 equiv.) in DMF (16 mL) was added 4-methoxybenzyl chloride (1103.56 mg, 7.046 mmol, 2 equiv.) dropwise at room temperature under a nitrogen atmosphere. The resulting mixture was stirred for 4 h at 80° C. under a nitrogen atmosphere. The reaction was monitored by LCMS. The precipitated solids were collected by filtration and washed with MeCN (3×50 mL). The filtrate was concentrated under reduced pressure. The resulting mixture was diluted with water (10 mL). The resulting mixture was extracted with EtOAc (3×50 mL). The combined organic layers were washed with water (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 45% to 47% gradient in 30 min; detector: UV 210 nm. This resulted in 4-bromo-3-[(4-methoxyphenyl)methoxy]-1-methylindazole (800 mg, 65.40%) as a white solid.

Step 3: Synthesis of 203-3. To a stirred solution of 4-bromo-3-[(4-methoxyphenyl)methoxy]-1-methylindazole (800 mg, 2.304 mmol, 1 equiv.), t-BuOK (9.7 mg, 0.086 mmol, 3.00 equiv.) and BocNH₂ (539.83 mg, 4.608 mmol, 2 equiv.) in dioxane (16 mL) was added Pd-PEPPSI-IPentCl 2-methylpyridine (o-picoline (2.42 mg, 0.003 mmol, 0.1 equiv.) in portions at room temperature under a nitrogen atmosphere. The resulting mixture was stirred for 21 h at 100° C. under a nitrogen atmosphere. The reaction was monitored by LCMS. The resulting mixture was diluted with water (10 mL). The resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (1 mmol/L FA), 60% to 65% gradient in 10 min; detector: UV 220 nm. This resulted in tert-butyl N-{3-[(4-methoxyphenyl)methoxy]-1-methylindazol-4-yl}carbamate (500 mg, 56.59%) as a yellow solid.

Step 4: Synthesis of 203-4. To a stirred solution of tert-butyl N-{3-[(4-methoxyphenyl)methoxy]-1-methylindazol-4-yl}carbamate (500 mg, 1.304 mmol, 1 equiv.) in DCM (10 mL) were added a mixture of TFA (1278.19 mg, 13.040 mmol, 10 equiv.) dropwise at room temperature under a nitrogen atmosphere. The resulting mixture was stirred for 2 h at room temperature under a nitrogen atmosphere. The reaction was monitored by LCMS. The resulting mixture was concentrated under reduced pressure. The residue was purified by reversed-phase flash chromatography with the following conditions: column: C18 silica gel; mobile phase: MeCN in water (10 mmol/L NH₄HCO₃), 48% to 50% gradient in 20 min; detector: UV 210 nm. This resulted in 3-[(4-methoxyphenyl)methoxy]-1-methylindazol-4-amine (120 mg, 32.48%) as a yellow solid.

Step 5: Synthesis of 4-amino-5-(4-methoxybenzyl)-1-methyl-2-(6-(4-(trifluoromethyl)-1H-pyrazol-1-yl)pyridin-3-yl)sulfonyl)-1,2-dihydro-3H-indazol-3-one (I-203). A solution/mixture of 4-amino-5-[(4-methoxyphenyl)methyl]-1-methyl-2H-indazol-3-one (100 mg, 0.353 mmol, 1 equiv.) and 1-methyl-3-{6-[4-(trifluoromethyl)pyrazol-1-yl]pyridin-3-ylsulfonyl}imidazol-1-ium triflate (214.89 mg, 0.424 mmol, 1.2 equiv.) in MeCN (10 mL) was stirred for 2 h at 50° C. under a nitrogen atmosphere. The resulting mixture was diluted with water (10 mL). The resulting mixture was extracted with EtOAc (3×50 mL). The combined organic layers were washed with brine (10 mL) and dried over anhydrous Na₂SO₄. After filtration, the filtrate was concentrated under reduced pressure. The crude product (100 mg) was purified by prep-HPLC with the following conditions: column: Xselect CSH C18 OBD, 30*150 mm 5 m; mobile phase A: water (0.1% FA), mobile phase B: ACN; flow rate 60 mL/min; gradient: 68% B to 78% B in 7 min; wavelength: 254 nm/220 nm; Rt1(min): 6.3) to afford 4-amino-5-[(4-methoxyphenyl)methyl]-1-methyl-2-{6-[4-(trifluoromethyl)pyrazol-1-yl]pyridin-3-ylsulfonyl}indazol-3-one (87 mg, 43.07%) as a yellow solid. LCMS: (ES, m/z): [M+H]⁺ 559; (400 MHz, DMSO-d₆) δ 3.69 (d, J 1.0 Hz, 3H), 3.77 (s, 3H), 3.80 (s, 2H), 5.11 (s, 2H), 6.70 (dd, J8.4 Hz, 1H), 6.78 (s, 2H), 7.01 (d, J8.8 Hz, 1H), 7.06 (d, J8.4 Hz, 2H), 8.24 (d, J8.8 Hz, 1H), 8.47 (s, 1H), 8.63 (dd, J8.8, 2.0 Hz, 1H), 9.07 (d, J2.4 Hz, 1H), 9.36 (s, 1H).

### Example 143—Synthesis of 6-(3-(Difluoromethyl)-1H-Pyrazol-5-yl)-N-(6-Methoxy-1-Methyl-1H-Pyrazolo[4,3-C]Pyridin-7-yl)Pyridine-3-Sulfonamide (1-204)

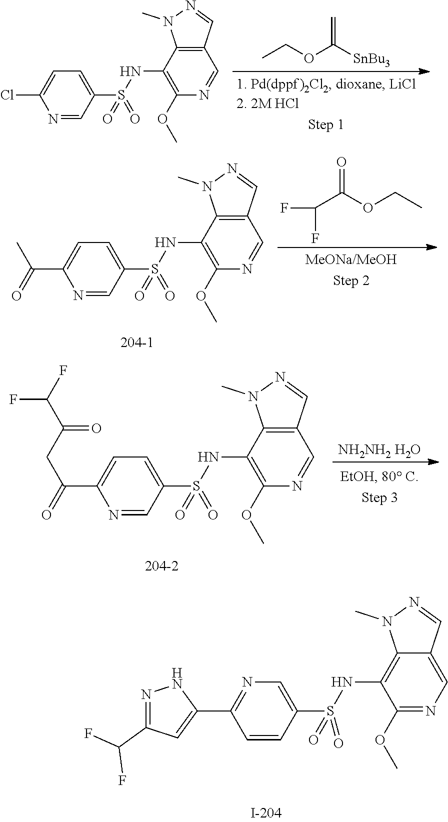

Step 1: Synthesis of 204-1. A solution of 6-chloro-N-{6-methoxy-1-methylpyrazolo[4,3-c]pyridin-7-yl}pyridine-3-sulfonamide (500 mg, 1.413 mmol, 1 equiv.), Pd(dppf)Cl2 (206.83 mg, 0.283 mmol, 0.2 equiv.), LiCl (119.82 mg, 2.826 mmol, 2 equiv.) and tributyl(1-ethoxyethenyl)stannane (1020.85 mg, 2.826 mmol, 2 equiv.) in 1,4-dioxane (10 mL) was stirred for 3 h at 100° C. under a nitrogen atmosphere. The mixture was treated with 2 M HCl (20 mL) for 30 min. The reaction was quenched by the addition of KF (aq.) (20 mL) at room temperature. The aqueous layer was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give 6-acetyl-N-{6-methoxy-1-methylpyrazolo[4,3-c]pyridin-7-yl}pyridine-3-sulfonamide (293 mg, 57.37%) as a brown yellow solid.

Step 2: Synthesis of 204-2. A solution of 6-acetyl-N-{6-methoxy-1-methylpyrazolo[4,3-c]pyridin-7-yl}pyridine-3-sulfonamide (100 mg, 0.277 mmol, 1 equiv.), MeONa (74.75 mg, 1.385 mmol, 5 equiv.) and ethyl 2,2-difluoroacetate (68.67 mg, 0.554 mmol, 2 equiv.) in MeOH (3 mL) was stirred for 3 h at 70° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give 6-(4,4-difluoro-3-oxobutanoyl)-N-{6-methoxy-1-methylpyrazolo[4,3-c]pyridin-7-yl}pyridine-3-sulfonamide (120 mg, 83.89%) as a brown solid.

Step 3: Synthesis of 6-(3-(difluoromethyl)-1H-pyrazol-5-yl)-N-(6-methoxy-1-methyl-1H-pyrazolo[4,3-c]pyridin-7-yl)pyridine-3-sulfonamide (I-204)

Step 3: Synthesis of 6-(3-(difluoromethyl)-1H-pyrazol-5-yl)-N-(6-methoxy-1-methyl-1H-pyrazolo[4,3-c]pyridin-7-yl)pyridine-3-sulfonamide (I-204). A solution of 6-(4,4-difluoro-3-oxobutanoyl)-N-{6-methoxy-1-methylpyrazolo[4,3-c]pyridin-7-yl}pyridine-3-sulfonamide (95 mg, 0.216 mmol, 1 equiv.) and NH₂NH₂. HCl (74.05 mg, 1.080 mmol, 5 equiv.) in EtOH (3 mL) was stirred overnight at 60° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: YMC-Actus Triant C18 ExRs, 30*150 mm, 5 m; mobile phase A: water(10 mmol/L NH₄HCO₃+0.05% NH3H₂O), mobile phase B: ACN; flow rate 60 mL/min; gradient: 5% B to 37% B in 8 min; wavelength: 220 nm; Rt1(min): 7.71) to afford 6-[5-(difluoromethyl)-2H-pyrazol-3-yl]-N-{6-methoxy-1-methylpyrazolo[4,3-c]pyridin-7-yl}pyridine-3-sulfonamide (12.6 mg, 13.14%) as an off-white solid. LCMS: (ES, m/z): [M+H]⁺ 436; (400 MHz, DMSO-d₆) δ 3.33 (s, 3H), 4.24 (s, 3H), 7.08(t, J54.4 Hz, 1H), 7.32 (s, 1H), 8.12(d, J8.8 Hz, 1H), 8.14(d, J8.8 Hz, 1H),8.24 (s, 1H), 8.66 (s, 1H), 8.81 (s, 1H), 10.22 (s, 1H), 14.64 (s, 1H).

### Example 144—Synthesis of 6-(3-Cyclopropyl-1H-Pyrazol-5-yl)-N-(6-Methoxy-1-Methyl-1H-Pyrazolo[4,3-C]Pyridin-7-yl)Pyridine-3-Sulfonamide (I-205)

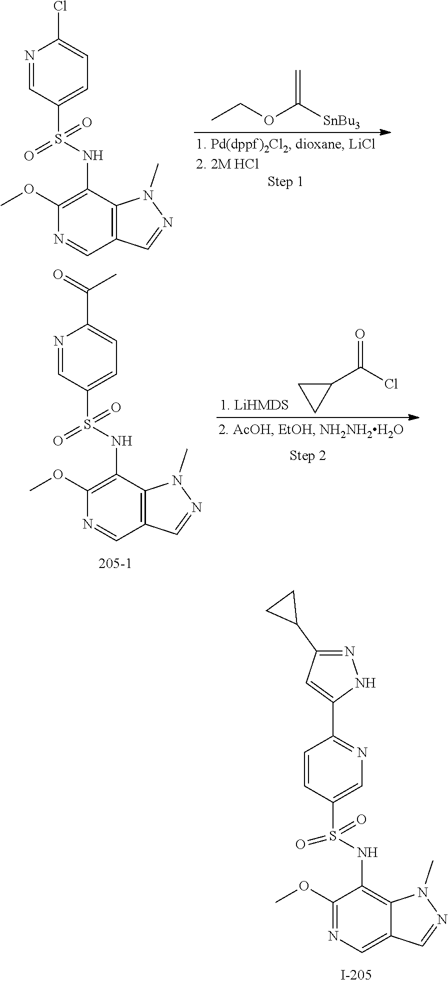

Step 1: Synthesis of 205-1. A solution of 6-chloro-N-{6-methoxy-1-methylpyrazolo[4,3-c]pyridin-7-yl}pyridine-3-sulfonamide (500 mg, 1.413 mmol, 1 equiv.), Pd(dppf)Cl2 (206.83 mg, 0.283 mmol, 0.2 equiv.), LiCl (119.82 mg, 2.826 mmol, 2 equiv.) and tributyl(1-ethoxyethenyl)stannane (1020.85 mg, 2.826 mmol, 2 equiv.) in1,4-dioxane (10 mL) was stirred for 3 h at 100° C. under a nitrogen atmosphere. The mixture was treated with 2 M HCl (20 mL) for 30 min. The reaction was quenched by the addition of KF (aq.) (20 mL) at room temperature. The aqueous layer was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give 6-acetyl-N-{6-methoxy-1-methylpyrazolo[4,3-c]pyridin-7-yl}pyridine-3-sulfonamide (293 mg, 57.37%) as a brown yellow solid.

Step 2: Synthesis of 6-(3-cyclopropyl-1H-pyrazol-5-yl)-N-(6-methoxy-1-methyl-1H-pyrazolo[4,3-c]pyridin-7-yl)pyridine-3-sulfonamide (1-205). A solution of 6-acetyl-N-{6-methoxy-1-methylpyrazolo[4,3-c]pyridin-7-yl}pyridine-3-sulfonamide (100 mg, 0.277 mmol, 1 equiv.) in toluene (1 mL) was treated with LiHMDS (92.61 mg, 0.554 mmol, 2 equiv.) for 5 min at 0° C. followed by the addition of cyclopropanecarbonyl chloride (28.93 mg, 0.277 mmol, 1 equiv.) dropwise at 0° C. the mixture was stirred for 3 h at 0° C. The ice bath was removed and AcOH (1.00 mL), EtOH (3 mL) and NH₂NH₂. H₂O (0.13 mL, 2.770 mmol, 10 equiv.) was added to the mixture under room temperature. The mixture was refluxed at 80° C. for 30 min. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by prep-HPLC with the following conditions: column: XBridge Shield RP18 OBD, 30*150 mm, 5 m; mobile phase A: water(10 mmol/L NH₄HCO₃+0.05% NH₃H₂O), mobile phase B: ACN; flow rate 60 mL/min; gradient: 8% B to 38% B in 7 min; wavelength: 254 nm/220 nm; Rt1(min): 7.32) to afford 6-(5-cyclopropyl-2H-pyrazol-3-yl)-N-{6-methoxy-1-methylpyrazolo[4,3-c]pyridin-7-yl}pyridine-3-sulfonamide (9.4 mg, 7.95%) as a white solid. LCMS: (ES, m/z): [M+H]⁺ 426; ¹H NMR (400 MHz, DMSO-d₆) δ 0.74-0.77 (m, 2H), 0.93-0.96 (m, 2H), 1.93-1.95 (m, 1H), 3.32 (s, 3H), 4.24 (s, 3H), 6.57 (s, 1H), 7.98(d, J2.8 Hz, 1H), 8.06 (d, J2.8 Hz, 1H), 8.20 (s, 1H), 8.60 (s, 1H), 8.67 (s, 1H), 10.04 (s, 1H), 13.04 (s, 1H)

### Example 145—Synthesis of N-(6-Methoxy-1-(Methyl-D3)-1H-Indazol-7-yl)-6-(4-Methyl-1H-Pyrazol-1-yl)Pyridine-3-Sulfonamide (I-206)

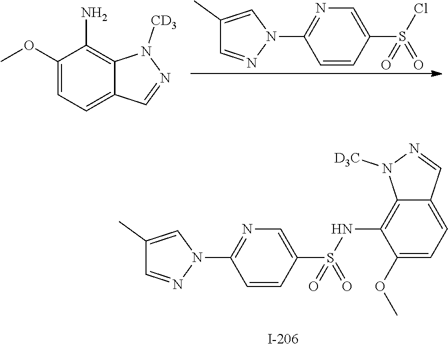

A solution of 6-methoxy-1-(methyl-d3)-1H-indazol-7-amine (100 mg, 0.555 mmol, 1 equiv.) and 6-(4-methylpyrazol-1-yl)pyridine-3-sulfonyl chloride (157.28 mg, 0.611 mmol, 1.1 equiv.) in pyridine (6 mL) was stirred for 4 h at room temperature. The resulting mixture was concentrated under reduced pressure and diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by prep-HPLC with the following conditions: column: XBridge Prep OBD C18, 30*150 mm, 5 μm; mobile phase A: water (10 mmol/L NH₄HCO₃+0.05% NH3. H₂O), mobile phase B: ACN; flow rate 60 mL/min; gradient: 32% B to 52% B in 7 min; wavelength: 254 nm/220 nm; Rt1(min): 7.4; number of runs: 2) to afford N-(6-methoxy-1-(methyl-d3)-1H-indazol-7-yl)-6-(4-methyl-1H-pyrazol-1-yl)pyridine-3-sulfonamide (12 mg, 5.37%) as an off-white solid. LCMS: (ES, m/z): [M+H]⁺ 402; (400 MHz, DMSO-d₆) δ 2.21(s, 3H), 3.17 (s, 3H), 6.82(d, J9.2 Hz, 1H), 7.67(d, J8.8 Hz, 1H), 7.75 (s, 1H), 7.99 (s, 1H), 8.04 (d, J 8.4 Hz, 1H),8.15(d, J2.4 Hz, 1H), 8.48 (s, 1H), 8.57(d, J2.0 Hz, 1H), 9.99 (s, 1H)

### Example 146—Synthesis of 6-(3-Cyclopropyl-1H-Pyrazol-1-yl)-N-(1-Methyl-1H-Indazol-7-yl)Pyridine-3-Sulfonamide (I-207)

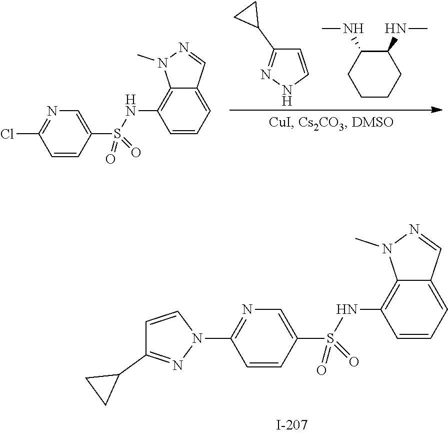

To a stirred solution of 6-chloro-N-(1-methylindazol-7-yl)pyridine-3-sulfonamide (100 mg, 0.310 mmol, 1 equiv.) and 3-cyclopropyl-1H-pyrazole (50.26 mg, 0.465 mmol, 1.5 equiv.) in DMSO (3 mL) was added 1,2-cyclohexanediamine (17.69 mg, 0.155 mmol, 0.5 equiv.), CuI (29.50 mg, 0.155 mmol, 0.5 equiv.) and Cs₂CO₃ (201.89 mg, 0.620 mmol, 2 equiv.) in portions at room temperature under a nitrogen atmosphere. The resulting mixture was stirred for 3 h at 120° C. under a nitrogen atmosphere. The mixture was allowed to cool down to room temperature. The residue was purified by reversed-phase flash chromatography with the following conditions: column: C18 silica gel; mobile phase: MeCN in water (0.1% FA), 10% to 50% gradient in 10 min; detector: UV 254 nm. The crude product (80 mg) was purified by prep-HPLC with the following conditions: column: XBridge Prep OBD C18, 30*150 mm, 5 m; mobile phase A: water (10 mmol/L NH₄HCO₃+0.05% NH3. H₂O), mobile phase B: ACN; flow rate 60 mL/min; gradient: 23% B to 50% B in 7 min; wavelength: 254 nm/220 nm; Rt1(min): 7.15) to afford 6-(3-cyclopropylpyrazol-1-yl)-N-(1-methylindazol-7-yl)pyridine-3-sulfonamide (26.5 mg, 21.68%) as an off-white solid. LCMS: (ES, m/z): [M+H]⁺ 395; ¹H NMR (400 MHz, DMSO-d₆) δ 0.80-0.83 (m, 2H), 0.97-1.01 (m, 2H), 2.01-2.04 (m, 1H), 4.29 (s, 3H), 6.40 (d, J2.4 Hz, 1H), 6.51 (d, J7.2 Hz, 1H), 6.93 (t, J7.6 Hz, 1H), 7.68 (d, J7.6 Hz, 1H), 8.01 (d, J8.8 Hz, 1H), 8.09 (s, 1H), 8.17 (dd, J8.8, 2.4 Hz, 1H), 8.55-8.56 (m, 2H), 10.25 (s, 1H).

### Example 147—Synthesis of 6-(4-Cyclopropyl-1H-Pyrazol-1-yl)-N-(1-Methyl-1H-Indazol-7-yl)Pyridine-3-Sulfonamide (I-208)

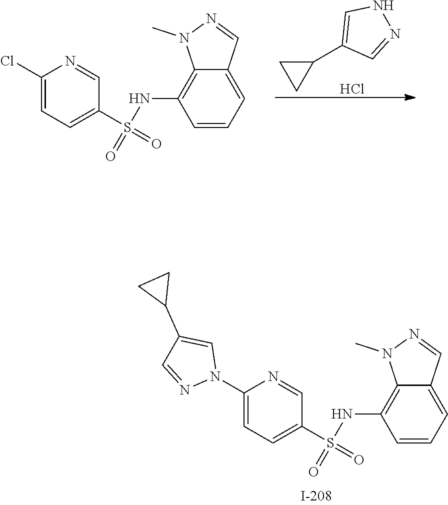 [SMILES: Cn1ncc2cccc(NS(=O)(=O)c3ccc(Cl)nc3)c21 ; c1n[nH]cc1C1CC1 ; Cl ; Cn1ncc2cccc(NS(=O)(=O)c3ccc(-n4cc(C5CC5)cn4)nc3)c21]

To a stirred solution of 4-cyclopropyl-1H-pyrazole hydrochloride (140 mg, 0.968 mmol, 1 equiv.) and 6-chloro-N-(1-methylindazol-7-yl) pyridine-3-sulfonamide (375.00 mg, 1.162 mmol, 1.2 equiv.) in DMSO (8 mL) were added Cs₂CO₃ (315.45 mg, 0.968 mmol, 1 equiv.), CuI (645.37 mg, 3.388 mmol, 3.5 equiv.) and (1R,2R)—N1,N2-dimethylcyclohexane-1,2-diamine (68.86 mg, 0.484 mmol, 0.5 equiv.) in portions at room temperature under a nitrogen atmosphere. The resulting mixture was stirred for 2 h at 120° C. under a nitrogen atmosphere. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. The resulting mixture was concentrated under reduced pressure. This resulted in 6-(4-cyclopropylpyrazol-1-yl)-N-(1-methylindazol-7-yl) pyridine-3-sulfonamide (58.8 mg, 15.40%) as a brown solid. LCMS: (ES, m/z): [M+H]⁺ 395; ¹H NMR (400 MHz, DMSO-d₆): δ 0.63-0.68 (m, 2H), 0.87-0.93 (m, 2H), 1.80-1.84 (m, 1H), 4.28 (s, 4H), 6.50 (d, J7.6 Hz, 1H), 6.94 (t, J7.6 Hz, 1H), 7.71 (d, J8.0 Hz, 1H), 7.78 (s, 1H), 8.05 (d, J8.8 Hz, 1H), 8.10 (s, 1H), 8.18 (d, J8.8 Hz, 1H), 8.44 (s, 1H), 8.55 (s, 1H), 10.25 (s, 1H).

### Example 148—Synthesis of N-(1-Methyl-1H-Indazol-7-yl)-6-(3-Methyl-2-Oxo-2,3-Dihydro-1H-Imidazol-1-yl)Pyridine-3-Sulfonamide (I-209)

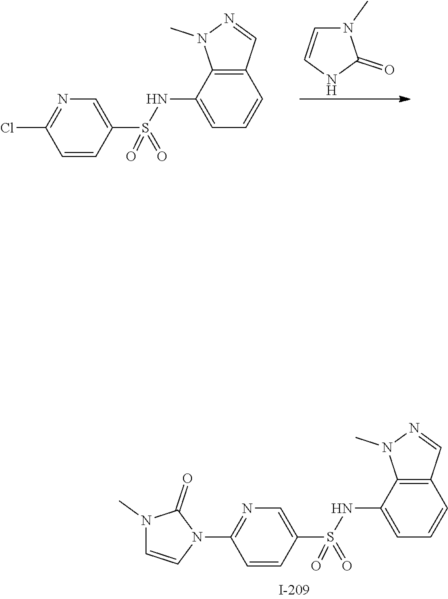

A mixture of 6-chloro-N-(1-methylindazol-7-yl)pyridine-3-sulfonamide (100 mg, 0.310 mmol, 1 equiv.), 1-methyl-3H-imidazol-2-one (50 mg, 0.510 mmol, 1.65 equiv.), Pd₂(dba)₃. CHCl₃ (60 mg, 0.058 mmol, 0.19 equiv.), XantPhos (40 mg, 0.069 mmol, 0.22 equiv.), KI (100 mg, 0.602 mmol, 1.94 equiv.) and Cs₂CO₃ (300 mg, 0.921 mmol, 2.97 equiv.) in 1,4-dioxane (5 mL) was stirred for 2 h at 100° C. under a nitrogen atmosphere. The resulting mixture was diluted with water (5 mL). The resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by silica gel column chromatography, eluted with CH₂Cl₂/MeOH (10:1) to afford N-(1-methyl-1H-indazol-7-yl)-6-(3-methyl-2-oxo-2,3-dihydro-1H-imidazol-1-yl)pyridine-3-sulfonamide (13.1 mg, 10.88%) as an off-white solid. LCMS: (ES, m/z): [M+H]⁺ 385; ¹H NMR (400 MHz, DMSO-d₆) δ 3.23 (s, 3H), 4.28 (s, 3H), 6.51 (dd, J7.8 Hz, 1H), 6.84 (d, J3.2 Hz, 1H), 6.93 (t, J7.6 Hz, 1H), 7.36 (d, J3.2 Hz, 1H), 7.67 (d, J7.6 Hz, 1H), 8.08 (s, 1H), 8.17 (dd, J8.8, 2.4 Hz, 1H), 8.53-8.63 (m, 2H), 10.20 (s, 1H).

### Example 149—Synthesis of 6′-Methoxy-N-(1-Methyl-1H-Indazol-7-yl)-[2,3′-Bipyridine]-5-Sulfonamide (I-210)

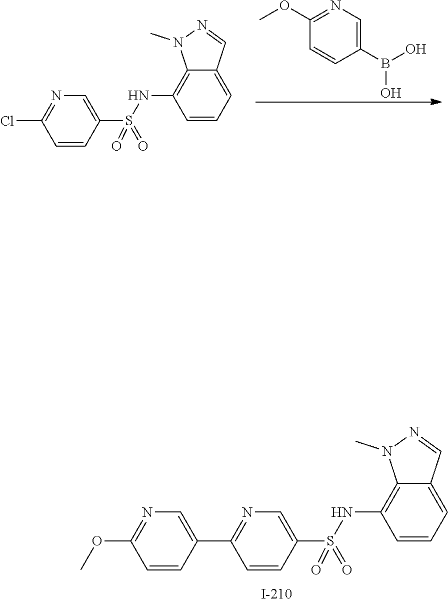

To a stirred solution of 6-chloro-N-(1-methylindazol-7-yl) pyridine-3-sulfonamide (120 mg, 0.372 mmol, 1 equiv.), 6-methoxypyridin-3-yl boronic acid (85.29 mg, 0.558 mmol, 1.5 equiv.) and Cs₂CO₃ (363.40 mg, 1.116 mmol, 3 equiv.) in dioxane (4 mL) and H₂O (1 mL) were added XPhos Pd G3 (62.94 mg, 0.074 mmol, 0.2 equiv.) and XPhos (35.45 mg, 0.074 mmol, 0.2 equiv.) at room temperature under a nitrogen atmosphere. The resulting mixture was stirred overnight at 100° C. The resulting mixture was diluted with water (20 mL). The resulting mixture was extracted with EtOAc (3×10 mL). The combined organic layers were washed with brine (3×10 mL) and dried over anhydrous Na₂SO₄. After filtration, the filtrate was concentrated under reduced pressure. The crude product (150 mg) was purified by prep-HPLC with the following conditions: column: XBridge Prep OBD C18, 30*150 mm, 5 m; mobile phase A: water (10 mmol/L NH₄HCO₃+0.05% NH₃·H₂O), mobile phase B: ACN; flow rate 60 mL/min; gradient: 10% B to 37% B in 7 min; wavelength: 254 nm/220 nm; Rt1(min): 8.57, to afford 6′-methoxy-N-(1-methylindazol-7-yl)-[2,3′-bipyridine]-5-sulfonamide (77.4 mg, 52.59%) as a white solid. LCMS: (ES, m/z): [M+H]⁺ 396; ¹H NMR (400 MHz, DMSO-d₆) δ 3.95 (s, 3H), 4.29 (s, 3H), 6.53 (dd, J 7.2, 0.9 Hz, 1H), 6.94 (t, J7.6 Hz, 1H), 7.00 (d, J 8.8 Hz, 1H), 7.70 (d, J 8.0 Hz, 1H),8.08 (d, J 2.4 Hz, 1H), 8.10 (s, 1H), 8.23 (d, J 8.4 Hz, 1H), 8.48 (dd, J 8.8, 2.4 Hz, 1H), 8.83 (d, J2.4 Hz, 1H), 9.00 (d, J2.4 Hz, 1H), 10.31 (s, 1H).

### Example 150—Synthesis of N-(6-Methoxy-1-Methyl-1H-Pyrazolo[4,3-C]Pyridin-7-yl)-6-(5-(Trifluoromethyl)-1H-Pyrazol-1-yl)Pyridine-3-Sulfonamide (I-211)

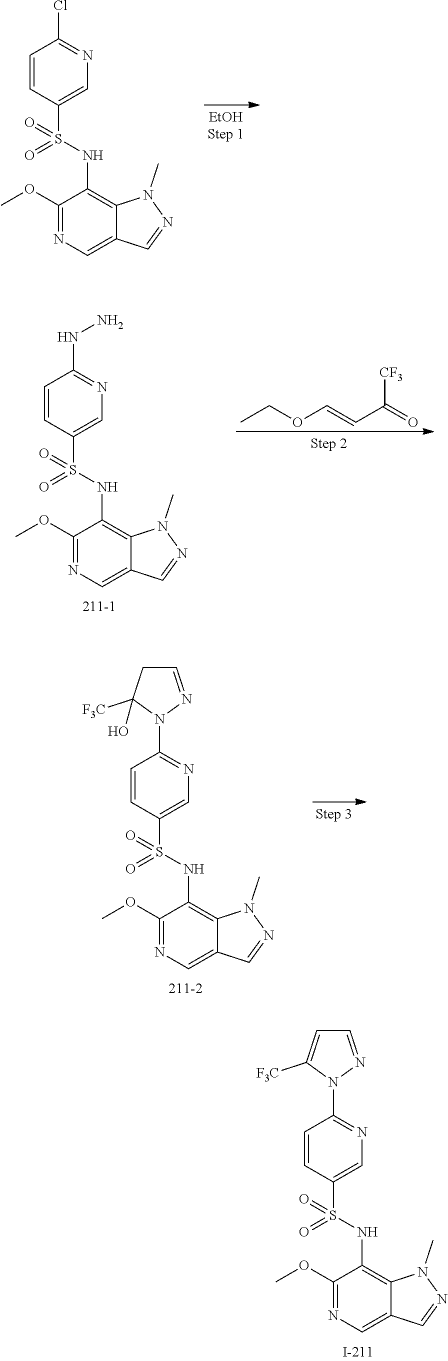 [SMILES: COc1ncc2cnn(C)c2c1NS(=O)(=O)c1ccc(Cl)nc1 ; COc1ncc2cnn(C)c2c1NS(=O)(=O)c1ccc(NN)nc1 ; CCO/C=C/C(=O)C(F)(F)F ; COc1ncc2cnn(C)c2c1NS(=O)(=O)c1ccc(N2N=CCC2(O)C(F)(F)F)nc1 ; COc1ncc2cnn(C)c2c1NS(=O)(=O)c1ccc(-n2nccc2C(F)(F)F)nc1]

Step 1: Synthesis of 211-1. A mixture of 6-chloro-N-(6-methoxy-1-methyl-1H-pyrazolo[4,3-c]pyridin-7-yl)pyridine-3-sulfonamide (200.00 mg, 0.565 mmol, 1 equiv.) and NH₂NH₂. H₂O (33.96 mg, 0.678 mmol, 1.2 equiv.) in EtOH (3 mL) was stirred for 16 h at 80° C. The resulting mixture was diluted with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 10% to 50% gradient in 10 min; detector: UV 254 nm, to afford 6-hydrazineyl-N-(6-methoxy-1-methyl-1H-pyrazolo[4,3-c]pyridin-7-yl)pyridine-3-sulfonamide (100 mg, 50.18%) as an off-white solid.

Step 2: Synthesis of 211-2. A mixture of 6-hydrazineyl-N-(6-methoxy-1-methyl-1H-pyrazolo[4,3-c]pyridin-7-yl)pyridine-3-sulfonamide (130.00 mg, 0.372 mmol, 1 equiv.) and (E)-4-ethoxy-1,1,1-trifluorobut-3-en-2-one (68.80 mg, 0.446 mmol, 1.2 equiv.) in MeCN (2 mL) was stirred for 2 h at 80° C. The mixture was concentrated under reduced pressure. The crude product mixture was used in the next step directly without further purification.

Step 3: Synthesis of N-(6-methoxy-1-methyl-1H-pyrazolo[4,3-c]pyridin-7-yl)-6-(5-(trifluoromethyl)-1H-pyrazol-1-yl)pyridine-3-sulfonamide (1-211). Into a 8 mL sealed tube were added 6-(5-hydroxy-5-(trifluoromethyl)-4,5-dihydro-1H-pyrazol-1-yl)-N-(6-methoxy-1-methyl-1H-pyrazolo[4,3-c]pyridin-7-yl)pyridine-3-sulfonamide (100.00 mg, 0.212 mmol, 1 equiv.), AcOH (2 mL) and DMF (2 mL) at room temperature. The reaction mixture was irradiated with microwave radiation for 2 h at 200° C. The resulting mixture was diluted with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃)+0.05% NH3. H₂O, mobile phase B: ACN; flow rate 60 mL/min; gradient: 16% B to 44% B in 7 min; wavelength: 254 nm/220 nm; Rt1(min): 8.23, to afford N-(6-methoxy-1-methyl-1H-pyrazolo[4,3-c]pyridin-7-yl)-6-(5-(trifluoromethyl)-1H-pyrazol-1-yl)pyridine-3-sulfonamide (7.2 mg, 7.30%) as an off-white solid. LCMS: (ES, m/z): [M+H]⁺ 454; (400 MHz, DMSO-d₆) δ 3.29 (s, 3H), 4.25 (s, 3H), 7.31 (d, J 2.0 Hz, 1H), 8.08 (d, J2.4 Hz, 1H), 8.10 (s, 1H), 8.23 (s, 1H), 8.26 (dd, J 8.4 Hz, 2.0 Hz, 1H), 8.65 (s, 1H), 8.71 (d, J 2.4 Hz, 1H), 10.26 (s, 1H).

### Example 151—Synthesis of 6-(4-Chloro-1H-Pyrazol-1-yl)-N-(6-Methoxy-1-(Methyl-D3)-1H-Indazol-7-yl)Pyridine-3-Sulfonamide (I-212)

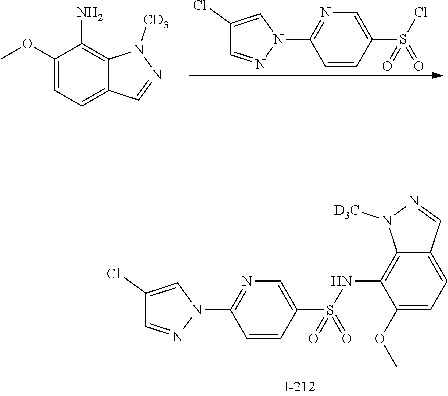 [SMILES: [2H]C([2H])([2H])n1ncc2ccc(OC)c(N)c21 ; O=S(=O)(Cl)c1ccc(-n2cc(Cl)cn2)nc1 ; [2H]C([2H])([2H])n1ncc2ccc(OC)c(NS(=O)(=O)c3ccc(-n4cc(Cl)cn4)nc3)c21]

A solution of 6-methoxy-1-(methyl-d3)-1H-indazol-7-amine (100 mg, 0.555 mmol, 1 equiv.) and 6-(4-chloropyrazol-1-yl)pyridine-3-sulfonyl chloride (169.74 mg, 0.611 mmol, 1.1 equiv.) in pyridine (6 mL) was stirred overnight at room temperature. The resulting mixture was concentrated under reduced pressure and diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm to afford 6-(4-chloro-1H-pyrazol-1-yl)-N-(6-methoxy-1-(methyl-d3)-1H-indazol-7-yl)pyridine-3-sulfonamide (76.8 mg, 32.19%) as a purple solid. LCMS: (ES, m/z): [M+H]⁺ 422; (400 MHz, DMSO-d₆) δ 3.18 (s, 3H), 6.82 (d, J8.8 Hz, 1H), 7.67 (t, J4.0 Hz, 1H), 7.99 (s, 1H), 8.08 (s, 1H), 8.10 (d, J8.4 Hz, 1H), 8.22(d, J8.4 Hz,1H), 8.62 (d, J2.0 Hz, 1H), 8.91 (s, 1H), 10.04 (s, 1H)

### Example 152—Synthesis of 6-(4-Chloro-1H-Pyrazol-1-yl)-N-(6-(Methoxy-D3)-1-Methyl-1H-Indazol-7-yl)Pyridine-3-Sulfonamide (I-213)

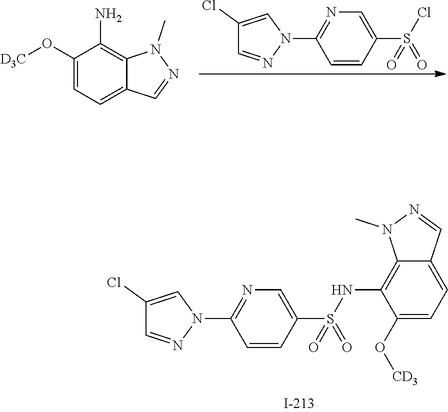 [SMILES: [2H]C([2H])([2H])Oc1ccc2cnn(C)c2c1N ; O=S(=O)(Cl)c1ccc(-n2cc(Cl)cn2)nc1 ; [2H]C([2H])([2H])Oc1ccc2cnn(C)c2c1NS(=O)(=O)c1ccc(-n2cc(Cl)cn2)nc1]

A solution of 6-(methoxy-d3)-1-methyl-1H-indazol-7-amine (100 mg, 0.555 mmol, 1 equiv.) and 6-(4-chloropyrazol-1-yl)pyridine-3-sulfonyl chloride (231.47 mg, 0.833 mmol, 1.5 equiv.) in pyridine (6 mL) was stirred overnight at room temperature under a nitrogen atmosphere. The resulting mixture was diluted with water (30 mL). The resulting mixture was extracted with EtOAc (3×10 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 0% to 100% gradient in 20 min; detector: UV 254 nm. This resulted in 6-(4-chloropyrazol-1-yl)-N-[6-(2H3) methoxy-1-methylindazol-7-yl]pyridine-3-sulfonamide (65.2 mg, 27.60%) as a purple solid. LCMS: (ES, m/z): [M+H]⁺ 422; ¹H NMR (400 MHz, DMSO-d₆) δ 4.27 (s, 3H), 6.82 (d, J8.8 Hz, 1H), 7.68 (d, J8.8 Hz, 1H), 7.99 (s, 1H), 8.08 (s, 1H), 8.10 (d, J8.4 Hz, 1H), 8.23 (dd, J8.8, 2.4 Hz, 1H), 8.63 (d, J2.4 Hz, 1H), 8.91 (s, 1H), 10.04 (s, 1H).

### Example 153—Synthesis of N-(1-Methyl-1H-Indazol-7-yl)-6-(2-(Trifluoromethyl)Thiazol-5-yl)Pyridine-3-Sulfonamide (I-214)

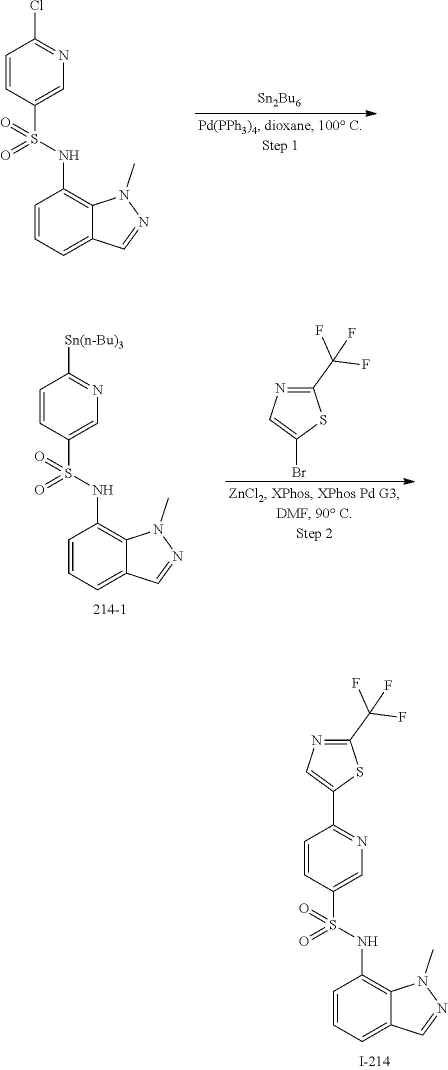

Step 1: Synthesis of 214-1. To a stirred solution of 6-chloro-N-(1-methylindazol-7-yl)pyridine-3-sulfonamide (200 mg, 0.620 mmol, 1 equiv.) and Pd(PPh₃)₄(71.6 mg, 0.062 mmol, 0.10 equiv.) in dioxane (3 mL) was added Sn₂(Bu)₆ (1.078 g, 1.858 mmol, 3.00 equiv.) in portions at room temperature under a nitrogen atmosphere. The resulting mixture was stirred for 15 h at 100° C. under a nitrogen atmosphere. The resulting mixture was concentrated under reduced pressure. The residue was purified by reversed-phase flash chromatography with the following conditions: column: C18 silica gel; mobile phase: MeCN in water (0.1% FA), 80% to 85% gradient in 40 min; detector: UV 215 nm. This resulted in 214-1 (70 mg, 19.57%) as a purple solid.

Step 2: Synthesis of N-(1-methyl-1H-indazol-7-yl)-6-(2-(trifluoromethyl)thiazol-5-yl)pyridine-3-sulfonamide (1-214). To a stirred mixture of 214-1 and 5-bromo-2-(trifluoromethyl)-1,3-thiazole (50 mg, 0.215 mmol, 1.78 equiv.) in DMF (4 mL) were added ZnCl₂ (50 mg, 0.367 mmol, 3.03 equiv.), XPhos (12 mg, 0.025 mmol, 0.21 equiv.) and XPhos Pd G3 (10 mg, 0.012 mmol, 0.10 equiv.) in portions at room temperature under a nitrogen atmosphere. The resulting mixture was stirred for 8 h at 90° C. The resulting mixture was concentrated under reduced pressure. The crude product (30 mg) was purified by prep-HPLC with the following conditions: column: Xselect CSH C18 OBD, 30*150 mm 5 m; mobile phase A: water(0.1% FA), mobile phase B: ACN; flow rate 60 mL/min; gradient: 47% B to 61% B in 7 min; wavelength: 254 nm/220 nm; Rt1(min): 6.7) to afford N-(1-methyl-1H-indazol-7-yl)-6-(2-(trifluoromethyl)thiazol-5-yl)pyridine-3-sulfonamide(9.2 mg, 17.03%) as a white solid. LCMS: (ES, m/z): [M+H]⁺ 440; (400 MHz, DMSO-d₆): δ 4.29 (s, 3H), 6.52 (d, J7.2 Hz, 1H), 6.93 (t, J7.6 Hz, 1H), 7.68 (d, J8.0 Hz, 1H), 8.09 (s, 1H), 8.20-8.22 (m, 1H), 8.40 (d, J8.0 Hz, 1H), 8.82 (s, 1H), 9.00 (s, 1H), 10.41 (s, 1H).

### Example 154—Synthesis of 6-(5-Cyclopropyl-1H-Pyrazol-1-yl)-N-(1-(2-Methoxyethyl)-1H-Indazol-7-yl)Pyridine-3-Sulfonamide (I-216)

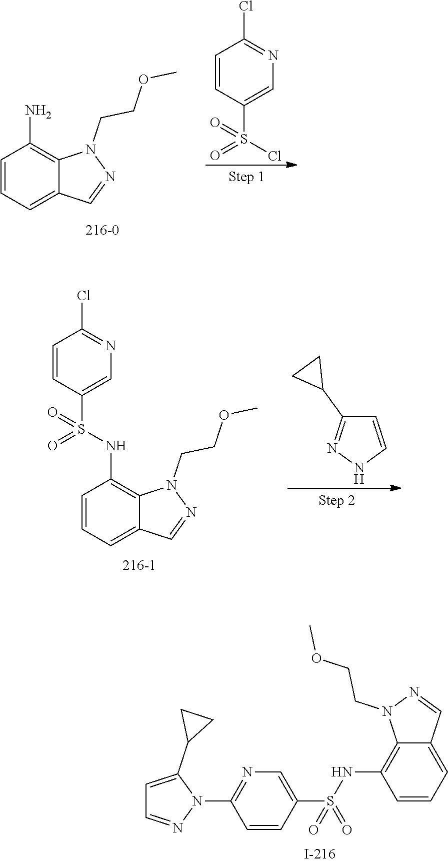

Step 1: Synthesis of 216-1. A solution of 1-(2-methoxyethyl)-1H-indazol-7-amine (800 mg, 4.183 mmol, 1.00 equiv.) and 6-chloropyridine-3-sulfonyl chloride (975.74 mg, 4.601 mmol, 1.10 equiv.) in pyridine (10 mL) was stirred at 80° C. under a nitrogen atmosphere. The mixture was allowed to cool down to room temperature. The resulting mixture was diluted with water (50 mL). The resulting mixture was extracted with EtOAc (3×50 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 70% to 50% gradient in 10 min; detector: UV 254 nm. This resulted in 6-chloro-N-(1-(2-methoxyethyl)-1H-indazol-7-yl)pyridine-3-sulfonamide (1.1 g, crude) as a purple solid.

Step 2: Synthesis of 6-(5-cyclopropyl-1H-pyrazol-1-yl)-N-(1-(2-methoxyethyl)-1H-indazol-7-yl)pyridine-3-sulfonamide (1-216). A solution of 6-chloro-N-(1-(2-methoxyethyl)-1H-indazol-7-yl)pyridine-3-sulfonamide (400 mg, 1.090 mmol, 1.00 equiv.), 3-cyclopropyl-1H-pyrazole (153.3 mg, 1.417 mmol, 1.30 equiv.) and Cs₂CO₃ (1.06 g, 3.270 mmol, 3.00 equiv.) in DMSO (10 mL) was stirred for 2 h at 140° C. under a nitrogen atmosphere. The mixture was allowed to cool down to room temperature. The resulting mixture was diluted with water (100 mL). The resulting mixture was extracted with EtOAc (3×100 mL). The combined organic layers were washed with brine (100 mL) and dried over anhydrous Na₂SO₄. After filtration, the filtrate was concentrated under reduced pressure. The crude product was purified by prep-HPLC with the following conditions: column: YMC-Actus Triant C18 ExRs, 30*150 mm, 5 m; mobile phase A: water (10 mmol/L NH₄HCO₃+0.05% NH₃H₂O), mobile phase B: ACN; flow rate 60 mL/min; gradient: 33% B to 60% B in 10 min; wavelength: 220 nm; Rt1(min): 9.52 to afford 6-(5-cyclopropyl-1H-pyrazol-1-yl)-N-(1-(2-methoxyethyl)-1H-indazol-7-yl)pyridine-3-sulfonamide (14.4 mg, 2.97%) as a light yellow solid. LCMS: (ES, m/z): [M+H]⁺ 439; (400 MHz, DMSO-d₆) δ 0.54-0.76 (m, 2H), 0.89-1.01 (m, 2H), 2.70-2.82 (m, 1H), 3.07 (s, 3H), 3.65 (t, J 5.2 Hz, 2H), 4.45 (t, J 5.2 Hz, 2H), 6.18 (d, J 2.0 Hz, 1H), 6.97 (t, J 7.6 Hz, 1H), 7.11 (d, J 7.2 Hz, 1H), 7.48 (d, J 8.4 Hz, 1H), 7.66 (d, J 1.6 Hz, 1H), 7.95 (d, J 8.8 Hz, 1H), 8.31 (s, 1H), 8.32 (d, J 2.4 Hz, 1H), 8.80 (d, J 2.4 Hz, 1H), 10.51 (s, 1H).

### Example 155—Synthesis of N-(1-(2-Methoxyethyl)-1H-Indazol-7-yl)-6-(4-Methyl-1H-Pyrazol-1-yl)Pyridine-3-Sulfonamide (I-217)

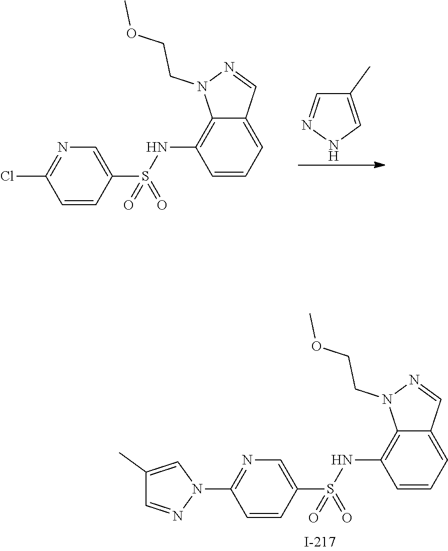

A solution of 6-chloro-N-(1-(2-methoxyethyl)-1H-indazol-7-yl)pyridine-3-sulfonamide (150 mg, 0.409 mmol, 1.00 equiv.), 4-methyl-1H-pyrazole (50.3 mg, 0.613 mmol, 1.50 equiv.), (1R,2R)—N1,N2-dimethylcyclohexane-1,2-diamine (46.5 mg, 0.327 mmol, 0.80 equiv.), CuI (38.9 mg, 0.204 mmol, 0.50 equiv.) and Cs₂CO₃ (399.7 mg, 1.227 mmol, 3.00 equiv.) in DMSO (5 mL) was stirred for 2 h at 140° C. under a nitrogen atmosphere. The mixture was allowed to cool down to room temperature. The resulting mixture was diluted with water (10 mL). The resulting mixture was extracted with EtOAc (3×15 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 50% to 60% gradient in 10 min; detector: UV 254 nm. This resulted in N-(1-(2-methoxyethyl)-1H-indazol-7-yl)-6-(4-methyl-1H-pyrazol-1-yl)pyridine-3-sulfonamide (48.5 mg, 28.55%) as a light yellow solid. LCMS: (ES, m/z): [M+H]⁺ 413; (400 MHz, DMSO-d₆) δ 2.09 (s, 3H), 3.12 (s, 3H), 3.68 (t, J 5.2 Hz, 2H), 4.46 (t, J 5.2 Hz, 2H), 6.83(d, J 8.8 Hz, 1H), 6.92 (t, J 5.2 Hz, 1H), 7.23 (d, J 2.4 Hz, 1H), 7.68 (s, 1H), 7.89 (d, J 8.8 Hz, 1H), 8.18 (s, 1H), 8.26 (d, J 7.2 Hz, 1H), 8.38 (s, 1H), 8.73 (s, 1H), 10.47 (s, 1H).

### Example 156—Synthesis of 6-(1,5-Dimethyl-1H-Pyrazol-4-yl)-N-(1-(2-Methoxyethyl)-1H-Indazol-7-yl)Pyridine-3-Sulfonamide (I-218)

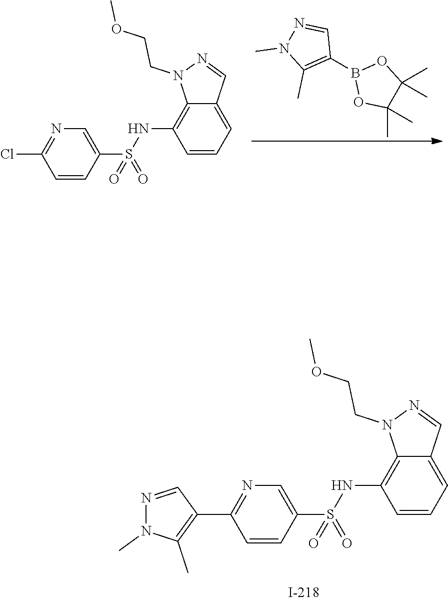

A solution of 6-chloro-N-(1-(2-methoxyethyl)-1H-indazol-7-yl)pyridine-3-sulfonamide (150 mg, 0.409 mmol, 1.00 equiv.), 1,5-dimethyl-4-(4,4,5,5-tetramethyl-1,3,2-dioxaborolan-2-yl)-1H-pyrazole (136.2 mg, 0.613 mmol, 1.50 equiv.), Pd(dppf)Cl₂CH₂Cl₂ (66.6 mg, 0.082 mmol, 0.20 equiv.) and Cs₂CO₃ (399.7 mg, 1.227 mmol, 3.00 equiv.) in dioxane (4 mL) and H₂O (1 mL) was stirred for 2 h at 80° C. under a nitrogen atmosphere. The mixture was allowed to cool down to room temperature. The resulting mixture was diluted with water (15 mL). The resulting mixture was extracted with EtOAc (3×15 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 40% to 50% gradient in 10 min; detector: UV 254 nm. This resulted in 6-(1,5-dimethyl-1H-pyrazol-4-yl)-N-(1-(2-methoxyethyl)-1H-indazol-7-yl)pyridine-3-sulfonamide (61.6 mg, 34.76%) as an off-white solid. LCMS: (ES, m/z): [M+H]⁺ 413; (400 MHz, DMSO-d₆) δ 2.57 (s, 3H), 3.17 (s, 3H), 3.75 (d, J 5.6 Hz, 5H), 4.47 (t, J 5.4 Hz, 2H), 6.77 (d, J 2.8 Hz, 1H), 6.85 (t, J 7.6 Hz, 1H), 7.00-7.20 (d, J 7.2 Hz, 1H), 7.63 (d, J 8.4 Hz, 1H), 7.89 (s, 1H), 8.05(d, J 8.4 Hz, 1H), 8.14 (s, 1H), 8.85 (d, J 2.4 Hz, 1H), 10.35 (s, 1H).

### Example 157—Synthesis of 6′-Chloro-N-(1-Methyl-1H-Indazol-7-yl)-[2,3′-Bipyridine]-5-Sulfonamide (I-219)

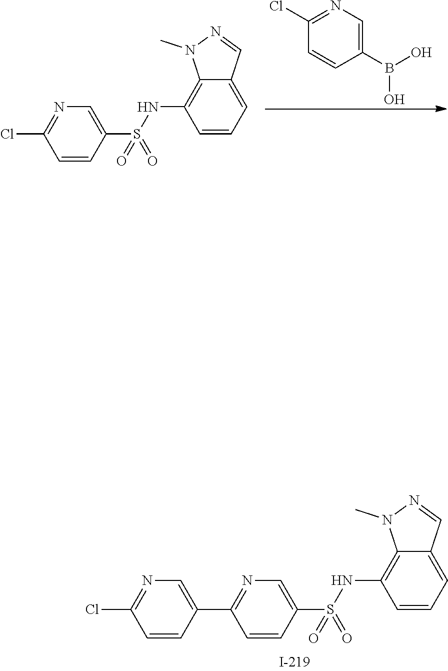 [SMILES: Cn1ncc2cccc(NS(=O)(=O)c3ccc(Cl)nc3)c21 ; OB(O)c1ccc(Cl)nc1 ; Cn1ncc2cccc(NS(=O)(=O)c3ccc(-c4ccc(Cl)nc4)nc3)c21]

A solution of 6-chloro-N-(1-methylindazol-7-yl)pyridine-3-sulfonamide (150 mg, 0.465 mmol, 1 equiv.), Pd(dppf)Cl₂CH₂Cl₂ (37.86 mg, 0.047 mmol, 0.1 equiv.), 6-chloropyridin-3-ylboronic acid (109.69 mg, 0.698 mmol, 1.5 equiv.) and Cs₂CO₃ (454.25 mg, 1.395 mmol, 3 equiv.) in dioxane (1.2 mL) and H₂O (0.3 mL) was stirred for 16 h at 110° C. under a nitrogen atmosphere. The mixture was allowed to cool down to room temperature. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 6′-chloro-N-(1-methylindazol-7-yl)-[2,3′-bipyridine]-5-sulfonamide (18.6 mg, 9.99%) as a white solid. LCMS: (ES, m/z): [M+H]⁺ 401; (400 MHz, DMSO-d₆) δ 4.30 (s, 3H), 6.54 (d, J7.2 Hz, 1H), 6.93 (t, J7.6 Hz, 1H), 7.67 (d, J8.0 Hz, 1H), 7.73 (d, J8.4 Hz, 1H), 8.09 (s, 1H), 8.17 (dd, J 8.4, 2.4 Hz, 1H), 8.34 (d, J 8.4 Hz, 1H), 8.60 (dd, J 8.4, 2.4 Hz, 1H), 8.90 (d, J2.4 Hz, 1H), 9.20 (d, J2.4 Hz, 1H), 10.39 (s, 1H).

### Example 158—Synthesis of 2′-Chloro-N-(1-Methyl-1H-Indazol-7-yl)-[2,3′-Bipyridine]-5-Sulfonamide (I-220)

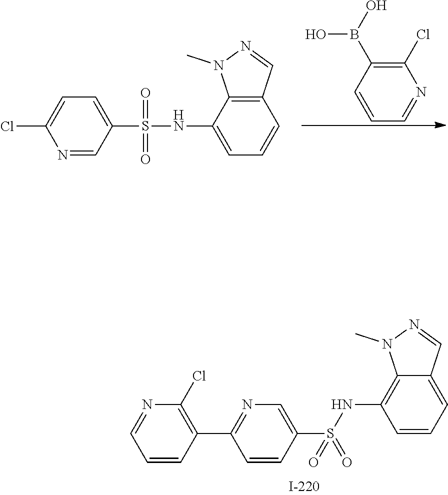 [SMILES: Cn1ncc2cccc(NS(=O)(=O)c3ccc(Cl)nc3)c21 ; OB(O)c1cccnc1Cl ; Cn1ncc2cccc(NS(=O)(=O)c3ccc(-c4cccnc4Cl)nc3)c21]

A solution of 6-chloro-N-(1-methylindazol-7-yl)pyridine-3-sulfonamide (100 mg, 0.310 mmol, 1 equiv.) in 1,4-dioxane (3 mL) was treated with 2-chloropyridin-3-yl boronic acid (73.13 mg, 0.465 mmol, 1.5 equiv.) for 2 min at room temperature under a nitrogen atmosphere followed by the addition of Pd(PPh₃)₄(71.60 mg, 0.062 mmol, 0.2 equiv.) and Cs₂CO₃ (302.83 mg, 0.930 mmol, 3 equiv.) in portions at 80° C. The resulting mixture was stirred for an additional 2 h at 80° C. The resulting mixture was concentrated under reduced pressure. The residue was purified by reversed-phase flash chromatography with the following conditions: column: C18 silica gel; mobile phase: MeCN in water (0.1% FA), 0% to 50% gradient in 20 min; detector: UV 254 nm. This resulted in 2′-chloro-N-(1-methyl-1H-indazol-7-yl)-[2,3′-bipyridine]-5-sulfonamide (31.7 mg, 25.56%) as a white solid. LCMS: (ES, m/z): [M+H]⁺400; (400 MHz, DMSO-d₆): δ 4.27 (s, 3H), 6.55 (d, J7.2 Hz, 1H), 6.94 (t, J7.6 Hz, 1H), 7.61-7.70 (m, 2H), 8.01 (d, J8.0 Hz, 1H), 8.09-8.20 (m, 3H), 8.56 (d, J2.8 Hz, 1H), 8.90 (s, 1H), 10.42 (s, 1H).

### Example 159—Synthesis of 2′-(Dimethylamino)-N-(1-Methyl-1H-Indazol-7-yl)-[2,3′-Bipyridine]-5-Sulfonamide (I-221)

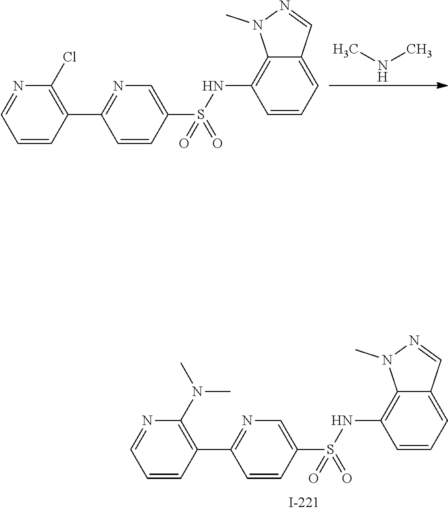

A solution of 2′-chloro-N-(1-methylindazol-7-yl)-[2,3′-bipyridine]-5-sulfonamide (100 mg, 0.250 mmol, 1 equiv.) in DMF (3 mL) was treated with dimethylamine (16.91 mg, 0.375 mmol, 1.5 equiv.) for 2 min at room temperature under a nitrogen atmosphere followed by the addition of Cs₂CO₃ (244.46 mg, 0.750 mmol, 3.00 equiv.) in portions at 60° C. The resulting mixture was stirred for an additional 2 h at 60° C. The resulting mixture was concentrated under reduced pressure. The residue was purified by reversed-phase flash chromatography with the following conditions: column: C18 silica gel; mobile phase: MeCN in water (0.1% FA), 0% to 50% gradient in 20 min; detector: UV 254 nm. This resulted in 2′-(dimethylamino)-N-(1-methyl-1H-indazol-7-yl)-[2,3′-bipyridine]-5-sulfonamide (60.6 mg, 59.14%) as a yellow solid. LCMS: (ES, m/z): [M+H]⁺409; (400 MHz, DMSO-d₆): δ 2.71 (s, 6H), 4.28 (s, 3H), 6.51 (d, J7.2 Hz, 1H), 6.88-6.95 (m, 2H), 7.67 (d, J8.0 Hz, 1H), 7.80-7.85 (m, 2H), 8.04 (dd, J8.4, 2.4 Hz, 1H), 8.09 (s, 1H), 8.24 (dd, J4.8, 2.0 Hz, 1H), 8.81 (d, J2.0 Hz, 1H), 10.31 (s, 1H).

### Example 160—Synthesis of N-(6-Methoxy-1-Methyl-1H-Indazol-7-yl)-6-(2-Oxooxazolidin-3-yl)Pyridine-3-Sulfonamide (I-222)

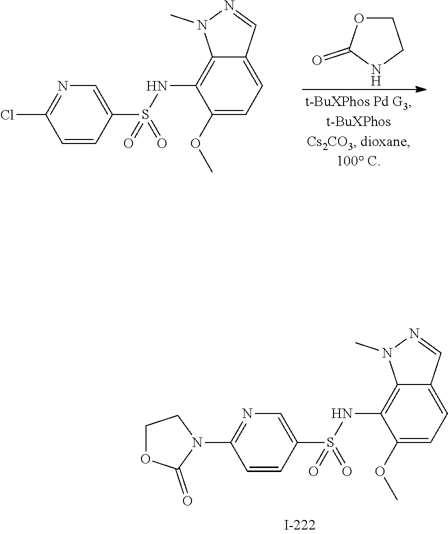 [SMILES: COc1ccc2cnn(C)c2c1NS(=O)(=O)c1ccc(Cl)nc1 ; O=C1NCCO1 ; COc1ccc2cnn(C)c2c1NS(=O)(=O)c1ccc(N2CCOC2=O)nc1]

To a stirred solution of 6-chloro-N-(6-methoxy-1-methylindazol-7-yl)pyridine-3-sulfonamide (405.14 mg, 1.148 mmol, 1 equiv.) and oxazolidinone (100 mg, 1.148 mmol, 1.00 equiv.) in dioxane (5 mL) were added t-BuXPhos (243.83 mg, 0.574 mmol, 0.5 equiv.), t-BuXPhos Pd G3 (456.71 mg, 0.574 mmol, 0.5 equiv.) and Cs₂CO₃ (1122.51 mg, 3.444 mmol, 3 equiv.) at room temperature under a nitrogen atmosphere. The resulting mixture was stirred overnight at 90° C. under a nitrogen atmosphere. The resulting mixture was diluted with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. This resulted in N-(6-methoxy-1-methylindazol-7-yl)-6-(2-oxo-1,3-oxazolidin-3-yl)pyridine-3-sulfonamide (61.9 mg, 13.21%) as a white solid. LCMS: (ES, m/z): [M+H]⁺ 404; ¹H NMR (400 MHz, DMSO-d₆) δ 3.34 (s, 3H), 4.21 (t, J 8.0 Hz, 2H), 4.26 (s, 3H), 4.50 (t, J 8.0 Hz, 2H), 6.83 (d, J 8.8 Hz, 1H), 7.67 (d, J 8.8 Hz, 1H), 7.98 (s, 1H), 8.05 (dd, J 9.0, 2.4 Hz, 1H), 8.25 (d, J 8.8 Hz, 1H), 8.51 (d, J 2.4 Hz, 1H), 9.89 (s, 1H).

### Example 161—Synthesis of 6-(5,5-Dimethyl-2-Oxooxazolidin-3-yl)-N-(6-Methoxy-1-Methyl-1H-Indazol-7-yl)Pyridine-3-Sulfonamide (I-223)

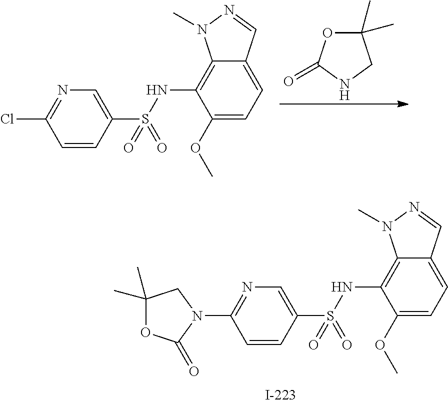

A solution of 6-chloro-N-(6-methoxy-1-methylindazol-7-yl)pyridine-3-sulfonamide (100 mg, 0.283 mmol, 1 equiv.), t-BuOK (95.42 mg, 0.849 mmol, 3 equiv.) and 5,5-dimethyl-1,3-oxazolidin-2-one (81.59 mg, 0.707 mmol, 2.5 equiv.) in DMSO (5 mL) was stirred overnight at 140° C. under a nitrogen atmosphere. The resulting mixture was diluted with water (10 mL) and extracted with EtOAc (3×10 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 0% to 100% gradient in 20 min; detector: UV 254 nm. This resulted in N-(6-methoxy-1-methylindazol-7-yl)-6-(5-methyl-2-oxo-1,3-oxazolidin-3-yl)pyridine-3-sulfonamide (18.1 mg, 15.25%) as a white solid. LCMS: (ES, m/z): [M+H]⁺ 432; ¹H NMR (400 MHz, DMSO-d₆) δ 1.50 (s, 6H), 3.21 (s, 3H), 4.01 (s, 2H), 4.26 (s, 3H), 6.82 (d, J8.8 Hz, 1H), 7.64 (d, J8.8 Hz, 1H), 7.96 (s, 1H), 8.05 (dd, J8.8, 2.4 Hz, 1H), 8.24 (d, J8.8 Hz, 1H), 8.49 (d, J2.4 Hz, 1H), 9.55 (s, 1H).

### Example 162-Synthesis of 6-(5,5-Dimethyl-2-Oxooxazolidin-3-yl)-N-(1-Methyl-1H-Indazol-7-yl)Pyridine-3-Sulfonamide (I-224)

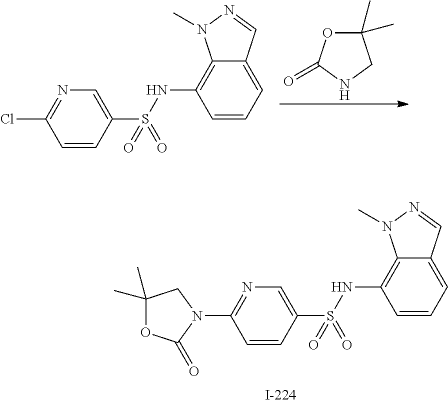

A solution of 6-chloro-N-(1-methylindazol-7-yl)pyridine-3-sulfonamide (200 mg, 0.620 mmol, 1 equiv.), trans-1,2-diaminocyclohexane (105 mg, 0.920 mmol, 1.48 equiv.), 5,5-dimethyl-1,3-oxazolidin-2-one (140 mg, 1.216 mmol, 1.96 equiv.), CuI (236 mg, 1.239 mmol, 2.00 equiv.) and Cs₂CO₃ (600 mg, 1.842 mmol, 2.97 equiv.) in DMSO (4 mL) was stirred for 2 h at 140° C. under a nitrogen atmosphere. The resulting mixture was diluted with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃)+0.05% NH3. H₂O, mobile phase B: ACN; flow rate 60 mL/min; gradient: 10% B to 37% B in 7 min; wavelength: 254 nm/220 nm; Rt1(min): 7.47) to afford 6-(5,5-dimethyl-2-oxo-1,3-oxazolidin-3-yl)-N-(1-methylindazol-7-yl)pyridine-3-sulfonamide (9.9 mg, 3.97%) as an off-white solid. LCMS: (ES, m/z): [M+H]⁺ 401; ¹H NMR (400 MHz, DMSO-d₆): δ 1.46 (s, 6H), 3.95 (s, 2H), 4.36 (s, 3H), 6.70 (d, J 2.8 Hz, 1H), 6.73 (t, J 7.6 Hz, 1H), 7.04 (s, 1H), 7.80 (s, 1H), 8.08 (d, J 2.0 Hz, 1H),8.09(d, J 2.0 Hz, 1H), 8.58 (s, 1H).

### Example 163—Synthesis of N-(6-Methoxy-1-Methyl-1H-Indazol-7-yl)-6-(3-Methyl-2-Oxoimidazolidin-1-yl)Pyridine-3-Sulfonamide (I-225)

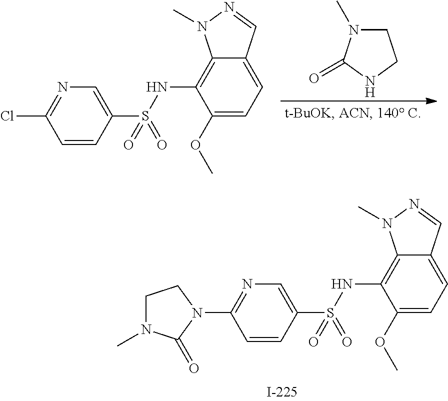

A mixture of 6-chloro-N-(6-methoxy-1-methylindazol-7-yl)pyridine-3-sulfonamide (150 mg, 0.425 mmol, 1 equiv.), t-BuOK (143.13 mg, 1.275 mmol, 3 equiv.) and 1-methylimidazolidin-2-one (85.14 mg, 0.850 mmol, 2 equiv.) in DMSO (3 mL) was stirred overnight at 140° C. The resulting mixture was diluted with water (30 mL). The resulting mixture was extracted with EA (3×30 mL). The combined organic layers were washed with brine (100 mL) and dried over anhydrous Na₂SO₄. After filtration, the filtrate was concentrated under reduced pressure. The crude product (110 mg) was purified by prep-HPLC with the following conditions: column: XBridge Prep OBD C18, 30*150 mm, 5 m; mobile phase A: water(10 mmol/L NH₄HCO₃+0.05% NH₃H₂O), mobile phase B: ACN; flow rate 60 mL/min; gradient: 20% B to 31% B in 9 min; wavelength: 220 nm; Rt1(min): 8.11) to afford N-(6-methoxy-1-methylindazol-7-yl)-6-(3-methyl-2-oxoimidazolidin-1-yl)pyridine-3-sulfonamide (29.9 mg, 16.84%) as a white solid. LCMS: (ES, m/z): [M+H]⁺ 417; (400 MHz, DMSO-d₆) δ 2.82 (s, 3H), 3.19 (s, 3H), 3.49 (t, J 8.0 Hz, 2H), 3.98 (t, J 8.8 Hz, 2H), 4.25 (s, 3H), 6.82 (d, J 8.8 Hz, 1H), 7.65 (d, J 8.8 Hz, 1H), 7.90 (dd, J 9.2, 2.4 Hz, 1H), 7.97 (s, 1H), 8.34 (d, J 9.2 Hz, 1H), 8.40 (d, J 2.4 Hz, 1H), 9.75 (s, 1H).

### Example 164—Synthesis of N-(1-Methyl-1H-Indazol-7-yl)-6-(3-Methyl-2-Oxoimidazolidin-1-yl)Pyridine-3-Sulfonamide (I-226)

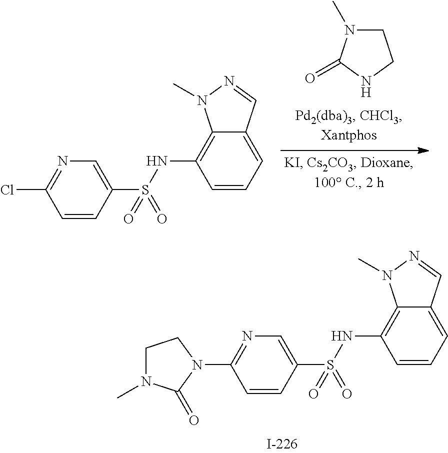

A solution of 6-chloro-N-(1-methylindazol-7-yl)pyridine-3-sulfonamide (200 mg, 0.620 mmol, 1 equiv.), 1-methylimidazolidin-2-one (120 mg, 1.199 mmol, 1.93 equiv.), XantPhos (70 mg, 0.121 mmol, 0.20 equiv.), KI (200 mg, 1.205 mmol, 1.94 equiv.), Cs₂CO₃ (600 mg, 1.842 mmol, 2.97 equiv.) and Pd₂(dba)₃CHCl₃ (120 mg, 0.116 mmol, 0.19 equiv.) in 1,4-dioxane (5 mL) was stirred for 2 h at 100° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃+0.05% NH3. H₂O), mobile phase B: ACN; flow rate 60 mL/min; gradient: 10% B to 33% B in 7 min; wavelength: 254 nm/220 nm; Rt1(min): 7.3) to afford N-(1-methyl-1H-indazol-7-yl)-6-(3-methyl-2-oxoimidazolidin-1-yl)pyridine-3-sulfonamide (48.5 mg, 20.01%) as an off-white solid. LCMS: (ES, m/z): [M+H]⁺ 387; (400 MHz, DMSO-d₆): δ2.83 (s, 3H), 3.49 (dd, J 8.8, 7.2 Hz, 2H), 3.97 (dd, J 9.2, 7.2 Hz, 2H), 4.27 (s, 3H), 6.48 (dd, J 7.2, 1.2 Hz, 1H), 6.93 (t, J 7.6 Hz, 1H), 7.68 (d, J 8.0 Hz, 1H), 7.94 (dd, J 9.2, 2.4 Hz, 1H), 8.08 (s, 1H), 8.38 (d, J 8.8 Hz, 1H), 8.41 (d, J 2.0 Hz, 1H), 10.05 (s, 1H).

### Example 165—Synthesis of N-(1-Methyl-1H-Indazol-7-yl)-6-(4,5,6,7-Tetrahydro-1H-Indazol-1-yl)Pyridine-3-Sulfonamide (I-227)

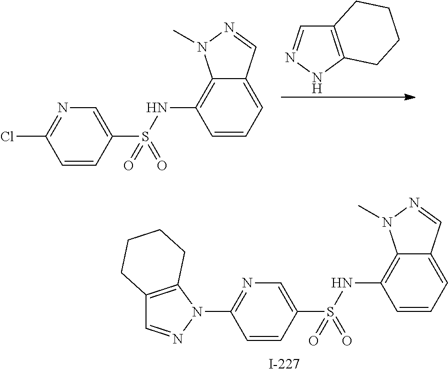

To a stirred solution of 6-chloro-N-(1-methylindazol-7-yl)pyridine-3-sulfonamide (200 mg, 0.620 mmol, 1 equiv.) and 4,5,6,7-tetrahydro-1H-indazole (113.55 mg, 0.930 mmol, 1.5 equiv.) in DMSO (100 mL) were added Cs₂CO₃ (605.67 mg, 1.860 mmol, 3 equiv.) in portions at 140° C. under an air atmosphere. The resulting mixture was stirred for an additional 2 h at 140° C. The crude product (200 mg) was purified by prep-HPLC with the following conditions: column: Xselect CSH C18 OBD, 30*150 mm 5 m; mobile phase A: water (0.1% FA), mobile phase B: ACN; flow rate 60 mL/min; gradient: 47% B to 57% B in 7 min; wavelength: 254 nm/220 nm; Rt1(min): 8.75, 9.5(min) to afford N-(1-methylindazol-7-yl)-6-(4,5,6,7-tetrahydroindazol-1-yl)pyridine-3-sulfonamide (21.9 mg, 8.42%) as a white solid. LCMS: (ES, m/z): [M+H]⁺ 402; ¹H NMR (400 MHz, DMSO-d₆) 1.69-1.78 (m, 4H), 2.58 (s, 2H), 3.12 (s, 2H), 4.29 (s, 3H), 6.52 (d, J 7.2 Hz, 1H), 6.94 (t, J7.6 Hz, 1H), 7.66-7.70 (m, 2H), 8.01-8.16 (m, 3H), 8.56 (s, 1H), 10.27 (s, 1H).

### Example 166—Synthesis of 4-Bromo-1-Methyl-2-(Trifluoromethyl) Imidazole

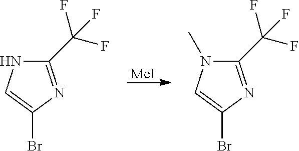

A mixture of 4-bromo-2-(trifluoromethyl)-1H-imidazole (500 mg, 2.326 mmol, 1 equiv.), Cs₂CO₃ (2273.44 mg, 6.978 mmol, 3 equiv.) and methyl iodide (990.40 mg, 6.978 mmol, 3 equiv.) in THE (6 mL) was stirred for 3 hours at room temperature. The resulting mixture was diluted with water (50 mL). The resulting mixture was extracted with CH₂Cl₂ (3×50 mL). The combined organic layers were washed with brine (100 mL) and dried over anhydrous Na₂SO₄. After filtration, the filtrate was concentrated under reduced pressure. This resulted in 4-bromo-1-methyl-2-(trifluoromethyl)imidazole (300 mg, crude) as a yellow oil.

### Example 167—Synthesis of N-(1-Methyl-1H-Indazol-7-yl)-6-(1-Methyl-2-(Trifluoromethyl)-1H-Imidazol-4-yl)Pyridine-3-Sulfonamide (I-228)

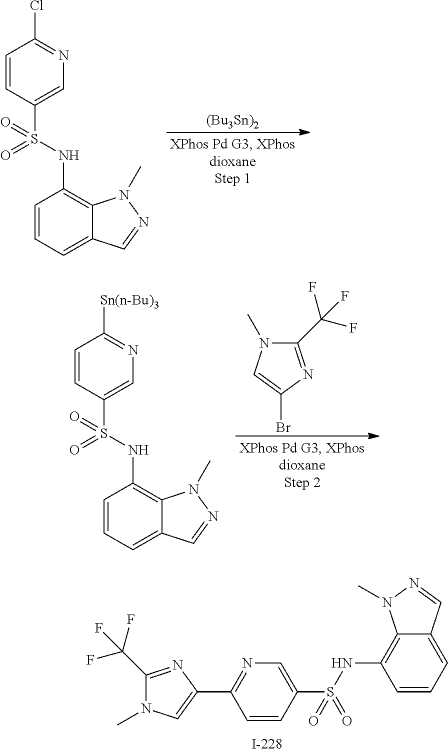

Step 1: Synthesis of N-(1-methylindazol-7-yl)-6-(tributylstannyl)pyridine-3-sulfonamide. A mixture of 6-chloro-N-(1-methylindazol-7-yl)pyridine-3-sulfonamide (2000 mg, 6.20 mmol, 1 equiv.), hexabutyldistannane (7189 mg, 12.4 mmol, 2 equiv.) and XPhos Pd G3 (1049 mg, 1.20 mmol, 0.2 equiv.), XPhos (591 mg, 1.20 mmol, 0.2 equiv.) in dioxane (10 mL) was stirred overnight at 110° C. under a nitrogen atmosphere. The resulting mixture was diluted with water (50 mL). The resulting mixture was extracted with CH₂Cl₂ (3×50 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 10% to 50% gradient in 10 min; detector: UV 254 nm. This resulted in N-(1-methylindazol-7-yl)-6-(tributylstannyl)pyridine-3-sulfonamide (230 mg, 6.41%) as a red solid.

Step 2: Synthesis of N-(1-methyl-1H-indazol-7-yl)-6-(1-methyl-2-(trifluoromethyl)-1H-imidazol-4-yl)pyridine-3-sulfonamide (1-228). To a stirred solution of N-(1-methylindazol-7-yl)-6-(tributylstannyl)pyridine-3-sulfonamide (230 mg, 0.398 mmol, 1 equiv.) and 4-bromo-1-methyl-2-(trifluoromethyl)imidazole (182.44 mg, 0.796 mmol, 2 equiv.) in dioxane (2 mL) was added XPhos Pd G3 (67.44 mg, 0.080 mmol, 0.2 equiv.) and XPhos (37.98 mg, 0.080 mmol, 0.2 equiv.) under a nitrogen atmosphere. The resulting mixture was stirred at 110° C. under a nitrogen atmosphere overnight. The resulting mixture was diluted with water (50 mL). The resulting mixture was extracted with CH₂Cl₂ (3×50 mL). The combined organic layers were washed with brine (100 mL) and dried over anhydrous Na₂SO₄. After filtration, the filtrate was concentrated under reduced pressure. The crude product (120 mg) was purified by prep-HPLC with the following conditions: column: YMC-Actus Triant C18 ExRs, 30*150 mm, 5 m; mobile phase A: water (10 mmol/L NH₄HCO₃+0.05% NH₃H₂O), mobile phase B: ACN; flow rate 60 mL/min; gradient: 5% B to 37% B in 8 min; wavelength: 220 nm; Rt1(min): 7.58) to afford 6-[1-methyl-2-(trifluoromethyl)imidazol-4-yl]-N-(1-methylindazol-7-yl)pyridine-3-sulfonamide (24.2 mg, 13.56%) as an off-white solid. LCMS: (ES, m/z): [M+H]⁺ 437; (400 MHz, DMSO-d₆) δ 3.90 (s, 3H), 4.27 (s, 3H), 6.49 (d, J 7.2 Hz, 1H), 6.92 (t, J 7.8 Hz, 1H), 7.68 (d, J 8.0 Hz, 1H), 8.06 (d, J=8.4 Hz, 1H), 8.07 (s, 1H), 8.08 (dd, J=4.4, 1.6 Hz, 1H), 8.27 (s, 1H), 8.68 (s, 1H), 10.25 (s, 1H).

### Example 168—Synthesis of 6-(4-Cyclopropyl-1H-Pyrazol-1-yl)-N-(6-Methoxy-1-(Methyl-D3)-1H-Indazol-7-yl)Pyridine-3-Sulfonamide (I-229)

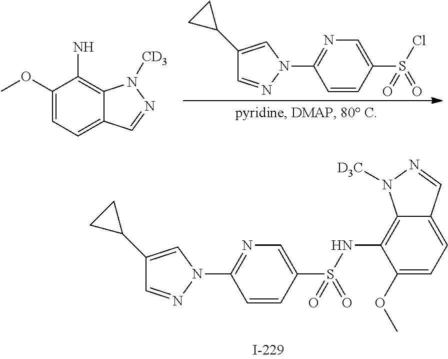 [SMILES: [2H]C([2H])([2H])n1ncc2ccc(OC)c(N)c21 ; O=S(=O)(Cl)c1ccc(-n2cc(C3CC3)cn2)nc1 ; [2H]C([2H])([2H])n1ncc2ccc(OC)c(NS(=O)(=O)c3ccc(-n4cc(C5CC5)cn4)nc3)c21]

A solution of 6-methoxy-1-(2H3)methylindazol-7-amine (100 mg, 0.555 mmol, 1 equiv.), DMAP (20.34 mg, 0.167 mmol, 0.3 equiv.) and 6-(4-cyclopropylpyrazol-1-yl)pyridine-3-sulfonyl chloride (236.15 mg, 0.833 mmol, 1.5 equiv.) in pyridine (2 mL) was stirred for 2 h at 80° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by prep-HPLC with the following conditions: column: XBridge Prep OBD C18, 30*150 mm, 5 m; mobile phase A: water (10 mmol/L NH₄HCO₃+0.05% NH₃·H₂O), mobile phase B: ACN; flow rate 60 mL/min; gradient: 40% B to 70% B in 7 min; wavelength: 254 nm/220 nm; Rt1(min): 6) to afford 6-(4-cyclopropylpyrazol-1-yl)-N-[6-methoxy-1-(2H3)methylindazol-7-yl]pyridine-3-sulfonamide (29.5 mg, 12.01%) as a white solid. LCMS: (ES, m/z): [M+H]⁺ 428; ¹H NMR (400 MHz, DMSO-d₆) δ 0.66-0.68 (m, 2H), 0.89-0.92 (m, 2H), 1.78-1.82 (m, 1H), 3.18 (s, 3H), 6.82 (d, J 8.8 Hz, 1H), 7.66 (d, J 8.8 Hz, 1H), 7.74 (d, J 0.8 Hz, 1H), 7.98 (s, 1H), 8.02 (d, J 8.4 Hz, 1H), 8.14 (dd, J 8.8, 2.4 Hz, 1H), 8.44 (s, 1H), 8.56 (d, J 2.4 Hz, 1H), 10.01 (s, 1H).

### Example 169—Synthesis of N-(1-Methyl-1H-Indazol-7-yl)-6-(2-Oxooxazolidin-3-yl)Pyridine-3-Sulfonamide (I-230)

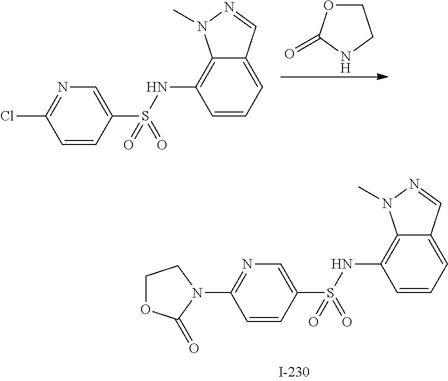

A mixture of 6-chloro-N-(1-methyl-1H-indazol-7-yl)pyridine-3-sulfonamide (200.00 mg, 0.620 mmol, 1 equiv.), oxazolidin-2-one (64.75 mg, 0.744 mmol, 1.2 equiv.), Pd₂(dba)₃CHCl₃ (128.28 mg, 0.124 mmol, 0.2 equiv.), XantPhos (71.71 mg, 0.124 mmol, 0.2 equiv.) and Cs₂CO₃ (605.67 mg, 1.860 mmol, 3 equiv.) in dioxane (3 mL) was stirred for 16 h at 100° C. under a nitrogen atmosphere. The resulting mixture was diluted with water (15 mL). The resulting mixture was extracted with EtOAc (3×15 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 10% to 50% gradient in 10 min; detector: UV 254 nm, to afford N-(1-methyl-1H-indazol-7-yl)-6-(2-oxooxazolidin-3-yl)pyridine-3-sulfonamide (60.30 mg, 25.98%) as an off-white solid. LCMS: (ES, m/z): [M+H]⁺ 374; (400 MHz, DMSO-d₆) δ 4.20 (t, J 8.2 Hz, 2H), 4.29 (s, 3H), 4.48 (t, J 8.2 Hz, 2H), 6.53 (d, J 7.2 Hz, 1H), 6.89 (t, J 7.6 Hz, 1H), 7.56 (s, 1H), 8.02 (s, 1H), 8.09 (d, J 8.4 Hz, 1), 8.26 (d, J 9.2 Hz, 1H), 8.54 (s, 1H), 10.15 (s, 1H).

### Example 170—Synthesis of 6-(3-Cyclopropyl-1H-Pyrazol-1-yl)-N-(1-(2-Methoxyethyl)-1H-Indazol-7-yl)Pyridine-3-Sulfonamide (I-231)

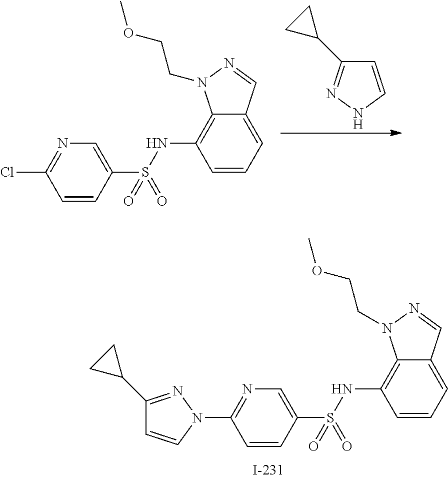 [SMILES: COCCn1ncc2cccc(NS(=O)(=O)c3ccc(Cl)nc3)c21 ; c1cc(C2CC2)n[nH]1 ; COCCn1ncc2cccc(NS(=O)(=O)c3ccc(-n4ccc(C5CC5)n4)nc3)c21]

A solution of 6-chloro-N-(1-(2-methoxyethyl)-1H-indazol-7-yl)pyridine-3-sulfonamide (400 mg, 1.090 mmol, 1.00 equiv.), 3-cyclopropyl-1H-pyrazole (153.3 mg, 1.417 mmol, 1.30 equiv.) and Cs₂CO₃ (1.06 g, 3.270 mmol, 3.00 equiv.) in DMSO (5 mL) was stirred for 2 h at 140° C. under a nitrogen atmosphere. The mixture was allowed to cool down to room temperature. The resulting mixture was diluted with water (20 mL). The resulting mixture was extracted with EtOAc (3×20 mL). The combined organic layers were washed with brine (60 mL) and dried over anhydrous Na₂SO₄. After filtration, the filtrate was concentrated under reduced pressure. The crude product was purified by prep-HPLC with the following conditions: Column: YMC-Actus Triant C18 ExRs, 30*150 mm, 5 μm; mobile phase A: water (10 mmol/L NH₄HCO₃+0.05% NH₃H₂O), mobile phase B: ACN; flow rate 60 mL/min; gradient: 33% B to 60% B in 10 min; wavelength: 220 nm; Rt1(min): 9.52 to afford to afford6-(3-cyclopropyl-1H-pyrazol-1-yl)-N-(1-(2-methoxyethyl)-1H-indazol-7-yl)pyridine-3-sulfonamide (60.2 mg, 12.55%) as an off-white solid. LCMS: (ES, m/z): [M+H]⁺ 439; (400 MHz, DMSO-d₆) δ 0.69-0.82 (m, 2H), 0.90-1.02 (m, 2H), 1.85-2.11 (m, 1H), 3.06 (s, 3H), 3.62 (t, J 5.6 Hz, 2H), 4.45 (t, J 5.2 Hz, 2H), 6.34 (d, J 2.4 Hz, 1H), 6.97 (t, J 7.6 Hz, 1H), 7.11 (d, J 7.2 Hz, 1H), 7.48 (d, J 8.4 Hz, 1H), 7.88 (d, J 8.4 Hz, 1H), 8.27 (d, J 2.4 Hz, 1H), 8.30 (s, 1H), 8.46 (d, J 2.4 Hz, 1H), 8.72 (d, J 2.4 Hz, 1H), 10.49 (s, 1H).

### Example 171—Synthesis of 6-(5-Isopropyl-1H-Pyrazol-1-yl)-N-(1-(2-Methoxyethyl)-1H-Indazol-7-yl)Pyridine-3-Sulfonamide (I-232)

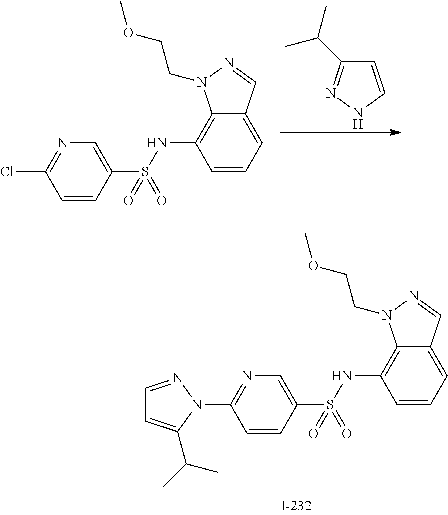

A mixture of 6-chloro-N-(1-(2-methoxyethyl)-1H-indazol-7-yl)pyridine-3-sulfonamide (200.00 mg, 0.545 mmol, 1 equiv.), 3-isopropyl-1H-pyrazole (72.07 mg, 0.654 mmol, 1.2 equiv.) and Cs₂CO₃ (532.94 mg, 1.635 mmol, 3 equiv.) in DMSO (2 mL) was stirred for 2 h at 140° C. The resulting mixture was diluted with water (15 mL) and extracted with EtOAc (3×15 mL). The combined organic layers were washed with brine (50 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃+0.05% NH₃. H₂O), mobile phase B: ACN; flow rate 60 mL/min; gradient: 38% B to 65% B in 7 min; wavelength: 254 nm/220 nm; Rt1(min): 8.3, to afford 6-(5-isopropyl-1H-pyrazol-1-yl)-N-(1-(2-methoxyethyl)-1H-indazol-7-yl)pyridine-3-sulfonamide (4.10 mg, 1.69%) as an off-white solid. LCMS: (ES, m/z): [M+H]⁺ 441; (400 MHz, DMSO-d₆) δ 1.18 (d, J 6.8 Hz, 6H), 3.09 (s, 3H), 3.66 (t, J 5.2 Hz, 2H), 3.86-4.00 (m, 1H), 4.44 (t, J 5.2 Hz, 2H), 6.40 (d, J 1.6 Hz, 1H), 6.95 (t, J 7.6 Hz, 1H), 7.08 (d, J 7.2 Hz, 1H), 7.44 (d, J 8.4 Hz, 1H), 7.68 (d, J 1.6 Hz, 1H), 7.94 (d, J 8.8 Hz, 1H), 8.28 (s, 1H), 8.30 (dd, J 8.4, 2.4 Hz, 1H), 8.78 (d, J 2.4 Hz, 1H), 10.32 (s, 1H).

### Example 172—Synthesis of N-(1-Methyl-1H-Indazol-7-yl)-6-(4,5,6,7-Tetrahydro-2H-Indazol-2-yl)Pyridine-3-Sulfonamide (I-233)

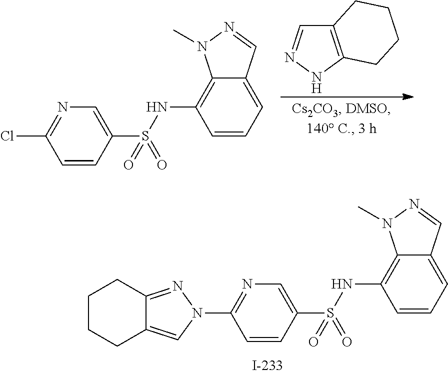

To a stirred solution of 6-chloro-N-(1-methylindazol-7-yl) pyridine-3-sulfonamide (100 mg, 0.310 mmol, 1 equiv.) and 4,5,6,7-tetrahydro-1H-indazole (45.42 mg, 0.372 mmol, 1.2 equiv.) in DMSO (5 mL) were added Cs₂CO₃ (302.83 mg, 0.930 mmol, 3 equiv.) in portions at 140° C. under a nitrogen atmosphere. The resulting mixture was stirred for an additional 3 h at 140° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃+0.05% NH₃. H₂O), mobile phase B: ACN; flow rate 60 mL/min; gradient: 10% B to 33% B in 7 min; wavelength: 254 nm/220 nm; Rt1(min): 7.3) to afford N-(1-methyl-1H-indazol-7-yl)-6-(4,5,6,7-tetrahydro-2H-indazol-2-yl)pyridine-3-sulfonamide (71.5 mg, 55.71%) as a white solid. LCMS: (ES, m/z): [M+H]⁺ 409; (400 MHz, DMSO-d₆): δ1.66-1.76 (m, 2H), 1.77-1.89 (m, 2H), 2.59 (t, J 6.4 Hz, 2H), 2.69 (t, J 6.4 Hz, 2H), 4.28 (s, 3H), 6.52 (d, J 7.2 Hz, 1H), 6.93 (t, J 7.8 Hz, 1H), 7.70 (d, J 8.0 Hz, 1H), 8.00 (d, J 8.8 Hz, 1H), 8.09 (s, 1H), 8.13 (dd, J 8.8, 2.4 Hz, 1H), 8.35 (s, 1H), 8.53 (d, J 2.0 Hz, 1H), 10.20 (s, 1H)

### Example 173—Synthesis of N-(1-Methyl-1H-Indazol-7-yl)-6-(1-Methyl-2-(Trifluoromethyl)-1H-Imidazol-5-yl)Pyridine-3-Sulfonamide (I-234)

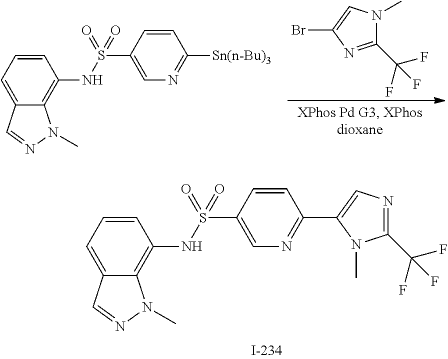

To a stirred solution of N-(1-methylindazol-7-yl)-6-(tributylstannyl)pyridine-3-sulfonamide (230 mg, 0.398 mmol, 1 equiv.) and 4-bromo-1-methyl-2-(trifluoromethyl)imidazole (182.44 mg, 0.796 mmol, 2 equiv.) in 1,4-dioxane (2 mL) was added XPhos Pd G3 (67.44 mg, 0.080 mmol, 0.2 equiv.) and XPhos (37.98 mg, 0.080 mmol, 0.2 equiv.) under a nitrogen atmosphere. The resulting mixture was stirred at 110° C. under a nitrogen atmosphere overnight. The resulting mixture was diluted with water (50 mL). The resulting mixture was extracted with CH₂Cl₂ (3×50 mL). The combined organic layers were washed with brine (100 mL) and dried over anhydrous Na₂SO₄. After filtration, the filtrate was concentrated under reduced pressure. The crude product (120 mg) was purified by prep-HPLC with the following conditions: column: YMC-Actus Triant C18 ExRs, 30*150 mm, 5 m; mobile phase A: water (10 mmol/L NH₄HCO₃+0.05% NH₃H₂O), mobile phase B: ACN; flow rate 60 mL/min; gradient: 5% B to 37% B in 8 min; wavelength: 220 nm; Rt1(min): 7.58) to afford 6-[3-methyl-2-(trifluoromethyl)imidazol-4-yl]-N-(1-methylindazol-7-yl)pyridine-3-sulfonamide (11.5 mg, 6.60%) as a white solid. LCMS: (ES, m/z): [M+H]⁺ 437; (400 MHz, DMSO-d₆) δ 4.10 (s, 3H), 4.29 (s, 3H), 6.54 (d, J 7.2 Hz, 1H), 6.92 (t, J 7.6 Hz, 1H), 7.66 (d, J 8.0 Hz, 1H), 7.87 (s, 1H), 8.08 (s, 1H), 8.13 (d, J 9.2 Hz, 1H), 8.15 (dd, J 8.4, 2.4 Hz, 1H), 8.83 (d, J 2.4 Hz, 1H), 10.37 (s, 1H).

### Example 174—Synthesis of 6-(4-(1-Fluorocyclopropyl)-1H-Pyrazol-1-yl)-N-(1-Methyl-1H-Indazol-7-yl)Pyridine-3-Sulfonamide (I-254)

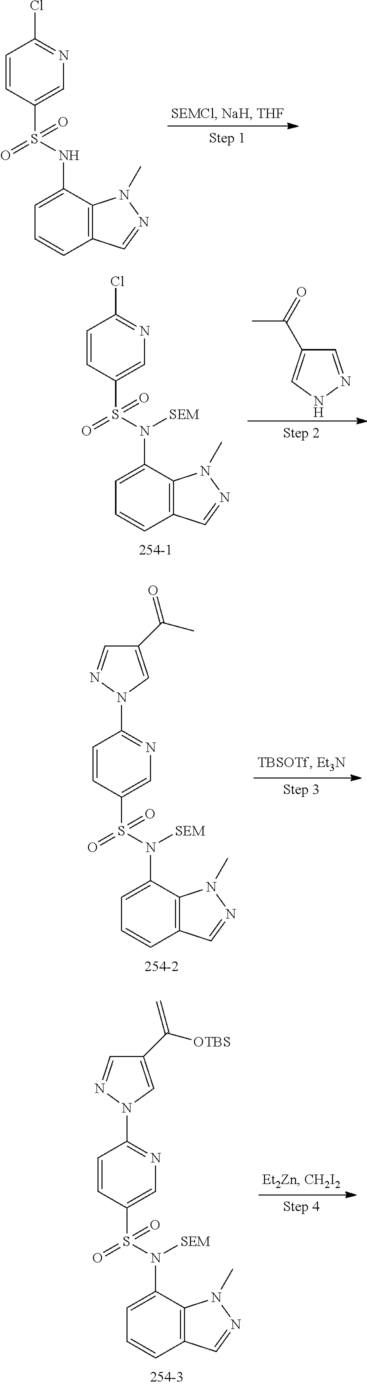

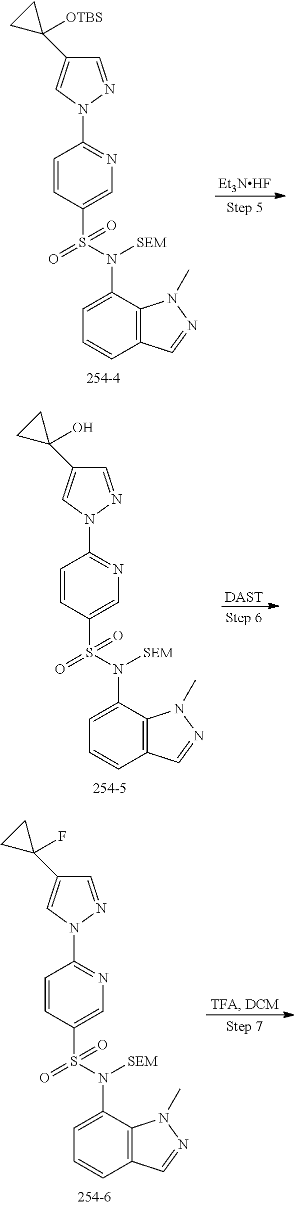

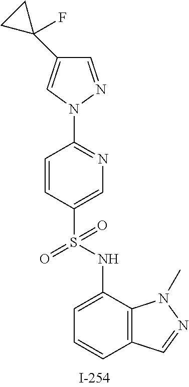 [SMILES: Cn1ncc2cccc(NS(=O)(=O)c3ccc(-n4cc(C5(F)CC5)cn4)nc3)c21]

Step 1: Synthesis of 254-1. To a stirred solution of 6-chloro-N-(1-methylindazol-7-yl)pyridine-3-sulfonamide (1 g, 3.098 mmol, 1 equiv.) in THE (20 mL) were added NaH (0.15 g, 6.196 mmol, 2.0 equiv.) in portions at 0° C. under a nitrogen atmosphere. The resulting mixture was stirred for an additional 30 min at 0° C. To the above mixture was added SEM-Cl (1.55 g, 9.294 mmol, 3.0 equiv.) dropwise over 10 min at 0° C. The resulting mixture was stirred for an additional 5 h at room temperature. The reaction was quenched by the addition of sat. NH₄Cl (aq.) (20 mL) and water/ice (20 mL) at 0° C. The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 40% to 70% gradient in 10 min; detector: UV 220 nm. To afford 6-chloro-N-(1-methylindazol-7-yl)-N-{[2-(trimethylsilyl)ethoxy]methyl}pyridine-3-sulfonamide (1.2 g, 85.50%) as a light yellow solid.

Step 2: Synthesis of 254-2. To a stirred mixture of 6-chloro-N-(1-methylindazol-7-yl)-N-{[2-(trimethylsilyl)ethoxy]methyl}pyridine-3-sulfonamide (800 mg, 1.766 mmol, 1 equiv.) and 1-(1H-pyrazol-4-yl)ethanone (291.68 mg, 2.649 mmol, 1.5 equiv.) in DMF (15 ml) were added Cs₂CO₃ (1726.08 mg, 5.298 mmol, 3.0 equiv.) at room temperature under a nitrogen atmosphere. The resulting mixture was stirred for an additional 6 h at 90° C. The reaction was quenched by the addition of water/ice (20 mL) at 0° C. The resulting mixture was extracted with CH₂Cl₂ (3×20 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₄HCO₃), 50% to 80% gradient in 10 min; detector: UV 220 nm. To afford 6-(4-acetylpyrazol-1-yl)-N-(1-methylindazol-7-yl)-N-{[2-(trimethylsilyl)ethoxy]methyl}pyridine-3-sulfonamide (600 mg, 64.51%) as a light brown solid.

Step 3: Synthesis of 254-3. To a stirred mixture of 6-(4-acetylpyrazol-1-yl)-N-(1-methylindazol-7-yl)-N-{[2-(trimethylsilyl)ethoxy]methyl}pyridine-3-sulfonamide (556 mg, 1.056 mmol, 1 equiv.) and Et₃N (320.47 mg, 3.168 mmol, 3 equiv.) in DCM (10 mL) were added TBSOTf (418.56 mg, 1.584 mmol, 1.5 equiv.) dropwise at 0° C. under a nitrogen atmosphere. The resulting mixture was stirred for an additional 5 h at room temperature. The reaction was quenched by the addition of water/ice (20 mL) at 0° C. The resulting mixture was extracted with CH₂Cl₂ (3×10 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₄HCO₃), 80% to 100% gradient in 10 min; detector: UV 220 nm to afford 6-(4-{1-[(tert-butyldimethylsilyl)oxy]ethenyl}pyrazol-1-yl)-N-(1-methylindazol-7-yl)-N-{[2-(trimethylsilyl)ethoxy]methyl}pyridine-3-sulfonamide (450 mg, 66.51%) as a light yellow oil.

Step 4: Synthesis of 254-4. To a stirred solution of 6-(4-{1-[(tert-butyldimethylsilyl)oxy]ethenyl}pyrazol-1-yl)-N-(1-methylindazol-7-yl)-N-{[2-(trimethylsilyl)ethoxy]methyl}pyridine-3-sulfonamide (500 mg, 0.780 mmol, 1 equiv.) in DCM (30 mL) were added ZnEt₂ (15.60 mL, 15.600 mmol, 20 equiv.) dropwise at 0° C. under a nitrogen atmosphere. The resulting mixture was stirred for an additional 30 min at 0° C. To the above mixture was added CH₂I₂(4178.73 mg, 15.600 mmol, 20 equiv.) dropwise over 5 min at 0° C. The resulting mixture was stirred for an additional 5 h at room temperature. The reaction was quenched by the addition of sat. NH₄Cl (aq.) (20 mL) and water/ice (20 mL) at 0° C. The resulting mixture was extracted with CH₂Cl₂ (3×20 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1 NH₄HCO₃), 90% to 100% gradient in 10 min; detector: UV 220 nm to afford 6-(4-{1-[(tert-butyldimethylsilyl)oxy]cyclopropyl}pyrazol-1-yl)-N-(1-methylindazol-7-yl)-N-{[2-(trimethylsilyl)ethoxy]methyl}pyridine-3-sulfonamide (250 mg, 48.93%) as a light yellow solid.

Step 5: Synthesis of 254-5. To a stirred solution of 6-(4-{1-[(tert-butyldimethylsilyl)oxy]cyclopropyl}pyrazol-1-yl)-N-(1-methylindazol-7-yl)-N-{[2-(trimethylsilyl)ethoxy]methyl}pyridine-3-sulfonamide (140 mg, 0.214 mmol, 1 equiv.) in DCM (10 mL) were added Et₃N·₃HF (10 mL, 73.692 mmol, 344.76 equiv.) dropwise at 0° C. under a nitrogen atmosphere. The resulting mixture was stirred for an additional 6 h at room temperature. The reaction was quenched by the addition of sat. NH₄Cl (aq.) (5 mL) and water/ice (5 mL) at 0° C. The resulting mixture was extracted with CH₂Cl₂ (3×10 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₄HCO₃), 50% to 80% gradient in 10 min; detector: UV 220 nm to afford 6-[4-(1-hydroxycyclopropyl)pyrazol-1-yl]-N-(1-methylindazol-7-yl)-N-{[2-(trimethylsilyl)ethoxy]methyl}pyridine-3-sulfonamide (60 mg, 51.91%) as an off-white solid.

Step 6: Synthesis of 254-6. To a stirred solution of 6-[4-(1-hydroxycyclopropyl)pyrazol-1-yl]-N-(1-methylindazol-7-yl)-N-{[2-(trimethylsilyl)ethoxy]methyl}pyridine-3-sulfonamide (58 mg, 0.107 mmol, 1 equiv.) in DCM (5 mL) was added DAST (35.81 mg, 0.963 mmol, 9 equiv.) dropwise at 0° C. under a nitrogen atmosphere. The resulting mixture was stirred for an additional 2 h at room temperature. The reaction was quenched with sat. NH₄Cl (aq.) (10 mL) at 0° C. The aqueous layer was extracted with CH₂Cl₂ (3×5 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₄HCO₃), 50% to 90% gradient in 10 min; detector: UV 254 nm. To afford 6-[4-(1-fluorocyclopropyl)pyrazol-1-yl]-N-(1-methylindazol-7-yl)-N-{[2-(trimethylsilyl)ethoxy]methyl}pyridine-3-sulfonamide (35 mg, 60.12%) as a light yellow solid.

Step 7: Synthesis of 6-(4-(1-fluorocyclopropyl)-1H-pyrazol-1-yl)-N-(1-methyl-1H-indazol-7-yl)pyridine-3-sulfonamide (I-254). To a stirred solution of 6-[4-(1-fluorocyclopropyl)pyrazol-1-yl]-N-(1-methylindazol-7-yl)-N-{[2-(trimethylsilyl)ethoxy]methyl}pyridine-3-sulfonamide (30 mg, 0.055 mmol, 1 equiv.) in DCM (6 mL) was added TFA (1.5 mL, 20.195 mmol, 365.33 equiv.) dropwise at 0° C. under a nitrogen atmosphere. The resulting mixture was stirred for an additional 4 h at room temperature. The resulting mixture was concentrated under reduced pressure. The residue was purified by reversed-phase flash chromatography with the following conditions: column: C18 silica gel; mobile phase: MeCN in water (10 mmol/L NH₄HCO₃), 30% to 60% gradient in 10 min; detector: UV 254 nm. To afford 6-[4-(1-fluorocyclopropyl)pyrazol-1-yl]-N-(1-methylindazol-7-yl)pyridine-3-sulfonamide (20 mg crude with 90% purity) as a light yellow solid. The crude product (mg) was purified by prep-HPLC with the following conditions: column: XBridge Prep OBD C18, 19*250 mm, 5 μm; mobile phase A: water (10 mmol/L NH₄HCO₃+0.05% NH3H₂O), mobile phase B: MEOH; flow rate 25 mL/min; gradient: 50% B to 70% B in 10 min; wavelength: 254 nm/220 nm; Rt1(min): 8.62; number of runs: 5) to afford 6-[4-(1-fluorocyclopropyl)pyrazol-1-yl]-N-(1-methylindazol-7-yl)pyridine-3-sulfonamide (9.8 mg, 42.98%) as an off-white solid. LCMS: (ES, m/z): [M+H]⁺ 413.20; (400 MHz, DMSO-d₆) δ 1.20-1.09 (m, 2H), 1.49-1.36 (m, 2H), 4.29 (s, 3H), 6.51 (dd, J 7.3, 1.0 Hz, 1H), 6.93 (t, J 7.7 Hz, 1H), 7.69 (d, J 8.0 Hz, 1H), 8.02 (s, 1H), 8.10 (s, 1H), 8.12 (d, J 8.7 Hz, 1H), 8.23 (dd, J 8.7, 2.4 Hz, 1H), 8.62-8.57 (m, 1H), 8.72 (t, J 1.0 Hz, 1H), (400 MHz, DMSO-d₆) δ 1.13-1.17 (m, 2H), 1.39-1.45 (m, 2H), 4.29 (s, 3H), 6.51 (d, J6.8 Hz, 1H), 6.93 (t, J7.6 Hz, 1H), 7.69 (d, J8.0 Hz, 1H), 8.02 (s, 1H), 8.10-8.13 (m, 2H), 8.23 (dd, J8.8, 2.4 Hz, 1H), 8.59 (d, J2.0 Hz, 1H), 8.72 (s, 1H), 10.31 (s, 1H).

### Example 175—Synthesis of (R)-6-(4-Hydroxy-2-Oxo-4-(Trifluoro-Methyl)Pyrrolidin-1-yl)-N-(6-Methoxy-1-Methyl-1H-Pyrazolo[4,3-C]Pyridin-7-yl)Pyridine-3-Sulfonamide (I-235) and (S)-6-(4-Hydroxy-2-Oxo-4-(Trifluoromethyl)Pyrrolidin-1-yl)-N-(6-Methoxy-1-Methyl-1H-Pyrazolo[4,3-C]Pyridin-7-yl)Pyridine-3-Sulfonamide (I-236)

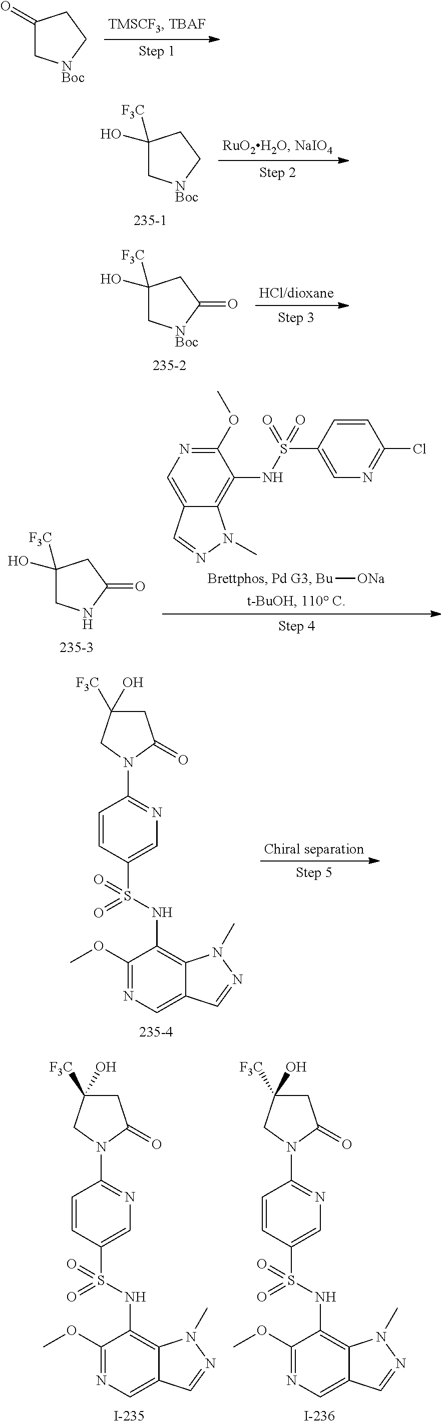

Step 1: Synthesis of 235-1. To a stirred solution of tert-butyl 3-oxopyrrolidine-1-carboxylate (10 g, 53.989 mmol, 1 equiv.) in THF (100 mL) were added TMSCF₃ (40.49 mL, 80.983 mmol, 1.5 equiv.) dropwise at 0° C. under a nitrogen atmosphere. To the above mixture was added TBAF (5.40 mL, 5.399 mmol, 0.1 equiv.) dropwise over 15 min at 0° C. The resulting mixture was stirred for an additional 15 h at room temperature. A new spot could be detected by TLC. To the above mixture was added saturated NH₄Cl aqueous solution (100 mL) dropwise at room temperature. The resulting mixture was stirred for an additional 1 h at room temperature. Finally, to the above mixture was added TBAF (5.40 mL, 5.399 mmol, 0.1 equiv.) dropwise at room temperature. The resulting mixture was stirred for an additional 4 h at room temperature. The resulting mixture was extracted with EtOAc (3×100 mL). The combined organic layers were washed with brine (100 mL) and dried over anhydrous Na₂SO₄. After filtration, the filtrate was concentrated under reduced pressure. The residue was purified by silica gel column chromatography and eluted with PE/EA (5:1) to afford tert-butyl 3-hydroxy-3-(trifluoromethyl)pyrrolidine-1-carboxylate (7 g, 50.80%) as an off-white solid.

Step 2: Synthesis of 235-2. To a stirred mixture of tert-butyl 3-hydroxy-3-(trifluoromethyl)pyrrolidine-1-carboxylate (600 mg, 2.351 mmol, 1 equiv.) and ruthenium(iv) oxide hydrate (35.52 mg, 0.235 mmol, 0.1 equiv.) in EtOAc (30 mL) were added pyridine (18.59 mg, 0.235 mmol, 0.1 equiv.) dropwise at room temperature under a nitrogen atmosphere. To the above mixture was added a solution of NaIO4 (2011.21 mg, 9.404 mmol, 4.0 equiv.) in H₂O (7.(5 mL) dropwise over 10 min at room temperature. The resulting mixture was stirred for an additional 15 h at 50° C. The reaction was monitored by LCMS and TLC. The resulting mixture was filtered, the filter cake was washed with ethyl acetate (3×10 mL). The combined organic layers were washed with brine (20 mL) and dried over anhydrous Na₂SO₄. After filtration, the filtrate was concentrated under reduced pressure. The residue was purified by silica gel column chromatography, eluted with PE/EA (5:1) to afford tert-butyl 4-hydroxy-2-oxo-4-(trifluoromethyl)pyrrolidine-1-carboxylate (400 mg, 63.20%) as a light yellow oil.

Step 3: Synthesis of 235-3. To a stirred solution of tert-butyl 4-hydroxy-2-oxo-4-(trifluoromethyl)pyrrolidine-1-carboxylate (300 mg, 1.114 mmol, 1 equiv.) in 1,4-dioxane (6 ml) was added a solution of HCl (gas) in 1,4-dioxane (6 mL, 24.000 mmol, 21.54 equiv.) dropwise at 0° C. under a nitrogen atmosphere. The resulting mixture was stirred for an additional 4 h at room temperature. The resulting mixture was concentrated under reduced pressure to afford 4-hydroxy-4-(trifluoromethyl)pyrrolidin-2-one (185 mg, 98.18%) as a light yellow oil. The crude product was used in the next step directly without further purification.

Step 4: Synthesis of 235-4. To a stirred mixture of 4-hydroxy-4-(trifluoromethyl)pyrrolidin-2-one (86.04 mg, 0.509 mmol, 1.5 equiv.) and 6-chloro-N-{6-methoxy-1-methylpyrazolo[4,3-c]pyridin-7-yl}pyridine-3-sulfonamide (120 mg, 0.339 mmol, 1.00 equiv.) in t-BuOH (10 mL) were added BrettPhos Pd G3 (30.75 mg, 0.034 mmol, 0.1 equiv.) and t-BuONa (48.90 mg, 0.509 mmol, 1.50 equiv.) at room temperature under a nitrogen atmosphere. The resulting mixture was stirred for an additional 4 h at 110° C. The reaction was quenched by the addition of sat. NH₄Cl (aq.) (5 mL) and water/ice (5 mL) at 0° C. The resulting mixture was extracted with CH₂Cl₂ (3×5 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₄HCO₃), 30% to 60% gradient in 10 min; detector: UV 254 nm. To afford 6-[4-hydroxy-2-oxo-4-(trifluoromethyl)pyrrolidin-1-yl]-N-{6-methoxy-1-methylpyrazolo[4,3-c]pyridin-7-yl}pyridine-3-sulfonamide (40 mg, 24.24%) as an off-white solid.

Step 5: Synthesis of (R)-6-(4-hydroxy-2-oxo-4-(trifluoromethyl)pyrrolidin-1-yl)-N-(6-methoxy-1-methyl-1H-pyrazolo[4,3-c]pyridin-7-yl)pyridine-3-sulfonamide (I-235) and (S)-6-(4-hydroxy-2-oxo-4-(trifluoromethyl)pyrrolidin-1-yl)-N-(6-methoxy-1-methyl-11H-pyrazolo[4,3-c]pyridin-7-yl)pyridine-3-sulfonamide (I-236). The racemic product (40 mg) was separated by Chiral-HPLC with the following conditions: column: CHIRALPAK IA, 2*25 cm, 5 μm; mobile phase A: hex (0.5% 2M NH3-MeOH), mobile phase B: EtOH:DCM=1:1-HPLC; flow rate 20 mL/min; gradient: 20% B to 20% B in 20 min; wavelength: 220/254 nm; Rt1(min): 11.08; Rt2(min): 16.89; Sample Solvent: EtOH:DCM=1:1; injection volume: 0.64 mL; number of runs: 5) to afford (R)-6-(4-hydroxy-2-oxo-4-(trifluoromethyl)pyrrolidin-1-yl)-N-(6-methoxy-1-methyl-1H-pyrazolo[4,3-c]pyridin-7-yl)pyridine-3-sulfonamide (11.9 mg) as an off-white solid and (S)-6-(4-hydroxy-2-oxo-4-(trifluoromethyl)pyrrolidin-1-yl)-N-(6-methoxy-1-methyl-1H-pyrazolo[4,3-c]pyridin-7-yl)pyridine-3-sulfonamide (10.4 mg) as an off-white solid. LCMS: (ES, m/z): [M+H]⁺ 487.15; ¹H NMR (400 MHz, DMSO-d₆) δ 2.74-2.78 (d, J 17.2 Hz, 1H), 3.25-3.29 (d, J 17.2 Hz, 1H), 3.29 (s, 3H), 4.22 (s, 2H), 4.23 (s, 3H), 7.00 (s, 1H), 8.09 (dd, J 9.2, 2.4 Hz, 1H), 8.22 (s, 1H), 8.45 (d, J8.8 Hz, 1H), 8.56 (d, J2.4 Hz, 1H), 8.65 (s, 1H), 10.01 (s, 1H).

### Example 176—Synthesis of N-(6-Methoxy-1-Methyl-1H-Indazol-7-yl)-6-(4-Methyl-2-Oxo-2,3-Dihydro-1H-Imidazol-1-yl)Pyridine-3-Sulfonamide (I-237)

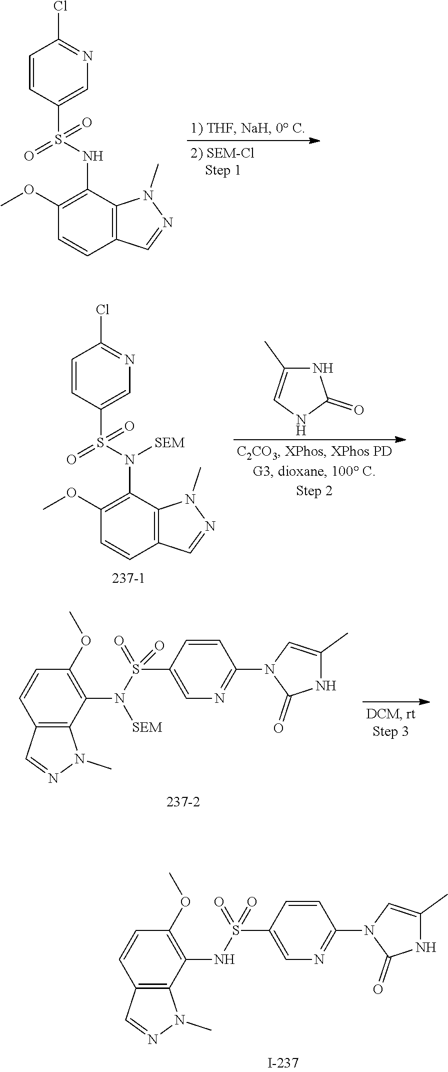

Step 1: Synthesis of 237-1. A solution of 6-chloro-N-(6-methoxy-1-methylindazol-7-yl)pyridine-3-sulfonamide (1.1 g, 3.118 mmol, 1 equiv.) in tetrahydrofuran (25 mL) was treated with NaH (154 mg, 3.850 mmol, 1.23 equiv., 60%) for 30 min at 0° C. under a nitrogen atmosphere followed by the addition of [2-(chloromethoxy)ethyl]trimethylsilane (1.15 mL, 6.291 mmol, 2.02 equiv.) dropwise at 0° C. The resulting mixture was stirred for 2 h at room temperature under a nitrogen atmosphere. The resulting mixture was filtered and the filter cake was washed with EtOAc (3×25 mL). The filtrate was concentrated under reduced pressure. The residue was purified by reversed-phase flash chromatography with the following conditions: column: C18 silica gel; mobile phase: MeCN in water (10 mmol/L NH₄HCO₃), 90% to 95% gradient in 30 min; detector: UV 220 nm. This resulted in 237-1 (0.7 g, 46.48%) as a brown solid.

Step 2: Synthesis of 237-2. To a stirred solution of 237-1 (300 mg, 0.621 mmol, 1 equiv.), Cs₂CO₃ (600 mg, 1.842 mmol, 2.97 equiv.), XPhos (60 mg, 0.126 mmol, 0.20 equiv.) and XPhos Pd G3 (110 mg, 0.130 mmol, 0.21 equiv.) in dioxane (12 mL) was added 4-methyl-1,3-dihydroimidazol-2-one (80 mg, 0.815 mmol, 1.31 equiv.) in portions at room temperature under a nitrogen atmosphere. The resulting mixture was stirred for 1 h at 100° C. under a nitrogen atmosphere. The resulting mixture was filtered and the filter cake was washed with MeCN (3×10 mL). The filtrate was concentrated under reduced pressure. The residue was purified by reversed-phase flash chromatography with the following conditions: column: C18 silica gel; mobile phase: MeCN in water (10 mmol/L NH₄HCO₃), 60% to 65% gradient in 10 min; detector: UV 210 nm. This resulted in 237-2 (180 mg, 53.21%) as a brown solid.

Step 3: Synthesis of N-(6-methoxy-1-methyl-1H-indazol-7-yl)-6-(4-methyl-2-oxo-2,3-dihydro-1H-imidazol-1-yl)pyridine-3-sulfonamide (I-237). A solution of 237-2 (160 mg, 0.294 mmol, 1 equiv.) and TFA (3 mL, 40.389 mmol, 137.50 equiv.) in DCM (3 mL) was stirred for 0.5 h at room temperature under a nitrogen atmosphere. The resulting mixture was concentrated under reduced pressure. The residue was dissolved in DCM (3 mL) and neutralized to pH 7 with saturated NaHCO₃ (aq.) at 0° C. The resulting mixture was extracted with DCM (3×3 mL). The combined organic layers were washed with brine (5 mL) and dried over anhydrous Na₂SO₄. After filtration, the filtrate was concentrated under reduced pressure. The crude product (30 mg) was purified by prep-HPLC with the following conditions: column: Xselect CSH C18 OBD, 30*150 mm 5 m; mobile phase A: water(0.1% FA), mobile phase B: ACN; flow rate 60 mL/min; gradient: 25% B to 35% B in 8 min; wavelength: 254 nm/220 nm; Rt1(min): 7.28) to afford N-(6-methoxy-1-methyl-1H-indazol-7-yl)-6-(4-methyl-2-oxo-2,3-dihydro-1H-imidazol-1-yl)pyridine-3-sulfonamide (11.2 mg, 8.51%) as a white solid. LCMS: (ES, m/z): [M+H]⁺ 415; (400 MHz, DMSO-d₆): δ 2.00 (s, 3H), 3.17 (s, 3H), 4.27 (s, 3H), 6.80 (d, J9.2 Hz, 1H), 7.05 (s, 1H), 7.61 (d, J8.8 Hz, 1H), 7.96 (s, 1H), 8.06 (dd, J8.8, 2.4 Hz, 1H), 8.48-8.51 (m, 2H), 10.00 (s, 1H), 10.54 (s, 1H).

### Example 177—Synthesis of Ethyl 1-(5-(N-(6-Methoxy-1-Methyl-1H-Indazol-7-yl)Sulfamoyl)Pyridin-2-yl)-5-Methyl-1H-Pyrazole-4-Carboxylate (1-238)

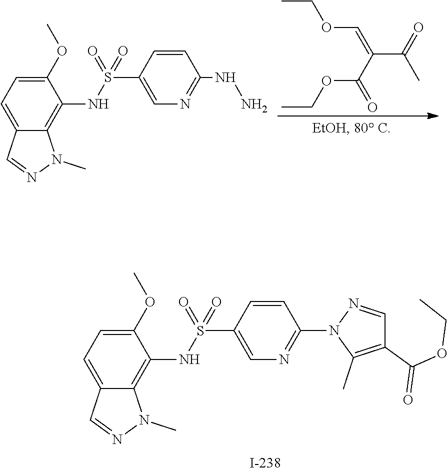

A mixture of 6-hydrazinyl-N-(6-methoxy-1-methylindazol-7-yl)pyridine-3-sulfonamide (100 mg, 0.287 mmol, 1 equiv.) and ethyl (2Z)-2-(ethoxymethylidene)-3-oxobutanoate (80.17 mg, 0.430 mmol, 1.5 equiv.) in EtOH (2 mL) was stirred for 2 hours at 80° C. The resulting mixture was diluted with water (20 mL). The resulting mixture was extracted with CH₂Cl₂ (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water, 10% to 50% gradient in 10 min; detector: UV 254 nm. This resulted in ethyl 1-{5-[(6-methoxy-1-methylindazol-7-yl)sulfamoyl]pyridin-2-yl}-5-methylpyrazole-4-carboxylate (55.4 mg, 40.65%) as an off-white solid. LCMS: (ES, m/z): [M+H]⁺ 471; (400 MHz, DMSO-d₆) δ 1.31 (t, J 7.2 Hz, 3H), 2.90 (s, 3H), 3.21 (s, 3H), 4.01-4.59 (m, 5H), 6.84 (d, J 8.8 Hz, 1H), 7.69 (d, J 8.8 Hz, 1H), 8.00 (s, 1H), 8.07 (d, J 8.4 Hz, 1H), 8.15 (s, 1H), 8.26 (dd, J=8.4, 2.4 Hz, 1H), 8.71 (d, J 2.4 Hz, 1H), 10.10 (s, 1H).

### Example 178—Synthesis of 6-(1,3-Dimethyl-1H-1,2,4-Triazol-5-yl)-N-(6-Methoxy-1-Methyl-1H-Indazol-7-yl)Pyridine-3-Sulfonamide (I-239)

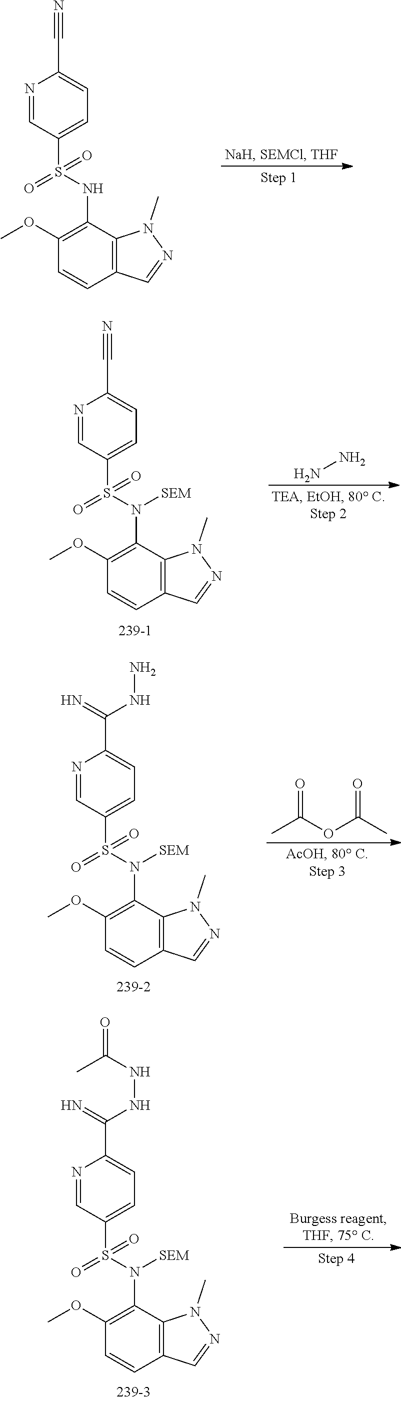

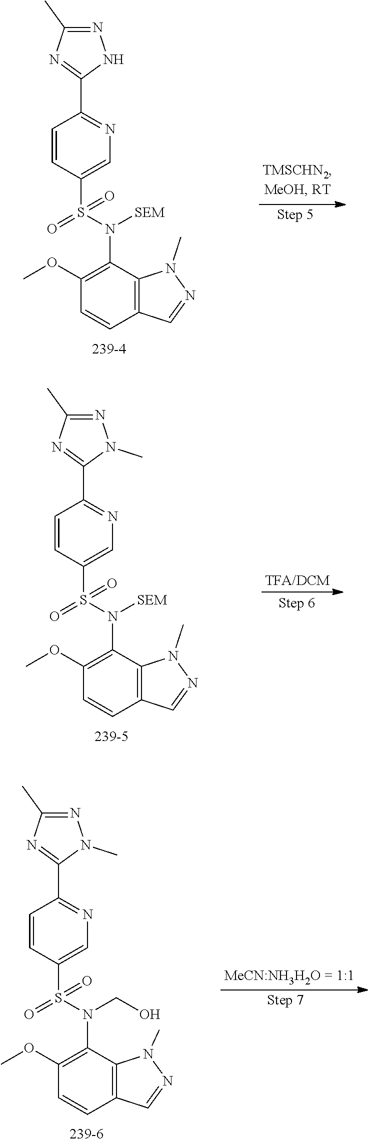 [SMILES: COc1ccc2cnn(C)c2c1N(COCC[Si](C)(C)C)S(=O)(=O)c1ccc(-c2nc(C)n[nH]2)nc1 ; COc1ccc2cnn(C)c2c1N(COCC[Si](C)(C)C)S(=O)(=O)c1ccc(-c2nc(C)nn2C)nc1 ; COc1ccc2cnn(C)c2c1N(CO)S(=O)(=O)c1ccc(-c2nc(C)nn2C)nc1]

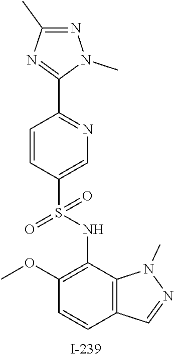 [SMILES: COc1ccc2cnn(C)c2c1NS(=O)(=O)c1ccc(-c2nc(C)nn2C)nc1]

Step 1: Synthesis of 239-1. A solution of 6-cyano-N-(6-methoxy-1-methylindazol-7-yl)pyridine-3-sulfonamide (800 mg, 2.330 mmol, 1 equiv.) in THF (16 mL) was treated with NaH (167.74 mg, 6.990 mmol, 3 equiv.) for 30 min under a nitrogen atmosphere followed by the addition of SEMCl (1165.33 mg, 6.990 mmol, 3 equiv.) dropwise/in portions at 0° C. The mixture was stirred for 3 h at room temperature under a nitrogen atmosphere. The reaction was quenched by the addition of sat. NH₄Cl (aq.) (20 mL) at 0° C. The resulting mixture was extracted with EtOAc (2×50 mL). The combined organic layers were washed with water (10 mL) and dried over anhydrous Na₂SO₄. After filtration, the filtrate was concentrated under reduced pressure. The crude product 6-cyano-N-(6-methoxy-1-methylindazol-7-yl)-N-{[2-(trimethylsilyl)ethoxy]methyl}pyridine-3-sulfonamide (1.1 g, 99.68%) was used in the next step directly without further purification.

Step 2: Synthesis of 239-2. A mixture of 6-cyano-N-(6-methoxy-1-methylindazol-7-yl)-N-{[2-(trimethylsilyl)ethoxy]methyl}pyridine-3-sulfonamide (1.5 g, 3.167 mmol, 1 equiv.), TEA (0.96 g, 9.501 mmol, 3 equiv.) and hydrazine (30.45 g, 950.100 mmol, 300 equiv.) in EtOH (15 mL) was stirred for 2 h at 80° C. under a nitrogen atmosphere. The resulting mixture was concentrated under reduced pressure. The residue was purified by reversed-phase flash chromatography with the following conditions: column: C18 silica gel; mobile phase: MeCN in water (0.1% FA), 65% to 75% gradient in 10 min; detector: UV 254 nm to afford N-amino-5-[(6-methoxy-1-methylindazol-7-yl)({[2-(trimethylsilyl)ethoxy]methyl})sulfamoyl]pyridine-2-carboximidamide (1 g, 62.44%) as a yellow solid.

Step 3: Synthesis of 239-3. A solution of N-amino-5-[(6-methoxy-1-methylindazol-7-yl)({[2-(trimethylsilyl)ethoxy]methyl})sulfamoyl]pyridine-2-carboximidamide (600 mg, 1.187 mmol, 1 equiv.) in HOAc (6 mL) was treated with acetic anhydride (363.40 mg, 3.561 mmol, 3 equiv.) and p-toluenesulfonic acid (204.32 mg, 1.187 mmol, 1 equiv.) at 0° C. The resulting mixture was stirred for 2 h at 50° C. The reaction was quenched by the addition of sat. Na₂CO₃ (aq.) (10 mL) at 0° C. The resulting mixture was extracted with EtOAc (2×50 mL). The combined organic layers were washed with water (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 60% gradient in 10 min; detector: UV 254 nm to afford 6-(acetohydrazidomethanimidoyl)-N-(6-methoxy-1-methylindazol-7-yl)-N-{[2-(trimethylsilyl)ethoxy]methyl}pyridine-3-sulfonamide (500 mg, 76.94%) as a yellow solid.

Step 4: Synthesis of 239-4. A solution of 6-(acetohydrazidomethanimidoyl)-N-(6-methoxy-1-methylindazol-7-yl)-N-{[2-(trimethylsilyl)ethoxy]methyl}pyridine-3-sulfonamide (380 mg, 0.694 mmol, 1 equiv.) in THE (20 mL) was treated with Burgess reagent (496.00 mg, 2.082 mmol, 3 equiv.). The resulting mixture was stirred for 3 h at 50° C. The reaction was quenched by the addition of water (10 mL) at 0° C. The resulting mixture was extracted with EtOAc (2×50 mL). The combined organic layers were washed with water (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 40% to 60% gradient in 10 min; detector: UV 254 nm to afford N-(6-methoxy-1-methylindazol-7-yl)-6-(5-methyl-2H-1,2,4-triazol-3-yl)-N-{[2-(trimethylsilyl)ethoxy]methyl}pyridine-3-sulfonamide (140 mg, 38.10%) as a yellow solid.

Step 5: Synthesis of 239-5. A solution of N-(6-methoxy-1-methylindazol-7-yl)-6-(5-methyl-2H-1,2,4-triazol-3-yl)-N-{[2-(trimethylsilyl)ethoxy]methyl}pyridine-3-sulfonamide (140 mg, 0.264 mmol, 1 equiv.) in MeOH (6 mL) at 0° C. was treated with TMSCHN₂ (301.90 mg, 2.640 mmol, 10 equiv.). The resulting mixture was stirred for 3 h at room temperature. The resulting mixture was diluted with water (10 mL). The resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 60% to 70% gradient in 10 min; detector: UV 254 nm to afford 6-(2,5-dimethyl-1,2,4-triazol-3-yl)-N-(6-methoxy-1-methylindazol-7-yl)-N-{[2-(trimethylsilyl)ethoxy]methyl}pyridine-3-sulfonamide (40 mg, 27.83%) as a yellow solid.

Step 6: Synthesis of 239-6. A solution of 6-(2,5-dimethyl-1,2,4-triazol-3-yl)-N-(6-methoxy-1-methylindazol-7-yl)-N-{[2-(trimethylsilyl)ethoxy]methyl}pyridine-3-sulfonamide (40 mg, 0.074 mmol, 1 equiv.) in DCM (2 mL) at 0° C. was treated with TFA (83.88 mg, 0.740 mmol, 10 equiv.). The resulting mixture was stirred for 2 h at room temperature. The resulting mixture was concentrated under reduced pressure. The crude product 6-(2,5-dimethyl-1,2,4-triazol-3-yl)-N-(hydroxymethyl)-N-(6-methoxy-1-methylindazol-7-yl)pyridine-3-sulfonamide (32 mg, 98.08%) was obtained as a yellow oil and was used in the next step directly without further purification.

Step 7: Synthesis of 6-(1,3-dimethyl-1H-1,2,4-triazol-5-yl)-N-(6-methoxy-1-methyl-1H-indazol-7-yl)pyridine-3-sulfonamide (I-239). A solution/mixture of 6-(2,5-dimethyl-1,2,4-triazol-3-yl)-N-(hydroxymethyl)-N-(6-methoxy-1-methylindazol-7-yl)pyridine-3-sulfonamide (30 mg, 0.068 mmol, 1 equiv.) in MeCN (3 mL) and NH₃·H₂O (3 mL) was stirred for 1 h at room temperature under a nitrogen atmosphere. The resulting mixture was concentrated under reduced pressure. The residue was purified by reversed-phase flash chromatography with the following conditions: column: C18 silica gel; mobile phase: MeCN in water (0.1% FA), 30% to 40% gradient in 10 min; detector: UV 254 nm to afford 6-(2,5-dimethyl-1,2,4-triazol-3-yl)-N-(6-methoxy-1-methylindazol-7-yl)pyridine-3-sulfonamide (21.1 mg, 72.05%) as a white solid. LCMS: (ES, m/z): [M+H]⁺ 414; (400 MHz, DMSO-d₆) δ 2.33 (s, 3H), 3.13 (s, 3H), 4.22 (s, 3H), 4.28 (s, 3H), 6.82 (d, J8.8 Hz, 1H), 7.66 (d, J 8.8 Hz, 1H), 7.99 (s, 1H), 8.18 (dd, J8.4, 2.0 Hz, 1H), 8.28 (d, J8.4 Hz, 1H), 8.88 (d, J 1.6 Hz, 1H), 10.25 (s, 1H).

### Example 179—Synthesis of 6-(4-Chloro-1H-Pyrazol-1-yl)-N-(3-Fluoro-6-Methoxy-1-Methyl-1H-Indazol-7-yl)Pyridine-3-Sulfonamide (I-240)

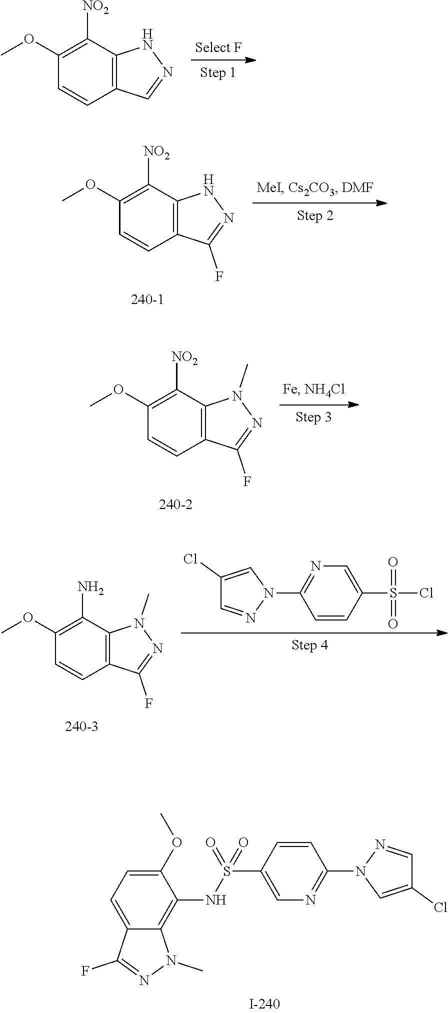 [SMILES: COc1ccc2cn[nH]c2c1[N+](=O)[O-] ; COc1ccc2c(F)n[nH]c2c1[N+](=O)[O-] ; COc1ccc2c(F)nn(C)c2c1[N+](=O)[O-] ; COc1ccc2c(F)nn(C)c2c1N ; O=S(=O)(Cl)c1ccc(-n2cc(Cl)cn2)nc1 ; COc1ccc2c(F)nn(C)c2c1NS(=O)(=O)c1ccc(-n2cc(Cl)cn2)nc1]

Step 1: Synthesis of 240-1. A solution of 6-methoxy-7-nitro-1H-indazole (8 g, 41.416 mmol, 1 equiv.) and Selectfluor (14.67 g, 41.410 mmol, 1.00 equiv.) in MeCN (1000 mL) was stirred overnight at 80° C. under a nitrogen atmosphere. The resulting liquid was dried under vacuum. The resulting mixture was diluted with water (20 mL). The resulting mixture was extracted with EtOAc (3×200 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 30% to 60% gradient in 10 min; detector: UV 254 nm. This resulted in 240-1 (3.08 g, 35.22%) as a yellow solid.

Step 2: Synthesis of 240-2. A solution of 3-fluoro-6-methoxy-7-nitro-1H-indazole (3.07 g, 14.539 mmol, 1 equiv.) in DMF (15 mL) was treated with NaH (0.70 g, 29.078 mmol, 2 equiv.) for 10 min a 0° C. under a nitrogen atmosphere followed by the addition of Mel (3.10 g, 21.840 mmol, 1.50 equiv.) dropwise at room temperature. The resulting mixture was stirred for 2 h at room temperature under a nitrogen atmosphere. The reaction was quenched with sat. NH₄Cl (aq.) at room temperature and then extracted with EtOAc (3×100 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 50% to 70% gradient in 10 min; detector: UV 254 nm. This resulted in 240-2 (550 mg, 16.80%) as a yellow solid.

Step 3: Synthesis of 240-3. A mixture of 3-fluoro-6-methoxy-1-methyl-7-nitroindazole (240 mg, 1.066 mmol, 1 equiv.) and Fe (300 mg, 5.372 mmol, 5.04 equiv.) and NH₄Cl (300 mg, 5.609 mmol, 5.26 equiv.) in EtOH (2 mL) was stirred for 2 h at 80° C. under a nitrogen atmosphere. The resulting mixture was filtered and the filter cake was washed with EtOH (5×10 mL). The filtrate was concentrated under reduced pressure. The residue was purified by reversed-phase flash chromatography with the following conditions: column: C18 silica gel; mobile phase: MeCN in water (10 mmol/L NH₄HCO₃), 35% to 60% gradient in 10 min; detector: UV 254 nm. This resulted in 240-3 (120 mg, 57.68%) as a brown oil.

Step 4: 6-(4-chloro-1H-pyrazol-1-yl)-N-(3-fluoro-6-methoxy-1-methyl-1H-indazol-7-yl)pyridine-3-sulfonamide (1-240). A solution of 3-fluoro-6-methoxy-1-methylindazol-7-amine (100 mg, 0.512 mmol, 1 equiv.) in THF (10 mL) was treated with LDA (110 mg, 1.027 mmol, 2.00 equiv.) for 30 min at −78° C. under a nitrogen atmosphere followed by the addition of 6-(4-chloropyrazol-1-yl)pyridine-3-sulfonyl chloride (128 mg, 0.460 mmol, 0.90 equiv.) dropwise at room temperature. The resulting mixture was stirred for 2 h at room temperature under a nitrogen atmosphere. The resulting mixture was diluted with water (10 mL). The resulting mixture was extracted with EtOAc (3×20 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in 6-(4-chloro-1H-pyrazol-1-yl)-N-(3-fluoro-6-methoxy-1-methyl-1H-indazol-7-yl)pyridine-3-sulfonamide as a brown solid. LCMS: (ES, m/z): [M+H]⁺ 437; ¹H NMR (400 MHz, DMSO-d₆) δ 3.40 (s, 3H), 4.29 (s, 3H), 7.64 (d, J 10.4 Hz, 1H), 8.04 (s, 1H), 8.09 (s, 1H), 8.10 (d, J8.8 Hz, 1H), 8.32 (dd, J 8.8, 2.4 Hz, 1H), 8.72 (d, J2.0 Hz, 1H), 8.93 (s, 1H).

### Example 180—Synthesis of 6-(2-(Difluoromethyl)Thiazol-5-yl)-N-(1-Methyl-1H-Indazol-7-yl)Pyridine-3-Sulfonamide (I-241)

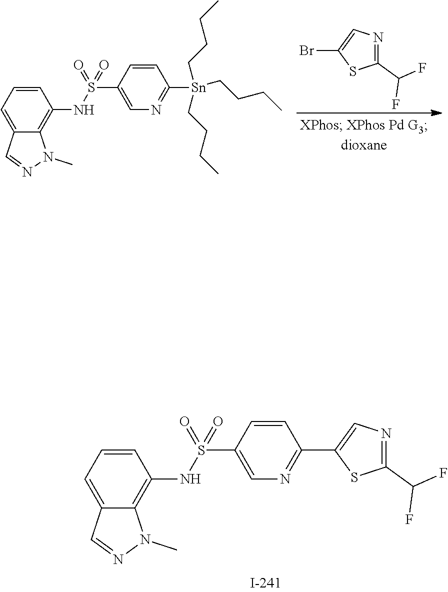 [SMILES: CCC[CH2][Sn]([CH2]CCC)([CH2]CCC)[c]1ccc(S(=O)(=O)Nc2cccc3cnn(C)c23)cn1 ; FC(F)c1ncc(Br)s1 ; Cn1ncc2cccc(NS(=O)(=O)c3ccc(-c4cnc(C(F)F)s4)nc3)c21]

A solution of N-(1-methyl-1H-indazol-7-yl)-6-(tributylstannyl)pyridine-3-sulfonamide (100 mg, 0.173 mmol, 1.00 equiv.), 5-bromo-2-(difluoromethyl)-1,3-thiazole (44.4 mg, 0.208 mmol, 1.20 equiv.), XPhos (16.51 mg, 0.035 mmol, 0.20 equiv.) and XPhos Pd G3 (14.6 mg, 0.017 mmol, 0.10 equiv.) in dioxane (4 mL) was stirred for 2 h at 90° C. under a nitrogen atmosphere. The mixture was allowed to cool down to room temperature. The resulting mixture was diluted with water (20 mL). The resulting mixture was extracted with EtOAc (3×10 mL). The combined organic layers were washed with brine (15 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃)+0.05% NH₃·H₂O, mobile phase B: ACN; flow rate 60 mL/min; gradient: 13% B to 40% B in 7 min; wavelength: 254 nm/220 nm; Rt1(min): 8.28) to afford 6-(2-(difluoromethyl)thiazol-5-yl)-N-(1-methyl-1H-indazol-7-yl)pyridine-3-sulfonamide (20.8 mg, 28.04%) as an off-white solid. (ES, m/z): [M+H]⁺422; 1H NMR (400 MHz, DMSO-d₆) δ 4.30 (s, 3H), 6.57 (d, J 7.2 Hz, 1H), 6.92 (t, J 7.2 Hz, 1H), 7.40 (t, J 54.0 Hz, 1H), 7.63 (d, J 8.0 Hz, 1H), 8.07 (s, 1H), 8.17 (dd, J 8.4, 2.4 Hz, 1H), 8.33 (d, J 8.4 Hz, 1H), 8.82 (s, 1H), 8.85 (s, 1H), 10.36 (s, 1H).

### Example 181—Synthesis of 6-(4-(Hydroxymethyl)-5-Methyl-1H-Pyrazol-1-yl)-N-(6-Methoxy-1-Methyl-1H-Indazol-7-yl)Pyridine-3-Sulfonamide (I-242)

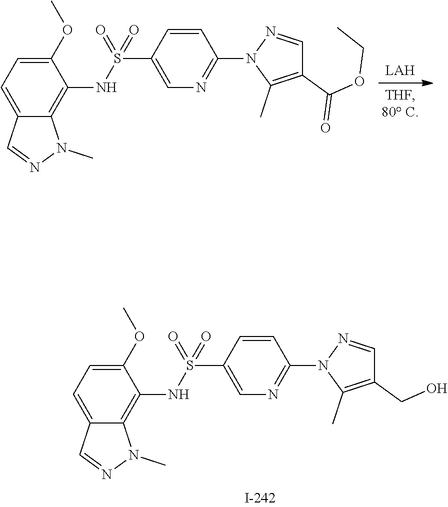 [SMILES: CCOC(=O)c1cnn(-c2ccc(S(=O)(=O)Nc3c(OC)ccc4cnn(C)c34)cn2)c1C ; COc1ccc2cnn(C)c2c1NS(=O)(=O)c1ccc(-n2ncc(CO)c2C)nc1]

A mixture of ethyl 1-{5-[(6-methoxy-1-methylindazol-7-yl)sulfamoyl]pyridin-2-yl}-5-methylpyrazole-4-carboxylate (150 mg, 0.319 mmol, 1 equiv.) and LAH (24.20 mg, 0.638 mmol, 2 equiv.) in THF (4 mL) was stirred for 3 h at room temperature under a nitrogen atmosphere. The resulting mixture was quenched with water (20 mL). The resulting mixture was extracted with EtOAc (3×10 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The crude product (100 mg) was purified by prep-HPLC with the following conditions: column: XBridge Prep OBD C18, 30*150 mm, 5 m; mobile phase A: water (10 mmol/L NH₄HCO₃)+0.05% NH3. H₂O, mobile phase B: ACN; flow rate 60 mL/min; gradient: 10% B to 35% B in 7 min; wavelength: 254 nm/220 nm; Rt1(min): 8.08) to afford 6-[4-(hydroxymethyl)-5-methylpyrazol-1-yl]-N-(6-methoxy-1-methylindazol-7-yl)pyridine-3-sulfonamide (29.4 mg, 21.31%) as a white solid. LCMS: (ES, m/z): [M+H]⁺ 429; ¹H NMR (400 MHz, DMSO-d₆) δ 2.62 (s, 3H), 3.20 (s, 3H), 4.27 (s, 3H), 4.39 (d, J 5.2 Hz, 2H), 4.88 (t, J 5.2 Hz, 1H), 6.83 (d, J 8.8 Hz, 1H), 7.67 (d, J 8.8 Hz, 1H), 7.73 (s, 1H), 7.99 (s, 1H), 8.04 (d, J 8.8 Hz, 1H), 8.16 (dd, J8.8, 2.4 Hz, 1H), 8.62 (d, J2.0 Hz, 1H), 10.00 (s, 1H).

### Example 182—Synthesis of 6-(2-Methoxythiazol-5-yl)-N-(1-Methyl-1H-Indazol-7-yl)Pyridine-3-Sulfonamide (I-243)

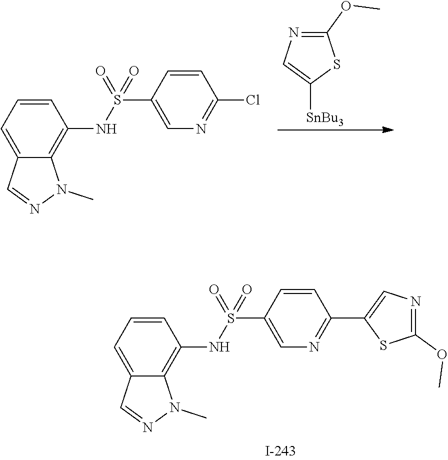

A mixture of 6-chloro-N-(1-methyl-1H-indazol-7-yl)pyridine-3-sulfonamide (150.00 mg, 0.465 mmol, 1 equiv.), 2-methoxy-5-(tributylstannyl)thiazole (225.41 mg, 0.558 mmol, 1.2 equiv.), XPhos (44.31 mg, 0.093 mmol, 0.2 equiv.) and XPhos Pd G3 (78.67 mg, 0.093 mmol, 0.2 equiv.) in dioxane (3 mL) was stirred for 2 h at 90° C. under a nitrogen atmosphere. The resulting mixture was diluted with water (15 mL) and extracted with EtOAc (3×15 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 10% to 50% gradient in 10 min; detector: UV 254 nm, to afford 6-(2-methoxythiazol-5-yl)-N-(1-methyl-1H-indazol-7-yl)pyridine-3-sulfonamide (60.40 mg, 31.69%) as an off-white solid. LCMS: (ES, m/z): [M+H]⁺ 401; (400 MHz, DMSO-d₆) δ 4.10 (s, 3H), 4.29 (s, 3H), 6.52 (d, J 7.2 Hz,1H), 6.93 (t, J 7.6 Hz, 1H), 7.66 (d, J 8.0 Hz, 1H), 8.04 (dd, J 8.8, 2.4 Hz, 1H), 8.08 (s, 1H), 8.13(d, J 8.4 Hz, 1H), 8.16 (s, 1H), 8.66 (d, J 2.4 Hz, 1H), 10.29 (s, 1H).

### Example 183—Synthesis of N-(1-Methyl-1H-Indazol-7-yl)-6-(2-Morpholinothiazol-5-yl)Pyridine-3-Sulfonamide (I-244)

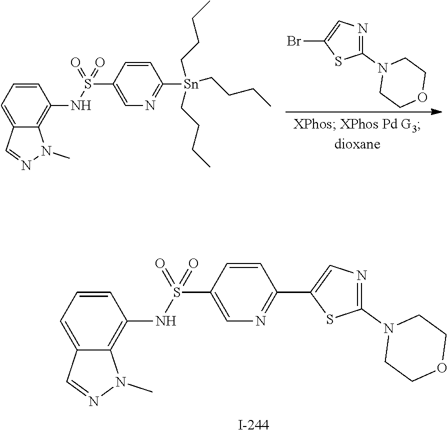 [SMILES: CCC[CH2][Sn]([CH2]CCC)([CH2]CCC)[c]1ccc(S(=O)(=O)Nc2cccc3cnn(C)c23)cn1 ; Brc1cnc(N2CCOCC2)s1 ; Cn1ncc2cccc(NS(=O)(=O)c3ccc(-c4cnc(N5CCOCC5)s4)nc3)c21]

A solution of N-(1-methyl-1H-indazol-7-yl)-6-(tributylstannyl)pyridine-3-sulfonamide (100 mg, 0.173 mmol, 1.00 equiv.), 4-(5-bromo-1,3-thiazol-2-yl)morpholine (51.7 mg, 0.208 mmol, 1.20 equiv.), XPhos (16.5 mg, 0.035 mmol, 0.20 equiv.) and XPhos Pd G3 (14.6 mg, 0.017 mmol, 0.10 equiv.) in dioxane (4 mL) was stirred for 2 h at 90° C. under a nitrogen atmosphere. The mixture was allowed to cool down to room temperature. The resulting mixture was diluted with water (20 mL). The resulting mixture was extracted with EtOAc (3×10 mL). The combined organic layers were washed with brine (15 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃)+0.05% NH₃·H₂O, mobile phase B: ACN; flow rate 60 mL/min; gradient: 10% B to 35% B in 7 min; wavelength: 254 nm/220 nm; Rt1(min): 8.18) to affordN-(1-methyl-1H-indazol-7-yl)-6-(2-morpholinothiazol-5-yl)pyridine-3-sulfonamide (3.9 mg, 4.91%) as an off-white solid. (ES, m/z): [M+H]⁺ 457; ¹H NMR (DMSO-d₆, 400 MHz): δ 3.45 (t, J 4.8 Hz, 4H), 3.66 (t, J 4.8 Hz, 4H), 4.21 (s, 3H), 6.47 (dd, J 7.2, 1.2 Hz, 1H), 6.86 (t, J 7.6 Hz, 1H), 7.57 (d, J 8.0 Hz, 1H), 7.85 (dd, J 8.8, 2.4 Hz, 1H), 7.90 (dd, J 8.8, 2.4 Hz, 1H), 8.00 (s, 1H), 8.07 (s, 1H), 8.50 (dd, J 2.4, 0.8 Hz, 1H), 10.12 (s, 1H).

### Example 184—Synthesis of 6-(4-(1-Hydroxycyclopropyl)-1H-Pyrazol-1-yl)-N-(1-Methyl-1H-Indazol-7-yl)Pyridine-3-Sulfonamide (I-56)

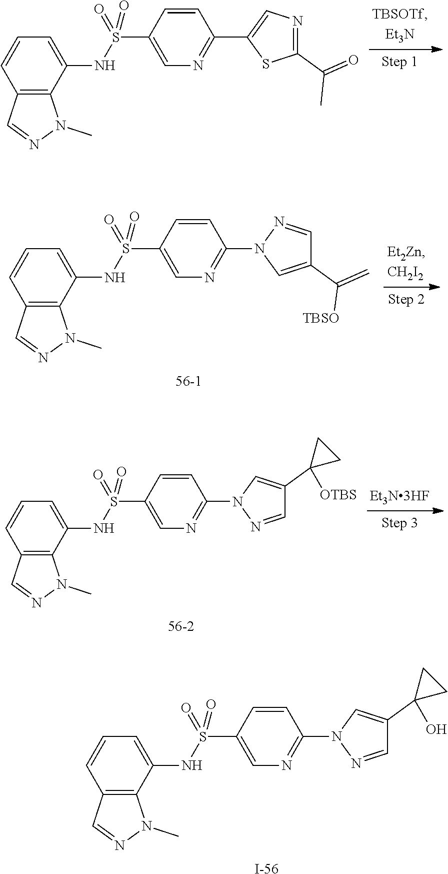

Step 1: Synthesis of 56-1. To a stirred solution of 6-(4-acetylpyrazol-1-yl)-N-(1-methylindazol-7-yl)pyridine-3-sulfonamide (200 mg, 0.505 mmol, 1 equiv.) and Et₃N (153.16 mg, 1.515 mmol, 3.0 equiv.) in DCM (10 mL) were added TBSOTf (200.03 mg, 0.758 mmol, 1.5 equiv.) dropwise at 0° C. under a nitrogen atmosphere. The resulting mixture was stirred for an additional hour at room temperature. The reaction was quenched by the addition of water/ice (10 mL) at 0° C. The resulting mixture was extracted with EtOAc (3×mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 70% to 90% gradient in 10 min; detector: UV 220 nm. To afford 6-(4-{1-[(tert-butyldimethylsilyl)oxy]ethenyl}pyrazol-1-yl)-N-(1-methylindazol-7-yl)pyridine-3-sulfonamide (140 mg, 54.34%) as a light yellow solid.

Step 2: Synthesis of 56-2. To a stirred solution of 6-(4-{1-[(tert-butyldimethylsilyl)oxy]ethenyl}pyrazol-1-yl)-N-(1-methylindazol-7-yl)pyridine-3-sulfonamide (90 mg, 0.176 mmol, 1 equiv.) in DCM (10 mL) were added ZnEt₂ (3.52 mL, 3.520 mmol, 20 equiv.) dropwise at 0° C. under a nitrogen atmosphere. The resulting mixture was stirred for an additional 30 min at 0° C. To the above mixture was added CH₂I₂(944.03 mg, 3.525 mmol, 20.00 equiv.) dropwise over 5 min at 0° C. The resulting mixture was stirred overnight at room temperature. The reaction was quenched by the addition of sat. NH₄Cl (aq.) (10 mL) and water/ice (10 mL) at 0° C. The resulting mixture was extracted with CH₂Cl₂ (3×5 mL). The combined organic layers were washed with brine (10 mL) and dried over anhydrous Na₂SO₄. After filtration, the filtrate was concentrated under reduced pressure to afford 6-(4-{1-[(tert-butyldimethylsilyl)oxy]cyclopropyl}pyrazol-1-yl)-N-(1-methylindazol-7-yl)pyridine-3-sulfonamide (80 mg, 86.51%) as a light yellow solid.

Step 3: Synthesis of 6-(4-(1-hydroxycyclopropyl)-1H-pyrazol-1-yl)-N-(1-methyl-1H-indazol-7-yl)pyridine-3-sulfonamide (I-56). To a stirred solution of 6-(4-{1-[(tert-butyldimethylsilyl)oxy]cyclopropyl}pyrazol-1-yl)-N-(1-methylindazol-7-yl)pyridine-3-sulfonamide (150 mg, 0.286 mmol, 1 equiv.) in DCM (10 mL) were added Et₃N·3HF (1382.55 mg, 8.580 mmol, 30 equiv.) dropwise at 0° C. under a nitrogen atmosphere. The resulting mixture was stirred for an additional 5 h at room temperature. The reaction was quenched by the addition of sat. NH₄Cl (aq.) and ice water (10 mL) at 0° C. The resulting mixture was extracted with CH₂Cl₂ (3×10 mL). The combined organic layers were washed with brine (10 mL) and dried over anhydrous Na₂SO₄. After filtration, the filtrate was concentrated under reduced pressure. The crude product was purified by prep-HPLC with the following conditions: column: XBridge Prep Phenyl OBD, 19*250 mm, 5 m; mobile phase A: water(10 mmol/L NH₄HCO₃+0.05% NH3H₂O), mobile phase B: ACN; flow rate 25 mL/min; gradient: 13% B to 32% B in 10 min; wavelength: 254 nm/220 nm; Rt1(min): 9.72; number of runs: 3) to afford 6-[4-(1-hydroxycyclopropyl)pyrazol-1-yl]-N-(1-methylindazol-7-yl)pyridine-3-sulfonamide (40 mg, 33.51%) as an off-white solid. LCMS: (ES, m/z): [M+H]⁺ 411.00; (400 MHz, DMSO-d₆) δ 0.89-0.92 (m, 2H), 1.04-1.07 (m, 2H), 4.29 (s, 3H), 6.07 (s, 1H), 6.52 (d, J7.2 Hz, 1H), 6.94 (t, J 7.6 Hz, 1H), 7.70 (d, J8.0 Hz, 1H), 7.76 (s, 1H), 8.06-8.10 (m, 2H), 8.19 (dd, J8.8, 2.4 Hz, 1H), 8.43 (s, 1H), 8.58 (d, J2.4 Hz, 1H), 10.28 (s, 1H).

### Example 185—Synthesis of 6-(6,7-Dihydropyrano[4,3-c]Pyrazol-1(4H)-yl)-N-(1-Methyl-1H-Indazol-7-yl)Pyridine-3-Sulfonamide (I-245)

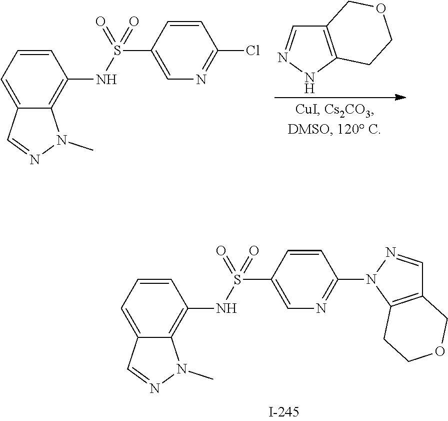

A solution of 6-chloro-N-(1-methylindazol-7-yl)pyridine-3-sulfonamide (300 mg, 0.929 mmol, 1 equiv.), 1H,4H,6H,7H-pyrano[4,3-c]pyrazole (288.46 mg, 2.323 mmol, 2.5 equiv.), CuI (708.06 mg, 3.716 mmol, 4 equiv.), Cs₂CO₃ (1817.00 mg, 5.574 mmol, 6 equiv.) and (1R,2R)—N1,N2-dimethylcyclohexane-1,2-diamine (264.41 mg, 1.858 mmol, 2 equiv.) in DMSO (10 mL) was stirred for 5 h at 120° C. under a nitrogen atmosphere. The reaction was quenched by the addition of water (10 mL) at 0° C. The aqueous layer was extracted with CH₂Cl₂ (2×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 50% to 65% gradient in 10 min; detector: UV 254 nm to afford N-(1-methylindazol-7-yl)-6-{4H,6H,7H-pyrano[4,3-c]pyrazol-1-yl}pyridine-3-sulfonamide (crude 230 mg) as a yellow solid. The residue was purified by prep-HPLC (Column: CHIRALPAKIG-34.6*50 mm, 3.0 um; mobile phase A: hex (0.2% DEA): (MeOH:DCM=1:1)=60:40; gradient: isocratic; injection volume: 2.0 mL) to afford N-(1-methylindazol-7-yl)-6-{4H,6H,7H-pyrano[4,3-c]pyrazol-1-yl}pyridine-3-sulfonamide (19.5 mg, 5.08%) as a yellow solid. LCMS: (ES, m/z): [M+H]⁺ 411; (400 MHz, DMSO-d₆) δ 3.22 (t, J5.2 Hz, 2H), 3.85 (t, J5.2 Hz, 2H), 4.28 (s, 3H), 4.64 (s, 2H), 6.53 (d, J7.2 Hz, 1H), 6.95 (t, J 7.6 Hz, 1H), 7.68-7.75 (m, 2H), 8.02-8.19 (m, 3H), 8.58 (d, J2.0 Hz, 1H), 10.27 (s, 1H).

### Example 186—Synthesis of Ethyl 5-Methyl-1-(5-(N-(1-Methyl-1H-Indazol-7-yl)Sulfamoyl)Pyridin-2-yl)-1H-Pyrazole-4-Carboxylate (I-246)

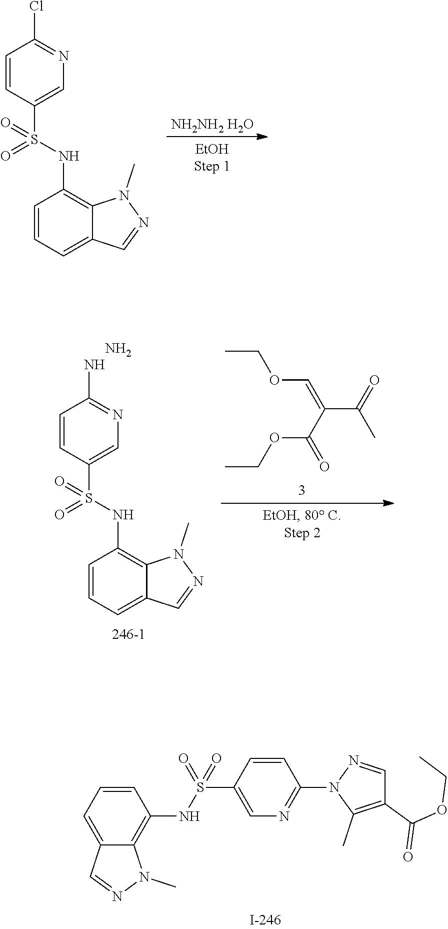

Step 1: Synthesis of 246-1. To a stirred mixture of 6-chloro-N-(1-methylindazol-7-yl)pyridine-3-sulfonamide (500 mg, 1.549 mmol, 1 equiv.) and hydrazine hydrate (80%) (775.49 mg, 15.490 mmol, 10 equiv.) in EtOH (6 mL) was added TEA (470.27 mg, 4.647 mmol, 3 equiv.) at room temperature under a nitrogen atmosphere. The resulting mixture was stirred overnight at 80° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. This resulted in 6-hydrazinyl-N-(1-methylindazol-7-yl)pyridine-3-sulfonamide (450 mg, 73.00%) as a light brown oil.

Step 2: Synthesis of ethyl 5-methyl-1-(5-(N-(1-methyl-1H-indazol-7-yl)sulfamoyl)pyridin-2-yl)-1H-pyrazole-4-carboxylate (1-246). A mixture of 6-hydrazinyl-N-(1-methylindazol-7-yl)pyridine-3-sulfonamide (50 mg, 0.157 mmol, 1 equiv.) and ethyl (2Z)-2-(ethoxymethylidene)-3-oxobutanoate (87.73 mg, 0.471 mmol, 3 equiv.) in EtOH (1 mL) was stirred for 2 h at 80° C. under a nitrogen atmosphere. The reaction was monitored by LCMS. The precipitated solids were collected by filtration and washed with EtOAc (2×1 mL). This resulted in ethyl 5-methyl-1-{5-[(1-methylindazol-7-yl)sulfamoyl]pyridin-2-yl}pyrazole-4-carboxylate (37.7 mg, 53.84%) as a pink solid. LCMS: (ES, m/z): [M+H]⁺ 441; ¹H NMR (400 MHz, DMSO-d₆) δ 1.31 (t, J7.1 Hz, 3H), 2.92 (s, 3H), 4.28 (q, J7.2 Hz, 2H), 4.30 (s, 3H), 6.54 (dd, J7.2, 1.2 Hz, 1H), 6.95 (t, J7.6 Hz, 1H), 7.72 (dd, J=8.0, 1.2 Hz, 1H), 8.11 (s, 1H), 8.12 (d, J8.8 Hz, 1H), 8.17 (s, 1H), 8.27 (dd, J=8.4, 2.4 Hz, 1H), 8.70 (d, J2.4 Hz, 1H), 10.40 (s, 1H).

### Example 187—Synthesis of 6-(Methyl((1R,2R)-2-(Methylamino) Cyclohexyl)Amino)-N-(1-Methyl-1H-Indazol-7-yl)Pyridine-3-Sulfonamide (I-248)

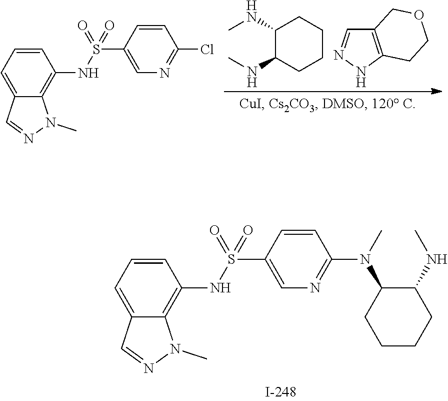

A solution of 6-chloro-N-(1-methylindazol-7-yl)pyridine-3-sulfonamide (300 mg, 0.929 mmol, 1 equiv.), 1H,4H,6H,7H-pyrano[4,3-c]pyrazole (288.46 mg, 2.323 mmol, 2.5 equiv.), CuI (708.06 mg, 3.716 mmol, 4 equiv.), Cs₂CO₃ (1817.00 mg, 5.574 mmol, 6 equiv.) and (1R,2R)—N1,N2-dimethylcyclohexane-1,2-diamine (264.41 mg, 1.858 mmol, 2 equiv.) in DMSO (10 mL) was stirred for 5 h at 120° C. under a nitrogen atmosphere. The reaction was quenched by the addition of water (10 mL) at 0° C. The aqueous layer was extracted with CH₂Cl₂ (2×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 45% to 60% gradient in 10 min; detector: UV 254 nm to afford 6-{methyl[(1R,2R)-2-(methylamino)cyclohexyl]amino}-N-(1-methylindazol-7-yl)pyridine-3-sulfonamide (crude 80 mg) as a yellow solid. The crude product (80 mg) was purified by prep-HPLC with the following conditions: column: Xselect CSH C18 OBD, 30*150 mm 5 m; mobile phase A: water (0.1% FA), mobile phase B: ACN; flow rate 60 mL/min; gradient: 12% B to 25% B in 7 min; wavelength: 254 nm/220 nm; Rt1(min): 6.08) to afford 6-{methyl[(1R,2R)-2-(methylamino)cyclohexyl]amino}-N-(1-methylindazol-7-yl)pyridine-3-sulfonamide (44.5 mg, 11.07%) as a yellow solid. LCMS: (ES, m/z): [M+H]⁺ 429; (400 MHz, DMSO-d₆) δ 1.21-1.39 (m, 3H), 1.58-1.61 (m, 2H), 1.71-1.73 (m, 2H), 2.08-2.13 (m, 1H), 2.40 (s, 3H), 2.87 (s, 3H), 3.16 (s, 1H), 4.32 (s, 3H), 4.74 (s, 1H), 6.64 (d, J7.2 Hz, 1H), 6.76-6.86 (m, 2H), 7.41 (d, J8.0 Hz, 1H), 7.76 (dd, J9.2, 2.4 Hz, 1H), 7.96 (s, 1H), 8.25 (d, J2.4 Hz, 2H).

### Example 188—Synthesis of N-(3-Fluoro-6-Methoxy-1-Methyl-1H-Indazol-7-yl)-6-(4-Methyl-1H-Pyrazol-1-yl)Pyridine-3-Sulfonamide (I-249)

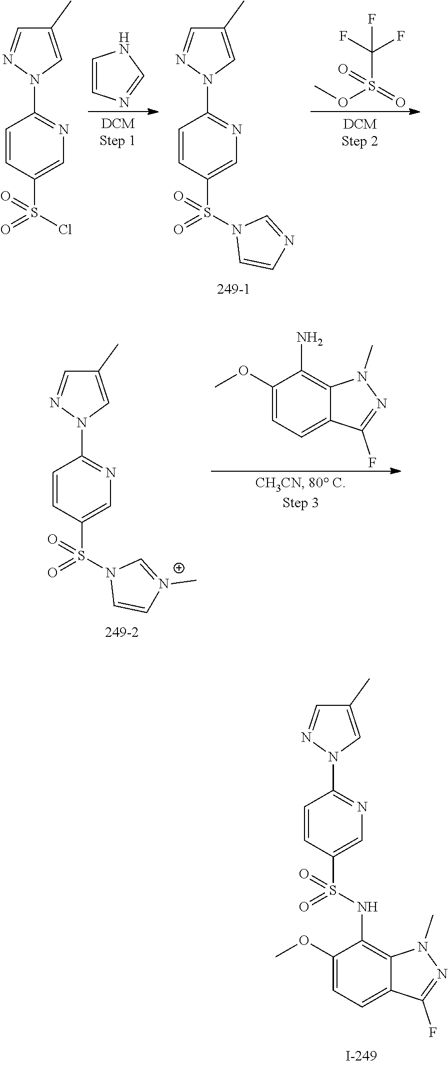

Step 1: Synthesis of 249-1. A solution of 6-(4-methylpyrazol-1-yl)pyridine-3-sulfonyl chloride (330 mg, 1.281 mmol, 1 equiv.) and imidazole (131 mg, 1.924 mmol, 1.50 equiv.) in DCM (8 mL) was stirred for 3 h at 0° C. under a nitrogen atmosphere. The resulting mixture was diluted with water (10 mL). The resulting mixture was extracted with CH₂Cl₂ (3×15 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm to afford 5-(imidazole-1-sulfonyl)-2-(4-methylpyrazol-1-yl)pyridine (300 mg, 75.31%) as a light yellow solid.

Step 2: Synthesis of 249-2. A solution of 5-(imidazole-1-sulfonyl)-2-(4-methylpyrazol-1-yl)pyridine (300 mg, 1.037 mmol, 1 equiv.) and triflate ester (340 mg, 2.072 mmol, 2.00 equiv.) in DCM (10 mL) was stirred for 3 h at 0° C. under a nitrogen atmosphere. The reaction was quenched with water (10 mL) and then extracted with DCM (3×10 mL). The combined organic layers were dried over anhydrous Na₂SO₄. After filtration, the filtrate was concentrated under reduced pressure. The crude product was used in the next step directly without further purification.

Step 3: Synthesis of N-(3-fluoro-6-methoxy-1-methyl-1H-indazol-7-yl)-6-(4-methyl-1H-pyrazol-1-yl)pyridine-3-sulfonamide (1-249). A mixture of 1-methyl-3-[6-(4-methylpyrazol-1-yl)pyridin-3-ylsulfonyl]imidazol-1-ium triflate (260 mg, 0.573 mmol, 1 equiv.) and 3-fluoro-6-methoxy-1-methylindazol-7-amine (56 mg, 0.287 mmol, 0.50 equiv.) in MeCN (10 mL) was stirred overnight at 80° C. under a nitrogen atmosphere. The resulting mixture was concentrated under reduced pressure. The resulting mixture was diluted with water (15 mL). The resulting mixture was extracted with EtOAc (3×20 mL). The combined organic layers were washed with brine (10 mL) and dried over anhydrous Na₂SO₄. After filtration, the filtrate was concentrated under reduced pressure. The residue was purified by prep-TLC (CH₂Cl₂/MeOH 20:1). The residue was washed with MeCN (3×1 mL) to afford N-(3-fluoro-6-methoxy-1-methylindazol-7-yl)-6-(4-methylpyrazol-1-yl)pyridine-3-sulfonamide (34.6 mg, 14.42%) as a pink solid. LCMS: (ES, m/z): [M+H]⁺ 417; (400 MHz, DMSO-d₆) 2.13 (s, 3H), 3.41 (s, 3H), 4.28 (s, 3H), 7.64 (d, J 10.8 Hz, 1H), 7.78 (s, 1H), 8.04-8.06 (m, 2H), 8.25 (dd, J8.8, 2.4 Hz, 1H), 8.49 (s, 1H), 8.68 (d, J2.4 Hz, 1H), 10.44 (s, 1H).

### Example 189—Synthesis of 6-(2-Chlorothiazol-5-yl)-N-(1-Methyl-1H-Indazol-7-yl)Pyridine-3-Sulfonamide (I-250)

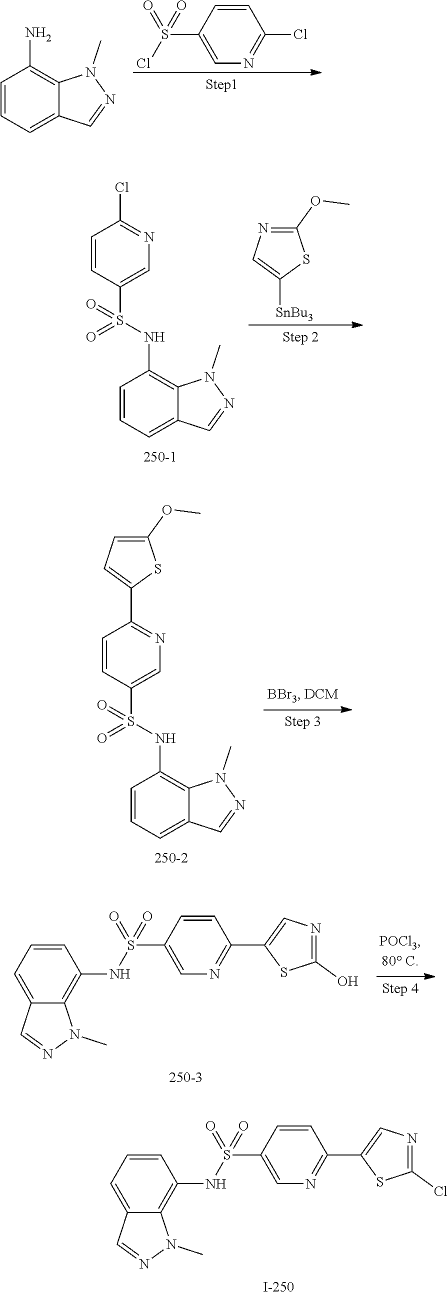

Step 1: Synthesis of 250-1. To a solution of 1-methylindazol-7-amine (20 g, 135.887 mmol, 1 equiv.) in pyridine (500 mL) was added 6-chloropyridine-3-sulfonyl chloride (34.58 g, 163.064 mmol, 1.2 equiv.) in portions over 10 min at room temperature. The resulting mixture was stirred for an additional 3 h at room temperature. The resulting mixture was concentrated under reduced pressure. The residue was purified by silica gel column chromatography, eluted with PE/EA (5:1) to afford 6-chloro-N-(1-methylindazol-7-yl)pyridine-3-sulfonamide (39.7 g, 90.51%) as a yellow solid.

Step 2: Synthesis of 250-2. A mixture of 6-chloro-N-(1-methylindazol-7-yl)pyridine-3-sulfonamide (472 mg, 1.462 mmol, 1 equiv.), 2-methoxy-5-(tributylstannyl)-1,3-thiazole (709.29 mg, 1.754 mmol, 1.2 equiv.), XPhos (139.43 mg, 0.292 mmol, 0.2 equiv.) and XPhos Pd G3 (247.56 mg, 0.292 mmol, 0.2 equiv.) in dioxane (2 mL) was stirred for 2 h at 90° C. under a nitrogen atmosphere. The reaction was quenched by the addition of water (20 mL) at room temperature. The residue was purified by reversed-phase flash chromatography with the following conditions: column: C18 silica gel; mobile phase: MeCN in water (10 mmol/L NH₄HCO₃), 30% to 50% gradient in 10 min; detector: UV 254 nm. This resulted in N-(1H-indazol-7-yl)-6-(2-methoxy-1,3-thiazol-5-yl)pyridine-3-sulfonamide (480 mg, 81.76%) as an off-white solid.

Step 3: Synthesis of 250-3. A solution of N-(1H-indazol-7-yl)-6-(2-methoxy-1,3-thiazol-5-yl)pyridine-3-sulfonamide (300 mg, 0.774 mmol, 1 equiv.) in DCM (3 mL) was stirred for 20 min at 0° C. under a nitrogen atmosphere followed by the addition of BBr₃ (581.96 mg, 2.322 mmol, 3 equiv.) dropwise at 0° C. The resulting mixture was stirred overnight at room temperature. The reaction was quenched by the addition of water/ice (30 mL) at room temperature. The resulting mixture was concentrated under reduced pressure. The residue was purified by reversed-phase flash chromatography with the following conditions: column: C18 silica gel; mobile phase: MeCN in water (10 mmol/L NH₄HCO₃), 30% to 50% gradient in 10 min; detector: UV 254 nm. This resulted in 6-(2-hydroxy-1,3-thiazol-5-yl)-N-(1-methylindazol-7-yl)pyridine-3-sulfonamide (160 mg, 53.33%) as a white solid.

Step 4: Synthesis of 6-(2-chlorothiazol-5-yl)-N-(1-methyl-1H-indazol-7-yl)pyridine-3-sulfonamide (I-250). A solution of 6-(2-hydroxy-1,3-thiazol-5-yl)-N-(1-methylindazol-7-yl)pyridine-3-sulfonamide (160 mg, 0.413 mmol, 1 equiv.) in phosphorus oxychloride (20 mL, 0.130 mmol) was stirred overnight at 80° C. under a nitrogen atmosphere. The resulting mixture was concentrated under reduced pressure. The reaction was quenched with water (2 mL) carefully at 0° C. The residue was purified by reversed-phase flash chromatography with the following conditions: column: C18 silica gel; mobile phase: MeCN in water (10 mmol/L NH₄HCO₃), 30% to 50% gradient in 10 min; detector: UV 254 nm. This resulted in 6-(2-chloro-1,3-thiazol-5-yl)-N-(1-methylindazol-7-yl)pyridine-3-sulfonamide (15 mg, 8.82%) as a white solid. (ES, m/z): [M+H]⁺406; ¹H NMR (400 MHz, DMSO-d₆) δ 4.29 (s, 3H), 6.53 (d, J7.2 Hz, 1H), 6.91 (t, J7.6 Hz, 1H), 7.62 (d, J6.0 Hz, 1H), 8.07 (s, 1H), 8.13 (d, J8.4, 2.4 Hz, 1H), 8.25 (d, J8.4 Hz, 1H), 8.58 (s, 1H), 8.75 (d, J 1.6 Hz, 1H), 10.36 (s, 1H).

### Example 190—Synthesis of 6-(4-(1-Methoxycyclopropyl)-1H-Pyrazol-1-yl)-N-(1-Methyl-1H-Indazol-7-yl)Pyridine-3-Sulfonamide (I-251)

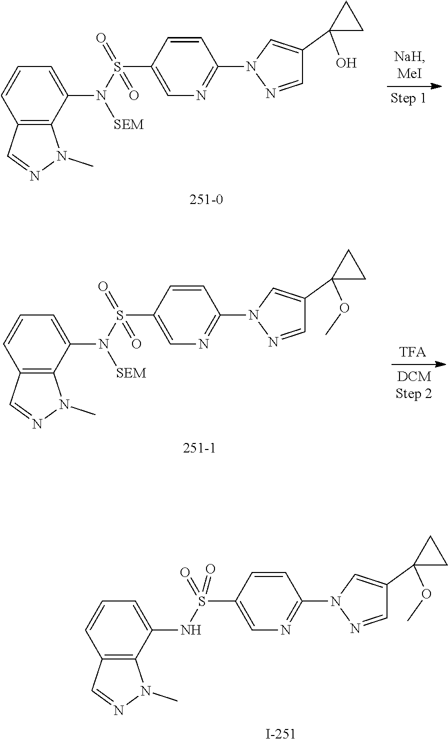 [SMILES: Cn1ncc2cccc(N(COCC[Si](C)(C)C)S(=O)(=O)c3ccc(-n4cc(C5(O)CC5)cn4)nc3)c21 ; COC1(c2cnn(-c3ccc(S(=O)(=O)N(COCC[Si](C)(C)C)c4cccc5cnn(C)c45)cn3)c2)CC1 ; COC1(c2cnn(-c3ccc(S(=O)(=O)Nc4cccc5cnn(C)c45)cn3)c2)CC1]

Step 1: Synthesis of 251-1. To a stirred solution of 6-[4-(1-hydroxycyclopropyl)pyrazol-1-yl]-N-(1-methylindazol-7-yl)-N-{[2-(trimethylsilyl)ethoxy]methyl}pyridine-3-sulfonamide (120 mg, 0.222 mmol, 1 equiv.) in THE (10 mL) were added NaH (17.75 mg, 0.444 mmol, 2.0 equiv., 60%) in portions at 0° C. under a nitrogen atmosphere. The resulting mixture was stirred for an additional 30 min at 0° C. To the above mixture was added Mel (63.00 mg, 0.444 mmol, 2.0 equiv.) dropwise over 5 min at 0° C. The resulting mixture was stirred for an additional 5 h at room temperature. The reaction was quenched by the addition of sat. NH₄Cl (aq.) (5 mL) and water/ice (5 mL) at 0° C. The resulting mixture was extracted with CH₂Cl₂ (3×5 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₄HCO₃), 30% to 70% gradient in 10 min; detector: UV 220 nm to afford 6-[4-(1-methoxycyclopropyl)pyrazol-1-yl]-N-(1-methylindazol-7-yl)-N-{[2-(trimethylsilyl)ethoxy]methyl}pyridine-3-sulfonamide (80 mg, 64.98%) as a light yellow solid.

Step 2: Synthesis of 6-(4-(1-methoxycyclopropyl)-1H-pyrazol-1-yl)-N-(1-methyl-1H-indazol-7-yl)pyridine-3-sulfonamide (1-251). To a stirred solution of 6-[4-(1-methoxycyclopropyl)pyrazol-1-yl]-N-(1-methylindazol-7-yl)-N-{[2-(trimethylsilyl)ethoxy]methyl}pyridine-3-sulfonamide (90 mg, 0.162 mmol, 1 equiv.) in DCM (10 mL) was added TFA (1 mL, 13.463 mmol, 82.98 equiv.) dropwise at 0° C. under a nitrogen atmosphere. The resulting mixture was stirred for an additional 5 h at room temperature. The resulting mixture was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 220 nm to afford crude product as a yellow solid. The crude product was purified by prep-HPLC with the following conditions: column: XBridge Prep Phenyl OBD, 19*250 mm, 5 m; mobile phase A: water(0.05% TFA), mobile phase B: ACN; flow rate 25 mL/min; gradient: 43% B to 54% B in 9 min; wavelength: 254 nm/220 nm; Rt1(min): 8.32, 8.8(min):) to afford 6-[4-(1-methoxycyclopropyl)pyrazol-1-yl]-N-(1-methylindazol-7-yl)pyridine-3-sulfonamide (50 mg, 72.53%) as an off-white solid. LCMS: (ES, m/z): [M+H]⁺ 424.95; (400 MHz, DMSO-d₆) δ 0.94-0.97 (m, 2H), 1.10-1.13 (m, 2H), 3.22 (s, 3H), 4.29 (s, 3H), 6.51 (d, J7.2 Hz, 1H), 6.94 (t, J 7.6 Hz, 1H), 7.71 (d, J7.6 Hz, 1H), 7.89 (s, 1H), 8.09-8.12 (m, 2H), 8.22 (dd, J8.8, 2.4 Hz, 1H), 8.55-8.59 (m, 2H), 10.30 (s, 1H).

### Example 191—Synthesis of 6-(4-(Hydroxymethyl)-5-Methyl-1H-Pyrazol-1-yl)-N-(1-Methyl-1H-Indazol-7-yl)Pyridine-3-Sulfonamide (I-252)

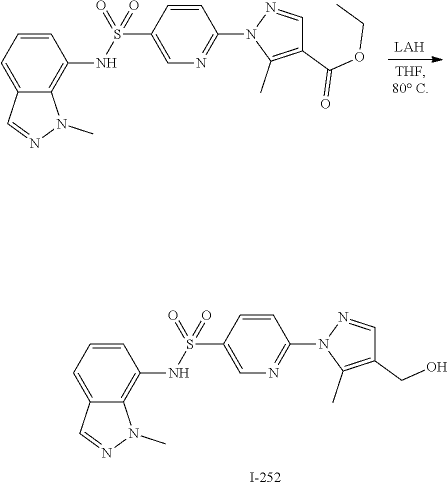 [SMILES: CCOC(=O)c1cnn(-c2ccc(S(=O)(=O)Nc3cccc4cnn(C)c34)cn2)c1C ; Cc1c(CO)cnn1-c1ccc(S(=O)(=O)Nc2cccc3cnn(C)c23)cn1]

A mixture of ethyl 5-methyl-1-{5-[(1-methylindazol-7-yl)sulfamoyl]pyridin-2-yl}pyrazole-4-carboxylate (100 mg, 0.227 mmol, 1 equiv.) and LiAlH₄ (1.72 mg, 0.045 mmol, 0.2 equiv.) in THF (3 mL) was stirred for 2 h at 80° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. This resulted in 6-[4-(hydroxymethyl)-5-methylpyrazol-1-yl]-N-(1-methylindazol-7-yl)pyridine-3-sulfonamide (17.3 mg, 19.11%) as an off-white solid. LCMS (ES, m/z): [M+H]⁺ 399; ¹H NMR (400 MHz, DMSO-d₆) δ 2.62 (s, 3H), 4.29 (s, 3H), 4.39 (d, J 5.2 Hz, 2H), 4.93 (t, J5.2 Hz, 1H), 6.53 (d, J7.2 Hz, 1H), 6.95 (t, J7.6 Hz, 1H), 7.70 (d, J8.0 Hz, 1H), 7.76 (s, 1H), 8.08 (d, J 9.2 Hz, 1H), 8.09 (s, 1H).8.18 (dd, J=8.8, 2.4 Hz, 1H), 8.61 (d, J2.4 Hz, 1H), 10.29 (s, 1H).

### Example 192—Synthesis of N-(1-Methyl-1H-Indazol-7-yl)-6-(1-(Oxetan-3-yl)-1H-Pyrazol-4-yl)Pyridine-3-Sulfonamide (I-253)

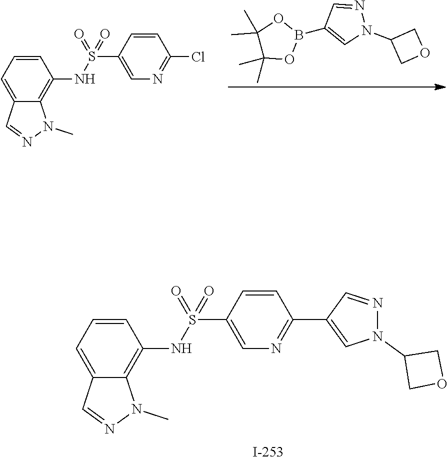 [SMILES: Cn1ncc2cccc(NS(=O)(=O)c3ccc(Cl)nc3)c21 ; CC1(C)OB(c2cnn(C3COC3)c2)OC1(C)C ; Cn1ncc2cccc(NS(=O)(=O)c3ccc(-c4cnn(C5COC5)c4)nc3)c21]

A solution of N-(6-methoxy-1-methylindazol-7-yl)-1H-pyrazole-4-sulfonamide (100 mg, 0.325 mmol, 1 equiv.) in dioxane (3 mL) was treated with 2-(2-bromopyridin-4-yl)propan-2-ol (84.37 mg, 0.390 mmol, 1.2 equiv.) for 2 min at room temperature under a nitrogen atmosphere followed by the addition of EPhos (34.80 mg, 0.065 mmol, 0.2 equiv.), EPhos Pd G4 (59.78 mg, 0.065 mmol, 0.2 equiv.) and Cs₂CO₃ (318.05 mg, 0.975 mmol, 3 equiv.) in portions. The resulting mixture was stirred for an additional 2 h at 90° C. The resulting mixture was concentrated under reduced pressure. The residue was purified by reversed-phase flash chromatography with the following conditions: column: C18 silica gel; mobile phase: MeCN in water (0.1% FA), 0% to 50% gradient in 20 min; detector: UV 254 nm. This resulted in N-(1-methyl-1H-indazol-7-yl)-6-(1-(oxetan-3-yl)-1H-pyrazol-4-yl)pyridine-3-sulfonamide (20.0 mg, 24.00%) as a white solid. LCMS: (ES, m/z): [M+H]⁺411; (400 MHz, DMSO-d₆): δ 4.27 (s, 3H), 4.91-4.98 (m, 4H), 5.62-5.67 (m, 1H), 6.49 (d, J6.8 Hz, 1H), 6.92 (t, J8.0 Hz, 1H), 7.68 (d, J7.6 Hz, 1H), 7.91 (d, J8.4 Hz, 1H), 7.99 (dd, J8.4, 2.4 Hz, 1H), 8.09 (s, 1H), 8.28 (s, 1H), 8.66-8.68 (m, 2H), 10.20 (s, 1H).

### Example 193—Synthesis of N-(1,6-Dimethyl-1H-Indazol-7-yl)-6-(5-(Trifluoromethyl)-1H-Pyrazol-3-yl)Pyridine-3-Sulfonamide (I-63)

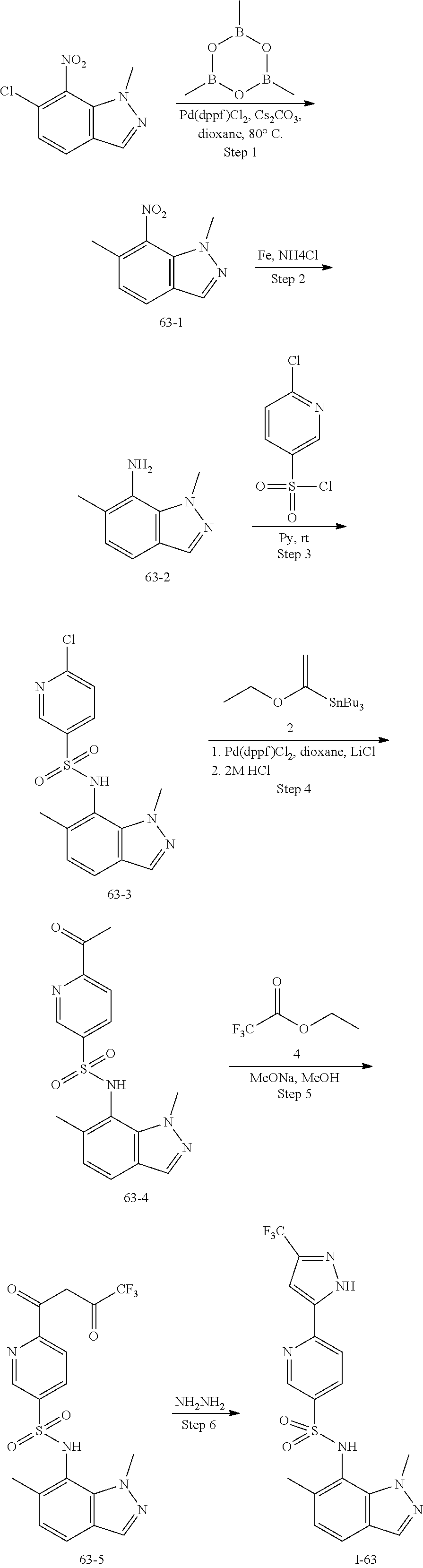

Step 1: Synthesis of 63-1. A solution of 6-chloro-1-methyl-7-nitroindazole (1000 mg, 4.73 mmol, 1 equiv.), trimethyl-1,3,5,2,4,6-trioxatriborinane (893 mg, 7.095 mmol, 1.5 equiv.), Pd(dppf)Cl₂ (721.9 mg, 0.94 mmol, 0.2 equiv.) and Cs₂CO₃ (3102 mg, 9.46 mmol, 2 equiv.) in 1,4-dioxane (10 mL) was stirred overnight at 80° C. under a nitrogen atmosphere. The resulting mixture was diluted with water (50 mL). The resulting mixture was extracted with EtOAc (3×50 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (5 mmol/L NH₄HCO₃), 0% to 100% gradient in 20 min; detector: UV 254 nm, to give 1,6-dimethyl-7-nitroindazole (800 mg, 81.46%) as a yellow solid.

Step 2: Synthesis of 63-2. A solution of 1,6-dimethyl-7-nitroindazole (800 mg, 4.1 mmol, 1 equiv.), Fe (2296 mg, 41 mmol, 10 equiv.) and NH₄Cl (1158.25 mg, 20.5 mmol, 5 equiv.) in EtOH (20 mL) and H₂O (10 mL) was stirred for 5 h at room temperature. The mixture was filtrated and the filtrate was concentrated under reduced pressure. The resulting mixture was diluted with water (20 mL) and extracted with EtOAc (3×20 mL). The combined organic layers were washed with brine (50 mL) and dried over anhydrous Na₂SO₄. After filtration, the filtrate was concentrated under reduced pressure to give 1,6-dimethylindazol-7-amine (641 mg, 76.85%) as a brown solid.

Step 3: Synthesis of 63-3. A solution of 1,6-dimethylindazol-7-amine (641 mg, 3.981 mmol, 1 equiv.) and 6-chloropyridine-3-sulfonyl chloride (1266.1 mg, 5.972 mmol, 1.5 equiv.) in pyridine (5 mL) was stirred overnight at room temperature. The mixture was concentrated under reduced pressure. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (5 mmol/L NH₄HCO₃), 0% to 100% gradient in 20 min; detector: UV 254 nm, to give 6-chloro-N-(1,6-dimethylindazol-7-yl)pyridine-3-sulfonamide (322 mg, 22.74%) as purple solid.

Step 4: Synthesis of 63-4. A solution of 6-chloro-N-(1,6-dimethylindazol-7-yl)pyridine-3-sulfonamide (311 mg, 0.923 mmol, 1 equiv.), Pd(dppf)Cl₂ (135.14 mg, 0.185 mmol, 0.2 equiv.), LiCl (78.29 mg, 1.846 mmol, 2 equiv.) and tributyl(1-ethoxyethenyl)stannane (667.00 mg, 1.846 mmol, 2 equiv.) in 1,4-dioxane (10 mL) was stirred for 3 h at 100° C. under a nitrogen atmosphere. The mixture was treated with 2 M HCl (2 mL) for 30 min. The reaction was quenched by the addition of KF (aq.) (20 mL) at room temperature. The aqueous layer was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give 6-acetyl-N-(1,6-dimethylindazol-7-yl)pyridine-3-sulfonamide (306 mg, 94.30%) as a yellow solid.

Step 5: Synthesis of 63-5. A solution of 6-acetyl-N-(1,6-dimethylindazol-7-yl)pyridine-3-sulfonamide (160 mg, 0.465 mmol, 1 equiv.), MeONa (250.99 mg, 4.650 mmol, 10 equiv.) and trifluoroethyl acetate (132.01 mg, 0.930 mmol, 2 equiv.) in MeOH (5 mL) was stirred for 3 h at 70° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give N-(1,6-dimethylindazol-7-yl)-6-(4,4,4-trifluoro-3-oxobutanoyl)pyridine-3-sulfonamide (150 mg, 73.31%) as a yellow solid.

Step 6: N-(1,6-dimethyl-1H-indazol-7-yl)-6-(5-(trifluoromethyl)-1H-pyrazol-3-yl)pyridine-3-sulfonamide (I-63). A solution of N-(1,6-dimethylindazol-7-yl)-6-(4,4,4-trifluoro-3-oxobutanoyl)pyridine-3-sulfonamide (150 mg, 0.341 mmol, 1 equiv.) and NH₂NH₂·H₂O (85.25 mg, 1.705 mmol, 5 equiv.) in EtOH (4 mL) was stirred overnight at 50° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by prep-HPLC with the following conditions: column: XBridge Prep OBD C18, 30*150 mm, 5 m; mobile phase A: water(10 mmol/L NH₄HCO₃), mobile phase B: ACN; flow rate 60 mL/min; gradient: 26% B to 46% B in 8 min, 46% B; wavelength: 254 nm; Rt1(min): 7.95; injection volume: 0.7 mL) to afford N-(1,6-dimethylindazol-7-yl)-6-[5-(trifluoromethyl)-1H-pyrazol-3-yl]pyridine-3-sulfonamide (10.3 mg, 6.92%) as a white solid. LCMS: (ES, m/z): [M+H]⁺ 437; (400 MHz, DMSO-d₆) δ 1.65 (s, 3H), 4.25 (s, 3H), 6.86(d, J 8.4 Hz, 1H), 7.56 (d, J8.8 Hz, 2H), 7.99 (s, 1H) 8.13 (dd, J8.0 Hz, 2.0 Hz, 2H), 8.75 (d, J 1.6 Hz, 1H), 10.33 (s, 1H), 14.64 (s, 1H).

### Example 194—Synthesis of N-(6-Methoxy-1-Methyl-1H-Pyrazolo[4,3-C]Pyridin-7-yl)-6-(4-(Trifluoromethyl)-1H-Pyrazol-1-yl)Pyridine-3-Sulfonamide (I-74)

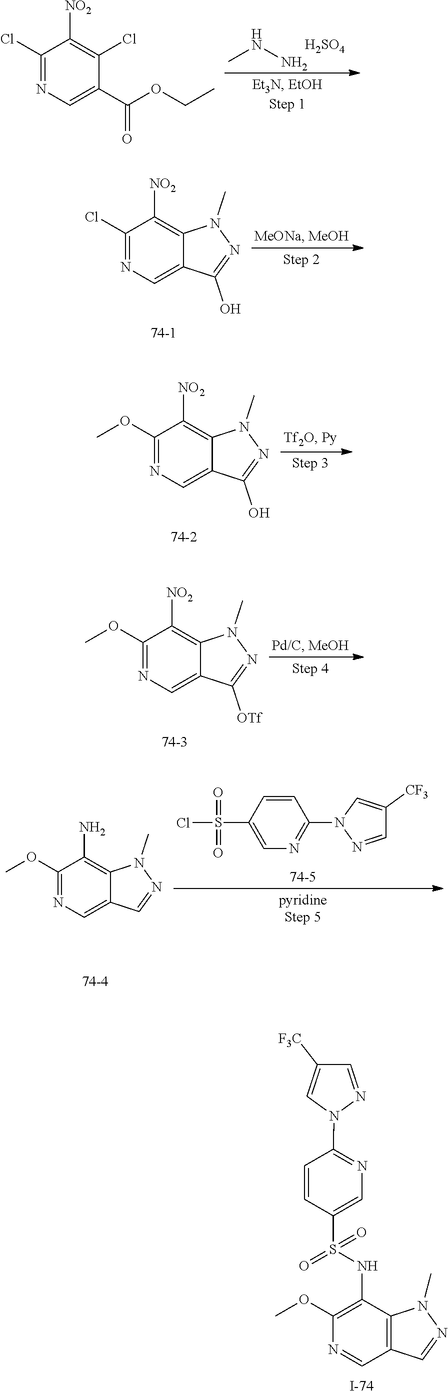

Step 1: Synthesis of 74-1. A solution of ethyl 4,6-dichloro-5-nitropyridine-3-carboxylate (1.56 g, 3.99 mmol, 1 equiv.), Et₃N (1.212 g, 11.97 mmol, 3 equiv.) and methyl hydrazine sulfuric acid salt (633.6 mg, 4.39 mmol, 1.1 equiv.) in EtOH (20 mL) was stirred overnight at room temperature. After the reaction was completed, the solution was concentrated under vacuum. The crude was purified by reversed-phase flash chromatography with the following conditions: column: C18 silica gel; mobile phase: ACN in water, 0% to 100% gradient in 20 min; detector: UV 254 nm to give 74-1 (627 mg, 68.92%) as a red solid.

Step 2: Synthesis of 74-2. A solution of 74-1 (627 mg, 2.73 mmol, 1 equiv.) and CH₃ONa (738.2 mg, 13.67 mmol, 10 equiv.) in CH₃OH (20 mL) was stirred overnight at 80° C. After the reaction was completed, the mixture was concentrated and purified by reversed-phase flash chromatography with the following conditions: column: C18 silica gel; mobile phase: ACN in water, 0% to 100% gradient in 15 min; detector: UV 254 nm to give 74-2 (475 mg, 77.7%) as a brown solid.

Step 3: Synthesis of 74-3. A solution of 74-2 (475 mg, 2.11 mmol, 1 equiv.) in DCM (20 mL) was treated with pyridine (667.1 mg,8.44 mmol, 4.00 equiv.) at room temperature followed by the addition of Tf₂O (1487.6 mg, 5.28 mmol, 2.5 equiv.) dropwise at 0° C. This reaction was stirred overnight from 0° C. to room temperature. After the reaction was completed, the resulting mixture was diluted with water (50 mL). The resulting mixture was extracted with EtOAc (4×40 mL). The combined organic layers were washed with brine (50 mL) and dried over anhydrous Na₂SO₄. After filtration, the filtrate was concentrated under reduced pressure to give the crude and the crude was purified by prep-TLC (PE/EA 6.5:1) to afford 74-3 (594 mg, 47.20%) as a reddish semi-solid.

Step 4: Synthesis of 74-4. A solution of 74-3 (594 mg, 1.667 mmol, 1 equiv.) and Pd/C (100 mg, 0.940 mmol, 0.56 equiv.) in MeOH (15 mL) was stirred overnight at room temperature under a hydrogen atmosphere. After the reaction was completed, the solid was filtered. The filtrate was concentrated under vacuum to give 74-4 (522 mg) as a brown solid.

Step 5: Synthesis of N-(6-methoxy-1-methyl-1H-pyrazolo[4,3-c]pyridin-7-yl)-6-(4-(trifluoromethyl)-1H-pyrazol-1-yl)pyridine-3-sulfonamide (1-74). A solution of 74-4 (106.8 mg, 0.60 mmol, 1 equiv.) and 6-[4-(trifluoromethyl)pyrazol-1-yl]pyridine-3-sulfonyl chloride (186.79 mg, 0.60 mmol, 1 equiv.) in pyridine (6 mL) was stirred overnight at room temperature. After the reaction was completed, the mixture was concentrated and purified by reversed-phase flash chromatography with the following conditions: column: C18 silica gel; mobile phase: ACN in water, 0% to 100% gradient in 20 min; detector: UV 254 nm to give the crude. The residue was purified by prep-HPLC(Column: XBridge Prep OBD Cis, 30*150 mm, 5 m; mobile phase A: water(10 mmol/L NH₄HCO₃), mobile phase B: ACN; flow rate 60 mL/min; gradient: 5% B to 30% B in 8 min; wavelength: 254 nm; Rt1(min): 7.77; injection volume: 1 mL; number of runs: 3) to afford N-(6-methoxy-1-methyl-1H-pyrazolo[4,3-c]pyridin-7-yl)-6-(4-(trifluoromethyl)-1H-pyrazol-1-yl)pyridine-3-sulfonamide (6.6 mg, 2.42%) as a white solid. LCMS: (ES, m/z): [M+H]⁺434; (400 MHz, DMSO-d₆) δ 3.28 (s, 3H), 4.25 (s, 3H), 8.17 (d, J8.8 Hz, 1H), 8.22 (s, 1H), 8.29 (dd, J8.8, 2.4 Hz, 1H), 8.41 (s, 1H), 8.63 (s, 1H), 8.69 (d, J2.0 Hz, 1H), 9.33 (s, 1H), 10.27 (s, 1H).

### Example 195—Synthesis of N-(6-Ethyl-1-Methyl-1H-Indazol-7-yl)-6-(5-(Trifluoromethyl)-1H-Pyrazol-1-yl)Pyridine-3-Sulfonamide (I-126)

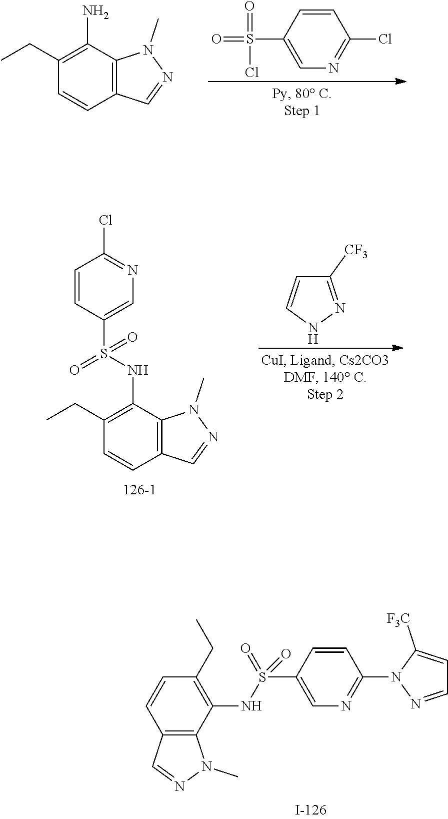 [SMILES: CCc1ccc2cnn(C)c2c1N ; O=S(=O)(Cl)c1ccc(Cl)nc1 ; CCc1ccc2cnn(C)c2c1NS(=O)(=O)c1ccc(Cl)nc1 ; FC(F)(F)c1cc[nH]n1 ; CCc1ccc2cnn(C)c2c1NS(=O)(=O)c1ccc(-n2nccc2C(F)(F)F)nc1]

Step 1: Synthesis of 126-1. A solution of 6-ethyl-1-methylindazol-7-amine (150 mg, 0.856 mmol, 1 equiv.) and 6-chloropyridine-3-sulfonyl chloride (272.26 mg, 1.284 mmol, 1.5 equiv.) in pyridine (2 mL) was stirred for 16 h at 80° C. under an air atmosphere. The mixture was concentrated under reduced pressure. The resulting mixture was diluted with water (10 mL) and extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 6-chloro-N-(6-ethyl-1-methylindazol-7-yl)pyridine-3-sulfonamide (90 mg, 29.07%) as a yellow solid.

Step 2: Synthesis of N-(6-ethyl-1-methyl-1H-indazol-7-yl)-6-(5-(trifluoromethyl)-1H-pyrazol-1-yl)pyridine-3-sulfonamide (I-126). A solution of 6-chloro-N-(6-ethyl-1-methylindazol-7-yl)pyridine-3-sulfonamide (60 mg, 0.171 mmol, 1 equiv.), 3-(trifluoromethyl)-1H-pyrazole (46.55 mg, 0.342 mmol, 2 equiv.), N1,N2-dimethylcyclohexane-1,2-diamine (24.33 mg, 0.171 mmol, 1 equiv.), CuI (16.29 mg, 0.086 mmol, 0.5 equiv.) and Cs₂CO₃ (167.17 mg, 0.513 mmol, 3 equiv.) in DMF (2 mL) was stirred for 16 h at 140° C. under a nitrogen atmosphere. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in N-(6-ethyl-1-methylindazol-7-yl)-6-[5-(trifluoromethyl)pyrazol-1-yl]pyridine-3-sulfonamide (22.8 mg, 29.45%) as a white solid. LCMS: (ES, m/z): [M+H]⁺ 451; (400 MHz, DMSO-d₆) δ 0.81 (t, J7.6 Hz, 3H), 2.06 (q, J7.6 Hz, 2H), 4.28 (s, 3H), 6.97 (d, J 8.4 Hz, 1H), 7.16 (d, J2.8 Hz, 1H), 7.67 (d, J 8.4 Hz, 1H), 8.03 (s, 1H), 8.21 (d, J8.8 Hz, 1H), 8.29 (dd, J8.8, 2.4 Hz, 1H), 8.69 (d, J2.4 Hz, 1H), 8.94 (dd, J2.8, 1.2 Hz, 1H),10.41 (s, 1H).

### Example 196—Synthesis of 6-(3-(Difluoromethyl)-1H-Pyrazol-1-yl)-N-(6-Methoxy-1-Methyl-1H-Indazol-7-yl)Pyridine-3-Sulfonamide (I-127)

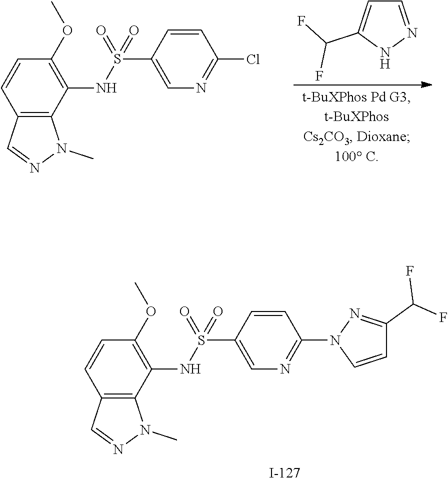 [SMILES: COc1ccc2cnn(C)c2c1NS(=O)(=O)c1ccc(Cl)nc1 ; FC(F)c1ccn[nH]1 ; COc1ccc2cnn(C)c2c1NS(=O)(=O)c1ccc(-n2ccc(C(F)F)n2)nc1]

To a stirred mixture of 6-chloro-N-(6-methoxy-1-methylindazol-7-yl)pyridine-3-sulfonamide (200 mg, 0.567 mmol, 1 equiv.), Cs₂CO₃ (554.13 mg, 1.701 mmol, 3 equiv.) and 3-(difluoromethyl)-2H-pyrazole (133.89 mg, 1.134 mmol, 2 equiv.) in dioxane (6 mL) were added t-BuXPhos Pd G3 (90.07 mg, 0.113 mmol, 0.2 equiv.) and t-BuXPhos (48.15 mg, 0.113 mmol, 0.2 equiv.) at room temperature under a nitrogen atmosphere. The resulting mixture was stirred overnight at 100° C. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by prep-HPLC with the following conditions (gradient: isocratic) to afford 6-[5-(difluoromethyl)pyrazol-1-yl]-N-(6-methoxy-1-methylindazol-7-yl)pyridine-3-sulfonamide (73.1 mg, 29.62%) as an off-white solid. LCMS: (ES, m/z): [M+H]⁺ 435; ¹H NMR (400 MHz, DMSO-d₆) δ 3.19 (s, 3H), 4.28 (s, 3H), 6.83 (d, J 8.8 Hz, 1H), 6.93 (d, J 2.8 Hz, 1H), 7.20 (d, J 54.4 Hz, 1H), 7.69 (d, J 8.8 Hz, 1H), 8.00 (s, 1H), 8.13 (d, J 8.8 Hz, 1H), 8.26 (dd, J 8.8, 2.4 Hz, 1H), 8.68 (d, J 2.4 Hz, 1H), 8.84 (d, J 2.8 Hz, 1H), 10.10 (s, 1H).

### Example 197—Synthesis of N-(6-Methoxy-1-Methyl-1H-Benzo[D][1,2,3]Triazol-7-yl)-6-(3-(Trifluoromethyl)-1H-Pyrazol-1-yl)Pyridine-3-Sulfonamide (1-128)

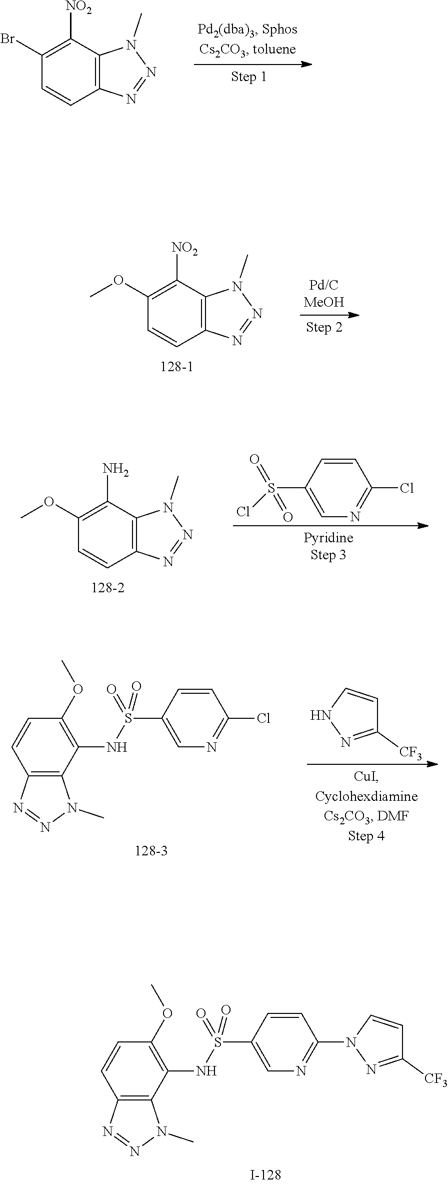

Step 1: Synthesis of 128-1. A mixture of 6-bromo-1-methyl-7-nitro-1,2,3-benzotriazole (420 mg, 1.63 mmol, 1 equiv.), Pd₂(dba)₃ (299.25 mg, 0.33 mmol, 0.2 equiv.), Sphos (134.16 mg, 0.327 mmol, 0.2 equiv.) and Cs₂CO₃ (1064.74 mg, 3.27 mmol, 2 equiv.) in toluene (20 mL) and MeOH (2 mL) was stirred overnight at 100° C. under a nitrogen atmosphere. After the reaction was completed, the solvent was removed under vacuum. The resulting mixture was diluted with water (100 mL) and then extracted with EtOAc (4×100 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water, 0% to 100% gradient in 25 min; detector: UV 254 nm to give 128-1 (100 mg, 29.40%) as a yellow solid.

Step 2: Synthesis of 128-2. A mixture of 128-1 (100 mg, 0.48 mmol, 1 equiv.) and Pd/C (50 mg, 0.470 mmol, 0.98 equiv.) in MeOH (5 mL) was stirred overnight at room temperature under a hydrogen atmosphere. After the reaction was completed, the solid was filtered. The filtrate was concentrated under vacuum to give 128-2 (130 mg) as a red solid.

Step 3: Synthesis of 128-3. A solution of 128-2 (82.2 mg, 0.46 mmol, 1 equiv.) and 6-chloropyridine-3-sulfonyl chloride (117.37 mg, 0.55 mmol, 1.2 equiv.) in pyridine (5 mL) was stirred overnight at room temperature. After the reaction was completed, the solvent was concentrated and the mixture was purified by purified by reversed-phase flash chromatography with the following conditions: column: C18 silica gel; mobile phase: ACN in water, 0% to 100% gradient in 20 min; detector: UV 254 nm to give 128-3 (86 mg, 52.70%) as a brown solid.

Step 4: Synthesis of N-(6-methoxy-1-methyl-1H-benzo[d][1,2,3]triazol-7-yl)-6-(3-(trifluoromethyl)-1H-pyrazol-1-yl)pyridine-3-sulfonamide (1-128). A solution of 128-3 (160.5 mg, 0.454 mmol, 1 equiv.), cyclohexane-1,4-diamine (51.80 mg, 0.454 mmol, 1 equiv.), 3-(trifluoromethyl)-1H-pyrazole (67.91 mg, 0.499 mmol, 1.1 equiv.), CuI (43.20 mg, 0.227 mmol, 0.5 equiv.) and Cs₂CO₃ (443.44 mg, 1.362 mmol, 3 equiv.) in DMF (14 mL) was stirred overnight at 140° C. under a nitrogen atmosphere. After the reaction was completed, the resulting mixture was diluted with water (100 mL) and then extracted with EtOAc (4×100 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water, 0% to 100% gradient in 20 min; detector: UV 254 nm to give a crude product, which was purified by prep-HPLC with the following conditions: column: XBridge Prep OBD Cis, 30*150 mm, 5 m; mobile phase A: 10 mmolNH₄HCO₃+0.05% NH₃H₂O, mobile phase B: ACN; flow rate 60 mL/min; gradient: 22% B to 47% B in 8 min, 47% B; wavelength: 220 nm; Rt1(min): 8.42) to afford N-(6-methoxy-1-methyl-1H-benzo[d][1,2,3]triazol-7-yl)-6-(3-(trifluoromethyl)-1H-pyrazol-1-yl)pyridine-3-sulfonamide (15.7 mg, 7.60%) as a pink solid. LCMS: (ES, m/z): [M+H]⁺ 454; ¹H NMR (400 MHz, DMSO-d₆) δ 3.25 (s, 3H), 4.48 (s, 3H), 7.10-7.16 (m, 2H), 7.98 (d, J9.2 Hz, 1H), 8.17 (d, J8.8 Hz, 1H), 8.28 (dd, J=8.8, 2.4 Hz, 1H), 8.69 (d, J2.0 Hz, 1H), 8.94 (s, 1H), 10.41 (s, 1H).

### Example 198—Synthesis of 6-(3,4-Dimethyl-1H-Pyrazol-1-yl)-N-(6-Methoxy-1-Methyl-1H-Indazol-7-yl)Pyridine-3-Sulfonamide (I-129)

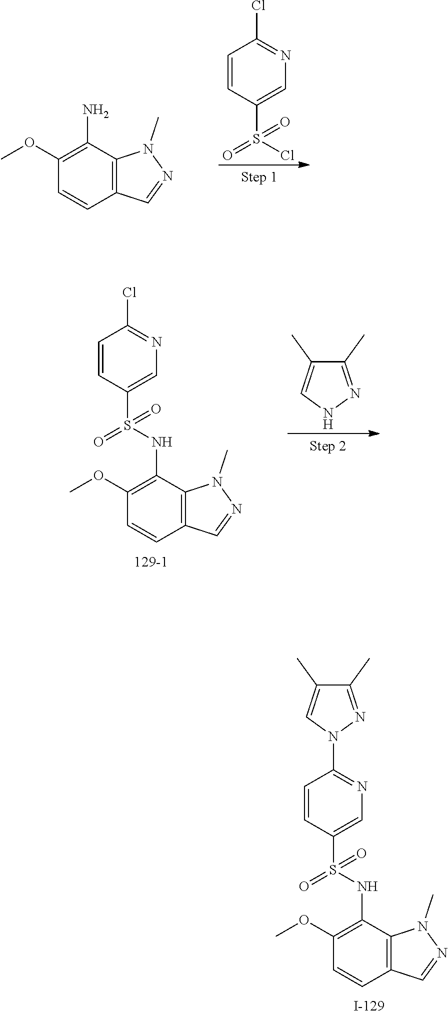

Step 1: Synthesis of 129-1. To a stirred solution of 6-methoxy-1-methylindazol-7-amine (2 g, 11.286 mmol, 1 equiv.) and 6-chloropyridine-3-sulfonyl chloride (2.63 g, 12.415 mmol, 1.1 equiv.) in MeCN (6 mL) was added pyridine (4.80 mL, 59.703 mmol, 5.29 equiv.) dropwise at room temperature under a nitrogen atmosphere. The resulting mixture was stirred for an additional 2 h at room temperature. The mixture was diluted with water (50 mL) and extracted with EtOAc (3×50 mL). The combined organic layers were washed with water (100 mL) and dried over anhydrous Na₂SO₄. After filtration, the filtrate was concentrated under reduced pressure. This resulted in 6-chloro-N-(6-methoxy-1-methylindazol-7-yl)pyridine-3-sulfonamide (3 g, crude) as a light yellow solid.

Step 2: Synthesis of 6-(3,4-dimethyl-1H-pyrazol-1-yl)-N-(6-methoxy-1-methyl-1H-indazol-7-yl)pyridine-3-sulfonamide (1-129). To a stirred solution of 6-chloro-N-(6-methoxy-1-methylindazol-7-yl)pyridine-3-sulfonamide (200 mg, 0.567 mmol, 1 equiv.) and t-BuOK (190.84 mg, 1.701 mmol, 3 equiv.) in DMSO (6 mL) were added 3,4-dimethyl-1H-pyrazole (109.00 mg, 1.134 mmol, 2 equiv.) dropwise at room temperature under a nitrogen atmosphere. The resulting mixture was stirred overnight at 140° C. The resulting mixture was diluted with water (10 mL). The resulting mixture was extracted with EtOAc (3×10 mL). The combined organic layers were washed with brine (2×30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 20% to 80% gradient in 20 min; detector: UV 254 nm. The crude product (170 mg) was purified by prep-HPLC with the following conditions: column: XBridge Prep OBD C18, 30*150 mm, 5 m; mobile phase A: water (10 mmol/L NH₄HCO₃+0.1% NH₃. H₂O), mobile phase B: ACN; flow rate 60 mL/min; gradient: 25% B to 51% B in 11 min, 51% B; wavelength: 220 nm; Rt1(min): 10.53; number of runs: 0) to afford 6-(3,4-dimethylpyrazol-1-yl)-N-(6-methoxy-1-methylindazol-7-yl)pyridine-3-sulfonamide (33.4 mg, 14.23%) as a off-white solid. LCMS: (ES, m/z): [M+H]⁺ 413; ¹H NMR (400 MHz, DMSO-d₆) δ 2.05 (s, 3H), 2.23 (s, 3H), 3.17 (s, 3H), 4.27 (s, 3H), 6.82 (d, J 8.8 Hz, 1H), 7.67 (d, J 8.8 Hz, 1H), 7.98 (d, J 8.8 Hz, 1H), 8.03 (s, 1H), 8.11 (dd, J 8.8, 2.4 Hz, 1H), 8.39 (s, 1H), 8.53 (d, J 2.3 Hz, 1H), 9.91 (s, 1H).

### Example 199—Synthesis of 5′-Cyano-N-(1-Methyl-1H-Indazol-7-yl)-[2,3′-Bipyridine]-5-Sulfonamide (I-130)

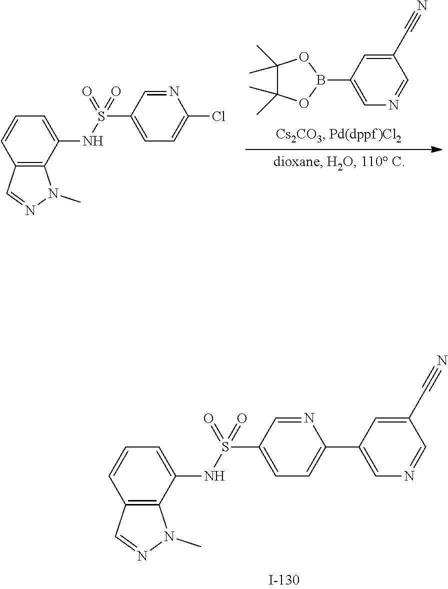

To a stirred mixture of 6-chloro-N-(1-methylindazol-7-yl)pyridine-3-sulfonamide (100 mg, 0.310 mmol, 1 equiv.) and 5-(4,4,5,5-tetramethyl-1,3,2-dioxaborolan-2-yl)pyridine-3-carbonitrile (106.92 mg, 0.465 mmol, 1.5 equiv.) in H₂O (1 mL) and 1,4-dioxane (4 mL) were added XPhos Pd G3 (52.45 mg, 0.062 mmol, 0.2 equiv.) and Cs₂CO₃ (302.83 mg, 0.930 mmol, 3 equiv.) at room temperature under a nitrogen atmosphere. The resulting mixture was stirred overnight at 110° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. The resulting mixture was concentrated under vacuum. This resulted in 5′-cyano-N-(1-methylindazol-7-yl)-[2,3′-bipyridine]-5-sulfonamide (15.3 mg, 12.19%) as an off-white solid. LCMS: (ES, m/z): [M+H]⁺ 391; ¹H NMR (400 MHz, DMSO-d₆) δ4.30 (s, 3H), 6.52 (d, J7.2 Hz, 1H), 6.92 (t, J7.6 Hz, 1H), 7.68 (d, J8.0 Hz, 1H), 8.09 (s, 1H), 8.22 (dd, J2.4, 8.4 Hz, 1H), 8.42 (d, J8.4 Hz, 1H), 8.93 (d, J2.4 Hz, 1H), 9.04 (t, J2.0 Hz, 1H), 9.17 (d, J2.0 Hz, 1H), 9.60 (d, J2.4 Hz, 1H), 10.42 (s, 1H).

### Example 200—Synthesis of 5′-Methyl-N-(1-Methyl-1H-Indazol-7-yl)-[2,3′-Bipyridine]-5-Sulfonamide (I-131)

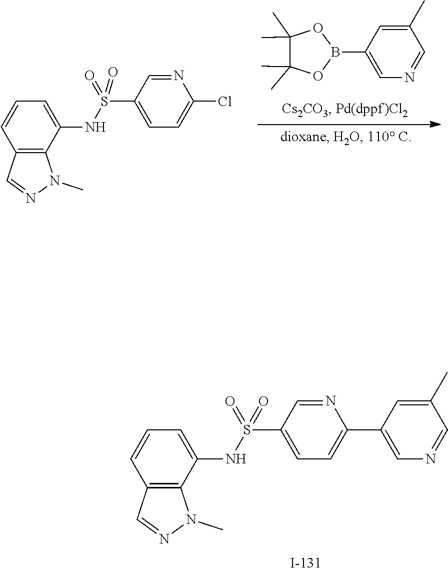

To a stirred mixture of 6-chloro-N-(1-methylindazol-7-yl)pyridine-3-sulfonamide (100 mg, 0.310 mmol, 1 equiv.) and 3-methyl-5-(4,4,5,5-tetramethyl-1,3,2-dioxaborolan-2-yl)pyridine (101.82 mg, 0.465 mmol, 1.5 equiv.) in H₂O (1 mL) and 1,4-dioxane (4 mL) were added Pd(dppf)Cl2 (45.34 mg, 0.062 mmol, 0.2 equiv.) and Cs₂CO₃ (302.83 mg, 0.930 mmol, 3 equiv.) at room temperature under a nitrogen atmosphere. The resulting mixture was stirred overnight at 110° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. The resulting mixture was concentrated under vacuum. This resulted in 5′-methyl-N-(1-methylindazol-7-yl)-[2,3′-bipyridine]-5-sulfonamide (33.1 mg, 28.10%) as an off-white solid. LCMS:(ES, m/z): [M+H]⁺ 380; ¹H NMR (400 MHz, DMSO-d₆) δ 2.41 (s, 3H), 4.29 (s, 3H), 6.52 (d, J7.2 Hz, 1H), 6.93 (t, J 7.6 Hz, 1H), 7.69 (d, J8.2 Hz, 1H), 8.10 (s, 1H), 8.14 (dd, J2.4, 8.4 Hz, 1H), 8.31 (d, J8.4 Hz, 1H), 8.37 (s, 1H), 8.57 (d, J2.0 Hz, 1H), 8.87 (d, J2.4 Hz, 1H), 9.15 (d, J2.0 Hz, 1H), 10.36 (s, 1H).

### Example 201—Synthesis of 2′-Methyl-N-(1-Methyl-1H-Indazol-7-yl)-[2,4′-Bipyridine]-5-Sulfonamide (I-132)

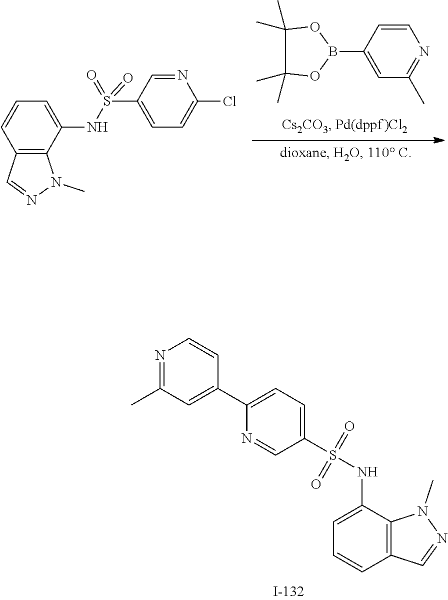

To a stirred solution of 6-chloro-N-(1-methylindazol-7-yl)pyridine-3-sulfonamide (100 mg, 0.310 mmol, 1 equiv.) and 2-methyl-4-(4,4,5,5-tetramethyl-1,3,2-dioxaborolan-2-yl)pyridine (67.88 mg, 0.310 mmol, 1 equiv.) in H₂O (1 mL) and 1,4-dioxane (4 mL) were added Pd(dppf)Cl₂ (45.34 mg, 0.062 mmol, 0.2 equiv.) and Cs₂CO₃ (302.83 mg, 0.930 mmol, 3 equiv.) under a nitrogen atmosphere. The resulting mixture was stirred overnight at 110° C. under a nitrogen atmosphere. The resulting mixture was diluted with water (10 mL). The resulting mixture was extracted with CH₂Cl₂ (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 2′-methyl-N-(1-methylindazol-7-yl)-[2,4′-bipyridine]-5-sulfonamide (32.2 mg, 27.20%) as a white solid. LCMS: (ES, m/z): [M+H]⁺ 380; (400 MHz, DMSO-d₆) δ 2.58 (s, 3H), 4.29 (s, 3H), 6.52 (d, J 7.2 Hz, 1H), 6.91 (t, J 7.8 Hz, 1H), 7.63 (s, 1H), 7.91 (dd, J 5.2, 1.6 Hz, 1H), 8.01 (s, 1H), 8.08 (s, 1H), 8.18 (dd, J 8.4, 2.4 Hz, 1H), 8.33 (d, J 8.4 Hz, 1H), 8.63 (d, J 5.2 Hz, 1H), 8.91 (dd, J 2.4, 0.8 Hz, 1H), 10.39 (s, 1H).

### Example 202—Synthesis of N-(1-Methyl-1H-Indazol-7-yl)-6-(Pyrimidin-5-yl)Pyridine-3-Sulfonamide (I-133)

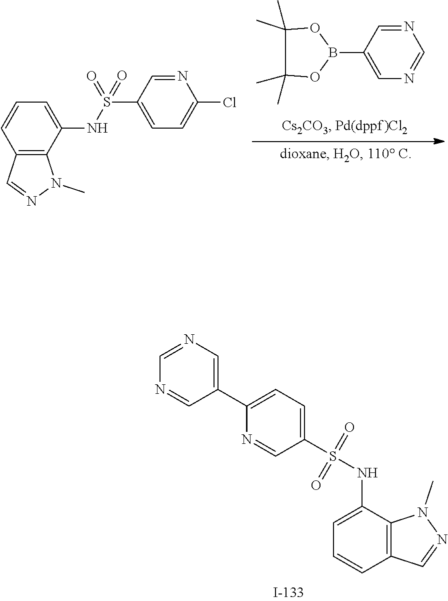

To a stirred mixture of 6-chloro-N-(1-methylindazol-7-yl)pyridine-3-sulfonamide (100 mg, 0.310 mmol, 1 equiv.) and 5-(4,4,5,5-tetramethyl-1,3,2-dioxaborolan-2-yl)pyrimidine (63.84 mg, 0.310 mmol, 1 equiv.) in dioxane (4 mL) and H₂O (1 mL) were added Pd(dppf)Cl₂ (45.34 mg, 0.062 mmol, 0.2 equiv.) and Cs₂CO₃ (302.83 mg, 0.930 mmol, 3 equiv.) at room temperature under a nitrogen atmosphere. The resulting mixture was stirred overnight at 110° C. under a nitrogen atmosphere. The resulting mixture was diluted with water (10 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 30 min; detector: UV 254 nm. The resulting mixture was concentrated under vacuum. This resulted in N-(1-methylindazol-7-yl)-6-(pyrimidin-5-yl)pyridine-3-sulfonamide (12.8 mg, 11.24%) as a white solid. LCMS: (ES, m/z): [M+H]⁺ 367; (400 MHz, DMSO-d₆) δ 4.30 (s, 3H), 6.53 (d, J 7.2 Hz, 1H), 6.92 (t, J 7.6 Hz, 1H), 7.67 (d, J 8.0 Hz, 1H), 8.09 (s, 1H), 8.21 (dd, J 8.4, 2.4 Hz, 1H), 8.40 (d, J 8.4 Hz, 1H), 8.93 (d, J 2.4 Hz, 1H), 9.33 (s, 1H), 9.53 (s, 2H), 10.42 (s, 1H).

### Example 203—Synthesis of 5′-Fluoro-N-(1-Methyl-1H-Indazol-7-yl)-[2,3′-Bipyridine]-5-Sulfonamide (I-134)

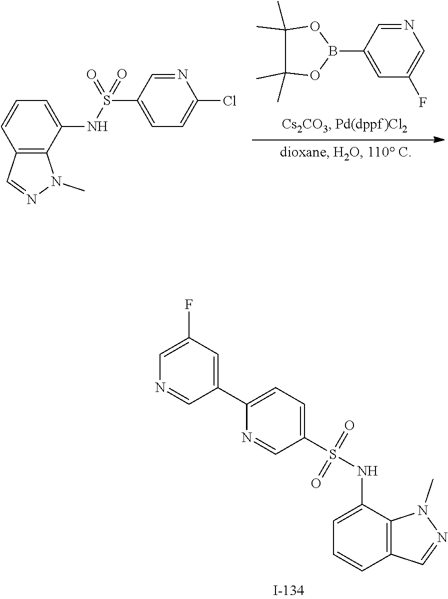

To a stirred solution of 6-chloro-N-(1-methylindazol-7-yl)pyridine-3-sulfonamide (100 mg, 0.310 mmol, 1 equiv.) and 3-fluoro-5-(4,4,5,5-tetramethyl-1,3,2-dioxaborolan-2-yl)pyridine (63.53 mg, 0.310 mmol, 1 equiv.) in H₂O (1 mL) and 1,4-dioxane (4 mL) were added Pd(dppf)Cl₂ (45.34 mg, 0.062 mmol, 0.2 equiv.) and Cs₂CO₃ (302.83 mg, 0.930 mmol, 3 equiv.) under a nitrogen atmosphere. The resulting mixture was stirred overnight at 110° C. under a nitrogen atmosphere. The resulting mixture was diluted with water (10 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 5′-fluoro-N-(1-methylindazol-7-yl)-[2,3′-bipyridine]-5-sulfonamide (19.9 mg, 16.70%) as a white solid. LCMS: (ES, m/z): [M+H]⁺ 384; (400 MHz, DMSO-d₆) δ 4.29 (s, 3H), 6.52 (dd, J 7.2, 1.2 Hz, 1H), 6.93 (t, J 7.6 Hz, 1H), 7.70 (d, J 8.0 Hz, 1H), 8.11 (s, 1H), 8.18 (dd, J 8.4, 2.4 Hz, 1H), 8.39 (d, J 8.4 Hz, 1H), 8.46 (dd, J 8.4, 2.0 Hz, 1H), 8.75 (d, J 2.8 Hz, 1H), 8.91 (dd, J 2.4, 0.8 Hz, 1H), 9.26 (t, J 1.6 Hz, 1H), 10.40 (s, 1H).

### Example 204—Synthesis of N-(1-Methyl-1H-Indazol-7-yl)-6-(2-Methylpyrimidin-5-yl)Pyridine-3-Sulfonamide (I-135)

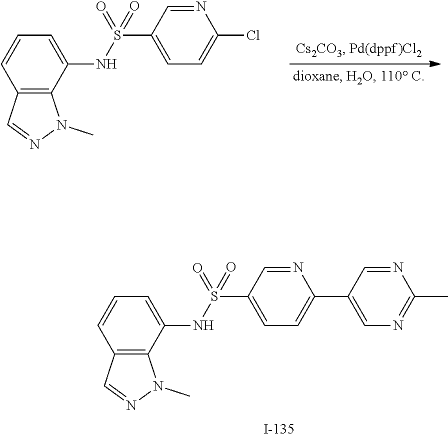 [SMILES: Cn1ncc2cccc(NS(=O)(=O)c3ccc(Cl)nc3)c21 ; Cc1ncc(-c2ccc(S(=O)(=O)Nc3cccc4cnn(C)c34)cn2)cn1]

To a stirred mixture of 6-chloro-N-(1-methylindazol-7-yl)pyridine-3-sulfonamide (100 mg, 0.310 mmol, 1 equiv.) and 2-methyl-5-(4,4,5,5-tetramethyl-1,3,2-dioxaborolan-2-yl)pyrimidine (102.28 mg, 0.465 mmol, 1.5 equiv.) in H₂O (1 mL) and 1,4-dioxane (4 mL) were added Pd(dppf)Cl₂ (45.34 mg, 0.062 mmol, 0.2 equiv.) and Cs₂CO₃ (302.83 mg, 0.930 mmol, 3 equiv.) at room temperature under a nitrogen atmosphere. The resulting mixture was stirred overnight at 110° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. The resulting mixture was concentrated under vacuum. This resulted in N-(1-methylindazol-7-yl)-6-(2-methylpyrimidin-5-yl)pyridine-3-sulfonamide (20.1 mg, 17.00%) as an off-white solid. LCMS:(ES, m/z): [M+H]⁺381; ¹H NMR (400 MHz, DMSO-d₆) δ 2.73 (d, J2.8 Hz, 3H), 4.29 (d, J2.8 Hz, 3H), 6.52 (d, J 7.2 Hz, 1H), 6.94 (t, J 7.2 Hz, 1H), 7.71 (d, J 8.0 Hz, 1H), 8.10 (s, 1H), 8.18 (d, J 8.4 Hz, 1H), 8.37 (d, J8.4 Hz, 1H), 8.91 (s, 1H), 9.41 (s, 1H), 9.51 (s, 1H), 10.40 (s, 1H).

### Example 205—Synthesis of 6′-Hydroxy-N-(1-Methyl-1H-Indazol-7-yl)-[2,3′-Bipyridine]-5-Sulfonamide (I-136)

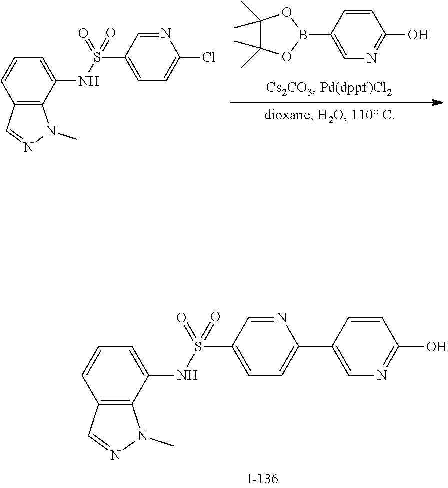

To a stirred mixture of 6-chloro-N-(1-methylindazol-7-yl)pyridine-3-sulfonamide (100 mg, 0.310 mmol, 1 equiv.) and 5-(4,4,5,5-tetramethyl-1,3,2-dioxaborolan-2-yl)pyridin-2-ol (102.73 mg, 0.465 mmol, 1.5 equiv.) in H₂O (1 mL) and 1,4-dioxane (4 mL) were added Pd(dppf)Cl₂ (45.34 mg, 0.062 mmol, 0.2 equiv.) and Cs₂CO₃ (302.83 mg, 0.930 mmol, 3 equiv.) at room temperature under a nitrogen atmosphere. The resulting mixture was stirred overnight at 110° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. The resulting mixture was concentrated under vacuum. This resulted in 6′-hydroxy-N-(1-methylindazol-7-yl)-[2,3′-bipyridine]-5-sulfonamide (2.6 mg, 2.18%) as an off-white solid. LCMS:(ES, m/z): [M+H]⁺ 382; ¹H NMR (400 MHz, DMSO-d₆) δ 4.29 (s, 3H), 6.46 (d, J8.4 Hz, 1H), 6.52 (d, J7.2 Hz, 1H), 6.94 (t, J7.2 Hz, 1H), 7.50-7.58 (m, 1H), 7.84-8.12 (m, 3H), 8.23-8.28 (m, 2H), 8.72 (s, 1H), 10.24 (s, 1H), 12.11 (s, 1H).

### Example 206—Synthesis of N-(1-Methyl-1H-Indazol-7-yl)-5′-(Methylsulfonyl)-[2,3′-Bipyridine]-5-Sulfonamide (1-137)

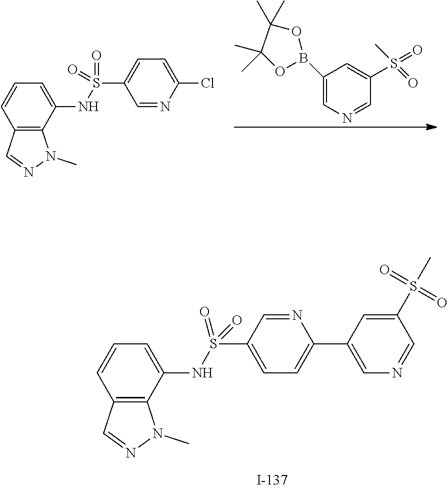

A solution of 6-chloro-N-(1-methylindazol-7-yl)pyridine-3-sulfonamide (100 mg, 0.310 mmol, 1 equiv.) in dioxane (3 mL) was treated with 3-methanesulfonyl-5-(4,4,5,5-tetramethyl-1,3,2-dioxaborolan-2-yl)pyridine (131.59 mg, 0.465 mmol, 1.5 equiv.) for 3 min at room temperature under a nitrogen atmosphere followed by the addition of K₃PO₄ (197.29 mg, 0.930 mmol, 3 equiv.), XPhos Pd G3 (118.16 mg, 0.248 mmol, 0.8 equiv.) and XPhos (59.08 mg, 0.124 mmol, 0.4 equiv.) in portions. The resulting mixture was stirred for an additional 3 h at 60° C. The resulting mixture was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. This resulted in N-(1-methyl-1H-indazol-7-yl)-5′-(methylsulfonyl)-[2,3′-bipyridine]-5-sulfonamide (6.5 mg, 4.72%) as a white solid. LCMS: (ES, m/z): [M+H]⁺444; (400 MHz, DMSO-d₆): δ 3.43 (s, 3H), 4.32 (s, 3H), 6.59 (d, J7.2 Hz, 1H), 6.88 (t, J7.2 Hz, 1H), 7.54 (s, 1H), 8.04 (s, 1H), 8.23 (d, J8.0 Hz, 1H), 8.44 (d, J8.4 Hz, 1H), 8.94-9.00 (m, 2H), 9.19 (s, 1H), 9.64 (s, 1H), 10.39 (s, 1H).

### Example 207—Synthesis of 6-(1-(Difluoromethyl)-1H-Pyrazol-3-yl)-N-(1-Methyl-1H-Indazol-7-yl)Pyridine-3-Sulfonamide (I-138)

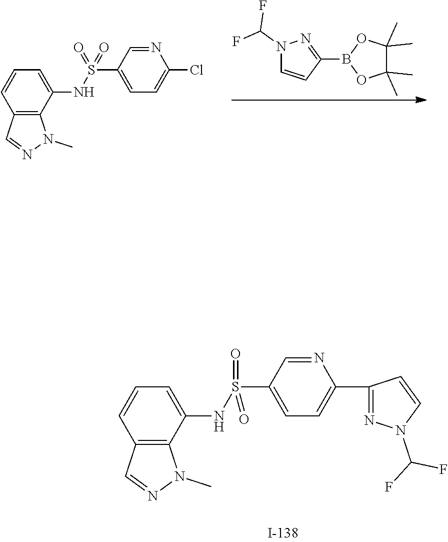 [SMILES: Cn1ncc2cccc(NS(=O)(=O)c3ccc(Cl)nc3)c21 ; CC1(C)OB(c2ccn(C(F)F)n2)OC1(C)C ; Cn1ncc2cccc(NS(=O)(=O)c3ccc(-c4ccn(C(F)F)n4)nc3)c21]

A mixture of 6-chloro-N-(1-methylindazol-7-yl)pyridine-3-sulfonamide (100.00 mg, 0.310 mmol, 1 equiv.), 1-(difluoromethyl)-3-(4,4,5,5-tetramethyl-1,3,2-dioxaborolan-2-yl)-1H-pyrazole (302.44 mg, 1.240 mmol, 4 equiv.), Pd(dppf)Cl₂CH₂Cl₂ (50.48 mg, 0.062 mmol, 0.2 equiv.) and Cs₂CO₃ (302.83 mg, 0.930 mmol, 3 equiv.) in H₂O (1 mL) and dioxane (4 mL) was stirred for 16 h at 100° C. under a nitrogen atmosphere. The resulting mixture was diluted with water (10 mL). The resulting mixture was extracted with EtOAc (3×15 mL). The combined organic layers were washed with brine (10 mL) and dried over anhydrous Na₂SO₄. After filtration, the filtrate was concentrated under reduced pressure. The crude product was purified by prep-HPLC with the following conditions: Column: XBridge Prep OBD C18, 30*150 mm, 5 m; mobile phase A: 10 mmol NH₄HCO₃+0.05% NH₃H₂O, mobile phase B: ACN; flow rate 60 mL/min; gradient: 10% B to 35% B in 8 min, 35% B; wavelength: 220 nm; Rt1(min): 8.13, to afford 6-(1-(difluoromethyl)-1H-pyrazol-3-yl)-N-(1-methyl-1H-indazol-7-yl)pyridine-3-sulfonamide (49.60 mg, 39.11%) as an off-white solid. LCMS: (ES, m/z): [M+H]⁺ 405; (400 MHz, DMSO-d₆) δ 4.28 (s, 3H), 6.51 (d, J 7.2 Hz, 1H), 6.92 (t, J 7.6 Hz, 1H), 7.18 (d, J 2.8 Hz, 1H), 7.67 (d, J 8.0 Hz, 1H), 7.76 (d, J 8.0 Hz, 1H), 8.09 (s, 1H), 8.14 (d, J 8.4 Hz, 1H), 8.22 (d, J 8.4 Hz, 1H), 8.42 (d, J 2.8 Hz, 1H), 8.81 (d, J 2.4 Hz, 1H), 10.35 (s, 1H).

### Example 208—Synthesis of 2′-Methoxy-N-(1-Methyl-1H-Indazol-7-yl)-[2,3′-Bipyridine]-5-Sulfonamide (1-139)

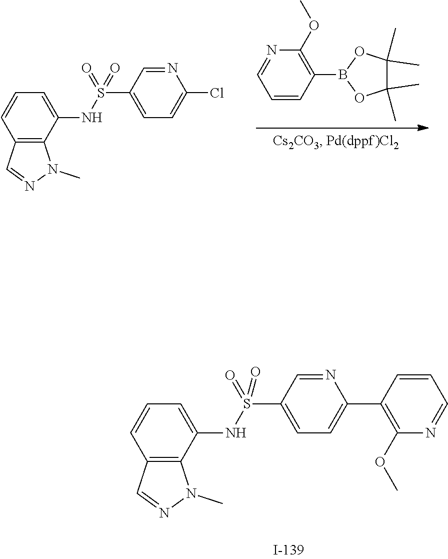 [SMILES: Cn1ncc2cccc(NS(=O)(=O)c3ccc(Cl)nc3)c21 ; COc1ncccc1B1OC(C)(C)C(C)(C)O1 ; COc1ncccc1-c1ccc(S(=O)(=O)Nc2cccc3cnn(C)c23)cn1]

A solution of 6-chloro-N-(1-methylindazol-7-yl)pyridine-3-sulfonamide (100 mg, 0.310 mmol, 1 equiv.) 2-methoxy-3-(4,4,5,5-tetramethyl-1,3,2-dioxaborolan-2-yl)pyridine (109.25 mg, 0.465 mmol, 1.5 equiv.), Pd(dppf)Cl₂·CH₂Cl₂ (25.24 mg, 0.031 mmol, 0.1 equiv.) and Cs₂CO₃ (302.83 mg, 0.930 mmol, 3 equiv.) in dioxane (15 mL) and H₂O (5 mL) was stirred overnight at 90° C. under a nitrogen atmosphere. The resulting mixture was extracted with EtOAc (2×20 mL). The resulting mixture was diluted with water (10 mL). The resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. The resulting mixture was concentrated under reduced pressure. This resulted in 2′-methoxy-N-(1-methylindazol-7-yl)-[2,3′-bipyridine]-5-sulfonamide (12.0 mg, 9.78%) as an off-white solid. LCMS: (ES, m/z): [M+H]⁺ 396; (400 MHz, DMSO-d₆) δ 4.00 (s, 3H), 4.29 (s, 3H), 6.54 (d, J7.2 Hz, 1H), 6.94 (t, J7.6 Hz, 1H), 7.21 (d, J7.6, 4.8 Hz, 1H), 7.68 (d, J 8.0 Hz, 1H), 8.07-8.17 (m, 2H), 8.27 (d, J8.8 Hz, 1H), 8.30-8.40 (m, 2H), 8.86 (d, J2.0 Hz, 1H), 10.35 (s, 1H).

### Example 209—Synthesis of N-(6-Methoxy-1-Methyl-1H-Indazol-7-yl)-6-(3,4,5-Trimethyl-1H-Pyrazol-1-yl)Pyridine-3-Sulfonamide (I-140)

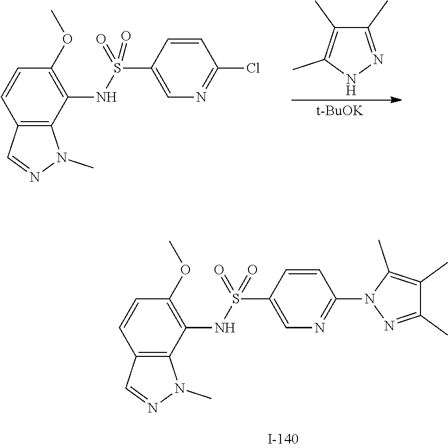

A mixture of 6-chloro-N-(6-methoxy-1-methyl-1H-indazol-7-yl)pyridine-3-sulfonamide (100.00 mg, 0.283 mmol, 1 equiv.), 3,4,5-trimethyl-1H-pyrazole (40.59 mg, 0.368 mmol, 1.3 equiv.) and t-BuOK (95.42 mg, 0.849 mmol, 3 equiv.) in DMSO (4 mL) was stirred for 2 h at 140° C. under a nitrogen atmosphere. The resulting mixture was diluted with water (10 mL). The resulting mixture was extracted with EtOAc (3×15 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: 10 mmol NH₄HCO₃+0.05% NH₃H₂O, mobile phase B: ACN; flow rate 60 mL/min; gradient: 34% B to 59% B in 8 min, 59% B; wavelength: 220 nm; Rt1(min): 7.63; injection volume: 0.7 mL, to afford N-(6-methoxy-1-methyl-1H-indazol-7-yl)-6-(3,4,5-trimethyl-1H-pyrazol-1-yl)pyridine-3-sulfonamide (1.80 mg, 1.49%) as an off-white solid. LCMS: (ES, m/z): [M+H]⁺ 427; (400 MHz, DMSO-d₆) δ 1.95 (s, 3H), 2.19 (s, 3H), 2.56 (s, 3H), 3.19 (s, 3H), 4.27 (s, 3H), 6.83 (d, J 8.8 Hz, 1H), 7.66 (d, J 8.8 Hz, 1H), 7.96 (d, J 2.4 Hz, 1H), 7.98 (s, 1H), 8.07 (d, J 2.4 Hz, 1H), 8.55 (d, J 2.4 Hz, 1H), 9.87 (s, 1H).

### Example 210—Synthesis of 6-(4-Chloro-3,5-Dimethyl-1H-Pyrazol-1-yl)-N-(6-Methoxy-1-Methyl-1H-Indazol-7-yl)Pyridine-3-Sulfonamide (I-141)

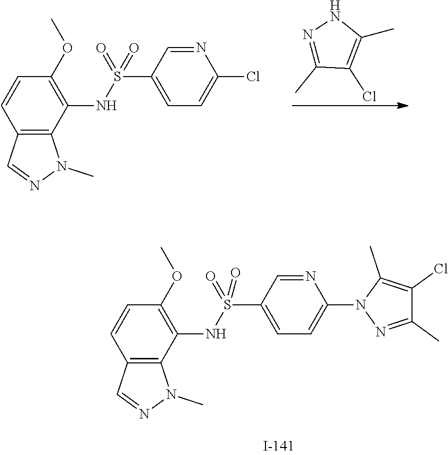

A solution of 6-chloro-N-(6-methoxy-1-methylindazol-7-yl)pyridine-3-sulfonamide (100 mg, 0.283 mmol, 1 equiv.) and 4-chloro-3,5-dimethyl-1H-pyrazole (123 mg, 0.845 mmol, 2.98 equiv.) and t-BuOK (95 mg, 0.847 mmol, 2.99 equiv.) in DMSO (5 mL) was stirred overnight at 140° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃), mobile phase B: ACN; flow rate 60 mL/min; gradient: 46% B to 66% B in 8 min, 66% B; wavelength: 254 nm; Rt1(min): 7.17; injection volume: 0.9 mL; number of runs: 2) to afford 6-(4-chloro-3,5-dimethylpyrazol-1-yl)-N-(6-methoxy-1-methylindazol-7-yl) pyridine-3-sulfonamide (4.8 mg, 3.77%) as an off-white solid. LCMS: (ES, m/z): [M+H]⁺ 448; ¹H NMR (400 MHz, DMSO-d₆) δ 2.27 (s, 3H), 2.64 (s, 3H), 3.18 (s, 3H), 4.27 (s, 3H), 6.82 (d, J8.8 Hz, 1H), 7.65 (d, J9.2 Hz, 1H), 7.99 (s, 1H), 8.01 (d, J=8.4 Hz, 1H), 8.17 (dd, J8.8, 2.4 Hz, 1H), 8.61 (d, J2.4 Hz, 1H), 10.05 (s, 1H).

### Example 211—Synthesis of 6-(3,5-Dimethyl-1H-Pyrazol-1-yl)-N-(6-Methoxy-1-Methyl-1H-Indazol-7-yl)Pyridine-3-Sulfonamide (I-142)

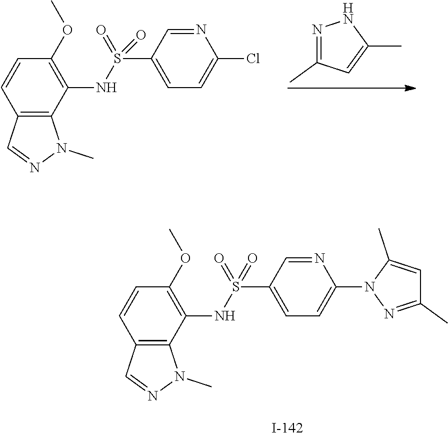

A solution of 6-chloro-N-(6-methoxy-1-methylindazol-7-yl)pyridine-3-sulfonamide (100 mg, 0.283 mmol, 1 equiv.) and 3,5-dimethylpyrazol-1-amine (95 mg, 0.855 mmol, 3.02 equiv.) and t-BuOK (95 mg, 0.847 mmol, 2.99 equiv.) in DMSO (5 mL) was stirred overnight at 140° C. under a nitrogen atmosphere. The resulting mixture was diluted with water (10 mL) and then extracted with EtOAc (3×50 mL). The combined organic layers were washed with brine (3×10 mL) and dried over anhydrous Na₂SO₄. After filtration, the filtrate was concentrated under reduced pressure. The crude product (50 mg) was purified by prep-HPLC with the following conditions: column: XBridge Prep OBD C18, 30*150 mm, 5 m; mobile phase A: water (10 mmol NH₄HCO₃+0.05% NH₃H₂O), mobile phase B: ACN; flow rate 60 mL/min; gradient: 29% B to 54% B in 8 min, 54% B; wavelength: 220 nm; Rt1(min): 7.80; injection volume: 0.5 mL; number of runs: 42) to afford 6-(3,5-dimethyl-1H-pyrazol-1-yl)-N-(6-methoxy-1-methyl-1H-indazol-7-yl)pyridine-3-sulfonamide (56.6 mg, 48.41%) as an off-white solid. LCMS: (ES, m/z): [M+H]⁺ 413; ¹H NMR (400 MHz, DMSO-d₆) δ2.23 (s, 3H), 2.63 (s, 3H), 3.20 (s, 3H), 4.27 (s, 3H), 6.21 (s, 1H), 6.84 (d, J8.8 Hz, 1H), 7.68 (d, J8.8 Hz, 1H), 7.99-8.02 (m, 2H), 8.12 (dd, J8.8, 2.4 Hz, 1H), 8.57 (d, J2.4 Hz, 1H), 9.99 (s, 1H).

### Example 212—Synthesis of N-(6-Methoxy-1-Methyl-1H-Indazol-7-yl)-N-Methyl-6-(3-(Trifluoromethyl)-1H-Pyrazol-5-yl)Pyridine-3-Sulfonamide (I-48)

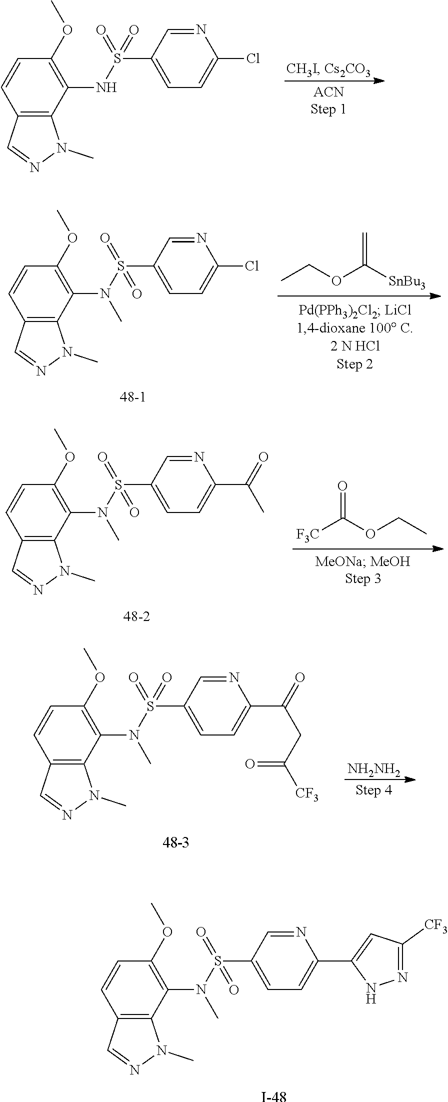

Step 1: Synthesis of 48-1. A solution of 6-chloro-N-(6-methoxy-1-methylindazol-7-yl)pyridine-3-sulfonamide (500 mg, 1.417 mmol, 1 equiv.), Cs₂CO₃ (923.55 mg, 2.834 mmol, 2 equiv.) and CH₃I (301.75 mg, 2.126 mmol, 1.5 equiv.) in ACN (14 mL) was stirred for 1 h at room temperature. The reaction was quenched by the addition of water (50 mL) at room temperature. The aqueous layer was extracted with EtOAc (3×50 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give 6-chloro-N-(6-methoxy-1-methylindazol-7-yl)-N-methylpyridine-3-sulfonamide (500 mg, 94.25%) as yellow solid.

Step 2: Synthesis of 48-2. A solution of 6-chloro-N-(6-methoxy-1-methylindazol-7-yl)-N-methylpyridine-3-sulfonamide (490 mg, 1.336 mmol, 1 equiv.), Pd(dppf)Cl2 (195.48 mg, 0.267 mmol, 0.2 equiv.), LiCl (113.25 mg, 2.672 mmol, 2 equiv.) and tributyl(1-ethoxyethenyl)stannane (964.87 mg, 2.672 mmol, 2 equiv.) in 1,4-dioxane (10 mL) was stirred for 3 h at 100° C. under a nitrogen atmosphere. The mixture was stirred with 2 M HCl for 30 min. The reaction was quenched with KF (aq.) at room temperature. The resulting mixture was extracted with EtOAc (3×50 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give 6-acetyl-N-(6-methoxy-1-methylindazol-7-yl)-N-methylpyridine-3-sulfonamide (338 mg, 67.58%) as brown solid.

Step 3: Synthesis of 48-3. A solution of 6-acetyl-N-(6-methoxy-1-methylindazol-7-yl)-N-methylpyridine-3-sulfonamide (330 mg, 0.881 mmol, 1 equiv.), trifluoroethyl acetate (250.44 mg, 1.762 mmol, 2 equiv.) and MeONa (476.15 mg, 8.810 mmol, 10 equiv.) in MeOH (8 mL) was stirred for 3 h at 70° C. The resulting mixture was diluted with water (50 mL). The resulting mixture was extracted with EtOAc (3×50 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give N-(6-methoxy-1-methylindazol-7-yl)-N-methyl-6-(4,4,4-trifluoro-3-oxobutanoyl)pyridine-3-sulfonamide (223 mg, 53.79%) as a yellow solid.

Step 4: Synthesis of N-(6-methoxy-1-methyl-1H-indazol-7-yl)-N-methyl-6-(3-(trifluoromethyl)-1H-pyrazol-5-yl)pyridine-3-sulfonamide (I-48). A solution of N-(6-methoxy-1-methylindazol-7-yl)-N-methyl-6-(4,4,4-trifluoro-3-oxobutanoyl)pyridine-3-sulfonamide (110 mg, 0.234 mmol, 1 equiv.) and NH₂NH₂. H₂O (58.53 mg, 1.170 mmol, 5 equiv.) in EtOH (3 mL) was stirred overnight at room temperature. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by prep-HPLC with the following conditions: column: XBridge Prep OBD C18, 30*150 mm, 5 m; mobile phase A: water (10 mmol/L NH₄HCO₃), mobile phase B: ACN; flow rate 60 mL/min; gradient: 37% B to 59% B in 8 min; wavelength: 254 nm; Rt1(min): 7.38; injection volume: 1 mL; number of runs: 3) to afford N-(6-methoxy-1-methylindazol-7-yl)-N-methyl-6-[5-(trifluoromethyl)-2H-pyrazol-3-yl]pyridine-3-sulfonamide (24 mg, 21.43%) as a white solid. LCMS: (ES, m/z): [M+H]⁺ 467; ¹H NMR (400 MHz, DMSO-d₆) δ 3.20 (s, 3H), 3.38 (s, 3H), 4.23(d, J6.8 Hz, 3H), 6.89(d, J8.8 Hz, 1H), 7.58 (d, J8.8 Hz, 1H), 7.76 (d, J8.8 Hz, 1H), 8.21 (s, 1H), 8.27 (dd, J8.4 Hz, 2.0 Hz, 2H), 8.81 (d, J2.0 Hz, 1H), 14.62 (s, 1H),

### Example 213—Synthesis of N-(6-Methoxy-1-Methyl-1H-Indazol-7-yl)-6-(1-Methyl-5-(Trifluoromethyl)-1H-Pyrazol-3-yl)Pyridine-3-Sulfonamide (I-49)

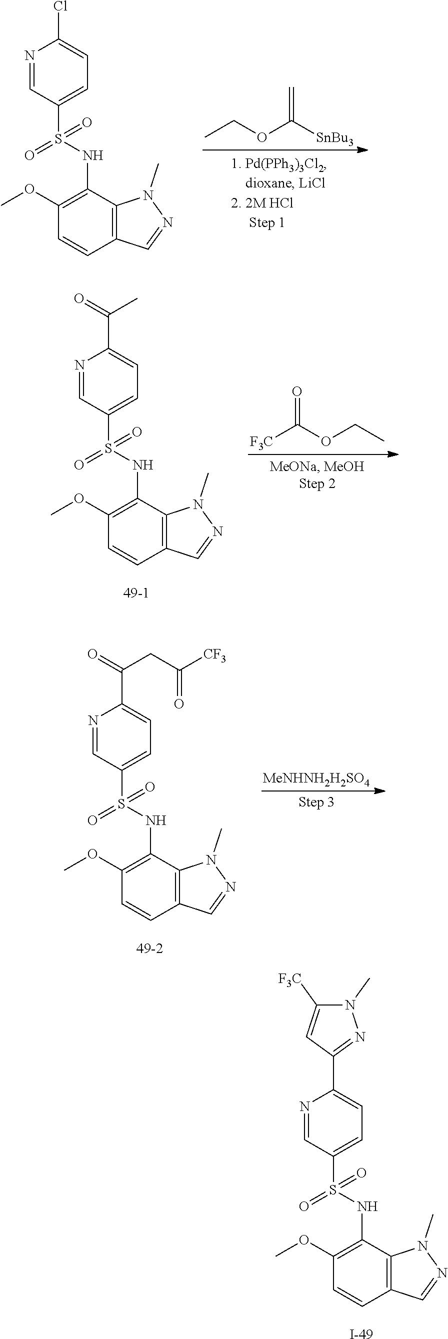

Step 1: Synthesis of 49-1. To a stirred solution of 6-chloro-N-(6-methoxy-1-methylindazol-7-yl)pyridine-3-sulfonamide (1.2 g, 3.401 mmol, 1 equiv.) and LiCl (288.38 mg, 6.802 mmol, 2 equiv.) in dioxane (20 mL) were added Pd(PPh₃)₂Cl₂ (477.50 mg, 0.680 mmol, 0.2 equiv.) and tributyl(1-ethoxyethenyl)stannane (2.30 mL, 6.802 mmol, 2 equiv.) at room temperature under a nitrogen atmosphere. The resulting mixture was stirred overnight at 100° C. under a nitrogen atmosphere. To the above mixture was added HCl (10 mL, 2 M). The resulting mixture was stirred for an additional 30 min at room temperature. The reaction was quenched with sat. KF (aq.) (20 mL) at room temperature. The resulting mixture was extracted with EtOAc (3×50 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 30 min; detector: UV 254 nm. This resulted in 6-acetyl-N-(6-methoxy-1-methylindazol-7-yl)pyridine-3-sulfonamide (750 mg, 55.06%) as a brown yellow solid.

Step 2: Synthesis of 49-2. A solution of 6-acetyl-N-(6-methoxy-1-methylindazol-7-yl)pyridine-3-sulfonamide (400 mg, 1.110 mmol, 1 equiv.) and trifluoroethyl acetate (0.26 mL, 2.220 mmol, 2 equiv.) in MeONa/MeOH (6 mL) was stirred for 3 h at 80° C. under a nitrogen atmosphere. The resulting mixture was diluted with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. This resulted in N-(6-methoxy-1-methylindazol-7-yl)-6-(4,4,4-trifluoro-3-oxobutanoyl)pyridine-3-sulfonamide (400 mg, 63.17%) as a yellow solid.

Step 3: Synthesis of N-(6-methoxy-1-methyl-1H-indazol-7-yl)-6-(1-methyl-5-(trifluoromethyl)-1H-pyrazol-3-yl)pyridine-3-sulfonamide (I-49). To a stirred solution of N-(6-methoxy-1-methylindazol-7-yl)-6-(4,4,4-trifluoro-3-oxobutanoyl) pyridine-3-sulfonamide (300 mg, 0.657 mmol, 1 equiv.) and methyl hydrazine sulfuric acid (189.50 mg, 1.314 mmol, 2 equiv.) in EtOH (4 mL) was added TEA (0.46 mL, 3.285 mmol, 5 equiv.). The resulting mixture was stirred for 24 h at 100° C. under a nitrogen atmosphere. The resulting mixture was diluted with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The crude product was purified by prep-HPLC with the following conditions: column: Xselect CSH C18 OBD, 30*150 mm 5 m, n; mobile phase A: water (0.1% FA), mobile phase B: ACN; flow rate 60 mL/min; gradient: 44% B to 55% B in 8 min; wavelength: 254; 220 nm; Rt1(min): 4.57, 6.43(min); injection volume: 1.5 mL; number of runs: 3) to afford N-(6-methoxy-1-methylindazol-7-yl)-6-[1-methyl-5-(trifluoromethyl)pyrazol-3-yl]pyridine-3-sulfonamide (7.9 mg, 2.57%) as an off-white solid. LCMS: (ES, m/z): [M+H]⁺ 467; H NMR (400 MHz, DMSO-d₆) δ 3.11 (s, 3H), 4.09 (s, 3H), 4.27 (s, 3H), 6.81 (d, J 8.8 Hz, 1H), 7.51 (s, 1H), 7.68 (d, J 8.8 Hz, 1H), 7.99 (s, 1H), 8.09 (dd, J 8.4, 2.4 Hz, 1H), 8.15 (d, J 8.4 Hz, 1H), 8.77 (d, J 2.4 Hz, 1H), 10.04 (s, 1H).

### Example 214—Synthesis of N-(1-Methyl-1H-Indol-7-yl)-6-(5-(Trifluoromethyl)-1H-Pyrazol-3-yl)Pyridine-3-Sulfonamide (I-143)

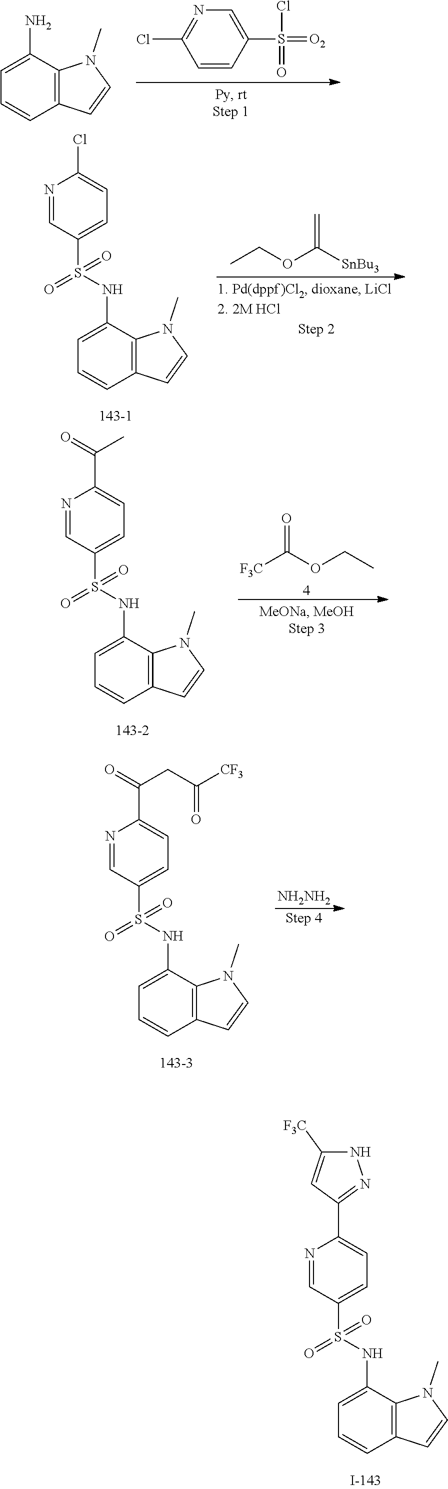

Step 1: Synthesis of 143-1. A solution of 1-methylindol-7-amine (292 mg, 1.997 mmol, 1 equiv.) and 6-chloropyridine-3-sulfonyl chloride (635.28 mg, 2.996 mmol, 1.5 equiv.) in pyridine (5 mL) was stirred overnight at room temperature. The resulting mixture was concentrated under reduced pressure and diluted with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (5 mmol/L NH₄HCO₃), 0% to 100% gradient in 20 min; detector: UV 254 nm, to give 6-chloro-N-(1-methylindol-7-yl)pyridine-3-sulfonamide (440 mg, 65.04%) as a red solid.

Step 2: Synthesis of 143-2. A solution of 6-chloro-N-(1-methylindazol-7-yl)pyridine-3-sulfonamide (323 mg, 1.001 mmol, 1 equiv.), Pd(PPh₃)₂Cl₂ (140.91 mg, 0.201 mmol, 0.2 equiv.), LiCl (85.10 mg, 2.008 mmol, 2 equiv.) and tributyl(1-ethoxyethenyl)stannane (725.05 mg, 2.008 mmol, 2 equiv.) in 1,4-dioxane (10 mL) was stirred for 3 h at 100° C. under a nitrogen atmosphere. The mixture was treated with 2 M HCl (1 mL) for 30 min. The reaction was quenched by the addition of KF (aq.) (20 mL) at room temperature. The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give 6-acetyl-N-(1-methylindol-7-yl)pyridine-3-sulfonamide (69 mg, 20.45%) as a brown solid.

Step 3: Synthesis of 143-3. A solution of 6-acetyl-N-(1-methylindazol-7-yl)pyridine-3-sulfonamide (330 mg, 0.999 mmol, 1 equiv.), MeONa (541.27 mg, 10.020 mmol, 10 equiv.) and trifluoroethyl acetate (284.70 mg, 2.004 mmol, 2 equiv.) in MeOH (5 mL) was stirred for 3 h at 70° C. The resulting mixture was diluted with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 20 min; detector: UV 254 nm, to give N-(1-methylindazol-7-yl)-6-(4,4,4-trifluoro-3-oxobutanoyl)pyridine-3-sulfonamide (410 mg, 94.34%) as a brown solid.

Step 4: Synthesis of N-(1-methyl-1H-indol-7-yl)-6-(5-(trifluoromethyl)-1H-pyrazol-3-yl)pyridine-3-sulfonamide (1-143). A solution of N-(1-methylindol-7-yl)-6-(4,4,4-trifluoro-3-oxobutanoyl)pyridine-3-sulfonamide (100 mg, 0.235 mmol, 1 equiv.) and NH₂NH₂. H₂O (58.84 mg, 1.175 mmol, 5 equiv.) in EtOH (3 mL) was stirred overnight at 50° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by prep-HPLC with the following conditions: column: Xselect CSH C18 OBD Column 30*150 mm 5 m; mobile phase A: water (0.1% FA), mobile phase B: ACN; flow rate 60 mL/min; gradient: 45% B to 55% B in 8 min; wavelength: 254; 220 nm; Rt1(min): 7.85; injection volume: 1.3 mL; number of runs: 2) to afford N-(1-methylindol-7-yl)-6-[5-(trifluoromethyl)-1H-pyrazol-3-yl]pyridine-3-sulfonamide (9.6 mg, 9.51%) as a white solid. LCMS: (ES, m/z): [M+H]⁺ 422; (400 MHz, DMSO-d₆) δ 4.08 (s, 3H), 6.22 (d, J7.2 Hz, 1H), 6.43 (d, J3.2 Hz, 1H), 6.75(d, J7.6 Hz, 1H), 7.28 (d, J 2.8 Hz, 1H), 7.41 (d, J 8.0 Hz, 1H), 7.56 (s, 1H), 8.15 (s, 1H), 8.18(d, J 1.2 Hz, 1H), 8.78 (s, 1H), 10.22 (s, 1H), 14.68 (s, 1H).

### Example 215—Synthesis of N-(6-Methoxy-1-Methyl-1H-Indazol-7-yl)-6-(5-Methyl-2H-Tetrazol-2-yl)Pyridine-3-Sulfonamide (I-144)

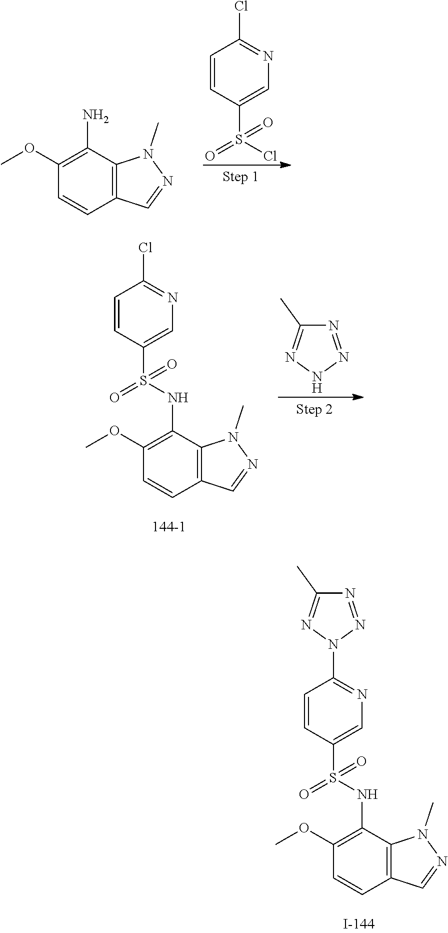

Step 1: Synthesis of 144-1. To a stirred solution of 6-methoxy-1-methylindazol-7-amine (2 g, 11.286 mmol, 1 equiv.) and 6-chloropyridine-3-sulfonyl chloride (2.63 g, 12.415 mmol, 1.1 equiv.) in MeCN (6 mL) was added pyridine (4.80 mL, 59.703 mmol, 5.29 equiv.) dropwise at room temperature under a nitrogen atmosphere. The resulting mixture was stirred for an additional 2 h at room temperature. The resulting mixture was diluted with water (10 mL). The resulting mixture was extracted with EtOAc (3×10 mL). The combined organic layers were washed with brine (2×10 mL) and dried over anhydrous Na₂SO₄. After filtration, the filtrate was concentrated under reduced pressure. This resulted in 6-chloro-N-(6-methoxy-1-methylindazol-7-yl) pyridine-3-sulfonamide (3 g, 60.28%) as a light yellow solid.

Step 2: Synthesis of N-(6-methoxy-1-methyl-1H-indazol-7-yl)-6-(5-methyl-2H-tetrazol-2-yl)pyridine-3-sulfonamide (1-144). To a stirred solution of 6-chloro-N-(6-methoxy-1-methylindazol-7-yl)pyridine-3-sulfonamide (200 mg, 0.567 mmol, 1 equiv.) and t-BuOK (190.84 mg, 1.701 mmol, 3 equiv.) in DMSO (5 mL) was added 5-methyl-2H-1,2,3,4-tetrazole (71.50 mg, 0.850 mmol, 1.5 equiv.) dropwise at room temperature under a nitrogen atmosphere. The resulting mixture was stirred overnight at 140° C. The resulting mixture was diluted with water (10 mL). The resulting mixture was extracted with EtOAc (3×10 mL). The combined organic layers were washed with brine (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 80% gradient in 20 min; detector: UV 254 nm. The crude product (60 mg) was purified by prep-HPLC with the following conditions: column: GreenSep Naphthyl, 4.6*100 mm, 3 um; mobile phase B: MeOH (1% 2M NH₃-MeOH); flow rate 4 mL/min; gradient: isocratic 10% B; wavelength: 220 nm) to afford N-(6-methoxy-1-methylindazol-7-yl)-6-(5-methyl-1,2,3,4-tetrazol-2-yl)pyridine-3-sulfonamide (35.6 mg, 15.64%) as an off-white solid. LCMS: (ES, m/z): [M+H]⁺ 401; ¹H NMR (400 MHz, DMSO-d₆) δ 2.85 (s, 3H), 3.21 (s, 3H), 4.28 (s, 3H), 6.84 (d, J 8.8 Hz, 1H), 7.68 (d, J 8.8 Hz, 1H), 8.00 (s, 1H), 8.21 (dd, J 8.4, 0.8 Hz, 1H), 8.38 (dd, J 8.4, 2.4 Hz, 1H), 8.83 (dd, J 2.4, 0.8 Hz, 1H), 10.23 (s, 1H).

### Example 216—Synthesis of N-(1-Methyl-1H-Indazol-7-yl)-[2,3′-Bipyridine]-5-Sulfonamide (1-145)

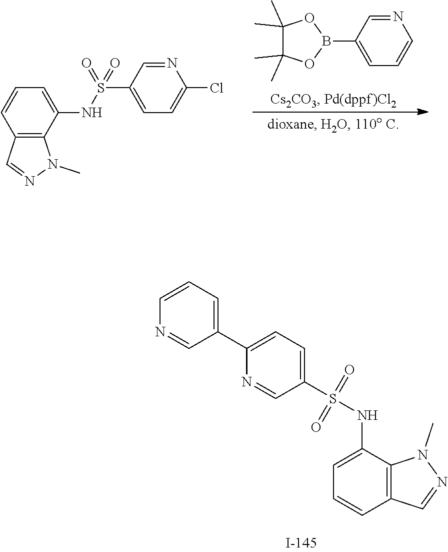 [SMILES: Cn1ncc2cccc(NS(=O)(=O)c3ccc(Cl)nc3)c21 ; CC1(C)OB(c2cccnc2)OC1(C)C ; Cn1ncc2cccc(NS(=O)(=O)c3ccc(-c4cccnc4)nc3)c21]

To a stirred solution of 6-chloro-N-(1-methylindazol-7-yl) pyridine-3-sulfonamide (100 mg, 0.310 mmol, 1 equiv.) and 2-(4,4,5,5-tetramethyl-1,3,2-dioxaborolan-2-yl)pyridine (63.53 mg, 0.310 mmol, 1 equiv.) in H₂O (1 mL) and 1,4-dioxane (4 mL) were added Cs₂CO₃ (302.83 mg, 0.930 mmol, 3 equiv.) and Pd(dppf)Cl₂ (45.34 mg, 0.062 mmol, 0.2 equiv.) under a nitrogen atmosphere. The resulting mixture was stirred overnight at 110° C. under a nitrogen atmosphere. The resulting mixture was diluted with water (10 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in N-(1-methylindazol-7-yl)-[2,2′-bipyridine]-5-sulfonamide (2.6 mg, 2.25%) as a white solid. LCMS: (ES, m/z): [M+H]⁺ 366; (400 MHz, DMSO-d₆) δ 4.30 (s, 3H), 6.53 (d, J 7.2 Hz, 1H), 6.92 (t, J 7.8 Hz, 1H), 7.67 (d, J 8.4 Hz, 2H), 8.09 (t, J 8.4 Hz, 2H), 8.21 (dd, J 8.4, 2.4 Hz, 1H), 8.44 (d, J 8.4 Hz, 1H), 8.57 (d, J 8.4 Hz, 1H), 8.75 (d, J 4.4 Hz,1H), 8.87 (d, J 4.4 Hz, 1H), 10.42 (s, 1H).

### Example 217—Synthesis of 6-(1-(2-Hydroxyethyl)-1H-Pyrazol-4-yl)-N-(1-Methyl-1H-Indazol-7-yl)Pyridine-3-Sulfonamide (I-146)

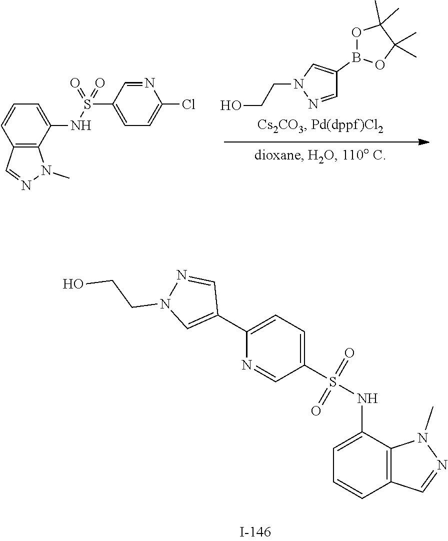 [SMILES: Cn1ncc2cccc(NS(=O)(=O)c3ccc(Cl)nc3)c21 ; CC1(C)OB(c2cnn(CCO)c2)OC1(C)C ; Cn1ncc2cccc(NS(=O)(=O)c3ccc(-c4cnn(CCO)c4)nc3)c21]

To a stirred solution of 6-chloro-N-(1-methylindazol-7-yl)pyridine-3-sulfonamide (100 mg, 0.310 mmol, 1 equiv.) and 2-[4-(4,4,5,5-tetramethyl-1,3,2-dioxaborolan-2-yl)pyrazol-1-yl]ethanol (73.76 mg, 0.310 mmol, 1 equiv.) in H₂O (1 mL) and 1,4-dioxane (4 mL) were added Cs₂CO₃ (302.83 mg, 0.930 mmol, 3 equiv.) and Pd(dppf)Cl₂ (45.34 mg, 0.062 mmol, 0.2 equiv.) under a nitrogen atmosphere. The resulting mixture was stirred overnight at 110° C. under a nitrogen atmosphere. The resulting mixture was diluted with water (10 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 6-[1-(2-hydroxyethyl)pyrazol-4-yl]-N-(1-methylindazol-7-yl)pyridine-3-sulfonamide (49.2 mg, 39.54%) as a white solid. LCMS: (ES, m/z): [M+H]⁺ 399; (400 MHz, DMSO-d₆) δ 3.78 (q, J 5.6 Hz, 2H), 4.21 (t, J 5.6 Hz, 2H), 4.27 (s, 3H), 4.95 (t, J 5.2 Hz, 1H), 6.49 (d, J 7.2 Hz, 1H), 6.94 (t, J 7.6 Hz, 1H), 7.69 (d, J 8.0 Hz, 1H), 7.87 (d, J 8.4 Hz, 1H), 7.96 (dd, J 8.4, 2.4 Hz, 1H), 8.09 (s, 1H), 8.14 (s, 1H), 8.44 (s, 1H), 8.65 (d, J 2.4 Hz, 1H), 10.17 (s, 1H).

### Example 218—Synthesis of 6-(5-Cyclopropyl-1H-Pyrazol-1-yl)-N-(6-Methoxy-1-Methyl-1H-Indazol-7-yl)Pyridine-3-Sulfonamide (I-147)

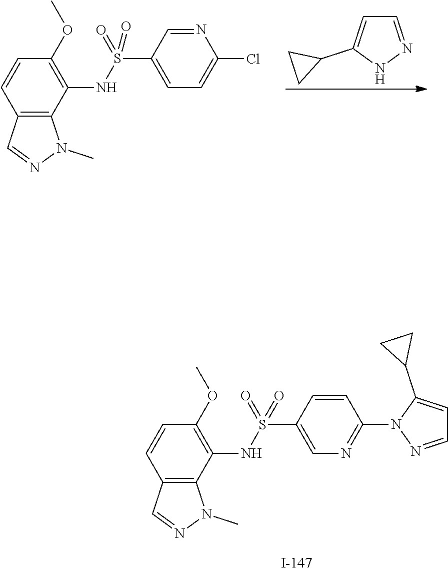

A mixture of 6-chloro-N-(5-methoxy-3-methyl-1,2-dihydro-1,2,3-benzotriazol-4-yl) pyridine-3-sulfonamide (100 mg, 0.281 mmol, 1.00 equiv.), 3-cyclopropyl-2H-pyrazole (33 mg, 0.311 mmol, 1.10 equiv.), CuI (53 mg, 0.283 mmol, 1.00 equiv.) and Cs₂CO₃ (277 mg, 0.849 mmol, 3.00 equiv.) in DMF (2 mL) was stirred overnight at 140° C. under a nitrogen atmosphere. The reaction was quenched with water at room temperature. The resulting mixture was extracted with EtOAc (3×3 mL). The combined organic layers were washed with brine (5 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃), mobile phase B: ACN; flow rate 60 mL/min; gradient: 35% B to 61% B in 8 min; wavelength: 254 nm) to afford 6-(5-cyclopropylpyrazol-1-yl)-N-(5-methoxy-3-methyl-1,2-dihydro-1,2,3-benzotriazol-4-yl)pyridine-3-sulfonamide (2.1 mg, 1.75%) as a white solid. LCMS (ESI, m/z): [M+H]⁺ 425; ¹H NMR (400 MHz, DMSO-d₆) δ 0.69-0.72 (m, 2H), 0.97-1.00 (m, 2H), 2.79-2.83 (m, 1H), 4.28 (s, 3H), 6.23 (s, 1H), 6.84 (d, J 8.8 Hz, 1H), 7.66-7.70 (m, 2H), 7.99-8.04 (m, 2H), 8.18 (dd, J 8.8, 2.4 Hz, 1H), 8.64 (d, J2.4 Hz, 1H), 10.01 (s, 1H).

### Example 219—Synthesis of 6-(4,5-Dimethyl-1H-Pyrazol-1-yl)-N-(6-Methoxy-1-Methyl-1H-Indazol-7-yl)Pyridine-3-Sulfonamide (I-148)

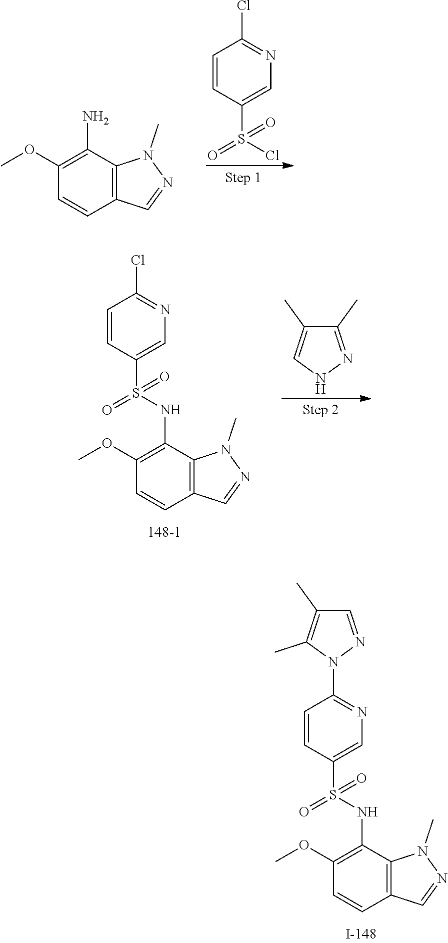

Step 1: Synthesis of 148-1. To a stirred solution of 6-methoxy-1-methylindazol-7-amine (2 g, 11.286 mmol, 1 equiv.) and 6-chloropyridine-3-sulfonyl chloride (2.63 g, 12.415 mmol, 1.1 equiv.) in MeCN (6 mL) was added pyridine (4.80 mL, 59.703 mmol, 5.29 equiv.) dropwise at room temperature under a nitrogen atmosphere. The resulting mixture was stirred for an additional 2 h at room temperature. The resulting mixture was diluted with water (10 mL). The resulting mixture was extracted with EtOAc (3×10 mL). The combined organic layers were washed with brine (2×10 mL) and dried over anhydrous Na₂SO₄. After filtration, the filtrate was concentrated under reduced pressure. This resulted in 6-chloro-N-(6-methoxy-1-methylindazol-7-yl) pyridine-3-sulfonamide (3 g, 60.28%) as a light yellow solid.

Step 2: Synthesis of 6-(4,5-dimethyl-1H-pyrazol-1-yl)-N-(6-methoxy-1-methyl-11H-indazol-7-yl)pyridine-3-sulfonamide (1-148). To a stirred solution of 6-chloro-N-(6-methoxy-1-methylindazol-7-yl) pyridine-3-sulfonamide (200 mg, 0.567 mmol, 1 equiv.) and t-BuOK (190.84 mg, 1.701 mmol, 3 equiv.) in DMSO (4 mL) were added 3,4-dimethyl-1H-pyrazole (109.00 mg, 1.134 mmol, 2 equiv.) dropwise at room temperature under a nitrogen atmosphere. The resulting mixture was stirred overnight at 140° C. The resulting mixture was diluted with water (10 mL). The resulting mixture was extracted with EtOAc (3×10 mL). The combined organic layers were washed with brine (3×10 mL) and dried over anhydrous Na₂SO₄. The residue was purified by reversed-phase flash chromatography with the following conditions: column: C18 silica gel; mobile phase: MeCN in water (0.1% TFA), 20% to 80% gradient in 20 min; detector: UV 254 nm. The crude product (170 mg) was purified by prep-HPLC with the following conditions: column: XBridge Prep OBD C18, 30*150 mm, 5 m; mobile phase A: water (10 mmol/L NH₄HCO₃+0.1% NH₃·H₂O), mobile phase B: ACN; flow rate 60 mL/min; gradient: 25% B to 51% B in 11 min, 51% B; wavelength: 220 nm; Rt1(min): 10.53) to afford 6-(4,5-dimethylpyrazol-1-yl)-N-(6-methoxy-1-methylindazol-7-yl) pyridine-3-sulfonamide (6.5 mg, 2.77%) as an off-white solid. LCMS: (ES, m/z): [M+H]⁺ 413; ¹H NMR (400 MHz, DMSO-d₆) δ 2.03 (s, 3H), 2.57 (s, 3H), 3.19 (s, 3H), 4.27 (s, 3H), 6.83 (d, J 8.8 Hz, 1H), 7.63 (s, 1H), 7.66 (d, J 8.8 Hz, 1H), 7.94 (s, 1H), 8.01 (d, J 8.0 Hz, 1H), 8.14 (dd, J 8.8, 2.5 Hz, 1H), 8.60 (d, J 2.4 Hz, 1H), 9.99 (s, 1H).

### Example 220—Synthesis of N-(6-Methoxy-1-Methyl-1H-Indazol-7-yl)-6-(2-Methylthiazol-5-yl)Pyridine-3-Sulfonamide (1-149)

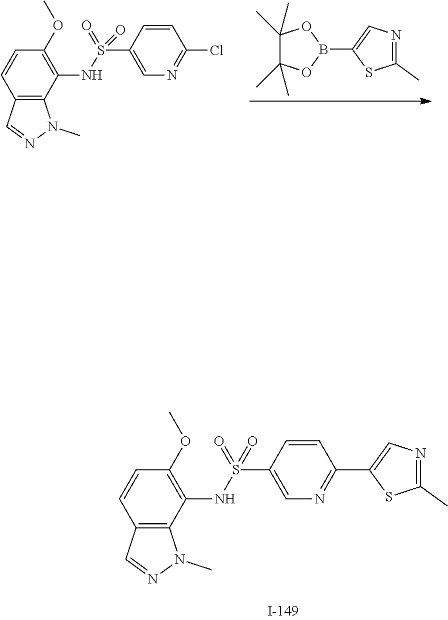

A solution of 6-chloro-N-(6-methoxy-1-methyl-1H-indazol-7-yl)pyridine-3-sulfonamide (100 mg, 0.283 mmol, 1.00 equiv.), 2-methyl-5-(4,4,5,5-tetramethyl-1,3,2-dioxaborolan-2-yl)thiazole (127.62 mg, 0.566 mmol, 2.00 equiv.), Pd(dppf)Cl₂CH₂Cl₂ (46.18 mg, 0.057 mmol, 0.20 equiv.) and Cs₂CO₃ (277.06 mg, 0.849 mmol, 3.00 equiv.) in dioxane (4 mL) and H₂O (1 mL) was stirred for 2 h at 90° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: Column: XBridge Prep OBD C18, 30*150 mm, 5 m; mobile phase A: water (10 mmol NH₄HCO₃+0.05% NH₃H₂O), mobile phase B: ACN; flow rate 60 mL/min; gradient: 16% B to 41% B in 8 min; wavelength: 220 nm; Rt1(min): 7.98, to afford N-(6-methoxy-1-methyl-1H-indazol-7-yl)-6-(2-methylthiazol-5-yl)pyridine-3-sulfonamide (15.9 mg, 13.31%) as a light yellow solid. LCMS: (ES, m/z): [M+H]⁺416; (400 MHz, DMSO-d₆) δ 2.71 (s, 3H), 3.13 (s, 3H), 4.27 (s, 3H), 6.81 (d, J 8.8 Hz, 1H), 7.66 (d, J 8.8 Hz, 1H), 7.96 (s, 1H), 8.07 (d, J 8.8 Hz, 1H), 8.15 (d, J 8.8 Hz, 1H), 8.49 (s, 1H), 8.69 (d, J 2.4 Hz, 1H), 9.97 (s, 1H).

### Example 221—Synthesis of 6-(4-Fluoro-1H-Pyrazol-1-yl)-N-(6-Methoxy-1-Methyl-1H-Indazol-7-yl)Pyridine-3-Sulfonamide (I-150)

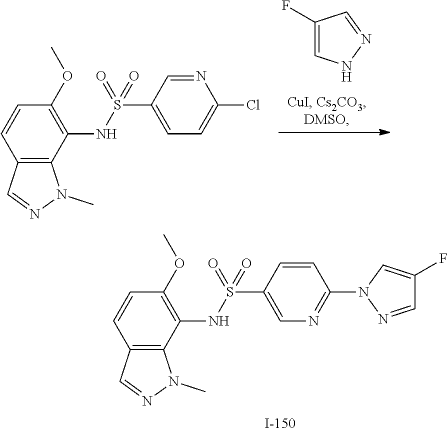 [SMILES: COc1ccc2cnn(C)c2c1NS(=O)(=O)c1ccc(Cl)nc1 ; Fc1cn[nH]c1 ; COc1ccc2cnn(C)c2c1NS(=O)(=O)c1ccc(-n2cc(F)cn2)nc1]

To a stirred mixture of 6-chloro-N-(6-methoxy-1-methylindazol-7-yl)pyridine-3-sulfonamide (100 mg, 0.283 mmol, 1 equiv.) and (1R,2R)—N1,N2-dimethylcyclohexane-1,2-diamine (20.16 mg, 0.141 mmol, 0.5 equiv.) in DMSO (2.5 mL) were added 4-fluoro-1H-pyrazole (29.28 mg, 0.340 mmol, 1.2 equiv.), CuI (10.80 mg, 0.057 mmol, 0.2 equiv.) and Cs₂CO₃ (277.06 mg, 0.849 mmol, 3 equiv.) in portions at room temperature under a nitrogen atmosphere. The resulting mixture was stirred overnight at 140° C. under a nitrogen atmosphere. The aqueous layer was extracted with EtOAc (3×5 mL). The combined organic layers were washed with brine (2×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 45% to 55% gradient in 10 min; detector: UV 254 nm. The resulting mixture was concentrated under reduced pressure. This resulted in 6-(4-fluoropyrazol-1-yl)-N-(6-methoxy-1-methylindazol-7-yl)pyridine-3-sulfonamide (3.3 mg, 2.79%) as a white solid. LCMS: (ES, m/z): [M+H]⁺ 403; ¹H NMR (400 MHz, DMSO-d₆): 3.18 (s, 3H), 4.27 (s, 3H), 6.82 (d, J8.8 Hz, 1H), 7.65 (d, J8.8 Hz, 1H), 7.98 (s, 1H), 8.05-8.12 (m, 2H), 8.21 (dd, J8.4, 2.4 Hz, 1H), 8.61 (d, J2.0 Hz, 1H), 8.82 (d, J4.0 Hz, 1H), 10.03 (s, 1H).

### Example 222—Synthesis of N-(6-Methoxy-1-Methyl-1H-Indazol-7-yl)-6-(5-Methoxy-1H-Pyrazol-1-yl)Pyridine-3-Sulfonamide (1-151)

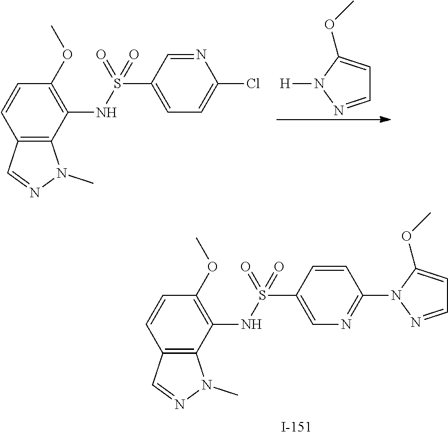

A solution of 6-chloro-N-(6-methoxy-1-methyl-1H-indazol-7-yl)pyridine-3-sulfonamide (100 mg, 0.283 mmol, 1.00 equiv.), 5-methoxy-1H-pyrazole (41.7 mg, 0.424 mmol, 1.50 equiv.), CuI (10.8 mg, 0.057 mmol, 0.20 equiv.), N1,N2-dimethylcyclohexane-1,2-diamine (10.0 mg, 0.071 mmol, 0.25 equiv.) and Cs₂CO₃ (277.06 mg, 0.849 mmol, 3.00 equiv.) in DMSO (2 mL) was stirred for 2 h at 90° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Shield RP18 OBD, 30*150 mm, 5 μm; mobile phase A: water (10 mmol/L NH₄HCO₃+0.1% NH₃. H₂O), mobile phase B: ACN; flow rate 60 mL/min; gradient: 30% B to 55% B in 8 min; wavelength: 220/254 nm; Rt1(min): 7.5, to afford N-(6-methoxy-1-methyl-1H-indazol-7-yl)-6-(5-methoxy-1H-pyrazol-1-yl)pyridine-3-sulfonamide (20.9 mg, 17.31%) as a light yellow solid. LCMS: (ES, m/z): [M+H]⁺ 415; ¹H NMR (400 MHz, DMSO-d₆) δ 3.19 (s, 3H), 3.94 (s, 3H), 4.27 (s, 3H), 6.20 (d, J 2.8 Hz, 1H), 6.83 (d, J 8.8 Hz, 1H), 7.67 (d, J 8.8 Hz, 1H), 7.87 (d, J 8.4 Hz, 1H), 7.99 (s, 1H), 8.13 (dd, J 8.8 Hz, 2.4 Hz, 1H), 8.54 (d, J 2.4 Hz, 1H), 8.55 (d, J 2.8 Hz, 1H), 10.0(s, 1H).

### Example 223—Synthesis of N-(1-Methyl-1H-Indazol-7-yl)-6-(4-Propionyl-1H-Pyrazol-1-yl)Pyridine-3-Sulfonamide (I-152)

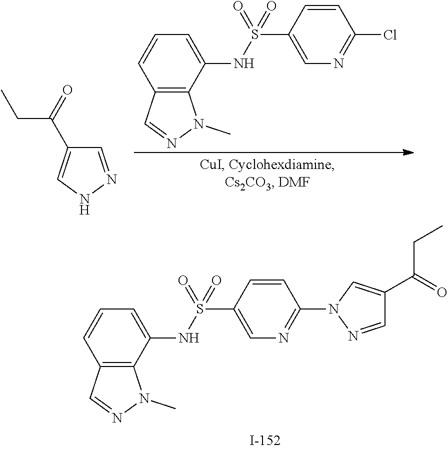

A mixture of 1-(1H-pyrazol-4-yl)cyclopropan-1-ol (160.5 mg, 1.293 mmol, 1 equiv.), Cs₂CO₃ (839.85 mg, 2.578 mmol, 2 equiv.), CuI (122.73 mg, 0.644 mmol, 0.5 equiv.) and cyclohexane-1,4-diamine (147.17 mg, 1.289 mmol, 1 equiv.) in DMF (8 mL) was stirred overnight at 140° C. under a nitrogen atmosphere. The resulting mixture was diluted with water (100 mL) and then extracted with EtOAc (4×100 mL). The combined organic layers were washed with brine (100 mL) and dried over anhydrous Na₂SO₄. After filtration, the filtrate was concentrated under reduced pressure to give a residue. The residue was purified by reversed-phase flash chromatography with the following conditions: column: C18 silica gel; mobile phase: ACN in water, 0% to 100% gradient in 10 min; detector: UV 254 nm to give the crude product, and the crude product was purified by prep-HPLC with the following conditions: column: Xselect CSH C₁₈ OBD, 30*150 mm 5 m, n; mobile phase A: water (0.1% FA), mobile phase B: ACN; flow rate 60 mL/min; gradient: 33% B to 47% B in 8 min, 47% B; wavelength: 254; 220 nm; Rt1(min): 7.65; injection volume: 1.5 mL) to afford N-(1-methyl-1H-indazol-7-yl)-6-(4-propionyl-1H-pyrazol-1-yl)pyridine-3-sulfonamide (15.1 mg, 2.62%) as an off-white solid. LCMS: (ES, m/z): [M+H]⁺ 411; ¹H NMR (400 MHz, DMSO-d₆) δ 1.07 (t, J7.2 Hz, 3H), 2.96 (q, J7.2 Hz, 2H), 4.30 (s, 3H), 6.53 (d, J=7.2 Hz, 1H), 6.92 (t, J8.0 Hz, 1H), 7.67 (d, J 8.0 Hz,1H), 8.09 (s, 1H), 8.18 (d, J8.8 Hz,1H), 8.28-8.33 (m, 2H), 8.67 (d, J2.0 Hz,1H), 9.32 (s, 1H), 10.41 (s, 1H).

### Example 224—Synthesis of 6-(3-Cyclopropyl-1H-Pyrazol-1-yl)-N-(6-Methoxy-1-Methyl-1H-Indazol-7-yl)Pyridine-3-Sulfonamide (I-153)

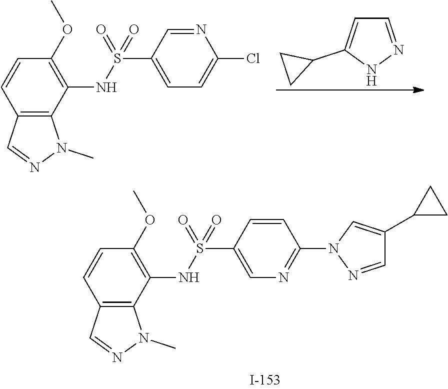

A mixture of 6-chloro-N-(5-methoxy-3-methyl-1,2-dihydro-1,2,3-benzotriazol-4-yl) pyridine-3-sulfonamide (100 mg, 0.281 mmol, 1 equiv.), 3-cyclopropyl-2H-pyrazole (33.72 mg, 0.311 mmol, 1.1 equiv.), CuI (53.98 mg, 0.283 mmol, 1 equiv.) and Cs₂CO₃ (277.06 mg, 0.849 mmol, 3 equiv.) in DMF (2 mL) was stirred overnight at 140° C. under a nitrogen atmosphere. The reaction was quenched with water at room temperature. The resulting mixture was extracted with EtOAc (3×3 mL). The combined organic layers were washed with brine (5 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water(10 mmol/L NH₄HCO₃), mobile phase B: ACN; flow rate 60 mL/min; gradient: 35% B to 61% B in 8 min; wavelength: 254 nm) to afford 6-(3-cyclopropylpyrazol-1-yl)-N-(6-methoxy-1-methylindazol-7-yl)pyridine-3-sulfonamide (15.4 mg, 12%) as a white solid. LCMS (ESI, m/z): [M+H]⁺ 383; ¹H NMR (400 MHz, DMSO-d₆) δ 0.79-0.83 (m, 2H), 0.97-1.00 (m, 2H), 2.00-2.05 (m, 1H), 3.19 (s, 3H), 4.27 (s, 3H), 6.37 (d, J2.8 Hz, 1H), 6.83 (d, J8.8 Hz, 1H), 7.67 (d, J8.8 Hz, 1H), 7.98-8.00 (m, 2H), 8.14 (dd, J8.8, 2.4 Hz, 1H), 8.54-8.56 (m, 2H), 9.98 (s, 1H).

### Example 225—Synthesis of N-(1-Methyl-1H-Indazol-7-yl)-6-(3-(Trifluoromethyl)-1H-Pyrazol-5-yl)Pyridine-3-Sulfonamide (I-84)

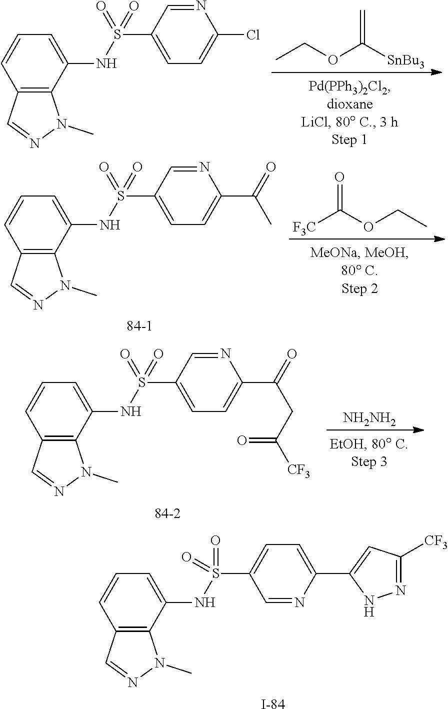

Step 1: Synthesis of 84-1. A solution of 6-chloro-N-(1-methylindazol-7-yl)pyridine-3-sulfonamide (300 mg, 0.929 mmol, 1 equiv.) tributyl(1-ethoxyethenyl)stannane (503.52 mg, 1.393 mmol, 1.5 equiv.), Pd(PPh₃)₂Cl₂ (24.38 mg, 0.093 mmol, 0.1 equiv.) and LiCl (78.80 mg, 1.858 mmol, 2 equiv.) in dioxane (5 mL) was stirred for 3 h at 80° C. under a nitrogen atmosphere. The mixture was neutralized to pH 7 with HCl (aq.). The resulting mixture was diluted with water (5 mL). The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in 84-1 (200 mg, 65.14%) as a yellow oil.

Step 2: Synthesis of 84-2. A solution of 84-1 and MeONa (58.87 mg, 1.090 mmol, 2 equiv.) in MeOH (5 mL) was stirred for 3 h at 80° C. under a nitrogen atmosphere. The residue was diluted with water (5 mL). The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in 84-2 (125 mg, 53.81%) as an off-white solid.

Step 3: Synthesis of N-(1-methyl-1H-indazol-7-yl)-6-(3-(trifluoromethyl)-1H-pyrazol-5-yl)pyridine-3-sulfonamide (1-84). A solution of 84-2 (100 mg, 0.235 mmol, 1 equiv.) and hydrazine (15.03 mg, 0.470 mmol, 2 equiv.) in EtOH (2 mL) was stirred for 4 h at room temperature under a nitrogen atmosphere. The residue was dissolved in water (5 mL). The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₃·H₂O), 40% to 50% gradient in 10 min; detector: UV 254 nm. This resulted in N-(1-methyl-1H-indazol-7-yl)-6-(3-(trifluoromethyl)-1H-pyrazol-5-yl)pyridine-3-sulfonamide (20.9 mg, 20.78%) as an off-white solid. LCMS: (ES, m/z): [M+H]⁺ 423; (400 MHz, DMSO-d₆): δ 4.30 (s, 3H), 6.54 (d, J7.2 Hz, 1H), 6.89 (t, J7.6 Hz, 1H), 7.55 (s, 1H), 8.04 (s, 1H), 8.18 (dd, J8.0, 2.0 Hz, 2H), 8.80 (s, 1H), 10.35 (s, 1H), 14.62 (s, 1H).

### Example 226—Synthesis of N-(6-Methoxy-1-Methyl-1H-Indazol-7-yl)-N-Methyl-6-(1-Methyl-3-(Trifluoromethyl)-1H-Pyrazol-5-yl)Pyridine-3-Sulfonamide (1-104)

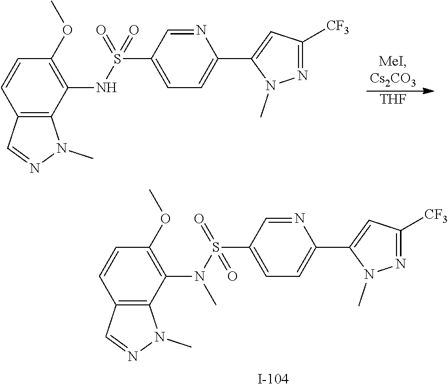 [SMILES: COc1ccc2cnn(C)c2c1NS(=O)(=O)c1ccc(-c2cc(C(F)(F)F)nn2C)nc1 ; COc1ccc2cnn(C)c2c1N(C)S(=O)(=O)c1ccc(-c2cc(C(F)(F)F)nn2C)nc1 ; "THF"]

To a stirred solution of N-(6-methoxy-1-methylindazol-7-yl)-6-[2-methyl-5-(trifluoromethyl)pyrazol-3-yl]pyridine-3-sulfonamide (35 mg, 0.075 mmol, 1 equiv.) and Cs₂CO₃ (73.34 mg, 0.225 mmol, 3 equiv.) in THE (2 mL) was added methyl iodide (21.30 mg, 0.150 mmol, 2 equiv.) dropwise at room temperature under a nitrogen atmosphere. The resulting mixture was stirred for 2 h at room temperature under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep Phenyl OBD, 19*250 mm, 5 m; mobile phase A: water (10 mmol/L NH₄HCO₃+0.1% NH₃·H₂O), mobile phase B: ACN; flow rate 25 mL/min; gradient: 37% B to 62% B in 11 min; wavelength: 220/254 nm; Rt1(min): 10.45; injection volume: 0.4 mL; number of runs: 4) to afford N-(6-methoxy-1-methylindazol-7-yl)-N-methyl-6-[2-methyl-5-(trifluoromethyl)pyrazol-3-yl] pyridine-3-sulfonamide (8.4 mg, 23.18%) as a white solid. LCMS: (ES, m/z): [M+H]⁺ 481; ¹H NMR (400 MHz, DMSO-d₆) δ 3.18 (s, 3H), 3.33 (s, 3H), 4.11 (s, 3H), 4.24 (s, 3H), 6.89 (d, J 8.8 Hz, 1H), 7.53 (s, 1H), 7.76 (d, J 8.8 Hz, 1H), 8.02 (s, 1H), 8.15 (d, J 8.3 Hz, 1H), 8.24 (dd, J 8.4, 2.4 Hz, 1H), 8.82 (dd, J 2.4, 1.2 Hz, 1H).

### Example 227—Synthesis of N-(1-Methyl-1H-Indazol-7-yl)-2′-Oxo-1′,2′-Dihydro-[2,4′-Bipyridine]-5-Sulfonamide (I-154)

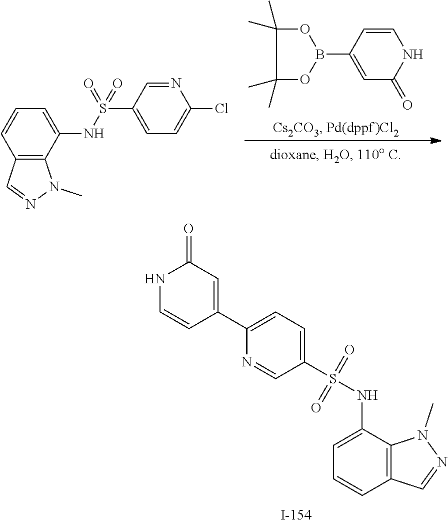

To a stirred mixture of 6-chloro-N-(1-methylindazol-7-yl)pyridine-3-sulfonamide (100 mg, 0.310 mmol, 1 equiv.) and 4-(4,4,5,5-tetramethyl-1,3,2-dioxaborolan-2-yl)-1H-pyridin-2-one (68.49 mg, 0.310 mmol, 1 equiv.) in dioxane (4 mL) and H₂O (1 mL) were added Pd(dppf)Cl₂ (45.34 mg, 0.062 mmol, 0.2 equiv.) and Cs₂CO₃ (70.76 mg, 0.930 mmol, 3 equiv.) in portions at room temperature under a nitrogen atmosphere. The resulting mixture was stirred overnight at 110° C. under a nitrogen atmosphere. The resulting mixture was diluted with water (10 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. The resulting mixture was concentrated under vacuum. This resulted in N-(1-methylindazol-7-yl)-2′-oxo-1′H-[2,4′-bipyridine]-5-sulfonamide (30.7 mg, 25.75%) as a white solid. LCMS: (ES, m/z): [M+H]⁺ 382; (400 MHz, DMSO-d₆) δ 4.28 (s, 3H), 6.51 (d, J 7.2 Hz, 1H), 6.91 (q, J 7.2, 6.4 Hz, 2H), 7.09 (s, 1H), 7.53 (d, J 6.8 Hz, 1H), 7.66 (s, 1H), 8.08 (s, 1H), 8.13 (dd, J 8.4, 2.4 Hz, 1H), 8.25 (d, J 8.4 Hz, 1H), 8.88 (d, J 2.4 Hz, 1H), 10.38 (s, 1H), 11.80 (s, 1H).

### Example 228—Synthesis of N-(1-Methyl-1H-Indazol-7-yl)-6-(5-Methyl-1H-Pyrazol-4-yl)Pyridine-3-Sulfonamide (I-155)

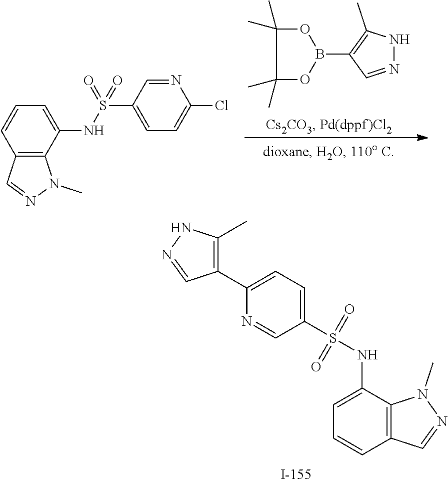

To a stirred solution of 6-chloro-N-(1-methylindazol-7-yl)pyridine-3-sulfonamide (100 mg, 0.310 mmol, 1 equiv.) and 3-methyl-4-(4,4,5-trimethyl-1,3,2-dioxaborolan-2-yl)-2H-pyrazole (60.12 mg, 0.310 mmol, 1 equiv.) in H₂O (1 mL) and 1,4-dioxane (4 mL) were added Pd(dppf)Cl₂ (45.34 mg, 0.062 mmol, 0.2 equiv.) and Cs₂CO₃ (70.76 mg, 0.930 mmol, 3 equiv.) under a nitrogen atmosphere. The resulting mixture was stirred overnight at 110° C. under a nitrogen atmosphere. The resulting mixture was diluted with water (10 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The resulting mixture was concentrated under vacuum. This resulted in 6-(3-methyl-2H-pyrazol-4-yl)-N-(1-methylindazol-7-yl)pyridine-3-sulfonamide (71.4 mg, 62.30%) as a white solid. LCMS: (ES, m/z): [M+H]⁺ 369; (400 MHz, DMSO-d₆) δ 2.54 (s, 3H), 4.28 (s, 3H), 6.52 (d, J 7.2 Hz, 1H), 6.93 (t, J 7.6 Hz, 1H), 7.67 (s, 1H), 7.83 (d, J 8.4 Hz, 1H), 7.94 (dd, J 8.4, 2.4 Hz, 1H), 8.08 (s, 2H), 8.69 (d, J 2.4 Hz, 1H), 10.16 (s, 1H), 12.98 (s, 1H).

### Example 229—Synthesis of N-(6-Methoxy-1-Methyl-1H-Pyrazolo[4,3-C]Pyridin-7-yl)-6-(3-(Trifluoromethyl)-1H-Pyrazol-1-yl)Pyridine-3-Sulfonamide (1-156)

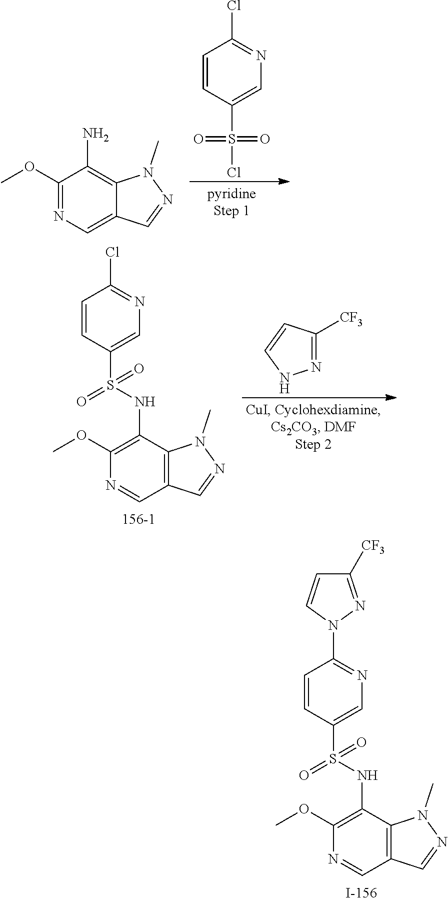

Step 1: Synthesis of 156-1. A solution of 6-methoxy-1-methylpyrazolo[4,3-c]pyridin-7-amine (178 mg, 0.999 mmol, 1 equiv.) and 6-chloropyridine-3-sulfonyl chloride (254.17 mg, 1.199 mmol, 1.2 equiv.) in pyridine (1 mL) was stirred overnight at room temperature. After the reaction was complete, the solvent was removed to give the crude product. The crude product was purified by reversed-phase flash chromatography with the following conditions: column: C18 silica gel; mobile phase: ACN in water, 0% to 100% gradient in 20 min; detector: UV 254 nm to give 156-1 (82 mg, 23.20%) as a yellow solid.

Step 2: Synthesis of N-(6-methoxy-1-methyl-1H-pyrazolo[4,3-c]pyridin-7-yl)-6-(3-(trifluoromethyl)-1H-pyrazol-1-yl)pyridine-3-sulfonamide (1-156). A mixture of 156-1 (82 mg, 0.232 mmol, 1 equiv.), 3-(trifluoromethyl)-1H-pyrazole (37.85 mg, 0.278 mmol, 1.2 equiv.), CuI (22.07 mg, 0.116 mmol, 0.5 equiv.), cyclohexane-1,4-diamine (26.47 mg, 0.232 mmol, 1 equiv.) and Cs₂CO₃ (151.04 mg, 0.464 mmol, 2 equiv.) in DMF (5 mL) was stirred overnight at 140° C. under a nitrogen atmosphere. After the reaction was completed, the resulting mixture was diluted with water (50 mL). The resulting mixture was extracted with EtOAc (4×40 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water, 0% to 100% gradient in 25 min; detector: UV 254 nm to give the crude. The crude was purified by prep-HPLC with the following conditions: column: Xselect CSH C₁₈ OBD, 30*150 mm 5 μm, n; mobile phase A: water (0.1% FA), mobile phase B: ACN; flow rate 60 mL/min; gradient: 37% B to 47% B in 8 min; wavelength: 254; 220 nm; Rt1(min): 7.65; injection volume: 1.3 mL; Number of Runs: 4) to afford N-(6-methoxy-1-methyl-1H-pyrazolo[4,3-c]pyridin-7-yl)-6-(3-(trifluoromethyl)-1H-pyrazol-1-yl)pyridine-3-sulfonamide (10.9 mg, 10.37%) as an off-white solid. LCMS: (ES, m/z): [M+H]⁺ 454; ¹H NMR (400 MHz, DMSO-d₆) δ 3.28 (s, 3H), 4.25 (s, 3H), 7.16 (d, J2.8 Hz, 1H), 8.18 (d, J8.4 Hz, 1H), 8.24 (s, 1H), 8.30 (dd, J8.8, 2.4 Hz, 1H), 8.65 (s, 1H), 8.70 (d, J2.0 Hz, 1H), 8.94 (d, J 1.6 Hz, 1H), 10.28 (s, 1H)

### Example 230—Synthesis of N-(6-Methoxy-1-Methyl-1H-Indazol-7-yl)-6-(4-Nitro-1H-Pyrazol-1-yl)Pyridine-3-Sulfonamide (1-157)

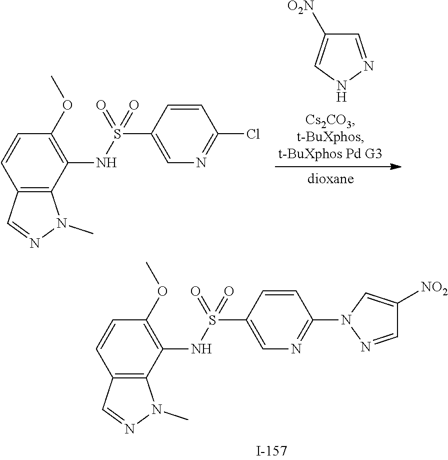 [SMILES: COc1ccc2cnn(C)c2c1NS(=O)(=O)c1ccc(Cl)nc1 ; O=[N+]([O-])c1cn[nH]c1 ; COc1ccc2cnn(C)c2c1NS(=O)(=O)c1ccc(-n2cc([N+](=O)[O-])cn2)nc1]

A mixture of 6-chloro-N-(6-methoxy-1-methylindazol-7-yl)pyridine-3-sulfonamide (40 mg, 0.113 mmol, 1 equiv.), Cs₂CO₃ (147.77 mg, 0.452 mmol, 4 equiv.), 4-nitropyrazole (15.38 mg, 0.136 mmol, 1.2 equiv.), t-BuXPhos (9.63 mg, 0.023 mmol, 0.2 equiv.) and t-BuXPhos 3rd generation precatalyst (18.04 mg, 0.023 mmol, 0.2 equiv.) in 1,4-dioxane (4 mL) was stirred for 5 h at 100° C. under a nitrogen atmosphere. The resulting mixture was filtered and the filter cake was washed with EtOAc (3 100 mL). The filtrate was concentrated under reduced pressure. The crude product (200 mg) was purified by prep-HPLC with the following conditions: column: XBridge Shield RP18 OBD, 30*150 mm, 5 m; mobile phase A: water (10 mmol/L NH₄HCO₃+0.05% NH3H₂O), mobile phase B: ACN; flow rate 60 mL/min; gradient: 28% B to 53% B in 7 min; wavelength: 254 nm/220 nm; Rt1(min): 6.77; number of runs: 3) to afford N-(6-methoxy-1-methylindazol-7-yl)-6-(4-nitropyrazol-1-yl)pyridine-3-sulfonamide (31.1 mg, 63.69%) as a white solid. LCMS: (ES, m/z): [M+H]⁺ 430; (400 MHz, DMSO-d₆) δ 3.18 (s, 3H), 4.27 (s, 3H), 6.83 (d, J 8.8 Hz, 1H), 7.68 (d, J 8.8 Hz, 1H), 8.00 (s, 1H), 8.21 (d, J 8.4 Hz, 1H), 8.31 (dd, J 8.4, 2.4 Hz, 1H), 8.72 (s, 1H), 8.74 (d, J2.0 Hz, 1H), 9.59 (s, 1H), 10.17 (s, 1H).

### Example 231—Synthesis of N-(1,2-Dimethyl-3-Oxo-2,3-Dihydro-1H-Indazol-7-yl)-6-(4-(Trifluoromethyl)-1H-Pyrazol-1-yl)Pyridine-3-Sulfonamide (I-158)

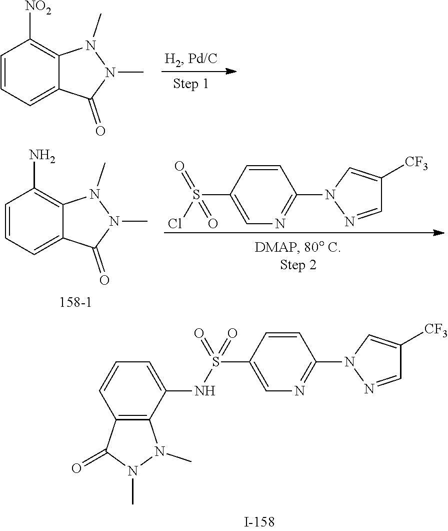

Step 1: Synthesis of 158-1. A mixture of 1,2-dimethyl-7-nitroindazol-3-one (150 mg, 0.724 mmol, 1 equiv.) and Pd/C (231.14 mg, 2.172 mmol, 3 equiv.) in EA (10 mL) was stirred for 1 h at room temperature under a hydrogen atmosphere. The resulting mixture was filtered. The filtrate was concentrated under reduced pressure. This resulted in 158-1 (120 mg, 93.54%) as a yellow solid.

Step 2: Synthesis of N-(1,2-dimethyl-3-oxo-2,3-dihydro-1H-indazol-7-yl)-6-(4-(trifluoromethyl)-1H-pyrazol-1-yl)pyridine-3-sulfonamide (1-158). A solution of 158-1 (395.72 mg, 1.269 mmol, 1.5 equiv.) and DMAP (20.68 mg, 0.169 mmol, 0.2 equiv.) in pyridine (2 mL) was stirred overnight at 50° C. under a nitrogen atmosphere. The reaction was quenched with water at room temperature. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. This resulted in N-(1,2-dimethyl-3-oxoindazol-7-yl)-6-[4-(trifluoromethyl)pyrazol-1-yl]pyridine-3-sulfonamide (6.3 mg, 1.63%) as a off-white solid. LCMS: (ES, m/z): [M+H]⁺ 453; ¹H NMR (400 MHz, DMSO-d₆): δ 3.29 (s, 3H), 3.34 (s, 3H), 6.95 (d, J7.2, 1.1 Hz, 1H), 7.07 (t, J 7.6 Hz, 1H), 7.59 (d, J7.6 Hz, 1H), 8.18 (d, J 8.4 Hz, 1H), 8.33 (dd, J8.8, 2.4 Hz, 1H), 8.42 (s, 1H), 8.72 (d, J2.0 Hz, 1H), 9.32 (s, 1H), 10.45 (s, 1H).

### Example 232—Synthesis of N-(6-Hydroxy-1-Methyl-1H-Indazol-7-yl)-6-(4-(Trifluoromethyl)-1H-Pyrazol-1-yl)Pyridine-3-Sulfonamide (I-159)

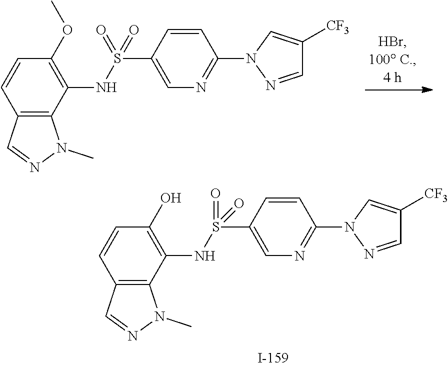 [SMILES: COc1ccc2cnn(C)c2c1NS(=O)(=O)c1ccc(-n2cc(C(F)(F)F)cn2)nc1 ; Cn1ncc2ccc(O)c(NS(=O)(=O)c3ccc(-n4cc(C(F)(F)F)cn4)nc3)c21]

A solution of N-(6-methoxy-1-methylindazol-7-yl)-6-[4-(trifluoromethyl)pyrazol-1-yl]pyridine-3-sulfonamide (200 mg, 0.442 mmol, 1 equiv.) in HBr in CH3COOH (40%) (5 mL) was stirred for 4 h at 100° C. under a nitrogen atmosphere. The resulting mixture was diluted with water (20 mL). The resulting mixture was extracted with EtOAc (3×20 mL). The combined organic layers were washed with brine (50 mL) and dried over anhydrous Na₂SO₄. After filtration, the filtrate was concentrated under reduced pressure. The crude product was purified by prep-HPLC with the following conditions: column: Xselect CSH C18 OBD, 19*250 mm 5 m; mobile phase A: water (0.1% FA), mobile phase B: ACN; flow rate 30 mL/min; gradient: 45% B to 57% B in 7 min; wavelength: 254 nm/220 nm; Rt1(min): 6.2) to afford N-(6-hydroxy-1-methylindazol-7-yl)-6-[4-(trifluoromethyl)pyrazol-1-yl]pyridine-3-sulfonamide (3.6 mg, 1.84%) as a white solid. LCMS: (ES, m/z): [M+H]⁺ 439; ¹H NMR (400 MHz, DMSO-d₆) δ 4.24 (s, 3H), 6.56 (d, J 8.4 Hz, 1H), 7.47 (d, J 8.4 Hz, 1H), 7.89 (s, 1H), 8.13 (dd, J 8.4, 0.8 Hz, 1H), 8.28 (dd, J 8.4, 2.4 Hz, 1H), 8.41 (s, 1H), 8.68 (dd, J 2.4, 0.8 Hz, 1H), 9.35 (t, J 1.2 Hz, 1H), 9.47 (s, 1H), 9.92 (s, 1H).

### Example 233—Synthesis of N-(6-Methoxy-1-Methyl-1H-Indazol-7-yl)-6-(3-Methyl-2-Oxo-2,3-Dihydro-1H-Imidazol-1-yl)Pyridine-3-Sulfonamide (I-160)

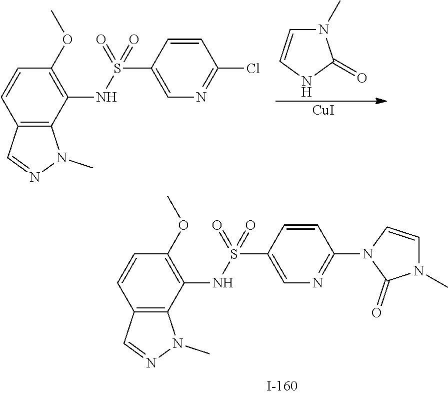

A mixture of 6-chloro-N-(6-methoxy-1-methylindazol-7-yl)pyridine-3-sulfonamide (170 mg, 0.482 mmol, 1 equiv.), 1-methyl-3H-imidazol-2-one (142 mg, 1.447 mmol, 3.00 equiv.), Pd₂(dba)₄ (110 mg, 0.096 mmol, 0.20 equiv.), XantPhos (55 mg, 0.095 mmol, 0.20 equiv.), KI (80 mg, 0.482 mmol, 1.00 equiv.) and Cs₂CO₃ (470 mg, 1.443 mmol, 2.99 equiv.) in DMF (5 mL) was stirred overnight at 90° C. under a nitrogen atmosphere. The resulting mixture was filtered and the filter cake was washed with MeCN (3×3 mL). The filtrate was concentrated under reduced pressure. The residue was purified by reversed-phase flash chromatography with the following conditions: column: C18 silica gel; mobile phase: MeCN in water, 10% to 50% gradient in 10 min; detector: UV 254 nm. This resulted in N-(6-methoxy-1-methyl-1H-indazol-7-yl)-6-(3-methyl-2-oxo-2,3-dihydro-1H-imidazol-1-yl)pyridine-3-sulfonamide (5.1 mg, 2.50%) as an off-white solid. LCMS: (ES, m/z): [M+H]⁺ 415; ¹H NMR (400 MHz, DMSO-d₆) δ 3.16 (s, 3H), 3.23 (s, 3H), 4.26 (s, 3H), 6.77-6.86 (m, 2H), 7.37 (d, J3.2 Hz, 1H), 7.63 (d, J8.8 Hz, 1H), 7.98 (s, 1H), 8.13 (dd, J8.8, 2.4 Hz, 1H), 8.50-8.58 (m, 2H), 9.54 (s, 1H).

### Example 234—Synthesis of N-(6-Methoxy-1-Methyl-1H-Indazol-7-yl)-6-(5-Methyl-1,2,4-Oxadiazol-3-yl)Pyridine-3-Sulfonamide (I-161)

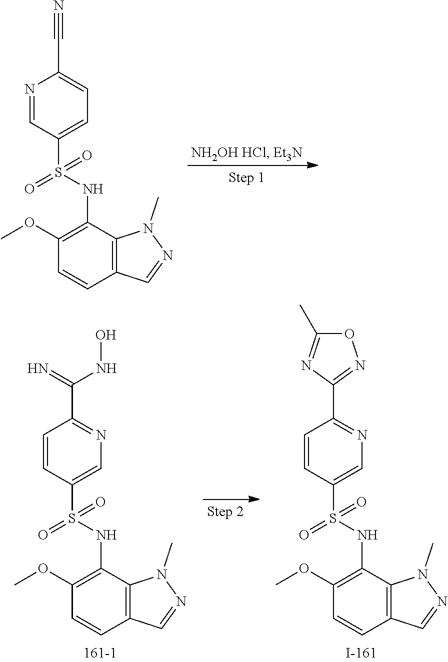 [SMILES: COc1ccc2cnn(C)c2c1NS(=O)(=O)c1ccc(C#N)nc1 ; COc1ccc2cnn(C)c2c1NS(=O)(=O)c1ccc(C(=N)NO)nc1 ; COc1ccc2cnn(C)c2c1NS(=O)(=O)c1ccc(-c2noc(C)n2)nc1]

Step 1: Synthesis of 161-1. To a stirred solution of 6-cyano-N-(6-methoxy-1-methylindazol-7-yl)pyridine-3-sulfonamide (120 mg, 0.349 mmol, 1 equiv.) and NH₂OH·HCl (73 mg, 1.047 mmol, 3 equiv.) in MeOH (3 mL) was added TEA (106 mg, 1.047 mmol, 3 equiv.). The resulting mixture was stirred for 4 h at 60° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure to afford N-hydroxy-5-[(6-methoxy-1-methylindazol-7-yl)sulfamoyl]pyridine-2-carboximidamide (130 mg, 98.83%) as an off-white solid.

Step 2: Synthesis of N-(6-methoxy-1-methyl-1H-indazol-7-yl)-6-(5-methyl-1,2,4-oxadiazol-3-yl)pyridine-3-sulfonamide (I-161). A solution of N-hydroxy-5-[(6-methoxy-1-methylindazol-7-yl)sulfamoyl]pyridine-2-carboximidamide (120 mg, 0.319 mmol, 1 equiv.) and acetic anhydride (162 mg, 1.595 mmol, 5 equiv.) in AcOH (2 mL) was stirred for 1 h at 80° C. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions (flow rate 60 mL/min; gradient: 10% B to 37% B in 7 min; wavelength: 254 nm/220 nm; Rt1(min): 7.0; number of runs: 4) to afford N-(6-methoxy-1-methylindazol-7-yl)-6-(5-methyl-1,2,4-oxadiazol-3-yl)pyridine-3-sulfonamide (54.8 mg, 40.91%) as a white solid. LCMS: (ES, m/z): [M+H]⁺ 401; ¹H NMR (400 MHz, DMSO-d₆) δ 2.72 (s, 3H), 3.11 (s, 3H), 4.27 (s, 3H), 6.82 (d, J 8.8 Hz, 1H), 7.68 (d, J 8.8 Hz, 1H), 8.00 (s, 1H), 8.22(dd, J 8.4 Hz, 2.0 Hz, 1H), 8.27 (d, J 8.0 Hz, 1H), 8.94 (d, J 2.4 Hz, 1H), 10.18 (s, 1H).

### Example 235—Synthesis of N-(6-Methoxy-1-Methyl-1H-Indazol-7-yl)-N-Methyl-6-(4-(Trifluoromethyl)-1H-Pyrazol-1-yl)Pyridine-3-Sulfonamide (I-162)

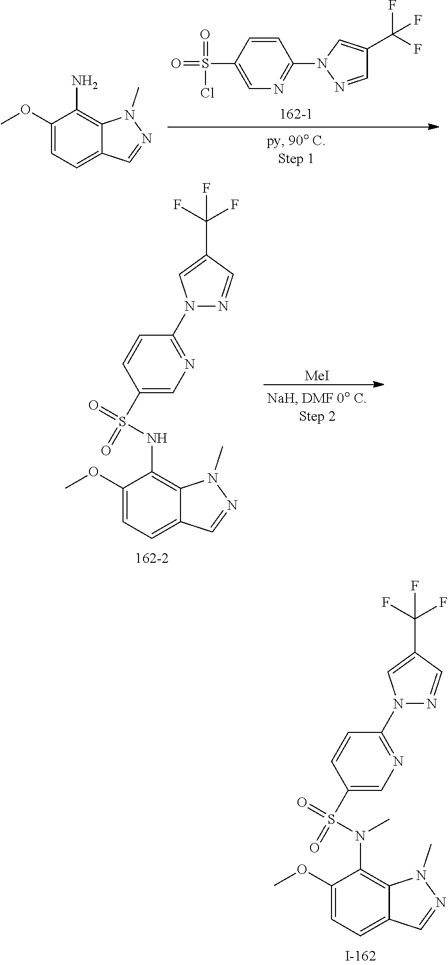

Step 1: Synthesis of 162-2. A solution of 6-[4-(trifluoromethyl)pyrazol-1-yl]pyridine-3-sulfonyl chloride (527.62 mg, 1.694 mmol, 1.50 equiv.) and 6-methoxy-1-methylindazol-7-amine (200 mg, 1.129 mmol, 1.00 equiv.) in pyridine (10 mL) was stirred for 2 h at 90° C. under a nitrogen atmosphere. The resulting mixture was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm to afford N-(6-methoxy-1-methylindazol-7-yl)-6-[4-(trifluoromethyl)pyrazol-1-yl]pyridine-3-sulfonamide (220 mg, 43.09%) as an off-white solid.

Step 2: Synthesis of N-(6-methoxy-1-methyl-1H-indazol-7-yl)-N-methyl-6-(4-(trifluoromethyl)-1H-pyrazol-1-yl)pyridine-3-sulfonamide (I-162). To a stirred solution of N-(6-methoxy-1-methylindazol-7-yl)-6-[4-(trifluoromethyl)pyrazol-1-yl]pyridine-3-sulfonamide (100 mg, 0.221 mmol, 1 equiv.) and NaH (13.26 mg, 0.552 mmol, 2.5 equiv.) in DMF (2 mL) was stirred for 30 min at 0° C. under a nitrogen atmosphere. Then Mel (47.06 mg, 0.332 mmol, 1.5 equiv.) was added. The resulting mixture was stirred for 2 h at 0° C. under a nitrogen atmosphere. The reaction was quenched with water/ice at 0° C. The resulting mixture was extracted with EtOAc (3×40 mL). The combined organic layers were washed with brine (3×40 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm. The crude product (110 mg) was purified by prep-HPLC with the following conditions: column: XBridge Prep OBD C18, 30*150 mm, 5 m; mobile phase A: water (10 mmol/L NH₄HCO₃)+0.05% NH3. H₂O, mobile phase B: ACN; flow rate 60 mL/min; gradient: 45% B to 72% B in 7 min; wavelength: 254 nm/220 nm; Rt1(min): 7.37; number of runs: 2) to afford N-(6-methoxy-1-methylindazol-7-yl)-N-methyl-6-[4-(trifluoromethyl)pyrazol-1-yl]pyridine-3-sulfonamide (63 mg, 60.80%) as a white solid. LCMS: (ES, m/z): [M+H]⁺ 467; ¹H NMR (400 MHz, DMSO-d₆) δ 3.26 (s, 3H), 3.34 (s, 3H), 4.24 (s, 3H), 6.91 (d, J8.8 Hz, 1H), 7.76 (d, J 8.8 Hz, 1H), 8.02 (s, 1H), 8.21 (d, J 8.4 Hz, 1H), 8.35 (dd, J 8.4, 2.4 Hz, 1H), 8.43 (s, 1H), 8.76 (d, J2.0 Hz, 1H), 9.33 (s, 1H).

### Example 236—Synthesis of N-(2-(Dimethylamino)Ethyl)-N-(6-Methoxy-1-Methyl-1H-Indazol-7-yl)-6-(4-(Trifluoromethyl)-1H-Pyrazol-1-yl)Pyridine-3-Sulfonamide (I-163)

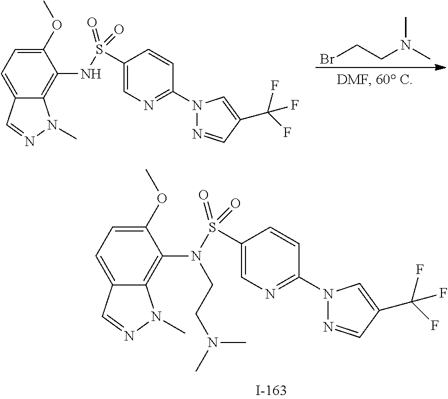

To a stirred solution of N-(6-methoxy-1-methylindazol-7-yl)-6-[4-(trifluoromethyl)pyrazol-1-yl]pyridine-3-sulfonamide (90 mg, 0.199 mmol, 1 equiv.) and (2-bromoethyl)dimethylamine hydrobromide (69.51 mg, 0.298 mmol, 1.5 equiv.) in DMF (2 mL) was added Cs₂CO₃ (194.45 mg, 0.597 mmol, 3 equiv.) in portions. The resulting mixture was stirred for 2 h at 60° C. The reaction was quenched with water at room temperature. The resulting mixture was extracted with EtOAc (3×40 mL). The combined organic layers were washed with brine (3×40 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm. The crude product (120 mg) was purified by prep-HPLC with the following conditions: column: XBridge Prep OBD C18, 30*150 mm, 5 m; mobile phase A: water (10 mmol/L NH₄HCO₃+0.05% NH3H₂O), mobile phase B: ACN; flow rate 60 mL/min; gradient: 46% B to 71% B in 7 min; wavelength: 220 nm; Rt1(min): 7.32; number of runs: 2) to afford N-[2-(dimethylamino)ethyl]-N-(6-methoxy-1-methylindazol-7-yl)-6-[4-(trifluoromethyl)pyrazol-1-yl]pyridine-3-sulfonamide (54.7 mg, 52.42%) as a white solid. LCMS: (ES, m/z): [M+H]⁺ 524; ¹H NMR (400 MHz, DMSO-d₆) δ 2.00 (s, 6H), 2.26 (t, J 6.0 Hz, 2H), 3.22 (s, 3H), 3.45-3.49 (m, 1H), 4.05-4.10 (m, 1H), 4.32 (s, 3H), 6.88 (d, J8.8 Hz, 1H), 7.76 (d, J 8.8 Hz, 1H), 8.01 (s, 1H), 8.18 (d, J8.8 Hz, 1H), 8.33 (dd, J 8.8, 2.4 Hz, 1H), 8.42 (s, 1H), 8.71 (d, J2.0 Hz, 1H), 9.33 (s, 1H).

### Example 237—Synthesis of N-(3-(Dimethylamino)Propyl)-N-(6-Methoxy-1-Methyl-1H-Indazol-7-yl)-6-(4-(Trifluoromethyl)-1H-Pyrazol-1-yl)Pyridine-3-Sulfonamide (I-164)

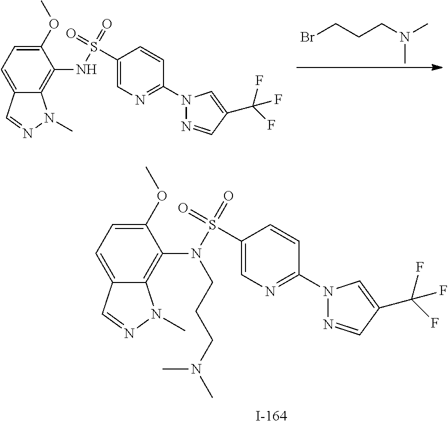

To a stirred solution of N-(6-methoxy-1-methylindazol-7-yl)-6-[4-(trifluoromethyl)pyrazol-1-yl]pyridine-3-sulfonamide (90 mg, 0.199 mmol, 1 equiv.) and (3-bromopropyl)dimethylamine (73 mg, 0.298 mmol, 1.5 equiv.) in DMF (2 mL) was added Cs₂CO₃ (194.45 mg, 0.597 mmol, 3 equiv.). The resulting mixture was stirred for 2 h at 60° C. under a nitrogen atmosphere. The resulting mixture was diluted with water (10 mL) and extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions (flow rate 60 mL/min; gradient: 40% B to 68% B in 7 min; wavelength: 254 nm/220 nm; Rt1(min): 7.57) to afford N-[3-(dimethylamino)propyl]-N-(6-methoxy-1-methylindazol-7-yl)-6-[4-(trifluoromethyl)pyrazol-1-yl]pyridine-3-sulfonamide (70.6 mg, 65.95%) as a white solid. LCMS: (ES, m/z): [M+H]⁺ 538.1; H NMR (400 MHz, DMSO-d₆) δ 1.25-1.56 (m, 1H), 1.58-1.87 (m, 1H), 1.99 (s, 6H), 2.14 (t, J 6.8 Hz, 2H), 3.25 (s, 3H), 3.41-3.51 (m, 1H), 3.85-4.06 (m, 1H), 4.27 (s, 3H), 6.92 (d, J 8.8 Hz, 1H), 7.78 (d, J 8.8 Hz, 1H), 8.05 (s, 1H), 8.20 (d, J 8.8 Hz, 1H), 8.32 (dd, J 8.8, 2.4 Hz, 1H), 8.42 (s, 1H), 8.70 (d, J 2.0 Hz, 1H), 9.33 (s, 1H).

### Example 238—Synthesis of N-(6-Methoxy-1-Methyl-1H-Indazol-7-yl)-N-Methyl-6-(1-Methyl-5-(Trifluoromethyl)-1H-Pyrazol-3-yl)Pyridine-3-Sulfonamide (I-165)

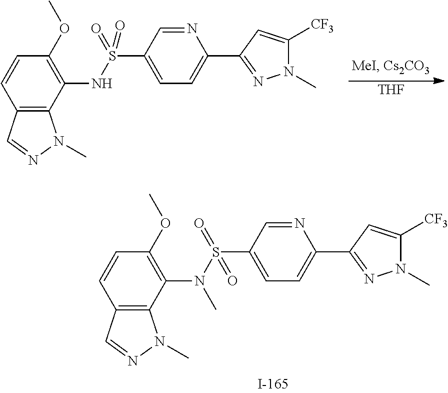 [SMILES: COc1ccc2cnn(C)c2c1NS(=O)(=O)c1ccc(-c2cc(C(F)(F)F)n(C)n2)nc1 ; COc1ccc2cnn(C)c2c1N(C)S(=O)(=O)c1ccc(-c2cc(C(F)(F)F)n(C)n2)nc1 ; "THF"]

To a stirred solution of N-(6-methoxy-1-methylindazol-7-yl)-6-[2-methyl-5-(trifluoromethyl)pyrazol-3-yl] pyridine-3-sulfonamide (15 mg, 0.032 mmol, 1 equiv.) and Cs₂CO₃ (31.43 mg, 0.096 mmol, 3 equiv.) in THF (2 mL) was added methyl iodide (9.13 mg, 0.064 mmol, 2 equiv.) dropwise at room temperature under a nitrogen atmosphere. The resulting mixture was stirred for 2 h at room temperature under a nitrogen atmosphere. The resulting mixture was diluted with water (10 mL). The resulting mixture was extracted with EtOAc (3×5 mL). The combined organic layers were washed with brine (10 mL) and dried over anhydrous Na₂SO₄. After filtration, the filtrate was concentrated under reduced pressure. The crude product (30 mg) was purified by prep-HPLC with the following conditions: column: XBridge Prep Phenyl OBD, 19*250 mm, 5 m; mobile phase A: water(10 mmol/L NH₄HCO₃+0.1% NH₃·H₂O), mobile phase B: ACN; flow rate 25 mL/min; gradient: 37% B to 62% B in 11 min; wavelength: 220/254 nm; Rt1(min): 10.45; injection volume: 0.4 mL; number of runs: 4) to afford N-(6-methoxy-1-methylindazol-7-yl)-N-methyl-6-[2-methyl-5-(trifluoromethyl)pyrazol-3-yl]pyridine-3-sulfonamide (6.7 mg, 43.10%) as a white solid. LCMS: (ES, m/z): [M+H]⁺ 481; ¹H NMR (400 MHz, DMSO-d₆) δ 3.18 (s, 3H), 3.33 (s, 3H), 4.11 (s, 3H), 4.24 (s, 3H), 6.89 (d, J 8.8 Hz, 1H), 7.53 (s, 1H), 7.76 (d, J 8.8 Hz, 1H), 8.02 (s, 1H), 8.16 (d, J 8.8 Hz, 1H), 8.18 (dd, J 8.4 Hz, 2.0 Hz, 1H), 8.82 (dd, J 2.4, 1.2 Hz, 1H).

### Example 239—Synthesis of N-(6-Methoxy-1-Methyl-1H-Indazol-7-yl)-6-(3-Methyl-1H-1,2,4-Triazol-5-yl)Pyridine-3-Sulfonamide (I-166)

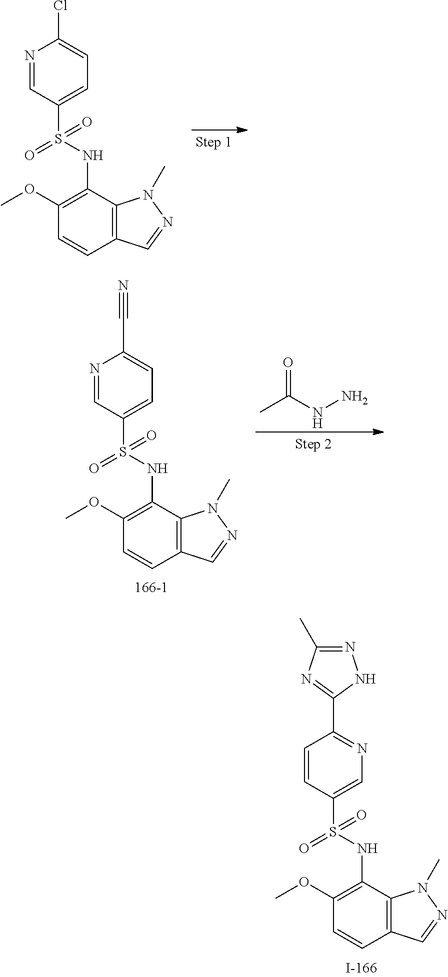

Step 1: Synthesis of 166-1. To a stirred solution of 6-chloro-N-(6-methoxy-1-methylindazol-7-yl)pyridine-3-sulfonamide (400 mg, 1.134 mmol, 1 equiv.) and Zn(CN)₂ (199 mg, 1.701 mmol, 1.5 equiv.) in NMP (2 mL) were added Pd₂(dba)₃ (103 mg, 0.113 mmol, 0.1 equiv.) and dppf (125 mg, 0.227 mmol, 0.2 equiv.). The resulting mixture was stirred for 3 h at 140° C. under a nitrogen atmosphere. The resulting mixture was diluted with water (10 mL) and extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm to afford 6-cyano-N-(6-methoxy-1-methylindazol-7-yl)pyridine-3-sulfonamide (350 mg, 89.90%) as an orange solid.

Step 2: Synthesis of N-(6-methoxy-1-methyl-1H-indazol-7-yl)-6-(3-methyl-11H-1,2,4-triazol-5-yl)pyridine-3-sulfonamide (1-166). A solution of methyl 5-[(6-methoxy-1-methylindazol-7-yl)sulfamoyl]pyridine-2-carboximidate (120 mg, 0.320 mmol, 1 equiv.) in MeOH (2 mL) was treated with acetohydrazide (71 mg, 0.960 mmol, 3 equiv.) and TsOH (55 mg, 0.320 mmol, 1 equiv.). The resulting mixture was stirred for 16 h at 70° C. The resulting mixture was diluted with water (10 mL) and extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: Xselect CSH C18 OBD, 30*150 mm 11 m; mobile phase A: water (0.1% FA), mobile phase B: ACN; flow rate 60 mL/min; gradient: 15% B to 25% B in 10 min; wavelength: 254 nm/220 nm; Rt1(min): 9.43) to afford N-(6-methoxy-1-methylindazol-7-yl)-6-(5-methyl-2H-1,2,4-triazol-3-yl)pyridine-3-sulfonamide (35.6 mg, 26.93%) as a white solid. LCMS: (ES, m/z): [M+H]⁺ 400.1; ¹H NMR (400 MHz, DMSO-d₆) δ 2.42 (s, 3H), 3.11 (s, 3H), 4.27 (s, 3H), 6.81 (d, J 8.8 Hz, 1H), 7.67 (d, J 8.8 Hz, 1H), 7.99 (s, 1H), 8.12 (s, 1H), 8.21 (d, J 8.4 Hz, 1H), 8.80 (d, J 2.4 Hz, 1H), 10.06 (s, 1H), 14.05 (s, 1H).

### Example 240—Synthesis of N-(6-Methoxy-1-Methyl-1H-Indazol-7-yl)-6-(3-(Trifluoromethyl)-1H-1,2,4-Triazol-5-yl)Pyridine-3-Sulfonamide (I-167)

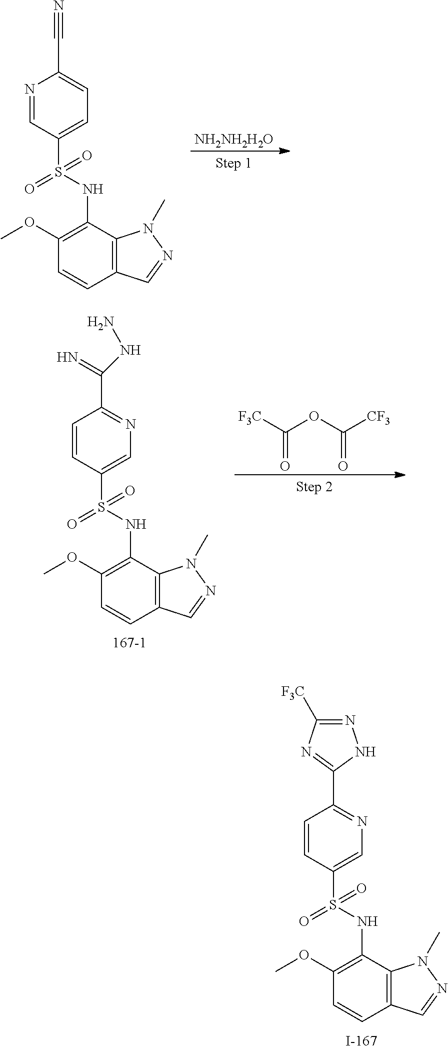

Step 1: Synthesis of 167-1. To a stirred solution of 6-cyano-N-(6-methoxy-1-methylindazol-7-yl)pyridine-3-sulfonamide (150 mg, 0.437 mmol, 1 equiv.) and NH₂NH₂. H₂O (65 mg, 1.311 mmol, 3 equiv.) in MeOH (3 mL) was added TEA (132 mg, 1.311 mmol, 3 equiv.). The resulting mixture was stirred for 16 h at 80° C. The resulting mixture was concentrated under reduced pressure to afford N-amino-5-[(6-methoxy-1-methylindazol-7-yl)sulfamoyl]pyridine-2-carboximidamide (150 mg, 91.46%) as an off-white solid.

Step 2: Synthesis of N-(6-methoxy-1-methyl-1H-indazol-7-yl)-6-(3-(trifluoromethyl)-1H-1,2,4-triazol-5-yl)pyridine-3-sulfonamide (1-167). A solution of N-amino-5-[(6-methoxy-1-methylindazol-7-yl)sulfamoyl]pyridine-2-carboximidamide (150 mg, 0.400 mmol, 1 equiv.) and trifluoroacetic anhydride (251.76 mg, 1.200 mmol, 3 equiv.) in TFA (2 mL) was stirred for 2 h at 80° C. The resulting mixture was diluted with water (10 mL) and extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: Xselect CSH C18 OBD, 30*150 mm 5 m; mobile phase A: water (0.1% FA), mobile phase B: ACN; flow rate 60 mL/min; gradient: 40% B to 50% B in 7 min; wavelength: 254 nm/220 nm; Rt1(min): 5.9) to afford N-(6-methoxy-1-methylindazol-7-yl)-6-[5-(trifluoromethyl)-2H-1,2,4-triazol-3-yl]pyridine-3-sulfonamide (73.3 mg, 40.42%) as a white solid. LCMS: (ES, m/z): [M+H]⁺ 454; ¹H NMR (400 MHz, DMSO-d₆) δ 3.13 (s, 3H), 4.28 (s, 3H), 6.82 (d, J 8.8 Hz, 1H), 7.69 (d, J 8.8 Hz, 1H), 8.00 (s, 1H), 8.26 (dd, J 8.0, 2.4 Hz, 1H), 8.35 (d, J 8.4 Hz, 1H), 8.89 (d, J 2.4 Hz, 1H), 10.20 (s, 1H), 16.00 (s, 1H).

EXAMPLE 241—SYNTHESIS OF 6-(4,5-DIMETHYL-4H-1,2,4-TRIAZOL-3-YL)-N-(6-METHOXY-1-METHYL-1H-INDAZOL-7-YL)PYRIDINE-3-SULFONAMIDE (I-168)

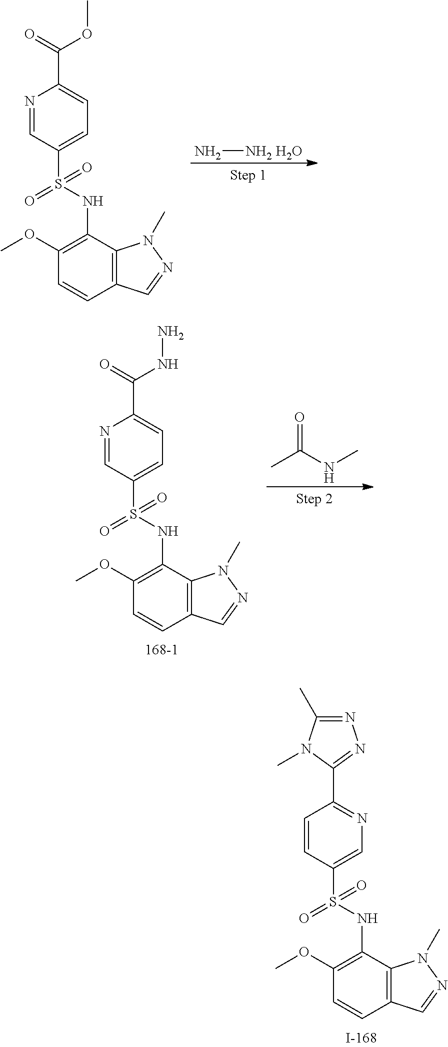

Step 1: Synthesis of 168-1. A mixture of methyl 5-[(6-methoxy-1-methylindazol-7-yl) sulfamoyl] pyridine-2-carboxylate (500 mg, 1.32 mmol, 1.00 equiv.) and hydrazine hydrate (665 mg, 13.2 mmol, 10.0 equiv.) in EtOH (10 mL) was stirred for 2 h at room temperature under a nitrogen atmosphere. The resulting mixture was concentrated under reduced pressure carefully. The residue was purified by silica gel column chromatography, eluted with PE/EA (1:1) to afford 6-(hydrazinecarbonyl)-N-(6-methoxy-1-methylindazol-7-yl) pyridine-3-sulfonamide (300 mg, 60%) as a yellow solid.

Step 2: Synthesis of 6-(4,5-dimethyl-4H-1,2,4-triazol-3-yl)-N-(6-methoxy-1-methyl-1H-indazol-7-yl)pyridine-3-sulfonamide (1-168). To a stirred mixture of N-methylacetamide (29 mg, 0.399 mmol, 3.00 equiv.) and 2,6-lutidine (28 mg, 0.266 mmol, 2.00 equiv.) in DCM (3 mL) was added oxalyl chloride (50 mg, 0.399 mmol, 3.00 equiv.) dropwise at 0° C. under a nitrogen atmosphere. The resulting mixture was stirred for 1 h at 0° C. under a nitrogen atmosphere. To the above mixture was added 6-(hydrazinecarbonyl)-N-(6-methoxy-1-methylindazol-7-yl)pyridine-3-sulfonamide (50 mg, 0.133 mmol, 1 equiv.) dropwise at 0° C. The resulting mixture was stirred for an additional 3 h at room temperature. The resulting mixture was concentrated under reduced pressure. The crude product was purified by prep-HPLC with the following conditions: column: XBridge Prep OBD C18, 30*150 mm, 5 m; mobile phase A: water (10 mmol/L NH₄HCO₃)+0.05% NH₃. H₂O, mobile phase B: ACN; flow rate 60 mL/min; gradient: 6% B to 30% B in 7 min; wavelength: 254 nm/220 nm) to afford 6-(4,5-dimethyl-1,2,4-triazol-3-yl)-N-(6-methoxy-1-methylindazol-7-yl) pyridine-3-sulfonamide (18.2 mg, 33%) as a white solid. LCMS (ESI, m/z): [M+H]⁺414; ¹H NMR (400 MHz, chloroform-d) δ 2.73 (s, 3H), 3.23 (s, 3H), 4.10 (s, 3H), 4.43 (s, 3H), 6.54 (s, 1H), 6.59 (d, J 8.8 Hz, 1H), 7.63 (d, J8.8 Hz, 1H), 7.95 (s, 1H), 8.02 (dd, J8.4, 2.4 Hz, 1H), 8.35 (d, J8.4 Hz, 1H), 8.84 (d, J2.0 Hz, 1H).

### Example 242—Synthesis of N-(6-Methoxy-1-Methyl-1H-Indazol-7-yl)-6-(3-Methyl-1,2,4-Oxadiazol-5-yl)Pyridine-3-Sulfonamide (I-169)

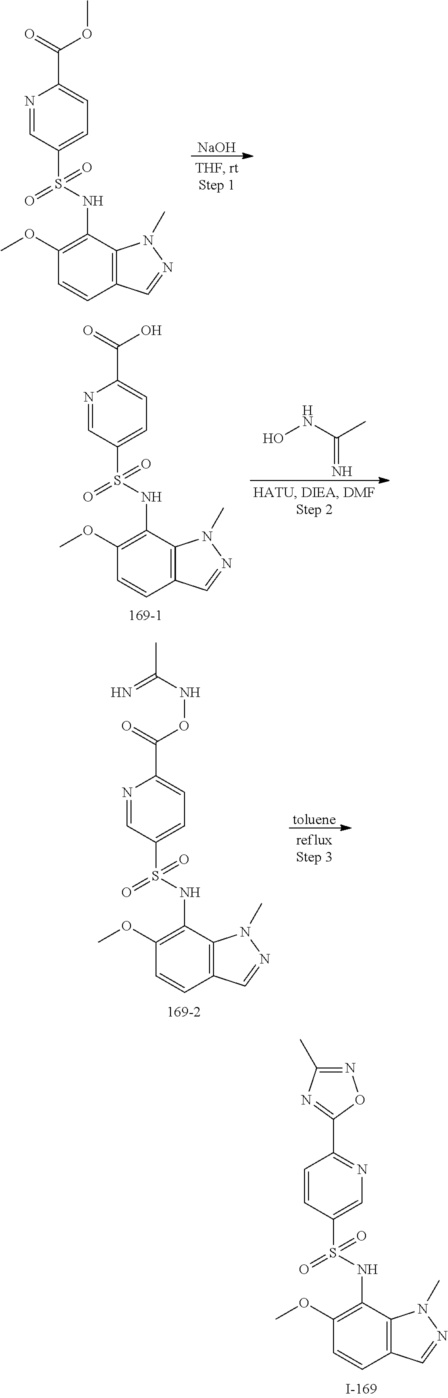

Step 1: Synthesis of 169-1. A solution of methyl 5-[(6-methoxy-1-methylindazol-7-yl)sulfamoyl]pyridine-2-carboxylate (200 mg, 0.531 mmol, 1 equiv.) and NaOH (63.76 mg, 1.593 mmol, 3 equiv.) in THE (3 mL) and H₂O (1 mL) was stirred for 2 h at room temperature. The mixture was neutralized to pH 7 with conc. HCl. The precipitated solids were collected by filtration and washed with water (5×5 mL). The resulting solid was dried under vacuum. This resulted in 5-[(6-methoxy-1-methylindazol-7-yl)sulfamoyl]pyridine-2-carboxylic acid (170 mg, 78.49%) as a yellow solid.

Step 2: Synthesis of 169-2. A solution of 5-[(6-methoxy-1-methylindazol-7-yl)sulfamoyl]pyridine-2-carboxylic acid (170 mg, 0.469 mmol, 1 equiv.) in DMF (3 mL) was treated with DIEA (181.91 mg, 1.407 mmol, 3 equiv.) and HATU (267.58 mg, 0.704 mmol, 1.5 equiv.) for 30 min at room temperature followed by the addition of N-hydroxyethanimidamide (52.13 mg, 0.704 mmol, 1.5 equiv.). The resulting mixture was stirred for 2 h at room temperature. The reaction was quenched with water at room temperature. The resulting mixture was extracted with EtOAc (4×50 mL). The combined organic layers were washed with brine (4×40 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm. This resulted in ethanimidamido 5-[(6-methoxy-1-methylindazol-7-yl)sulfamoyl]pyridine-2-carboxylate (130 mg, 65.43%) as a light yellow solid.

Step 3: Synthesis of N-(6-methoxy-1-methyl-1H-indazol-7-yl)-6-(3-methyl-1,2,4-oxadiazol-5-yl)pyridine-3-sulfonamide (I-169). A solution of ethanimidamido 5-[(6-methoxy-1-methylindazol-7-yl)sulfamoyl]pyridine-2-carboxylate (120 mg, 0.287 mmol, 1 equiv.) in toluene (4 mL) was stirred for 2 h at 110° C. The resulting mixture was concentrated under vacuum. The residue was purified by reversed-phase flash chromatography with the following conditions: column: C18 silica gel; mobile phase: MeCN in water (0.1% TFA), 10% to 50% gradient in 10 min; detector: UV 254 nm. The crude product (80 mg) was purified by prep-HPLC with the following conditions: column: XBridge Shield RP18 OBD, 30*150 mm, 5 m; mobile phase A: water (10 mmol/L NH₄HCO₃+0.05% NH3H₂O), mobile phase B: ACN; flow rate 60 mL/min; gradient: 10% B to 35% B in 7 min; wavelength: 254 nm/220 nm; Rt1(min): 7; number of runs: 3) to afford N-(6-methoxy-1-methylindazol-7-yl)-6-(3-methyl-1,2,4-oxadiazol-5-yl)pyridine-3-sulfonamide (22.6 mg, 19.33%) as a light yellow solid. LCMS: (ES, m/z): [M+H]⁺ 401; (400 MHz, DMSO-d₆) δ 2.51 (s, 3H), 3.12 (s, 3H), 4.27 (s, 3H), 6.82 (d, J8.8 Hz, 1H), 7.68 (d, J8.8 Hz, 1H), 8.00 (s, 1H), 8.28 (dd, J8.4, 2.4 Hz, 1H), 8.42 (d, J8.4 Hz, 1H), 8.99 (dd, J2.4 Hz, 1H), 10.28 (s, 1H).

### Example 243—Synthesis of N-(6-Methoxy-1-Methyl-1H-Indazol-7-yl)-6-(5-Methyl-1,3,4-Oxadiazol-2-yl)Pyridine-3-Sulfonamide (I-170)

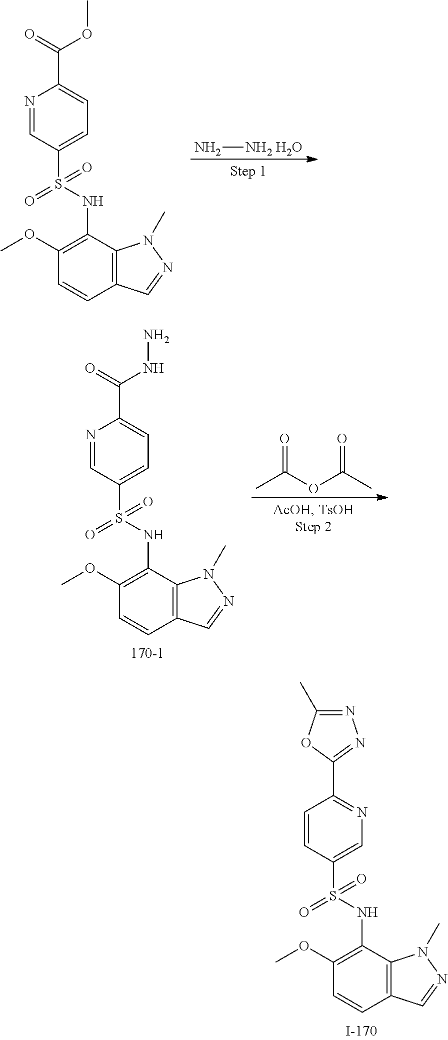

Step 1: Synthesis of 170-1. A mixture of methyl 5-[(6-methoxy-1-methylindazol-7-yl) sulfamoyl] pyridine-2-carboxylate (500 mg, 1.32 mmol, 1.00 equiv.) and hydrazine hydrate (665 mg, 13.2 mmol, 10.0 equiv.) in EtOH (10 mL) was stirred for 2 h at room temperature under a nitrogen atmosphere. The resulting mixture was concentrated under reduced pressure. The residue was purified by silica gel column chromatography, eluted with PE/EA (1:1) to afford 6-(hydrazinecarbonyl)-N-(6-methoxy-1-methylindazol-7-yl) pyridine-3-sulfonamide (300 mg, 60%) as a yellow solid.

Step 2: Synthesis of N-(6-methoxy-1-methyl-1H-indazol-7-yl)-6-(5-methyl-1,3,4-oxadiazol-2-yl)pyridine-3-sulfonamide (1-170). A mixture of 6-(hydrazinecarbonyl)-N-(6-methoxy-1-methylindazol-7-yl) pyridine-3-sulfonamide (100 mg, 0.266 mmol, 1.00 equiv.), acetic anhydride (81 mg, 0.798 mmol, 3.00 equiv.) and TsOH (137 mg, 0.798 mmol, 3.00 equiv.) in AcOH (2 mL) was stirred overnight at 140° C. under a nitrogen atmosphere. The resulting mixture was concentrated under reduced pressure. The crude product was purified by prep-HPLC with the following conditions: column: XBridge Shield RP18 OBD, 30*150 mm, 5 m; mobile phase A: water (10 mmol/L NH₄HCO₃+0.05% NH₃H₂O), mobile phase B: ACN; flow rate 60 mL/min; gradient: 10% B to 25% B in 7 min; wavelength: 254 nm/220 nm, to afford N-(6-methoxy-1-methylindazol-7-yl)-6-(5-methyl-1,3,4-oxadiazol-2-yl) pyridine-3-sulfonamide (3.4 mg, 3%) as a white solid. LCMS (ESI, m/z): [M+H]⁺ 401; ¹H NMR (400 MHz, Chloroform-d) δ 2.70 (s, 3H), 3.23 (s, 3H), 4.43 (s, 3H), 6.51 (s, 1H), 6.58 (d, J8.8 Hz, 1H), 7.63 (d, J8.8 Hz, 1H), 7.95 (s, 1H), 8.00 (dd, J8.4, 2.4 Hz, 1H), 8.27 (d, J8.4 Hz, 1H), 8.94 (d, J 1.6 Hz, 1H).

### Example 244—Synthesis of 6-(1,5-Dimethyl-1,2,4-Triazol-3-yl)-N-(6-Methoxy-1-Methylindazol-7-yl)Pyridine-3-Sulfonamide (I-176)

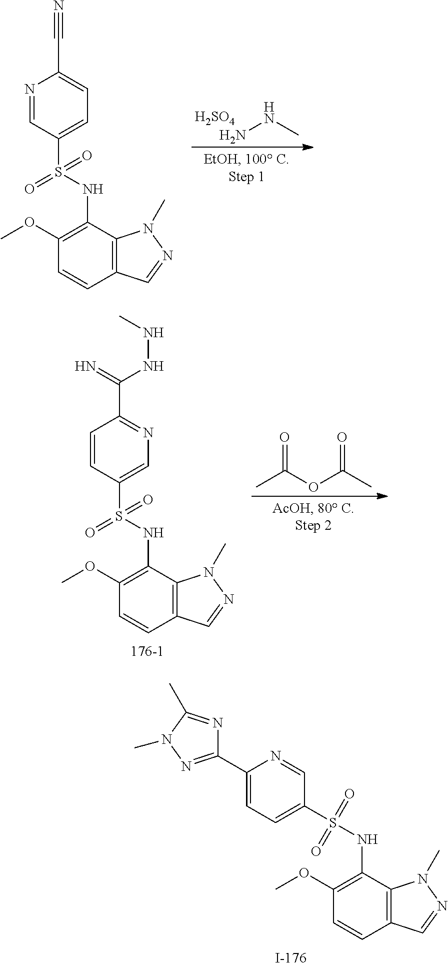

Step 1: Synthesis of 176-1. A solution of 6-cyano-N-(6-methoxy-1-methylindazol-7-yl)pyridine-3-sulfonamide (300 mg, 0.874 mmol, 1 equiv.), methylhydrazine and sulfuric acid (629.73 mg, 4.370 mmol, 5 equiv.) in EtOH (6 mL) was stirred for 16 h at 100° C. under a nitrogen atmosphere. The reaction was quenched with water at room temperature. The resulting mixture was extracted with EtOAc (2×50 mL). The combined organic layers were washed with brine (2×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm to afford 5-[(6-methoxy-1-methylindazol-7-yl)sulfamoyl]-N-(methylamino)pyridine-2-carboximidamide (80 mg, 23.51%) as a light yellow solid.

Step 2: Synthesis of 6-(1,5-dimethyl-1,2,4-triazol-3-yl)-N-(6-methoxy-1-methylindazol-7-yl)pyridine-3-sulfonamide (1-176). A solution of 5-[(6-methoxy-1-methylindazol-7-yl)sulfamoyl]-N-(methylamino)pyridine-2-carboximidamide (40 mg, 0.103 mmol, 1 equiv.) and acetic anhydride (31.46 mg, 0.309 mmol, 3 equiv.) in AcOH (2 mL) was stirred for 2 h at 80° C. The reaction was quenched with water at room temperature. The resulting mixture was extracted with EtOAc (3×20 mL). The combined organic layers were washed with brine (3×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 10% to 50% gradient in 10 min; detector: UV 254 nm. The crude product (30 mg) was purified by prep-HPLC with the following conditions: column: XBridge Shield RP18 OBD, 30*150 mm, 5 m; mobile phase A: water(10 mmol/L NH₄HCO₃+0.05% NH3H₂O), mobile phase B: ACN; flow rate 60 mL/min; gradient: 7% B to 30% B in 7 min; wavelength: 254 nm/220 nm; Rt1(min): 6.82; number of runs: 2) to afford 6-(1,5-dimethyl-1,2,4-triazol-3-yl)-N-(6-methoxy-1-methylindazol-7-yl)pyridine-3-sulfonamide (8 mg, 18.78%) as a white solid. LCMS: (ES, m/z): [M+H]⁺ 414; ¹H NMR (400 MHz, DMSO-d₆)δ 2.48 (s, 3H), 3.09 (s, 3H), 3.88 (s, 3H), 4.27 (s, 3H), 6.81 (d, J8.8 Hz, 1H), 7.67 (d, J8.8 Hz, 1H), 7.99 (s, 1H), 8.07 (dd, J8.4, 2.4 Hz, 1H), 8.18 (dd, J8.4 Hz, 1H), 8.78 (d, J2.4 Hz, 1H), 10.02 (s, 1H).

### Example 245—Synthesis of 6-(Hydroxymethyl)-N-(6-Methoxy-1-Methyl-1H-Indazol-7-yl)Pyridine-3-Sulfonamide (I-177)

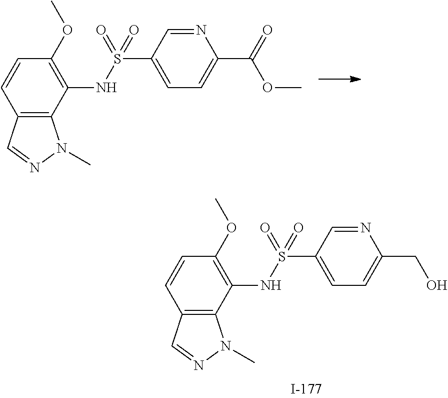 [SMILES: COC(=O)c1ccc(S(=O)(=O)Nc2c(OC)ccc3cnn(C)c23)cn1 ; COc1ccc2cnn(C)c2c1NS(=O)(=O)c1ccc(CO)nc1]

To a stirred solution of methyl 5-[(6-methoxy-1-methylindazol-7-yl)sulfamoyl] pyridine-2-carboxylate (120 mg, 0.319 mmol, 1 equiv.) in THF (4 mL) was added a solution of LiAlH₄ in THF (1.28 mL, 1.276 mmol, 4 equiv.) in portions at −20° C. under a nitrogen atmosphere. The resulting mixture was stirred for 2 h at −20° C. under a nitrogen atmosphere. The reaction was quenched by the addition of Na₂SO₄·10H₂O (20 mL in THF) at 0° C. The resulting mixture was filtered. The filtrate was concentrated under reduced pressure. The crude product (100 mg) was purified by prep-HPLC with the following conditions: column: XBridge Shield RP18 OBD, 30*150 mm, 5 m; mobile phase A: water(10 mmol/L NH₄HCO₃+0.05% NH₃H₂O), mobile phase B: ACN; flow rate 60 mL/min; gradient: 7% B to 23% B in 7 min; wavelength: 254 nm/220 nm; Rt1(min): 6.65; number of runs: 3) to afford 6-(hydroxymethyl)-N-(6-methoxy-1-methylindazol-7-yl) pyridine-3-sulfonamide (17.0 mg, 15.01%) as a white solid. LCMS: (ES, m/z): [M+H]⁺ 349; ¹H NMR (400 MHz, DMSO-d₆) δ 3.11 (s, 3H), 4.26 (s, 3H), 4.65 (d, J5.6 Hz, 2H), 5.64 (t, J5.6 Hz, 1H), 6.81 (d, J8.8 Hz, 1H), 7.65-7.68 (m, 2H), 7.98-8.02 (m, 2H), 8.65 (d, J 1.6 Hz, 1H), 9.90 (s, 1H).

### Example 246—Synthesis of 6-(2-Hydroxypropan-2-yl)-N-(6-Methoxy-1-Methyl-1H-Indazol-7-yl)Pyridine-3-Sulfonamide (I-178)

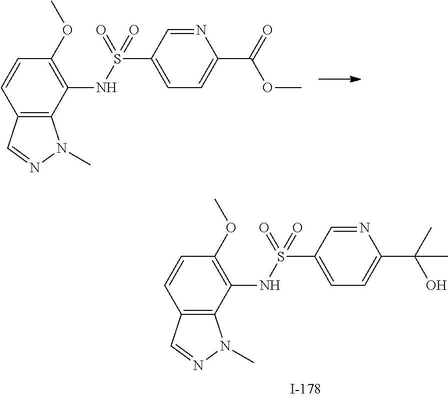 [SMILES: COC(=O)c1ccc(S(=O)(=O)Nc2c(OC)ccc3cnn(C)c23)cn1 ; COc1ccc2cnn(C)c2c1NS(=O)(=O)c1ccc(C(C)(C)O)nc1]

To a stirred solution of methyl 5-[(6-methoxy-1-methylindazol-7-yl)sulfamoyl] pyridine-2-carboxylate (100 mg, 0.266 mmol, 1 equiv.) in THE (2 mL) was added MeMgBr (0.30 mL, 0.902 mmol, 3.39 equiv.) dropwise at −78° C. under a nitrogen atmosphere. The resulting mixture was stirred for 1 h at room temperature under a nitrogen atmosphere. The reaction was quenched with sat. NH₄Cl (aq.) at room temperature. The resulting mixture was extracted with EtOAc (2×80 mL). The combined organic layers were washed with water (2×100 mL) and dried over anhydrous Na₂SO₄. After filtration, the filtrate was concentrated under reduced pressure. The crude product (100 mg) was purified by prep-HPLC with the following conditions: column: YMC-Actus Triart C18 ExRS, 19*250 mm, 5 m; mobile phase A: water (10 mmol/L NH₄HCO₃)+0.05% NH3. H₂O, mobile phase B: ACN; flow rate 60 mL/min; gradient: 22% B to 39% B in 7 min; wavelength: 254 nm/220 nm; Rt1(min): 9.48; number of runs: 4) to afford 6-(2-hydroxypropan-2-yl)-N-(6-methoxy-1-methylindazol-7-yl)pyridine-3-sulfonamide (16.5 mg, 16.42%) as a white solid. LCMS: (ES, m/z): [M+H]⁺ 377; ¹H NMR (400 MHz, DMSO-d₆) δ 1.47 (s, 6H), 3.08 (s, 3H), 4.26 (s, 3H), 5.44 (s, 1H), 6.80 (d, J 9.2 Hz, 1H), 7.67 (d, J8.8 Hz, 1H), 7.83 (d, J8.4 Hz, 1H), 7.96-7.98 (m, 2H), 8.66 (d, J2.4 Hz, 1H), 9.89 (s, 1H).

### Example 247—Synthesis of 7-Amino-2-Methyl-1-((6-(4-(Trifluoromethyl)-1H-Pyrazol-1-yl)Pyridin-3-yl)Sulfonyl)-1,2-Dihydro-3H-Indazol-3-One (I-179)

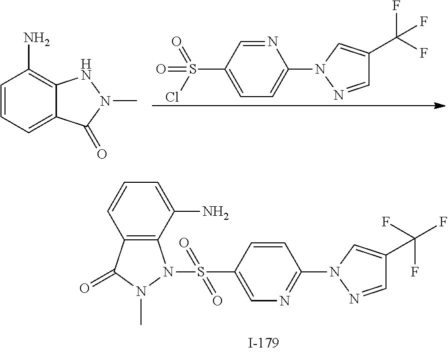

A solution of 7-amino-2-methyl-1H-indazol-3-one (200 mg, 1.226 mmol, 1 equiv.) in pyridine (4 mL) was treated with 6-[4-(trifluoromethyl)pyrazol-1-yl]pyridine-3-sulfonyl chloride (190.99 mg, 0.613 mmol, 0.5 equiv.) for 3 min at room temperature under a nitrogen atmosphere followed by the addition of DMAP (44.92 mg, 0.368 mmol, 0.3 equiv.) at room temperature. The resulting mixture was stirred for an additional 2 h at room temperature. The resulting mixture was concentrated under reduced pressure. The residue was purified by reversed-phase flash chromatography with the following conditions: column: C18 silica gel; mobile phase: MeCN in water (0.1% FA), 0% to 50% gradient in 20 min; detector: UV 254 nm. This resulted in 7-amino-2-methyl-1-((6-(4-(trifluoromethyl)-1H-pyrazol-1-yl)pyridin-3-yl)sulfonyl)-1,2-dihydro-3H-indazol-3-one (5.4 mg, 1.00%) as a white solid. LCMS: (ES, m/z): [M+H]⁺ 439; (400 MHz, DMSO-d₆): δ 4.10 (s, 3H), 5.35 (s, 2H), 6.60 (d, J 7.2 Hz, 1H), 6.66 (d, J8.0 Hz, 1H), 6.84 (d, J 7.6 Hz, 1H), 8.23 (d, J8.0 Hz, 1H), 8.47 (s, 1H), 8.60 (dd, J8.8, 2.4 Hz, 1H), 9.00 (d, J2.4 Hz, 1H), 9.36 (s, 1H).

### Example 248—Synthesis of N-(6-Methoxy-1-Methyl-1H-[1,2,3]Triazolo[4,5-C]Pyridin-7-yl)-6-(4-(Trifluoromethyl)-1H-Pyrazol-1-yl)Pyridine-3-Sulfonamide (I-180)

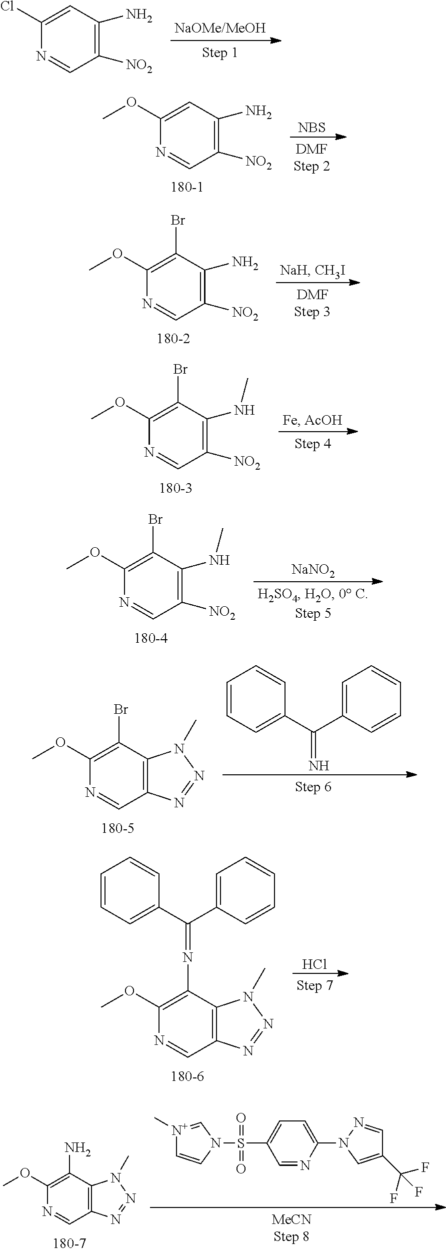

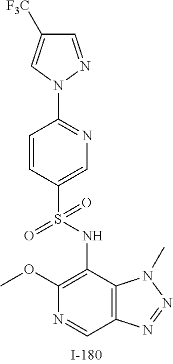

Step 1: Synthesis of 180-1. A solution of 2-chloro-5-nitropyridin-4-amine (1 g, 5.762 mmol, 1 equiv.) and NaOMe (3.11 g, 57.567 mmol, 9.99 equiv.) in MeOH (20 mL) was stirred for 4 h at 70° C. under a nitrogen atmosphere. The resulting mixture was diluted with water (50 mL). The resulting mixture was extracted with EtOAc (3×20 mL). The combined organic layers were washed with brine (50 mL) and dried over anhydrous Na₂SO₄. After filtration, the filtrate was concentrated under reduced pressure. This resulted in 2-methoxy-5-nitropyridin-4-amine (1.1 g, 95.94%) as a yellow solid.

Step 2: Synthesis of 180-2. A solution of 2-methoxy-5-nitropyridin-4-amine (3 g, 17.737 mmol, 1 equiv.) and NBS (3.79 g, 21.284 mmol, 1.2 equiv.) in DMF (20 mL) was stirred for 4 h at room temperature under a nitrogen atmosphere. The resulting mixture was diluted with water (100 mL). The resulting mixture was extracted with EtOAc (3×50 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 30 min; detector: UV 254 nm. This resulted in 3-bromo-2-methoxy-5-nitropyridin-4-amine (3.2 g, 58.19%) as a yellow solid.

Step 3: Synthesis of 180-3. To a stirred solution of 3-bromo-2-methoxy-5-nitropyridin-4-amine (3 g, 12.095 mmol, 1 equiv.) and NaH (580.51 mg, 24.190 mmol, 2 equiv.) in DMF (20 mL) was added CH₃I (1.51 mL, 24.190 mmol, 2 equiv.) dropwise at room temperature under a nitrogen atmosphere. The resulting mixture was stirred for 3 h at room temperature under a nitrogen atmosphere. The resulting mixture was diluted with water (50 mL). The resulting mixture was extracted with EtOAc (3×30 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% NH₃·H₂O+10 mmol/L NH₄HCO₃), 28% to 58% gradient in 30 min; detector: UV 254 nm. This resulted in 3-bromo-2-methoxy-N-methyl-5-nitropyridin-4-amine (850 mg, 24.14%) as a yellow solid.

Step 4: Synthesis of 180-4. A solution of 3-bromo-2-methoxy-N-methyl-5-nitropyridin-4-amine (150 mg, 0.572 mmol, 1 equiv.) and Fe (639.29 mg, 11.440 mmol, 20 equiv.) in AcOH (2 mL) was stirred for 4 h at room temperature under a nitrogen atmosphere. The resulting mixture was filtered and the filter cake was washed with EtOAc (3×10 mL). The filtrate was concentrated under reduced pressure. The residue was purified by prep-TLC (EA) to afford 5-bromo-6-methoxy-N4-methylpyridine-3,4-diamine (100 mg, 67.75%) as a brown oil.

Step 5: Synthesis of 180-5. To a stirred solution of 5-bromo-6-methoxy-N⁴-methylpyridine-3,4-diamine (90 mg, 0.388 mmol, 1 equiv.) and H₂SO₄ (76.06 mg, 0.776 mmol, 2 equiv.) in H₂O (3 mL) was added sodium nitrite (133.78 mg, 1.940 mmol, 5 equiv.) at 0° C. under a nitrogen atmosphere. The resulting mixture was stirred for 1 h at room temperature under a nitrogen atmosphere. The aqueous layer was extracted with EtOAc (3×3 mL). The combined organic layers were washed with brine (5 mL) and dried over anhydrous Na₂SO₄. After filtration, the filtrate was concentrated under reduced pressure. This resulted in 7-bromo-6-methoxy-1-methyl-[1,2,3]triazolo[4,5-c]pyridine (80 mg, 63.65%) as a dark yellow solid.

Step 6: Synthesis of 180-6. To a stirred solution of 7-bromo-6-methoxy-1-methyl-[1,2,3]triazolo[4,5-c]pyridine (250 mg, 1.029 mmol, 1 equiv.) and diphenylmethanimine (559.23 mg, 3.087 mmol, 3.00 equiv.) in DMF (5 mL) were added K₂CO₃ (426.45 mg, 3.086 mmol, 3.00 equiv.), Pd₂(dba)₃ (188.37 mg, 0.206 mmol, 0.2 equiv.) and Xantphos (297.57 mg, 0.514 mmol, 0.5 equiv.) at room temperature under a nitrogen atmosphere. The resulting mixture was stirred overnight at 140° C. under a nitrogen atmosphere. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 30 min; detector: UV 254 nm. This resulted in N-{6-methoxy-1-methyl-[1,2,3]triazolo[4,5-c]pyridin-7-yl}-1,1-diphenylmethanimine (200 mg, 50.96%) as a yellow solid.

Step 7: Synthesis of 180-7. A solution of N-{6-methoxy-1-methyl-[1,2,3]triazolo[4,5-c]pyridin-7-yl}-1,1-diphenylmethanimine (200 mg, 0.582 mmol, 1 equiv.) in HCl (gas) in 1,4-dioxane (5 mL) was stirred for 3 h at room temperature under a nitrogen atmosphere. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 30 min; detector: UV 254 nm. This resulted in 6-methoxy-1-methyl-[1,2,3]triazolo[4,5-c]pyridin-7-amine (65 mg, 56.06%) as a brown solid.

Step 8: Synthesis of N-(6-methoxy-1-methyl-11H-[1,2,3]triazolo[4,5-c]pyridin-7-yl)-6-(4-(trifluoromethyl)-1H-pyrazol-1-yl)pyridine-3-sulfonamide (1-180). A solution of 6-methoxy-1-methyl-[1,2,3]triazolo[4,5-c]pyridin-7-amine (50 mg, 0.279 mmol, 1 equiv.) and 1-methyl-3-{6-[4-(trifluoromethyl)pyrazol-1-yl]pyridin-3-ylsulfonyl}imidazol-1-ium (149.98 mg, 0.419 mmol, 1.5 equiv.) in acetonitrile (2 mL) was stirred for 2 h at 80° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. This resulted in N-{6-methoxy-1-methyl-[1,2,3]triazolo[4,5-c]pyridin-7-yl}-6-[4-(trifluoromethyl)pyrazol-1-yl]pyridine-3-sulfonamide (17.2 mg, 13.55%) as a white solid. LCMS: (ES, m/z): [M+H]⁺ 455; ¹H NMR (400 MHz, DMSO-d₆) δ 3.35 (s, 3H), 4.47 (s, 3H), 8.18 (d, J 8.8 Hz, 1H), 8.30 (dd, J 8.8, 2.4 Hz, 1H), 8.42 (s, 1H), 8.70 (d, J 2.4 Hz, 1H), 9.10 (s, 1H), 9.33 (s, 1H), 10.55 (s, 1H).

### Example 249—Synthesis of N-(2-Methyl-3-Oxo-2,3-Dihydro-1H-Indazol-7-yl)-6-(4-(Trifluoromethyl)-1H-Pyrazol-1-yl)Pyridine-3-Sulfonamide (I-181)

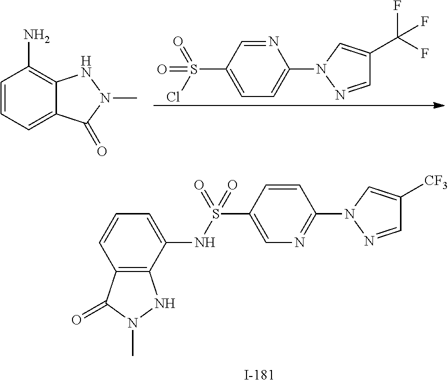

A solution of 7-amino-2-methyl-1H-indazol-3-one (100 mg, 0.613 mmol, 1 equiv.) in pyridine (2 mL) was treated with 6-[4-(trifluoromethyl)pyrazol-1-yl]pyridine-3-sulfonyl chloride (286.49 mg, 0.919 mmol, 1.5 equiv.) for 3 min at room temperature under a nitrogen atmosphere followed by the addition of DMAP (7.49 mg, 0.061 mmol, 0.1 equiv.) at room temperature. The resulting mixture was stirred for an additional 2 h at room temperature. The resulting mixture was concentrated under reduced pressure. The residue was purified by reversed-phase flash chromatography with the following conditions: column: C18 silica gel; mobile phase: MeCN in water (10 mmol/L NH₄HCO₃), 0% to 100% gradient in 20 min; detector: UV 254 nm. This resulted in N-(2-methyl-3-oxo-2,3-dihydro-1H-indazol-7-yl)-6-(4-(trifluoromethyl)-1H-pyrazol-1-yl)pyridine-3-sulfonamide (3.1 mg, 1.11%) as a white solid. LCMS: (ES, m/z): [M+H]⁺ 439; ¹H NMR (400 MHz, DMSO-d₆): δ 3.99 (s, 3H), 6.49 (d, J5.1 Hz, 1H), 6.77 (t, J7.6 Hz, 1H), 7.48 (d, J7.6 Hz, 1H), 8.19 (d, J8.8 Hz, 1H), 8.30 (dd, J8.8, 2.4 Hz, 1H), 8.42 (s, 1H), 8.66 (d, J2.0 Hz, 1H), 9.33 (s, 1H), 10.27 (s, 1H), 10.74 (s, 1H).

### Example 250—Synthesis of N-(1,2-Dimethyl-3-Oxo-2,3-Dihydro-1H-Indazol-4-yl)-6-(4-(Trifluoromethyl)-1H-Pyrazol-1-yl)Pyridine-3-Sulfonamide (I-182)

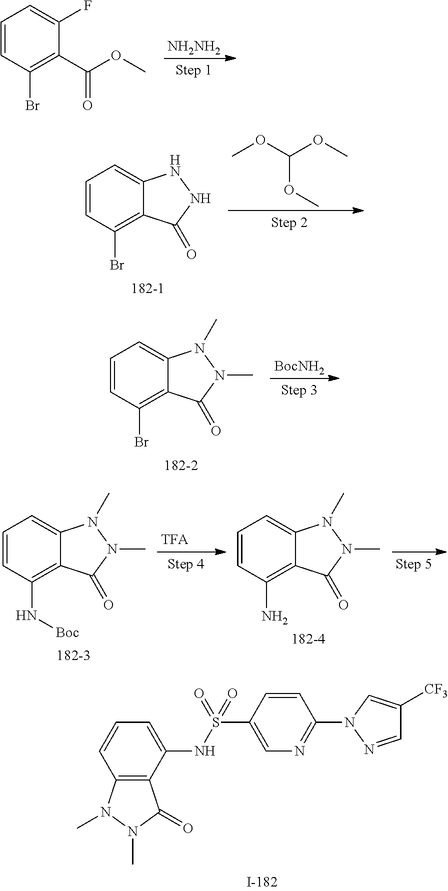

Step 1: Synthesis of 182-1. To a stirred solution of methyl 2-bromo-6-fluorobenzoate (5 g, 21.456 mmol, 1 equiv.) in EtOH (50 mL) was added hydrazine hydrate (10.74 g, 214.560 mmol, 10 equiv.) dropwise at room temperature under a nitrogen atmosphere. The resulting mixture was stirred for 16 h at 80° C. under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 10% to 12% gradient in 15 min; detector: UV 220 nm. This resulted in 182-1 (2.63 g, 57.54%) as a brown solid.

Step 2: Synthesis of 182-2. To a stirred solution of 182-1 (1.42 g, 6.666 mmol, 1 equiv.) and trimethyl orthoformate (2.48 g, 23.331 mmol, 3.5 equiv.) in toluene (14 mL) was added H₂SO₄ (391 uL, 7.336 mmol, 1.10 equiv.) dropwise at room temperature under a nitrogen atmosphere. The resulting mixture was stirred for 4 h at 110° C. under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 10% to 20% gradient in 10 min; detector: UV 220 nm. This resulted in 182-2 (400 mg, 36.89%) as a brown solid.

Step 3: Synthesis of 182-3. To a stirred solution of 182-2 (290 mg, 1.203 mmol, 1 equiv.) and BocNH₂ (704.58 mg, 6.015 mmol, 5 equiv.) in dioxane (15 mL) were added Pd-PEPPSI-Ipent (380.9 mg, 0.481 mmol, 0.4 equiv.) and t-BuOK (539.92 mg, 4.812 mmol, 4 equiv.) in portions at room temperature under a nitrogen atmosphere. The resulting mixture was stirred for 12 h at 100° C. under a nitrogen atmosphere. The reaction was quenched with water (20 mL). The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. The resulting mixture was concentrated under reduced pressure. This resulted in 182-3 (160 mg, 47.96%) as a white solid.

Step 4: Synthesis of 182-4. To a stirred solution of 182-3 (160 mg, 0.577 mmol, 1 equiv.) in DCM (2 mL) was added TFA (0.5 mL) dropwise at room temperature under a nitrogen atmosphere. The resulting mixture was stirred for 2 h at room temperature under a nitrogen atmosphere. The reaction was diluted with water (5 mL). The resulting mixture was extracted with DCM (2×5 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 50% to 60% gradient in 10 min; detector: UV 254 nm. The resulting mixture was concentrated under reduced pressure. This resulted in 182-4 (100 mg, 97.81%) as a white solid.

Step 5: Synthesis of N-(1,2-dimethyl-3-oxo-2,3-dihydro-1H-indazol-4-yl)-6-(4-(trifluoromethyl)-1H-pyrazol-1-yl)pyridine-3-sulfonamide (I-182). To a stirred solution of 182-4 (100 mg, 0.564 mmol, 1 equiv.) in MeCN (5 mL) was added 1-methyl-3-{6-[4-(trifluoromethyl) pyrazol-1-yl] pyridin-3-ylsulfonyl}imidazol-1-ium triflate (343.58 mg, 0.677 mmol, 1.2 equiv.) in portions at room temperature under a nitrogen atmosphere. The resulting mixture was stirred for 2 h at 50° C. under a nitrogen atmosphere. The resulting mixture was extracted with EtOAc (2×10 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. The resulting mixture was concentrated under reduced pressure. This resulted in N-(1,2-dimethyl-3-oxo-2,3-dihydro-1H-indazol-4-yl)-6-(4-(trifluoromethyl)-1H-pyrazol-1-yl)pyridine-3-sulfonamide (30.4 mg, 11.85%) as a white solid. LCMS: (ES, m/z): [M+H]⁺ 453; ¹H NMR (400 MHz, DMSO-d₆): δ 3.28 (s, 3H), 3.30 (s, 3H), 7.00 (d, J7.6 Hz, 1H), 7.18 (d, J8.4 Hz, 1H), 7.47 (t, J8.0 Hz, 1H), 8.13 (d, J8.8 Hz, 1H), 8.39 (s, 1H), 8.55 (d, J8.8, 2.4 Hz, 1H), 9.01 (d, J 1.6 Hz, 1H), 9.27 (s, 1H), 9.97 (s, 1H).

### Example 251—Synthesis of N-(6-Methoxy-1-Methyl-1H-Pyrazolo[4,3-C]Pyridin-7-yl)-N-Methyl-6-(4-(Trifluoromethyl)-1H-Pyrazol-1-yl)Pyridine-3-Sulfonamide (I-183)

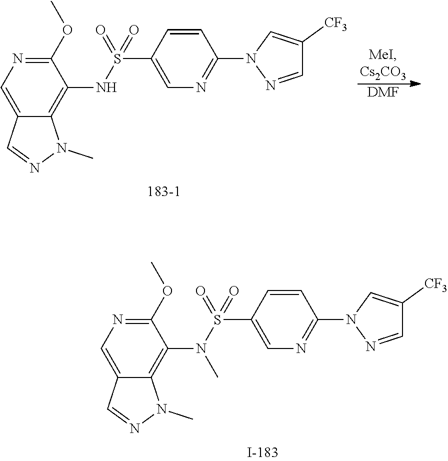

A solution of 183-1 (90.6 mg, 0.200 mmol, 1 equiv.) in DMF (1 mL) was treated with Cs₂CO₃ (130.21 mg, 0.400 mmol, 2 equiv.) for 5 min at room temperature followed by the addition of Mel (34.04 mg, 0.240 mmol, 1.2 equiv.) dropwise at room temperature. Then the mixture was stirred at room temperature for 3 h. After the reaction was completed, the solution was quenched with water (15 mL) and then extracted with EtOAc (4×20 mL). The organic phase was combined, washed with the brine (20 mL) and concentrated under vacuum. The residue was purified by prep-HPLC with the following conditions: column: XBridge Shield RP₁₈ OBD, 30*150 mm, 5 μm; mobile phase A: water (10 mmol/L NH₄HCO₃), mobile phase B: ACN; flow rate 60 mL/min; gradient: 42% B to 70% B in 7 min; wavelength: 254 nm/220 nm; Rt1(min): 6.64) to afford N-(6-methoxy-1-methyl-1H-pyrazolo[4,3-c]pyridin-7-yl)-N-methyl-6-(4-(trifluoromethyl)-1H-pyrazol-1-yl)pyridine-3-sulfonamide (44.5 mg, 47.60%) as a purple solid. LCMS: (ES, m/z): [M+H]⁺ 468; ¹H NMR (400 MHz, DMSO-d₆) δ 3.33 (s, 3H), 3.36 (s, 3H), 4.22 (s, 3H), 8.22 (d, J 8.8 Hz,1H), 8.27 (s, 1H), 8.35 (dd, J8.8, 2.4 Hz, 1H), 8.44 (s, 1H), 8.75 (s, 1H), 8.77 (d, J2.4 Hz, 1H), 9.35 (s, 1H).

### Example 252—Synthesis of N-(6-Methoxy-1-Methyl-1H-Pyrazolo[4,3-C]Pyridin-7-yl)-6-(4-Methyl-1H-Pyrazol-1-yl)Pyridine-3-Sulfonamide (I-184)

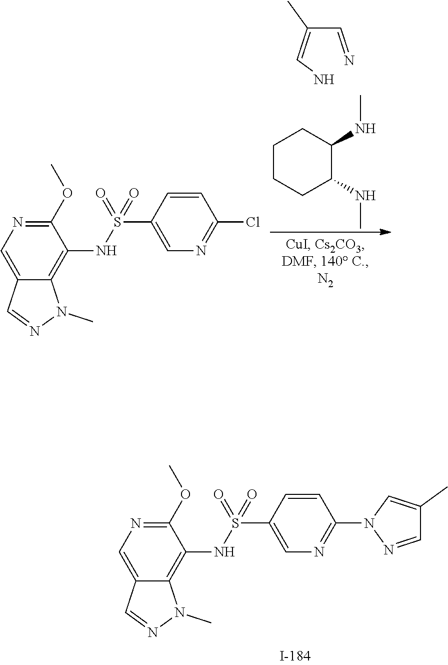

A solution of 6-chloro-N-(6-methoxy-1-methyl-1H-pyrazolo[4,3-c]pyridin-7-yl)pyridine-3-sulfonamide (100 mg, 0.283 mmol, 1.00 equiv.), (1R,2R)—N1,N2-dimethylcyclohexane-1,2-diamine (8.04 mg, 0.057 mmol, 0.20 equiv.), 4-methyl-1H-pyrazole (30.1 mg, 0.368 mmol, 1.30 equiv.), CuI (5.3 mg, 0.028 mmol, 0.10 equiv.) and Cs₂CO₃ (276.2 mg, 0.849 mmol, 3.00 equiv.) in DMF (5 mL) was stirred for 2 h at 140° C. under a nitrogen atmosphere. The mixture was allowed to cool down to room temperature. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃)*0.05% NH₃·H₂O, mobile phase B: ACN; flow rate 60 mL/min; gradient: 13% B to 40% B in7 min; wavelength: 254 nm/220 nm; Rt1(min): 8.43 to afford N-{6-methoxy-1-methylpyrazolo[4,3-c]pyridin-7-yl}-6-(4-methylpyrazol-1-yl)pyridine-3-sulfonamide (17.9 mg, 15.49%) as an off-white solid. LCMS: (ES, m/z): [M+H]⁺ 400; ¹H NMR (400 MHz, DMSO-d₆) δ 2.12 (s, 3H), 3.27 (s, 3H), 4.24 (s, 3H), 7.76 (s, 1H), 8.03 (d, J 8.8 Hz, 1H), 8.16 (dd, J 8.8, 2.4 Hz, 1H), 8.22 (s, 1H), 8.48 (s, 1H), 8.58 (d, J=2.4 Hz, 1H), 8.63 (s, 1H). 10.09 (s, 1H).

### Example 253—Synthesis of 6-(3-Cyclopropyl-1H-Pyrazol-1-yl)-N-(6-Methoxy-1-Methyl-1H-Pyrazolo[4,3-C]Pyridin-7-yl)Pyridine-3-Sulfonamide (I-185)

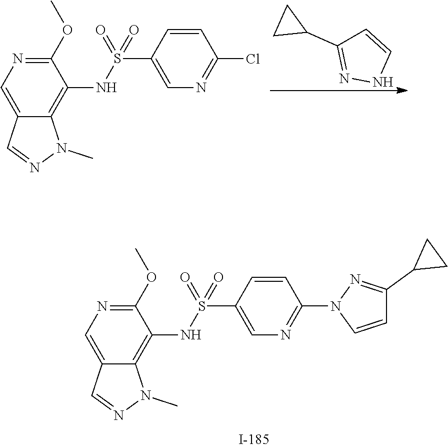

A solution of 6-chloro-N-(6-methoxy-1-methyl-1H-pyrazolo[4,3-c]pyridin-7-yl)pyridine-3-sulfonamide (80 mg, 0.226 mmol, 1 equiv.), 3-cyclopropyl-1H-pyrazole (40 mg, 0.370 mmol, 1.64 equiv.) and Cs₂CO₃ (150 mg, 0.460 mmol, 2.04 equiv.) in DMSO (3 mL) was stirred for 3 h at 140° C. under a nitrogen atmosphere. The resulting mixture was diluted with water (20 mL). The resulting mixture was extracted with EtOAc (3×50 mL). The combined organic layers were washed with brine (3×5 mL) and dried over anhydrous Na₂SO₄. After filtration, the filtrate was concentrated under reduced pressure. The crude product (150 mg) was purified by prep-HPLC with the following conditions: column: XBridge Prep OBD C18, 30*150 mm, 5 m; mobile phase A: water (10 mmol/L NH₄HCO₃)+0.05% NH3. H₂O, mobile phase B: ACN; flow rate 60 mL/min; gradient: 21% B to 48% B in7 min; wavelength: 254 nm/220 nm; Rt1(min): 8.93) to afford 6-(3-cyclopropyl-1H-pyrazol-1-yl)-N-(6-methoxy-1-methyl-1H-pyrazolo[4,3-c]pyridin-7-yl)pyridine-3-sulfonamide (30 mg, 31.06%) as an off-white solid. LCMS: (ES, m/z): [M+H]⁺ 426; ¹H NMR (400 MHz, DMSO-d₆) δ 0.76-0.84 (m, 2H), 0.92-1.03 (m, 2H), 1.97-2.10 (m, 1H), 3.28 (s, 3H), 4.24 (s, 3H), 6.37 (d, J2.8 Hz, 1H), 7.99 (d, J 8.4 Hz, 1H), 8.15 (dd, J8.8, 2.4 Hz, 1H), 8.22 (s, 1H), 8.52-8.59 (m, 2H), 8.62 (s, 1H), 9.98 (s, 1H).

### Example 254—Synthesis of N-(6-Methoxy-1-Methyl-1H-Pyrazolo[4,3-C]Pyridin-7-yl)-6-(2-Methylthiazol-4-yl)Pyridine-3-Sulfonamide (I-186)

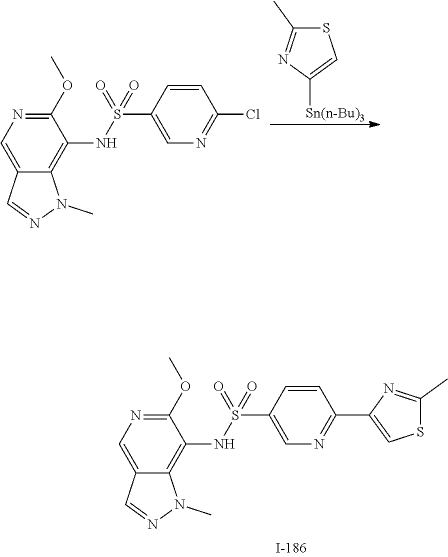

To a stirred mixture of 6-chloro-N-(6-methoxy-1-methyl-1H-pyrazolo[4,3-c]pyridin-7-yl)pyridine-3-sulfonamide (50 mg, 0.141 mmol, 1 equiv.) and 2-methyl-4-(tributylstannyl)thiazole (54.86 mg, 0.141 mmol, 1 equiv.) in DMF (8 mL) were added XPhos Pd G3 (11.96 mg, 0.014 mmol, 0.1 equiv.), ZnCl2 (57.78 mg, 0.423 mmol, 3 equiv.) and XPhos (13.48 mg, 0.028 mmol, 0.2 equiv.) in portions at room temperature under a nitrogen atmosphere. The resulting mixture was stirred for an additional 2 h at 90° C. The reaction was quenched by the addition of water (20 mL) at room temperature. The aqueous layer was extracted with EtOAc (3×120 mL). The crude product (150 mg) was purified by prep-HPLC with the following conditions: column: XBridge Prep OBD C18, 30*150 mm, 5 m; mobile phase A: water (10 mmol/L NH₄HCO₃+0.05% NH₃. H₂O), mobile phase B: ACN; flow rate 60 mL/min; gradient: 15% B to 43% B in 7 min; wavelength: 254 nm/220 nm; Rt1(min): 8.12) to afford N-(6-methoxy-1-methyl-1H-pyrazolo[4,3-c]pyridin-7-yl)-6-(2-methylthiazol-4-yl)pyridine-3-sulfonamide (6.7 mg, 11.37%) as a white solid. LCMS: (ES, m/z): [M+H]⁺ 417; ¹H NMR (400 MHz, DMSO-d₆) δ 2.76 (s, 3H), 3.22 (s, 3H), 4.25 (s, 3H), 8.10 (d, J8.0 Hz, 1H), 8.20-8.23 (m, 2H), 8.33 (s, 1H), 8.64 (s, 1H), 8.76 (s, 1H), 10.14 (s, 1H).

### Example 255—Synthesis of 6-(4-Cyclopropyl-1H-Pyrazol-1-yl)-N-(6-Methoxy-1-Methyl-1H-Pyrazolo[4,3-C]Pyridin-7-yl)Pyridine-3-Sulfonamide (I-187)

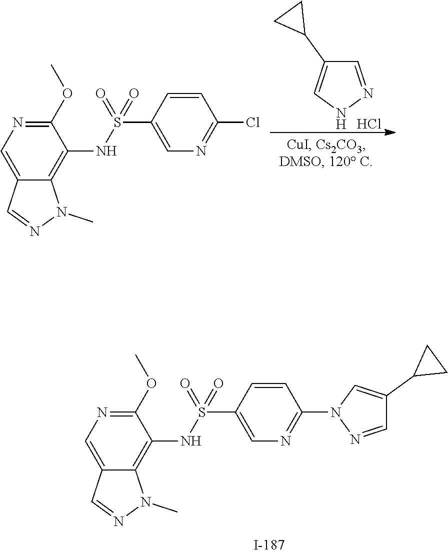 [SMILES: COc1ncc2cnn(C)c2c1NS(=O)(=O)c1ccc(Cl)nc1 ; c1n[nH]cc1C1CC1 ; Cl ; COc1ncc2cnn(C)c2c1NS(=O)(=O)c1ccc(-n2cc(C3CC3)cn2)nc1]

A mixture of 6-chloro-N-{6-methoxy-1-methylpyrazolo[4,3-c]pyridin-7-yl}pyridine-3-sulfonamide (400 mg, 1.131 mmol, 0.82 equiv.), 4-cyclopropyl-1H-pyrazole hydrochloride (200 mg, 1.383 mmol, 1.00 equiv.), CuI (263.42 mg, 1.383 mmol, 1 equiv.), (1R,2R)-1-N,1-N-dimethylcyclohexane-1,2-diamine (98.37 mg, 0.692 mmol, 0.5 equiv.) and Cs₂CO₃ (1351.95 mg, 4.149 mmol, 3 equiv.) in DMSO (10 mL) was stirred for 12 h at 120° C. under a nitrogen atmosphere. The resulting mixture was diluted with water (30 mL). The resulting mixture was extracted with EtOAc (3×50 mL). The combined organic layers were washed with brine (10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm to afford 6-(4-cyclopropylpyrazol-1-yl)-N-{6-methoxy-1-methylpyrazolo[4,3-c]pyridin-7-yl}pyridine-3-sulfonamide (139 mg, 23.45%) as a yellow solid. LCMS: (ES, m/z): [M+H]⁺ 426; ¹H NMR (400 MHz, DMSO-d₆) δ 0.62-0.70 (m, 2H), 0.85-0.94 (m, 2H), 1.79-1.88 (m, 1H), 3.28 (s, 3H), 4.24 (s, 3H), 7.76 (s, 1H), 8.04 (d, J 8.8 Hz, 1H), 8.17 (dd, J 8.8, 2.4 Hz, 1H), 8.24 (s, 1H), 8.45 (s, 1H), 8.57 (d, J2.0 Hz, 1H), 8.66 (s, 1H), 10.14 (s, 1H).

### Example 256—Synthesis of N-(6-Methoxy-1-Methyl-1H-Indol-7-yl)-6-(4-(Trifluoromethyl)-1H-Pyrazol-1-yl)Pyridine-3-Sulfonamide (I-188)

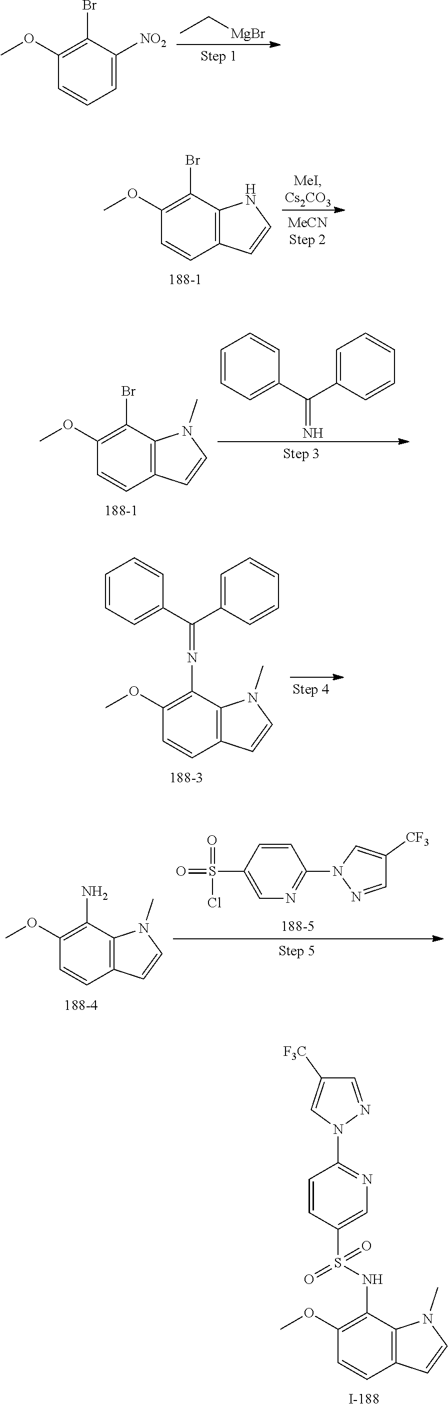

Step 1: Synthesis of 188-1. To a stirred solution of 2-bromo-1-methoxy-3-nitrobenzene (2 g, 8.619 mmol, 1 equiv.) in THE (20 mL) was added bromo(ethenyl)magnesium (17 mL, 34.476 mmol, 4 equiv., 2 M) dropwise over 20 min at −45° C. under a nitrogen atmosphere. The resulting mixture was stirred for 2 h at −40° C. under a nitrogen atmosphere. The reaction was quenched with sat. NH₄Cl (aq.) (50 mL) at −45° C. The resulting mixture was extracted with EtOAc (3×50 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 7-bromo-6-methoxy-1H-indole (2 g, 78.00%) as a yellow solid.

Step 2: Synthesis of 188-2. A solution of 7-bromo-6-methoxy-1H-indole (300 mg, 1.327 mmol, 1 equiv.); Mel (207.19 mg, 1.460 mmol, 1.1 equiv.) and Cs₂CO₃ (1297.09 mg, 3.981 mmol, 3 equiv.) in acetonitrile (2 mL) was stirred for 2 h at room temperature under an air atmosphere. The resulting mixture was diluted with water (10 mL). The resulting mixture was extracted with CH₂Cl₂ (3×10 mL). The combined organic layers were washed with water (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 7-bromo-6-methoxy-1-methylindole (140 mg, 41.30%) as a yellow oil.

Step 3: Synthesis of 188-3. A mixture of 7-bromo-6-methoxy-1-methylindole (120 mg, 0.500 mmol, 1 equiv.), diphenylmethanimine (135.87 mg, 0.750 mmol, 1.5 equiv.), Pd(dba)₂ (57.48 mg, 0.100 mmol, 0.2 equiv.), XantPhos (72.30 mg, 0.125 mmol, 0.25 equiv.) and K₂CO₃ (207.22 mg, 1.500 mmol, 3 equiv.) in DMF (2 mL) was stirred for 16 h at 140° C. under a nitrogen atmosphere. The mixture was allowed to cool down to room temperature. The resulting mixture was diluted with water (10 mL). The resulting mixture was extracted with EtOAc (3×10 mL). The combined organic layers were washed with brine (30 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in N-(6-methoxy-1-methylindol-7-yl)-1,1-diphenylmethanimine (110 mg, 57.54%) as a yellow oil.

Step 4: Synthesis of 188-4. A solution of N-(6-methoxy-1-methylindol-7-yl)-1,1-diphenylmethanimine (90 mg, 0.264 mmol, 1 equiv.), AcONa (32.53 mg, 0.396 mmol, 1.5 equiv.) and hydroxylamine hydrochloride (27.56 mg, 0.396 mmol, 1.5 equiv.) in MeOH (1.5 mL) was stirred for 2 h at 50° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. This resulted in 6-methoxy-1-methylindol-7-amine (50 mg, 81.57%) as a yellow oil. The crude product was used in the next step directly without further purification.

Step 5: Synthesis of N-(6-methoxy-1-methyl-1H-indol-7-yl)-6-(4-(trifluoromethyl)-1H-pyrazol-1-yl)pyridine-3-sulfonamide (I-188). A solution of 6-methoxy-1-methylindol-7-amine (50 mg, 0.284 mmol, 1 equiv.) and 6-[4-(trifluoromethyl)pyrazol-1-yl]pyridine-3-sulfonyl chloride (132.64 mg, 0.426 mmol, 1.5 equiv.) in pyridine (1 mL) was stirred for 16 h at room temperature under an air atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in N-(6-methoxy-1-methylindol-7-yl)-6-[4-(trifluoromethyl)pyrazol-1-yl]pyridine-3-sulfonamide (13.2 mg, 10.14%) as a white solid. LCMS: (ES, m/z): [M+H]⁺ 452; ¹H NMR (400 MHz, DMSO-d₆) δ 3.09 (s, 3H), 4.07 (s, 3H), 6.37 (d, J3.2 Hz, 1H), 6.65 (d, J8.8 Hz, 1H), 7.19 (d, J3.2 Hz, 1H), 7.43 (d, J8.8 Hz, 1H), 8.18 (d, J 8.8 Hz, 1H), 8.27 (dd, J8.8, 2.4 Hz, 1H), 8.41 (s, 1H), 8.66 (d, J2.0 Hz, 1H), 9.34 (s, 1H), 9.85 (s, 1H).

### Example 257—Synthesis of N-(1,6-Dimethyl-1H-Pyrazolo[4,3-C]Pyridin-7-yl)-6-(4-(Trifluoromethyl)-1H-Pyrazol-1-yl)Pyridine-3-Sulfonamide (I-189)

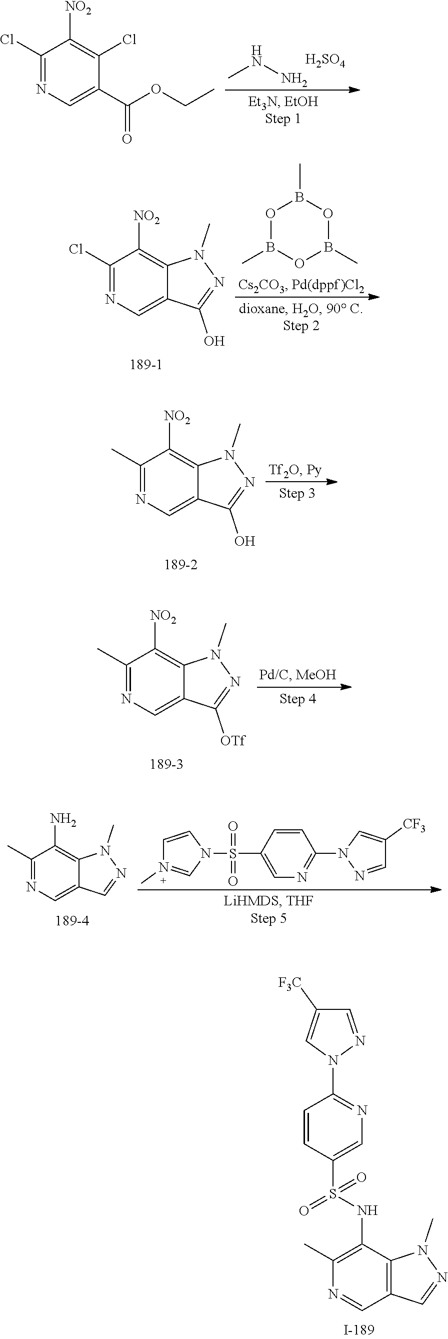

Step 1: Synthesis of 189-1. A solution of ethyl 4,6-dichloro-5-nitropyridine-3-carboxylate (1.056 g, 3.98 mmol, 1 equiv.), Et₃N (1209.51 mg, 11.95 mmol, 3 equiv.) and methyl hydrazine sulfuric acid salt (631.75 mg, 4.38 mmol, 1.1 equiv.) in EtOH (20 mL) was stirred overnight at room temperature. After the reaction was complete, the solution was concentrated under vacuum. The residue was purified by reversed-phase flash chromatography with the following conditions: column: C18 silica gel; mobile phase: ACN in water, 0% to 100% gradient in 20 min; detector: UV 254 nm to give 189-1 (449 mg, 49.30%) as a red solid.

Step 2: Synthesis of 189-2. A solution of 189-1 (930 mg, 4.068 mmol, 1 equiv.), trimethyl-1,3,5,2,4,6-trioxatriborinane (766.05 mg, 6.102 mmol, 1.5 equiv.), Pd(dppf)Cl₂ (595.38 mg, 0.813 mmol, 0.2 equiv.) and Cs₂CO₃ (2.65 g, 8.14 mmol, 2 equiv.) in dioxane (45 mL) was stirred overnight at 90° C. under a nitrogen atmosphere. After the reaction was completed, the resulting mixture was diluted with water (100 mL). The resulting mixture was extracted with EtOAc (4×100 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water (0.1% NH₃. H₂O), 0% to 100% gradient in 35 min; detector: UV 254 nm to give 189-2 (780 mg, 92.10%) as a yellow solid.

Step 3: Synthesis of 189-3. A solution of 189-2 (423 mg, 4.07 mmol, 1 equiv.), pyridine (1.284 g, 15.444 mmol, 4 equiv.) and Tf₂O (2.864 g, 10.16 mmol, 2.5 equiv.) in DCM (40 mL) was stirred overnight at room temperature under a nitrogen atmosphere. After the reaction was complete, the resulting mixture was diluted with water (50 mL). The resulting mixture was extracted with EtOAc (4×60 mL). The combined organic layers were washed with brine (100 mL) and dried over anhydrous Na₂SO₄. After filtration, the filtrate was concentrated under reduced pressure. The residue was purified by prep-TLC (PE/EA 5:1) to afford 189-3 (465 mg, 33.63%) as a light-yellow solid.

Step 4: Synthesis of 189-4. A mixture of 189-3 (465 mg, 1.368 mmol, 1 equiv.) and Pd/C (150 mg, 1.41 mmol, 1.03 equiv.) in MeOH (60 mL) was stirred overnight at room temperature under a hydrogen atmosphere. After the reaction was complete, the solid was filtered. The filtrate was concentrated under vacuum to give 189-4 (432 mg, 97.44%) as a light-yellow solid.

Step 5: Synthesis of N-(1,6-dimethyl-1H-pyrazolo[4,3-c]pyridin-7-yl)-6-(4-(trifluoromethyl)-1H-pyrazol-1-yl)pyridine-3-sulfonamide (1-189). A solution of 189-4 (162 mg, 0.999 mmol, 1 equiv.) in THE (10 mL) was treated with LiHMDS (417.82 mg, 2.498 mmol, 2.5 equiv.) for 10 min at −30° C. followed by the addition of 3-methyl-1-{6-[4-(trifluoromethyl)pyrazol-1-yl]pyridin-3-ylsulfonyl}imidazol-1-ium (429.46 mg, 1.199 mmol, 1.20 equiv.) in portions at −10° C. Then the reaction was stirred from −10° C. to room temperature for 3 h. After the reaction was complete, the resulting mixture was quenched with water (50 mL). The resulting mixture was extracted with EtOAc (4×60 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water, 0% to 100% gradient in 30 min; detector: UV 254 nm to give the crude product. The crude product was purified again by prep-HPLC with the following conditions: column: Xselect CSH C₁₈ OBD, 30*150 mm 5 μm; mobile phase A: water (0.1% FA), mobile phase B: ACN; flow rate 60 mL/min; gradient: 23% B to 34% B in 7 min; wavelength: 254 nm/220 nm; Rt1(min): 6.3) to afford N-(1,6-dimethyl-1H-pyrazolo[4,3-c]pyridin-7-yl)-6-(4-(trifluoromethyl)-1H-pyrazol-1-yl)pyridine-3-sulfonamide (31.9 mg, 7.28%) as a white solid. LCMS: (ES, m/z): [M+H]⁺ 438; ¹H NMR (400 MHz, DMSO-d₆) δ 1.86 (s, 3H), 4.25 (s, 3H), 8.17-8.28 (m, 3H), 8.42 (s, 1H), 8.67 (d, J 1.6 Hz, 1H), 8.87 (s, 1H), 9.32 (s, 1H), 10.63 (s, 1H).

### Example 258—Synthesis of N-(2-Methyl-3-Oxo-2,3-Dihydro-1H-Indazol-4-yl)-6-(4-(Trifluoromethyl)-1H-Pyrazol-1-yl)Pyridine-3-Sulfonamide (I-190)

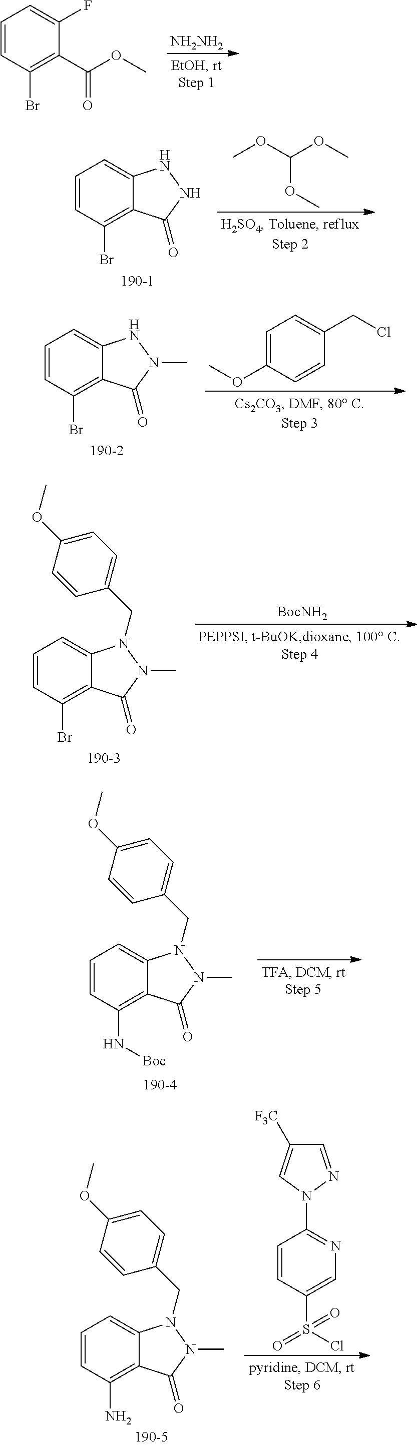

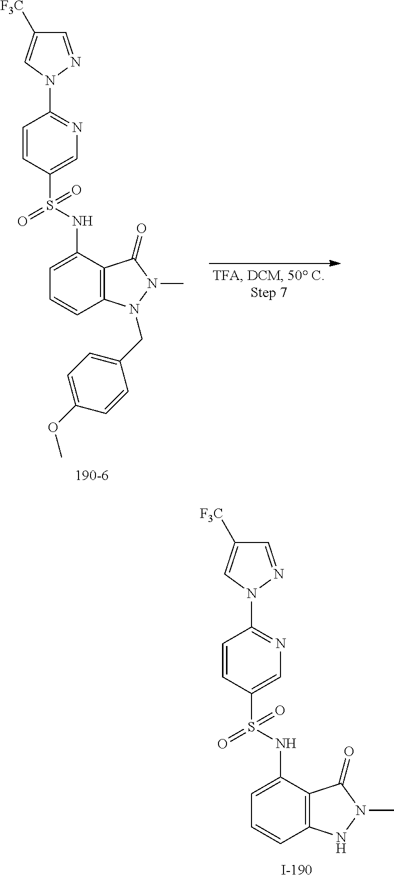 [SMILES: COc1ccc(Cn2c3cccc(NS(=O)(=O)c4ccc(-n5cc(C(F)(F)F)cn5)nc4)c3c(=O)n2C)cc1 ; Cn1[nH]c2cccc(NS(=O)(=O)c3ccc(-n4cc(C(F)(F)F)cn4)nc3)c2c1=O]

Step 1: Synthesis of 190-1. To a stirred solution of methyl 2-bromo-6-fluorobenzoate (5 g, 21.456 mmol, 1 equiv.) in EtOH (50 mL) was added hydrazine hydrate (10.74 g, 214.560 mmol, 10 equiv.) dropwise at room temperature under a nitrogen atmosphere. The resulting mixture was stirred for 16 h at 80° C. under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 10% to 12% gradient in 15 min; detector: UV 220 nm. This resulted in 190-1 (2.63 g, 57.54%) as a brown solid.

Step 2: Synthesis of 190-2. To a stirred solution of 190-1 (1.42 g, 6.666 mmol, 1 equiv.) and trimethyl orthoformate (2.48 g, 23.331 mmol, 3.5 equiv.) in toluene (14 mL) was added H₂SO₄ (391 uL, 7.336 mmol, 1.10 equiv.) dropwise at room temperature under a nitrogen atmosphere. The resulting mixture was stirred for 4 h at 110° C. under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 10% to 20% gradient in 10 min; detector: UV 220 nm. This resulted in 190-2 (870 mg, 57.48%) as a brown solid.

Step 3: Synthesis of 190-3. To a stirred solution of 190-2 (740 mg, 3.259 mmol, 1 equiv.) and Cs₂CO₃ (3.19 g, 9.777 mmol, 3.00 equiv.) in DMF (18 mL) was added 4-methoxybenzyl chloride (920 uL, 6.515 mmol, 2.00 equiv.) dropwise at room temperature under a nitrogen atmosphere. The resulting mixture was stirred for 3 h at 80° C. under a nitrogen atmosphere. The precipitated solids were collected by filtration and washed with MeCN (3×18 mL). The filtrate was concentrated under reduced pressure. The residue was purified by reversed-phase flash chromatography with the following conditions: column: C18 silica gel; mobile phase: MeCN in water (10 mmol/L NH₄HCO₃), 45% to 47% gradient in 30 min; detector: UV 210 nm. This resulted in 190-3 (776 mg, 68.58%) as a white solid.

Step 4: Synthesis of 190-4. To a stirred solution of 190-3 (490 mg, 1.411 mmol, 1 equiv.), t-BuOK (475.1 mg, 4.233 mmol, 3 equiv.) and {1,3-bis[2,6-bis(pentan-3-yl)phenyl]-4,5-dichloro-2,3-dihydro-1H-imidazol-2-yl}dichloro(2-methyl-1lambda4-pyridin-1-yl)palladium (237.4 mg, 0.282 mmol, 0.2 equiv.) in dioxane (24 mL) was added tert-butyl carbamate (245 mg, 2.117 mmol, 1.5 equiv.) in portions at room temperature under a nitrogen atmosphere. The resulting mixture was stirred for 3 h at 100° C. under a nitrogen atmosphere. The resulting mixture was filtered and the filter cake was washed with MeCN (3×8 mL). The filtrate was concentrated under reduced pressure. The residue was purified by reversed-phase flash chromatography with the following conditions: column: C18 silica gel; mobile phase: MeCN in water (10 mmol/L NH₄HCO₃), 60% to 65% gradient in 10 min; detector: UV 220 nm. This resulted in 190-4 (260 mg, 48.05%) as a brown solid.

Step 5: Synthesis of 190-5. To a stirred solution of 190-4 (260 mg, 0.678 mmol, 1 equiv.) in DCM (13 mL) was added TFA (2.6 mL, 35.004 mmol, 51.62 equiv.) dropwise at room temperature under a nitrogen atmosphere. The resulting mixture was stirred for 1 h at room temperature under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 48% to 50% gradient in 20 min; detector: UV 220 nm. This resulted in 190-5 (152 mg, 79.12%) as a reddish brown solid.

Step 6: Synthesis of 190-6. To a stirred solution of 190-5 (150 mg, 0.529 mmol, 1 equiv.) and pyridine (213 uL, 2.647 mmol, 5.00 equiv.) in DCM (7.5 mL) was added 6-[4-(trifluoromethyl)pyrazol-1-yl]pyridine-3-sulfonyl chloride (165 mg, 0.529 mmol, 1.00 equiv.) in portions at room temperature under a nitrogen atmosphere. The resulting mixture was stirred for 16 h at room temperature under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 70% to 75% gradient in 25 min; detector: UV 220 nm. This resulted in 190-6 (219 mg, 74.06%) as a light brown solid.

Step 7: Synthesis of N-(2-methyl-3-oxo-2,3-dihydro-1H-indazol-4-yl)-6-(4-(trifluoromethyl)-1H-pyrazol-1-yl)pyridine-3-sulfonamide (1-190). A solution of 190-6 (219 mg, 0.392 mmol, 1 equiv.) and TFA (6 mL, 80.778 mmol, 206.02 equiv.) in DCM (6 mL) was stirred for 15 h at 50° C. under a nitrogen atmosphere. The resulting mixture was concentrated under reduced pressure. The crude product (90 mg) was purified by prep-HPLC with the following conditions: column: XBridge Prep OBD C18, 30*150 mm, 5 m; mobile phase A: water (10 mmol/L NH₄HCO₃)+0.05% NH3. H₂O, mobile phase B: ACN; flow rate 60 mL/min; gradient: 25% B to 48% B in 7 min; wavelength: 254 nm/220 nm; Rt1(min): 8.08) to afford N-(2-methyl-3-oxo-2,3-dihydro-1H-indazol-4-yl)-6-(4-(trifluoromethyl)-1H-pyrazol-1-yl)pyridine-3-sulfonamide (5.9 mg, 3.31%) as a pink solid. LCMS: (ES, m/z): [M+H]⁺439; ¹H NMR (400 MHz, DMSO-d₆): δ 3.32 (s, 3H), 6.92 (d, J8.0 Hz, 1H), 6.96 (d, J8.0 Hz, 1H), 7.39 (t, J8.0 Hz, 1H), 8.11 (d, J 8.8 Hz, 1H), 8.38 (s, 1H), 8.52 (dd, J 8.8, 2.4 Hz, 1H), 8.99 (d, J2.0 Hz, 1H), 9.25 (s, 1H), 10.65 (s, 2H).

### Example 259—Synthesis of 6-(4-(1,1-Difluoroethyl)-1H-Pyrazol-1-yl)-N-(6-Methoxy-1-Methyl-1H-Indazol-7-yl)Pyridine-3-Sulfonamide (I-191)

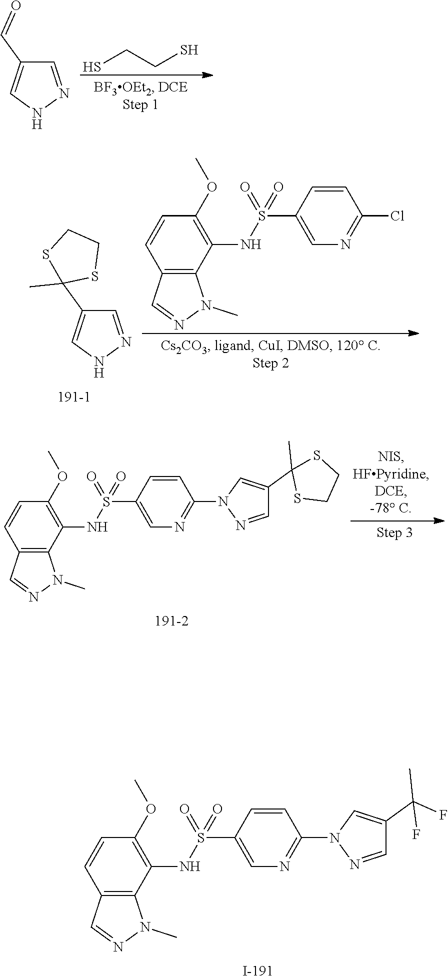

Step 1: Synthesis of 191-1. To a stirred solution of 1-(1H-pyrazol-4-yl) ethanone (500 mg, 4.541 mmol, 1 equiv.) and 1,2-ethanedithiol (513.22 mg, 5.449 mmol, 1.2 equiv.) in dioxane (5 mL) was added BF₃·Et₂O (1933.37 mg, 13.623 mmol, 3 equiv.) dropwise/in portions at 0° C. under a nitrogen atmosphere. The resulting mixture was stirred for 4 h at 80° C. under a nitrogen atmosphere. The reaction was quenched with water (20.00 mL) at room temperature. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 191-1 (800 mg, 94.58%) as an off-white solid.

Step 2: Synthesis of 191-2. A solution of 191-1 (100 mg, 0.537 mmol, 1 equiv.), 6-chloro-N-(6-methoxy-1-methylindazol-7-yl)pyridine-3-sulfonamide (227.25 mg, 0.644 mmol, 1.2 equiv.), Cs₂CO₃ (524.70 mg, 1.611 mmol, 3 equiv.), EPhos (28.71 mg, 0.054 mmol, 0.1 equiv.) and EPhos Pd G4 (28.71 mg, 0.054 mmol, 0.1 equiv.) in dioxane (5 mL, 59.020 mmol, 109.95 equiv.) was stirred overnight at 90° C. under a nitrogen atmosphere. The reaction was quenched with water (20.00 mL) at room temperature. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. This resulted in N-(6-methoxy-1-methylindazol-7-yl)-6-[4-(2-methyl-1,3-dithiolan-2-yl)pyrazol-1-yl]pyridine-3-sulfonamide (60 mg, 22.24%) as an off-white solid.

Step 3: Synthesis of 6-(4-(1,1-difluoroethyl)-1H-pyrazol-1-yl)-N-(6-methoxy-1-methyl-1H-indazol-7-yl)pyridine-3-sulfonamide (I-191). In a 50-mL round bottom flask, to a solution of NIS (134.28 mg, 0.597 mmol, 3 equiv.) in DCE (8 mL) was added dropwise HF-pyridine (98.59 mg, 0.995 mmol, 5 equiv.) at −78 degrees C. under an N₂ atmosphere. The reaction mixture was stirred at −78 degrees C. for 10 mins at −78 degrees. Then a solution of 191-2 (100 mg, 0.199 mmol, 1 equiv.) in DCE (4 mL) was added dropwise and the mixture was stirred for another 30 mins. The reaction was quenched with water/sat. NaHCO₃ (20 mL) and water/sat. Na₂S₂O₃(20 mL), and then the mixture was extracted with DCM (2*20 mL). The combined organic extracts were washed with brine (10 mL), dried over anhydrous Na₂SO₄, and concentrated under vacuum to yield a crude product. The residue was purified by reversed-phase flash chromatography with the following conditions: column: C18 silica gel; mobile phase: MeCN in water (0.1% NH3. H₂O), 40% to 50% gradient in 10 min; detector: UV 254 nm. This resulted in 6-(4-(1,1-difluoroethyl)-1H-pyrazol-1-yl)-N-(6-methoxy-1-methyl-1H-indazol-7-yl)pyridine-3-sulfonamide (12.3 mg, 13.36%) as an off-white solid. LCMS: (ES, m/z): [M+H]⁺ 449; ¹H NMR (400 MHz, DMSO-d₆): δ 2.07 (t, J 18.8 Hz, 1H), 3.18 (s, 3H), 4.27 (s, 3H), 6.84 (d, J 9.2 Hz, 1H), 7.71 (d, J8.8 Hz, 1H), 8.02 (s, 1H), 8.11-8.20 (m, 2H), 8.26 (dd, J8.8, 2.0 Hz, 1H), 8.64 (d, J2.0 Hz, 1H), 9.02 (s, 1H).

### Example 260—Synthesis of N-(1,6-Dimethyl-1H-Pyrazolo[4,3-C]Pyridin-7-yl)-6-(4-(Trifluoromethyl)-1H-Pyrazol-1-yl)Pyridine-3-Sulfonamide (I-189)

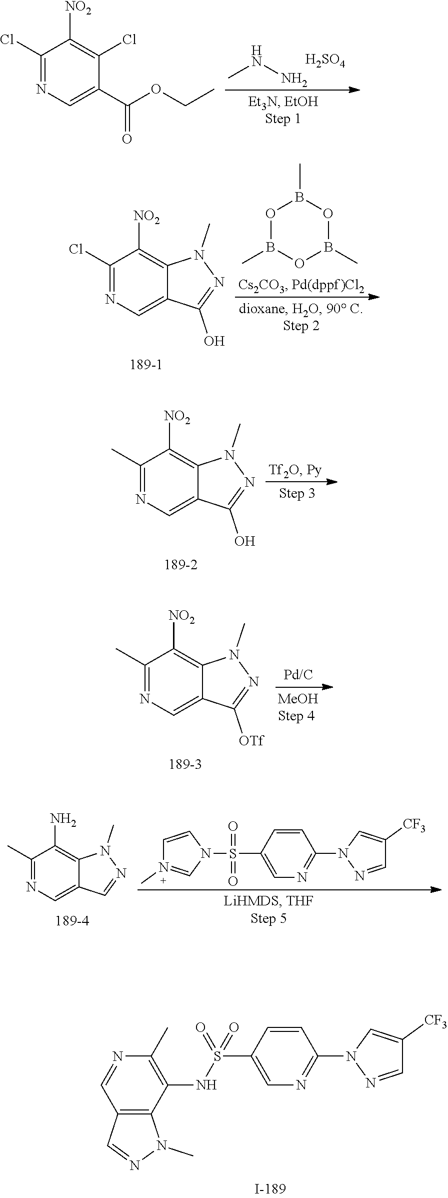

Step 1: Synthesis of 189-1. A solution of ethyl 4,6-dichloro-5-nitropyridine-3-carboxylate (1.056 g, 3.98 mmol, 1 equiv.), Et₃N (1209.51 mg, 11.95 mmol, 3 equiv.) and methyl hydrazine sulfuric acid salt (631.75 mg, 4.38 mmol, 1.1 equiv.) in EtOH (20 mL) was stirred overnight at room temperature. After the reaction was complete, the solution was concentrated under vacuum. The residue was purified by reversed-phase flash chromatography with the following conditions: column: C18 silica gel; mobile phase: ACN in water, 0% to 100% gradient in 20 min; detector: UV 254 nm to give 189-1 (449 mg, 49.30%) as a red solid.

Step 2: Synthesis of 189-2. A solution of 189-1 (930 mg, 4.068 mmol, 1 equiv.), trimethyl-1,3,5,2,4,6-trioxatriborinane (766.05 mg, 6.102 mmol, 1.5 equiv.), Pd(dppf)Cl₂ (595.38 mg, 0.813 mmol, 0.2 equiv.) and Cs₂CO₃ (2.65 g, 8.14 mmol, 2 equiv.) in dioxane (45 mL) was stirred overnight at 90° C. under a nitrogen atmosphere. After the reaction was complete, the resulting mixture was diluted with water (100 mL). The resulting mixture was extracted with EtOAc (4×100 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water (0.1% NH₃. H₂O), 0% to 100% gradient in 35 min; detector: UV 254 nm to give 189-2 (780 mg, 92.10%) as a yellow solid.

Step 3: Synthesis of 189-3. A solution of 189-2 (423 mg, 4.07 mmol, 1 equiv.), pyridine (1.284 g, 15.444 mmol, 4 equiv.) and Tf₂O (2.864 g, 10.16 mmol, 2.5 equiv.) in DCM (40 mL) was stirred overnight at room temperature under a nitrogen atmosphere. After the reaction was complete, the resulting mixture was diluted with water (50 mL). The resulting mixture was extracted with EtOAc (4×60 mL). The combined organic layers were washed with brine (100 mL) and dried over anhydrous Na₂SO₄. After filtration, the filtrate was concentrated under reduced pressure. The residue was by prep-TLC (PE/EA 5:1) to afford 189-3 (465 mg, 33.63%) as a light-yellow solid.

Step 4: Synthesis of 189-4. A mixture of 189-3 (465 mg, 1.368 mmol, 1 equiv.) and Pd/C (150 mg, 1.41 mmol, 1.03 equiv.) in MeOH (60 mL) was stirred overnight at room temperature under a hydrogen atmosphere. After the reaction was complete, the solid was filtered. The filtrate was concentrated under vacuum to give 189-4 (432 mg, 97.44%) as a light-yellow solid.

Step 5: Synthesis of N-(1,6-dimethyl-1H-pyrazolo[4,3-c]pyridin-7-yl)-6-(4-(trifluoromethyl)-1H-pyrazol-1-yl)pyridine-3-sulfonamide (1-189). A solution of 189-4 (162 mg, 0.999 mmol, 1 equiv.) in THE (10 mL) was treated with LiHMDS (417.82 mg, 2.498 mmol, 2.5 equiv.) for 10 min at −30° C. followed by the addition of 3-methyl-1-{6-[4-(trifluoromethyl) pyrazol-1-yl] pyridin-3-ylsulfonyl} imidazol-1-ium (429.46 mg, 1.199 mmol, 1.20 equiv.) in portions at −10° C. Then the reaction was stirred from −10° C. to room temperature for 3 h. After the reaction was complete, the resulting mixture was quenched with water (50 mL). The resulting mixture was extracted with EtOAc (4×60 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: ACN in water, 0% to 100% gradient in 30 min; detector: UV 254 nm to give the crude. The crude was purified again by prep-HPLC with the following conditions: column: Xselect CSH C₁₈ OBD, 30*150 mm 5 m; mobile phase A: water (0.1% FA), mobile phase B: ACN; flow rate 60 mL/min; gradient: 23% B to 34% B in 7 min; wavelength: 254 nm/220 nm; Rt1(min): 6.3) to afford N-(1,6-dimethyl-1H-pyrazolo[4,3-c]pyridin-7-yl)-6-(4-(trifluoromethyl)-1H-pyrazol-1-yl)pyridine-3-sulfonamide (31.9 mg, 7.28%) as a white solid. LCMS: (ES, m/z): [M+H]⁺ 438; ¹H NMR (400 MHz, DMSO-d₆) δ 1.86 (s, 3H), 4.25 (s, 3H), 8.17-8.28 (m, 3H), 8.42 (s, 1H), 8.67 (d, J 1.6 Hz, 1H), 8.87 (s, 1H), 9.32 (s, 1H), 10.63 (s, 1H).

### Example 261—Synthesis of N-(2-Methyl-3-Oxo-2,3-Dihydro-1H-Indazol-4-yl)-6-(4-(Trifluoromethyl)-1H-Pyrazol-1-yl)Pyridine-3-Sulfonamide (I-190)

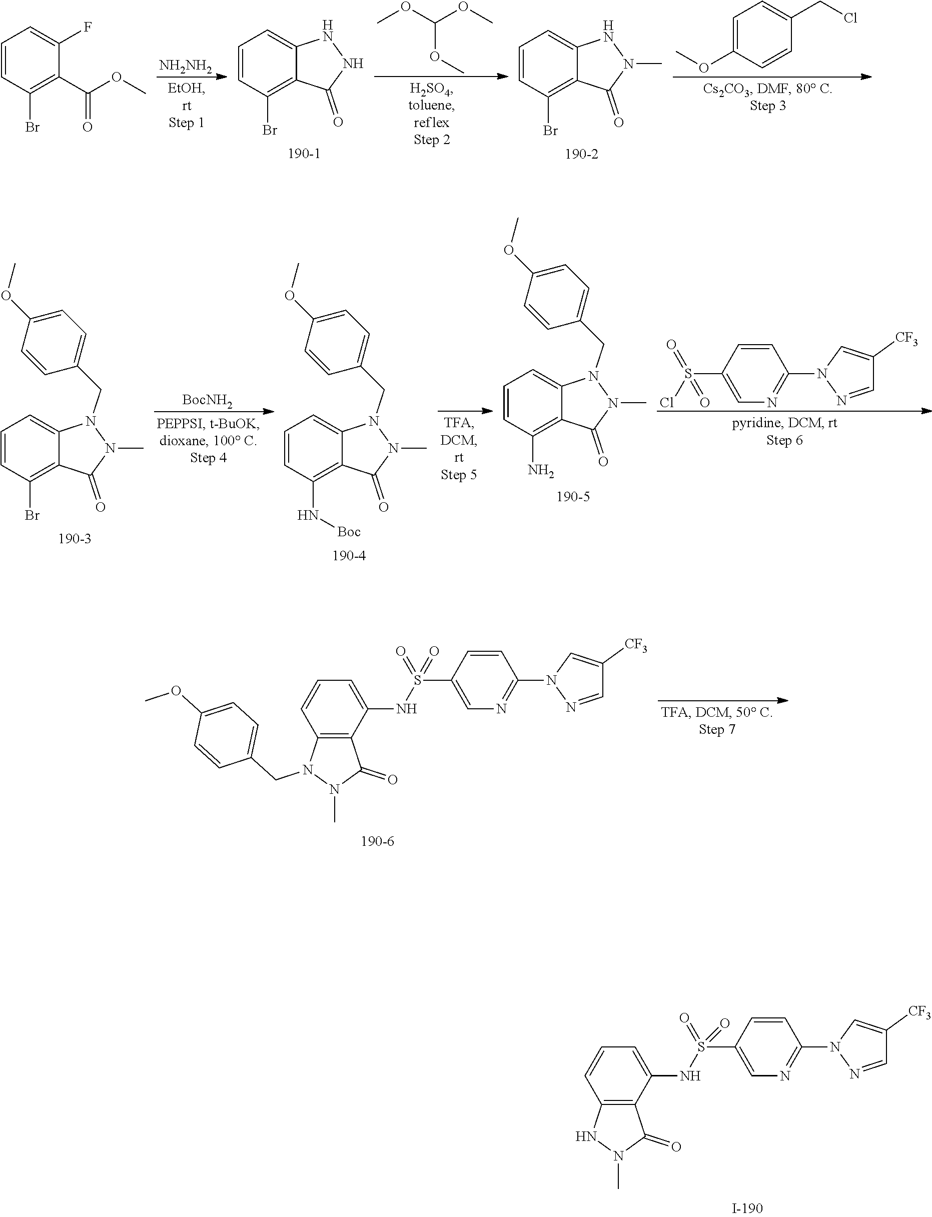

Step 1: Synthesis of 190-1. To a stirred solution of methyl 2-bromo-6-fluorobenzoate (5 g, 21.456 mmol, 1 equiv.) in EtOH (50 mL) was added hydrazine hydrate (10.74 g, 214.560 mmol, 10 equiv.) dropwise at room temperature under a nitrogen atmosphere. The resulting mixture was stirred for 16 h at 80° C. under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 10% to 12% gradient in 15 min; detector: UV 220 nm. This resulted in 190-1 (2.63 g, 57.54%) as a brown solid.

Step 2: Synthesis of 190-2. To a stirred solution of 190-1 (1.42 g, 6.666 mmol, 1 equiv.) and trimethyl orthoformate (2.48 g, 23.331 mmol, 3.5 equiv.) in toluene (14 mL) was added H₂SO₄ (391 uL, 7.336 mmol, 1.10 equiv.) dropwise at room temperature under a nitrogen atmosphere. The resulting mixture was stirred for 4 h at 110° C. under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 10% to 20% gradient in 10 min; detector: UV 220 nm. This resulted in 190-2 (870 mg, 57.48%) as a brown solid.

Step 3: Synthesis of 190-3. To a stirred solution of 190-2 (740 mg, 3.259 mmol, 1 equiv.) and Cs₂CO₃ (3.19 g, 9.777 mmol, 3.00 equiv.) in DMF (18 mL) was added 4-methoxybenzyl chloride (920 uL, 6.515 mmol, 2.00 equiv.) dropwise at room temperature under a nitrogen atmosphere. The resulting mixture was stirred for 3 h at 80° C. under a nitrogen atmosphere. The precipitated solids were collected by filtration and washed with MeCN (3×18 mL). The filtrate was concentrated under reduced pressure. The residue was purified by reversed-phase flash chromatography with the following conditions: column: C18 silica gel; mobile phase: MeCN in water (10 mmol/L NH₄HCO₃), 45% to 47% gradient in 30 min; detector: UV 210 nm. This resulted in 190-3 (776 mg, 68.58%) as a white solid.

Step 4: Synthesis of 190-4. To a stirred solution of 190-3 (490 mg, 1.411 mmol, 1 equiv.), t-BuOK (475.1 mg, 4.233 mmol, 3 equiv.) and {1,3-bis[2,6-bis(pentan-3-yl)phenyl]-4,5-dichloro-2,3-dihydro-1H-imidazol-2-yl}dichloro(2-methyl-1lambda4-pyridin-1-yl)palladium (237.4 mg, 0.282 mmol, 0.2 equiv.) in dioxane (24 mL) was added tert-butyl carbamate (245 mg, 2.117 mmol, 1.5 equiv.) in portions at room temperature under a nitrogen atmosphere. The resulting mixture was stirred for 3 h at 100° C. under a nitrogen atmosphere. The resulting mixture was filtered and the filter cake was washed with MeCN (3×8 mL). The filtrate was concentrated under reduced pressure. The residue was purified by reversed-phase flash chromatography with the following conditions: column: C18 silica gel; mobile phase: MeCN in water (10 mmol/L NH₄HCO₃), 60% to 65% gradient in 10 min; detector: UV 220 nm. This resulted in 190-4 (260 mg, 48.05%) as a brown solid.

Step 5: Synthesis of 190-5. To a stirred solution of 190-4 (260 mg, 0.678 mmol, 1 equiv.) in DCM (13 mL) was added TFA (2.6 mL, 35.004 mmol, 51.62 equiv.) dropwise at room temperature under a nitrogen atmosphere. The resulting mixture was stirred for 1 h at room temperature under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 48% to 50% gradient in 20 min; detector: UV 220 nm. This resulted in 190-5 (152 mg, 79.12%) as a reddish brown solid.

Step 6: Synthesis of 190-6. To a stirred solution of 190-5 (150 mg, 0.529 mmol, 1 equiv.) and pyridine (213 uL, 2.647 mmol, 5.00 equiv.) in DCM (7.5 mL) was added 6-[4-(trifluoromethyl)pyrazol-1-yl]pyridine-3-sulfonyl chloride (165 mg, 0.529 mmol, 1.00 equiv.) in portions at room temperature under a nitrogen atmosphere. The resulting mixture was stirred for 16 h at room temperature under a nitrogen atmosphere. The resulting mixture was concentrated under reduced pressure. The residue was purified by reversed-phase flash chromatography with the following conditions: column: C18 silica gel; mobile phase: MeCN in water (10 mmol/L NH₄HCO₃), 70% to 75% gradient in 25 min; detector: UV 220 nm. This resulted in 190-6 (219 mg, 74.06%) as a light brown solid.

Step 7: Synthesis of N-(2-methyl-3-oxo-2,3-dihydro-1H-indazol-4-yl)-6-(4-(trifluoromethyl)-1H-pyrazol-1-yl)pyridine-3-sulfonamide (1-190). A solution of 190-6 (219 mg, 0.392 mmol, 1 equiv.) and TFA (6 mL, 80.778 mmol, 206.02 equiv.) in DCM (6 mL) was stirred for 15 h at 50° C. under a nitrogen atmosphere. The resulting mixture was concentrated under reduced pressure. The crude product (90 mg) was purified by prep-HPLC with the following conditions: column: XBridge Prep OBD C18, 30*150 mm, 5 m; mobile phase A: water (10 mmol/L NH₄HCO₃)+0.05% NH3. H₂O, mobile phase B: ACN; flow rate 60 mL/min; gradient: 25% B to 48% B in 7 min; wavelength: 254 nm/220 nm; Rt1(min): 8.08) to afford N-(2-methyl-3-oxo-2,3-dihydro-1H-indazol-4-yl)-6-(4-(trifluoromethyl)-1H-pyrazol-1-yl)pyridine-3-sulfonamide (5.9 mg, 3.31%) as a pink solid. LCMS: (ES, m/z): [M+H]⁺ 439; ¹H NMR (400 MHz, DMSO-d₆): δ 3.32 (s, 3H), 6.92 (d, J8.0 Hz, 1H), 6.96 (d, J8.0 Hz, 1H), 7.39 (t, J8.0 Hz, 1H), 8.11 (d, J 8.8 Hz, 1H), 8.38 (s, 1H), 8.52 (dd, J 8.8, 2.4 Hz, 1H), 8.99 (d, J2.0 Hz, 1H), 9.25 (s, 1H), 10.65 (s, 2H).

### Example 262—Synthesis of 6-(4-(1,1-Difluoroethyl)-1H-Pyrazol-1-yl)-N-(6-Methoxy-1-Methyl-1H-Indazol-7-yl)Pyridine-3-Sulfonamide (I-191)

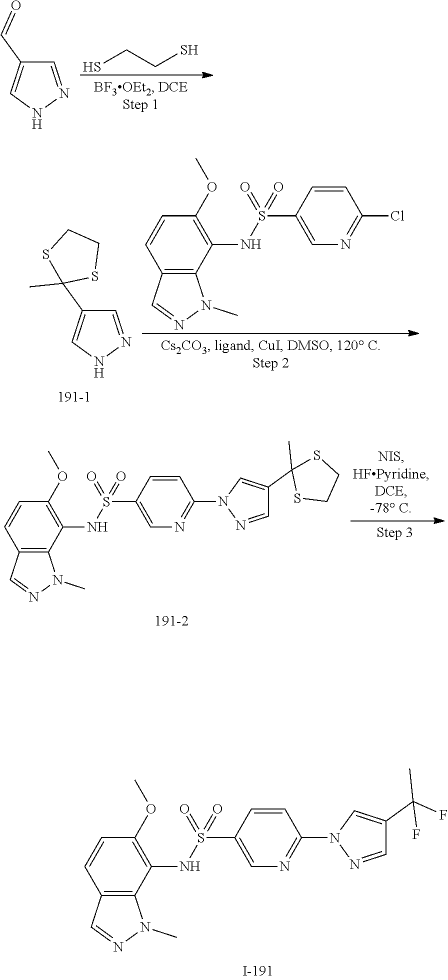

Step 1: Synthesis of 191-1. To a stirred solution of 1-(1H-pyrazol-4-yl) ethanone (500 mg, 4.541 mmol, 1 equiv.) and 1,2-ethanedithiol (513.22 mg, 5.449 mmol, 1.2 equiv.) in dioxane (5 mL) was added BF₃·Et₂O (1933.37 mg, 13.623 mmol, 3 equiv.) dropwise/in portions at 0° C. under a nitrogen atmosphere. The resulting mixture was stirred for 4 h at 80° C. under a nitrogen atmosphere. The reaction was quenched with water (20.00 mL) at room temperature. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 191-1 (800 mg, 94.58%) as an off-white solid.

Step 2: Synthesis of 191-2. A solution of 191-1 (100 mg, 0.537 mmol, 1 equiv.), 6-chloro-N-(6-methoxy-1-methylindazol-7-yl)pyridine-3-sulfonamide (227.25 mg, 0.644 mmol, 1.2 equiv.), Cs₂CO₃ (524.70 mg, 1.611 mmol, 3 equiv.), EPhos (28.71 mg, 0.054 mmol, 0.1 equiv.) and EPhos Pd G4 (28.71 mg, 0.054 mmol, 0.1 equiv.) in dioxane (5 mL, 59.020 mmol, 109.95 equiv.) was stirred overnight at 90° C. under a nitrogen atmosphere. The reaction was quenched with water (20.00 mL) at room temperature. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. This resulted in N-(6-methoxy-1-methylindazol-7-yl)-6-[4-(2-methyl-1,3-dithiolan-2-yl)pyrazol-1-yl]pyridine-3-sulfonamide (60 mg, 22.24%) as an off-white solid.

Step 3: Synthesis of 6-(4-(1,1-difluoroethyl)-1H-pyrazol-1-yl)-N-(6-methoxy-1-methyl-1H-indazol-7-yl)pyridine-3-sulfonamide (I-191). In a 50-mL round bottom flask, to a solution of NIS (134.28 mg, 0.597 mmol, 3 equiv.) in DCE (8 mL) was added dropwise HF-pyridine (98.59 mg, 0.995 mmol, 5 equiv.) at −78° C. under an N₂ atmosphere. The reaction mixture was stirred at −78 degrees C. for 10 mins at −78 degrees. Then a solution of 191-2 (100 mg, 0.199 mmol, 1 equiv.) in DCE (4 mL) was added dropwise and the mixture was stirred for another 30 mins. The reaction was quenched with water/sat. NaHCO₃ (20 mL) and water/sat. Na₂S₂O₃ (20 mL), and then the mixture was extracted with DCM (2*20 mL). The combined organic extracts were washed with brine (10 mL), dried over anhydrous Na₂SO₄, and concentrated under vacuum to yield a crude product. The residue was purified by reversed-phase flash chromatography with the following conditions: column: C18 silica gel; mobile phase: MeCN in water (0.1% NH₃·H₂O), 40% to 50% gradient in 10 min; detector: UV 254 nm. This resulted in 6-(4-(1,1-difluoroethyl)-1H-pyrazol-1-yl)-N-(6-methoxy-1-methyl-1H-indazol-7-yl)pyridine-3-sulfonamide (12.3 mg, 13.36%) as an off-white solid. LCMS: (ES, m/z): [M+H]⁺ 449; ¹H NMR (400 MHz, DMSO-d₆): δ 2.07 (t, J 18.8 Hz, 1H), 3.18 (s, 3H), 4.27 (s, 3H), 6.84 (d, J 9.2 Hz, 1H), 7.71 (d, J8.8 Hz, 1H), 8.02 (s, 1H), 8.11-8.20 (m, 2H), 8.26 (dd, J8.8, 2.0 Hz, 1H), 8.64 (d, J2.0 Hz, 1H), 9.02 (s, 1H).

### Example 263—Synthesis of 6-(4-(Difluoromethyl)-1H-Pyrazol-1-yl)-N-(6-Methoxy-1-Methyl-1H-Pyrazolo[4,3-C]Pyridin-7-yl)Pyridine-3-Sulfonamide (I-192)

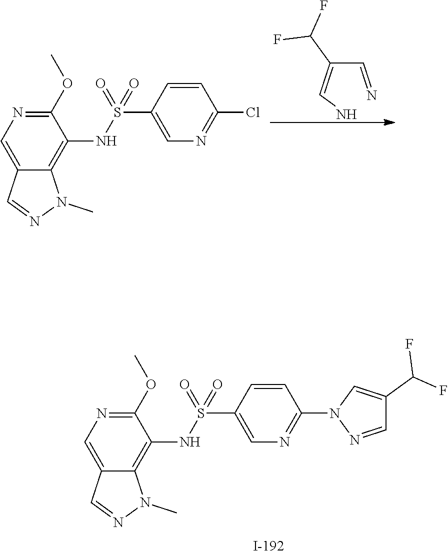 [SMILES: COc1ncc2cnn(C)c2c1NS(=O)(=O)c1ccc(Cl)nc1 ; N=C/C(=C\N)C(F)F ; COc1ncc2cnn(C)c2c1NS(=O)(=O)c1ccc(-n2cc(C(F)F)cn2)nc1]

A solution of 6-chloro-N-(6-methoxy-1-methyl-1H-pyrazolo[4,3-c]pyridin-7-yl)pyridine-3-sulfonamide (80 mg, 0.226 mmol, 1.00 equiv.), 4-(difluoromethyl)-1H-pyrazole (32.0 mg, 0.271 mmol, 1.20 equiv.), t-BuXPhos (19.2 mg, 0.045 mmol, 0.20 equiv.), tBuXPhos Pd G3 (17.9 mg, 0.023 mmol, 0.10 equiv.) and Cs₂CO₃ (147.3 mg, 0.452 mmol, 2.00 equiv.) in dioxane (4 mL) was stirred for 2 h at 100° C. under a nitrogen atmosphere. The mixture was allowed to cool down to room temperature. The resulting mixture was diluted with water (20 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃⁺0.05% NH₃·H₂O), mobile phase B: ACN; flow rate 60 mL/min; gradient: 12% B to 40% B in 7 min; wavelength: 254 nm/220 nm; Rt1(min): 8.95 to afford 6-(4-(difluoromethyl)-1H-pyrazol-1-yl)-N-(6-methoxy-1-methyl-1H-pyrazolo[4,3-c]pyridin-7-yl)pyridine-3-sulfonamide (20 mg, 20.09%) as a white solid. LCMS: (ES, m/z): [M+H]⁺ 436; ¹H NMR (DMSO-d₆, 400 MHz): δ 3.28 (s, 3H), 4.25 (s, 3H), 7.16 (t, J 55.2 Hz, 1H), 8.14 (d, J 8.8 Hz, 1H), 8.20(d, J 8.8 Hz, 1H), 8.22 (s, 1H), 8.25 (dd, J 8.4 Hz, 2.4 Hz, 1H), 8.67 (s, 1H), 8.38 (s, 1H), 9.03 (d, J 2.0 Hz, 1H), 10.23 (s, 1H).

### Example 264—Synthesis of 6-(4-Fluoro-1H-Pyrazol-1-yl)-N-(6-Methoxy-1-Methyl-1H-Pyrazolo[4,3-C]Pyridin-7-yl)Pyridine-3-Sulfonamide (I-193)

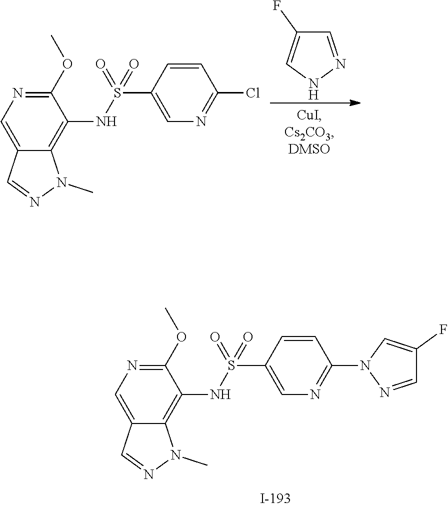

A solution of 6-chloro-N-(6-methoxy-1-methyl-1H-pyrazolo[4,3-c]pyridin-7-yl)pyridine-3-sulfonamide (80 mg, 0.226 mmol, 1.00 equiv.), (1S,2S)-1,2-diethylcyclohexane (16.08 mg, 0.113 mmol, 0.50 equiv.), 4-fluoro-1H-pyrazole (25.30 mg, 0.294 mmol, 1.30 equiv.), Cs₂CO₃ (221.03 mg, 0.678 mmol, 3 equiv.) and CuI (21.53 mg, 0.113 mmol, 0.50 equiv.) in DMSO (5 mL) was stirred for 2 h at 140° C. under a nitrogen atmosphere. The mixture was allowed to cool down to room temperature. The resulting mixture was diluted with water (20 mL). The resulting mixture was extracted with EtOAc (3×10 mL). The combined organic layers were washed with brine (50 mL) and dried over anhydrous Na₂SO₄. After filtration, the filtrate was concentrated under reduced pressure. The crude product was purified by prep-HPLC with the following conditions: column: XBridge Shield RP18 OBD, 30*150 mm, 5 m; mobile phase A: water (10 mmol/L NH₄HCO₃+0.05% NH₃H₂O), mobile phase B: ACN; flow rate 60 mL/min; gradient: 12% B to 42% B in 7 min; wavelength: 254 nm/220 nm; Rt1(min): 6 to afford 6-(4-fluoro-1H-pyrazol-1-yl)-N-(6-methoxy-1-methyl-1H-pyrazolo[4,3-c]pyridin-7-yl)pyridine-3-sulfonamide (11.2 mg, 11.92%) as an off-white solid. LCMS: (ES, m/z): [M+H]⁺ 404; ¹H NMR (400 MHz, DMSO-d₆) δ 3.27 (s, 3H), 4.25 (s, 3H), 8.07(d, J 2.8 Hz, 1H), 8.09 (d, J 8.4 Hz, 1H), 8.21(d, J 2.8 Hz, 1H), 8.22 (d, J 2.4 Hz, 1H), 8.61 (s, 1H), 8.62 (s, 1H), 8.82 (d, J 4.6 Hz, 1H).

### Example 265—Synthesis of 6-(4-Isopropyl-1H-Pyrazol-1-yl)-N-(6-Methoxy-1-Methyl-1H-Pyrazolo[4,3-C]Pyridin-7-yl)Pyridine-3-Sulfonamide (I-194)

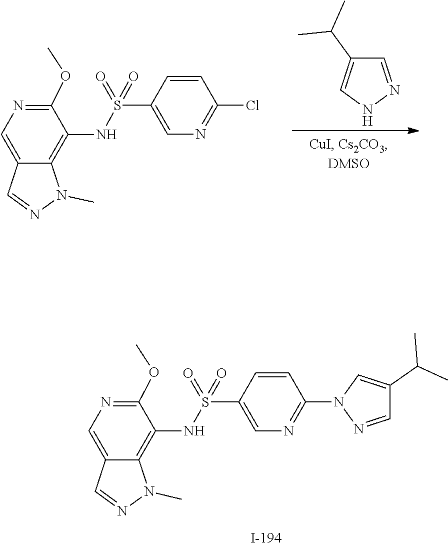

A solution of 6-chloro-N-(6-methoxy-1-methyl-1H-pyrazolo[4,3-c]pyridin-7-yl)pyridine-3-sulfonamide (90 mg, 0.254 mmol, 1.00 equiv.), 4-isopropyl-1H-pyrazole (36.43 mg, 0.330 mmol, 1.3 equiv.), (1S,2S)-1,2-diethylcyclohexane (10.86 mg, 0.076 mmol, 0.30 equiv.), CuI (14.53 mg, 0.076 mmol, 0.30 equiv.) and Cs₂CO₃ (248.66 mg, 0.762 mmol, 3.00 equiv.) in DMSO (2 mL) was stirred for 2 h at 140° C. under a nitrogen atmosphere. The mixture was allowed to cool down to room temperature. The resulting mixture was diluted with water (20 mL). The resulting mixture was extracted with EtOAc (3×10 mL). The combined organic layers were washed with brine (30 mL) and dried over anhydrous Na₂SO₄. After filtration, the filtrate was concentrated under reduced pressure. The crude product was purified by prep-HPLC with the following conditions: column: column: XBridge Prep OBD C18, 30*150 mm, 5 m; mobile phase A: water (10 mmol/L NH₄HCO₃+0.05% NH₃·H₂O), mobile phase B: ACN; flow rate 60 mL/min; gradient: 27% B to 54% B in 7 min; wavelength: 254 nm/220 nm; Rt1(min): 8.53 to afford 6-(4-isopropyl-1H-pyrazol-1-yl)-N-(6-methoxy-1-methyl-1H-pyrazolo[4,3-c]pyridin-7-yl)pyridine-3-sulfonamide (49.2 mg, 43.84%) as an off-white solid. LCMS: (ES, m/z): [M+H]⁺ 428; ¹H NMR (400 MHz, DMSO-d₆) δ 1.24 (d, J 6.8 Hz, 6H), 2.86-2.94 (m, 1H), 3.29 (s, 3H), 4.24 (s, 3H), 7.86 (d, J 0.8 Hz, 1H), 8.06 (d, J 8.8 Hz, 1H), 8.18 (dd, J 8.8, 2.4 Hz, 1H), 8.23 (s, 1H), 8.46 (d, J 0.8 Hz, 1H), 8.56 (d, J 2.0 Hz, 1H), 8.65 (s, 1H), 10.14 (s, 1H).

### Example 266—Synthesis of N-(6-Methoxy-1-Methyl-1H-Pyrazolo[4,3-C]Pyridin-7-yl)-N-Methyl-6-(3-(Trifluoromethyl)-1H-Pyrazol-1-yl)Pyridine-3-Sulfonamide (I-195)

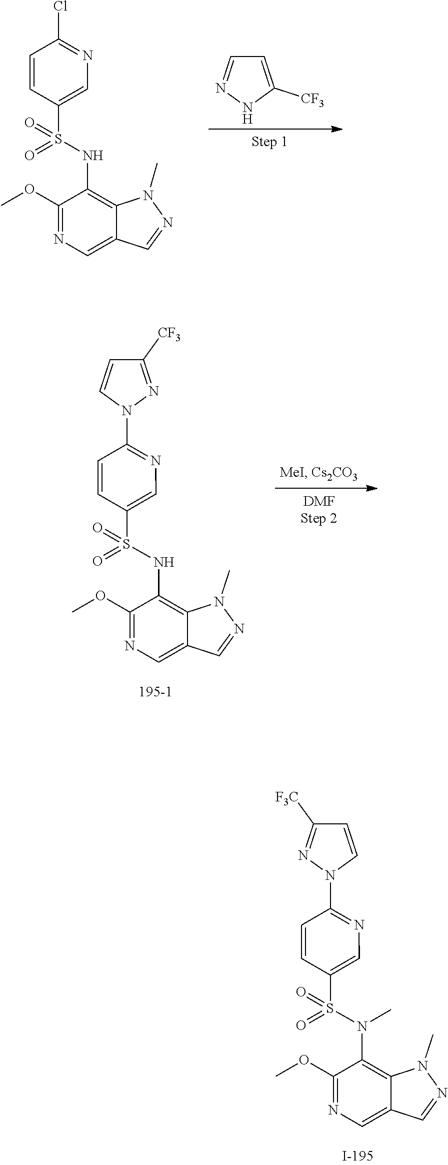 [SMILES: COc1ncc2cnn(C)c2c1NS(=O)(=O)c1ccc(Cl)nc1 ; FC(F)(F)c1ccn[nH]1 ; COc1ncc2cnn(C)c2c1NS(=O)(=O)c1ccc(-n2ccc(C(F)(F)F)n2)nc1 ; COc1ncc2cnn(C)c2c1N(C)S(=O)(=O)c1ccc(-n2ccc(C(F)(F)F)n2)nc1]

Step 1: Synthesis of 195-1. A mixture of 6-chloro-N-(6-methoxy-1-methyl-1H-pyrazolo[4,3-c]pyridin-7-yl)pyridine-3-sulfonamide (200.00 mg, 0.565 mmol, 1 equiv.), CuI (53.83 mg, 0.282 mmol, 0.5 equiv.), 5-(trifluoromethyl)-1H-pyrazole (40.21 mg, 0.282 mmol, 0.5 equiv.) and Cs₂CO₃ (552.58 mg, 1.695 mmol, 3 equiv.) in DMSO (3 mL) was stirred for 16 h at 140° C. under a nitrogen atmosphere. The resulting mixture was diluted with water (10 mL). The resulting mixture was extracted with EtOAc (3×15 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 10% to 50% gradient in 10 min; detector: UV 254 nm, to afford N-(6-methoxy-1-methyl-1H-pyrazolo[4,3-c]pyridin-7-yl)-6-(3-(trifluoromethyl)-1H-pyrazol-1-yl)pyridine-3-sulfonamide (130.00 mg, 50.26%) as an off-white solid.

Step 2: Synthesis of N-(6-methoxy-1-methyl-11H-pyrazolo[4,3-c]pyridin-7-yl)-N-methyl-6-(3-(trifluoromethyl)-11H-pyrazol-1-yl)pyridine-3-sulfonamide (1-195). A solution of N-(6-methoxy-1-methyl-1H-pyrazolo[4,3-c]pyridin-7-yl)-6-(3-(trifluoromethyl)-1H-pyrazol-1-yl)pyridine-3-sulfonamide (100.00 mg, 0.221 mmol, 1 equiv.) in DMF (3 mL) was treated with Cs₂CO₃ (216.25 mg, 0.663 mmol, 3 equiv.) for 30 min at room temperature under a nitrogen atmosphere followed by the addition of methyl iodide (62.61 mg, 0.442 mmol, 2 equiv.) dropwise at 0° C. The resulting mixture was stirred for 30 min at room temperature. The resulting mixture was diluted with water (10 mL). The resulting mixture was extracted with EtOAc (3×15 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Prep OBD C18, 30*150 mm, 5 m; mobile phase A: water (10 mmol/L NH₄HCO₃+0.05% NH₃H₂O), mobile phase B: ACN; flow rate 60 mL/min; gradient: 45% B to 70% B in 7 min; wavelength: 220 nm; Rt1(min): 7.58, to afford N-(6-methoxy-1-methyl-1H-pyrazolo[4,3-c]pyridin-7-yl)-N-methyl-6-(3-(trifluoro-methyl)-1H-pyrazol-1-yl)pyridine-3-sulfonamide (24.10 mg, 23.14%) as an off-white solid. LCMS: (ES, m/z): [M+H]⁺ 468; ¹H NMR (400 MHz, DMSO-d₆) δ 3.33 (s, 3H), 3.37 (s, 3H), 4.22 (s, 3H), 7.18 (d, J 2.8 Hz, 1H), 8.21 (d, J 8.8 Hz, 1H), 8.27 (s, 1H), 8.35 (d, J 2.4 Hz, 1H), 8.75 (s, 1H), 8.80 (d, J 2.4 Hz, 1H), 8.96 (d, J 2.4 Hz, 1H).

### Example 267—Synthesis of 6-(4-(Difluoromethyl)-1H-Pyrazol-1-yl)-N-(6-Methoxy-1-Methyl-1H-Pyrazolo[4,3-C]Pyridin-7-yl)Pyridine-3-Sulfonamide (I-196)

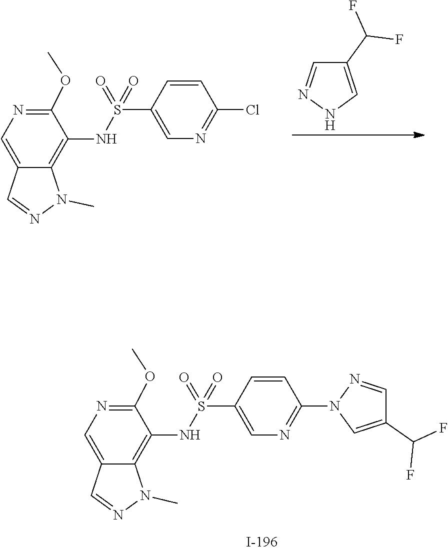 [SMILES: COc1ncc2cnn(C)c2c1NS(=O)(=O)c1ccc(Cl)nc1 ; FC(F)c1cn[nH]c1 ; COc1ncc2cnn(C)c2c1NS(=O)(=O)c1ccc(-n2cc(C(F)F)cn2)nc1]

A mixture of 6-chloro-N-(6-methoxy-1-methyl-1H-pyrazolo[4,3-c]pyridin-7-yl)pyridine-3-sulfonamide (150.00 mg, 0.424 mmol, 1 equiv.), 4-(difluoromethyl)-1H-pyrazole (60.08 mg, 0.509 mmol, 1.2 equiv.), t-BuXPhos (18.00 mg, 0.042 mmol, 0.1 equiv.), tBuXPhos Pd G3 (67.36 mg, 0.085 mmol, 0.2 equiv.) and Cs₂CO₃ (414.43 mg, 1.272 mmol, 3 equiv.) in 1,4-dioxane (4 mL) was stirred for 4 h at 100° C. under a nitrogen atmosphere. The resulting mixture was diluted with water (10 mL). The resulting mixture was extracted with EtOAc (3×15 mL). The combined organic layers were washed with brine (20 mL) and dried over anhydrous Na₂SO₄. After filtration, the filtrate was concentrated under reduced pressure. The crude product was purified by prep-HPLC with the following conditions: column: XBridge Shield RP18 OBD, 30*150 mm, 5 m; mobile phase A: water (10 mmol/L NH₄HCO₃+0.05% NH₃H₂O), mobile phase B: ACN; flow rate 60 mL/min; gradient: 22% B to 45% B in 7 min; wavelength: 254 nm/220 nm; Rt1(min): 7, to afford 6-(4-(difluoromethyl)-1H-pyrazol-1-yl)-N-(6-methoxy-1-methyl-1H-pyrazolo[4,3-c]pyridin-7-yl)pyridine-3-sulfonamide (50.40 mg, 27.22%) as an off-white solid. LCMS: (ES, m/z): [M+H]⁺ 436; ¹H NMR (400 MHz, DMSO-d₆) δ 3.28 (s, 3H), 4.25 (s, 3H), 6.92 (d, J=2.4 Hz, 1H), 7.20 (t, J=54.0 Hz, 1H), 8.13 (d, J 8.8 Hz, 1H), 8.23 (s, 1H), 8.22 (dd, J 8.8 Hz, 2.4 Hz, 1H), 8.65 (s, 1H), 8.68 (d, J 2.8 Hz, 1H), 8.83 (d, J 2.8 Hz, 1H), 10.32 (s, 1H).

### Example 268—Synthesis of N-(6-Methoxy-1-Methyl-1H-Pyrazolo[4,3-C]Pyridin-7-yl)-6-(3-Methyl-1H-Pyrazol-1-yl)Pyridine-3-Sulfonamide (I-197)

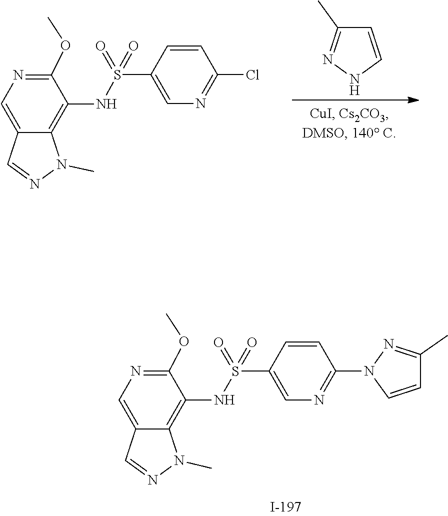

To a stirred solution of 6-chloro-N-{6-methoxy-1-methylpyrazolo[4,3-c]pyridin-7-yl}pyridine-3-sulfonamide (80 mg, 0.226 mmol, 1 equiv.) and 1,2-cyclohexanediamine (38.73 mg, 0.339 mmol, 1.5 equiv.) in DMSO (3 mL) were added 3-methyl-1H-pyrazole (27.85 mg, 0.339 mmol, 1.5 equiv.), Cs₂CO₃ (34.43 mg, 0.452 mmol, 2 equiv.) and CuI (21.53 mg, 0.113 mmol, 0.5 equiv.) in portions at room temperature under a nitrogen atmosphere. The resulting mixture was stirred for 3 h at 140° C. under a nitrogen atmosphere. The reaction was quenched with water (10 mL). The resulting mixture was extracted with EtOAc (2×10 mL). The combined organic layers were washed with brine (2×10 mL) and dried over anhydrous Na₂SO₄. After filtration, the filtrate was concentrated under reduced pressure. The crude product (100 mg) was purified by prep-HPLC with the following conditions: column: YMC-Actus Triart C18 ExRS, 30*150 mm, 5 m; mobile phase A: water(10 mmol/L NH₄HCO₃+0.05% NH₃H₂O), mobile phase B: ACN; flow rate 60 mL/min; gradient: 11% B to 41% B in 7 min; wavelength: 220 nm; Rt1(min): 7.32) to afford N-{6-methoxy-1-methylpyrazolo[4,3-c]pyridin-7-yl}-6-(3-methylpyrazol-1-yl)pyridine-3-sulfonamide (11.7 mg, 12.95%) as a white solid. LCMS: (ES, m/z): [M+H]⁺ 400; ¹H NMR (400 MHz, DMSO-d₆) δ 2.32 (s, 3H), 3.28 (s, 3H), 4.24 (s, 3H), 6.47 (d, J2.8 Hz, 1H), 8.02 (d, J 8.8 Hz, 1H), 8.17 (dd, J8.8, 2.4 Hz, 1H), 8.23 (s, 1H), 8.57-8.58 (m, 2H), 8.64 (s, 1H), 10.12 (s, 1H). ¹H NMR (400 MHz, DMSO-d₆) δ 2.32 (s, 3H), 3.28 (s, 3H), 4.24 (s, 3H), 6.47 (d, J2.8 Hz, 1H), 8.02 (d, J 8.8 Hz, 1H), 8.17 (dd, J8.8, 2.4 Hz, 1H), 8.23 (s, 1H), 8.57-8.58 (m, 2H), 8.64 (s, 1H), 10.12 (s, 1H).

### Example 269—Synthesis of N-(1-Methyl-3-Oxo-2,3-Dihydro-1H-Indazol-4-yl)-6-(4-(Trifluoromethyl)-1H-Pyrazol-1-yl)Pyridine-3-Sulfonamide (I-198)

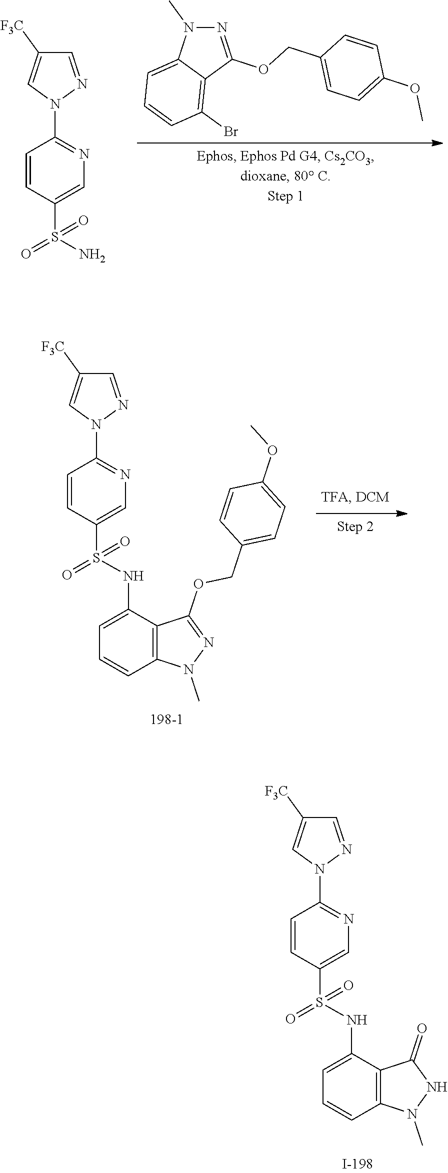 [SMILES: NS(=O)(=O)c1ccc(-n2cc(C(F)(F)F)cn2)nc1 ; COc1ccc(COc2nn(C)c3cccc(Br)c23)cc1 ; COc1ccc(COc2nn(C)c3cccc(NS(=O)(=O)c4ccc(-n5cc(C(F)(F)F)cn5)nc4)c23)cc1 ; Cn1[nH]c(=O)c2c(NS(=O)(=O)c3ccc(-n4cc(C(F)(F)F)cn4)nc3)cccc21]

Step 1: Synthesis of 198-1. A solution of 6-[4-(trifluoromethyl) pyrazol-1-yl]pyridine-3-sulfonamide (200 mg, 0.684 mmol, 1 equiv.), 4-bromo-3-[(4-methoxyphenyl)methoxy]-1-methylindazole (285.15 mg, 0.821 mmol, 1.2 equiv.), Cs₂CO₃ (668.94 mg, 2.052 mmol, 3 equiv.), EPhos (36.60 mg, 0.068 mmol, 0.1 equiv.) and EPhos Pd G4 (62.86 mg, 0.068 mmol, 0.1 equiv.) in dioxane (8 mL) was stirred overnight at 80° C. under a nitrogen atmosphere. The reaction was quenched with water at room temperature. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. This resulted in N-{3-[(4-methoxyphenyl)methoxy]-1-methylindazol-4-yl}-6-[4-(trifluoromethyl)pyrazol-1-yl]pyridine-3-sulfonamide (300 mg, 78.48%) as an off-white solid.

Step 2: Synthesis of N-(1-methyl-3-oxo-2,3-dihydro-1H-indazol-4-yl)-6-(4-(trifluoromethyl)-1H-pyrazol-1-yl)pyridine-3-sulfonamide (1-198). A solution of 198-1 (100 mg, 0.179 mmol, 1 equiv.) and TFA (1.99 mL, 26.850 mmol, 150 equiv.) in DCM (2 mL) was stirred for 1 h at room temperature under a nitrogen atmosphere. The resulting mixture was concentrated under reduced pressure. The residue was purified by reversed-phase flash chromatography with the following conditions: column: C18 silica gel; mobile phase: MeCN in water (0.1% FA), 60% to 70% gradient in 10 min; detector: UV 254 nm. This resulted in N-(1-methyl-3-oxo-2H-indazol-4-yl)-6-[4-(trifluoromethyl)pyrazol-1-yl]pyridine-3-sulfonamide (62.2 mg, 78.77%) as an off-white solid. LCMS: (ES, m/z): [M+H]⁺ 439; ¹H NMR (400 MHz, DMSO-d₆) δ 3.75 (s, 3H), 6.78 (d, J7.2 Hz, 1H), 7.21-7.29 (m, 2H), 8.08 (d, J8.8 Hz, 1H), 8.31-8.41 (m, 2H), 8.80 (s, 1H), 9.27 (s, 1H), 9.88 (s, 1H), 10.78 (s, 1H).

### Example 270—Synthesis of N-(3-Methylimidazo[1,5-A]Pyridin-5-yl)-6-(4-(Trifluoromethyl)-1H-Pyrazol-1-yl)Pyridine-3-Sulfonamide (I-199)

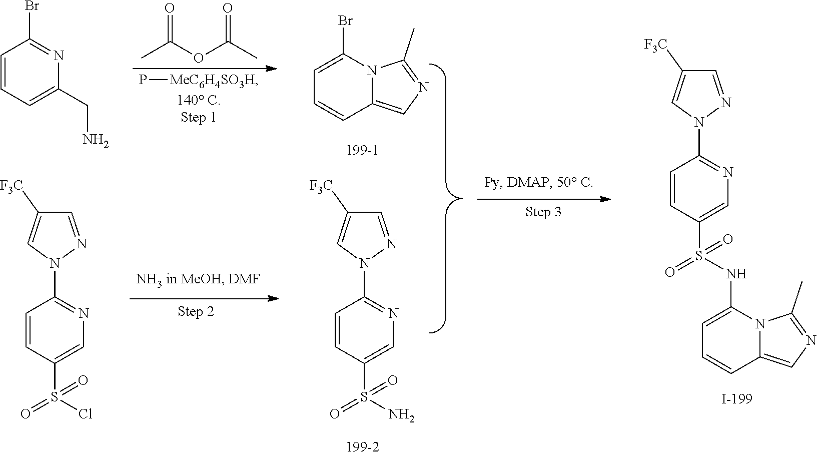

Step 1: Synthesis of 199-1. To acetic anhydride (5 mL) was added 1-(6-bromopyridin-2-yl) methanamine (2 g, 10.693 mmol, 1 equiv.) in portions at 0° C. under a nitrogen atmosphere. To the above mixture was added p-toluene sulfonic acid (2.035 g, 11.818 mmol, 1.11 equiv.) in portions at room temperature under a nitrogen atmosphere. The resulting mixture was stirred for 2 h at 140° C. under a nitrogen atmosphere. The reaction was quenched with water (20 mL). The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 30% to 50% gradient in 10 min; detector: UV 254 nm. The resulting mixture was concentrated under reduced pressure. This resulted in 5-bromo-3-methylimidazo[1,5-a]pyridine (2 g, 88.62%) as a brown solid.

Step 2: Synthesis of 199-2. To a stirred solution of 6-[4-(trifluoromethyl) pyrazol-1-yl]pyridine-3-sulfonyl chloride (500 mg, 1.604 mmol, 1 equiv.) in THE (5 mL) was added a solution of NH₃ in MeOH (1 mL, 1.000 mmol, 0.62 equiv.) dropwise at room temperature under a nitrogen atmosphere. The resulting mixture was stirred for 2 h at room temperature under a nitrogen atmosphere. The resulting mixture was extracted with EtOAc (2×20 mL). The combined organic layers were washed with brine (2×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. The resulting mixture was concentrated under reduced pressure. This resulted in 6-[4-(trifluoromethyl) pyrazol-1-yl]pyridine-3-sulfonamide (330 mg, 70.39%) as a white solid.

Step 3: Synthesis of N-(3-methylimidazo[1,5-a]pyridin-5-yl)-6-(4-(trifluoromethyl)-1H-pyrazol-1-yl)pyridine-3-sulfonamide (1-199). To a stirred mixture of 6-[4-(trifluoromethyl) pyrazol-1-yl] pyridine-3-sulfonamide (166.15 mg, 0.569 mmol, 1.2 equiv.) and 5-bromo-3-methylimidazo[1,5-a] pyridine (100 mg, 0.474 mmol, 1 equiv.) in dioxane (15 mL) were added XantPhos (54.83 mg, 0.095 mmol, 0.2 equiv.), Xantphos Pd G3 (89.87 mg, 0.095 mmol, 0.2 equiv.) and Cs₂CO₃ (463.11 mg, 1.422 mmol, 3 equiv.) in portions at room temperature under a nitrogen atmosphere. The resulting mixture was stirred for 5 h at 90° C. under a nitrogen atmosphere. The reaction was quenched with water (20 mL). The resulting mixture was extracted with EtOAc (2×50 mL). The combined organic layers were washed with brine (2×50 mL) and dried over anhydrous Na₂SO₄. The residue was purified by reversed-phase flash chromatography with the following conditions: column: C18 silica gel; mobile phase: MeCN in water (0.1% FA), 50% to 60% gradient in 10 min; detector: UV 254 nm. The resulting mixture was concentrated under reduced pressure. This resulted in N-{3-methylimidazo[1,5-a] pyridin-5-yl}-6-[4-(trifluoromethyl) pyrazol-1-yl] pyridine-3-sulfonamide (24.3 mg, 11.61%) as a yellow solid. LCMS: (ES, m/z): [M+H]⁺ 423; ¹H NMR (400 MHz, DMSO-d₆): δ 3.22 (s, 3H), 6.08-6.13 (m, 1H), 6.79-6.84 (m, 2H), 7.65 (s, 1H), 8.03 (d, J8.4 Hz, 1H), 8.33-8.40 (m, 2H), 8.85 (d, J2.0 Hz, 1H), 9.21 (s, 1H), 14.55 (s, 1H).

### Example 271—Synthesis of 6-(3-Isopropyl-1H-Pyrazol-1-yl)-N-(1-(2-Methoxyethyl)-1H-Indazol-7-yl)Pyridine-3-Sulfonamide (I-215)

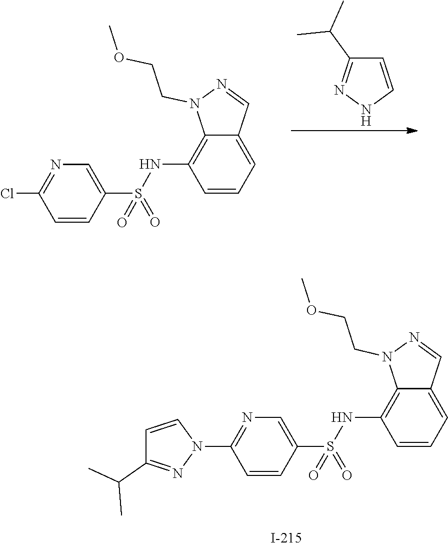

A mixture of 6-chloro-N-(1-(2-methoxyethyl)-1H-indazol-7-yl)pyridine-3-sulfonamide (200.00 mg, 0.545 mmol, 1 equiv.), 3-isopropyl-1H-pyrazole (72.07 mg, 0.654 mmol, 1.2 equiv.) and Cs₂CO₃ (532.94 mg, 1.635 mmol, 3 equiv.) in DMSO (2 mL) was stirred for 2 h at 140° C. The resulting mixture was diluted with water (10 mL). The resulting mixture was extracted with EtOAc (3×15 mL). The combined organic layers were washed with brine (20 mL) and dried over anhydrous Na₂SO₄. After filtration, the filtrate was concentrated under reduced pressure. The crude product was purified by prep-HPLC with the following conditions: Column: XBridge Prep OBD C18, 30*150 mm, 5 m; mobile phase A: water (10 mmol/L NH₄HCO₃)+0.05% NH₃. H₂O, mobile phase B: ACN; flow rate 60 mL/min; gradient: 38% B to 65% B in 7 min; wavelength: 254 nm/220 nm; Rt1(min): 8.3, to afford 6-(3-isopropyl-1H-pyrazol-1-yl)-N-(1-(2-methoxyethyl)-1H-indazol-7-yl)pyridine-3-sulfonamide (61.4 mg, 24.49%) as an off-white solid. LCMS: (ES, m/z): [M+H]⁺ 441; (400 MHz, DMSO-d₆) δ 1.24 (d, J 6.8 Hz, 6H), 2.91-3.03 (m, 1H), 3.04 (s, 3H), 3.61 (t, J 5.2 Hz, 2H), 4.45 (t, J 5.2 Hz, 2H), 6.50 (d, J 2.8 Hz, 1H), 6.97 (t, J 7.6 Hz, 1H), 7.11 (d, J 7.2 Hz, 1H), 7.49 (d, J 8.4 Hz, 1H), 7.91 (d, J 8.8 Hz, 1H), 8.29 (d, J 8.4 Hgz, 1H), 8.30 (s, 1H), 8.50 (d, J 2.8 Hz, 1H), 8.73 (d, J 2.4 Hz, 1H), 10.49 (s, 1H).

### Example 272—Synthesis of 6-(6,7-Dihydropyrano[4,3-C]Pyrazol-2(4H)-yl)-N-(1-Methyl-1H-Indazol-7-yl)Pyridine-3-Sulfonamide (I-247)

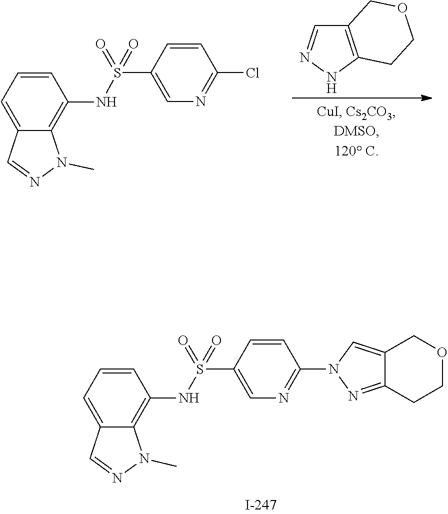 [SMILES: Cn1ncc2cccc(NS(=O)(=O)c3ccc(Cl)nc3)c21 ; c1n[nH]c2c1COCC2 ; Cn1ncc2cccc(NS(=O)(=O)c3ccc(-n4cc5c(n4)CCOC5)nc3)c21]

A solution of 6-chloro-N-(1-methylindazol-7-yl)pyridine-3-sulfonamide (300 mg, 0.929 mmol, 1 equiv.), 1H,4H,6H,7H-pyrano[4,3-c]pyrazole (288.46 mg, 2.323 mmol, 2.5 equiv.), CuI (708.06 mg, 3.716 mmol, 4 equiv.), Cs₂CO₃ (1817.00 mg, 5.574 mmol, 6 equiv.) and (1R,2R)—N1,N2-dimethylcyclohexane-1,2-diamine (264.41 mg, 1.858 mmol, 2 equiv.) in DMSO (10 mL) was stirred for 5 h at 120° C. under a nitrogen atmosphere. The reaction was quenched by the addition of water (10 mL) at 0° C. The aqueous layer was extracted with CH₂Cl₂ (2×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA), 50% to 65% gradient in 10 min; detector: UV 254 nm to afford N-(1-methylindazol-7-yl)-6-{4H,6H,7H-pyrano[4,3-c]pyrazol-2-yl}pyridine-3-sulfonamide (crude 230 mg) as a yellow solid. The residue was purified by prep-TLC (Column: CHIRALPAKIG-34.6*50 mm, 3.0 um; mobile phase A: hex(0.2% DEA): (MeOH:DCM=1:1)=60:40; gradient: isocratic; injection volume: 2.0 mL) to afford N-(1-methylindazol-7-yl)-6-{4H,6H,7H-pyrano[4,3-c]pyrazol-2-yl}pyridine-3-sulfonamide (93.2 mg, 24.19%) as a yellow solid. LCMS: (ES, m/z): [M+H]⁺ 411; (400 MHz, DMSO-d₆) δ 2.81 (t, J5.6 Hz, 2H), 3.91 (t, J5.6 Hz, 2H), 4.28 (s, 3H), 4.69 (s, 2H), 6.51 (d, J7.2 Hz, 1H), 6.94 (t, J 7.6 Hz, 1H), 7.71 (d, J 8.0 Hz, 1H), 8.04 (d, J 8.8 Hz, 1H), 8.10 (s, 1H), 8.17 (dd, J8.8, 2.4 Hz, 1H), 8.44 (s, 1H), 8.56 (d, J2.0 Hz, 1H), 10.25 (s, 1H).

### Example 273—Synthesis of 6-(2-Methyl-1-(Trifluoromethyl)-1H-Imidazol-4-yl)-N-(1-Methyl-1H-Indazol-7-yl)Pyridine-3-Sulfonamide (I-264)

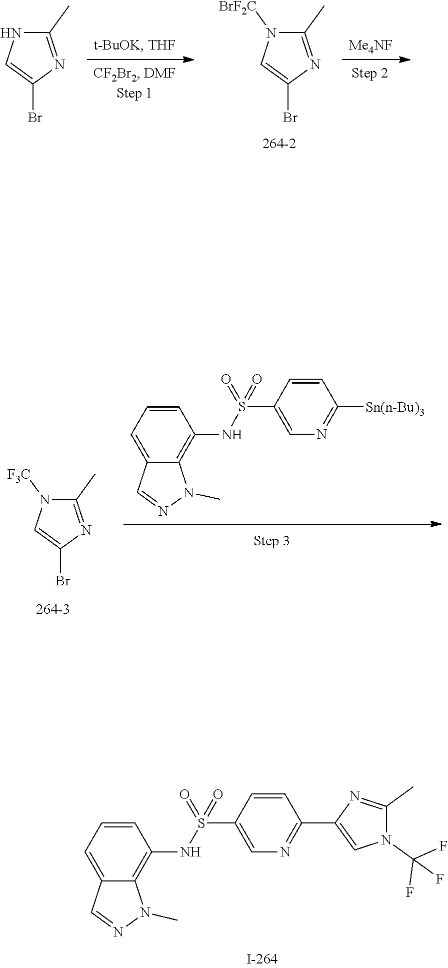

Step 1: Synthesis of 264-2: A solution of 4-bromo-2-methyl-1H-imidazole (3 g, 18.633 mmol, 1 equiv) and t-BuOK (2.30 g, 20.496 mmol, 1.1 equiv) in THE (10 mL) was stirred for 30 min at room temperature under a nitrogen atmosphere. To the above mixture was added dibromodifluoromethane (5.11 mL, 55.899 mmol, 3 equiv) and DMF (20 mL) at room temperature. The resulting mixture was stirred for an additional 4 h at room temperature. The resulting mixture was diluted with water (50 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. This resulted in 4-bromo-1-(bromodifluoromethyl)-2-methylimidazole (264-2, 850 mg, 7.87%) as a brown oil.

Step 2: Synthesis of 264-3: A solution of 4-bromo-1-(bromodifluoromethyl)-2-methylimidazole (100 mg, 0.207 mmol, 1 equiv, 60%) and tetramethylammonium fluoride (38.56 mg, 0.414 mmol, 2 equiv) in sulfolane (2 mL) was stirred overnight at 100° C. under a nitrogen atmosphere. The resulting mixture was diluted with water (10 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The crude product was purified by Prep-HPLC with the following conditions (column: XBridge Prep Phenyl OBD column, 19*250 mm, 5 m; mobile phase A: water (0.05% TFA), mobile phase B: ACN; flow rate: 25 mL/min; gradient: 27% B to 53% B in 10 min; wavelength: 254 nm/220 nm; RT1(min): 9.05 9.68) to afford 264-3 (60 mg, 14.26%) as a yellow oil.

Step 3: Synthesis of 6-(2-methyl-1-(trifluoromethyl)-1H-imidazol-4-yl)-N-(1-methyl-1H-indazol-7-yl)pyridine-3-sulfonamide (1-264): To a stirred solution of 4-bromo-2-methyl-1-(trifluoromethyl)imidazole (237.97 mg, 1.038 mmol, 3 equiv) and N-(1-methylindazol-7-yl)-6-(tributylstannyl)pyridine-3-sulfonamide (200 mg, 0.346 mmol, 1.00 equiv) in 1,4-dioxane (2 mL) were added Xphos (33.03 mg, 0.069 mmol, 0.2 equiv) and Xphos Pd G3 (58.64 mg, 0.069 mmol, 0.2 equiv) at room temperature under a nitrogen atmosphere. The resulting mixture was stirred overnight at 110° C. under a nitrogen atmosphere. The resulting mixture was diluted with water (10 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. The crude product (120 mg) was purified by Prep-HPLC with the following conditions (column: XBridge Prep Phenyl OBD column, 19*250 mm, 5 m; mobile phase A: water (0.05% TFA), mobile phase B: ACN; flow rate: 25 mL/min; gradient: 27% B to 53% B in 10 min; wavelength: 254 nm/220 nm; RT1(min): 9.05 9.68) to afford 6-[2-methyl-1-(trifluoromethyl)imidazol-4-yl]-N-(1-methylindazol-7-yl)pyridine-3-sulfonamide (I-264, 21.6 mg, 14.26%) as an off-white solid. LCMS (ES, m/z): [M+1]⁺=437; ¹H NMR (400 MHz, DMSO-d₆) δ 2.58 (s, 3H), 4.29 (s, 3H), 6.52 (d, J=7.6 Hz, 1H), 6.90 (t, J=7.6 Hz, 1H), 7.62 (d, J=8.0 Hz, 1H), 8.07 (s, 1H), 8.08 (s, 1H), 8.12 (dd, J=8.6, 2.4 Hz, 1H), 8.33 (s, 1H), 8.73 (d, J=2.4 Hz, 1H), 10.28 (s, 1H).

### Example 274—Synthesis of 6-(2-Cyclopropylthiazol-5-yl)-N-(1-Methyl-1H-Indazol-7-yl)Pyridine-3-Sulfonamide (I-265)

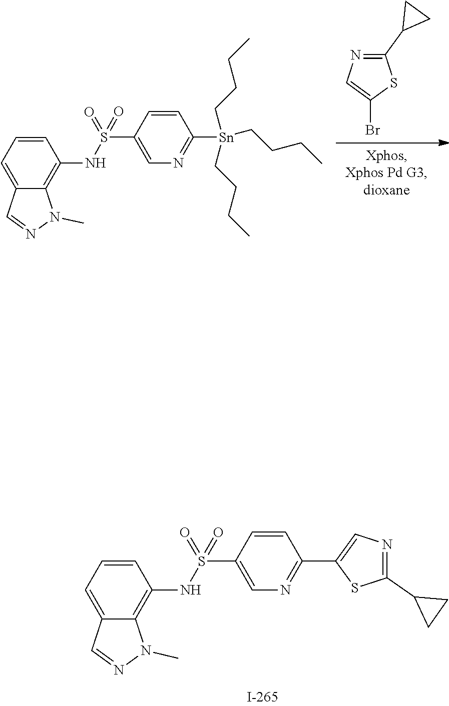 [SMILES: CCC[CH2][Sn]([CH2]CCC)([CH2]CCC)[c]1ccc(S(=O)(=O)Nc2cccc3cnn(C)c23)cn1 ; Brc1cnc(C2CC2)s1 ; Cn1ncc2cccc(NS(=O)(=O)c3ccc(-c4cnc(C5CC5)s4)nc3)c21]

A solution of N-(1-methylindazol-7-yl)-6-(tributylstannyl)pyridine-3-sulfonamide (100 mg, 0.173 mmol, 1 equiv), 5-bromo-2-cyclopropyl-1,3-thiazole (42.42 mg, 0.208 mmol, 1.2 equiv), XPhos (16.51 mg, 0.035 mmol, 0.2 equiv) and XPhos Pd G₃ (14.66 mg, 0.017 mmol, 0.1 equiv) in dioxane (4 mL) was stirred for 2 h at 90° C. under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The crude product was purified by Prep-HPLC with the following conditions (column: XBridge Prep OBD C18 column, 30*150 mm, 5 m; mobile phase A: water (10 mmol/L NH₄HCO₃⁺0.05% NH₃H₂O), mobile phase B: ACN; flow rate: 60 mL/min; gradient: 15% B to 35% B in 7 min; wavelength: 254 nm/220 nm; RT1(min): 6.82; number of runs: 3) to afford 6-(2-cyclopropyl-1,3-thiazol-5-yl)-N-(1-methylindazol-7-yl)pyridine-3-sulfonamide (I-265, 11.8 mg, 16.42%) as a yellow solid. LCMS (ES, m/z): [M+H]⁺ 412; ¹H NMR (400 MHz, chloroform-d) δ 1.14-1.19 (m, 2H), 1.20-1.28 (m, 2H), 2.34-2.41 (m, 1H), 4.41 (s, 3H), 6.36 (s, 1H), 6.57 (d, J=7.2 Hz, 1H), 6.91 (t, J=7.8 Hz, 1H), 7.24 (s, 1H), 7.68 (dd, J=15.8, 8.2 Hz, 2H), 7.87 (dd, J=8.4, 2.4 Hz, 1H), 8.01 (s, 1H), 8.14 (s, 1H), 8.81 (s, 1H).

### Example 275—Synthesis of 6-(2-(2-Hydroxypropan-2-yl)Thiazol-5-yl)-N-(1-Methyl-1H-Indazol-7-yl)Pyridine-3-Sulfonamide (I-267)

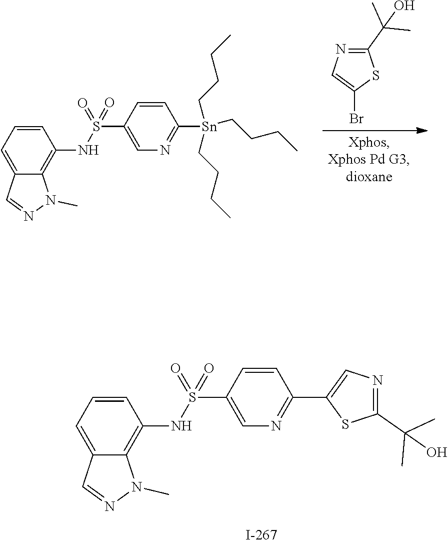

A solution of N-(1-methylindazol-7-yl)-6-(tributylstannyl)pyridine-3-sulfonamide (100 mg, 0.173 mmol, 1 equiv), 2-(5-bromo-1,3-thiazol-2-yl)propan-2-ol (46.16 mg, 0.208 mmol, 1.2 equiv), XPhos (16.51 mg, 0.035 mmol, 0.2 equiv) and XPhos Pd G₃ (14.66 mg, 0.017 mmol, 0.1 equiv) in dioxane (4 mL) was stirred for 2 h at 90° C. under a nitrogen atmosphere. The mixture was allowed to cool down to room temperature. The resulting mixture was diluted with water (20 mL). The resulting mixture was extracted with EtOAc (1×10 mL). The combined organic layers were washed with brine (1×25 mL) and dried over anhydrous Na₂SO₄. After filtration, the filtrate was concentrated under reduced pressure. The crude product was purified by Prep-HPLC with the following conditions (column: XBridge Prep OBD C18 column, 30*150 mm, 5 μm; mobile phase A: water (10 mmol/L NH₄HCO₃⁺0.05% NH₃H₂O), mobile phase B: ACN; flow rate: 60 mL/min; gradient: 12% B to 27% B in 7 min; wavelength: 254 nm/220 nm; RT1(min): 6.55; number of runs: 2) to afford 6-[2-(2-hydroxypropan-2-yl)-1,3-thiazol-5-yl]-N-(1-methylindazol-7-yl)pyridine-3-sulfonamide (1-267, 6 mg, 7.98%) as an off-white solid. LCMS (ES, m/z): [M+H]⁺ 430; ¹H NMR (400 MHz, DMSO-d₆): δ 1.53 (s, 6H), 4.29 (s, 3H), 6.11 (s, 1H), 6.55 (d, J=7.2 Hz, 1H), 6.90 (d, J=7.6 Hz, 1H), 7.57 (s, 1H), 8.05 (dd, J=8.8, 2.4 Hz, 2H), 8.13 (d, J=8.4 Hz, 1H), 8.48 (s, 1H), 8.72 (d, J=2.4 Hz, 1H), 10.29 (s, 1H).

### Example 276—Synthesis of 6-(5,6-Dihydro-4H-Pyrrolo[1,2-B]Pyrazol-3-yl)-N-(1-Methyl-1H-Indazol-7-yl)Pyridine-3-Sulfonamide (I-269

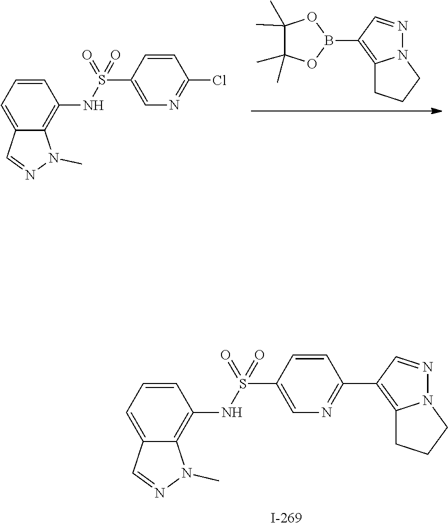 [SMILES: Cn1ncc2cccc(NS(=O)(=O)c3ccc(Cl)nc3)c21 ; CC1(C)OB(c2cnn3c2CCC3)OC1(C)C ; Cn1ncc2cccc(NS(=O)(=O)c3ccc(-c4cnn5c4CCC5)nc3)c21]

A solution of 6-chloro-N-(1-methylindazol-7-yl)pyridine-3-sulfonamide (200 mg, 0.620 mmol, 1 equiv) in dioxane (4 mL) and H₂O (1 mL) was treated with 3-(4,4,5,5-tetramethyl-1,3,2-dioxaborolan-2-yl)-4H,5H,6H-pyrrolo[1,2-b]pyrazole (174.1 mg, 0.744 mmol, 1.2 equiv) for 2 min at room temperature under a nitrogen atmosphere followed by the addition of Pd(dppf)Cl₂ (90.7 mg, 0.124 mmol, 0.2 equiv) and Cs₂CO₃ (141.5 mg, 1.860 mmol, 3 equiv) in portions at 90° C. The resulting mixture was stirred for an additional 2 h at 90° C. The resulting mixture was concentrated under reduced pressure. The residue was purified by reversed-phase flash chromatography with the following conditions: column: C18 silica gel; mobile phase: MeCN in water (0.1% FA), 0% to 50% gradient in 20 min; detector: UV 254 nm. This resulted in 6-(5,6-dihydro-4H-pyrrolo[1,2-b]pyrazol-3-yl)-N-(1-methyl-1H-indazol-7-yl)pyridine-3-sulfonamide (1-269, 84.5 mg, 34.50%) as a white solid. LCMS (ES, m/z): [M+H]⁺=395; ¹H NMR (400 MHz, DMSO-d₆) δ 2.62 (s, J=7.2 Hz, 2H), 3.14 (t, J=7.2 Hz, 2H), 4.14 (t, J=7.2 Hz, 2H), 4.28 (d, J=1.6 Hz, 3H), 6.50 (d, J=7.2 Hz, 1H), 6.93 (t, J=7.6 Hz, 1H), 7.69 (d, J=8.0 Hz, 1H), 7.74 (d, J=8.4 Hz, 1H), 7.95 (dt, J=8.0, 2.0 Hz, 1H), 8.09 (s, 1H), 8.14 (s, 1H), 8.66 (d, J=2.4 Hz, 1H), 10.19 (s, 1H).

### Example 277—Synthesis of 6-(6,7-Dihydro-4H-Pyrazolo[5,1-C][1,4]Oxazin-3-yl)-N-(1-Methyl-1H-Indazol-7-yl)Pyridine-3-Sulfonamide (I-270)

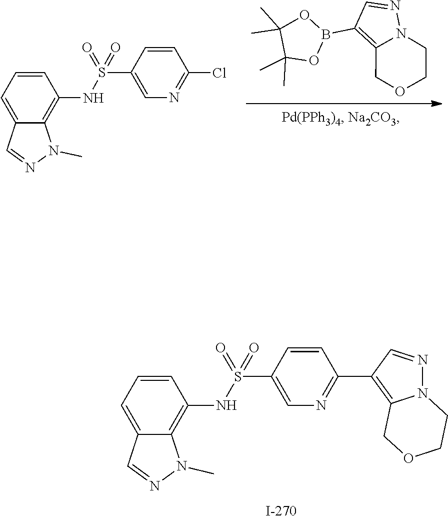 [SMILES: Cn1ncc2cccc(NS(=O)(=O)c3ccc(Cl)nc3)c21 ; CC1(C)OB(c2cnn3c2COCC3)OC1(C)C ; Cn1ncc2cccc(NS(=O)(=O)c3ccc(-c4cnn5c4COCC5)nc3)c21]

A solution of 6-chloro-N-(1-methylindazol-7-yl)pyridine-3-sulfonamide (150 mg, mmol, 1 equiv), 3-(4,4,5,5-tetramethyl-1,3,2-dioxaborolan-2-yl)-4H,6H,7H-pyrazolo[3,2-c][1,4]oxazine (139.48 mg, 0.558 mmol, 1.2 equiv), Pd(PPh₃)₄(107.41 mg, 0.093 mmol, 0.2 equiv) and Na₂CO₃ (147.77 mg, 1.395 mmol, 3 equiv) in 1,4-dioxane (10 mL) and H₂O (2 mL) was stirred for 2 h at 90° C. under a nitrogen atmosphere. The desired product could be detected by LCMS. The reaction was quenched with water at room temperature. The aqueous layer was extracted with DCM (3×100 mL). The residue was purified by reversed-phase flash chromatography with the following conditions: column: C18 silica gel; mobile phase: ACN in water (0.1% NH₃·H₂O), 0% to 100% gradient in 40 min; detector: UV 254 nm to afford 6-(6,7-dihydro-4H-pyrazolo[5,1-c][1,4]oxazin-3-yl)-N-(1-methyl-1H-indazol-7-yl)pyridine-3-sulfonamide (I-270, 66 mg, 33.91%) as a white solid. LCMS (ES, m/z): [M+H]⁺=411; ¹H NMR (400 MHz, DMSO-d₆) δ 4.09 (t, J=4.4 Hz, 2H), 4.19 (t, J=4.4 Hz, 2H), 4.30 (s, 3H), 5.12 (s, 2H), 6.54 (d, J=7.2 Hz, 1H), 6.89 (t, J=8.0 Hz, 1H), 7.55 (d, J=8.0 Hz, 1H), 7.84 (d, J=8.4 Hz, 1H), 7.95 (dd, J=8.4, 2.0 Hz, 1H), 8.03 (s, 1H), 8.22 (s, 1H), 8.69 (d, J=2.4 Hz, 1H), 10.19 (s, 1H).

### Example 278—Synthesis of N-(3-Fluoro-6-Methoxy-1-Methyl-1H-Indazol-7-yl)-6-(4-(Trifluoromethyl)-1H-Pyrazol-1-yl)Pyridine-3-Sulfonamide (I-273)

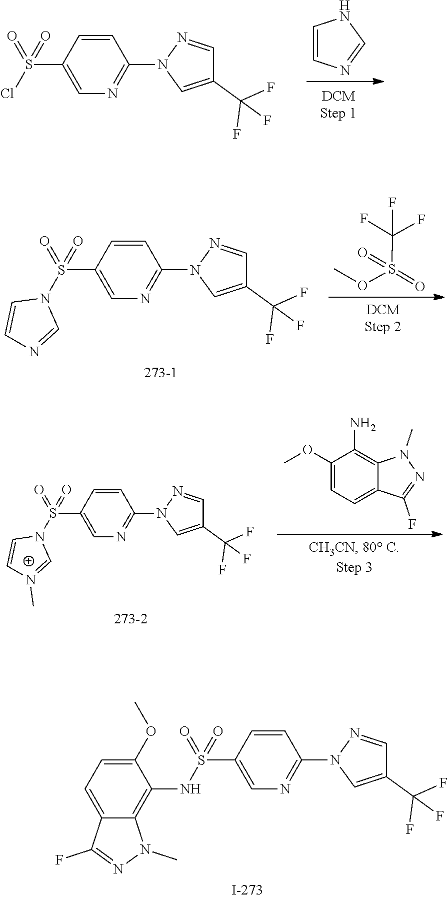

Step 1: Synthesis of 273-1: To a stirred mixture of 6-[4-(trifluoromethyl)pyrazol-1-yl]pyridine-3-sulfonyl chloride (1 g, 3.209 mmol, 1 equiv) in DCM (5 mL) were added imidazole (0.66 g, 9.627 mmol, 3 equiv) in portions at 0° C. under an air atmosphere. The resulting mixture was stirred for an additional 1 h at 0° C. The resulting mixture was filtered and the filter cake was washed with n-hexane (20×4 mL). The filtrate was concentrated under reduced pressure. This resulted in 273-1 (950 mg, 86.25%) as a yellow solid.

Step 2: Synthesis of 273-2: To a stirred mixture of 271-1 (150 mg, 0.437 mmol, 1 equiv) in DCM (10 mL) was added triflate ester (215.12 mg, 1.311 mmol, 3 equiv) in portions at 0° C. under air an atmosphere. The resulting mixture was stirred for an additional 3 h at 0° C. The resulting mixture was quenched with water (50 mL) and extracted with CH₂Cl₂ (5×20 mL). The combined organic layers were washed with brine (2×10 mL) and dried over anhydrous Na₂SO₄. The resulting mixture was concentrated under reduced pressure. This resulted in 273-2 (80 mg, 51.09%) as a yellow solid.

Step 3: Synthesis of N-(3-fluoro-6-methoxy-1-methyl-1H-indazol-7-yl)-6-(4-(trifluoromethyl)-1H-pyrazol-1-yl)pyridine-3-sulfonamide (I-273): A solution of 273-2 (50 mg, 0.256 mmol, 1 equiv) in THF (3 mL) was treated with lithiobis(trimethylsilyl)amine (171.44 mg, 1.024 mmol, 4 equiv) for 0.5 h at −78° C. under a nitrogen atmosphere followed by the addition of 6-[4-(trifluoromethyl)pyrazol-1-yl]pyridine-3-sulfonyl chloride (159.66 mg, 0.512 mmol, 2 equiv) in portions at −78° C. The resulting mixture was stirred for additional 2 h at −78° C. The residue was purified by reversed-phase flash chromatography with the following conditions: column: C18 silica gel; mobile phase: MeCN in water (10 mmol/L NH₄HCO₃), 15% to 50% gradient in 10 min; detector: UV 254 nm. The crude product (50 mg) was purified by Prep-HPLC with the following conditions (column: XBridge Prep OBD C18 column, 30*150 mm, 5 m; mobile phase A: water (10 mmol/L NH₄HCO₃)+0.05% NH₃·H₂O, mobile phase B: ACN; flow rate: 60 mL/min; gradient: 18% B to 45% B in 7 min; wavelength: 254 nm/220 nm; RT1(min): 8.07) to afford N-(3-fluoro-6-methoxy-1-methyl-1H-indazol-7-yl)-6-(4-(trifluoromethyl)-1H-pyrazol-1-yl)pyridine-3-sulfonamide (I-273, 5.9 mg, 4.89%) as a white solid. LCMS (ES, m/z): [M+1]⁺=471; ¹H NMR (400 MHz, DMSO-d₆) δ3.4(m,3H), 4.29 (s, 3H),7.59 (s, 1H), 8.02 (s, 1H), 8.18 (d, J=8.8 Hz, 1H), 8.36 (dd, J=8.6, 2.4 Hz, 1H), 8.42 (s, 1H), 8.78 (d, J=2.4 Hz, 1H), 9.33 (s, 1H), 10.55 (s, 1H).

### Example 279—Synthesis of 6-(4-(2-Hydroxypropan-2-yl)-1H-Pyrazol-1-yl)-N-(6-Methoxy-1-Methyl-1H-Indazol-7-yl)Pyridine-3-Sulfonamide (I-278)

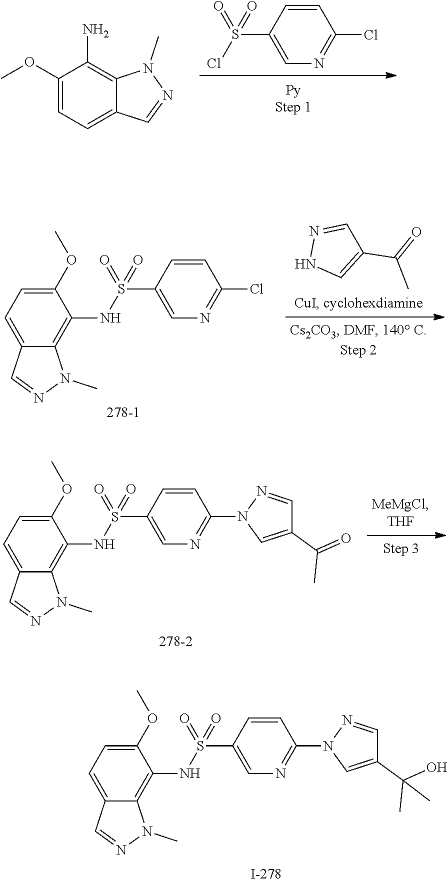

Step 1: Synthesis of 278-1: A solution of 6-methoxy-1-methylpyrazolo[4,3-c]pyridin-7-amine (531 mg, 2.980 mmol, 1 equiv) and 6-chloropyridine-3-sulfonyl chloride (758.22 mg, 3.576 mmol, 1.2 equiv) in pyridine (29 mL) was stirred overnight at room temperature. The resulting mixture was concentrated under reduced pressure to give the crude. The residue was purified by reversed-phase flash chromatography with the following conditions: column: C18 silica gel; mobile phase: ACN in water (0.1% FA), 0% to 100% gradient in 30 min; detector: UV 254 nm to give 278-1 (771 mg, 73.13%) as a yellow solid.

Step 2: Synthesis of 278-2: A solution of 278-1 (211.2 mg, 0.5 mmol, 1 equiv), cyclohexane-1,3-diamine (82.04 mg, 0.718 mmol, 1.2 equiv), 1-(1H-pyrazol-4-yl)ethanone (69.1 mg, 0.718 mmol, 1.2 equiv), CuI (57.0 mg, 0.25 mmol, 0.5 equiv) and Cs₂CO₃ (390.1 mg, 1.19 mmol, 2 equiv) in DMF (10 mL) was stirred overnight at 140° C. under a nitrogen atmosphere. After the reaction was complete, the resulting mixture was diluted with water (60 mL). The resulting mixture was extracted with EtOAc (4×50 mL). The combined organic layers were washed with brine (100 mL) and dried over anhydrous Na₂SO₄. After filtration, the filtrate was concentrated under reduced pressure to give the crude. The residue was purified by reversed-phase flash chromatography with the following conditions: column: C18 silica gel; mobile phase: ACN in water (10 mmol/L NH₄HCO₃), 0% to 100% gradient in 30 min; detector: UV 254 nm to afford 278-2 (142 mg, 55.62%) as a dark green solid.

Step 3: Synthesis of 6-(4-(2-hydroxypropan-2-yl)-1H-pyrazol-1-yl)-N-(6-methoxy-1-methyl-1H-indazol-7-yl)pyridine-3-sulfonamide (I-278): A solution of 278-2 (107.3 mg, 0.252 mmol, 1 equiv) in THF (5 mL) and methyl magnesium chloride in THF (3 M, 0.11 mL) was stirred for 1 h at 0° C. under a nitrogen atmosphere. After the reaction was complete, the reaction was quenched with sat. NH₄Cl (aq.) at room temperature. The resulting mixture was extracted with EtOAc (4×20 mL). The combined organic layers were washed with brine (50 mL) and dried over anhydrous Na₂SO₄. After filtration, the filtrate was concentrated under reduced pressure to give the crude. The crude product was purified by Prep-HPLC with the following conditions (column: XBridge Prep OBD C₁₈ column, 19*250 mm, 5 m; mobile phase A: water (10 mmol/L NH₄HCO₃+0.05% NH₃H₂O), mobile phase B: MeOH; flow rate: 25 mL/min; gradient: 38% B to 61% B in 10 min; wavelength: 254 nm/220 nm; RT₁(min): 9.63; number of runs: 6) to afford 6-(4-(2-hydroxypropan-2-yl)-1H-pyrazol-1-yl)-N-(6-methoxy-1-methyl-1H-indazol-7-yl)pyridine-3-sulfonamide (I-278, 7.5 mg, 0.66%) as an off-white solid. LCMS (ES, m/z): [M+1]⁺=443; ¹H NMR (400 MHz, DMSO-d₆) δ 1.47 (s, 6H), 3.19 (s, 3H), 4.28 (s, 3H), 5.08 (s, 1H), 6.82 (d, J=8.8 Hz, 1H), 7.64 (d, J=8.8 Hz, 1H), 7.87 (s, 1H), 7.98 (s, 1H), 8.05 (d, J=8.8 Hz, 1H), 8.16-8.19 (m, 1H), 8.49 (s, 1H), 8.58 (d, J=2.0 Hz, 1H), 10.01 (s, 1H).

### Example 280—Synthesis of 6-(4-(1-Hydroxyethyl)-1H-Pyrazol-1-yl)-N-(6-Methoxy-1-Methyl-1H-Indazol-7-yl)Pyridine-3-Sulfonamide (1-280) and 6-(4-(1-Hydroxyethyl)-1H-Pyrazol-1-yl)-N-(6-Methoxy-1-Methyl-1H-Indazol-7-yl)Pyridine-3-Sulfonamide (I-281

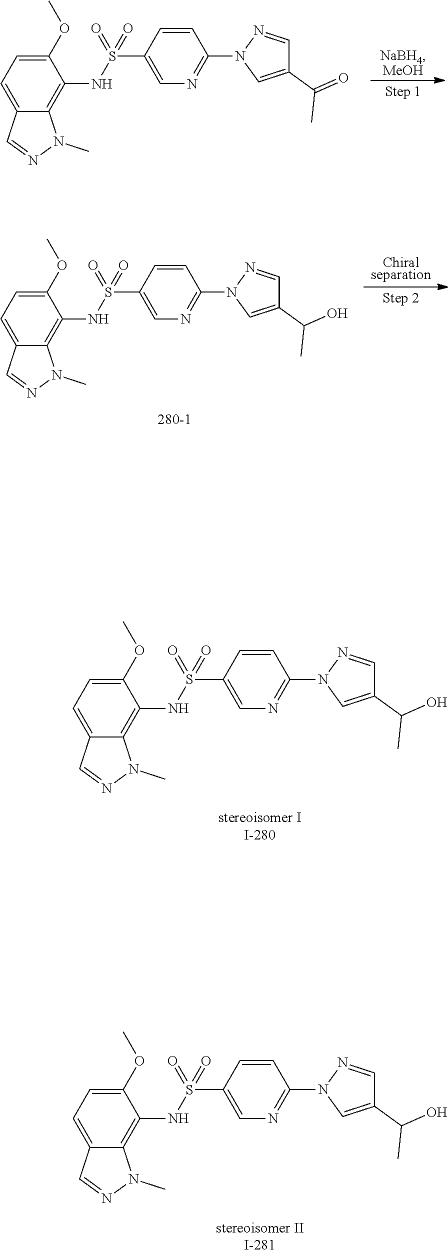

Step 1: Synthesis of 280-1: A mixture of 6-(4-acetylpyrazol-1-yl)-N-(6-methoxy-1-methylindazol-7-yl)pyridine-3-sulfonamide (140 mg, 0.328 mmol, 1 equiv) and NaBH₄ (40 mg, 1.057 mmol, 3.22 equiv) in MeOH (5 mL) was stirred for 3 h at room temperature under a nitrogen atmosphere. The residue was purified by reversed-phase flash chromatography with the following conditions: column: C18 silica gel; mobile phase: MeCN in water (10 mmol/L NH₄HCO₃), 30%-40% gradient in 10 min; detector: UV 254 nm. This resulted in 6-[4-(1-hydroxyethyl)pyrazol-1-yl]-N-(6-methoxy-1-methylindazol-7-yl)pyridine-3-sulfonamide (280-1, 55 mg, 39.10%) as an off-white solid.

Step 2: Synthesis of 6-(4-(1-hydroxyethyl)-1H-pyrazol-1-yl)-N-(6-methoxy-1-methyl-1H-indazol-7-yl)pyridine-3-sulfonamide (I-280) and 6-(4-(1-hydroxyethyl)-1H-pyrazol-1-yl)-N-(6-methoxy-1-methyl-1H-indazol-7-yl)pyridine-3-sulfonamide (I-281): 6-[4-(1-hydroxyethyl)pyrazol-1-yl]-N-(6-methoxy-1-methylindazol-7-yl)pyridine-3-sulfonamide (90 mg, 0.210 mmol, 1 equiv) was purified by Prep-HPLC with the following conditions (column: CHIRALPAKIA-34.6*50 mm, 3.0 um; mobile phase A: hex (0.2% DEA): IPA=60:40; temperature: 25; gradient: isocratic; injection volume: 8.0 mL) to afford (S)-6-(4-(1-hydroxyethyl)-1H-pyrazol-1-yl)-N-(6-methoxy-1-methyl-1H-indazol-7-yl)pyridine-3-sulfonamide (I-280, 39.6 mg) as an off-white solid and (R)-6-(4-(1-hydroxyethyl)-1H-pyrazol-1-yl)-N-(6-methoxy-1-methyl-1H-indazol-7-yl)pyridine-3-sulfonamide (I-281, 41.8 mg) as an off-white solid. LCMS (ES, m/z): [M+H]⁺=429; ¹H NMR (400 MHz, methanol-d₄) δ 1.51 (t, J=6.4 Hz, 3H), 3.24 (d, J=5.8 Hz, 3H), 4.35 (d, J=5.8 Hz, 3H), 4.90 (d, J=6.4 Hz, 1H), 6.76 (t, J=7.8 Hz, 1H), 7.64 (t, J=7.8 Hz, 1H), 7.80 (d, J=5.8 Hz, 1H), 7.92 (d, J=6.0 Hz, 1H), 8.00 (t, J=7.6 Hz, 1H), 8.09 (t, J=7.0 Hz, 1H), 8.56 (dd, J=11.2, 5.7 Hz, 2H); LCMS (ES, m/z): [M+H]⁺=429; ¹H NMR (400 MHz, methanol-d₄) δ1.52 (d, J=6.8 Hz, 3H), 3.26 (s, 3H), 4.37 (d, J=4.2 Hz, 3H), 4.90 (d, J=6.4 Hz, 1H), 6.79 (d, J=9.0 Hz, 1H), 7.66 (d, J=8.8 Hz, 1H), 7.81 (s, 1H), 7.94 (s, 1H), 8.02 (d, J=8.8 Hz, 1H), 8.10 (dd, J=8.8, 2.4 Hz, 1H), 8.61-8.54 (m, 2H).

### Example 281—Synthesis of 6-(4-(Hydroxymethyl)-1H-Pyrazol-1-yl)-N-(6-Methoxy-1-Methyl-1H-Indazol-7-yl)Pyridine-3-Sulfonamide (I-284)

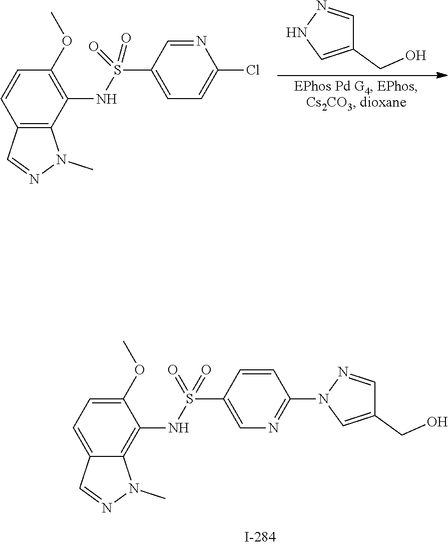

A solution of 6-chloro-N-(6-methoxy-1-methylindazol-7-yl)pyridine-3-sulfonamide (80 mg, 0.227 mmol, 1 equiv), 1H-pyrazol-4-ylmethanol (66.74 mg, 0.681 mmol, 3 equiv), EPhos (12.13 mg, 0.023 mmol, 0.1 equiv), EPhos Pd G4 (20.83 mg, 0.023 mmol, 0.1 equiv) and Cs₂CO₃ (221.65 mg, 0.681 mmol, 3 equiv) in 1,4-dioxane (5 mL) was stirred overnight at 80° C. under a nitrogen atmosphere. The reaction was quenched with water at room temperature. The resulting mixture was extracted with EtOAc (2×10 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 6-[4-(hydroxymethyl)pyrazol-1-yl]-N-(6-methoxy-1-methylindazol-7-yl)pyridine-3-sulfonamide (I-284, 20.3 mg, 21.60%) as a white solid. LCMS (ES, m/z): [M+H]⁺=415; ¹H NMR (400 MHz, DMSO-d₆) δ 3.18 (s, 3H), 4.27 (s, 3H), 4.47 (d, J=5.6 Hz, 2H), 5.09 (t, J=5.6 Hz, 1H), 6.83 (d, J=8.8 Hz, 1H), 7.68 (d, J=8.8 Hz, 1H), 7.86 (s, 1H), 7.99 (s, 1H), 8.07 (d, J=8.8 Hz, 1H), 8.17 (dd, J=8.8, 2.4 Hz, 1H), 8.54-8.62 (m, 2H), 10.02 (s, 1H).

### Example 282—Synthesis of 6-(4-(Tert-Butyl)-1H-Pyrazol-1-yl)-N-(6-Methoxy-1-Methyl-1H-Indazol-7-yl)Pyridine-3-Sulfonamide (I-285)

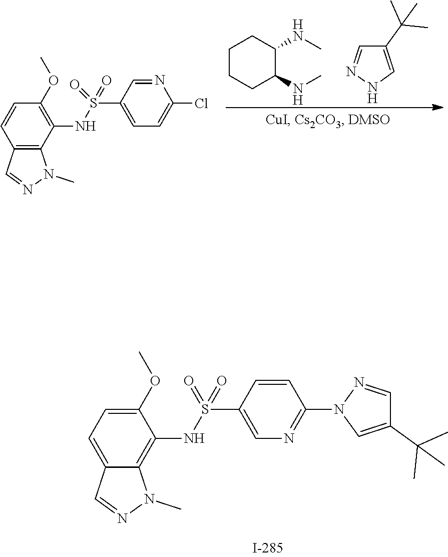

A solution/mixture of 6-chloro-N-(6-methoxy-1-methylindazol-7-yl)pyridine-3-sulfonamide (80 mg, 0.227 mmol, 1 equiv), (1R,2R)—N1,N2-dimethylcyclohexane-1,2-diamine (16.13 mg, 0.114 mmol, 0.5 equiv) 4-tert-butyl-1H-pyrazole (84.48 mg, 0.681 mmol, 3 equiv), CuI (43.19 mg, 0.227 mmol, 1 equiv) and Cs₂CO₃ (221.65 mg, 0.681 mmol, 3 equiv) in DMSO (5 mL) was stirred overnight at 120° C. under a nitrogen atmosphere. The reaction was quenched with water at room temperature. The resulting mixture was extracted with EtOAc (2×10 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 6-(4-tert-butylpyrazol-1-yl)-N-(6-methoxy-1-methylindazol-7-yl)pyridine-3-sulfonamide (I-285, 65.7 mg, 65.77%) as a white solid. LCMS (ES, m/z): [M+H]⁺=441; ¹H NMR (400 MHz, DMSO-d₆): δ 1.29 (s, 9H), 3.20 (s, 3H), 4.27 (s, 3H), 6.83 (d, J=9.0 Hz, 1H), 7.68 (d, J=8.8 Hz, 1H), 7.91 (d, J=1.0 Hz, 1H), 8.00 (s, 1H), 8.02-8.09 (m, 1H), 8.18 (dd, J=8.8, 2.4 Hz, 1H), 8.45 (d, J=1.0 Hz, 1H), 8.54-8.59 (m, 1H), 10.02 (s, 1H).

### Example 283—Synthesis of N-(6-Methoxy-1-Methyl-1H-Indazol-7-yl)-6-(4-(Trifluoromethyl)-1H-Imidazol-1-yl)Pyridine-3-Sulfonamide (I-286)

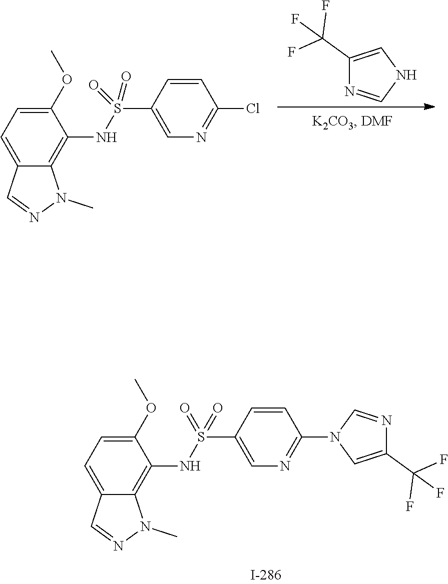

A solution of 6-chloro-N-(6-methoxy-1-methylindazol-7-yl) pyridine-3-sulfonamide (200 mg, 0.567 mmol, 1 equiv) and 4-(trifluoromethyl)-1H-imidazole (800 mg, 5.879 mmol, 10.37 equiv) and K₂CO₃ (250 mg, 1.809 mmol, 3.19 equiv) in DMSO (3 mL) was stirred for 2 h at 120° C. under a nitrogen atmosphere. The resulting mixture was diluted with water (30 mL). The resulting mixture was extracted with EtOAc (3×100 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 25% to 65% gradient in 20 min; detector: UV 254 nm. This resulted in N-(6-methoxy-1-methylindazol-7-yl)-6-[4-(trifluoromethyl)imidazol-1-yl]pyridine-3-sulfonamide (I-286, 52.7 mg, 20.49%) as an off-white solid. LCMS (ES, m/z): [M+1]⁺=453; ¹H NMR (400 MHz, DMSO-d₆) δ3.19 (s, 3H), 4.28 (s, 3H), 6.83 (d, J=8.8 Hz, 1H), 7.68 (d, J=8.8 Hz, 1H), 8.00 (s, 1H), 8.14 (d, J=8.6 Hz, 1H), 8.28 (dd, J=8.6, 2.4 Hz, 1H), 8.76-8.68 (m, 1H), 8.85 (s, 2H).

### Example 284—Synthesis of N-(6-Methoxy-1-(Methyl-D3)-1H-Pyrazolo[4,3-C]Pyridin-7-yl)-6-(4-(Trifluoromethyl)-1H-Pyrazol-1-yl)Pyridine-3-Sulfonamide (I-290)

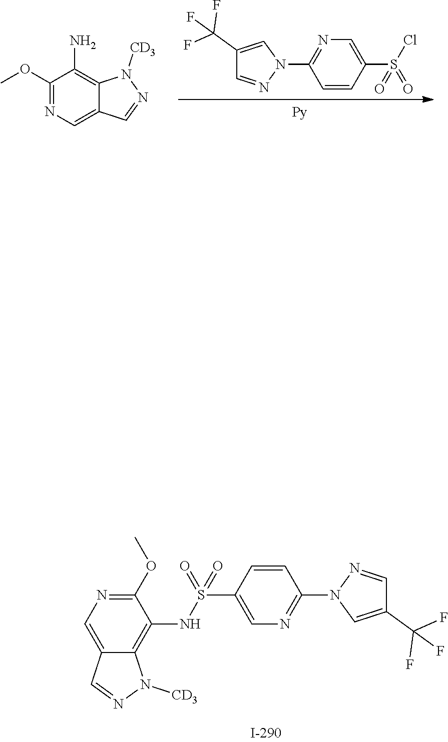

A solution of 6-methoxy-1-(methyl-d3)-1H-pyrazolo[4,3-c]pyridin-7-amine (150 mg, 0.828 mmol, 1 equiv) and 6-[4-(trifluoromethyl)pyrazol-1-yl]pyridine-3-sulfonyl chloride (644.95 mg, 2.070 mmol, 2.5 equiv) in pyridine (10 mL) was stirred overnight at room temperature. The resulting mixture was concentrated under vacuum. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 0% to 100% gradient in 20 min; detector: UV 254 nm. The crude product (75 mg) was purified by Prep-HPLC with the following conditions (column: Xselect CSH C18 OBD column 30*150 mm 5 m; mobile phase A: water(0.1% FA), mobile phase B: ACN; flow rate: 60 mL/min; gradient: 45% B to 60% B in 7 min; wavelength: 254 nm/220 nm; RT1(min): 6.5) to afford N-(6-methoxy-1-(methyl-d3)-1H-pyrazolo[4,3-c]pyridin-7-yl)-6-(4-(trifluoromethyl)-1H-pyrazol-1-yl)pyridine-3-sulfonamide (1-290, 44.6 mg, 11.62%) as a white solid. LCMS (ES, m/z): [M+1]⁺=457; ¹H NMR (400 MHz, DMSO-d₆) δ 3.27 (s, 3H), 8.15 (d, J=8.4 Hz 1H), 8.19 (s, 1H), 8.27 (dd, J=8.6, 2.4 Hz, 1H), 8.40 (s, 1H), 8.58 (s, 1H), 8.68 (d, J=2.3 Hz, 1H), 9.32 (s, 1H), 10.55 (s, 1H).

### Example 285—Synthesis of N-(6-Methoxy-1-(Methyl-D3)-1H-Pyrazolo[4,3-C]Pyridin-7-yl)-N-Methyl-6-(4-(Trifluoromethyl)-1H-Pyrazol-1-yl)Pyridine-3-Sulfonamide (I-291)

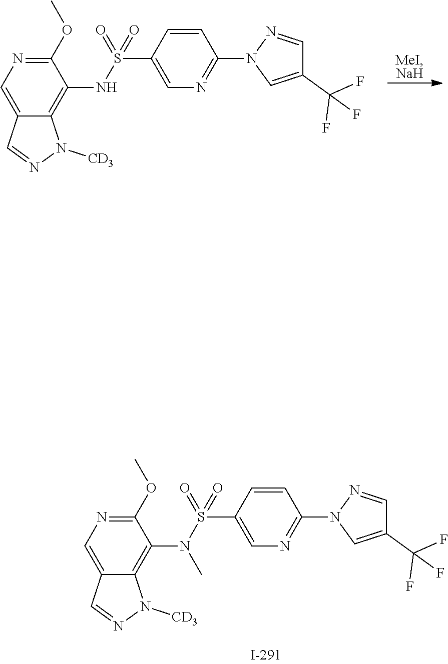

To a solution of N-(6-methoxy-1-(methyl-d3)-1H-pyrazolo[4,3-c]pyridin-7-yl)-6-(4-(trifluoromethyl)-1H-pyrazol-1-yl)pyridine-3-sulfonamide (100 mg, 0.219 mmol, 1 equiv) in DMF (2 mL) was added sodium hydride (60% in oil, 15.77 mg) at 0° C. The mixture was stirred for 15 min. Mel (46.65 mg, 0.329 mmol, 1.5 equiv) was added and the mixture was allowed to warm to rt and stirred for 1.5 h. The reaction was quenched by the addition of sat. NH₄Cl (aq.) (30 mL) at room temperature. The resulting mixture was extracted with CH₂Cl₂ (3×10 mL). The combined organic layers were washed with sat. NaCl (1×20 mL) and dried over anhydrous Na₂SO₄. After filtration, the filtrate was concentrated under reduced pressure. The residue was purified by Prep-HPLC with the following conditions (column: Xselect CSH C18 OBD column 30*150 mm 5 m; mobile phase A: water (0.1% FA), mobile phase B: ACN; flow rate: 60 mL/min; gradient: 53% B to 65% B in 7 min; wavelength: 254 nm/220 nm; RT1(min): 6.4) to afford N-(6-methoxy-1-(methyl-d3)-1H-pyrazolo[4,3-c]pyridin-7-yl)-N-methyl-6-(4-(trifluoromethyl)-1H-pyrazol-1-yl)pyridine-3-sulfonamide (1-291.15 mg, 14.54%) as a white solid. LCMS (ES, m/z): [M+H]⁺=471; ¹H NMR (400 MHz, CD₃CN) δ 3.36 (s, 3H), 3.42 (s, 3H), 8.13 (d, J=11.2 Hz, 2H), 8.18-8.25 (m, 2H), 8.67 (s, 1H), 8.74 (d, J=2.0 Hz, 1H), 9.07 (s, 1H),

### Example 286—Synthesis of 1-((6-(4-(Trifluoromethyl)-1H-Pyrazol-1-yl)Pyridin-3-yl)Sulfonyl)-1,2,3,4-Tetrahydro-[1,4]Diazepino[3,2,1-HI]Indazole (I-292)

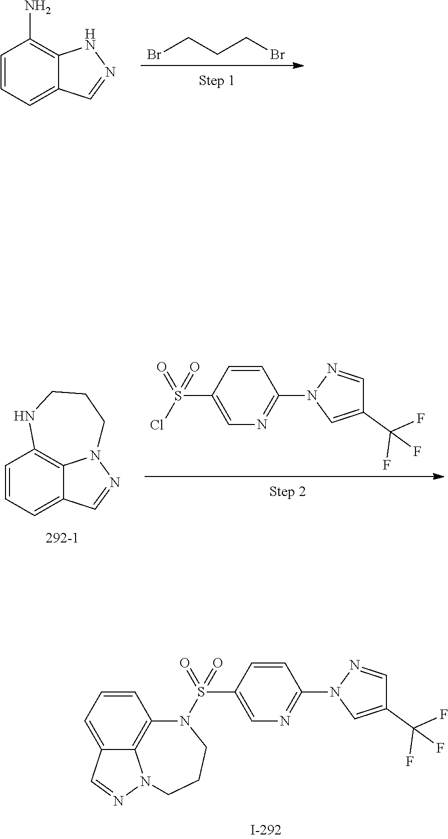

Step 1: Synthesis of 292-1: To a stirred mixture of 1H-indazol-7-amine (1 g, 7.510 mmol, 1 equiv) and 1,3-dibromopropane (2.27 g, 11.265 mmol, 1.5 equiv) in DMF (20 mL) were added DIEA (2.43 g, 18.775 mmol, 2.5 equiv) in portions at 80° C. under an air atmosphere. The resulting mixture was stirred overnight at 80° C. The residue was purified by reversed-phase flash chromatography with the following conditions: column: C18 silica gel; mobile phase: MeCN in water (10 mmol/L NH₄HCO₃), 20% to 40% gradient in 10 min; detector: UV 254 nm. The residue was purified by silica gel column chromatography, eluted with PE/EA (5:1) to afford 292-1 (30 mg, 2.31%) as a yellow solid.

Step 2: Synthesis of 1-((6-(4-(trifluoromethyl)-1H-pyrazol-1-yl)pyridin-3-yl)sulfonyl)-1,2,3,4-tetrahydro-[1,4]diazepino[3,2,1-hi]indazole (I-292): To a stirred mixture of 292-1 (30 mg, 0.173 mmol, 1 equiv) in pyridine (1 mL) was added 6-[4-(trifluoromethyl)pyrazol-1-yl]pyridine-3-sulfonyl chloride (80.97 mg, 0.259 mmol, 1.5 equiv) in portions at room temperature under a nitrogen atmosphere. The resulting mixture was stirred for an additional 2 h at room temperature. The crude product (30 mg) was purified by Prep-HPLC with the following conditions (column: Xselect CSH C18 OBD column 30*150 mm 5 m; mobile phase A: water (0.05% TFA), mobile phase B: ACN; flow rate: 60 mL/min; gradient: 55% B to 65% B in 7 min; wavelength: 254 nm/220 nm; RT1(min): 6.1) to afford 1-((6-(4-(trifluoromethyl)-1H-pyrazol-1-yl)pyridin-3-yl)sulfonyl)-1,2,3,4-tetrahydro-[1,4]diazepino[3,2,1-hi]indazole (I-292, 1.5 mg, 1.92%) as a light yellow solid. LCMS (ES, m/z): [M+1]=449; ¹H NMR (400 MHz, DMSO-d₆) 2.19-2.29 (m, 2H), 4.12-4.24 (m, 4H), 7.17 (t, J=7.8 Hz, 1H), 7.39-7.45 (m, 1H), 7.69-7.76 (m, 1H), 8.07 (d, J=8.8 Hz, 1H), 8.12 (s, 1H), 8.29 (dd, J=8.6, 2.6 Hz, 1H), 8.39 (s, 1H), 8.68 (d, J=2.4 Hz, 1H), 9.24 (s, 1H).

### Example 287—Synthesis of 1-((6-(4-(Trifluoromethyl)-1H-Pyrazol-1-yl)Pyridin-3-yl)Sulfonyl)-2,3,4,5-Tetrahydro-1H-[1,4]Diazocino[3,2,1-HI]Indazole (I-293)

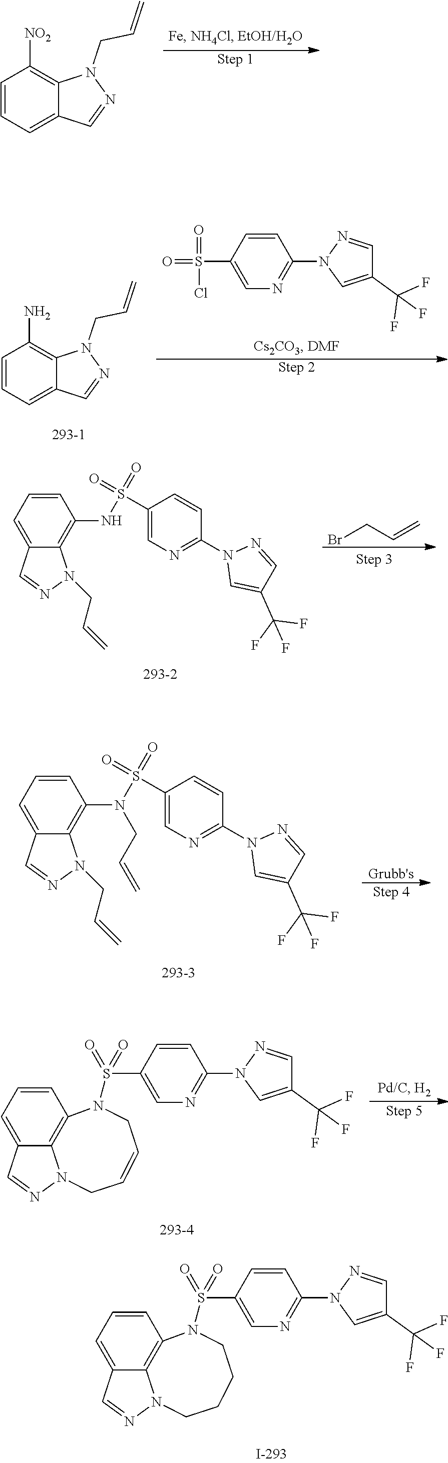

Step 1: Synthesis of 293-1: To a stirred mixture of 7-nitro-1-(prop-2-en-1-yl)indazole (1 g, 4.921 mmol, 1 equiv) and NH₄Cl (1.32 g, 24.605 mmol, 5 equiv) in EtOH (12 mL) and H₂O (3 mL) was added Fe (1.37 g, 24.605 mmol, 5 equiv) in portions at 80° C. under an air atmosphere. The resulting mixture was stirred for an additional 2 h at 80° C. The resulting mixture was filtered and the filter cake was washed with EtOAc (15×3 mL). The filtrate was concentrated under reduced pressure. The residue was purified by reversed-phase flash chromatography with the following conditions: column: C18 silica gel; mobile phase: MeCN in water (10 mmol/L NH₄HCO₃), 20% to 50% gradient in 10 min; detector: UV 254 nm. This resulted in 293-1 (470 mg, 55.14%) as a brown yellow oil.

Step 2: Synthesis of 293-2: To a stirred mixture of 293-1 (770 mg, 4.445 mmol, 1 equiv) in pyridine (10 mL) was added 6-[4-(trifluoromethyl)pyrazol-1-yl]pyridine-3-sulfonyl chloride (1.66 g, 5.334 mmol, 1.2 equiv) in portions at room temperature under an air atmosphere. The resulting mixture was stirred overnight at room temperature. The residue was purified by reversed-phase flash chromatography with the following conditions: column: C18 silica gel; mobile phase: MeCN in water (10 mmol/L NH₄HCO₃), 25% to 45% gradient in 10 min; detector: UV 254 nm. This resulted in 293-2 (435 mg, 21.82%) as a yellow solid.

Step 3: Synthesis of 293-3: To a stirred mixture of 293-2 (435 mg, 0.970 mmol, 1 equiv) and allyl bromide (586.78 mg, 4.850 mmol, 5 equiv) in DMF (5 mL) were added KI (16.10 mg, 0.097 mmol, 0.1 equiv) and K₂CO₃ (402.21 mg, 2.910 mmol, 3 equiv) in portions at room temperature under an air atmosphere. The resulting mixture was stirred for an additional 3 h at room temperature. The residue was purified by reversed-phase flash chromatography with the following conditions: column: C18 silica gel; mobile phase: MeCN in water (10 mmol/L NH₄HCO₃), 15% to 45% gradient in 10 min; detector: UV 254 nm. This resulted in 293-3 (300 mg, 63.31%) as a brown yellow solid.

Step 4: Synthesis of 293-4: To a stirred mixture of 293-3 (300 mg, 0.614 mmol, 1 equiv) in THE (10 mL) were added Grubbs 2^{nd} generation catalyst (52.14 mg, 0.061 mmol, 0.1 equiv) in portions at 50° C. under a nitrogen atmosphere. The resulting mixture was stirred overnight at 50° C. The residue was purified by reversed-phase flash chromatography with the following conditions: column: C18 silica gel; mobile phase: MeCN in water (10 mmol/L NH₄HCO₃), 20% to 40% gradient in 10 min; detector: UV 254 nm. This resulted in 293-4 (47 mg, 16.62%) as a yellow solid.

Step 5: Synthesis of 1-((6-(4-(trifluoromethyl)-1H-pyrazol-1-yl)pyridin-3-yl)sulfonyl)-2,3,4,5-tetrahydro-1H-[1,4]diazocino[3,2,1-hi]indazole (I-293): To a stirred mixture of 293-4 (65 mg, 0.141 mmol, 1 equiv) in MeOH (10 mL) was added Pd/C (30.05 mg, 0.282 mmol, 2 equiv) in portions at room temperature under a nitrogen atmosphere. The precipitated solids were collected by filtration and washed with MeOH (5×5 mL). The crude product (65 mg) was purified by Prep-HPLC with the following conditions (column: Xbridge Prep OBD C18 column, 30*150 mm, 5 μm; mobile phase A: water (10 mmol/L NH₄HCO₃)+0.05% NH₃·H₂O), mobile phase B: ACN; flow rate: 60 mL/min; gradient: 43% B to 70% B in 7 min; wavelength: 254 nm/220 nm; RT1(min): 7.8) to afford 1-((6-(4-(trifluoromethyl)-1H-pyrazol-1-yl)pyridin-3-yl)sulfonyl)-2,3,4,5-tetrahydro-1H-[1,4]diazocino[3,2,1-hi]indazole (I-293, 20.5 mg, 31.18%) as a white solid. LCMS (ES, m/z): [M+1]⁺=463; ¹H NMR (400 MHz, DMSO-d₆) δ 0.85-1.29 (m, 1H), 1.73-1.97 (m, 2H), 2.07 (s, 1H), 3.33 (s, 1H), 4.41 (t, J=11.8 Hz, 1H), 4.51-5.08 (m, 2H), 6.80-7.19 (m, 2H), 7.80 (d, J=8.2 Hz, 1H), 8.12 (s, 1H), 8.25 (d, J=8.8 Hz, 1H), 8.45 (s, 1H), 8.55 (d, J=8.8 Hz, 1H), 8.96 (s, 1H), 9.35 (s, 1H).

### Example 288—Synthesis of 1-((6-(4-(Trifluoromethyl)-1H-Pyrazol-1-yl)Pyridin-3-yl)Sulfonyl)-2,3-Dihydro-1H-Pyrazolo[1,5,4-DE]Quinoxaline (1-294)

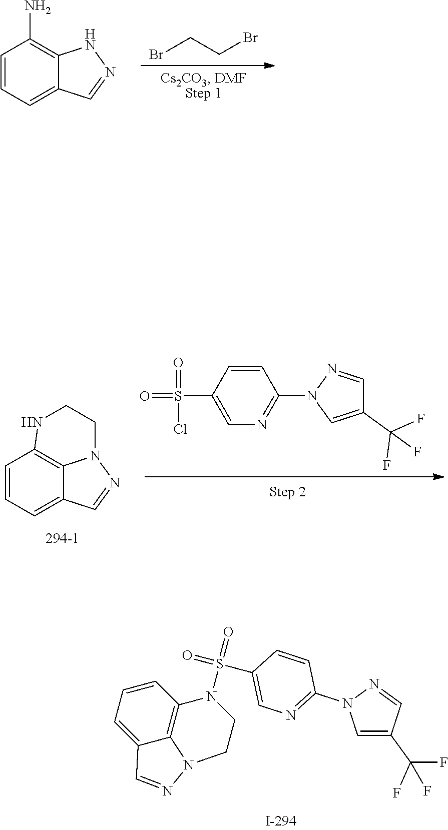

Step 1: Synthesis of 294-1: A solution of 1H-indazol-7-amine (5 g, 37.551 mmol, 1 equiv), dibromoethane (8.47 g, 45.061 mmol, 1.2 equiv) and Cs₂CO₃ (30.59 g, 93.877 mmol, 2.5 equiv) in DMF (50 mL) was stirred overnight at room temperature under a nitrogen atmosphere. The resulting mixture was diluted with water (50 mL). The resulting mixture was extracted with EtOAc (3×100 mL). The combined organic layers were washed with brine (3×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 30% to 55% gradient in 20 min; detector: UV 254 nm. This resulted in 1,2,9-triazatricyclo[6.3.1.0\hat{ }{4,12}]dodeca-2,4,6,8(12)-tetraene (294-1, 380 mg, 6.36%) as a yellow solid.

Step 2: Synthesis of 1-((6-(4-(trifluoromethyl)-1H-pyrazol-1-yl)pyridin-3-yl)sulfonyl)-2,3-dihydro-1H-pyrazolo[1,5,4-de]quinoxaline (1-294): A solution of 1,2,9-triazatricyclo[6.3.1.0\hat{ }{4,12}]dodeca-2,4,6,8(12)-tetraene (294-1, 50 mg, 0.314 mmol, 1 equiv) and 6-[4-(trifluoromethyl)pyrazol-1-yl]pyridine-3-sulfonyl chloride (100 mg, 0.321 mmol, 1.02 equiv) in pyridine (3 mL) was stirred overnight at room temperature under a nitrogen atmosphere. The desired product could be detected by LCMS. The resulting liquid was dried by lyophilization. The residue was purified by Prep-TLC (PE/EA 1:1) to afford 1-((6-(4-(trifluoromethyl)-1H-pyrazol-1-yl)pyridin-3-yl)sulfonyl)-2,3-dihydro-1H-pyrazolo[1,5,4-de]quinoxaline (1-294, 25 mg, 18.32%) as an off-white solid. LCMS (ES, m/z): [M+1]⁺=435; ¹H NMR (400 MHz, DMSO-d₆) δ 4.29 (q, J=4.8, 4.4 Hz, 2H),4.37 (dd, J=6.2, 3.8 Hz, 2H), 7.17 (t, J=7.8 Hz, 1H), 7.56 (dd, J=7.8, 2.4 Hz, 2H), 8.10-8.01 (m, 2H), 8.44-8.34 (m, 2H), 8.88 (d, J=2.4 Hz, 1H), 9.21 (s, 1H).

### Example 289—Synthesis of 6-(4-(Trifluoromethyl)-1H-Pyrazol-1-yl)-N-(1-Vinyl-1H-Indazol-7-yl)Pyridine-3-Sulfonamide (I-296)

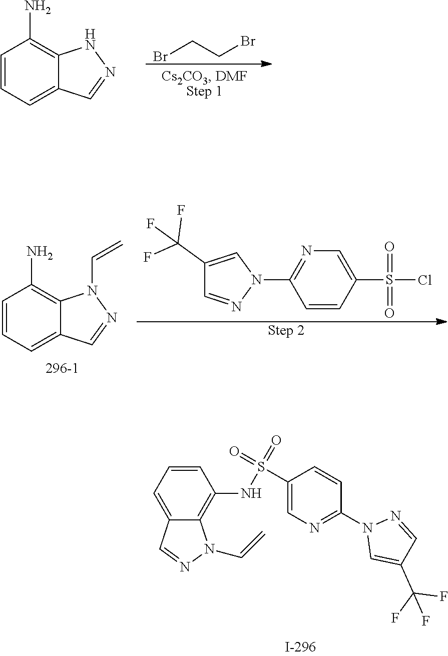

Step 1: Synthesis of 296-1: A solution of 1H-indazol-7-amine (5 g, 37.551 mmol, 1 equiv) and dibromoethane (8.47 g, 45.061 mmol, 1.2 equiv) and Cs₂CO₃ (30.59 g, 93.877 mmol, 2.5 equiv) in DMF (50 mL) was stirred overnight at room temperature under a nitrogen atmosphere. The resulting mixture was diluted with water (50 mL). The resulting mixture was extracted with EtOAc (3×100 mL). The combined organic layers were washed with brine (3×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 30% to 50% gradient in 10 min; detector: UV 254 nm. This resulted in 1-vinyl-1H-indazol-7-amine (296-1, 380 mg, 6.36%) as a yellow solid.

Step 2: Synthesis of 6-(4-(trifluoromethyl)-1H-pyrazol-1-yl)-N-(1-vinyl-1H-indazol-7-yl)pyridine-3-sulfonamide (1-296): A solution of 1-vinyl-1H-indazol-7-amine (50 mg, 0.314 mmol, 1 equiv) and 6-[4-(trifluoromethyl)pyrazol-1-yl]pyridine-3-sulfonyl chloride (100 mg, 0.321 mmol, 1.02 equiv) in pyridine (3 mL) was stirred overnight at room temperature under a nitrogen atmosphere. The desired product could be detected by LCMS. The resulting liquid was dried by lyophilization. The residue was purified by Prep-TLC (PE/EA 1:1) to afford 6-(4-(trifluoromethyl)-1H-pyrazol-1-yl)-N-(1-vinyl-1H-indazol-7-yl)pyridine-3-sulfonamide (1-296, 7 mg) as an off-white solid. LCMS (ES, m/z): [M+1]⁺=435; ¹H NMR (400 MHz, DMSO-d₆) δ 4.87 (d, J=8.8 Hz, 1H), 5.62 (d, J=15.4 Hz, 1H), 6.64 (d, J=7.4 Hz, 1H), 7.04 (t, J=7.8 Hz, 1H), 7.78 (d, J=8.0 Hz, 1H), 8.12 (d, J=8.8 Hz, 1H), 8.23-8.12 (m, 1H), 8.28 (dd, J=8.8, 2.4 Hz, 1H), 8.42 (s, 1H), 8.37 (s, 1H), 8.65 (d, J=2.4 Hz, 1H), 9.32 (s, 1H), 10.53 (s, 1H).

### Example 290—Synthesis of N-(6-Methoxy-1-Methyl-1H-Pyrazolo[4,3-C]Pyridin-7-yl)-6-(3-(Trifluoromethyl)-6,7-Dihydropyrano[4,3-C]Pyrazol-1(4H)-yl)Pyridine-3-Sulfonamide (I-300)

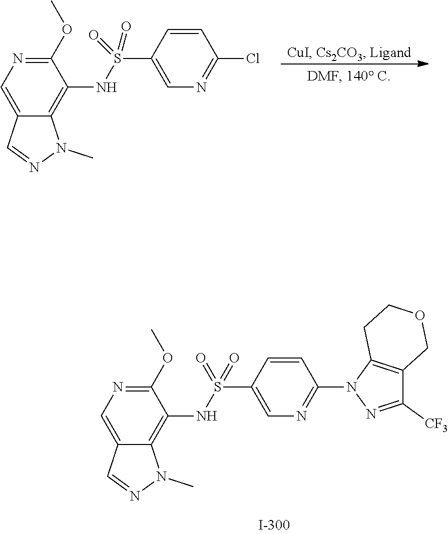 [SMILES: COc1ncc2cnn(C)c2c1NS(=O)(=O)c1ccc(Cl)nc1 ; "CuI, Cs2CO3, Ligand" ; "DMF, 140° C." ; COc1ncc2cnn(C)c2c1NS(=O)(=O)c1ccc(-n2nc(C(F)(F)F)c3c2CCOC3)nc1]

To a stirred solution of 6-chloro-N-(6-methoxy-1-methyl-1H-pyrazolo[4,3-c]pyridin-7-yl)pyridine-3-sulfonamide (100 mg, 0.283 mmol, 1 equiv) and 3-(trifluoromethyl)-1H,4H,6H,7H-pyrano[4,3-c]pyrazole (108.62 mg, 0.566 mmol, 2 equiv) in DMF (3 mL) were added CuI (26.92 mg, 0.141 mmol, 0.5 equiv), (1R,2R)-1-N,2-N-dimethylcyclohexane-1,2-diamine (40.21 mg, 0.283 mmol, 1 equiv), and Cs₂CO₃ (276.29 mg, 0.849 mmol, 3 equiv) at room temperature under a nitrogen atmosphere. The resulting mixture was stirred overnight at 140° C. under a nitrogen atmosphere. The resulting mixture was diluted with water (10 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. This resulted in N-(6-methoxy-1-methyl-1H-pyrazolo[4,3-c]pyridin-7-yl)-6-(3-(trifluoromethyl)-6,7-dihydropyrano[4,3-c]pyrazol-1(4H)-yl)pyridine-3-sulfonamide (1-300, 12.4 mg, 8.58%) as a white solid. LCMS (ES, m/z): [M+1]⁺=510; ¹H NMR (400 MHz, acetonitrile-d₃) δ 3.29 (t, J=5.6 Hz, 2H), 3.34 (s, 3H), 3.91 (t, J=5.6 Hz, 2H), 4.27 (s, 3H), 4.74 (s, 2H), 7.61 (s, 1H), 8.05 (d, J=8.8 Hz, 1H), 8.09 (s, 1H), 8.16 (dd, J=8.8, 2.4 Hz, 1H), 8.58 (s, 1H), 8.64 (d, J=2.4 Hz, 1H).

### Example 291—Synthesis of 6-(6,7-Dihydro-5H-Pyrazolo[5,1-B][1,3]Oxazin-3-yl)-N-(6-Methoxy-1-Methyl-1H-Indazol-7-yl)Pyridine-3-Sulfonamide (I-303)

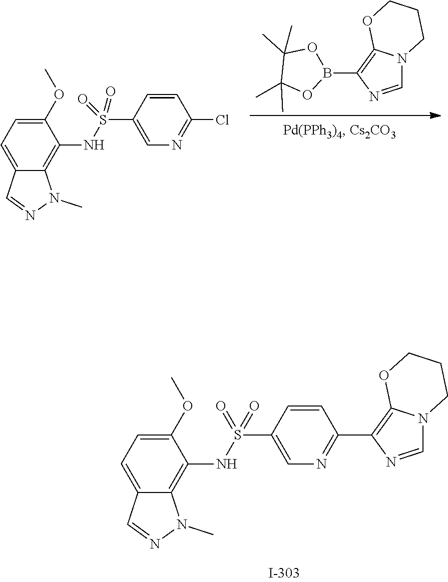

To a stirred solution of 6-chloro-N-(6-methoxy-1-methylindazol-7-yl) pyridine-3-sulfonamide (105 mg, 0.298 mmol, 1 equiv), 3-(4,4,5,5-tetramethyl-1,3,2-dioxaborolan-2-yl)-5H,6H,7H-pyrazolo[3,2-b][1,3]oxazine (82 mg, 0.328 mmol, 1.10 equiv) and Cs₂CO₃ (290 mg, 0.890 mmol, 2.99 equiv) in dioxane (16 mL) and H₂O (4 mL) was added Pd(PPh₃)₄(35 mg, 0.030 mmol, 0.10 equiv) in portions at room temperature under a nitrogen atmosphere. The resulting mixture was stirred for 2 h at 100° C. under a nitrogen atmosphere. The resulting mixture was filtered and the filter cake was washed with MeCN (3×10 mL). The filtrate was concentrated under reduced pressure. The residue was purified by reversed-phase flash chromatography with the following conditions: column: C18 silica gel; mobile phase: MeCN in water (0.1% FA), 30% to 35% gradient in 25 min; detector: UV 220 nm. The crude product (50 mg) was purified by Prep-HPLC with the following conditions (column: Xselect CSH C18 OBD column 30*150 mm 5 μm; mobile phase A: water (0.1% FA), mobile phase B: ACN; flow rate: 60 mL/min; gradient: 19% B to 40% B in 7 min; wavelength: 254 nm/220 nm; RT1(min): 7.7; number of runs: 2) to afford 6-(6,7-dihydro-5H-pyrazolo[5,1-b][1,3]oxazin-3-yl)-N-(6-methoxy-1-methyl-1H-indazol-7-yl)pyridine-3-sulfonamide (I-303, 31.1 mg, 23.49%) as a white solid. LCMS (ES, m/z): [M+1]⁺=441; ¹H NMR (400 MHz, CDCl₃) δ 2.41-2.30 (m, 2H), 3.24 (s, 3H), 4.25 (t, J=6.0 Hz, 2H), 4.42 (s, 3H), 4.46-4.52 (m, 2H), 6.50 (s, 1H), 6.58 (d, J=8.8 Hz, 1H), 7.54 (dd, J=8.4, 0.8 Hz, 1H), 7.58 (d, J=8.8 Hz, 1H), 7.66 (dd, J=8.4, 2.4 Hz, 1H), 7.92 (s, 1H), 8.03 (s, 1H), 8.71 (dd, J=2.4, 0.8 Hz, 1H).

### Example 292—Synthesis of N-(6-Methoxy-1-Methyl-1H-Indazol-7-yl)-6-(3-(Trifluoromethyl)-6,7-Dihydropyrano[4,3-C]Pyrazol-1(4H)-yl)Pyridine-3-Sulfonamide (I-304)

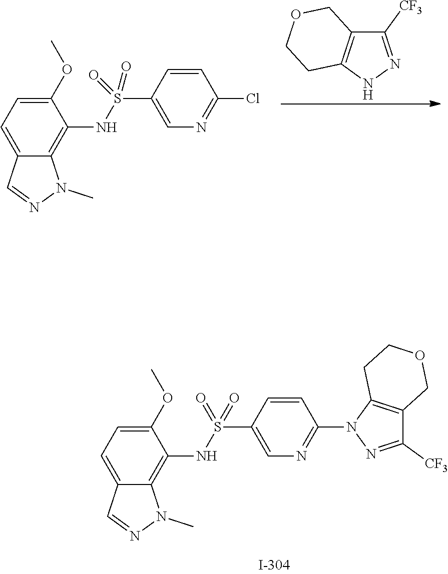 [SMILES: COc1ccc2cnn(C)c2c1NS(=O)(=O)c1ccc(Cl)nc1 ; FC(F)(F)c1n[nH]c2c1COCC2 ; COc1ccc2cnn(C)c2c1NS(=O)(=O)c1ccc(-n2nc(C(F)(F)F)c3c2CCOC3)nc1]

To a stirred mixture of 6-chloro-N-(6-methoxy-1-methylindazol-7-yl)pyridine-3-sulfonamide (100 mg, 0.283 mmol, 1 equiv) and 3-(trifluoromethyl)-1H,4H,6H,7H-pyrano[4,3-c]pyrazole (163.39 mg, 0.849 mmol, 3 equiv) in DMF (2 mL) were added Cs₂CO₃ (277.06 mg, 0.849 mmol, 3 equiv) in portions at 120° C. under a nitrogen atmosphere. The resulting mixture was stirred for an additional 2 days at 120° C. The residue was purified by reversed-phase flash chromatography with the following conditions: column: C18 silica gel; mobile phase: MeCN in water (10 mmol/L NH₄HCO₃), 20% to 45% gradient in 10 min; detector: UV 254 nm. This resulted in N-(6-methoxy-1-methyl-1H-indazol-7-yl)-6-(3-(trifluoromethyl)-6,7-dihydropyrano[4,3-c]pyrazol-1(4H)-yl)pyridine-3-sulfonamide (1-304, 68.7 mg, 47.33%) as a light brown solid. LCMS (ES, m/z): [M+1]⁺=509; ¹H NMR (400 MHz, chloroform-d) δ3.28 (s, 3H), 3.33 (dd, J=6.4, 4.8 Hz, 2H), 3.96 (t, J=5.6 Hz, 2H), 4.42 (s, 3H), 4.78 (s, 2H), 6.52 (s, 1H), 6.60 (d, J=8.8 Hz, 1H), 7.63 (d, J=8.8 Hz, 1H), 7.96 (d, J=9.0 Hz, 2H), 8.04 (dd, J=8.6, 0.8 Hz, 1H), 8.61 (dd, J=2.4, 0.8 Hz, 1H).

### Example 293—Synthesis of 6-(5,6-Dihydro-8H-Imidazo[2,1-C][1,4]Oxazin-3-yl)-N-(1-Methyl-1H-Indazol-7-yl)Pyridine-3-Sulfonamide (I-309)

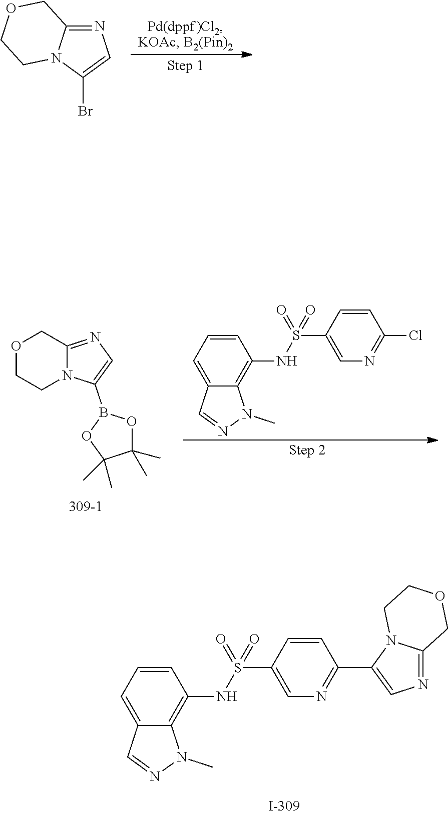

Step 1: Synthesis of 309-1: To a stirred mixture of 3-bromo-5H,6H,8H-imidazo[2,1-c][1,4]oxazine (200 mg, 0.985 mmol, 1 equiv) and Pd(dppf)Cl₂ (360.38 mg, 0.492 mmol, 0.5 equiv) in 1,4-dioxane (10 mL) were added KOAc (290.02 mg, 2.955 mmol, 3 equiv) and 4,4,5,5-tetramethyl-2-(4,4,5,5-tetramethyl-1,3,2-dioxaborolan-2-yl)-1,3,2-dioxaborolane (309-1, 500.28 mg, 1.970 mmol, 2 equiv) in portions at 100° C. under a nitrogen atmosphere. The resulting mixture was stirred overnight at 100° C. The crude product was used in the next step directly without further purification.

Step 2: Synthesis of 6-(5,6-dihydro-8H-imidazo[2,1-c][1,4]oxazin-3-yl)-N-(1-methyl-1H-indazol-7-yl)pyridine-3-sulfonamide (I-309): To a stirred mixture of 309-1 (100 mg, 0.400 mmol, 1 equiv) and 6-chloro-N-(1-methylindazol-7-yl)pyridine-3-sulfonamide (258.10 mg, 0.800 mmol, 2 equiv) in DMSO (10 mL) was added Cs₂CO₃ (390.81 mg, 1.200 mmol, 3 equiv) in portions at 100° C. under a nitrogen atmosphere. The resulting mixture was stirred for an additional 3 h at 100° C. The residue was purified by reversed-phase flash chromatography with the following conditions: column: C18 silica gel; mobile phase: MeCN in water (0.1% FA), 10% to 30% gradient in 10 min; detector: UV 254 nm. The crude product (100 mg) was purified by Prep-HPLC with the following conditions (column: Xselect CSH C18 OBD column 30*150 mm 5 μm; mobile phase A: water (0.1% FA), mobile phase B: ACN; flow rate: 60 mL/min; gradient: 11% B to 25% B in 7 min; wavelength: 254 nm/220 nm; RT1(min): 6.5) to afford 6-(5,6-dihydro-8H-imidazo[2,1-c][1,4]oxazin-3-yl)-N-(1-methyl-1H-indazol-7-yl)pyridine-3-sulfonamide (I-309, 55.3 mg, 33.39%) as a white solid. LCMS (ES, m/z): [M+1]⁺=411; ¹H NMR (400 MHz, chloroform-d) δ4.09 (t, J=5.2 Hz, 2H), 4.41 (s, 3H), 4.58 (t, J=5.2 Hz, 2H), 4.95 (s, 2H), 6.49 (s, 1H), 6.57 (d, J=7.4 Hz, 1H), 6.88-6.96 (m, 1H), 7.65 (d, J=7.8 Hz, 3H), 7.71 (dd, J=8.2, 1.0 Hz, 1H), 7.88 (dd, J=8.6, 2.4 Hz, 1H), 8.79 (dd, J=2.6, 0.8 Hz, 1H).

### Example 294—Synthesis of 6-(7,8-Dihydro-4H,6H-Pyrazolo[5,1-C][1,4]Oxazepin-3-yl)-N-(6-Methoxy-1-Methyl-1H-Indazol-7-yl)Pyridine-3-Sulfonamide (I-310)

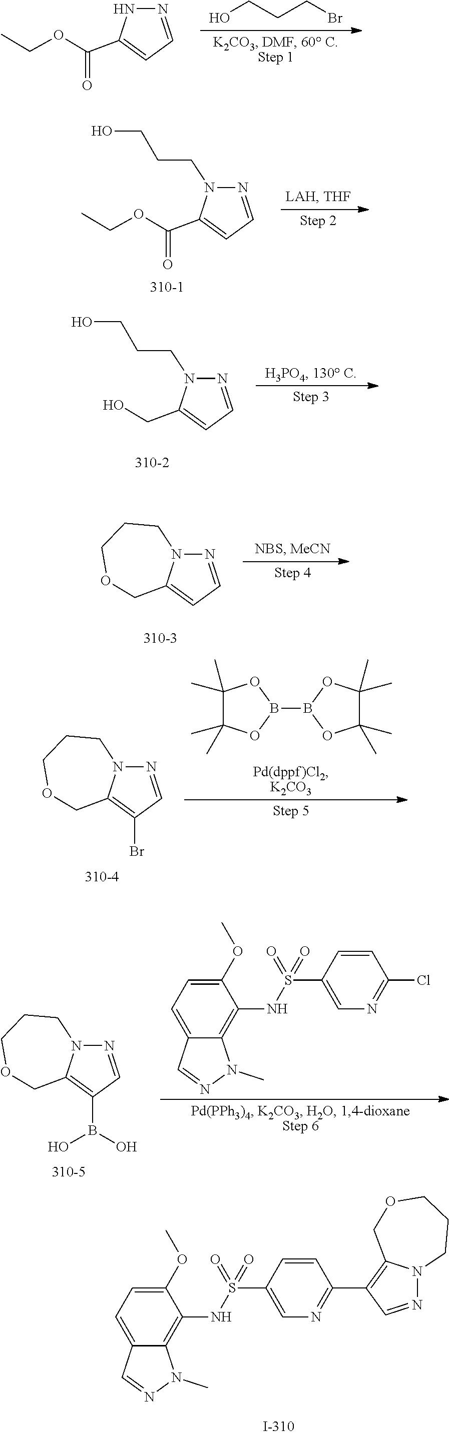

Step 1: Synthesis of 310-1: A solution of ethyl 2H-pyrazole-3-carboxylate (10 g, 71.35 mmol, 1 equiv), 3-bromopropanol (18.84 g, 142.7 mmol, 2 equiv) and K₂CO₃ (29.585 g, 214.1 mmol, 3 equiv) in DMF (150 mL) was stirred overnight at 60° C. After the reaction was complete, the resulting mixture was diluted with water (1000 mL). The resulting mixture was extracted with EtOAc (4×400 mL). The combined organic layers were washed with brine (1×500 mL) and dried over anhydrous Na₂SO₄. After filtration, the filtrate was concentrated under reduced pressure to afford the crude. The residue was purified by reversed-phase flash chromatography with the following conditions: column: C18 silica gel; mobile phase: ACN in water, 0% to 100% gradient in 40 min; detector: UV 254 nm to afford 310-1 (4.15 g, 29.34%) as a colorless oil.

Step 2: Synthesis of 310-2: To a stirred solution of 310-1 (3.5 g, 17.65 mmol, 1 equiv) in THF (140 mL) was added LiAlH₄ (957.3 mg, 20.2 mmol, 2 equiv) in portions at 0° C. under a nitrogen atmosphere. The resulting mixture was stirred for 1 h at 0° C. under a nitrogen atmosphere. After the reaction was complete, the reaction was quenched with sodium sulfate decahydrate at room temperature. Then the mixture was stirred for 30 min. After filtration, the solution was concentrated under vacuum to give 310-2 (2.3 g, 76.15%) as a colorless oil.

Step 3: Synthesis of 310-3: A solution of 310-2 (2.5 g, 16.01 mmol, 1 equiv) in H₃PO₄ (70 mL) was stirred for 20 h at 130° C. After the reaction was complete, The reaction was quenched with water (60 mL). The mixture was basified to pH 8 with saturated NaHCO₃ (aq.). The aqueous layer was extracted with EtOAc (4×200 mL). The combined organic layers were washed with brine (300 mL) and dried over anhydrous Na₂SO₄. After filtration, the filtrate was concentrated under reduced pressure to afford 310-3 (1.031 g, 46.63%) as a light yellow oil.

Step 4: Synthesis of 310-4: To a stirred solution 310-3 (980 mg, 7.093 mmol, 1 equiv) in ACN (13 mL) was added NBS (1.51 g, 8.512 mmol, 1.2 equiv) at 0° C. The reaction was then stirred at room temperature for 16 h. The reaction was quenched with water at room temperature. The resulting mixture was extracted with CH₂Cl₂ (4×20 mL). The combined organic layers were washed with brine (50 mL) and dried over anhydrous Na₂SO₄. After filtration, the filtrate was purified by reversed-phase flash chromatography with the following conditions: column: C18 silica gel; mobile phase: ACN in water (0.1% FA), 0% to 100% gradient in 10 min; detector: UV 254 nm to afford 310-4 (698 mg, 45.34%) as a light pink solid.

Step 5: Synthesis of 310-5: A solution of 310-4 (152.5 mg, 0.703 mmol, 1 equiv), bis(pinacolato)diboron (892.03 mg, 3.515 mmol, 5 equiv), Pd(dppf)Cl₂ (77.11 mg, 0.105 mmol, 0.15 equiv) and K₂CO₃ (194.19 mg, 1.406 mmol, 2 equiv) in dioxane (7 mL) was stirred overnight at 80° C. under a nitrogen atmosphere. The resulting mixture was concentrated under vacuum. The crude product was purified by reversed-phase flash chromatography with the following conditions: column: C18 silica gel; mobile phase: ACN in water (10 mmol/L NH₄HCO₃), 0% to 100% gradient in 10 min; detector: UV 254 nm to afford 310-5 (100 mg, 53.89%) as a white solid.

Step 6: Synthesis of 6-(7,8-dihydro-4H,6H-pyrazolo[5,1-c][1,4]oxazepin-3-yl)-N-(6-methoxy-1-methyl-1H-indazol-7-yl)pyridine-3-sulfonamide (1-310): A solution of 310-5 (60 mg, 0.227 mmol, 1 equiv), 6-chloro-N-(6-methoxy-1-methylindazol-7-yl)pyridine-3-sulfonamide (88.15 mg, 0.250 mmol, 1.1 equiv), Pd(dppf)Cl₂ (16.62 mg, 0.023 mmol, 0.1 equiv) and K₂CO₃ (62.79 mg, 0.454 mmol, 2 equiv) in dioxane (10 mL) and H₂O (2 mL) was stirred overnight at 100° C. under a nitrogen atmosphere. After the reaction was completed, the solvent was concentrated under vacuum to give a residue. The residue was purified by reversed-phase flash chromatography with the following conditions: column: C18 silica gel; mobile phase: ACN in water (0.1% FA), 0% to 100% gradient in 30 min; detector: UV 254 nm to give the crude. The crude was further purified by Prep-HPLC with the following conditions (column: RP Prep OBD C18 column, 30*150 mm, 5 μm; mobile phase A: water (10 mmol/L NH₄HCO₃+0.05% NH₃H₂O), mobile phase B: ACN; flow rate: 60 mL/min; gradient: 8% B to 38% B in 8 min; wavelength: 220 nm; RT 1(min): 9.67) to afford 6-(7,8-dihydro-4H,6H-pyrazolo[5,1-c][1,4]oxazepin-3-yl)-N-(6-methoxy-1-methyl-1H-indazol-7-yl)pyridine-3-sulfonamide (I-310, 41.8 mg, 40.49%) as an off-white solid. LCMS (ES, m/z): [M+1]⁺455; ¹H NMR (400 MHz, DMSO-d₆) δ3.13 (s, 3H), 3.99 (t, J=3.6 Hz, 2H), 4.01 (s, 3H) 4.49 (t, J=3.6 Hz, 2H), 5.21 (s, 1H), 6.81 (d, J=8.8 Hz, 1H), 7.66 (d, J=8.8 Hz, 1H), 7.85 (d, J=8.4 Hz, 1H), 7.93-8.00 (m, 2H), 8.68 (d, J=2.0 Hz, 1H), 9.92 (s, 1H).

### Example 295—Synthesis of 6-(5,6-Dihydro-8H-Imidazo[2,1-C][1,4]Oxazin-3-yl)-N-(6-Methoxy-1-Methyl-1H-Indazol-7-yl)Pyridine-3-Sulfonamide (I-312)

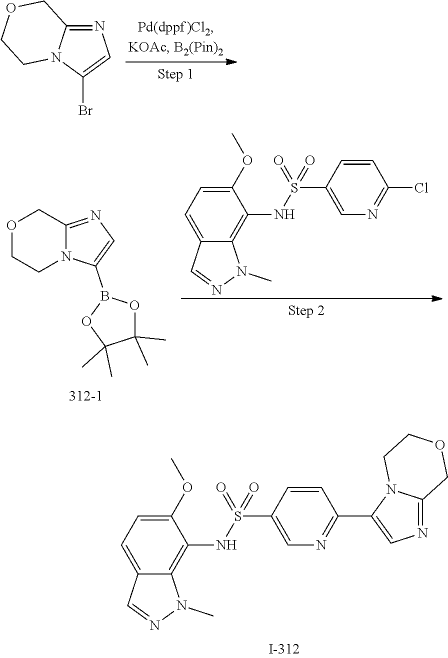 [SMILES: Brc1cnc2n1CCOC2 ; CC1(C)OB(c2cnc3n2CCOC3)OC1(C)C ; COc1ccc2cnn(C)c2c1NS(=O)(=O)c1ccc(Cl)nc1 ; COc1ccc2cnn(C)c2c1NS(=O)(=O)c1ccc(-c2cnc3n2CCOC3)nc1]

Step 1: Synthesis of 312-1: To a stirred mixture of 3-bromo-5,6-dihydro-8H-imidazo[2,1-c][1,4]oxazine (200 mg, 0.985 mmol, 1 equiv) and Pd(dppf)Cl₂ (360.38 mg, 0.492 mmol, 0.5 equiv) in 1,4-dioxane (10 mL) were added KOAc (290.02 mg, 2.955 mmol, 3 equiv) and 4,4,5,5-tetramethyl-2-(4,4,5,5-tetramethyl-1,3,2-dioxaborolan-2-yl)-1,3,2-dioxaborolane (500.28 mg, 1.970 mmol, 2 equiv) in portions at 100° C. under a nitrogen atmosphere. The resulting mixture was stirred overnight at 100° C. The crude product was used in the next step directly without further purification. This resulted in 3-(4,4,5,5-tetramethyl-1,3,2-dioxaborolan-2-yl)-5,6-dihydro-8H-imidazo[2,1-c] [1,4]oxazine (312-1) as a black solid.

Step 2: Synthesis of 6-(5,6-dihydro-8H-imidazo[2,1-c][1,4]oxazin-3-yl)-N-(6-methoxy-1-methyl-1H-indazol-7-yl)pyridine-3-sulfonamide (I-312): A mixture of 3-(4,4,5,5-tetramethyl-1,3,2-dioxaborolan-2-yl)-5,6-dihydro-8H-imidazo[2,1-c] [1,4]oxazine (100 mg, 0.400 mmol, 1 equiv), N-(6-methoxy-1-methylindazol-7-yl)pyridine-3-sulfonamide (127.28 mg, 0.400 mmol, 1 equiv) and Cs₂CO₃ (390.81 mg, 1.200 mmol, 3 equiv) in DMSO (1 mL) was stirred for 3 h at 100° C. under a nitrogen atmosphere. The residue was purified by reversed-phase flash chromatography with the following conditions: column: XBridge Shield RP18 OBD column, 30*150 mm, 5 μm; mobile phase A: water (10 mmol/L NH₄HCO₃+0.05% NH₃H₂O), mobile phase B: ACN; flow rate: 60 mL/min; gradient: 13% B to 34% B in 7 min; wavelength: 254 nm/220 nm; RT1(min): 7; number of runs: 4. This resulted in 6-(5,6-dihydro-8H-imidazo[2,1-c][1,4]oxazin-3-yl)-N-(6-methoxy-1-methyl-1H-indazol-7-yl)pyridine-3-sulfonamide (I-312, 4.2 mg, 2.36%) as an off-white solid. LCMS (ES, m/z): [M+1]⁺=441; ¹H NMR (400 MHz, DMSO-d₆) δ 3.17 (s, 3H), 4.05 (t, J=5.2 Hz, 2H), 4.27 (s, 3H), 4.46 (t, J=5.2 Hz, 2H), 4.84 (s, 2H), 6.82 (d, J=8.9 Hz, 1H), 7.66 (d, J=8.8 Hz, 1H), 7.78 (s, 1H), 8.01-7.92 (m, 3H), 8.67 (t, J=1.7 Hz, 1H), 9.92 (s, 1H).

### Example 296—Synthesis of N-(1-Methyl-1H-Indazol-7-yl)-6-(5-Methyl-4,5,6,7-Tetrahydropyrazolo[1,5-A]Pyrazin-3-yl)Pyridine-3-Sulfonamide (I-314)

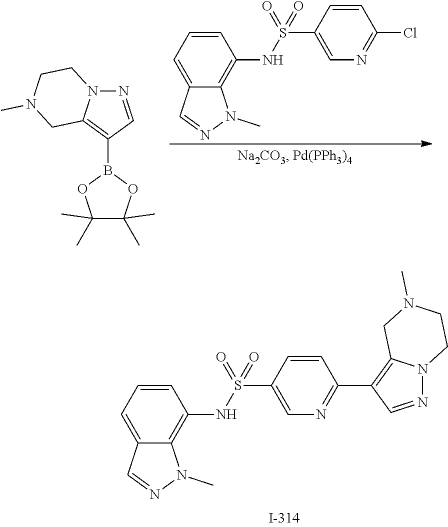

A mixture of 6-chloro-N-(1-methylindazol-7-yl)pyridine-3-sulfonamide (100 mg, 0.310 mmol, 1 equiv), 5-methyl-3-(4,4,5,5-tetramethyl-1,3,2-dioxaborolan-2-yl)-4H,6H,7H-pyrazolo[1,5-a]pyrazine (97.83 mg, 0.372 mmol, 1.2 equiv), Pd(PPh₃)₄(35.80 mg, 0.031 mmol, 0.1 equiv), and Na₂CO₃ (98.51 mg, 0.930 mmol, 3 equiv) in dioxane (4 mL) and H₂O (1 mL) was stirred overnight at 80° C. under a nitrogen atmosphere. The resulting mixture was filtered and the filter cake was washed with EtOH (2×6 mL). The filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 6-{5-methyl-4H,6H,7H-pyrazolo[1,5-a]pyrazin-3-yl}-N-(1-methylindazol-7-yl)pyridine-3-sulfonamide (I-314, 76.8 mg, 58.53%) as a yellow solid. LCMS (ES, m/z): [M+H]⁺=424; ¹H NMR (400 MHz, DMSO-d₆): δ 1.99 (m, J=12.4, 4.0 Hz, 2H), 2.35 (m, 2H), 2.54-2.64 (m, 2H), 3.53 (d, J=12.0 Hz, 3H), 3.80 (s, 3H), 3.98 (s, 3H), 4.30 (s, 3H), 7.05 (d, J=8.8 Hz, 1H), 7.27 (dd, J=12.6, 1.0 Hz, 2H), 7.71 (d, J=8.8 Hz, 1H), 7.91 (s, 1H).

### Example 297—Synthesis of N-(6-(Methoxy-D3)-1-(Methyl-D3)-1H-Indazol-7-yl)-6-(4-(Trifluoromethyl)-1H-Pyrazol-1-yl)Pyridine-3-Sulfonamide (I-316)

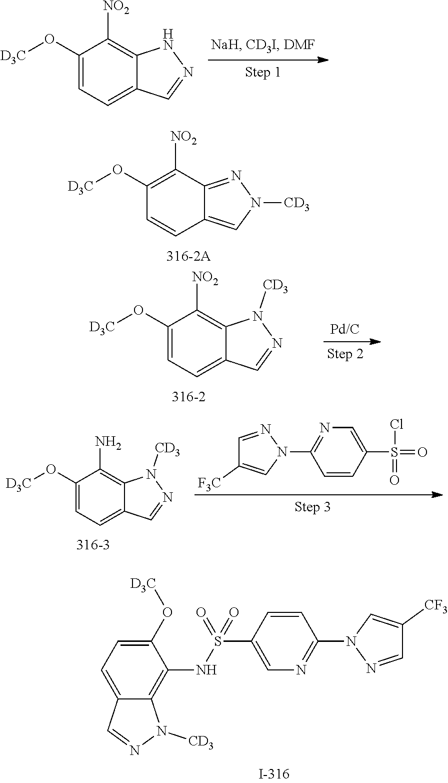

Step 1: Synthesis of 316-2: To a stirred solution of 6-(2H3)methoxy-7-nitro-1H-indazole (3 g, 15.292 mmol, 1 equiv) and CD₃I (3.02 mL, 45.876 mmol, 3 equiv) in DMF (30 mL) was added NaH (1.10 g, 45.876 mmol, 3 equiv) at room temperature under a nitrogen atmosphere. The resulting mixture was stirred for 2 h at room temperature under a nitrogen atmosphere. The residue was purified by silica gel column chromatography and eluted with PE/EA (7:1) to afford 6-(2H3)methoxy-1-(2H3)methyl-7-nitroindazole (316-2, 1.5 g, 41.40%) as a white solid

Step 2: Synthesis of 316-3: A solution of 6-(2H3)methoxy-1-(2H3)methyl-7-nitroindazole (1.5 g, 7.035 mmol, 1 equiv) and Pd/C (2.25 g, 21.105 mmol, 3 equiv) in MeOH (15 mL) was stirred for 3 h at room temperature under a nitrogen atmosphere. The resulting mixture was concentrated under vacuum. This resulted in 6-(2H3)methoxy-1-(2H3)methylindazol-7-amine (316-3, 1.1 g, 76.80%) as a yellow oil.

Step 3: Synthesis of N-(6-(methoxy-d3)-1-(methyl-d3)-1H-indazol-7-yl)-6-(4-(trifluoromethyl)-1H-pyrazol-1-yl)pyridine-3-sulfonamide (I-316): A solution of 6-(2H3)methoxy-1-(2H3)methylindazol-7-amine (300 mg, 1.637 mmol, 1 equiv) and 6-[4-(trifluoromethyl)pyrazol-1-yl]pyridine-3-sulfonyl chloride (510.24 mg, 1.637 mmol, 1 equiv) in pyridine (1 mL) was stirred at room temperature under a nitrogen atmosphere. The resulting mixture was stirred for 2 h at room temperature under a nitrogen atmosphere. The resulting mixture was concentrated under vacuum. The residue was purified by reversed-phase flash chromatography with the following conditions: column: C18 silica gel; mobile phase: MeCN in water (0.1% FA), 0% to 100% gradient in 20 min; detector: UV 254 nm. This resulted in N-[6-(2H3)methoxy-1-(2H3)methylindazol-7-yl]-6-[4-(trifluoromethyl)pyrazol-1-yl]pyridine-3-sulfonamide (I-316, 93.3 mg, 12.39%) as a white solid. LCMS (ES, m/z): [M+H]⁺=459; ¹H NMR (400 MHz, DMSO-d₆) δ 6.82 (d, J=8.8 Hz, 1H), 7.68 (d, J=8.8 Hz, 1H), 7.99 (s, 1H), 8.17 (d, J=8.7 Hz, 1H), 8.28 (dd, J=2.4, 8.7 Hz, 1H), 8.40 (s, 1H), 8.68 (dd, J=0.8, 2.4 Hz, 1H), 9.32 (t, J=1.1 Hz, 1H), 10.11 (s, 1H).

### Example 298—Synthesis of 6-(3-Isopropyl-1H-Pyrazol-1-yl)-N-Methyl-N-(1-Methyl-1H-Indazol-7-yl)Pyridine-3-Sulfonamide (I-171)

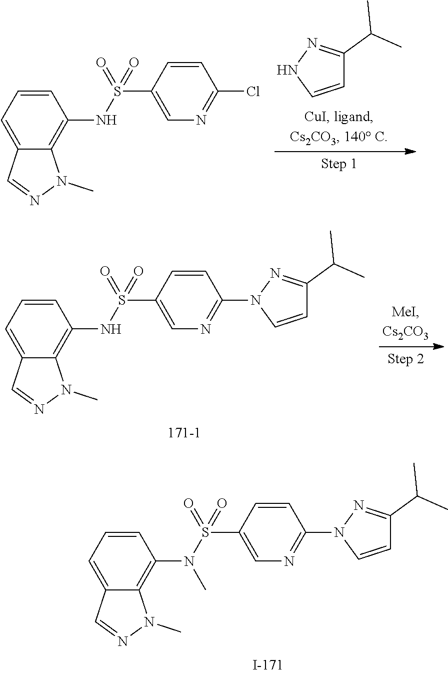

Step 1: Synthesis of 171-1: To a stirred mixture of 6-chloro-N-(1-methylindazol-7-yl)pyridine-3-sulfonamide (500 mg, 1.549 mmol, 1 equiv) and 3-isopropyl-1H-pyrazole (170.65 mg, 1.549 mmol, 1 equiv) in DMF (10 mL) were added CuI (147.51 mg, 0.774 mmol, 0.5 equiv) and Cs₂CO₃ (1514.17 mg, 4.647 mmol, 3 equiv) and N1,N2-dimethylcyclohexane-1,2-diamine (220.35 mg, 1.549 mmol, 1 equiv) at room temperature under a nitrogen atmosphere. The resulting mixture was stirred overnight at 140° C. under a nitrogen atmosphere. The resulting mixture was diluted with water (10 mL). The resulting mixture was extracted with EtOAc (2×8 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 0% to 100% gradient in 30 min; detector: UV 254 nm. This resulted in 6-(3-isopropylpyrazol-1-yl)-N-(1-methylindazol-7-yl)pyridine-3-sulfonamide (171-1, 60 mg, 8.79%) as an off-white solid.

Step 2: Synthesis of 6-(3-isopropyl-1H-pyrazol-1-yl)-N-methyl-N-(1-methyl-1H-indazol-7-yl)pyridine-3-sulfonamide (I-171): To a stirred mixture of 6-(3-isopropylpyrazol-1-yl)-N-(1-methylindazol-7-yl)pyridine-3-sulfonamide (40 mg, 0.101 mmol, 1 equiv) and CH₃I (14.32 mg, 0.101 mmol, 1 equiv) in DMF (1 mL) was added Cs₂CO₃ (98.62 mg, 0.303 mmol, 3 equiv) at room temperature under a nitrogen atmosphere. The resulting mixture was stirred for 1 h at room temperature under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The resulting mixture was concentrated under reduced pressure. The residue was purified by reversed-phase flash chromatography with the following conditions: column: C18 silica gel; mobile phase: MeCN in water, 0% to 100% gradient in 20 min; detector: UV 254 nm. The resulting mixture was concentrated under reduced pressure. This resulted in 6-(3-isopropylpyrazol-1-yl)-N-methyl-N-(1-methylindazol-7-yl)pyridine-3-sulfonamide (I-171, 22.3 mg, 53.47%) as an off-white solid. LCMS [M+1]⁺=411; ¹H NMR (400 MHz, DMSO-d₆) δ 1.29 (3, 6H), 3.02-3.06 (m, 1H), 3.32 (s, 3H), 4.31 (s, 3H), 6.58 (d, J=2.8 Hz, 1H), 6.77 (d, J=7.2 Hz, 1H), 7.02 (t, J=7.6 Hz, 1H), 7.79 (d, J=8.0 Hz, 1H), 8.07 (d, J=8.4 Hz, 1H), 8.13 (d, J=4.4 Hz, 1H), 8.14 (d, J=4.4 Hz, 1H), 8.61 (s, 1H), 8.62 (s, 1H).

### Example 299—Synthesis of 6-(4-Isopropyl-1H-Pyrazol-1-yl)-N-(6-Methoxy-1-Methyl-1H-Indazol-7-yl)-N-Methylpyridine-3-Sulfonamide (I-172)

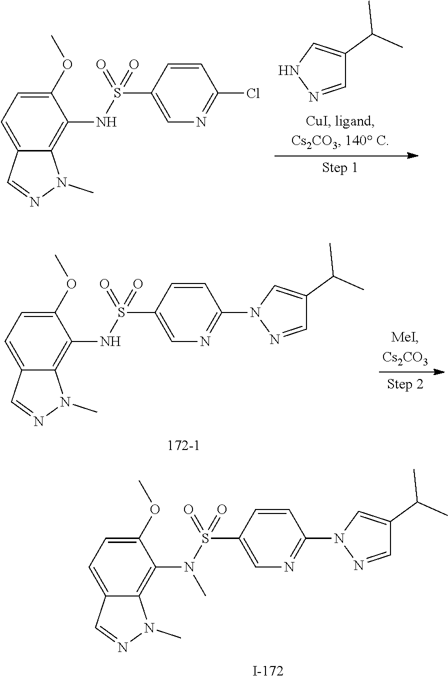

Step 1: Synthesis of 172-1: To a stirred mixture of 6-chloro-N-(6-methoxy-1-methylindazol-7-yl)pyridine-3-sulfonamide (500 mg, 1.417 mmol, 1 equiv) and 4-isopropyl-1H-pyrazole (156.13 mg, 1.417 mmol, 1 equiv) in DMF (5 mL) were added CuI (134.96 mg, 0.709 mmol, 0.5 equiv), Cs₂CO₃ (1385.32 mg, 4.251 mmol, 3 equiv), and N1,N2-dimethylcyclohexane-1,2-diamine (201.60 mg, 1.417 mmol, 1 equiv) in portions at room temperature under a nitrogen atmosphere. The resulting mixture was stirred overnight at 140° C. under a nitrogen atmosphere. The resulting mixture was diluted with water (10 mL). The resulting mixture was extracted with EtOAc (3×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeOH in water, 0% to 100% gradient in 30 min; detector: UV 254 nm. This resulted in 6-(4-isopropylpyrazol-1-yl)-N-(6-methoxy-1-methylindazol-7-yl)pyridine-3-sulfonamide (172-1, 60 mg, 8.93%) as an off-white solid.

Step 2: Synthesis of 6-(4-isopropyl-1H-pyrazol-1-yl)-N-(6-methoxy-1-methyl-11H-indazol-7-yl)-N-methylpyridine-3-sulfonamide (I-172): To a stirred mixture of 6-(4-isopropylpyrazol-1-yl)-N-(6-methoxy-1-methylindazol-7-yl) pyridine-3-sulfonamide (50 mg, 0.117 mmol, 1 equiv) and CH₃I (16.64 mg, 0.117 mmol, 1 equiv) in DMF (1 mL) was added Cs₂CO₃ (114.59 mg, 0.351 mmol, 3 equiv) at room temperature under a nitrogen atmosphere. The resulting mixture was stirred for 1 h at room temperature under a nitrogen atmosphere. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The resulting mixture was concentrated under reduced pressure. The residue was purified by reversed-phase flash chromatography with the following conditions: column: C18 silica gel; mobile phase: MeCN in water, 0% to 100% gradient in 20 min; detector: UV 254 nm. The resulting mixture was concentrated under vacuum. This resulted in 6-(4-isopropylpyrazol-1-yl)-N-(6-methoxy-1-methylindazol-7-yl)-N-methylpyridine-3-sulfonamide (I-172, 40.3 mg, 77.49%) as an off-white solid. LCMS (ES, m/z): [M+1]⁺=441; ¹H NMR (400 MHz, DMSO-d₆) δ 1.25 (d, J=6.9 Hz, 6H), 2.89-2.91 (m, J=6.7 Hz, 1H), 3.27 (s, 3H), 3.32 (s, 3H), 4.23 (s, 3H), 6.91 (d, J=8.9 Hz, 1H), 7.76 (d, J=8.8 Hz, 1H), 7.88 (s, 1H), 8.02 (s, 1H), 8.09 (d, J=8.7 Hz, 1H), 8.22 (dd, J=2.4, 8.7 Hz, 1H), 8.48 (s, 1H), 8.66 (d, J=2.4 Hz, 1H).

### Example 300—Synthesis of N-(6-Methoxy-1-Methyl-1H-Indazol-7-yl)-6-(2-Methylthiazol-4-yl)Pyridine-3-Sulfonamide (I-173)

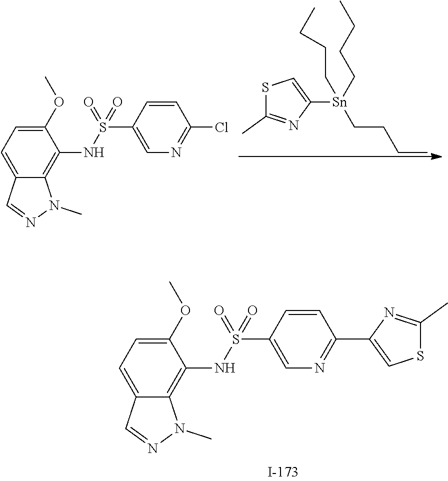

A mixture of 6-chloro-N-(6-methoxy-1-methylindazol-7-yl)pyridine-3-sulfonamide (60 mg, 0.170 mmol, 1 equiv), XPhos Pd G3 (14.40 mg, 0.017 mmol, 0.1 equiv), XPhos (16.22 mg, 0.034 mmol, 0.2 equiv), ZnCl₂ (69.53 mg, 0.510 mmol, 3 equiv) and 2-methyl-4-(tributylstannyl)-1,3-thiazole (79.23 mg, 0.204 mmol, 1.2 equiv) in DMF (2 mL) was stirred for 2 h at 90° C. under a nitrogen atmosphere. The desired product could be detected by LCMS. The reaction was quenched with water (50 mL) at room temperature. The resulting mixture was extracted with EtOAc (3×200 mL). The combined organic layers were dried over anhydrous MgSO₄. After filtration, the filtrate was concentrated under reduced pressure. The crude product (250 mg) was purified by Prep-HPLC with the following conditions (column: XBridge Prep OBD C18 column: 30*150 mm, 5 m; mobile phase A: water (10 mmol/L NH₄HCO₃+0.05% NH₃H₂O), mobile phase B: ACN; flow rate: 60 mL/min; gradient: 21% B to 46% B in 7 min; wavelength: 220 nm; RT1(min): 7.92; number ff runs: 4) to afford N-(6-methoxy-1-methylindazol-7-yl)-6-(2-methyl-1,3-thiazol-4-yl)pyridine-3-sulfonamide (I-173, 21.4 mg, 29.65%) as a white solid. LCMS (ES, m/z): [M+H]⁺=416; H-NMR: (400 MHz, DMSO, ppm) δ 2.76 (s, 3H), 3.11 (s, 3H), 4.27 (s, 3H), 6.81 (d, J=9.2 Hz, 1H), 7.66 (d, J=8.8 Hz, 1H), 7.99 (s, 1H), 8.09 (dd, J=8.4, 2.4 Hz, 1H), 8.20 (d, J=8.0 Hz, 1H), 8.33 (s, 1H), 8.75 (d, J=2.0 Hz, 1H), 9.95 (s, 1H).

### Example 301—Synthesis of N-(5-Methoxy-3-Methyl-1H-Indol-4-yl)-6-(4-(Trifluoromethyl)-1H-Pyrazol-1-yl)Pyridine-3-Sulfonamide (I-174)

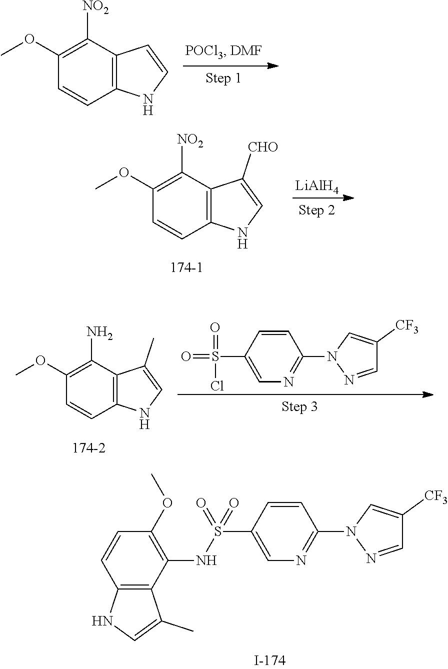

Step 1: Synthesis of 174-1: A solution of 5-methoxy-4-nitro-1H-indole (300 mg, 1.561 mmol, 1 equiv) and POCl₃ (287.21 mg, 1.873 mmol, 1.2 equiv) in DMF (5 mL) was stirred for 30 min at 0° C. under a nitrogen atmosphere. The resulting mixture was stirred for 2 h at 40° C. under an air atmosphere, then 2 M NaOH was added slowly, and the mixture was heated to 90° C. for another 1 h. The reaction was quenched with water (10 mL) at 0° C. The aqueous layer was extracted with EtOAc (3×20 mL). The combined organic layers were washed with brine (30 mL) and dried over anhydrous Na₂SO₄. The resulting mixture was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 5-methoxy-4-nitro-1H-indole-3-carbaldehyde (174-1, 240 mg, 64.94%) as a yellow solid.

Step 2: Synthesis of 174-2: To a stirred solution of 5-methoxy-4-nitro-1H-indole-3-carbaldehyde (100 mg, 0.454 mmol, 1 equiv) in THE (2 mL) was added LiAlH₄ (86.18 mg, 2.270 mmol, 5 equiv) at 0° C. under a nitrogen atmosphere. The resulting mixture was stirred for 2 h at 50° C. under an air atmosphere. The reaction was quenched with water (20 mL) at 0° C. The aqueous layer was extracted with EtOAc (3×20 mL). The combined organic layers were washed with brine (30 mL), dried over anhydrous Na₂SO₄ and concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in 5-methoxy-3-methyl-1H-indol-4-amine (174-2, 70 mg, 52.48%) as a purple solid.

Step 3: Synthesis of N-(5-methoxy-3-methyl-1H-indol-4-yl)-6-(4-(trifluoromethyl)-1H-pyrazol-1-yl)pyridine-3-sulfonamide (I-174): A solution of 5-methoxy-3-methyl-1H-indol-4-amine (50 mg, 0.284 mmol, 1 equiv) and 6-[4-(trifluoromethyl)pyrazol-1-yl]pyridine-3-sulfonyl chloride (88.43 mg, 0.284 mmol, 1 equiv) in pyridine (1 mL) was stirred for 16 h at room temperature under an air atmosphere. The resulting mixture was concentrated under reduced pressure. The resulting mixture was diluted with water (10 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in N-(5-methoxy-3-methyl-1H-indol-4-yl)-6-[4-(trifluoromethyl)pyrazol-1-yl]pyridine-3-sulfonamide (I-174, 3.6 mg, 2.74%) as a white solid. LCMS (ES, m/z): [M+H]⁺=452; ¹H NMR (400 MHz, DMSO) δ 2.48 (s, 3H), 3.10 (d, J=1.2 Hz, 3H), 6.69 (d, J=8.8 Hz, 1H), 7.10 (s, 1H), 7.23 (dd, J=8.8, 1.2 Hz, 1H), 8.16 (d, J=8.8 Hz, 1H), 8.27 (dt, J=8.8, 1.6 Hz, 1H), 8.40 (s, 1H), 8.67 (d, J=2.4 Hz, 1H), 9.32 (s, 1H), 9.72 (s, 1H), 10.69 (s, 1H).

### Example 302—Synthesis of N-(6-Methoxy-1H-Indazol-7-yl)-6-(4-(Trifluoromethyl)-1H-Pyrazol-1-yl)Pyridine-3-Sulfonamide (I-175)

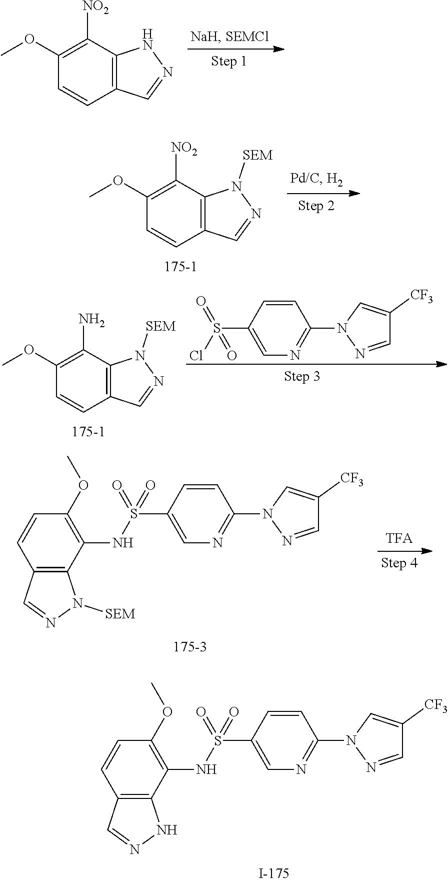

Step 1: Synthesis of 175-1: To a stirred solution of 6-methoxy-7-nitro-1H-indazole (500 mg, 2.589 mmol, 1 equiv) and t-BuOK (726.16 mg, 6.473 mmol, 2.5 equiv) in THE (10 mL) was added SEM-Cl (647.33 mg, 3.883 mmol, 1.5 equiv) dropwise at 0° C. under a nitrogen atmosphere. The resulting mixture was stirred for 2 h at room temperature under a nitrogen atmosphere. The reaction was quenched with water (20 mL) at 0° C. The aqueous layer was extracted with EtOAc (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in 6-methoxy-7-nitro-1-{[2-(trimethylsilyl)ethoxy]methyl}indazole (175-1, 122 mg, 14.57%) as a yellow solid.

Step 2: Synthesis of 175-2: A solution of 6-methoxy-7-nitro-1-{[2-(trimethylsilyl)ethoxy]methyl}indazole (200 mg, 0.618 mmol, 1 equiv) and Pd/C (329.04 mg, 3.090 mmol, 5 equiv) in CH₃OH (20 mL) was stirred for 2 h at room temperature under a hydrogen atmosphere. The resulting mixture was filtered and the filter cake was washed with DCM (3×10 mL). The filtrate was concentrated under reduced pressure. This resulted in 6-methoxy-1-{[2-(trimethylsilyl)ethoxy]methyl}indazol-7-amine (175-2, 123 mg, 54.23%) as a purple oil.

Step 3: Synthesis of 175-3: A solution of 6-methoxy-1-{[2-(trimethylsilyl)ethoxy]methyl}indazol-7-amine (68 mg, 0.232 mmol, 1 equiv) and 6-[4-(trifluoromethyl)pyrazol-1-yl]pyridine-3-sulfonyl chloride (72.22 mg, 0.232 mmol, 1 equiv) in pyridine (1.5 mL) was stirred for 16 h at room temperature under an air atmosphere. The resulting mixture was concentrated under reduced pressure. The resulting mixture was diluted with water (10 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. This resulted in N-(6-methoxy-1-{[2-(trimethylsilyl)ethoxy]methyl}indazol-7-yl)-6-[4-(trifluoromethyl)pyrazol-1-yl]pyridine-3-sulfonamide (175-3, 150 mg, 76.27%) as a brown crude solid. The crude product was used in the next step directly without further purification.

Step 4: Synthesis of N-(6-methoxy-1H-indazol-7-yl)-6-(4-(trifluoromethyl)-1H-pyrazol-1-yl)pyridine-3-sulfonamide (I-175): A solution of N-(6-methoxy-1-{[2-(trimethylsilyl)ethoxy]methyl}indazol-7-yl)-6-[4-(trifluoromethyl)pyrazol-1-yl]pyridine-3-sulfonamide (114 mg, 0.200 mmol, 1 equiv) and trifluoroacetaldehyde (1 mL) in DCM (1 mL) was stirred for 2 h at room temperature under an air atmosphere. The resulting mixture was diluted with water (20 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in N-(6-methoxy-1H-indazol-7-yl)-6-[4-(trifluoromethyl)pyrazol-1-yl]pyridine-3-sulfonamide (I-175, 18.3 mg, 20.51%) as a white solid. LCMS (ES, m/z): [M+H]⁺=439; 1H NMR (400 MHz, DMSO-d₆) δ 3.24 (s, 3H), 6.79 (d, J=8.8 Hz, 1H), 7.64 (d, J=8.8 Hz, 1H), 7.99 (d, J=1.6 Hz, 1H), 8.14 (d, J=8.8 Hz, 1H), 8.24 (dd, J=8.8, 2.4 Hz, 1H), 8.39 (s, 1H), 8.64 (d, J=2.4 Hz, 1H), 9.31 (s, 1H), 10.06 (s, 1H), 13.02 (s, 1H).

### Example 303—Synthesis of 6-(2-Isopropylthiazol-5-yl)-N-(1-Methyl-1H-Indazol-7-yl)Pyridine-3-Sulfonamide (I-266)

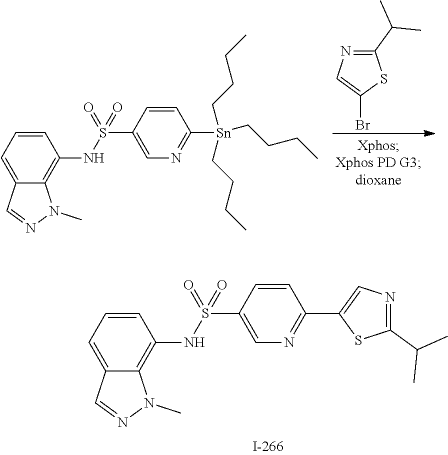

A solution of N-(1-methylindazol-7-yl)-6-(tributylstannyl)pyridine-3-sulfonamide (100 mg, 0.173 mmol, 1 equiv), 5-bromo-2-isopropyl-1,3-thiazole (42.83 mg, 0.208 mmol, 1.2 equiv), XPhos (16.51 mg, 0.035 mmol, 0.2 equiv), and XPhos Pd G₃ (14.66 mg, 0.017 mmol, 0.1 equiv) in dioxane (4 mL) was stirred for 2 hours at 90° C. under a nitrogen atmosphere. The mixture was allowed to cool to room temperature. The resulting mixture was diluted with water (20 mL). The resulting mixture was extracted with EtOAc (3×10 mL). The combined organic layers were washed with brine (1×25 mL) and dried over anhydrous Na₂SO₄. After filtration, the filtrate was concentrated under reduced pressure. The crude product was purified by Prep-HPLC with the following conditions (Column: XBridge Shield RP18 OBD Column, 30*150 mm, 5 m; Mobile Phase A: Water(10 mmol/L NH₄HCO₃⁺0.05% NH₃H₂O), Mobile Phase B: ACN; Flow rate: 60 mL/min; Gradient: 15% B to 33% B in 10 min; Wave Length: 254 nm/220 nm; RT1(min): 9.47; Number of Runs: 5) to afford 6-(2-isopropyl-1,3-thiazol-5-yl)-N-(1-methylindazol-7-yl)pyridine-3-sulfonamide (I-266, 2.1 mg, 2.90%) as an off-white solid. LC-MS (ES, m/z): [M+H]⁺414. ¹H-NMR: (400 MHz, Chloroform-d) δ 1.46 (d, J=6.9 Hz, 6H), 3.33-3.40 (m, 1H), 4.41 (s, 3H), 6.37 (s, 1H), 6.56 (d, J=7.2 Hz, 1H), 6.94 (t, J=7.6 Hz, 1H), 7.70 (d, J=7.2 Hz, 2H), 7.90 (dd, J=8.4, 2.4 Hz, 1H), 8.02 (s, 1H), 8.24 (s, 1H), 8.85 (dd, J=2.4, 0.8 Hz, 1H).

### Example 304—Synthesis of 6-(2-(2-Methoxypropan-2-yl)Thiazol-5-yl)-N-(1-Methyl-1H-Indazol-7-yl)Pyridine-3-Sulfonamide (I-268)

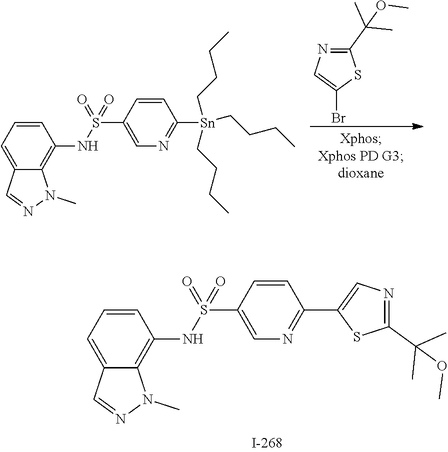 [SMILES: CCC[CH2][Sn]([CH2]CCC)([CH2]CCC)[c]1ccc(S(=O)(=O)Nc2cccc3cnn(C)c23)cn1 ; COC(C)(C)c1ncc(Br)s1 ; COC(C)(C)c1ncc(-c2ccc(S(=O)(=O)Nc3cccc4cnn(C)c34)cn2)s1]

A solution of N-(1-methylindazol-7-yl)-6-(tributylstannyl)pyridine-3-sulfonamide (100 mg, 0.173 mmol, 1 equiv), 5-bromo-2-(2-methoxypropan-2-yl)-1,3-thiazole (49.08 mg, 0.208 mmol, 1.2 equiv), XPhos (16.51 mg, 0.035 mmol, 0.2 equiv), and XPhos Pd G₃ (14.66 mg, 0.017 mmol, 0.1 equiv) in dioxane (4 mL) was stirred for 2 hours at 90° C. under a nitrogen atmosphere. The mixture was allowed to cool to room temperature. The resulting mixture was diluted with water (20 mL). The resulting mixture was extracted with EtOAc (3×10 mL). The combined organic layers were washed with brine (1×25 mL) and dried over anhydrous Na₂SO₄. After filtration, the filtrate was concentrated under reduced pressure. The crude product was purified by Prep-HPLC with the following conditions (Column: XBridge Prep OBD C18 Column, 30*150 mm, 5 m; Mobile Phase A: Water(10 mmol/L NH₄HCO₃⁺0.05% NH₃H₂O), Mobile Phase B: ACN; Flow rate: 60 mL/min; Gradient: 15% B to 38% B in 7 min; Wave Length: 254 nm/220 nm; RT1(min): 6.28; Number of Runs: 2) to afford 6-(2-(2-methoxypropan-2-yl)thiazol-5-yl)-N-(1-methyl-1H-indazol-7-yl)pyridine-3-sulfonamide (I-268, 11.8 mg, 14.95%) as an off-white solid. LC-MS (ES, m/z): [M+H]⁺=444. ¹H-NMR: (400 MHz, DMSO-d6): δ 1.56 (s, 6H), 3.21 (s, 3H), 4.31 (s, 3H), 6.60 (d, J=7.2 Hz, 1H), 6.84 (t, J=7.6 Hz, 1H), 7.40 (s, 1H), 7.96 (s, 1H), 8.07 (dd, J=8.4, 2.4 Hz, 1H), 8.11 (d, J=8.8 Hz, 1H), 8.49 (s, 1H), 8.74 (dd, J=2.4, 1.2 Hz, 1H), 10.30 (s, 1H).

### Example 305—Synthesis of 6-(2-Methyl-1-(Trifluoromethyl)-1H-Imidazol-5-yl)-N-(1-Methyl-1H-Indazol-7-yl)Pyridine-3-Sulfonamide (I-271)

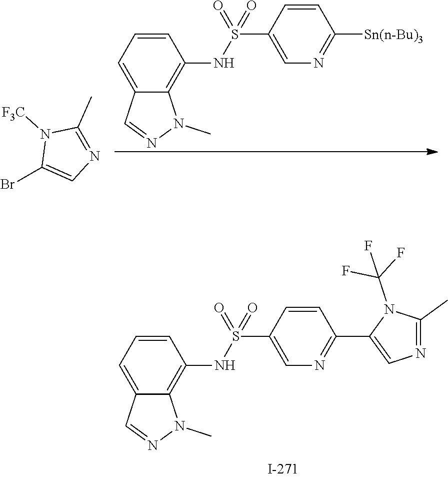

To a stirred solution of 4-bromo-2-methyl-1-(trifluoromethyl)imidazole (237.97 mg, 1.038 mmol, 3 equiv) and N-(1-methylindazol-7-yl)-6-(tributylstannyl)pyridine-3-sulfonamide (200 mg, 0.346 mmol, 1.00 equiv) in 1,4-dioxane (2 mL) were added Xphos (33.03 mg, 0.069 mmol, 0.2 equiv) and Xphos Pd G3 (58.64 mg, 0.069 mmol, 0.2 equiv) at room temperature under a nitrogen atmosphere. The resulting mixture was stirred overnight at 110° C. under a nitrogen atmosphere. The resulting mixture was diluted with water (10 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water, 0% to 100% gradient in 30 min; detector, UV 254 nm. The crude product (120 mg) was purified by Prep-HPLC with the following conditions (Column: XBridge Prep OBD C18 Column, 30*150 mm, 5 m; Mobile Phase A: Water (10 mmol/L NH₄HCO₃)+0.05% NH₃·H₂O, Mobile Phase B: ACN; Flow rate: 60 mL/min; Gradient: 10% B to 37% B in 7 min; Wave Length: 254 nm/220 nm; RT1(min): 7.83) to afford 6-(2-methyl-1-(trifluoromethyl)-1H-imidazol-5-yl)-N-(1-methyl-1H-indazol-7-yl)pyridine-3-sulfonamide (I-271, 10.2 mg, 6.64%) as an off-white solid. LC-MS: (ES, m/z): [M+1]=437. ¹H-NMR: (400 MHz, DMSO-d₆) δ 2.65 (s, 3H), 4.30 (s, 3H), 6.58 (d, J=7.2 Hz, 1H), 6.85 (t, J=7.6 Hz, 1H), 7.44 (s, 1H), 7.46 (s, 1H), 7.83 (d, J=8.4 Hz, 1H), 7.98 (d, J=1.6 Hz, 1H), 8.12 (dd, J=8.0, 2.4 Hz, 1H), 8.80 (d, J=2.4 Hz, 1H), 10.34 (s, 1H).

### Example 306—Synthesis of N-(1-Methyl-1H-Indazol-7-yl)-6-(4,5,6,7-Tetrahydropyrazolo[1,5-A]Pyridin-3-yl)Pyridine-3-Sulfonamide (I-272)

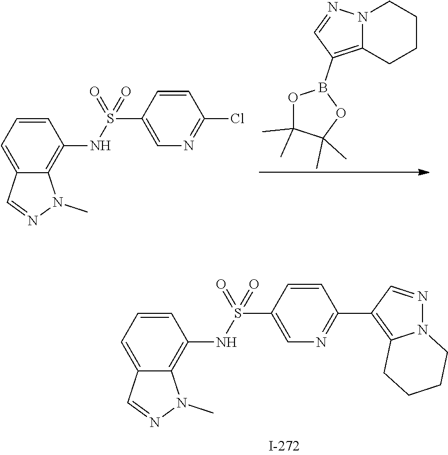

A solution of 6-chloro-N-(1-methylindazol-7-yl)pyridine-3-sulfonamide (200 mg, 0.620 mmol, 1 equiv) in dioxane (4 mL) and H₂O (1 mL) was treated with 3-(4,4,5,5-tetramethyl-1,3,2-dioxaborolan-2-yl)-4H,5H,6H-pyrrolo[1,2-b]pyrazole (174.1 mg, 0.744 mmol, 1.2 equiv) for 2 minutes at room temperature under a nitrogen atmosphere after which Pd(dppf)Cl₂ (90.68 mg, 0.124 mmol, 0.2 equiv) and Cs₂CO₃ (141.5 mg, 1.860 mmol, 3 equiv) were added in portions at 90° C. The resulting mixture was stirred for an additional 2 hours at 90° C. The resulting mixture was concentrated under reduced pressure. The residue was purified by reversed-phase flash chromatography with the following conditions: column, C18 silica gel; mobile phase, MeCN in water (0.1% FA), 0% to 50% gradient in 20 min; detector, UV 254 nm. This resulted in N-(1-methyl-1H-indazol-7-yl)-6-(4,5,6,7-tetrahydropyrazolo[1,5-a]pyridin-3-yl)pyridine-3-sulfonamide (1-272, 53.9 mg, 34.50%) as a white solid. LC-MS: (ES, m/z): [M+H]⁺=409. ¹H-NMR: (400 MHz, DMSO, ppm): 61.85 (s, 2H), 1.99 (t, J=6.0 Hz, 2H), 3.12 (t, J=6.4 Hz, 2H), 4.13 (t, J=6.0 Hz, 2H), 4.27 (s, 3H), 6.51 (d, J=7.2 Hz, 1H), 6.93 (t, J=7.6 Hz, 1H), 7.68 (d, J=8.0 Hz, 1H), 7.81 (d, J=8.4 Hz, 1H), 7.94 (dd, J=8.4, 2.5 Hz, 1H), 8.10 (d, J=10.4 Hz, 2H), 8.66 (d, J=2.4 Hz, 1H), 10.16 (s, 1H).

### Example 307—Synthesis of 6-(4-(Tert-Butyl)-1H-Pyrazol-1-yl)-N-(1-Methyl-1H-Indazol-7-yl)Pyridine-3-Sulfonamide (I-276)

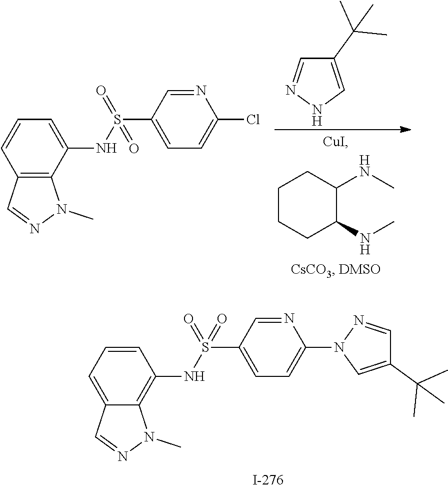

A solution/mixture of 6-chloro-N-(1-methylindazol-7-yl)pyridine-3-sulfonamide (100 mg, 0.310 mmol, 1 equiv), 4-tert-butyl-1H-pyrazole (115.43 mg, 0.930 mmol, 3 equiv), Cs₂CO₃ (302.83 mg, 0.930 mmol, 3 equiv), CuI (59.00 mg, 0.310 mmol, 1 equiv), and (1R,2R)—N1,N2-dimethylcyclohexane-1,2-diamine (22.03 mg, 0.155 mmol, 0.5 equiv) in DMSO (10 mL) was stirred overnight at 120° C. under a nitrogen atmosphere. The reaction was quenched with water at room temperature. The resulting mixture was extracted with EtOAc (3×50 mL). The combined organic layers were washed with brine (2×10 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 50% to 60% gradient in 10 min; detector, UV 254 nm. The product, 6-(4-tert-butylpyrazol-1-yl)-N-(1-methylindazol-7-yl)pyridine-3-sulfonamide (12.0 mg, 9.33%), was obtained as an off-white solid. LC-MS: (ES, m/z): [M+H]⁺=411. ¹H-NMR: (400 MHz, DMSO, ppm): δ 1.29 (s, 9H), 4.29 (s, 3H), 6.50 (d, J=7.2 Hz, 1H), 6.94 (t, J=7.6 Hz, 1H), 7.70 (d, J=8.0 Hz, 1H), 7.93 (s, 1H), 8.04-8.12 (m, 2H), 8.19 (dd, J=8.8, 2.4 Hz, 1H), 8.45 (s, 1H), 8.55 (d, J=2.4 Hz, 1H), 10.27 (s, 1H).

### Example 308—Synthesis of 6-(6,7-Dihydro-5H-Pyrazolo[5,1-B][1,3]Oxazin-3-yl)-N-(1-Methyl-1H-Indazol-7-yl)Pyridine-3-Sulfonamide (I-302)

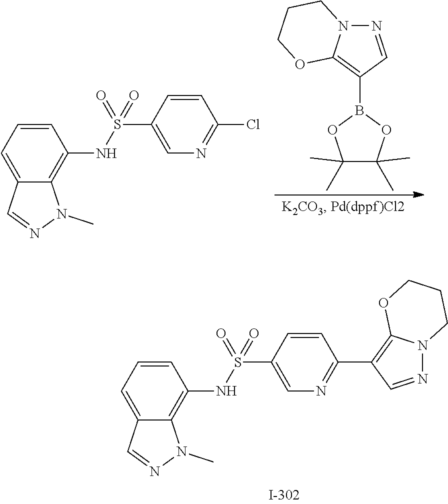 [SMILES: Cn1ncc2cccc(NS(=O)(=O)c3ccc(Cl)nc3)c21 ; CC1(C)OB(c2cnn3c2OCCC3)OC1(C)C ; Cn1ncc2cccc(NS(=O)(=O)c3ccc(-c4cnn5c4OCCC5)nc3)c21]

To a stirred solution of 6-chloro-N-(1-methylindazol-7-yl)pyridine-3-sulfonamide (100 mg, 0.310 mmol, 1 equiv) and 3-(4,4,5,5-tetramethyl-1,3,2-dioxaborolan-2-yl)-5H,6H,7H-pyrazolo[3,2-b][1,3]oxazine (116.23 mg, 0.465 mmol, 1.5 equiv) in dioxane (4 mL) and H₂O (1 mL) were added Pd(dppf)Cl₂ (22.67 mg, 0.031 mmol, 0.1 equiv) and K₂CO₃ (128.46 mg, 0.930 mmol, 3 equiv). The resulting mixture was stirred for 4 hours at 100° C. under a nitrogen atmosphere. The reaction was quenched with water at room temperature. The resulting mixture was extracted with EtOAc (2×50 mL). The combined organic layers were washed with brine (1×50 mL) and dried over anhydrous Na₂SO₄. After filtration, the filtrate was concentrated under reduced pressure. The crude product (40 mg) was purified by Prep-HPLC with the following conditions (Column: Xselect CSH C18 OBD Column 30*150 mm 5 m; Mobile Phase A: Water (0.1% FA), Mobile Phase B: ACN; Flow rate: 60 mL/min; Gradient: 25% B to 35% B in 7 min; Wave Length: 254 nm/220 nm; RT1(min): 6.7) to afford N-(1-methylindazol-7-yl)-6-{5H,6H,7H-pyrazolo[3,2-b][1,3]oxazin-3-yl}pyridine-3-sulfonamide (1-302, 12.7 mg, 9.90%) as an off-white solid. LC-MS: (ES, m/z): [M+H]⁺=411. ¹H-NMR: (400 MHz, DMSO, ppm) δ 2.25 (m, J=5.8 Hz, 2H), 4.15 (t, J=6.2 Hz, 2H), 4.29 (s, 3H), 4.46-4.53 (m, 2H), 6.55 (d, J=7.2 Hz, 1H), 6.86 (t, J=7.8 Hz, 1H), 7.49 (s, 1H), 7.64 (d, J=8.6 Hz, 1H), 7.97-7.88 (m, 2H), 8.00 (s, 1H), 8.63 (d, J=2.4 Hz, 1H).

### Example 309—Synthesis of 6-(1-Methyl-1,4,6,7-Tetrahydropyrano[4,3-C]Pyrazol-3-yl)-N-(1-Methyl-1H-Indazol-7-yl)Pyridine-3-Sulfonamide (I-306)

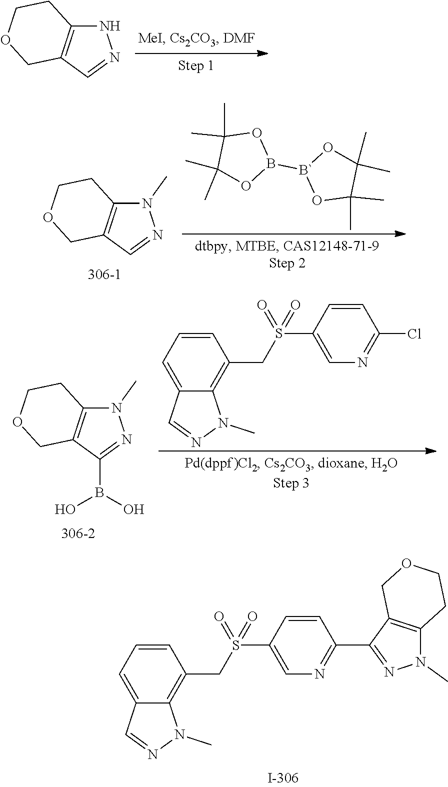

Step 1: Synthesis of 306-1: To a stirred solution of 1H,4H,6H,7H-pyrano[4,3-c] pyrazole (1.9 g, 15.305 mmol, 1 equiv) in DMF (20 mL) were added Cs₂CO₃ (9.97 g, 30.610 mmol, 2 equiv) and Mel (4.34 g, 30.610 mmol, 2 equiv) dropwise at 0° C. under a nitrogen atmosphere. The resulting mixture was stirred for 2 hours at room temperature under a nitrogen atmosphere. The resulting mixture was diluted with water (200 mL). The aqueous layer was extracted with EtOAc (2×100 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water, 10% to 30% gradient in 10 min; detector, UV 254 nm. The resulting mixture was concentrated under reduced pressure. The product, 1-methyl-4H,6H,7H-pyrano[4,3-c] pyrazole (1.3 g, 61.47%), was obtained as a yellow oil.

Step 2: Synthesis of 306-2: To a stirred solution of 1-methyl-4H,6H,7H-pyrano[4,3-c]pyrazole (300 mg, 2.171 mmol, 1 equiv) and 4,4,4′,4′,5,5,5′,5′-octamethyl-2,2′-bi(1,3,2-dioxaborolane) (1102.73 mg, 4.342 mmol, 2 equiv) in MTBE (5 mL) were added 4-tert-butyl-2-(4-tert-butylpyridin-2-yl)pyridine (58.28 mg, 0.217 mmol, 0.1 equiv) and (1,5-cyclooctadiene)(methoxy)iridium(I) dimer (71.96 mg, 0.109 mmol, 0.05 equiv) in portions at room temperature under nitrogen atmosphere. The resulting mixture was stirred for 2 h at 55° C. under nitrogen atmosphere. The resulting mixture was diluted with PE (50 mL) and a precipitate formed. The solids were removed by a filtration. The filtrate was concentrated under reduced pressure and diluted with hexane (100 mL). Another filtration was performed. The solids were washed with hexane (100 mL). The combined filtrate was concentrated under reduced pressure. This resulted in crude 1-methyl-3-(4,4,5,5-tetramethyl-1,3,2-dioxaborolan-2-yl)-4H,6H,7H-pyrano[4,3-c]pyrazole (500 mg, 87.19%) as a brown oil. The crude product was used in the next step directly without further purification.

Step 3: Synthesis of 6-(1-methyl-1,4,6,7-tetrahydropyrano[4,3-c]pyrazol-3-yl)-N-(1-methyl-1H-indazol-7-yl)pyridine-3-sulfonamide (1-306). To a stirred solution of 6-chloro-N-(1-methylindazol-7-yl) pyridine-3-sulfonamide (100 mg, 0.310 mmol, 1 equiv) and 1-methyl-4H,6H,7H-pyrano[4,3-c] pyrazol-3-ylboronic acid (169.15 mg, 0.930 mmol, 3 equiv) in dioxane (5 mL) and H₂O (1 mL) were added Pd(dppf)Cl₂, CH₂Cl₂ (25.24 mg, 0.031 mmol, 0.1 equiv), and K₂CO₃ (85.64 mg, 0.620 mmol, 2 equiv) in portions at room temperature under a nitrogen atmosphere. The resulting mixture was stirred for 2 hours at 100° C. under a nitrogen atmosphere. The resulting mixture was diluted with water (20 mL). The resulting mixture was extracted with EtOAc (3×20 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 40% to 50% gradient in 10 min; detector, UV 254 nm. The resulting mixture was concentrated under reduced pressure. The product, 6-{1-methyl-4H,6H,7H-pyrano[4,3-c] pyrazol-3-yl}-N-(1-methylindazol-7-yl) pyridine-3-sulfonamide (51.7 mg, 37.90%), was obtained as an off-white solid. LC-MS: (ES, m/z): [M+H]⁺=425. ¹H-NMR: (400 MHz, DMSO, ppm): δ 2.77 (t, J=4.0 Hz, 2H), 3.82 (s, 3H), 3.87 (t, J=4.0 Hz, 2H), 4.26 (s, 3H), 4.85 (s, 2H), 6.48 (dd, J=8.0, 1.0 Hz, 1H), 6.93 (t, J=8.0 Hz, 1H), 7.70 (d, J=8.0 Hz, 1H), 8.02 (dd, J=8.0, 2.4 Hz, 1H), 8.12-8.06 (m, 2H), 8.73-8.69 (m, 1H), 10.23 (s, 1H).

### Example 310—MALT1 Inhibition Assay

Inhibition of MALT1 activity by the presence of small molecules was evaluated using MALT-1 Fluorogenic Peptide Cleavage Assay. The assay utilizes a quenched AMC-labelled peptide that contains the MALT-1 recognition sequence and cleavage site (LRSR). MALT-1 mediated cleavage of the peptide relieves the quenching and leads to an increase in fluorescence at excitation (342 nm) and emission (441 nm).

The following reagents were obtained commercially and used to prepare standard reagent formulations as further described below: N-2-hydroxyethylpiperazine-N-2-ethane sulfonic acid (HEPES) (1 M, pH 7.5, stored at 4° C.), sodium citrate (stored at RT), tris(2-carboxyethyl)phosphine hydrochloride (T-CEP) (500 mM, stored at −20° C.), ethylenediaminetetraacetic acid (EDTA) (500 mM, stored at RT), dimethylsulfoxide (DMSO, stored at RT), 3-[(3-cholamidopropyl)dimethylammonio]-1-propane sulphate (CHAPS) (stored at RT), dimethylsulfoxide (DMSO), DMSO (Fisher Scientific, stored at RT), Avi-tagged FL MALT-1 (Pharmaron, stored at −70° C.), Ac-LRSR-AMC Peptide (SM Biochemicals, stored at −20° C.)., and ultrapure water (MILLI-Q®).

The standard reagent formulations used in this assay were prepared and stored as follows. A 1.1 M solution of sodium citrate (161.3 g, 500 mL) was prepared and stored at room temperature. A 10% (w/v) CHAPS solution (2.0 g, 20 mL) was prepared and stored at 4° C. A 500 mM HEPES solution having pH of 6.89 was prepared from 200 mL of a 1 M HEPES solution having a pH of 7.5 using concentrated hydrochloric acid and brought to a final volume of 400 mL with MILLI-Q® H₂O. The substrate, 10 mM Ac-LRSR-AMC Peptide, was prepared (10 mg, 1.370 mL DMSO) and stored at −20° C.

Compounds were plated to provide a 2% DMSO final concentration using a ProxiPlate-384 Plus F Black 384-shallow well microplate. The assay-ready plates were equilibrated to room temperature. A reaction buffer (30 mL total volume) was prepared by combining HEPES (pH 6.89, 25 mM, 1.5 mL), sodium citrate (660 mM, 18.0 mL), T-CEP (1 mM, 0.06 mL), EDTA (0.1 mM, 0.06 mL), CHAPS (0.05%, 0.15 mL), DMSO (2%, 0.6 mL), and MILLI-Q® H₂O (10.23 mL; 9.63 mL when backfilled to 2% DMSO) followed by thorough mixing. DMSO was added only when compound plates had not been DMSO-backfilled to 2%.

MALT-1 was thawed and kept on ice. Peptide substrate was thawed on the bench under ambient conditions. A MALT-1 enzyme working stock was prepared from Avi-tagged FL MALT-1 (40 nM in a prepared reagent volume of 16.5 μL) and the reaction buffer (13.0 mL). MALT1 working stock (5 μL) was added to each well of the microplate. MALT-1 was pre-incubated with compounds for 30 minutes at room temperature.

Two substrate working stocks (Km and 10×Km) were prepared. The 1×Km substrate was prepared from Ac-LRSR-AMC Peptide (50 μM in a prepared reagent volume of 35.0 μL) and the reaction buffer (6.965 mL). The 10× Km substrate was prepared from Ac-LRSR-AMC Peptide (280 μM in a prepared reagent volume of 196.0 μL) and the reaction buffer (6.804 mL). The reaction was initiated by the addition of substrate working stock (5 μL, 50 μM) to Km plates and the addition of substrate working stock (5 μL 280 μM) to 10×Km plates. The plates were covered and incubated on the bench at room temperature for 90 minutes.

Fluorescence intensity was determined using a CLARIOstar microplate reader (BMG LABTECH) using the optimised AMC mode. Before taking readings, a 70% gain was applied on the neutral control well. Compound data were normalised to % Inhibition and used to plot sigmoidal concentration response curves to yield various parameters including IC₅₀.

The results of the MALT1 assay are reported in Table 4, below. Compounds with an IC₅₀ less than or equal to 400 nM are designated as “A”. Compounds with an IC₅₀ greater than 400 nM and less than or equal to 1000 nM are designated as “B”. Compounds with an IC₅₀ greater than 1000 nM and less than or equal to 2500 nM are designated as “C”. Compounds with an IC₅₀ greater than 2500 nM are designated as “D”. Compounds in which an IC₅₀ was not measured are designated as “N/A”.

### Example 311—NF-KB Reporter Assay

Jurkat cells were maintained in complete RPMI 1640 media supplemented with 10% FBS. Prior to the assay, 120 nL of the serial diluted compound was added in each well of 384-well flat bottom white plate (Corning #3570) by using liquid handler Echo550. Jurkat cells were harvested by centrifugation at 120 g for 10 min and resuspended in fresh RPMI 1640 media without serum. 30 μL of cells (25,000 cells) were seeded in each well of 384-well plate containing compound. After 1 hr incubation at 37° C. in a 5% CO₂ incubator, 10 μL of diluted aCD3/aCD28/PMA (final concentration at 5 μg/mL, 5 μg/mL and 0.03 μg/mL, respectively) in RPMI 1640 without serum were added to each well. After incubation at 37° C. in 5% CO₂ for 4 hr, 30 μL of One-Glo reagent (Promega #E6120) was added into each well. The plate was left at room temperature for 5 min and the signal was measured on EnVision (PerkinElmer).

The results of the NF-kB reporter assay are reported in Table 4, below. Compounds with an IC₅₀ less than or equal to 400 nM are designated as “A”. Compounds with an IC₅₀ greater than 400 nM and less than or equal to 1000 nM are designated as “B”. Compounds with an IC₅₀ greater than 1000 nM and less than or equal to 2500 nM are designated as “C”. Compounds with an IC₅₀ greater than 2500 nM are designated as “D”. Compounds in which an IC₅₀ was not measured are designated as “N/A”.

TABLE 4
MALT1 inhibition and NF-kB reporter assay data

| | MALT1 | NF-kB |
|---|---|---|
| | Inhibition | Reporter |
| Example # | IC₅₀ | Assay IC₅₀ |
| I-1 | A | A |
| I-2 | A | A |
| I-3 | A | N/A |
| I-4 | B | N/A |
| I-5 | A | N/A |
| I-6 | A | A |
| I-7 | C | C |
| I-8 | D | D |
| I-9 | D | D |
| I-10 | A | A |
| I-11 | A | A |
| I-12 | A | A |
| I-13 | A | A |
| I-14 | A | A |
| I-15 | C | A |
| I-16 | A | A |
| I-17 | A | A |
| I-18 | A | A |
| I-19 | D | A |
| I-20 | C | B |
| I-21 | D | A |
| I-22 | A | A |
| I-23 | B | A |
| I-24 | C | A |
| I-25 | C | D |
| I-26 | A | D |
| I-27 | A | C |
| I-28 | B | A |
| I-29 | A | A |
| I-30 | A | A |
| I-31 | A | A |
| I-32 | A | A |
| I-33 | A | A |
| I-34 | A | B |
| I-35 | D | A |
| I-36 | D | A |
| I-37 | D | C |
| I-38 | C | A |
| I-39 | D | B |
| I-40 | D | A |
| I-41 | A | B |
| I-42 | D | A |
| I-43 | C | A |
| I-44 | B | A |
| I-45 | A | N/A |
| I-46 | A | N/A |
| I-47 | C | N/A |
| I-48 | A | A |
| I-49 | A | A |
| I-50 | B | A |
| I-51 | C | A |
| I-52 | D | C |
| I-53 | D | B |
| I-55 | D | A |
| I-56 | D | A |
| I-57 | D | A |
| I-58 | C | A |
| I-59 | D | A |
| I-62 | D | A |
| I-63 | A | A |
| I-64 | D | D |
| I-65 | C | A |
| I-66 | B | A |
| I-67 | D | A |
| I-68 | A | A |
| I-69 | A | A |
| I-70 | A | A |
| I-71 | C | A |
| I-72 | C | A |
| I-73 | B | B |
| I-74 | A | A |
| I-75 | A | A |
| I-76 | D | A |
| I-77 | D | D |
| I-79 | D | C |
| I-80 | D | C |
| I-81 | D | B |
| I-82 | D | B |
| I-83 | D | A |
| I-84 | B | A |
| I-85 | D | A |
| I-86 | D | A |
| I-87 | D | A |
| I-88 | D | A |
| I-89 | D | A |
| I-90 | D | B |
| I-98 | D | A |
| I-101 | A | A |
| I-103 | B | A |
| I-104 | A | A |
| I-105 | D | D |
| I-106 | A | A |
| I-107 | D | A |
| I-108 | D | A |
| I-109 | D | A |
| I-110 | D | A |
| I-111 | D | A |
| I-112 | D | D |
| I-113 | D | B |
| I-114 | D | A |
| I-115 | D | D |
| I-116 | D | B |
| I-117 | D | D |
| I-118 | D | D |
| I-119 | D | A |
| I-120 | D | A |
| I-121 | D | A |
| I-122 | D | A |
| I-123 | D | B |
| I-124 | D | D |
| I-125 | D | A |
| I-126 | A | A |
| I-127 | A | A |
| I-128 | C | B |
| I-129 | A | A |
| I-130 | D | C |
| I-131 | D | A |
| I-132 | D | A |
| I-133 | D | D |
| I-134 | D | A |
| I-135 | D | B |
| I-136 | D | D |
| I-137 | D | D |
| I-138 | D | A |
| I-139 | D | A |
| I-140 | A | A |
| I-141 | A | A |
| I-142 | A | A |
| I-143 | A | A |
| I-144 | D | D |
| I-145 | D | A |
| I-146 | D | D |
| I-147 | D | B |
| I-148 | A | A |
| I-149 | A | A |
| I-150 | A | A |
| I-151 | A | A |
| I-152 | D | A |
| I-153 | A | A |
| I-154 | D | D |
| I-155 | D | A |
| I-156 | A | A |
| I-157 | A | A |
| I-158 | D | D |
| I-159 | A | A |
| I-160 | C | A |
| I-161 | C | B |
| I-162 | A | A |
| I-163 | A | C |
| I-164 | A | B |
| I-165 | A | A |
| I-166 | D | D |
| I-167 | C | D |
| I-168 | D | D |
| I-169 | C | D |
| I-170 | D | D |
| I-171 | D | A |
| I-172 | A | B |
| I-173 | A | A |
| I-174 | A | A |
| I-175 | A | A |
| I-176 | D | D |
| I-177 | D | D |
| I-178 | D | D |
| I-179 | D | D |
| I-180 | B | C |
| I-181 | D | B |
| I-182 | D | D |
| I-183 | A | A |
| I-184 | A | A |
| I-185 | A | A |
| I-186 | A | A |
| I-187 | A | A |
| I-188 | A | A |
| I-189 | C | C |
| I-190 | D | D |
| I-191 | A | A |
| I-192 | A | A |
| I-193 | A | A |
| I-194 | A | A |
| I-195 | A | A |
| I-196 | A | A |
| I-197 | A | A |
| I-198 | D | C |
| I-199 | D | C |
| I-200 | A | A |
| I-201 | D | D |
| I-202 | B | A |
| I-203 | D | A |
| I-204 | A | C |
| I-205 | C | C |
| I-206 | A | A |
| I-207 | C | A |
| I-208 | C | A |
| I-209 | D | A |
| I-210 | D | A |
| I-211 | D | B |
| I-212 | A | A |
| I-213 | A | A |
| I-214 | B | A |
| I-215 | D | D |
| I-216 | D | D |
| I-217 | D | D |
| I-218 | D | D |
| I-219 | D | A |
| I-220 | D | B |
| I-221 | D | C |
| I-222 | D | A |
| I-223 | D | A |
| I-224 | D | A |
| I-225 | B | A |
| I-226 | D | A |
| I-227 | C | A |
| I-228 | C | A |
| I-229 | A | A |
| I-230 | D | D |
| I-231 | D | D |
| I-232 | D | D |
| I-233 | B | A |
| I-234 | D | A |
| I-235 | A | B |
| I-236 | B | D |
| I-237 | A | A |
| I-238 | A | A |
| I-239 | C | D |
| I-240 | A | A |
| I-241 | B | A |
| I-242 | B | A |
| I-243 | D | A |
| I-244 | D | A |
| I-245 | D | A |
| I-246 | D | A |
| I-247 | D | A |
| I-248 | D | D |
| I-249 | A | B |
| I-250 | C | A |
| I-251 | D | A |
| I-252 | D | A |
| I-253 | D | B |
| I-264 | D | A |
| I-265 | D | A |
| I-266 | D | A |
| I-267 | D | B |
| I-268 | D | A |
| I-269 | D | A |
| I-270 | D | A |
| I-271 | D | A |
| I-272 | D | A |
| I-273 | A | B |
| I-274 | D | A |
| I-275 | D | B |
| I-276 | D | A |
| I-277 | D | B |
| I-278 | D | D |
| I-279 | D | A |
| I-280 | B | A |
| I-281 | C | A |
| I-282 | D | B |
| I-283 | D | A |
| I-284 | B | A |
| I-285 | B | A |
| I-286 | A | A |
| I-287 | C | A |
| I-288 | D | A |
| I-289 | D | B |
| I-290 | A | A |
| I-291 | A | A |
| I-292 | D | B |
| I-293 | D | B |
| I-294 | D | C |
| I-295 | D | A |
| I-296 | C | A |
| I-297 | D | D |
| I-298 | D | A |
| I-299 | D | D |
| I-300 | A | B |
| I-301 | C | C |
| I-302 | D | A |
| I-303 | D | B |
| I-304 | A | A |
| I-305 | D | B |
| I-306 | D | A |
| I-307 | D | B |
| I-308 | C | A |
| I-309 | D | A |
| I-310 | D | D |
| I-311 | D | D |
| I-312 | C | B |
| I-313 | D | A |
| I-314 | D | C |
| I-315 | D | D |
| I-316 | A | A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 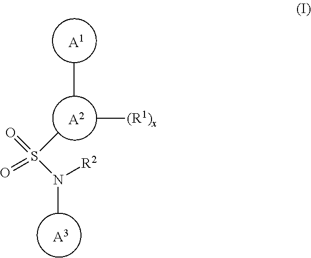
or a pharmaceutically acceptable salt thereof; wherein:
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, C₂₋₄ aminoalkyl, —(C₁₋₄ alkylene)-(C₁₋₆ alkoxyl), or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl substituted with 0 or 1 occurrences of C₁₋₆ alkoxyl, C₃₋₇ halocycloalkyl, C₃₋₇ hydroxycycloalkyl, —O—C₃₋₇ cycloalkyl, nitro, —C(O)R⁷, —N(R⁸)(R⁹), oxo, C₁₋₄ hydroxyalkyl, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, C₁₋₄ alkyl, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, 3, or 4 heteroatoms independently selected from oxygen, nitrogen, and sulfur, a 9-10 membered bicyclic heteroaryl containing 1, 2, or 3 heteroatoms independently selected from oxygen, nitrogen, and sulfur, or —N(R²)-(5-6 membered saturated carbocylic ring substituted with n occurrences of R⁶), wherein the heteroaryl and heterocyclyl are substituted with n occurrences of R⁶;
A² is a pyridinylene, pyridazinylene, pyrimidinylene, or phenylene; 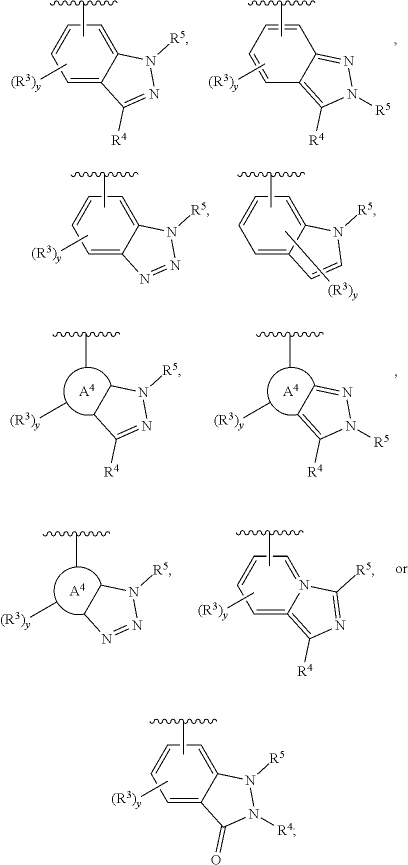
A⁴ is a 6-membered aromatic ring containing 1 nitrogen atom;
y is 0, 1, or 2;
m and x are independently 0, 1, or 2; and
n is 0, 1, 2, or 3;
provided that if A² is phenylene, then y is 1 or 2 and at least one occurrence of R³ is C₁₋₆ alkoxyl, C₁₋₆ deuteroalkoxyl, or C₃₋₇ cycloalkyl.

2. The compound of claim 1, wherein the compound is a compound of Formula I.

3. The compound of claim 1, wherein the compound is represented by Formula I-1: 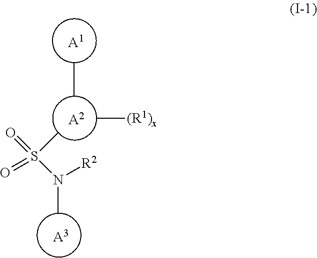
or a pharmaceutically acceptable salt thereof; wherein:
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, C₃₋₇ halocycloalkyl, C₃₋₇ hydroxycycloalkyl, —O—C₃₋₇ cycloalkyl, nitro, —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, or 3 heteroatoms independently selected from oxygen, nitrogen, and sulfur, or a 9-10 membered bicyclic heteroaryl containing 1, 2, or 3 heteroatoms independently selected from oxygen, nitrogen, and sulfur, wherein the heteroaryl and heterocyclyl are substituted with n occurrences of R⁶;
A² is a pyridinylene, pyridazinylene, pyrimidinylene, or phenylene;
A³ is 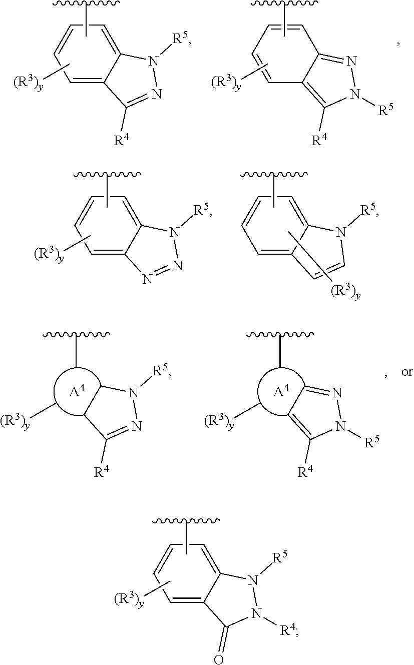
A⁴ is a 6-membered aromatic ring containing 1 nitrogen atom;
y is 0, 1, or 2; and
m, n, and x are independently 0, 1, or 2;
provided that if A² is phenylene, then y is 1 or 2 and at least one occurrence of R³ is C₁₋₆ alkoxyl, C₁₋₆ deuteroalkoxyl, or C₃₋₇ cycloalkyl.

4-8. (canceled)

9. The compound of claim 1, wherein the compound is a compound of Formula Ia or a pharmaceutically acceptable salt thereof:

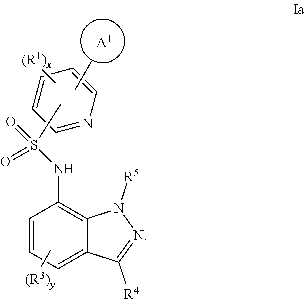

10. The compound of claim 1, wherein the compound is a compound of Formula Ib or a pharmaceutically acceptable salt thereof:

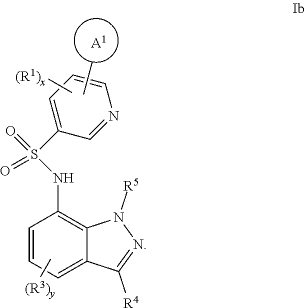

11. The compound of claim 1, wherein the compound is a compound of Formula Ic or a pharmaceutically acceptable salt thereof:

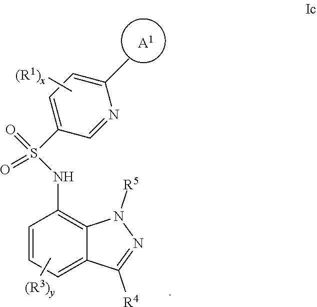

12. The compound of claim 1, wherein the compound is a compound of Formula Id or a pharmaceutically acceptable salt thereof:

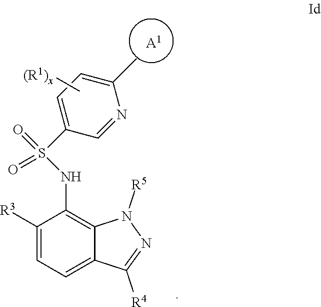

13-14. (canceled)

15. The compound of claim 10, wherein A¹ is a 5-6 membered heteroaryl containing 1, 2, or 3 heteroatoms independently selected from oxygen, nitrogen, and sulfur, or a 9-10 membered bicyclic heteroaryl containing 1, 2, or 3 heteroatoms independently selected from oxygen, nitrogen, and sulfur; wherein the heteroaryl are substituted with n occurrences of R⁶.

16. The compound of claim 10, wherein A¹ is a 5-membered heteroaryl containing 1, 2, or 3 heteroatoms independently selected from oxygen, nitrogen, and sulfur, wherein the heteroaryl is substituted with n occurrences of R⁶.

17. The compound of claim 10, wherein A¹ is pyrazolyl, imidazolyl, oxazolyl, isoxazolyl, isothiazolyl, pyrrolyl, or furanyl, each of which is substituted with n occurrences of R⁶.

18. The compound of claim 10, wherein A¹ is pyrazolyl substituted with n occurrences of R⁶.

19. The compound of claim 10, wherein A¹ is 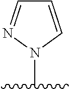 [SMILES: *n1cccn1]
substituted with n occurrences of R⁶.

20. The compound of claim 19, wherein n is 1.

21. (canceled)

22. The compound of claim 10, wherein A¹ is

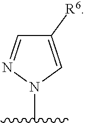

23. The compound of claim 22, wherein R⁶ is C₁₋₆ haloalkyl.

24. The compound of claim 22, wherein R₆ is —CF₃.

25. The compound of claim 22, wherein R₆ is C₃₋₇ cycloalkyl.

26. The compound of claim 22, wherein R⁶ is cyclopropyl.

27-28. (canceled)

29. The compound of claim 10, wherein R⁵ is C₁₋₄ alkyl.

30. The compound of claim 10, wherein R⁵ is methyl.

31. The compound of claim 10, wherein R⁴ is hydrogen.

32-35. (canceled)

36. The compound of claim 10, wherein R³ is C₁₋₆ alkoxyl.

37. The compound of claim 10, wherein R³ is methoxy.

38. A compound represented by Formula II: 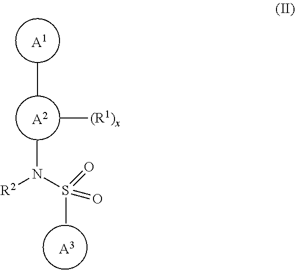
or a pharmaceutically acceptable salt thereof; wherein:
R¹ represents independently for each occurrence halo, C₁₋₄ alkyl, C₁₋₄ haloalkyl, or cyano;
R² is hydrogen, C₁₋₄ alkyl, C₂₋₄ hydroxyalkyl, or —(C₁₋₆ alkylene)-N(R⁸)(R⁹);
R⁵ is hydrogen, C₁₋₄ alkyl, or C₁₋₄ deuteroalkyl;
R³ represents independently for each occurrence halo, hydroxyl, C₁₋₆ alkyl, C₁₋₆ haloalkyl, C₁₋₆ deuteroalkyl, C₁₋₆ alkoxyl, C₁₋₆ deuteroalkoxyl, C₃₋₇ cycloalkyl, —O—C₃₋₇ cycloalkyl, cyano, —CO₂R¹⁰, —C(O)N(R⁸)(R⁹), —N(R⁸)C(O)R¹⁰, or —S(O₂)R¹⁰; or two occurrences of R³ are taken together with the intervening atoms to form a 5-7 membered ring containing 0, 1, or 2 heteroatoms independently selected from nitrogen, oxygen, and sulfur;
R⁴ is hydrogen, halo, or C₁₋₄ alkyl;
R⁶ represents independently for each occurrence halo, hydroxyl, C₁₋₆ alkyl, C₁₋₆ haloalkyl, cyano, C₁₋₆ alkoxyl, C₃₋₇ cycloalkyl, —O—C₃₋₇ cycloalkyl, C₃₋₇ halocycloalkyl, C₃₋₇ hydroxycycloalkyl, nitro, —C(O)R⁷, —C(O)N(R⁸)(R⁹), —N(R⁸)C(O)R¹⁰, —S(O₂)R¹⁰, —S(O₂)N(R⁸)(R⁹), —N(R⁸)S(O₂)R¹⁰, or a 3-7 membered saturated heterocyclyl containing 1 or 2 heteroatoms independently selected from oxygen and nitrogen, wherein the heterocyclyl is substituted with 0 or 1 halo;
R⁷ is —OH, —O—(C₁₋₆ alkyl), —O—C₃₋₇ cycloalkyl, or a 4-6 membered saturated heterocyclyl containing 1 or 2 heteroatoms independently selected from oxygen, nitrogen, and sulfur, wherein the heterocyclyl is substituted with m occurrences of R¹¹;
R⁸ and R⁹ are independently hydrogen, C₁₋₆ alkyl, or C₃₋₇ cycloalkyl, or R⁸ and R⁹ are taken together with the nitrogen atom to which they are attached to form a 3-7 membered heterocyclic ring containing 1 nitrogen atom;
R¹⁰ represents independently for each occurrence C₁₋₆ alkyl or —(C₀₋₅ alkylene)-C₃₋₇ cycloalkyl;
R¹¹ represents independently for each occurrence halo, hydroxyl, C₁₋₆ alkyl, C₁₋₆ haloalkyl, C₁₋₆ alkoxyl, or C₃₋₇ cycloalkyl;
A¹ is a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, or a 3-10 membered saturated heterocyclyl containing 1, 2, or 3 heteroatoms independently selected from oxygen, nitrogen, and sulfur, wherein the heteroaryl and heterocyclyl are substituted with n occurrences of R⁶;
A² is a pyridinylene, pyridazinylene, or pyrimidinylene;
A³ is 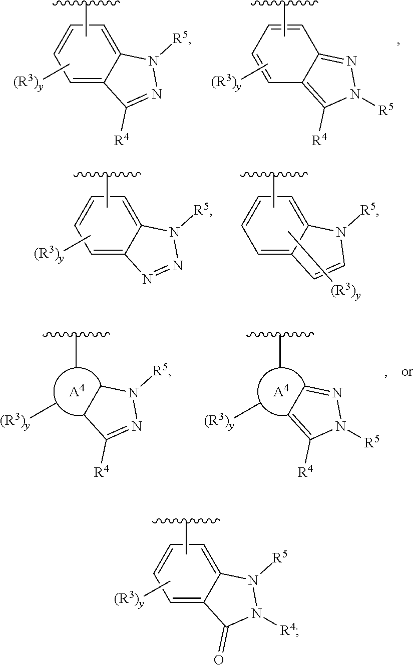
A⁴ is a 6-membered aromatic ring containing 1 nitrogen atom;
y is 1 or 2; and
m, n, and x are independently 0, 1, or 2;
provided at least one occurrence of R³ is C₁₋₆ alkoxyl, C₁₋₆ deuteroalkoxyl, or C₃₋₇ cycloalkyl.

39-47. (canceled)

48. The compound of claim 1, wherein the compound is a compound of Formula Ie: 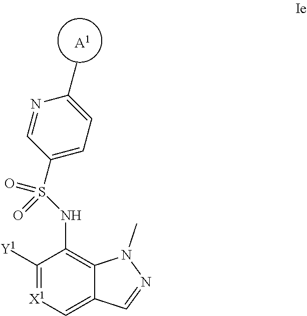
or a pharmaceutically acceptable salt thereof; wherein
X¹ is nitrogen or —C(H)—; and
Y¹ is hydrogen or —OCH₃.

49. The compound of claim 48 wherein A¹ is selected from

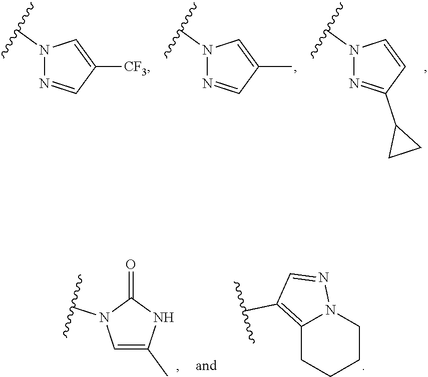

50. A compound in Table 1 or 2, or a pharmaceutically acceptable salt thereof.

51. A pharmaceutical composition comprising a compound of claim 1 and a pharmaceutically acceptable carrier.

52. A method for treating a disease or condition mediated by MALT1, comprising administering to a subject in need thereof a therapeutically effective amount of a compound of claim 1 to treat the disease or condition.

53-55. (canceled)

56. The method of claim 52, wherein said disease or condition mediated by MALT1 is selected from cancer, neoplasia, chronic inflammatory disorder, acute inflammatory disorder, auto-inflammatory disorder, autoimmune disorder, fibrotic disorder, metabolic disorder, cardiovascular disorder, cerebrovascular disorder, myeloid cell-driven hyper-inflammatory response in COVID-19 infection, and a combination thereof.

57. The method of claim 52, wherein said disease or condition mediated by MALT1 is cancer.

58. The method of claim 57, wherein the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia.

59. The method of claim 57, wherein the cancer is a lymphoma or a leukemia.

60. The method of claim 57, wherein the cancer is a B-cell lymphoma or chronic myelocytic leukemia.

61. The method of claim 52, wherein said disease or condition mediated by MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

62. The method of claim 52, wherein said disease or condition mediated by MALT1 is multiple sclerosis, ankylosing spondylitis, arthritis, osteoarthritis, juvenile arthritis, reactive arthritis, rheumatoid arthritis, psoriatic arthritis, acquired immunodeficiency syndrome (AIDS), Coeliac disease, psoriasis, chronic graft-versus-host disease, acute graft-versus-host disease, Crohn's disease, inflammatory bowel disease, multiple sclerosis, systemic lupus erythematosus, Celiac Sprue, idiopathic thrombocytopenic thrombotic purpura, myasthenia gravis, Sjogren's syndrome, scleroderma, ulcerative colitis, asthma, uveitis, rosacea, dermatitis, alopecia areata, vitiligo, arthritis, Type 1 diabetes, lupus erythematosus, systemic lupus erythematosus, Hashimoto's thyroiditis, myasthenia gravis, nephrotic syndrome, eosinophilia fasciitis, hyper IgE syndrome, lepromatous leprosy, sezary syndrome, idiopathic thrombocytopenia purpura, restenosis following angioplasty, a tumor, or artherosclerosis.

63. (canceled)

64. The method of claim 52, wherein the subject is a human.

65. A method of inhibiting the activity of MALT1, comprising contacting a MALT1 with an effective amount of a compound of claim 1 to inhibit the activity of said MALT1.
